(19) United States
(12) Patent Application Publication
(10) Pub. No.: US 2024/0229157 A1
(43) Pub. Date: Jul. 11, 2024

(54) COMPOSITIONS COMPRISING NULLOMERS AND METHODS OF USING THE SAME FOR CANCER DETECTION AND DIAGNOSIS

(71) Applicants: THE REGENTS OF THE UNIVERSITY OF CALIFORNIA, Oakland, CA (US); WELLCOME SANGER INSTITUTE, Hinxton, Cambridge (GB)

(72) Inventors: Nadav AHITUV, Walnut Creek, CA (US); Ofer YIZHAR-BARNEA, Foster City, CA (US); Ilias GEORGAKOPOUOS-SOARES, San Francisco, CA (US); Ioannis MOURATIDIS, Brussels (BE); Martin HEMBERG, Weston, MA (US)

(21) Appl. No.: 18/558,992

(22) PCT Filed: May 3, 2022
(86) PCT No.: PCT/US22/27536

§ 371 Date: Nov. 3, 2023

Related U.S. Application Data
(60) Provisional application No. 63/183,610, filed on May 3, 2021.
(60) Provisional application No. 63/230,584, filed on Aug. 6, 2021.

(51) Int. Cl. C12Q 1/6886; C12N 9/22; C12N 15/10; C12N 15/11; C12Q 1/6806; C12Q 1/6872; G16B 20/00; G16H 50/20

(52) U.S. Cl. CPC C12Q 1/6886; C12N 9/22; C12N 15/1096; C12N 15/111; C12Q 1/6806; C12Q 1/6872; G16B 20/00; G16H 50/20; C12N 2310/20; C12Q 2600/156

(57) ABSTRACT

The present disclosure provides methods and compositions for the detection, identification, classification and characterization of cancer in general and cancer types in biological material. Sequences that are not found in the human reference genome or any set of genomic tiled regions, termed nullomers, which can resurface due to mutations, serve as biomarkers and are predictive of cancer. The invention also enables the identification of cancer subtype and the stratification of patients based on sample-specific vulnerabilities guiding treatment choice. For coding nullomers it also covers their use as neoantigens. The algorithms presented hereby can be applied to biological material including biopsy, cell-free DNA samples and RNA samples.

### TECHNOLOGY FIELD

The present disclosure relates to the development of prognostic and diagnostic cancer biomarkers in biological material and the characterization of tumor subtype, vulnerabilities and therapeutic strategies, from the resurfacing of nullomers.

### BACKGROUND

Cancer is the second leading cause of death worldwide (“Cancer” n.d.), and for most cancer types, survivability is significantly higher if the tumor is detected at an early stage (Hawkes 2019; Etzioni et al. 2003). Currently mass population screening is applicable only for breast and cervical cancers and utilizes physical tests like mammography and cytology screens. Detection for other cancer types, done both en masse and in a low and affordable resource setting, still poses a major challenge for the scientific and clinical communities (“Cancer” n.d.). In particular, a major hurdle is to single-out cancer biomarkers for the detection of cancer development at its earliest stage for patient stratification and improvement of patients' outcome by providing personalized treatments.

Circulating cell-free DNA (cfDNA) is an emerging and promising resource for cancer diagnostics and prognostics (Bronkhorst, Ungerer, and Holdenrieder 2019; Heitzer, Auinger, and Speicher 2020). It has a short life span (16 minutes to 2.5 hours), which makes it a highly temporal indicator of various processes occurring in the subject's body and with advances in sequencing technologies, can be rapidly analyzed. Analysis of cell-free tumor DNA (ctDNA, liquid biopsy) has become a prospective minimally invasive tool to screen the population and to monitor patients already diagnosed with cancer. To distinguish cancerous cells, their tissue of origin and cancer type, current technologies rely on sequencing to resolve somatic mutations (Zill et al. 2018) and epigenetic marks, such as DNA methylation or histone modifications that can determine the cancerous tissue (Saghafinia et al. 2018; Sadeh et al. 2021). However, ctDNA still has many hurdles and caveats that need to be overcome (Barbany et al. 2019). Some of the major hurdles include: 1) cfDNA is fragmented (180-360 base pairs) making its collection and extraction more challenging and the tumor-derived DNA makes up only a small portion (estimated to be around 0.4%) warranting the need for extremely sensitive biomarkers that can easily detect the presence of cancerous cells; 2) prior knowledge of specific mutations or methylation marks is required for targeted screening, and consequently the main focus has been on coding mutations which only constitute a small fraction of mutations; 3) cfDNA mutation and epigenetic diagnosis could be confounded by somatic alterations in white blood cells (Razavi et al. 2019); 4) the diagnostic techniques used to detect methylation or histone marks are technologically complex and can have low sensitivity and specificity (Ji et al. 2014; Worm Ørntoft 2018; Warton and Samimi 2015; Bronkhorst, Ungerer, and Holdenrieder 2019) and 5) to provide the most optimal cancer treatment, it needs to be diagnosed at preliminary stages when the tumor is small (˜5 mm in diameter). At these stages, the tumor produces minute levels of ctDNA that are difficult to detect using current methods (Bronkhorst, Ungerer, and Holdenrieder 2019).

Nullomers are short DNA sequences (11-18 base pairs) that are absent from the human genome (Hampikian and Andersen 2006; Vergni and Santoni 2016). While the absence of nullomeric sequences could be due to chance, we and others have shown that a significant proportion of them is under negative selection pressures (Georgakopoulos-Soares et al. 2020; Vergni and Santoni 2016), suggesting that they could have a deleterious effect on the genome. Experimental evidence was also provided through the observation that two out of three nullomers led to lethality in several cancerous cell types when delivered as synthetic peptides (Alileche et al. 2012; Alileche and Hampikian 2017). It has also been shown that these sequences could be used as DNA “fingerprints” identifying specific human populations and used for phylogenetic analyses between species (Georgakopoulos-Soares et al. 2020).

As nullomers do not exist in a human genome, their appearance due to mutagenesis followed by clonal expansion could be exploited as a diagnostic method for diseases associated with a mutational burden, such as cancer.

### SUMMARY OF EMBODIMENTS

The disclosure relates to methods and compositions for the detection, identification, classification and characterization of cancer in general and cancer types in biological material.

The disclosure provides a method of identifying one or a plurality of nullomers in a sample comprising: (a) isolating a plurality of nucleic acids from the sample; (b) contacting the nucleic acids to one or a plurality of probes specific for one or a plurality of nullomers; (c) detecting the presence of the probes associated with the one or plurality of nullomers; and (d) correlating the presence or quantity of probes with the likelihood of the presence or quantity of nullomers in the sample. In some embodiments, the one or plurality of probes comprise a complementary nucleic acid sequence bound to or associated with a fluorescent molecule, radioactive isotope or chemiluminescent molecule. In some embodiments, the step of detecting is performed by mass spectrometry.

In some embodiments, the method further comprises, prior to step (b), disassociating a plurality of double stranded nucleic acid sequences comprising at least one nullomer by exposing the double-stranded nucleic acid sequences to a predetermined melting temperature for a period of time sufficient to create single stranded nullomer, annealing at least one primer to the nullomer, and allowing a sufficient period of time to extend the primer in the presence of dNTPs and DNA polymerase. In some embodiments, the steps of disassociating a plurality of double stranded nucleic acid sequences comprising at least one nullomer by exposing the double-stranded nucleic acid sequences to a predetermined melting temperature for a period of time sufficient to create single stranded nullomer, annealing at least one primer to the nullomer, and allowing a sufficient period of time to extend the primer in the presence of dNTPs and polymerase are repeated multiple times such that copies of the at least one nullomer are produced.

The disclosure further provides a method of identifying one or plurality of nullomers in a sample comprising: (a) isolating a plurality of nucleic acids from the sample; (b) contacting the nucleic acids to one or a plurality of probes specific for one or a plurality of nullomers; (c) detecting the presence of the probes associated with the one or plurality of nullomers; (d) correlating the presence or quantity of probes with the likelihood or the presence or quantity of nullomers in the sample; and (e) comparing the sequence of the nullomer with the sequence of a library of known nullomer sequences. In some embodiments, the probe or plurality of probes comprise a complementary nucleic acid sequence bound to or associated with a fluorescent molecule, radioactive isotope or chemiluminescent molecule. In some embodiments, the method further comprises a step of performing polymerase chain reaction (PCR) with one or a plurality of primers specific for the one or plurality of nullomers.

The disclosure also provides a computer-implemented method of identifying a mutation associated with a hyperproliferative disorder comprising: (a) isolating one or a plurality of nucleic acid molecules from a sample associated with the hyperproliferative disorder; (b) contacting the nucleic acids to one or a plurality of probes specific for one or a plurality of nullomers; (c) in a system configured to compile data and detect the presence or quantify the presence of a nucleic acid sequence, detecting the presence of the probes associated with the one or plurality of nullomers; (d) correlating the presence or quantity of the nullomer to the likelihood of a specific mutation serving as a biomarker for a hyperproliferative disorder. In some embodiments, the method further comprises, prior to step (a), in a system configured to compile data and detect the presence or quantity of nucleic acids in a sample: compiling genetic data about a population of subjects including the subject that has a mutation candidate that is a biomarker for a hyperproliferative disorder. In some embodiments, the method further comprises, after step (d), a step of: (e) selecting a cancer treatment for the subject based upon identification of the hyperproliferative disorder. In some embodiments, the hyperproliferative disorder is breast cancer, pancreatic cancer, or liver cancer. In some embodiments, the hyperproliferative disorder is breast cancer, pancreatic cancer, esophagus cancer, lymphoid cancer, kidney cancer, ovary cancer, head and neck cancer, lung cancer, stomach cancer, CNS cancer, uterus cancer, skin cancer, colorectal cancer, prostate cancer, bladder cancer, bone and soft tissue cancer, biliary cancer, cervix cancer, thyroid cancer, myeloid cancer, or liver cancer. In some embodiments, the hyperproliferative disorder is a malignant tumor. In some embodiments, the sample is a brush biopsy, puncture biopsy, fluid from a needle biopsy, blood, blood cells, cells from a hair sample, nucleic acids from a hair sample, saliva, or spit. In some embodiments, the probe or plurality of probes comprise a complementary nucleic acid sequence bound to or associated with a fluorescent molecule, radioactive isotope or chemiluminescent molecule. In some embodiments, the method further comprises a step of performing PCR with one or a plurality of primers specific for the one or plurality of nullomers.

The disclosure additionally provides a method of treating a hyperproliferative disorder in a subject in need thereof comprising: (a) exposing a sample from the subject to a probe specific for at least one nullomer chosen from Table 1; (b) detecting the presence, absence or quantity of the at least one nullomer in the sample; (c) normalizing the presence, absence, or quantity of the at least one nullomer in the sample against the presence, absence or quantity of the at least one nullomer in a sample of a healthy subject or a sample of a subject known to have the hyperproliferative disorder; (d) correlating the presence, absence, or quantity of the at least one nullomer in the sample to the subject having the hyperproliferative disorder; and (e) administering a therapeutically effective amount of one or a plurality of active agents to the subject. In some embodiments, the method further comprises obtaining the sample from the subject prior to the step of exposing. In some embodiments, the one or plurality of active agents is chosen from one or a combination of the agents identified in Table 3. In some embodiments, the sample is plasma, serum, whole blood, respiratory tissue, respiratory mucosal sample, saliva, urine, blood cells, cells from a hair sample, nucleic acids from a hair sample, or spit. In some embodiments, step (b) further comprises calculating one or more scores based upon the presence, absence, or quantity of the at least one nullomer, and step (d) further comprises correlating the one or more scores to the presence, absence, or quantity of the at least one nullomer such that, if the amount of the at least one nullomer is greater than the quantity of the at least one nullomer in a control sample; or, if the amount of the at least one nullomer is substantially equal to the quantity of the at least one nullomer in a sample taken from a subject known to have a hyperproliferative disorder, then the subject is diagnosed as having a hyperprolifferative disorder. In some embodiments, the probe is a radioactive probe, a chemoluminescent probe, or a fluorescent probe. In some embodiments, the sample is free of cells.

In some embodiments, the at least one nullomer is detected by next generation sequencing, quantitative real-time reverse transcription-PCR (qRT-PCR), isothermal amplification, microarray, multiplex nullomer profiling assay, RNA in situ hybridization (RNA-ish), or northern blotting. In some embodiments, the at least one nullomer is detected by qRT-PCR. In some embodiments, the step of quantifying at least one quantity of the at least one nullomer in the sample comprises using a fluorescence and/or digital imaging.

In some embodiments, the step of analysing comprises detecting a presence, absence, or quantity of at least 2 different nullomers. In some embodiments, the step of analysing comprises detecting the presence, absence, or quantity of the at least one nullomer by PCR amplification using one or a plurality of primers specific for the at least one nullomer chosen from Table 1. In some embodiments, the step of analysing comprises detecting presence, absence, or quantity of the at least one nullomer by a probe comprising a nucleic acid sequence complementary to the nucleic acid sequence of the at least one nullomer.

The disclosure further provide a method of diagnosing a subject with cancer comprising: (a) contacting a plurality of nucleic acids from a sample to a system comprising a probe specific for one or a plurality of nullomers; and (b) detecting the presence of or quantifying the amount of one or more nucleic acids from the sample. In some embodiments, the method comprises detecting the presence, absence or quantity of one or a plurality of the nullomers provided in Table 1. In some embodiments, the method comprises detecting the presence, absence or quantity of nullomers that comprise at least 93% sequence identify to one or a plurality of the nullomers provided in Table 1. In some embodiments, the at least one nullomer is detected by qRT-PCR. In some embodiments, the at least one nullomer is detected by CRISPR diagnosis. In some embodiments, the at least one nullomer is detected by CRISPR diagnosis and Cas9, Cas12 or Cas13 protein is used.

In some embodiments, the method further comprises, after the step of detecting, normalizing the quantity of the probe as compared to a quantity of signal from a negative control. In some embodiments, the method further comprises, after the step of detecting, correlating the one or more scores to the presence, absence, or quantity of the at least one nullomer such that, if the amount of the at least one nullomer is greater than the quantity of the at least one nullomer in a control sample; or, if the amount of the at least one nullomer is substantially equal to the quantity of the at least one nullomer in a sample taken from a subject known to have a hyperproliferative disorder, then the subject is diagnosed as having a hyperprolifferative disorder. In some embodiments, the hyperproliferative disorder is breast cancer, pancreatic cancer, or liver cancer. In some embodiments, the hyperproliferative disorder is breast cancer, pancreatic cancer, esophagus cancer, lymphoid cancer, kidney cancer, ovary cancer, head and neck cancer, lung cancer, stomach cancer, CNS cancer, uterus cancer, skin cancer, colorectal cancer, prostate cancer, bladder cancer, bone and soft tissue cancer, biliary cancer, cervix cancer, thyroid cancer, myeloid cancer, or liver cancer.

Also provided is a kit comprising one or more probes or primers for detecting the presence, absence or quantity of one or a plurality of the nullomers provided in Table 1 or nullomers that comprise at least 93% sequence identify to one or a plurality of the nullomers provided in Table 1. In some embodiments, the one or more probes comprised in the disclosed kit comprise one or a combination of the nullomer sequences of Table 1 or complementary thereof.

Further provided is a computer program product encoded on a computer-readable storage medium, wherein the computer program product comprises instructions for: a) detecting the presence, absence or quantity of at least one nullomer in a sample of a subject; b) normalizing the presence, absence, or quantity of the at least one nullomer in the sample against the presence, absence or quantity of the at least one nullomer in a control sample; and c) correlating the presence, absence, or quantity of the at least one nullomer in the sample to a likelihood that the subject having a hyperproliferative disorder. In some embodiments, the computer program product further comprises instructions for calculating a score associated with the presence, absence or quantity of the at least one nullomer in the sample and correlating the score to a likelihood that the subject has a hyperproliferative disorder. In some embodiments, the computer program product further comprises instructions for: a) detecting and normalizing the presence, absence or quantity of a second nullomer in the sample; b) calculating a combined score associated with the presence, absence or quantity of the at least one nullomer and the second nullomer in the sample; and c) correlating the combined score to a likelihood that the subject having a hyperproliferative disorder. In some embodiments, at least 2 different nullomers in the sample are detected, normalized and correlated by the computer program product. In some embodiments, the computer program product detects the presence, absence, or quantity of the at least one nullomer by qRT-PCR amplification. In some embodiments, the control sample used in the computer program product is obtained from a subject free of a hyperproliferative disorder.

The disclosure also provides a system comprising: a) the computer program product of any one of claims 54 to 59; and b) a processor operable to execute programs; and/or a memory associated with the processor.

The disclosure further provides a system for detecting the presence or quantity of nullomer in a sample of a subject comprising: a processor operable to execute programs, a memory associated with the processor, a database associated with said processor and said memory, and a program stored in the memory and executable by the processor, the program being operable for: a) detecting the presence, absence or quantity of at least one nullomer in a sample of a subject; b) normalizing the presence, absence, or quantity of the at least one nullomer in the sample against the presence, absence or quantity of the at least one nullomer in a control sample; and c) correlating the presence, absence, or quantity of the at least one nullomer in the sample to a likelihood that the subject having a hyperproliferative disorder. In some embodiments, the program is further operable for calculating a score associated with the presence, absence or quantity of the at least one nullomer in the sample and correlating the score to a likelihood that the subject has a hyperproliferative disorder. In some embodiments, the program is further operable for detecting and normalizing the presence, absence or quantity of a second nullomer in the sample.

In some embodiments, the one or plurality of probes used in any of the disclosed methods, systems, or computer program product, or comprised in any of the disclosed kits comprise a nucleic acid sequence chosen from Table 1. In some embodiments, the one or plurality of probes used in any of the disclosed methods, systems, or computer program product, or comprised in any of the disclosed kits comprise a nucleic acid sequence comprising at least about 93% sequence identity to any of the sequences in Table 1.

In some embodiments, the one or plurality of probes used in any of the disclosed methods, systems, or computer program product, or comprised in any of the disclosed kits comprise a nucleic acid sequence that is complementary to any of the nullomer sequences provided in Table 1, or a fragment thereof. In some embodiments, the one or plurality of probes used in any of the disclosed methods, systems, or computer program product, or comprised in any of the disclosed kits comprise a nucleic acid sequence that is complementary to a nullomer comprising at least about 93% sequence identity to any of the nullomer sequences provided in Table 1, or a fragment thereof.

In some embodiments, the one or plurality of primers specific for the one or plurality of nullomers used in any of the disclosed methods, systems, or computer program product, or comprised in any of the disclosed kits comprise a nucleic acid sequence that is complementary to any of the nullomer sequences provided in Table 1, or a fragment thereof. In some embodiments, the one or plurality of primers specific for the one or plurality of nullomers used in any of the disclosed methods, systems, or computer program product, or comprised in any of the disclosed kits comprise a nucleic acid sequence that is complementary to a nullomer comprising at least about 93% sequence identity to any of the nullomer sequences provided in Table 1, or a fragment thereof.

### BRIEF DESCRIPTION OF DRAWINGS

FIG. 1A-1E depict nullomers in the PCAWG dataset. FIG. 1A: Schematic overview of our pipeline for identifying nullomers and using them to distinguish and detect tumors. FIG. 1B: Association between number of mutations and number of resurfaced nullomers observed. FIG. 1C: Number of non-redundant recurrent nullomers and the number of substitutions for X patients (Spearman's rho=X). FIG. 1D: Overlap of recurrent nullomers for each cancer type. The heatmap shows the Jaccard index for the amount of overlap for nullomer sets associated with different cancer types. FIG. 1E: Heatmap showing the occurrence of the recurrent nullomers across patients. Each row represents a patient and the intensity of the heatmap (log 2-scale) shows the number of nullomers from each tissue set.

FIG. 2A-2C depict classification of and detection of tumors based on nullomers. FIG. 2A: Accuracy of classifier. FIG. 2B: Confusion matrix. FIG. 2C: Results for 6 prostate cancer patients and 23 healthy controls profiled using WGS.

FIG. 3A-3C depict nullomer promoter assays. FIG. 3A-3B: UCSC Genome Browser snapshots of the RPS2 (FIG. 3A) and TMEM127 (FIG. 3B) loci showing the promoter (dark rectangle) and nullomer (grey dot) locations. FIG. 3C: Luciferase reporter assays comparing reference (WT) and nullomer encompassing sequence (NUL). POS=positive control, NEG=negative control, *=p-value<0.05 and ***=p-value<0.001 for a Student T-test.

FIG. 4 depicts a flowchart outlining steps for identification of nullomers.

### DETAILED DESCRIPTION OF EMBODIMENTS

Before the present methods and systems are described, it is to be understood that the present disclosure is not limited to the particular processes, compositions, or methodologies described, as these may vary. It is also to be understood that the terminology used in the description is for the purposes of describing the particular versions or embodiments only, and is not intended to limit the scope of the present disclosure. Unless defined otherwise, all technical and scientific terms used herein have the same meanings as commonly understood by one of ordinary skill in the art. Although any methods and materials similar or equivalent to those described herein can be used in the practice or testing of embodiments of the present disclosure, the methods, devices, and materials in some embodiments are now described. All publications mentioned herein are incorporated by reference in their entireties. Nothing herein is to be construed as an admission that the present disclosure is not entitled to antedate such disclosure by virtue of prior invention.

### Definitions

Unless specifically defined otherwise, all technical and scientific terms used herein shall be taken to have the same meaning as commonly understood by one of ordinary skill in the art (e.g., in cell culture, molecular genetics, microRNA and detection thereof, immunology, immunohistochemistry, protein chemistry, and biochemistry). The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of.” “Consisting essentially of,” when used in the claims, shall have its ordinary meaning as used in the field of patent law.

The term “about” is used herein to mean within the typical ranges of tolerances in the art. For example, “about” can be understood as about 2 standard deviations from the mean. According to certain embodiments, when referring to a measurable value such as an amount and the like, “about” is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value as such variations are appropriate to perform the disclosed methods. When “about” is present before a series of numbers or a range, it is understood that “about” can modify each of the numbers in the series or range.

As used herein, the term “animal” includes, but is not limited to, humans and non-human vertebrates such as wild animals, rodents, such as rats, ferrets, and domesticated animals, and farm animals, such as dogs, cats, horses, pigs, cows, sheep, and goats. In some embodiments, the animal is a mammal. In some embodiments, the animal is a human. In some embodiments, the animal is a non-human mammal.

An “algorithm,” “formula,” or “model” is any mathematical equation, algorithmic, analytical or programmed process, or statistical technique that takes one or more continuous or categorical inputs (herein called “parameters”) and calculates an output value, sometimes referred to as an “index” or “index value.” Non-limiting examples of “formulas” include sums, ratios, and regression operators, such as coefficients or exponents, biomarker (e.g., nullomers disclosed herein) value transformations and normalizations (including, without limitation, those normalization schemes based on clinical parameters, such as gender, age, or ethnicity), rules and guidelines, statistical classification models, and neural networks trained on historical populations. Of particular use in combining markers are linear and non-linear equations and statistical classification analyses to determine the relationship between levels of the biomarkers detected in a subject sample and the subject's risk of disease (for example). In panel and combination construction, of particular interest are structural and syntactic statistical classification algorithms, and methods of risk index construction, utilizing pattern recognition features, including established techniques such as cross correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shruken Centroids (SC), StepAIC, Kth-Nearest Neighbor, Boosting, Decision Trees, Neural Networks, Bayesion Networks, Support Vector Machines, and Hidden Markov Models, among others. Many of these techniques are useful either combined with a biomarker selection technique, such as forward selection, backwards selection, or stepwise selection, complete enumeration of all potential panels of a given size, genetic algorithms, or they may themselves include biomarker selection methodologies in their own technique. These may be coupled with information criteria, such as Akaike's Information Criterion (AIC) or Bayes Information Criterion (BIC), in order to quantify the tradeoff between additional biomarkers and model improvement, and to aid in minimizing overfit. The resulting predictive models may be validated in other studies, or cross-validated in the study they were originally trained in, using such techniques as Leave-One-Out (LOO) and 10-Fold cross-validation (10-Fold-CV).

The term “at least” prior to a number or series of numbers (e.g. “at least two”) is understood to include the number adjacent to the term “at least,” and all subsequent numbers or integers that could logically be included, as clear from context. When “at least” is present before a series of numbers or a range, it is understood that “at least” can modify each of the numbers in the series or range.

The term “biomarker” as used herein refers to a biological molecule present in an individual at varying concentrations useful in predicting the cancer status of an individual. A biomarker may include but is not limited to, nucleic acids, proteins and variants and fragments thereof. A biomarker may be DNA comprising the entire or partial nucleic acid sequence encoding the biomarker, or the complement of such a sequence. Biomarker nucleic acids useful in the disclosure are considered to include both DNA and RNA comprising the entire or partial sequence of any of the nucleic acid sequences of interest. In some embodiments, the biomarker of the disclosure is any of the nullomers disclosed herein.

The term “bodily fluid” as used herein refers to a bodily fluid including blood (or a fraction of blood such as plasma or serum), lymph, mucus, tears, saliva, sweat, sputum, urine, semen, stool, cerebrospinal fluid (CSF), breast milk, and, ascities fluid. In some embodiments, the bodily fluid is blood. In some embodiments, the bodily fluid is a fraction of blood. In some embodiments, the bodily fluid is plasma. In some embodiments, the bodily fluid is serum. In some embodiments, the bodily fluid is urine.

The terms “cancer” and “cancerous” as used herein refer to or describe a physiological condition in mammals in which a population of cells are characterized by unregulated cell growth. Thus, the term “cancer” refers to a group of diseases involving abnormal cell growth with the potential to invade or spread to other parts of the body. Examples of cancer include, but not limited to, lung cancer, bone cancer, blood cancer, chronic myelomonocytic leukemia (CMML), bile duct cancer, cervical cancer, liver cancer, pancreatic cancer, skin cancer, cancer of the head and neck, cancer of the eye, cutaneous or intraocular melanoma, uterine cancer, ovarian cancer, rectal cancer, cancer of the anal region, stomach cancer, colon cancer, breast cancer, testicular cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's disease, cancer of the esophagus, cancer of the small intestine, cancer of the endocrine system (e.g., cancer of the thyroid, parathyroid or adrenal glands), sarcomas of soft tissues, cancer of the urethra, cancer of the penis, prostate cancer, chronic or acute leukemia, solid tumors of childhood, lymphocytic lymphomas, cancer of the bladder, cancer of the kidney or ureter (e.g., renal cell carcinoma, carcinoma of the renal pelvis), or neoplasms of the central nervous system (e.g., primary CNS lymphoma, spinal axis tumors, brain stem gliomas or pituitary adenomas).

As used herein, the term “characterizing cancer in a subject” refers to the identification of one or more properties of a cancer sample in a subject, including but not limited to, the presence of benign, pre-cancerous or cancerous tissue, the stage of the cancer, the type of the cancer, the tissue of origin of the cancer, and the subject's prognosis. Cancers may be characterized by the identification of the expression of one or more cancer marker genes, including but not limited to, the nullomers disclosed herein. As used herein, the term “stage of cancer” refers to a qualitative or quantitative assessment of the level of advancement of a cancer. Criteria used to determine the stage of a cancer include, but are not limited to, the size of the tumor and the extent of metastases (e.g., localized or distant). In some embodiments, the subject has been previously diagnosed with having a cancer and received, or is currently receiving, cancer treatment, including but not limited to surgical intervention and cancer therapy, and in such embodiments, the term “characterizing cancer in a subject” refers to monitoring the progress of the cancer treatment.

The terms “complementary” or “complementarity” refer to polynucleotides (i.e., a sequence of nucleotides) related by base-pairing rules, for example, the sequence “5′-AGT-3′,” is complementary to the sequence “5′-ACT-3′.” Complementarity may be “partial,” in which only some of the nucleic acids' bases are matched according to the base pairing rules, or there may be “complete” or “total” complementarity between the nucleic acids. The degree of complementarity between nucleic acid strands can have significant effects on the efficiency and strength of hybridization between nucleic acid strands under defined conditions. This is of particular importance for methods that depend upon binding between nucleic acid bases.

As used herein, the terms “comprising” (and any form of comprising, such as “comprise,” “comprises,” and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

The term “correlate” or “correlating” as used herein refers to a statistical association between instances of two events, where events may include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases. The disclosure provides nullomers, the levels of which are correlated with a particular outcome measure, such as between the presence of a particular nullomer and the likelihood of developing a particular type of cancer. For example, the increased level of a nullomer may be negatively correlated with a likelihood of good clinical outcome for the patient. In this case, for example, the patient may have a decreased likelihood of long-term survival without recurrence of the cancer and/or a positive response to a chemotherapy, and the like. Such a negative correlation indicates that the patient likely has a poor prognosis or will respond poorly to a chemotherapy, and this may be demonstrated statistically in various ways, e.g., by a high hazard ratio.

As used herein, the terms “detect,” “detecting” or “detection” refer to either the general act of discovering or discerning or the specific observation of a composition. Detecting a composition may comprise determining the presence or absence of a composition. Detecting may comprise quantifying a composition. For example, detecting comprises determining the expression level of a composition. The composition may comprise a nucleic acid molecule. For example, the composition may comprise one or a plurality of the nullomers disclosed herein. Alternatively, or additionally, the composition may be a detectably labeled composition.

The term “diagnosis” or “prognosis” as used herein refers to the use of information (e.g., genetic information or data from other molecular tests on biological samples, signs and symptoms, physical exam findings, cognitive performance results, etc.) to anticipate the most likely outcomes, timeframes, and/or response to a particular treatment for a given disease, disorder, or condition, based on comparisons with a plurality of individuals sharing common nucleotide sequences, symptoms, signs, family histories, or other data relevant to consideration of a patient's health status.

The terms “functional fragment” means any portion of a polypeptide or nucleic acid sequence from which the respective full-length polypeptide or nucleic acid relates that is of a sufficient length and has a sufficient structure to confer a biological affect that is similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full-length or wild-type protein. In some embodiments, the functional fragment may have a reduced biological activity, about equivalent biological activity, or an enhanced biological activity as compared to the wild-type or full-length polypeptide sequence upon which the fragment is based. In some embodiments, the functional fragment is derived from the sequence of an organism, such as a human. In such embodiments, the functional fragment may retain about 99%, 98%, 97%, 96%, 95%, 94%, 93%, 92%, 91%, or 90% sequence identity to the wild-type or given sequence upon which the sequence is derived. In some embodiments, the functional fragment may retain about 85%, 80%, 75%, 70%, 65%, or 60% sequence identity to the wild-type sequence upon which the sequence is derived. In some embodiments, the given sequence is a nullomer sequence of Table 1. In other embodiments, the given sequence is a complementary sequence of any of the nullomer sequences of Table 1.

The term “hyperproliferation” as used herein is defined as clonal expansion, in which daughter cells share a set of somatic mutations that were not originally present in the germline and which could include but are not limited to driver mutations. Clonal expansion could include but is not limited to resistance to cell death, evasion of growth suppressors, sustaining proliferate signaling, enabling replicative immortality, activating invasion and metastasis or inducing angiogenesis.

The term “hyperproliferative cell” refers to a cell located in a tissue or organ having a “hyperproliferative disorder,” a disease or disorder characterized by abnormal proliferation, abnormal growth, abnormal senescence, abnormal quiescence, or abnormal removal of cells in an organism, and includes all forms of hyperplasias, neoplasias, and cancer. In some embodiments, the “hyperproliferative cell” is a precancerous cell in form of hyperplasias. In some embodiments, the “hyperproliferative cell” is precancerous cell in form of neoplasias. In some embodiments, the “hyperproliferative cell” is a cancerous cell. In some embodiments, the hyperproliferative disorder or disease is a cancer derived from the gastrointestinal tract or urinary system. In some embodiments, a hyperproliferative disorder or disease is a cancer of the adrenal gland, bile ducts, bladder, blood, bone, bone marrow, brain, breast, cervix, colon, esophagus, eye, gall bladder, ganglia, gastrointestinal tract, heart, lymphatic system, liver, lung, kidney, muscle, ovary, pancreas, parathyroid, penis, prostate, prostate glands, rectum, salivary glands, skin, spine, stomach, spleen, testis, thymus, thyroid, or uterus. In some embodiments, the term hyperproliferative disorder or disease is a cancer chosen from: lung cancer, bone cancer, blood cancer, chronic myelomonocytic leukemia (CMML), bile duct cancer, cervical cancer, liver cancer, pancreatic cancer, skin cancer, cancer of the head and neck, cancer of the eye, cutaneous or intraocular melanoma, uterine cancer, ovarian cancer, rectal cancer, cancer of the anal region, stomach cancer, colon cancer, breast cancer, testicular cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's disease, cancer of the esophagus, cancer of the small intestine, cancer of the endocrine system (e.g., cancer of the thyroid, parathyroid or adrenal glands), sarcomas of soft tissues, cancer of the urethra, cancer of the penis, prostate cancer, chronic or acute leukemia, solid tumors of childhood, lymphocytic lymphomas, cancer of the bladder, cancer of the kidney or ureter (e.g., renal cell carcinoma, carcinoma of the renal pelvis), or neoplasms of the central nervous system (e.g., primary CNS lymphoma, spinal axis tumors, brain stem gliomas or pituitary adenomas). In some embodiments, the hyperproliferative disorder or disease is a breast cancer, pancreatic cancer, esophagus cancer, lymphoid cancer, kidney cancer, ovary cancer, head and neck cancer, lung cancer, stomach cancer, CNS cancer, uterus cancer, skin cancer, colorectal cancer, prostate cancer, bladder cancer, bone and soft tissue cancer, biliary cancer, cervix cancer, thyroid cancer, myeloid cancer, or liver cancer. In some embodiments, the hyperproliferative disorder or disease comprises one or a plurality of mutations in one or a plurality of genes selected from Table A.

TABLE A
Cancer-related genes and their corresponding GenBank accession numbers.

| Gene | GenBank | Gene | GenBank |
|---|---|---|---|
| TP53 | P04637.4 | MUC17 | Q685J3.2 |
| PIK3CA | P42336.2 | SPOP | O43791.1 |
| APC | P25054.2 | NAV3 | Q8IVL0.3 |
| KRAS | P01116.1 | FLG | P20930.3 |
| ARID1A | O14497.3 | TTN | Q8WZ42.4 |
| BRAF | P15056.4 | LRP1B | Q9NZR2.2 |
| PTEN | P60484.1 | HMCN1 | Q96RW7.2 |
| IDH1 | O75874.2 | KMT2C | Q8NEZ4.3 |
| CDKN2A | Q8N726.2 | CDKN2B | P42772.1 |
| MSH6 | P52701.2 | MYC | P01106.1 |
| RB1 | P06400.2 | SOX2 | P48431.1 |
| CTNNB1 | P35222.1 | MCL1 | Q07820.3 |
| ATM | Q13315.4 | HRAS | P01112.1 |
| FAT1 | Q14517.2 | BRCA1 | P38398.2 |
| TP63 | Q9H3D4.1 | BRCA2 | P51587.3 |
| FBXW7 | Q969H0.1 | PALB2 | Q86YC2.1 |
| FAT4 | Q6V017.2 | RAD51D | O75771.1 |
| BAP1 | Q92560.2 | TERT | O14746.1 |
| RPL22 | P35268.2 | RECQL | P46063.3 |
| TSHZ3 | Q63HK5.2 | CHEK2 | O96017.1 |
| RUNX1 | Q01196.3 | SMAD4 | Q13485.1 |
| NRAS | P01111.1 | MSH2 | P43246.1 |
| EGFR | P00533.2 | MUTYH | Q9UIF7.1 |
| PIK3R1 | P27986.2 | HNF1A | P20823.2 |
| NPM1 | P06748.2 | KIT | P10721.1 |

As used herein, the phrase “in need thereof” means that the animal or mammal has been identified or suspected as having a need for the particular method or treatment. In some embodiments, the identification can be by any means of diagnosis or observation. In any of the methods and treatments described herein, the animal or mammal can be in need thereof.

The term “label” as used herein refers to any atom or molecule that can be used to provide a detectable (preferably quantifiable) effect, and that can be attached to a nucleic acid or protein. Labels include but are not limited to dyes; radiolabels such as 2P; binding moieties such as biotin; haptens such as digoxgenin; luminogenic, phosphorescent or fluorogenic moieties; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, and the like. A label may be a charged moiety (positive or negative charge) or alternatively, may be charge neutral. Labels can include or consist of nucleic acid or protein sequence, so long as the sequence comprising the label is detectable. In some embodiments, nucleic acids are detected directly without a label (e.g., directly reading a sequence).

The term “level” as used herein refers to qualitative or quantitative amount of the number of copies of a nullomer. A nullomer exhibits an “increased level” when the level of the nullomer is higher in a first sample, such as in a clinically relevant subpopulation of patients (e.g., patients who have cancer), than in a second control sample, such as in a related subpopulation (e.g., patients who do not have cancer). In the context of an analysis of a level of a nullomer in a tumor sample obtained from an individual patient, a nullomer exhibits “increased level” when the level of the nullomer in the subject trends toward, or more closely approximates, the level characteristic of a clinically relevant subpopulation of patients.

The term “measuring” or “measurement” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters. Alternatively, the term “detecting” or “detection” may be used and is understood to cover all measuring or measurement as described herein.

The term “metastasis” as used herein refers to the process by which a cancer spreads or transfers from the site of origin to other regions of the body. A “metastatic” or “metastasizing” cell is one that loses adhesive contacts with neighboring cells and migrates (e.g., via the bloodstream or lymph) from the primary site of disease to secondary sites.

The particular use of terms “nucleic acid,” “oligonucleotide,” and “polynucleotide” should in no way be considered limiting and may be used interchangeably herein. “Oligonucleotide” is used when the relevant nucleic acid molecules typically comprise less than about 100 bases. “Polynucleotide” is used when the relevant nucleic acid molecules typically comprise more than about 100 bases. Both terms are used to denote a DNA, RNA, modified or synthetic DNA or RNA sequence (including, but not limited to nucleic acids comprising synthetic and naturally-occurring base analogs, dideoxy or other sugars, thiols or other non-natural or natural polymer backbones), or other nucleobase containing polymers capable of hybridizing to DNA and/or RNA. Accordingly, the terms should not be construed to define or limit the length of the nucleic acids referred to and used herein, nor should the terms be used to limit the nature of the polymer backbone to which the nucleobases are attached.

The term “nucleic acid sequence” or “polynucleotide sequence” refers to a contiguous string of nucleotide bases and in particular contexts also refers to the particular placement of nucleotide bases in relation to each other as they appear in a polynucleotide.

“Nucleobase” means a heterocyclic moiety capable of non-covalently pairing with another nucleobase.

“Nucleoside” means a nucleobase linked to a sugar moiety.

“Nucleotide” means a nucleoside having a phosphate group covalently linked to the sugar portion of a nucleoside. In some embodiments, the nucleotide is characterized as being modified if the 3′ phosphate group is covalently linked to a contiguous nucleotide by any linkage other than a phosphodiester bond.

“Compound comprising a modified oligonucleotide consisting of a number of linked nucleosides” means a compound that includes a modified oligonucleotide having the specified number of linked nucleosides. Thus, the compound may include additional substituents or conjugates. Unless otherwise indicated, the compound does not include any additional nucleosides beyond those of the modified oligonucleotide.

“Modified oligonucleotide” means an oligonucleotide having one or more modifications relative to a naturally occurring terminus, sugar, nucleobase, and/or internucleoside linkage. A modified oligonucleotide may comprise unmodified nucleosides.

“Single-stranded modified oligonucleotide” means a modified oligonucleotide which is not hybridized to a complementary nucleic acid strand.

“Modified nucleoside” means a nucleoside having any change from a naturally occurring nucleoside. A modified nucleoside may have a modified sugar, and an unmodified nucleobase. A modified nucleoside may have a modified sugar and a modified nucleobase. A modified nucleoside may have a natural sugar and a modified nucleobase. In some embodiments, a modified nucleoside is a bicyclic nucleoside. In some embodiments, a modified nucleoside is a non-bicyclic nucleoside.

The term “nullomers” as used herein refers to expressed oligonucleotide sequences in a species, the genetic templates of which are congenitally absent in the species. In some embodiments, the nullomers of the disclosure are nullomers not present in the published human genome sequences. In some embodiments, the nullomers of the disclosure are nullomers not present in the published human genome sequences and associated with one or a plurality of cancers.

As used herein “one or more of” includes at least one of the recited components, or 2, 3, 4, 5, or 5 etc. of the recited components. In some embodiments, the phase includes all of the recited components.

Ranges provided herein are understood to include all individual integer values and all subranges within the ranges.

As used herein, the term “sample” refers to a biological sample obtained or derived from a source of interest, as described herein. In some embodiments, a source of interest comprises an organism, such as an animal or human. In some embodiments, a biological sample comprises biological tissue or fluid. In some embodiments, a biological sample may be or comprise bone marrow, blood, blood cells, cells from a hair sample, ascites, tissue or fine needle biopsy samples, cell-containing body fluids, free floating nucleic acids, sputum, saliva or spit, urine, cerebrospinal fluid, peritoneal fluid, pleural fluid, feces, lymph, gynecological fluids, skin swabs, vaginal swabs, oral swabs, nasal swabs, washings or lavages such as a ductal lavages or broncheoalveolar lavages, aspirates, scrapings, bone marrow specimens, tissue biopsy specimens, surgical specimens, feces, other body fluids, secretions and/or excretions, and/or cells therefrom, etc. In some embodiments, the sample is a brush biopsy, puncture biopsy, or fluid from a needle biopsy. In some embodiments, the sample is blood or blood cells. In some embodiments, the sample is cells from a hair sample or nucleic acids from a hair sample. In some embodiments, the sample is sputum, saliva or spit. In some embodiments, a biological sample is or comprises cells obtained from an individual. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

As used herein, the term “minimal residual disease” refers to a small number of cancer cells remaining in the body after treatment or surgical intervention. These cells cannot usually be detected by standard scans or tests, due to lower abundance than detection sensitivity thresholds.

A “score” is a value or set of values selected so as to provide a normalized quantitative measure of a variable or characteristic of a subject's condition, and/or to discriminate, differentiate or otherwise characterize a subject's condition. The value(s) comprising the score can be based on, for example, quantitative data resulting in a measured amount of one or more sample constituents obtained from the subject, or from clinical parameters, or from clinical assessments, or any combination thereof. In certain embodiments, the score can be derived from a single constituent, parameter or assessment, while in other embodiments the score is derived from multiple constituents, parameters and/or assessments. The score can be based upon or derived from an interpretation function; e.g., an interpretation function derived from a particular predictive model using any of various statistical algorithms known in the art. A “change in score” can refer to the absolute change in score, e.g. from one time point to the next, or the percent change in score, or the change in the score per unit time (i.e., the rate of score change). In some embodiments, the score is calculated through an interpretation function or algorithm. In some embodiments, the subject is suspected of having expression of a gene that promotes or contributes to the likelihood of acquiring a disease state or whose expression is correlative to the presence of a pathogen. Calculation of score can be accomplished using known algorithms executable in computer program products within equipment used in sequencing or analyzing samples. In some embodiments, the methods disclosed herein comprise substeps of detecting the presence, absence or quantity of a given biomarker by calculating the quantity of a probe in a control sample, calculating the quantity of a probe in the subject sample, and normalizing the signal obtained from the subject sample by subtracting the signal obtained from the control sample.

As used herein, “sequence identity” is determined by using the stand-alone executable BLAST engine program for blasting two sequences (b12seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). Alternatively, “% sequence identity” can be determined using the EMBOSS Pairwise Alignment Algorithms tool available from The European Bioinformatics Institute (EMBL-EBI), which is part of the European Molecular Biology Laboratory (EMBL). This tool is accessible at the website ebi.ac.uk/Tools/emboss/align/. This tool utilizes the Needleman-Wunsch global alignment algorithm (Needleman, S. B. and Wunsch, C. D. (1970) J. Mol. Biol. 48, 443-453; Kruskal, J. B. (1983) An overview of sequence comparison, In D. Sankoff and B. Kruskal, (ed.), Time warps, string edits and macromolecules: the theory and practice of sequence comparison, pp. 1-44, Addison Wesley). Default settings are utilized which include Gap Open: 10.0 and Gap Extend 0.5. The default matrix “Blosum62” is utilized for amino acid sequences and the default matrix “DNAfull” is utilized for nucleic acid sequences.

As used herein, the term “statistically significant” means an observed alteration is greater than what would be expected to occur by chance alone (e.g., a “false positive”). Statistical significance can be determined by any of various methods well-known in the art. An example of a commonly used measure of statistical significance is the p-value. The p-value represents the probability of obtaining a given result equivalent to a particular datapoint, where the datapoint is the result of random chance alone. A result is often considered highly significant (not random chance) at a p-value less than or equal to about 0.05.

The terms “subject,” “individual,” and “patient” are used interchangeably herein to refer to a vertebrate, preferably a mammal, more preferably a human. Mammals include, but are not limited to, murine, simians, humans, farm animals, cows, pigs, goats, sheep, horses, dogs, sport animals, and pets. Tissues, cells and their progeny obtained in vivo or cultured in vitro are also encompassed by the definition of the term “subject.” In some embodiments, the subject is a human. For treatment of those conditions which are specific for a specific subject, such as a human being, the term “patient” may be interchangeably used. In some instances in the description of the present disclosure, the term “patient” will refer to human patients suffering from a particular disease or disorder. In some embodiments, the subject may be a non-human animal. The term “mammal” encompasses both humans and non-humans and includes but is not limited to humans, non-human primates, canines, felines, murine, bovines, equines, caprine, and porcines.

By “substantially identical” is meant a nucleic acid molecule (or polypeptide) comprises at least about 50% sequence identity to a reference nucleic acid sequence (for example, any one of the nucleic acid sequences described herein) or amino acid sequence. In some embodiments, such a sequence is at least about 60%, 70%, 80%, 85%, 90%, 91%, 92%, 93%, 94%, 95%, or even 99% identical at the nucleic acid level or amino acid level to the reference sequence used for comparison.

As used herein, the term “therapeutic” means an agent utilized to treat, combat, ameliorate, prevent or improve an unwanted condition or disease of a patient.

The term “therapeutically effective amount” means a quantity sufficient to achieve a desired therapeutic effect, for example, an amount which results in the prevention or amelioration of or a decrease in the symptoms associated with a disease that is being treated, e.g., disorders associated with cancer growth or a hyperproliferative disorder. The amount of compound administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The regimen of administration can affect what constitutes an effective amount. Further, several divided dosages, as well as staggered dosages, can be administered daily or sequentially, or the dose can be continuously infused, or can be a bolus injection. Further, the dosages of the compound(s) of the disclosure can be proportionally increased or decreased as indicated by the exigencies of the therapeutic or prophylactic situation. Typically, an effective amount of the compounds of the present disclosure, sufficient for achieving a therapeutic effect, range from about 0.000001 mg per kilogram body weight per day to about 10,000 mg per kilogram body weight per day. Preferably, the dosage ranges are from about 0.0001 mg per kilogram body weight per day to about 100 mg per kilogram body weight per day. The compounds disclosed herein can also be administered in combination with each other, or with one or more additional therapeutic compounds.

The terms “treatment” or “treating” as used herein is an approach for obtaining beneficial or desired results including clinical results for the subject. For purposes herein, beneficial or desired clinical results include, but are not limited to, one or more of the following: (1) preventing or delaying the appearance of clinical symptoms of the state, disorder, or condition developing in a person who may be afflicted with or predisposed to the state, disorder or condition but does not yet experience or display clinical symptoms of the state, disorder or condition; (2) inhibiting the state, disorder or condition, i.e., arresting, reducing or delaying the development of the disease or a relapse thereof (in case of maintenance treatment) or at least one clinical symptom, sign, or test, thereof; or (3) relieving the disease, i.e., causing regression of the state, disorder or condition or at least one of its clinical or sub-clinical symptoms or signs. In some embodiments, a subject is successfully “treated” according to the methods of the present disclosure if the patient shows one or more of the following: a reduction in the number of and/or complete absence of cancer cells; a reduction in the tumor size; an inhibition of tumor growth; inhibition of and/or an absence of cancer cell infiltration into peripheral organs including the spread of cancer cells into soft tissue and bone; inhibition of and/or an absence of tumor or cancer cell metastasis; inhibition and/or an absence of cancer growth; relief of one or more symptoms associated with the specific cancer; reduced morbidity and mortality; improvement in quality of life; reduction in tumorigenicity; reduction in the number or frequency of cancer stem cells; or some combination of such effects.

The term “tumor” as used herein, refers to all neoplastic cell growth and proliferation, whether malignant or benign, and all pre-cancerous and cancerous cells and tissues. A “benign” tumor is not cancerous and it does not invade nearby tissue or spread to other parts of the body. A “premalignant” tumor is a tumor which is not yet cancerous but has the potential to become malignant. A “malignant” tumor, on the other hand, is cancerous and can grow and spread to other parts of the body.

The term “tumor sample” as used herein refers to a sample comprising tumor material obtained from a cancer patient. The term encompasses tumor tissue samples, for example, tissue obtained by surgical resection and tissue obtained by biopsy, such as for example, a core biopsy or a fine needle biopsy. In some embodiments, the tumor sample is a fixed, wax-embedded tissue sample, such as a formalin-fixed, paraffin-embedded tissue sample. Additionally, the term “tumor sample” encompasses a sample comprising tumor cells obtained from sites other than the primary tumor, e.g., circulating tumor cells. The term also encompasses cells that are the progeny of the patient's tumor cells, e.g. cell culture samples derived from primary tumor cells or circulating tumor cells. The term further encompasses samples that may comprise protein or nucleic acid material shed from tumor cells in vivo, e.g., bone marrow, blood, plasma, serum, and the like. The term also encompasses samples that have been enriched for tumor cells or otherwise manipulated after their procurement and samples comprising polynucleotides and/or polypeptides that are obtained from a patient's tumor material.

### Identification of Nullomers

The identification of nullomers can be performed using any methods known in the art. In some embodiments, the identification of nullomers of the disclosure is performed as previously described in Georgakopoulos-Soares et al., published in bioRxiv, available at biorxiv.org/content/10.1101/2020.03.02.972422v1, incorporated by reference herein. As a first step, a dataset is obtained. In some embodiments, the dataset is obtained from WGS cancers from ICGC under the project PanCancer Analysis of Whole Genomes (ICGC/TCGA Pan-Cancer Analysis of Whole Genomes Consortium. Pan-cancer analysis of whole genomes, Nature, 2020, 578:82-93), which includes 46 cancer projects from 21 organs. WGS patients were analyzed using the GRCh37 (hg19) reference assembly of the human genome.

In some embodiments, somatic indel calls are performed using three pipelines from four somatic variant callers. These are the Wellcome Sanger Institute pipeline, the DKFZ/EMBL pipeline and the Broad Institute pipeline, with somatic variant false discovery rate of about 2.5%. In some embodiments, indel calling is performed by those algorithms and only indels called by at least two of the callers were analyzed, therefore generating a conservative dataset. As a result, the false negative rate of indel detection can be higher than that of other methods, and of each pipeline separately, which implies that many indels present in the samples were not identified successfully. For a small subset of indels, in some embodiments, the indel calls are visually examined using JBrowse Genome Browser32, to inspect the number of reads reporting the indel, if the indel calls are biased towards the end of the sequencing reads or if there were other systematic biases between the normal and tumor sequencing reads; such biases could not be identified.

In some embodiments, Bedtools intersect utility is used to measure overlap between indels and polyN tracts. The term overlap in this context refers to deleted bases occurring at any position across the entire length of the repeat or inserted bases occurring at any position across the length of the repeat and immediately before or after the repeat. Indel density is defined as the number of indel mutations for a given number of bases.

In some embodiments, the distance between each pair of consecutive indels is calculated per patient. In some embodiments, indels in different chromosomes are excluded because their pairwise distance cannot be defined. In some embodiments, the same analysis is performed separately for insertions and deletions.

In some embodiments, substitution calling is performed using four somatic mutation-calling algorithms, with mutation calls being shared by at least two algorithms. In the embodiments for lung cancers, C>A substitutions can be examined with respect to transcriptional strand asymmetries at polyG tracts and replication timing.

In some embodiments, the numbers of indels overlapping motifs found in the template or non-template strands are obtained using the bedtools intersect command. In some embodiments, strand bias is calculated for the vector of genes, reporting the number of polyN motif occurrences and the number of overlapping motifs as:
A=(indels overlapping motif at non-template)/(motif occurrences at non-template)
B=(indels overlapping motif at template)/(motif occurrences at template)
Strand bias=A/(A+B)
with motifs representing polyN repeat tracts of size 2-10 bp and dinucleotide repeat tracts of 1-5 repeated units, at genic regions.

In some embodiments, bootstrapping with replacement, randomly selecting the indels overlapping motifs at template and non-template strands from each randomly selected gene are performed for equal number of genes in multiple iterations, from which the standard deviation for the strand bias can be calculated.

The nullomers can be of any length. In some embodiments, the nullomers are in a length of from about 8 to about 50 nucleotides. In some embodiments, the nullomers are in a length of from about 10 to about 45 nucleotides. In some embodiments, the nullomers are in a length of from about 12 to about 40 nucleotides. In some embodiments, the nullomers are in a length of from about 14 to about 30 nucleotides. In some embodiments, the nullomers are in a length of from about 16 to about 20 nucleotides. In some embodiments, the nullomers are in a length of from about 8 nucleotides. In some embodiments, the nullomers are in a length of about 10 nucleotides. In some embodiments, the nullomers are in a length of about 11 nucleotides. In some embodiments, the nullomers are in a length of about 12 nucleotides. In some embodiments, the nullomers are in a length of about 13 nucleotides. In some embodiments, the nullomers are in a length of about 14 nucleotides. In some embodiments, the nullomers are in a length of about 15 nucleotides. In some embodiments, the nullomers are in a length of about 16 nucleotides. In some embodiments, the nullomers are in a length of about 17 nucleotides. In some embodiments, the nullomers are in a length of about 18 nucleotides. In some embodiments, the nullomers are in a length of about 19 nucleotides. In some embodiments, the nullomers are in a length of about 20 nucleotides. In some embodiments, the nullomers are in a length of about 25 nucleotides. In some embodiments, the nullomers are in a length of about 30 nucleotides. In some embodiments, the nullomers are in a length of about 35 nucleotides. In some embodiments, the nullomers are in a length of about 40 nucleotides. In some embodiments, the nullomers are in a length of about 45 nucleotides. In some embodiments, the nullomers are in a length of about 50 nucleotides. In some embodiments, the nullomers are in a length of more than about 50 nucleotides. Nullomers as Biomarkers for Cancer

The disclosure provides nullomers identified in cancers of numerous organs or tissues, including pancreas, esophagus, lymphoid, kidney, ovary, head and neck, lung, stomach, liver, CNS, uterus, skin, colorectal, prostate, bladder, bone and soft tissue, breast, biliary, cervix, thyroid and myeloid. The nullomers of the disclosure are provided in Table 1.

In some embodiments, the disclosure relates to a nullomer comprising at least about 60%, 65%, 70%, 75%, 80%, 85%, 86%, 87%, 88%, 89% 90%, 91%, 92%, 93%, 94%, 95%, 96%, 97%, 97%, 98, 99% or 100% sequence identity to any of the sequences provided in Table 1. In some embodiments, the disclosure relates to a nullomer comprising any of the sequences provided in Table 1. In some embodiments, the disclosure relates to a nucleic acid sequence that is complementary to any of the sequences provided in Table 1.

Lengthy table referenced here
US20240229157A1-20240711-T00001
Please refer to the end of the specification for access instructions.

### Biological Samples

The expression level of one or more disclosed nullomers can be determined in a biological sample obtained from a subject. A sample of a subject is one that originates from a subject. Such a sample may be further processed after it is obtained from the subject. For example, DNA or RNA may be isolated from a sample. In this example, the DNA or RNA isolated from the sample is also a sample obtained from the subject. A biological sample useful for determining the level of one or more disclosed nullomers may be obtained from essentially any source, including cells, blood, hair, tissues, and fluids throughout the body.

In some embodiments, the biological sample used for determining the level of one or more disclosed nullomers is a sample. In some embodiments the sample comprises circulating nullomers, e.g., extracellular nullomers. Extracellular nullomers freely circulate in a wide range of biological material, including bodily fluids, such as fluids from the circulatory system, e.g., a blood sample or a lymph sample, or from another bodily fluid such as urine or saliva or serum. Accordingly, in some embodiments, the biological sample used for determining the level of one or more disclosed nullomers is a bodily fluid, for example, blood, fractions thereof, serum, plasma, urine, saliva, tears, sweat, semen, vaginal secretions, lymph, bronchial secretions, CSF, whole blood, etc. In some embodiments, the sample is a sample that is obtained non-invasively. In some embodiments, the sample is whole blood or blood cells. In some embodiments, the sample is cells from a hair sample or nucleic acids from a hair sample. In some embodiments, the sample is sputum, saliva or spit. In some embodiments, the sample is a serum sample from a human. In some embodiments, the sample is a bodily fluid from a human. In some embodiments, the sample is a liquid biopsy from a human.

In some embodiments, any of the methods disclosed herein comprise using a small volume of sample for detection and/or diagnosis. In some embodiments, the sample used in any of the disclosed methods has a volume of no more than about 100 microliters of fluid. In some embodiments, the sample has a volume of no more than about 90 microliters of fluid. In some embodiments, the sample has a volume of no more than about 80 microliters of fluid. In some embodiments, the sample has a volume of no more than about 70 microliters of fluid. In some embodiments, the sample has a volume of no more than about 60 microliters of fluid. In some embodiments, the sample has a volume of no more than about 50 microliters of fluid. In some embodiments, the sample has a volume of no more than about 40 microliters of fluid. In some embodiments, the sample has a volume of no more than about 30 microliters of fluid. In some embodiments, the sample has a volume of no more than about 20 microliters of fluid. In some embodiments, the sample has a volume of no more than about 10 microliters of fluid. In some embodiments, the sample has a volume of no more than about 5 microliters of fluid. In some embodiments, the sample has a volume of no more than about 1 microliters of fluid.

In some embodiments, the disclosed methods comprise isolating total DNA or RNA and/or amplifying nullomers in a sample of no more than about 5 microliters, no more than about 10 microliters, no more than about 20 microliters, no more than about 40 microliters, no more than about 80 microliters, no more than about 100 microliters, no more than about 200 microliters, no more than about 300 microliters, no more than about 400 microliters, no more than about 500 microliters, no more than about 600 microliters, no more than about 700 microliters, no more than about 800 microliters, no more than about 900 microliters, no more than about 1 milliliter, no more than about 1.1 milliliters, no more than about 1.2 milliliters, no more than about 1.3 milliliters, no more than about 1.4 milliliters, no more than about 1.5 milliliters, no more than about 1.6 milliliters, no more than about 1.7 milliliters, no more than about 1.8 milliliters, no more than about 1.9 milliliters, or no more than about 2.0 milliliters. In some embodiments, the sample size is from about 1 microliters to about 2 milliliters, from about 20 microliters to about 2 milliliters, from about 5 microliters to about 1.5 milliliters, from about 10 microliters to about 500 microliters, from about 15 microliters to about 300 microliters, from about 20 microliters to about 200 microliters, from about 30 microliters to about 100 microliters, from about 1 microliters to about 100 microliters, from about 5 microliters to about 75 microliters, or from about 10 microliters to about 50 microliters of liquid sample in the form of subject plasma, whole blood, blood cells, cells from a hair sample, saliva or spit, or serum.

In some embodiments, the methods disclosed herein comprise isolating total DNA or RNA and/or amplifying nullomers in a sample of no more than about 5 microliters of serum, no more than about 10 microliters of serum, no more than about 20 microliters of serum, no more than about 40 microliters of serum, no more than about 80 microliters of serum, no more than about 100 microliters of serum, no more than about 200 microliters of serum, no more than about 300 microliters of serum, no more than about 400 microliters of serum, no more than about 500 microliters of serum, no more than about 600 microliters of serum, no more than about 700 microliters of serum, no more than about 800 microliters of serum, no more than about 900 microliters of serum, no more than about 1 milliliter of serum, no more than about 1.1 milliliters of serum, no more than about 1.2 milliliters of serum, no more than about 1.3 milliliters of serum, no more than about 1.4 milliliters of serum, no more than about 1.5 milliliters of serum, no more than about 1.6 milliliters of serum, no more than about 1.7 milliliters of serum, no more than about 1.8 milliliters of serum, no more than about 1.9 milliliters of serum, or no more than about 2.0 milliliters of serum.

Circulating nullomers include nullomers in cells, extracellular nullomers in microvesicles, in exosomes and extracellular nullomers that are not associated with cells or microvesicles (extracellular, non-vesicular nullomers). In some embodiments, the biological sample used for determining the level of one or more nullomers (e.g., a sample containing circulating nullomers) may contain cells. In other embodiments, the biological sample may be free or substantially free of cells (e.g., a serum sample). In some embodiments, a sample containing circulating nullomers, e.g., extracellular nullomers, is a blood-derived sample. Exemplary blood-derived sample types include, e.g., a plasma sample, a serum sample, a blood sample, etc. In other embodiments, a sample containing circulating nullomers is a lymph sample. Circulating nullomers are also found in urine and saliva, and biological samples derived from these sources are likewise suitable for determining the level of one or more disclosed nullomers.

In some embodiments, any of the methods of the disclosure comprises a step of isolating total DNA or RNA from a sample or cell or exosome or microvesicle. Methods of isolating DNA or RNA for expression analysis from blood, plasma and/or serum (see for example, Tsui NB et al. (2002) Clin. Chem. 48,1647-53, incorporated by reference in its entirety herein) and from urine (see for example, Boom R et al. (1990) J Clin Microbiol. 28, 495-503, incorporated by reference in its entirety herein) have been described and routinely used by the skilled person.

### Determining the Level of Nullomers in a Sample

The level of one or more disclosed nullomers in a biological sample can be determined by any suitable method. Any reliable method for measuring the level or amount of a nullomer in a sample can be used. Generally, nullomers can be detected and quantified from a sample (including fractions thereof), such as samples of isolated DNA or RNA by various methods known for DNA or mRNA, including, for example, amplification-based methods (e.g., Polymerase Chain Reaction (PCR), Real-Time Polymerase Chain Reaction (RT-PCR), Quantitative Polymerase Chain Reaction (qPCR), rolling circle amplification, etc.), hybridization-based methods (e.g., hybridization arrays (e.g., microarrays), NanoString analysis, Northern Blot analysis, branched DNA (bDNA) signal amplification, in situ hybridization, etc.), and sequencing-based methods (e.g., next-generation sequencing methods, for example, using the Illumina or IonTorrent platforms). Other exemplary techniques include ribonuclease protection assay (RPA) and mass spectroscopy.

In some embodiments where RNA is used as samples, RNA is converted to DNA (cDNA) prior to analysis. cDNA can be generated by reverse transcription of isolated RNA using conventional techniques. In some embodiments, nullomer is amplified prior to measurement. In other embodiments, the level of nullomer is measured during the amplification process. In still other embodiments, the level of nullomer is not amplified prior to measurement. Some exemplary methods suitable for determining the level of nullomer in a sample are described in greater detail below. These methods are provided by way of illustration only, and it will be apparent to a skilled person that other suitable methods may likewise be used.

### A. Amplification-Based Methods

Many amplification-based methods exist for detecting the level of nullomers, including, but not limited to, PCR, RT-PCR, qPCR, and rolling circle amplification. Other amplification-based techniques include, for example, ligase chain reaction, multiplex ligatable probe amplification, in vitro transcription (IVT), strand displacement amplification, transcription-mediated amplification, RNA (Eberwine) amplification, and other methods that are known to persons skilled in the art.

A typical PCR reaction includes multiple steps, or cycles, that selectively amplify target nucleic acid species: a denaturing step, in which a target nucleic acid is denatured; an annealing step, in which a set of PCR primers (i.e., forward and reverse primers) anneal to complementary DNA strands, and an elongation step, in which a thermostable DNA polymerase elongates the primers. By repeating these steps multiple times, a DNA fragment is amplified to produce an amplicon, corresponding to the target sequence. Typical PCR reactions include 20 or more cycles of denaturation, annealing, and elongation. In many cases, the annealing and elongation steps can be performed concurrently, in which case the cycle contains only two steps. A reverse transcription reaction (which produces a cDNA sequence having complementarity to a RNA) may be performed prior to PCR amplification. Reverse transcription reactions include the use of, e.g., a RNA-based DNA polymerase (reverse transcriptase) and a primer.

Kits for quantitative real time PCR of nullomers are known, and are commercially available. Examples of suitable kits include, but are not limited to, the TaqMan mRNA Assay (Applied Biosystems) and the mir Vana qRT-PCR nullomer detection kit (Ambion). The RNA can be ligated to a single stranded oligonucleotide containing universal primer sequences, a polyadenylated sequence, or adaptor sequence prior to reverse transcriptase and amplified using a primer complementary to the universal primer sequence, poly(T) primer, or primer comprising a sequence that is complementary to the adaptor sequence.

In some instances, custom qRT-PCR assays can be developed for determination of nullomer levels. Custom qRT-PCR assays to measure nullomers in a biological sample, e.g., a body fluid, can be developed using, for example, methods that involve an extended reverse transcription primer and locked nucleic acid modified PCR. Custom nullomer assays can be tested by running the assay on a dilution series of chemically synthesized nullomer corresponding to the target sequence. This permits determination of the limit of detection and linear range of quantitation of each assay. Furthermore, when used as a standard curve, these data permit an estimate of the absolute abundance of nullomers measured in biological samples.

Amplification curves may optionally be checked to verify that Ct values are assessed in the linear range of each amplification plot. Typically, the linear range spans several orders of magnitude. For each candidate nullomer assayed, a chemically synthesized version of the nullomer can be obtained and analyzed in a dilution series to determine the limit of sensitivity of the assay, and the linear range of quantitation. Relative expression levels may be determined, for example, as described by Livak et al., Methods (2001) December; 25(4):402-8.

In some embodiments, two or more nullomers are amplified in a single reaction volume. For example, multiplex q-PCR, such as qRT-PCR, enables simultaneous amplification and quantification of at least two nullomers of interest in one reaction volume by using more than one pair of primers and/or more than one probe. The primer pairs comprise at least one amplification primer that specifically binds each nullomer, and the probes are labeled such that they are distinguishable from one another, thus allowing simultaneous quantification of multiple nullomers.

Rolling circle amplification is a DNA-polymerase driven reaction that can replicate circularized oligonucleotide probes with either linear or geometric kinetics under isothermal conditions (see, for example, Lizardi et al., Nat. Gen. (1998) 19(3):225-232; Gusev et al., Am. J. Pathol. (2001) 159(1):63-69; Nallur et al., Nucleic Acids Res. (2001) 29(23):E118). In the presence of two primers, one hybridizing to the (+) strand of DNA, and the other hybridizing to the (−) strand, a complex pattern of strand displacement results in the generation of over 109 copies of each DNA molecule in 90 minutes or less. Tandemly linked copies of a closed circle DNA molecule may be formed by using a single primer. The process can also be performed using a matrix-associated DNA. The template used for rolling circle amplification may be reverse transcribed. This method can be used as a highly sensitive indicator of nullomer sequence and expression level at very low nullomer concentrations (see, for example, Cheng et al., Angew Chem. Int. Ed. Engl. (2009) 48(18):3268-72; Neubacher et al., Chembiochem. (2009) 10(8):1289-91).

In some embodiments, the disclosure provide a method for identifying the presence, absence, or quantity of one or a plurality of the disclosed nullomers comprising: a) isolating nucleic acids from a sample; and b) mixing the nucleic acids with one or a plurality of primers under conditions and for a period of time sufficient to allow amplification of the one or plurality nullomers, wherein the one or plurality of primers comprises sequences that are complementary to any of the nullomers provided in Table 1. In some embodiments, the nucleic acid from a sample is cell-free (cfDNA). In some embodiments, the nucleic acid from a sample is circulating tumor (ctDNA). In some embodiments, the primer used in the disclosed method comprises from about 6 to about 16 nucleotides. In some embodiments, the primer used in the disclosed method comprises from about 7 to about 15 nucleotides. In some embodiments, the primer used in the disclosed method comprises from about 8 to about 14 nucleotides. In some embodiments, the primer used in the disclosed method comprises about 6 nucleotides. In some embodiments, the primer used in the disclosed method comprises about 7 nucleotides. In some embodiments, the primer used in the disclosed method comprises about 8 nucleotides. In some embodiments, the primer used in the disclosed method comprises about 9 nucleotides. In some embodiments, the primer used in the disclosed method comprises about 10 nucleotides. In some embodiments, the primer used in the disclosed method comprises about 11 nucleotides. In some embodiments, the primer used in the disclosed method comprises about 12 nucleotides. In some embodiments, the primer used in the disclosed method comprises about 13 nucleotides. In some embodiments, the primer used in the disclosed method comprises about 14 nucleotides. In some embodiments, the primer used in the disclosed method comprises about 15 nucleotides. In some embodiments, the primer used in the disclosed method comprises about 16 nucleotides.

In some embodiments, the identification of the presence or quantity of one or a plurality of the disclosed nullomers is indicative that the subject from which the sample is obtained has the cancer type corresponding to the particular nullomer identified in Table 1.

### B. Hybridization-Based Methods

Nullomers may be detected using hybridization-based methods, including but not limited to hybridization arrays (e.g., microarrays), NanoString analysis, Southern Blot analysis, Northern Blot analysis, branched DNA (bDNA) signal amplification, and in situ hybridization.

Microarrays can be used to measure the levels of large numbers of nullomers simultaneously. Microarrays can be fabricated using a variety of technologies, including printing with fine-pointed pins onto glass slides, photolithography using pre-made masks, photolithography using dynamic micromirror devices, ink-jet printing, or electrochemistry on microelectrode arrays. Also useful are microfluidic TaqMan Low-Density Arrays, which are based on an array of microfluidic qRT-PCR reactions, as well as related microfluidic qRT-PCR based methods.

Axon B-4000 scanner and Gene-Pix Pro 4.0 software or other suitable software can be used to scan images. Non-positive spots after background subtraction, and outliers detected by the ESD procedure, are removed. The resulting signal intensity values are normalized to per-chip median values and then used to obtain geometric means and standard errors for each nullomer. Each signal can be transformed to log base 2, and a one-sample t test can be conducted. Independent hybridizations for each sample can be performed on chips with each nullomer spotted multiple times to increase the robustness of the data.

Microarrays can be used for the expression profiling of nullomers in diseases. For example, DNA or RNA can be extracted from a sample and, optionally, the nullomers are size-selected from total DNA or RNA. Oligonucleotide linkers can be attached to the 5′ and 3′ ends of the nullomers and the resulting ligation products are used as templates for an RT-PCR reaction. The sense strand PCR primer can have a fluorophore attached to its 5′ end, thereby labeling the sense strand of the PCR product. The PCR product is denatured and then hybridized to the microarray. A PCR product, referred to as the target nucleic acid that is complementary to the corresponding nullomer capture probe sequence on the array will hybridize, via base pairing, to the spot at which the, capture probes are affixed. The spot will then fluoresce when excited using a microarray laser scanner. In some embodiments, probes of the disclosure are nucleic acid sequences comprising from about 10 to about 20 nucleotides in length and are DNA or RNA or NDA/RNA hybrid seqeunces complementary to a nullomer of Table 1, Table 4, Table 5 or Table 7. In some embodiments, the disclosure relate to composition comprising one or a plurality f such probes. And in some embodiments, those probes comprise a fluorescent probe detectable when exposed to light emitted onto the probe.

The fluorescence intensity of each spot is then evaluated in terms of the number of copies of a particular nullomer, using a number of positive and negative controls and array data normalization methods, which will result in assessment of the level of expression of a particular nullomer.

Total RNA containing the nullomers extracted from a body fluid sample can also be used directly without size-selection of the nullomers. For example, the RNA can be 3′ end labeled using T4 RNA ligase and a fluorophore-labeled short RNA linker. Fluorophore-labeled nullomers complementary to the corresponding nullomer capture probe sequences on the array hybridize, via base pairing, to the spot at which the capture probes are affixed. The fluorescence intensity of each spot is then evaluated in terms of the number of copies of a particular nullomer, using a number of positive and negative controls and array data normalization methods, which will result in assessment of the level of expression of a particular nullomer.

Several types of microarrays can be employed including, but not limited to, spotted oligonucleotide microarrays, pre-fabricated oligonucleotide microarrays or spotted long oligonucleotide arrays.

Nullomers can also be detected without amplification using the nCounter Analysis System (NanoString Technologies, Seattle, Wash.). This technology employs two nucleic acid-based probes that hybridize in solution (e.g., a reporter probe and a capture probe). After hybridization to a nullomers disclosed herein, excess probes are removed, and probe/target complexes are analyzed in accordance with the manufacturer's protocol. nCounter nullomer assay kits are available from NanoString Technologies, which are capable of distinguishing between highly similar nullomers with great specificity.

Nullomers can also be detected using branched DNA (bDNA) signal amplification (see, for example, Urdea, Nature Biotechnology (1994), 12:926-928). RNA assays based on bDNA signal amplification are commercially available. One such assay is the QuantiGene.RTM. 2.0 nullomer Assay (Affymetrix, Santa Clara, Calif.). Southern Blot, Northern Blot and in situ hybridization may also be used to detect nullomers. Suitable methods for performing Southern Blot, Northern Blot and in situ hybridization are known in the art.

In some embodiments, biomarker expression is determined by an assay known to those of skill in the art, including but not limited to, multi-analyte profile test, enzyme-linked immunosorbent assay (ELISA), radioimmunoassay, Western blot assay, immunofluorescent assay, enzyme immunoassay, immunoprecipitation assay, chemiluminescent assay, immunohistochemical assay, dot blot assay, or slot blot assay. In some embodiments, wherein an antibody is used in the assay the antibody is detectably labeled. The antibody labels may include, but are not limited to, immunofluorescent label, chemiluminescent label, phosphorescent label, enzyme label, radiolabel, avidin/biotin, colloidal gold particles, colored particles, and magnetic particles. In some embodiments, biomarker expression is determined by an IHC assay.

In some embodiments, biomarker expression is determined using an agent that specifically binds the biomarker. Any molecular entity that displays specific binding to a biomarker can be employed to determine the level of that biomarker protein in a sample. Specific binding agents include, but are not limited to, antibodies, antibody fragments, antibody mimetics, and polynucleotides (e.g., aptamers). One of skill understands that the degree of specificity required is determined by the particular assay used to detect the biomarker protein. In some embodiments, the disclosure relates to a system comprising a solid support (such as an ELISA plate, gel, bead or column comprising an antibody, antibody fragment, antibody mimetic, and/or polynucleotides capable of binding to T3p or a salt thereof.

### C. Sequencing-Based Methods

Advanced sequencing methods can likewise be used as available. For example, nullomers can be detected using Illumina. Next Generation Sequencing (e.g., Sequencing-By-Synthesis or TruSeq methods, using, for example, the HiSeq, HiScan, GenomeAnalyzer, or MiSeq systems (Illumina, Inc., San Diego, Calif.)). Nullomers can also be detected using Ion Torrent Sequencing (Ion Torrent Systems, Inc., Gulliford, Conn.), or other suitable methods of semiconductor sequencing.

### D. Additional Nullomers Detection Tools

Mass spectroscopy can be used to quantify nullomers using RNase mapping. Isolated RNAs can be enzymatically digested with RNA endonucleases (RNases) having high specificity (e.g., RNase TI, which cleaves at the 3′-side of all unmodified guanosine residues) prior to their analysis by MS or tandem MS (MS/MS) approaches. The first approach developed utilized the on-line chromatographic separation of endonuclease digests by reversed phase HPLC coupled directly to ESI-MS. The presence of posttranscriptional modifications can be revealed by mass shifts from those expected based upon the RNA sequence. Ions of anomalous mass/charge values can then be isolated for tandem MS sequencing to locate the sequence placement of the posttranscriptionally modified nucleoside.

Matrix-assisted laser desorption/ionization mass spectrometry (MALDI-MS) has also been used as an analytical approach for obtaining information about posttranscriptionally modified nucleosides. MALDI-based approaches can be differentiated from ESI-based approaches by the separation step. In MALDI-MS, the mass spectrometer is used to separate the nullomers.

To analyze a limited quantity of intact nullomers, a system of capillary LC coupled with nanoESI-MS can be employed, by using a linear ion trap-orbitrap hybrid mass spectrometer (LTQ Orbitrap XL, Thermo Fisher Scientific) or a tandem-quadrupole time-of-flight mass spectrometer (QSTAR XL, Applied Biosystems) equipped with a custom-made nanospray ion source, a Nanovolume Valve (Valco Instruments), and a splitless nano HPLC system (DiNa, KYA Technologies). Analyte/TEAA is loaded onto a nano-LC trap column, desalted, and then concentrated. Intact nullomers are eluted from the trap column and directly injected into a Cl 8 capillary column, and chromatographed by RP-HPLC using a gradient of solvents of increasing polarity. The chromatographic eluent is sprayed from a sprayer tip attached to the capillary column, using an ionization voltage that allows ions to be scanned in the negative polarity mode.

Additional methods for nullomer detection and measurement include, for example, strand invasion assay (Third Wave Technologies, Inc.), surface plasmon resonance (SPR), cDNA, MTDNA (metallic DNA; Advance Technologies, Saskatoon, SK), and single-molecule methods such as the one developed by US Genomics. Multiple nullomers can be detected in a microarray format using a novel approach that combines a surface enzyme reaction with nanoparticle-amplified SPR imaging (SPRI). The surface reaction of poly(A) polymerase creates poly(A) tails on nullomers hybridized onto locked nucleic acid (LNA) microarrays. DNA-modified nanoparticles are then adsorbed onto the poly(A) tails and detected with SPRI. This ultrasensitive nanoparticle-amplified SPRI methodology can be used for nullomers profiling at attomole levels. IN some embodiments, CRISPR-Cas9 complexes can be used to detect the presence of nullomers in vitro based upon exposure of a sample from a patient to sgRNA-Cas protein complex, wherein the sgRNA is complementary to at least a portion of the nullomer sequence. In some embodiments, the exposure is to genomic DNA within a cancer cell.

In some embodiments, the disclosure relates to a composition or system comprising one or a plurality of sgRNAs that comprise about 70%, 75%, 80%, 85%, 90%, 91%, 92%, 93%, 94%, 95%, 96%, 97%, 98%, or 99% to the sequences of Table 6. In some embodiments, the disclosure relates to a composition or system comprising one or a plurality of sgRNAs that comprise from about 98 to about 110 nucleotides in length with at least one portion of the sgRNA complementary to a nucleic sequence from about 8 to about 18 nuceotides of any nullomer disclosed in Table 1, Table 4, Table 5 or Table 7.

As used herein, the term “mutagen” means any molecule, a nucleic acid sequence, amino acid sequence, or hybrid amino acid or nucleic acid sequence that causes a mutation or modification in one or more regions of endogenous nucleic acid when exposed for a time period sufficient to cause the mutation. In some embodiments, the mutation is a point mutation, frameshift mutation, deletion, truncation, or addition. In some embodiments, the mutagen is a vector or a gene-modifying enzyme.

The term “vector” as used herein refers to any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors.

The term “gene-modifying enzyme” as used herein refers to an enzyme that is capable of modifying a gene by introducing a mutation (e.g., point mutation, frameshift mutation, deletion, or truncation) causing gene inactivation or introducing heterologous nucleotides (e.g., genes) through non-homologous end joining or homologous recombination. Exemplary gene-modifying enzymes, include but not limited to, a Cas protein, a meganuclease, a transcription activator-like effector nucleases (TALEN), a transposon, a zinc-finger nuclease (ZFN), or a recombinase. In some embodiments, the gene-modifying enzyme suitable for the methods disclosed herein is a Cas protein, a meganuclease, a TALEN, a ZFN, or a recombinase. In some embodiments, the gene-modifying enzyme suitable for the methods disclosed herein is a Cas protein. In some preferred embodiments, the gene-modifying enzyme suitable for the methods disclosed herein is a Cas9 protein.

The term “Cas9 protein” refers to the “clustered, regularly interspaced, short palindromic repeats (CRISPR)-associated protein 9.” This term is well known in the art and has been described, e.g. in Makarova et al. (2011) Nat. Rev. Microbiol., 9:467-477, and in Makarova et al. (2011) Biol. Direct., 6:38. Cas proteins are endonuclease that form part of an adaptive defense mechanism evolved by bacteria and archaea to protect them from invading viruses and plasmids. Cas9 protein or gene information can be obtained from a known database such as the GenBank of NCBI (National Center for Biotechnology Information), but is not limited thereto. Moreover, the Cas9 protein may comprise not only wild-type Cas9, but also deactivated Cas9 (dCas9), or Cas9 variants such as Cas9 nickase. The deactivated Cas9 may be RFN (RNA-guided FokI nuclease) comprising a FokI nuclease domain bound to dCas9, or may be dCas9 to which a transcription activator or repressor domain is bound. In addition, the Cas9 protein is not limited in its origin. For example, the Cas9 protein may be derived from Streptococcus pyogenes, Francisella novicida, Streptococcus thermophilus, Legionella pneumophila, Listeria innocua, or Streptococcus mutans.

Cas9 protein is the major protein element of the CRISPR/Cas9 system, which forms a complex with crRNA (CRISPR RNA) and tracrRNA (trans-activating crRNA) to form activated endonuclease or nickase. “CRISPR system” refers collectively to transcripts or synthetically produced transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a nucleic acid sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides, but in some embodiments, the tragte sequence is a nullomer or a region of a nullomer that is from about 10 to about 35 nucleotides of the nullomer sequence of any nullomer from Table 1. In some embodiments, the target sequence is a DNA polynucleotide and is referred to a DNA target sequence. In some embodiments, a target sequence comprises at least three nucleic acid sequences that are recognized by a Cas-protein when the Cas protein is associated with a CRISPR complex or system which comprises at least one sgRNA or one tracrRNA/crRNA duplex at a concentration and within an microenvironment suitable for association of such a system. In some embodiments, the target DNA comprises at least one or more proto-spacer adjacent motifs which sequences are known in the art and are dependent upon the Cas protein system being used in conjunction with the sgRNA or crRNA/tracrRNAs employed by this work. In some embodiments, the target DNA comprises NNG, where G is a guanine and N is any naturally occurring nucleic acid. In some embodiments the target DNA comprises any one or combination of NNG, NNA, GAA, NNAGAAW and NGGNG, where G is an guanine, A is adenine, and Nis any naturally occurring nucleic acid from one nullomer in Table 1.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. As with the target sequence, it is believed that complete complementarity is not needed, provided there is sufficient to be functional (bind the Cas protein or functional fragment thereof). In some embodiments, the tracr sequence has at least 50%, 60%, 70%, 80%, 90%, 95% or 99% of sequence complementarity along the length of the tracr mate sequence when optimally aligned. In some embodiments, one or more vectors driving expression of one or more elements of a CRISPR system are introduced into a host cell such that the presence and/or expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. In some embodiments, the target site is a genomic DNA of a cancer cell within the host or a cancer cell isolated from the subject in a sample or within a system independent of a tumor.

With at least some of the modification contemplated by this disclosure, in some embodiments, the guide sequence or RNA or DNA sequences that form a CRISPR complex are at least partially synthetic. The CRISPR system elements that are combined in a single vector may be arranged in any suitable orientation, such as one element located 5′ with respect to (“upstream” of) or 3′ with respect to (“downstream” of) a second element. In some embodiments, the disclosure relates to a composition comprising a chemically synthesized guide sequence. In some embodiments, the chemically synthesized guide sequence is used in conjunction with a vector comprising a coding sequence that encodes a CRISPR enzyme, such as a type II Cas9 protein. In some embodiments, the chemically synthesized guide sequence is used in conjunction with one or more vectors, wherein each vector comprises a coding sequence that encodes a CRISPR enzyme, such as a type II Cas9 protein. The coding sequence of one element may be located on the same or opposite strand of the coding sequence of a second element, and oriented in the same or opposite direction. In some embodiments, a single promoter drives expression of a transcript encoding a CRISPR enzyme and one or more additional (second, third, fourth, etc.) guide sequences, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, one or more additional guide sequence, tracr mate sequence, and tracr sequence are each a component of different nucleic acid sequences. For instance, in the case of a tracr and tracr mate sequences and in some embodiments, the disclosure relates to a composition comprising at least a first and second nucleic acid sequence, wherein the first nucleic acid sequence comprises a tracr sequence and the second nucleic acid sequence comprises a tracr mate sequence, wherein the first nucleic acid sequence is at least partially complementary to the second nucleic acid sequence such that the first and second nucleic acid for a duplex and wherein the first nucleic acid and the second nucleic acid either individually or collectively comprise a DNA-targeting domain, a Cas protein binding domain, and a transcription terminator domain. In some embodiments, the CRISPR enzyme, one or more additional guide sequence, tracr mate sequence, and tracr sequence are operably linked to and expressed from the same promoter. In some embodiments, the disclosure relates to compositions comprising any one or combination of the disclosed domains on one guide sequence or two separate tracrRNA/crRNA sequences with or without any of the disclosed modifications. Any methods disclosed herein also relate to the use of tracrRNA/crRNA sequence interchangeably with the use of a guide sequence, such that a composition may comprise a single synthetic guide sequence and/or a synthetic tracrRNA/crRNA with any one or combination of modified domains disclosed herein.

The CRISPR system suitable for the present disclosure can also comprise a modified CRISPR enzyme (or “Cas protein”) or a nucleotide sequence encoding one or more Cas proteins. Any protein capable of enzymatic activity in cooperation with a guide sequence is a Cas protein. In some embodiments, the disclosure relates to a system comprises a vector comprising a regulatory element operably linked to an enzyme-coding sequence encoding a CRISPR enzyme, such as a Cas protein from the Cas family of enzymes. In some embodiments, the disclosure relates to a system, composition, or pharmaceutical composition comprising any one or plurality of Cas proteins either individually or in combination with one or a plurality of guide sequences. Compositions of one or a plurality of Cas proteins may be administered to a subject with any of the disclosed guide sequences sequentially or contemporaneously. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, type V CRISPR-Cas systems (e.g., Cas12), and Type VI CRISPR-Cas systems (e.g., Cas13), and variants and fragments thereof, or modified versions thereof having at least 70% sequence identity to any of the above Cas proteins. These enzymes are known, for example, the amino acid sequence of S. pyogenes Cas9 protein may be found in the SwissProt database under accession number Q99ZW2. In some embodiments, the unmodified CRISPR enzyme has DNA cleavage activity, such as Cas9. In some embodiments the CRISPR enzyme is Cas9, and may be Cas9 from S. pyogenes or S. pneumoniae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme or Cas protein that is mutated to with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. In some embodiments, a Cas9 nickase may be used in combination with guide sequence(s), e.g., two guide sequences, which target respectively sense and antisense strands of the DNA target. This combination allows both strands to be nicked and used to induce NHEJ.

As a further example, two or more catalytic domains of Cas9 (RuvC I, RuvC II, and RuvC III) may be mutated to produce a mutated Cas9 substantially lacking all DNA cleavage activity. In some embodiments, a D10A mutation is combined with one or more of H840A, N854A, or N863A mutations to produce a Cas9 enzyme substantially lacking all DNA cleavage activity. In some embodiments, a CRISPR enzyme is considered to substantially lack all DNA cleavage activity when the DNA cleavage activity of the mutated enzyme is less than about 25%, 10%, 5%, 1%, 0.1%, 0.01%, or lower with respect to its non-mutated form. Other mutations may be useful; where the Cas9 or other CRISPR enzyme is from a species other than S. pyogenes, mutations in corresponding amino acids may be made to achieve similar effects.

The disclosure relates to a method of detecting the presence of a nullomer by exposing a Cas protein and sgRNA specific to a target nullomer sequence to a nullomer target sequence. In some embodiments, the nullomer target sequence is any nullomer from Table 1 and the sgRNA sequence specific for the nullomer is any RNA molecule that comprises from about 10 to about 35 nucleotides complementary to a nullomer in Table 1. In some embodiments, the method further comprises allowing a time period sufficient for the sgRNA to associate with the nullomer and the Cas protein to excise the nullomer from the genomic DNA of a host cell or cell within a sample. Detection of the nullomer can further comprise identifying the nullomer sequence excised from the cell by amplification through PCR or a non-amplification event such as those disclosed herein.

In certain embodiments, labels, dyes, or labeled probes and/or primers are used to detect amplified or unamplified nullomers. The skilled artisan will recognize which detection methods are appropriate based on the sensitivity of the detection method and the abundance of the target. Depending on the sensitivity of the detection method and the abundance of the target, amplification may or may not be required prior to detection. One skilled in the art will recognize the detection methods where nullomer amplification is preferred.

A probe or primer may include standard (A, T or U, G and C) bases, or modified bases. Modified bases include, but are not limited to, the AEGIS bases (from Eragen Biosciences), which have been described, e.g., in U.S. Pat. Nos. 5,432,272, 5,965,364, and 6,001,983. In certain aspects, bases are joined by a natural phosphodiester bond or a different chemical linkage. Different chemical linkages include, but are not limited to, a peptide bond or a Locked Nucleic Acid (LNA) linkage, which is described, e.g., in U.S. Pat. No. 7,060,809.

In a further aspect, oligonucleotide probes or primers present in an amplification reaction are suitable for monitoring the amount of amplification product produced as a function of time. In certain aspects, probes having different single stranded versus double stranded character are used to detect the nucleic acid. Probes include, but are not limited to, the 5′-exonuclease assay (e.g., TAQMAN) probes (see U.S. Pat. No. 5,538,848), stem-loop molecular beacons (see, e.g., U.S. Pat. Nos. 6,103,476 and 5,925,517), stemless or linear beacons (see, e.g., WO 9921881, U.S. Pat. Nos. 6,485,901 and 6,649,349), peptide nucleic acid (PNA) Molecular Beacons (see, e.g., U.S. Pat. Nos. 6,355,421 and 6,593,091), linear PNA beacons (see, e.g. U.S. Pat. No. 6,329,144), non-FRET probes (see, e.g., U.S. Pat. No. 6,150,097), Sunrise.TM./AmplifluorB.TM. probes (see, e.g., U.S. Pat. No. 6,548,250), stem-loop and duplex SCORPION probes (see, e.g., U.S. Pat. No. 6,589,743), bulge loop probes (see, e.g., U.S. Pat. No. 6,590,091), pseudo knot probes (see, e.g., U.S. Pat. No. 6,548,250), cyclicons (see, e.g., U.S. Pat. No. 6,383,752), MGB Eclipse™ probe (Epoch Biosciences), hairpin probes (see, e.g., U.S. Pat. No. 6,596,490), PNA light-up probes, antiprimer quench probes (Li et al., Clin. Chem. 53:624-633 (2006)), self-assembled nanoparticle probes, and ferrocene-modified probes described, for example, in U.S. Pat. No. 6,485,901.

In certain embodiments, one or more of the primers in an amplification reaction can include a label. In yet further embodiments, different probes or primers comprise detectable labels that are distinguishable from one another. In some embodiments, a nucleic acid, such as the probe or primer, may be labeled with two or more distinguishable labels.

In some aspects, a label is attached to one or more probes and has one or more of the following properties: (i) provides a detectable signal; (ii) interacts with a second label to modify the detectable signal provided by the second label, e.g., FRET (Fluorescent Resonance Energy Transfer); (iii) stabilizes hybridization, e.g., duplex formation; and (iv) provides a member of a binding complex or affinity set, e.g., affinity, antibody-antigen, ionic complexes, hapten-ligand (e.g., biotin-avidin). In still other aspects, use of labels can be accomplished using any one of a large number of known techniques employing known labels, linkages, linking groups, reagents, reaction conditions, and analysis and purification methods.

Nullomers can be detected by direct or indirect methods. In a direct detection method, one or more nullomers are detected by a detectable label that is linked to a nucleic acid molecule. In such methods, the nullomers may be labeled prior to binding to the probe. Therefore, binding is detected by screening for the labeled nullomer that is bound to the probe. The probe is optionally linked to a bead in the reaction volume.

In certain embodiments, nucleic acids are detected by direct binding with a labeled probe, and the probe is subsequently detected. In some embodiments, the nucleic acids, such as amplified nullomers, are detected using FlexMAP Microspheres (Luminex) conjugated with probes to capture the desired nucleic acids. Some methods may involve detection with polynucleotide probes modified with fluorescent labels or branched DNA (bDNA) detection, for example.

In some embodiments, biomarker expression is determined using a PCR-based assay comprising specific primers and/or probes for each biomarker. As used herein, the term “probe” refers to any molecule that is capable of selectively binding a specifically intended target biomolecule. In some embodiments, as used herein, the term “probe” refers to any molecule that may bind or associate, indirectly or directly, covalently or non-covalently, to any of the substrates and/or reaction products and/or proteases disclosed herein and whose association or binding is detectable using the methods disclosed herein. In some embodiments, the term “probe” refers to any molecule comprising a nucleic acid sequence that is complementary to any of the nucleic acid sequences disclosed in TABLE 1 or one comprising at least about 70%, 80%, 81%, 82%, 83%, 84, 85%, 86%, 87%, 88%, 89%, 90%, 91%, 92%, 93%, 94%, 95%, 96%, 97%, 98% or 99% sequence identity to any of the nucleic acid sequences disclosed in TABLE 1. In some embodiments, the term “probe” refers to any molecule comprising a nucleic acid sequence that is complementary to a fragment of any of the nucleic acid sequences disclosed in TABLE 1 or one comprising at least about 70%, 80%, 81%, 82%, 83%, 84, 85%, 86%, 87%, 88%, 89%, 90%, 91%, 92%, 93%, 94%, 95%, 96%, 97%, 98% or 99% sequence identity to a fragment of any of the nucleic acid sequences disclosed in TABLE 1. In some embodiments, the term “probe” refers to a sgRNA molecule comprising a nucleic acid sequence that is complementary to a fragment of any of the nucleic acid sequences disclosed in TABLE 1 or one comprising at least about 70%, 80%, 81%, 82%, 83%, 84, 85%, 86%, 87%, 88%, 89%, 90%, 91%, 92%, 93%, 94%, 95%, 96%, 97%, 98% or 99% sequence identity to a fragment of any of the nucleic acid sequences disclosed in TABLE 1. In some embodiments, the probe is a fluorogenic probe, antibody or absorbance-based probes. If an absorbance-based probe, the chromophore pNA (para-nitroanaline) may be used as a probe for detection and/or quantification of a target nucleic acid sequence disclosed herein. In some embodiments, the probe may comprise a nucleic acid sequence labeled with a fluorogenic molecule or a substrate that when exposed to an enzyme becomes fluorogenic and the nucleic acid sequence is complementary to any of the nucleic acid sequences disclosed in TABLE 1 or one comprising at least about 70%, 80%, 81%, 82%, 83%, 84, 85%, 86%, 87%, 88%, 89%, 90%, 91%, 92%, 93%, 94%, 95%, 96%, 97%, 98% or 99% sequence identity to any of the nucleic acid sequences disclosed in TABLE 1. Probes can be synthesized by one of skill in the art using known techniques, or derived from biological preparations. Probes may include but are not limited to, RNA, DNA, proteins, peptides, aptamers, antibodies, and organic molecules. The term “primer” or “probe” encompasses oligonucleotides that have a specific sequence or oligoribonucleotides that have a specific sequence. In some embodiments, the probe are from about 5 to about 20 nucleotides in length and are complementary to the nucleic acid sequences in TABLE 1 and comprise at least about 70%, 80%, 81%, 82%, 83%, 84, 85%, 86%, 87%, 88%, 89%, 90%, 91%, 92%, 93%, 94%, 95%, 96%, 97%, 98%, 99% or about 100% sequence identity to any one or combination of nucleic acid sequences complementary to those provided in TABLE 1. In some embodiments, the probe are from about 5 to about 20 nucleotides in length and are complementary to the nucleic acid sequences in TABLE 1 and comprise at least about 70%, 80%, 81%, 82%, 83%, 84, 85%, 86%, 87%, 88%, 89%, 90%, 91%, 92%, 93%, 94%, 95%, 96%, 97%, 98%, 99% or about 100% sequence identity to any one or combination of nucleic acid sequences complementary to those provided in TABLE 7. In some embodiments, the probe are from about 5 to about 20 nucleotides in length and are complementary to the nucleic acid sequences in TABLE 1 and comprise at least about 70%, 80%, 81%, 82%, 83%, 84, 85%, 86%, 87%, 88%, 89%, 90%, 91%, 92%, 93%, 94%, 95%, 96%, 97%, 98%, 99% or about 100% sequence identity to any one or combination of nucleic acid sequences complementary to those provided in TABLE 8.

The target molecule could be any one or a combination of nucleic acid sequences identified in TABLE 1. In some embodiments, the target molecule is a nucleic acid sequence comprising at least about 70%, 80%, 81%, 82%, 83%, 84, 85%, 86%, 87%, 88%, 89%, 90%, 91%, 92%, 93%, 94%, 95%, 96%, 97%, 98%, or about 99% sequence identity to any one or combination of nucleic acid sequences provided in TABLE 1. In some embodiments, the target molecule is any amplified fragment of any one or combination of nucleic acid sequences identified in TABLE 1, and/or any one or combination of nucleic acid sequence comprising at least about 70%, 80%, 81%, 82%, 83%, 84, 85%, 86%, 87%, 88%, 89%, 90%, 91%, 92%, 93%, 94%, 95%, 96%, 97%, 98%, or about 99% sequence identity to any one or combination of nucleic acid sequences in TABLE 1.

In other embodiments, nucleic acids are detected by indirect detection methods. For example, a biotinylated probe may be combined with a streptavidin-conjugated dye to detect the bound nucleic acid. The streptavidin molecule binds a biotin label on amplified nullomer, and the bound nullomer is detected by detecting the dye molecule attached to the streptavidin molecule. In some embodiments, the streptavidin-conjugated dye molecule comprises PHYCOLINK. Streptavidin R-Phycoerythrin (PROzyme). Other conjugated dye molecules are known to persons skilled in the art.

Labels include, but are not limited to, light-emitting, light-scattering, and light-absorbing compounds which generate or quench a detectable fluorescent, chemiluminescent, or bioluminescent signal (see, e.g., Kricka, L., Nonisotopic DNA Probe Techniques, Academic Press, San Diego (1992) and Garman A., Non-Radioactive Labeling, Academic Press (1997)). A dual labeled fluorescent probe that includes a reporter fluorophore and a quencher fluorophore is used in some embodiments. It will be appreciated that pairs of fluorophores are chosen that have distinct emission spectra so that they can be easily distinguished.

In certain embodiments, labels are hybridization-stabilizing moieties which serve to enhance, stabilize, or influence hybridization of duplexes, e.g., intercalators and intercalating dyes (including, but not limited to, ethidium bromide and SYBR-Green), minor-groove binders, and cross-linking functional groups (see, e.g., Blackburn et al., eds. “DNA and RNA Structure” in Nucleic Acids in Chemistry and Biology (1996)).

In other embodiments, methods relying on hybridization and/or ligation to quantify nullomers may be used, including oligonucleotide ligation (OLA) methods and methods that allow a distinguishable probe that hybridizes to the target nucleic acid sequence to be separated from an unbound probe. As an example, HARP-like probes, as disclosed in U.S. Publication No. 2006/0078894 may be used to measure the quantity of nullomers. In such methods, after hybridization between a probe and the targeted nucleic acid, the probe is modified to distinguish the hybridized probe from the unhybridized probe. Thereafter, the probe may be amplified and/or detected. In general, a probe inactivation region comprises a subset of nucleotides within the target hybridization region of the probe. To reduce or prevent amplification or detection of a HARP probe that is not hybridized to its target nucleic acid, and thus allow detection of the target nucleic acid, a post-hybridization probe inactivation step is carried out using an agent which is able to distinguish between a HARP probe that is hybridized to its targeted nucleic acid sequence and the corresponding unhybridized HARP probe. The agent is able to inactivate or modify the unhybridized HARP probe such that it cannot be amplified. A probe ligation reaction may also be used to quantify nullomers. In a Multiplex Ligation-dependent Probe Amplification (MLPA) technique (Schouten et al., Nucleic Acids Research 30:e57 (2002)), pairs of probes which hybridize immediately adjacent to each other on the target nucleic acid are ligated to each other driven by the presence of the target nucleic acid. In some aspects, MLPA probes have flanking PCR primer binding sites. MLPA probes are specifically amplified when ligated, thus allowing for detection and quantification of nullomer biomarkers.

### Detecting a Level of Nullomers

The nullomers described herein can be used individually or in combination in diagnostic tests to assess the type of cancer, tissue of origin, and status or stage of the cancer in a subject. Cancer status or stage includes the presence or absence of the cancer. Cancer status or stage may also include monitoring the course of the cancer, for example, monitoring disease progression. Based on the cancer status or stage of a subject, additional procedures may be indicated, including, for example, additional diagnostic tests or therapeutic procedures.

The power of a diagnostic test to correctly predict disease status is commonly measured in terms of the accuracy of the assay, the sensitivity of the assay, the specificity of the assay, or the “Area Under a Curve” (AUC), for example, the area under a Receiver Operating Characteristic (ROC) curve. As used herein, accuracy is a measure of the fraction of misclassified samples. Accuracy may be calculated as the total number of correctly classified samples divided by the total number of samples, e.g., in a test population. Sensitivity is a measure of the “true positives” that are predicted by a test to be positive, and may be calculated as the number of correctly identified cancer samples divided by the total number of cancer samples. Specificity is a measure of the “true negatives” that are predicted by a test to be negative, and may be calculated as the number of correctly identified normal samples divided by the total number of normal samples. AUC is a measure of the area under a Receiver Operating Characteristic curve, which is a plot of sensitivity vs. the false positive rate (1-specificity). The greater the AUC, the more powerful the predictive value of the test. Other useful measures of the utility of a test include the “positive predictive value,” which is the percentage of actual positives who test as positives, and the “negative predictive value,” which is the percentage of actual negatives who test as negatives. In some embodiments, the level of one or more nullomers in samples obtained from subjects having different cancer statuses show a statistically significant difference of at least about 0.05 (p=0.05) relative to normal subjects, as determined relative to a suitable control. In some embodiments, the level of one or more nullomers in samples obtained from subjects having different cancer statuses show a statistically significant difference of at least about 0.01 (p=0.01) relative to normal subjects, as determined relative to a suitable control. In some embodiments, the level of one or more nullomers in samples obtained from subjects having different cancer statuses show a statistically significant difference of at least about 0.005 (p=0.005) relative to normal subjects, as determined relative to a suitable control. In some embodiments, the level of one or more nullomers in samples obtained from subjects having different cancer statuses show a statistically significant difference of at least about 0.001 (p=0.001) relative to normal subjects, as determined relative to a suitable control.

In other embodiments, diagnostic tests that use nullomers described herein individually or in combination show an accuracy of at least about 75%, e.g., an accuracy of at least about 75%, about 80%, about 85%, about 90%, about 95%, about 97%, about 99% or about 100%. In other embodiments, diagnostic tests that use nullomers described herein individually or in combination show a specificity of at least about 75%, e.g., a specificity of at least about 75%, about 80%, about 85%, about 90%, about 95%, about 97%, about 99% or about 100%. In other embodiments, diagnostic tests that use nullomers described herein individually or in combination show a sensitivity of at least about 75%, e.g., a sensitivity of at least about 75%, about 80%, about 85%, about 90%, about 95%, about 97%, about 99% or about 100%. In other embodiments, diagnostic tests that use nullomers described herein individually or in combination show a specificity and sensitivity of at least about 75% each, e.g., a specificity and sensitivity of at least about 75%, about 80%, about 85%, about 90%, about 95%, about 97%, about 99% or about 100% (for example, a specificity of at least about 80% and sensitivity of at least about 80%, or for example, a specificity of at least about 80% and sensitivity of at least about 95%).

Each nullomer listed in TABLE 1 is identified as being associated with certain type(s) of cancer as provided. In some instances, one particular nullomer may be associated with more than one types of cancers. In other instances, one particular nullomer may be associated with only one type of cancer.

Each nullomer listed in TABLE 1 is differentially present in biological samples derived from subjects having certain types of cancers as compared with normal subjects, and thus each is individually useful in facilitating the determination of those types of cancer in a test subject. Such a method involves determining the level of the nullomer in a sample obtained from the subject. Determining the level of the nullomer in a sample may include measuring, detecting, or assaying the level of the nullomer in the sample using any suitable method, for example, the methods set forth herein. Determining the level of the nullomer in a sample may also include examining the results of an assay that measured, detected, or assayed the level of the nullomer in the sample. The method may also involve comparing the level of the nullomer in a sample with a suitable control. A change in the level of the nullomer relative to that in a normal subject as assessed using a suitable control is indicative of the cancer status or stage of the subject. A diagnostic amount of a nullomer that represents an amount of the nullomer above or below which a subject is classified as having a particular cancer status or stage can be used. For example, if the nullomer is upregulated in samples from an individual having cancer as compared to a normal individual, a measured amount above the diagnostic cutoff provides a diagnosis of the type of cancer that individual has. Generally, the nullomers in TABLE 1 and Table 7 are upregulated in cancer samples relative to samples obtained from normal individuals. As is well-understood in the art, adjusting the particular diagnostic cut-off used in an assay allows one to adjust the sensitivity and/or specificity of the diagnostic assay as desired. The particular diagnostic cut-off can be determined, for example, by measuring the amount of the nullomer in a statistically significant number of samples from subjects with different cancer statuses, and drawing the cut-off at the desired level of accuracy, sensitivity, and/or specificity. In certain embodiments, the diagnostic cut-off can be determined with the assistance of a classification algorithm, as described elsewhere herein.

Accordingly, methods are provided for diagnosing cancer in a subject, by determining the level of at least one nullomer in a sample from the subject, wherein a difference in the level of the at least one nullomer versus that in a normal subject (as determined relative to a suitable control) is indicative of cancer in the subject. In some embodiments, the at least one nullomer includes one or more nullomers from TABLE 1. In some embodiments, a difference in the level of the at least one nullomer versus that in a normal subject (as determined relative to a suitable control) is indicative of the type(s) of cancer identified as being associated with the detected at least one nullomer in the subject. For example, the disclosed method of determining the level of at least one nullomer in a sample from a subject, wherein an increase in the level of the at least one nullomer relative to a control is indicative of cancer in the subject, particularly of the type(s) of cancer identified as being associated with the at least one nullomer detected. In some embodiments, the subject is diagnosed with having breast cancer, pancreatic cancer, esophagus cancer, lymphoid cancer, kidney cancer, ovary cancer, head and neck cancer, lung cancer, stomach cancer, CNS cancer, uterus cancer, skin cancer, colorectal cancer, prostate cancer, bladder cancer, bone and soft tissue cancer, biliary cancer, cervix cancer, thyroid cancer, myeloid cancer, or liver cancer by the disclosed method.

Optionally, the method may further comprise providing a diagnosis that the subject has or does not have cancer based on the level of at least one nullomer in the sample. In addition or alternatively, the method may further comprise correlating a difference in the level or levels of at least one nullomer relative to a suitable control with a diagnosis of cancer in the subject. In some embodiments, such a diagnosis may be provided directly to the subject, or it may be provided to another party involved in the subject's care.

While individual nullomers are useful in diagnostic applications for various types of cancer, as shown herein, a combination of nullomers may provide greater predictive value of cancer status or stage than the nullomers when used alone. Specifically, the detection of a plurality of nullomers can increase the accuracy, sensitivity, and/or specificity of a diagnostic test. The detection of a plurality of nullomers can also assist in narrowing down the type of cancer and/or status or stage thereof in a subject. This is particular useful when a given nullomer is identified as being associated with more than one type of cancer. For instance, if nullomer A is identified as being associated with cancers X, Y and Z, nullomer B is identified as being associated with cancers X and Y, and nullomer C is identified as being associated with cancers X and Z, by a process of elimination, a detection of the presence of nullomers A, B and C in a subject is indicative that the subject has cancer X. The disclosure thus includes the individual nullomer provided in TABLE 1 and nullomer combinations as set forth herein, and their use in methods and kits described herein. Accordingly, methods are provided for diagnosing cancer in a subject, by determining the level of two or more nullomers in a sample from the subject, wherein a difference in the level of the nullomers versus that in a normal subject (as determined relative to a suitable control) is indicative of cancer in the subject. In some embodiments, the nullomers include one or more of nullomers provided in TABLE1. In some embodiments, the type(s) of cancer thus diagnosed is/are the one(s) provided in TABLE 1 as being associated with each individual nullomer provided in TABLE 1.

Also provided is a method of diagnosing cancer in a subject by determining the levels of two or more nullomers in a sample from the subject, comparing the levels of the two or more nullomers in the sample to a set of data representing levels of the nullomers present in normal subjects and subjects having a particular type of cancer, and diagnosing the subject as having or not having that particular type of cancer based on the comparison. In such a method, the set of data serves as a suitable control or reference standard for comparison with the sample from the subject.

Comparison of the sample from the subject with the set of data may be assisted by a classification algorithm, which computes whether or not a statistically significant difference exists between the collective levels of the two or more nullomers in the sample, and the levels of the same nullomers present in normal subjects or subjects having cancer.

### Generation of Classification Algorithms for Qualifying Cancer Type and Status

In some embodiments, data that are generated using samples such as “known samples” can then be used to “train” a classification model. A “known sample” is a sample that has been pre-classified, e.g., classified as being derived from a normal subject or from a subject having a particular type of cancer. The data that are derived from the spectra and are used to form the classification model can be referred to as a “training data set.” Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased versus non-diseased).

In some embodiments, data for the training data set that is used to form the classification model can be obtained directly from quantitative PCR (for example, Ct values obtained using the double delta Ct method), or from high-throughput expression profiling, such as microarray analysis (for example, total counts or normalized counts from a nullomer expression assay).

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review,” IEEE Transactions on Pattern Analysis and Machine Intelligence, Vol. 22, No. 1, January 2000, the teachings of which are incorporated by reference in its entirety.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (support vector machines).

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm. Learning algorithms asserted for use in classifying biological information are described, for example, in PCT International Publication No. WO 01/31580 (Barnhill et al., “Methods and devices for identifying patterns in biological systems and methods of use thereof”), U.S. application publication No. 2002/0193950 A1 (Gavin et al, “Method or analyzing mass spectra”), U.S. application publication No. 2003/0004402 A1 (Hitt et al., “Process for discriminating between biological states based on hidden patterns from biological data”), and U.S. application publication No. 2003/0055615 A1 (Zhang and Zhang, “Systems and methods for processing biological expression data”). The contents of the foregoing patent applications are incorporated herein by reference in their entireties.

The classification models can be formed on and used on any suitable digital computer. Suitable digital computers include micro, mini, or large computers using any standard or specialized operating system, such as a Unix, WINDOWS or LINUX based operating system.

The training data set(s) and the classification models can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

The learning algorithms described herein can be used for developing classification algorithms for nullomers for various types of cancer. The classification algorithms can, in turn, be used in diagnostic tests by providing diagnostic values (e.g., cut-off points) for nullomers used singly or in combination.

### Additional Diagnostic Tests

The level of nullomers indicative of various types of cancer may be used as a stand-alone diagnostic indicator of cancer in a subject. Optionally, the methods may include the performance of at least one additional test to facilitate the diagnosis of cancer. For example, other tests in addition to determining the level of one or more nullomers in order to facilitate a diagnosis of cancer may be performed. Any other test or combination of tests used in clinical practice to facilitate a diagnosis of cancer may be used in conjunction with the nullomers described herein.

### Methods of Treatment

In some embodiments, where a subject is diagnosed with a particular type of cancer by the methods described herein, the disclosure further provides methods of treating the subject identified as having a cancer. Accordingly, in some embodiments, the disclosure relates to a method of treating cancer in a subject, comprising determining the level of at least one nullomer in a sample from the subject, wherein a difference in the level of at least one nullomer versus that in a normal subject as determined relative to a suitable control is indicative of cancer in the subject, and administering a therapeutically effective amount of a cancer therapeutic to the subject. In another embodiments, the disclosure relates to a method of treating a subject having cancer, comprising identifying a subject having cancer in which the level of at least one nullomer in a sample from the subject is different (e.g., increased) versus that in a normal subject as determined relative to a suitable control, and administering a therapeutically effective amount of a cancer therapeutic to the subject.

The term “cancer therapeutic” includes, for example, substances approved by the U.S. Food and Drug Administration for the treatment of cancer. For instance, drugs approved to treat breast cancer include, but are not limited to, Abemaciclib, Abitrexate (Methotrexate), Abraxane (Paclitaxel Albumin-stabilized Nanoparticle Formulation), Ado-Trastuzumab Emtansine, Afinitor (Everolimus), Anastrozole, Aredia (Pamidronate Disodium), Arimidex (Anastrozole), Aromasin (Exemestane), Capecitabine, Clafen (Cyclophosphamide), Cyclophosphamide, Cytoxan (Cyclophosphamide), Docetaxel, Doxorubicin Hydrochloride, Ellence (Epirubicin Hydrochloride), Epirubicin Hydrochloride, Eribulin Mesylate, Everolimus, Exemestane, 5-FU (Fluorouracil Injection), Fareston (Toremifene), Faslodex (Fulvestrant), Femara (Letrozole), Fluorouracil Injection, Folex (Methotrexate), Folex PFS (Methotrexate), Fulvestrant, Gemcitabine Hydrochloride, Gemzar (Gemcitabine Hydrochloride), Goserelin Acetate, Halaven (Eribulin Mesylate), Herceptin (Trastuzumab), Ibrance (Palbociclib), Ixabepilone, Ixempra (Ixabepilone), Kadcyla (Ado-Trastuzumab Emtansine), Kisqali (Ribociclib), Lapatinib, Ditosylate, Letrozole, Megestrol Acetate, Methotrexate, Methotrexate LPF (Methotrexate), Mexate (Methotrexate), Mexate-AQ (Methotrexate), Neosar (Cyclophosphamide), Neratinib Maleate, Nerlynx (Neratinib Maleate), Nolvadex (Tamoxifen Citrate), Paclitaxel, Paclitaxel Albumin-stabilized Nanoparticle Formulation, Palbociclib, Pamidronate Disodium, Perjeta (Pertuzumab), Pertuzumab, Ribociclib, Tamoxifen Citrate, Taxol (Paclitaxel), Taxotere (Docetaxel), Thiotepa, Toremifene, Trastuzumab, Tykerb (Lapatinib Ditosylate), Velban (Vinblastine Sulfate), Velsar (Vinblastine Sulfate), Verzenio (Abemaciclib), Vinblastine Sulfate, Xeloda (Capecitabine), Zoladex (Goserelin Acetate).

The cancer therapeutics may be administered to a subject using a pharmaceutical composition. Suitable pharmaceutical compositions comprise a pharmaceutically effective amount of a cancer therapeutic (or a pharmaceutically acceptable salt or ester thereof), and optionally comprise a pharmaceutically acceptable carrier. In certain embodiments, these compositions optionally further comprise one or more additional therapeutic agents.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the final isolation and purification of the compounds, or separately by reacting a free base or free acid function with a suitable reagent. For example, a free base function can be reacted with a suitable acid. Furthermore, where the compounds carry an acidic moiety, suitable pharmaceutically acceptable salts thereof may, include metal salts such as alkali metal salts, e.g., sodium or potassium salts, and alkaline earth metal salts, e.g., calcium or magnesium salts. In some embodiments, the cancer therapeutic is a pharmaceutically acceptable salt.

The term “pharmaceutically acceptable ester,” as used herein, refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. In some embodiments, the cancer therapeutic is a pharmaceutically acceptable ester.

As described above, the pharmaceutical compositions may additionally comprise a pharmaceutically acceptable carrier. The term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, suitable for preparing the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogenfree water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Compositions for use in the present disclosure may be formulated to have any concentration of the cancer therapeutic desired. In some embodiments, the composition is formulated such that it comprises a therapeutically effective amount of the cancer therapeutic.

The disclosure generally relates to a method of diagnosing a subject with a benign, pre-malignant, or malignant hyperproliferative cell comprising: detecting the presence, absence, and/or quantity of at least one nullomer in a sample. In some embodiments, the step of detecting comprise exposing a sample from a subject (e.g., a human subject) to one or a plurality of probes, each probe capable of binding one or a plurality of nullomer in the sample. In some embodiments, the probe is a labeled nucleic acid molecule (DNA, RNA or hybrid thereof) that comprises at least about 70%, 80%, 85%, 90%, 91%, 92%, 93%, 94%, 95%, 96%, 97%, 98%, 99% or 100% sequence identity to the complement of any nucleic acid sequences of TABLE 1 or TABLE 7. In some embodiments, the probe is a labeled nucleic acid molecule (DNA, RNA or hybrid thereof) that is an RNA sequence comprising at least about 70%, 80%, 85%, 90%, 91%, 92%, 93%, 94%, 95%, 96%, 97%, 98%, 99% or 100% sequence identity to the complement of any nucleic acid sequences of TABLE 1, where each thymine is replaced with a uracil. In some embodiments, the plurality of probes are one or a combination of labeled nucleic acid sequences that are an RNA complementary to a nucleic acid sequence comprising at least about 70%, 80%, 85%, 90%, 91%, 92%, 93%, 94%, 95%, 96%, 97%, 98%, 99% or 100% sequence identity to any nucleic acid sequences of TABLE 1 or Table 7. In some embodiments, the plurality of probes are one or a combination of labeled nucleic acid sequences chosen from any nucleic acid sequences of TABLE 7. In some embodiments, the plurality of probes comprise one or a combination of nucleic acid sequences complementary to the nucleic acid sequences chosen from any nucleic acid sequences of TABLE 7. In some embodiments, the probe is a labeled nucleic acid molecule (DNA, RNA or hybrid thereof) that comprises at least about 70%, 80%, 85%, 90%, 91%, 92%, 93%, 94%, 95%, 96%, 97%, 98%, 99% or 100% sequence identity to the complement of any nucleic acid sequences of TABLE 7. In some embodiments, the probe is a labeled nucleic acid molecule (DNA, RNA or hybrid thereof) that is an RNA sequence comprising at least about 70%, 80%, 85%, 90%, 91%, 92%, 93%, 94%, 95%, 96%, 97%, 98%, 99% or 100% sequence identity to the complement of any nucleic acid sequences of TABLE 7, where each thymine is replaced with a uracil. In some embodiments, the plurality of probes are one or a combination of labeled nucleic acid sequences that are an RNA complementary to a nucleic acid sequence comprising at least about 70%, 80%, nucleic acid sequences of TABLE 7. In some embodiments, the plurality of probes are one or a combination of labeled nucleic acid sequences chosen from any nucleic acid sequences of TABLE 7. In some embodiments, the plurality of probes comprise one or a combination of nucleic acid sequences complementary to the nucleic acid sequences chosen from any nucleic acid sequences of TABLE 7.

In some embodiments, the probe is a labeled nucleic acid molecule (DNA, RNA or hybrid thereof) that comprises at least about 70%, 80%, 85%, 90%, 91%, 92%, 93%, 94%, 95%, 96%, 97%, 98%, 99% or 100% sequence identity to the complement of any nucleic acid sequences of TABLE 1.

In some embodiments, the probe is a labeled nucleic acid molecule (DNA, RNA or hybrid thereof) that is an RNA sequence comprising at least about 70%, 80%, 85%, 90%, 91%, 92%, 93%, 94%, 95%, 96%, 97%, 98%, 99% or 100% sequence identity to the complement of any nucleic acid sequences of TABLE 4, where each thymine is replaced with a uracil. In some embodiments, the plurality of probes are one or a combination of labeled nucleic acid sequences that are an RNA complementary to a nucleic acid sequence comprising at least about 70%, 80%, 85%, 90%, 91%, 92%, 93%, 94%, 95%, 96%, 97%, 98%, 99% or 100% sequence identity to any nucleic acid sequences of TABLE 4. In some embodiments, the plurality of probes are one or a combination of labeled nucleic acid sequences chosen from any nucleic acid sequences of TABLE 4. In some embodiments, the plurality of probes comprise one or a combination of nucleic acid sequences complementary to the nucleic acid sequences chosen from any nucleic acid sequences of TABLE 4.

In some embodiments, the probe is a labeled nucleic acid molecule (DNA, RNA or hybrid thereof) that comprises at least about 70%, 80%, 85%, 90%, 91%, 92%, 93%, 94%, 95%, 96%, 97%, 98%, 99% or 100% sequence identity to the complement of any nucleic acid sequences of TABLE 5. In some embodiments, the probe is a labeled nucleic acid molecule (DNA, RNA or hybrid thereof) that is an RNA sequence comprising at least about 70%, 80%, 85%, 90%, 91%, 92%, 93%, 94%, 95%, 96%, 97%, 98%, 99% or 100% sequence identity to the complement of any nucleic acid sequences of TABLE 5, where each thymine is replaced with a uracil. In some embodiments, the plurality of probes are one or a combination of labeled nucleic acid sequences that are an RNA complementary to a nucleic acid sequence comprising at least about 70%, 80%, nucleic acid sequences of TABLE 5. In some embodiments, the plurality of probes are one or a combination of labeled nucleic acid sequences chosen from any nucleic acid sequences of TABLE 5. In some embodiments, the plurality of probes comprise one or a combination of nucleic acid sequences complementary to the nucleic acid sequences chosen from any nucleic acid sequences of TABLE 5.

In any of the disclosed method embodiments, the subject may be a human diagnosed with or suspected as having cancer. In any of the disclosed method embodiments, wherein the step of detecting is preceded by a step of acquiring a sample from the subject.

In some embodiments, the probe or plurality of probes are one or a plurality of antibodies or antibody fragments comprising a CDR that binds to a nucleic acid molecule (DNA, RNA or hybrid thereof) that comprises at least 70%, 80%, 85%, 90%, 91%, 92%, 93%, 94%, 95%, 96%, 97%, 98%, 99% or 100% sequence identity to any nucleic acid sequences of TABLE 1. In some embodiments, the probe or plurality of probes are one or a plurality of antibodies or antibody fragments comprising a CDR that binds to a nucleic acid molecule (DNA, RNA or hybrid thereof) that comprises at least about 70%, 80%, 85%, 90%, 91%, 92%, 93%, 94%, 95%, 96%, 97%, 98%, 99% or 100% sequence identity to any nucleic acid sequences of TABLE 1, wherein each of sequences are modified such that the thymines in each sequence are replaced with a uracil. In some of the embodiments, the methods further comprise isolating RNA from the sample before exposing the sample to one or a plurality of probes. In some embodiments, the method comprises detecting or quantifying an amount of nullomers in a sample by performing semiquantitative or quantitative PCR or sequencing analysis of the nullomers in a sample. Probes may be immobilized to a solid support such as an ELISA plate, plastic, slide, microarray, silica chip or other surface such that the single-strand nucleotide sequences are exposed to a sample comprising nullomers from a subject. The probes may comprise, in some embodiments, from about 5 to about 100 nucleotides in length and comprise any of the sequences provided in TABLE 1 or any complementary sequence in RNA or DNA form of the sequences set forth in TABLE 1. In any of the disclosed method embodiments, the step of detecting the presence, absence, and/or quantity of at least one nullomer having at least about 70% sequence identity to one of the nullomers in a sample comprises using a chemoluminescent probe, fluorescent probe, and/or fluorescence microscopy, calculating the presence or quantity by correlating the signal of the detectable probe to the presence of the nullomer.

In some embodiments, any of the methods disclosed herein further comprise a step of correlating the presence or quantity of one or more nullomers, such as those disclosed in TABLE 1 or any combination thereof, to the likelihood that the subject has cancer. In some embodiments, the disclosure relates to a method of preparing, isolating or assessing a nucleic acid or ribonucleic acid fraction from a subject useful for analyzing a nullomer involved in cancer comprising: extracting DNA or RNA from a substantially cell-free sample of blood plasma or blood serum of a subject to obtain DNA or RNA pools; (b) producing a fraction of the DNA or RNA extracted in (a) by: (i) sequence discrimination of the DNA or RNA; and (ii) selectively removing nullomers by exposing one or a plurality of probes to the nullomers, wherein the nullomers after (b) comprises one or a plurality of nullomers disclosed in TABLE 1; and (c) analyzing the nullomers in the fraction of DNA or RNA produced in (b). In some embodiments, the step of analyzing comprises normalizing the amount of nullomers in the sample as compared to a control amount of nullomers from a control sample and determining whether the subject has cancer by comparing the normalized presence, absence or quantity of nullomers in the sample to the presence, absence or quantity of nullomers in a control sample.

### Kits for Detection of Nullomers

The disclosure also provides kits for diagnosing type of cancer, tissue of origin, and status or stage of the cancer in a subject, which kits are useful for determining the level of one or more nullomers from TABLE 1, wherein the sequences optionally comprise uracils in place of one, more than one, or all of the disclosed thymines), and combinations thereof. In some embodiments, the one or more nullomers are selected from the nullomers listed in TABLE 1. Kits may include materials and reagents adapted to selectively detect the presence of a nullomer or group of nullomers diagnostic for cancer in a sample of a subject. For example, in some embodiments, the kit may include a reagent that specifically hybridizes to a nullomer. Such a reagent may be a nucleic acid molecule in a form suitable for detecting the nullomer, for example, a probe or a primer. The kit may include reagents useful for performing an assay to detect one or more nullomers, for example, reagents which may be used to detect one or more nullomers in a qPCR reaction. The kit may likewise include a microarray useful for detecting one or more nullomers.

In some embodiments, the kit may contain instructions for suitable operational parameters in the form of a label or product insert. For example, the instructions may include information or directions regarding how to collect a sample, how to determine the level of one or more nullomers in a sample, and/or how to correlate the level of one or more nullomers in a sample with the type of cancer, tissue of origin, and status or stage of the cancer of a subject.

In some embodiments, the kit can contain one or more containers with nullomer samples, to be used as reference standards, suitable controls, or for calibration of an assay to detect the nullomers in a test sample.

TABLE 2
Radioisotopes that may be incorporated into pharmaceutical compositions or
used as probes or labels with nullomers.
²H, ³H, ¹³C, ¹⁴C, ¹⁵N, ¹⁶O, ¹⁷O, ³¹P, ³²p, ³⁵S, ¹⁸F, ³⁶C1, ²²⁵Ac, ²²⁷Ac, ₂₁₂Bi, ₂₁₃Bi, ₁₀₉Cd, ₆₀Co,
⁶⁴Cu, ⁶⁷Cu, ¹⁶⁶Dy, ¹⁶⁹Er, ¹⁵²Eu, ¹⁵⁴Eu, ¹⁵³Gd, ¹⁹⁸Au, ¹⁶⁶Ho, ¹²⁵I, ¹³¹I, ¹⁹²Ir, ¹⁷⁷Lu, ⁹⁹Mo, ¹⁹⁴Os, ¹⁰³Pd,
^{195m}pt, ³²P, ³³P, ²²³Ra, ¹⁸⁶Re, ¹⁸⁸Re, ¹⁰⁵Rh, ¹⁴⁵Sm, ¹⁵³Sm, ⁴⁷Sc, ⁷⁵Se, ⁸⁵Sr, ⁸⁹Sr, ^{99m}Tc, ²²⁸Th, ²²⁹Th,
¹⁷⁰Tm, ^{117m}Sn, ¹⁸⁸W, ¹²⁷Xe, ¹⁷⁵Yb, ⁹⁰Y, ⁹¹Y

TABLE 3
Table of chemotherapeutic agents.
Alkylating agents
Cyclophosphamide
Mechlorethamine
Chlorambucil
Melphalan
Anthracyclines
Daunorubicin
Doxorubicin
Epirubicin
Idarubicin
Mitoxantrone
Valrubicin
Cytoskeletal disruptors (Taxanes)
Paclitaxel
Docetaxel
Epothilones
Histone Deacetylase Inhibitors
Vorinostat
Romidepsin
Inhibitors of Topoisomerase I
Irinotecan
Topotecan
Inhibitors of Topoisomerase II
Etoposide
Teniposide
Tafluposide
Kinase inhibitors
Bortezomib
Erlotinib
Gefitinib
Imatinib
Vemurafenib
Vismodegib
Monoclonal antibodies
Bevacizumab
Cetuximab
Ipilimumab
Ofatumumab
Ocrelizumab
Panitumab
Rituximab
Nucleotide analogs and precursor analogs
Azacitidine
Azathioprine
Capecitabine
Cytarabine
Doxifluridine
Fluorouracil
Gemcitabine
Hydroxyurea
Mercaptopurine
Methotrexate
Tioguanine (formerly Thioguanine)
Peptide antibiotics
Bleomycin
Actinomycin
Platinum-based agents
Carboplatin
Cisplatin
Oxaliplatin
Retinoids
Tretinoin
Alitretinoin
Bexarotene
Vinca alkaloids and derivatives
Vinblastine
Vincristine
Vindesine
Vinorelbine
Actinomycin
All-trans retinoic acid
Azacitidine
Azathioprine
Bleomycin
Bortezomib
Carboplatin
Capecitabine
Cisplatin
Chlorambucil
Cyclophosphamide
Cytarabine
Daunorubicin
Docetaxel
Doxifluridine
Doxorubicin
Epirubicin
Epothilone
Etoposide
Fluorouracil
Gemcitabine
Hydroxyurea
Idarubicin
Imatinib
Irinotecan
Mechlorethamine
Mercaptopurine
Methotrexate
Mitoxantrone
Oxaliplatin
Paclitaxel
Pemetrexed
Teniposide
Tioguanine
Topotecan
Valrubicin
Vinblastine
Vincristine
Vindesine
Vinorelbine

### Systems

In some methods of treatment disclosed herein, the agent in selected from one or a plurality of agents chosen from Table 3.

The above-described methods can be implemented in any of numerous ways. For example, the embodiments may be implemented using a computer program product (i.e. software), hardware, software or a combination thereof. When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

A computer employed to implement at least a portion of the functionality described herein may include a memory, coupled to one or more processing units (also referred to herein simply as “processors”), one or more communication interfaces, one or more display units, and one or more user input devices. The memory may include any computer-readable media, and may store computer instructions (also referred to herein as “processor-executable instructions”) for implementing the various functionalities described herein. The processing unit(s) may be used to execute the instructions. The communication interface(s) may be coupled to a wired or wireless network, bus, or other communication means and may therefore allow the computer to transmit communications to and/or receive communications from other devices. The display unit(s) may be provided, for example, to allow a user to view various information in connection with execution of the instructions. The user input device(s) may be provided, for example, to allow the user to make manual adjustments, make selections, enter data or various other information, and/or interact in any of a variety of manners with the processor during execution of the instructions.

The various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the invention disclosed herein. The computer readable medium or media can be transportable, such that the program or programs stored thereon can be loaded onto one or more different computers or other processors to implement various aspects of the present invention as discussed above. In some embodiments, the system comprises cloud-based software that executes one or all of the steps of each disclosed method instruction.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present disclosure need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Also, the disclosure relates to various embodiments in which one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

In some embodiments, the disclosure relates to a system that comprises at least one processor, a program storage, such as memory, for storing program code executable on the processor, and one or more input/output devices and/or interfaces, such as data communication and/or peripheral devices and/or interfaces. In some embodiments, the user device and computer system or systems are communicably connected by a data communication network, such as a Local Area Network (LAN), the Internet, or the like, which may also be connected to a number of other client and/or server computer systems. The user device and client and/or server computer systems may further include appropriate operating system software.

In some embodiments, components and/or units of the devices described herein may be able to interact through one or more communication channels or mediums or links, for example, a shared access medium, a global communication network, the Internet, the World Wide Web, a wired network, a wireless network, a combination of one or more wired networks and/or one or more wireless networks, one or more communication networks, an a-synchronic or asynchronous wireless network, a synchronic wireless network, a managed wireless network, a non-managed wireless network, a burstable wireless network, a non-burstable wireless network, a scheduled wireless network, a non-scheduled wireless network, or the like.

Discussions herein utilizing terms such as, for example, “processing,” “computing,” “calculating,” “determining,” or the like, may refer to operation(s) and/or process(es) of a computer, a computing platform, a computing system, or other electronic computing device, that manipulate and/or transform data represented as physical (e.g., electronic) quantities within the computer's registers and/or memories into other data similarly represented as physical quantities within the computer's registers and/or memories or other information storage medium that may store instructions to perform operations and/or processes.

Some embodiments may take the form of an entirely hardware embodiment, an entirely software embodiment, or an embodiment including both hardware and software elements. Some embodiments may be implemented in software, which includes but is not limited to firmware, resident software, microcode, or the like.

Furthermore, some embodiments may take the form of a computer program product accessible from a computer-usable or computer-readable medium providing program code for use by or in connection with a computer or any instruction execution system. For example, a computer-usable or computer-readable medium may be or may include any apparatus that can contain, store, communicate, propagate, or transport the program for use by or in connection with the instruction execution system, apparatus, or device.

In some embodiments, the medium may be or may include an electronic, magnetic, optical, electromagnetic, InfraRed (IR), or semiconductor system (or apparatus or device) or a propagation medium. Some demonstrative examples of a computer-readable medium may include a semiconductor or solid state memory, magnetic tape, a removable computer diskette, a Random Access Memory (RAM), a Read-Only Memory (ROM), a rigid magnetic disk, an optical disk, or the like. Some demonstrative examples of optical disks include Compact Disk-Read-Only Memory (CD-ROM), Compact Disk-Read/Write (CD-R/W), DVD, or the like.

In some embodiments, a data processing system suitable for storing and/or executing program code may include at least one processor coupled directly or indirectly to memory elements, for example, through a system bus. The memory elements may include, for example, local memory employed during actual execution of the program code, bulk storage, and cache memories which may provide temporary storage of at least some program code in order to reduce the number of times code must be retrieved from bulk storage during execution.

In some embodiments, input/output or I/O devices (including but not limited to keyboards, displays, pointing devices, etc.) may be coupled to the system either directly or through intervening I/O controllers. In some embodiments, network adapters may be coupled to the system to enable the data processing system to become coupled to other data processing systems or remote printers or storage devices, for example, through intervening private or public networks. In some embodiments, modems, cable modems and Ethernet cards are demonstrative examples of types of network adapters. Other suitable components may be used.

Some embodiments may be implemented by software, by hardware, or by any combination of software and/or hardware as may be suitable for specific applications or in accordance with specific design requirements. Some embodiments may include units and/or sub-units, which may be separate of each other or combined together, in whole or in part, and may be implemented using specific, multi-purpose or general processors or controllers. Some embodiments may include buffers, registers, stacks, storage units and/or memory units, for temporary or long-term storage of data or in order to facilitate the operation of particular implementations.

Some embodiments may be implemented, for example, using a machine-readable medium or article which may store an instruction or a set of instructions that, if executed by a machine, cause the machine to perform a method steps and/or operations described herein. Such machine may include, for example, any suitable processing platform, computing platform, computing device, processing device, electronic device, electronic system, computing system, processing system, computer, processor, or the like, and may be implemented using any suitable combination of hardware and/or software. The machine-readable medium or article may include, for example, any suitable type of memory unit, memory device, memory article, memory medium, storage device, storage article, storage medium and/or storage unit; for example, memory, removable or non-removable media, erasable or non-erasable media, writeable or re-writeable media, digital or analog media, hard disk drive, floppy disk, Compact Disk Read Only Memory (CD-ROM), Compact Disk Recordable (CD-R), Compact Disk Re-Writeable (CD-RW), optical disk, magnetic media, various types of Digital Versatile Disks (DVDs), a tape, a cassette, or the like. The instructions may include any suitable type of code, for example, source code, compiled code, interpreted code, executable code, static code, dynamic code, or the like, and may be implemented using any suitable high-level, low-level, object-oriented, visual, compiled and/or interpreted programming language, e.g., C, C++, Java™, BASIC, Pascal, Fortran, Cobol, assembly language, machine code, or the like.

Many of the functional units described in this specification have been labeled as circuits, in order to more particularly emphasize their implementation independence. For example, a circuit may be implemented as a hardware circuit comprising custom very-large-scale integration (VLSI) circuits or gate arrays, off-the-shelf semiconductors such as logic chips, transistors, or other discrete components. A circuit may also be implemented in programmable hardware devices such as field programmable gate arrays, programmable array logic, programmable logic devices or the like.

In some embodiment, the circuits may also be implemented in machine-readable medium for execution by various types of processors. An identified circuit of executable code may, for instance, comprise one or more physical or logical blocks of computer instructions, which may, for instance, be organized as an object, procedure, or function. Nevertheless, the executables of an identified circuit need not be physically located together, but may comprise disparate instructions stored in different locations which, when joined logically together, comprise the circuit and achieve the stated purpose for the circuit. Indeed, a circuit of computer readable program code may be a single instruction, or many instructions, and may even be distributed over several different code segments, among different programs, and across several memory devices. Similarly, operational data may be identified and illustrated herein within circuits, and may be embodied in any suitable form and organized within any suitable type of data structure. The operational data may be collected as a single data set, or may be distributed over different locations including over different storage devices, and may exist, at least partially, merely as electronic signals on a system or network.

The computer readable medium (also referred to herein as machine-readable media or machine-readable content) may be a tangible computer readable storage medium storing the computer readable program code. The computer readable storage medium may be, for example, but not limited to, an electronic, magnetic, optical, electromagnetic, infrared, holographic, micromechanical, or semiconductor system, apparatus, or device, or any suitable combination of the foregoing. As alluded to above, examples of the computer readable storage medium may include but are not limited to a portable computer diskette, a hard disk, a random access memory (RAM), a read-only memory (ROM), an erasable programmable read-only memory (EPROM or Flash memory), a portable compact disc read-only memory (CD-ROM), a digital versatile disc (DVD), an optical storage device, a magnetic storage device, a holographic storage medium, a micromechanical storage device, or any suitable combination of the foregoing. In the context of this document, a computer readable storage medium may be any tangible medium that can contain, and/or store computer readable program code for use by and/or in connection with an instruction execution system, apparatus, or device.

The computer readable medium may also be a computer readable signal medium. A computer readable signal medium may include a propagated data signal with computer readable program code embodied therein, for example, in baseband or as part of a carrier wave. Such a propagated signal may take any of a variety of forms, including, but not limited to, electrical, electro-magnetic, magnetic, optical, or any suitable combination thereof. A computer readable signal medium may be any computer readable medium that is not a computer readable storage medium and that can communicate, propagate, or transport computer readable program code for use by or in connection with an instruction execution system, apparatus, or device. As also alluded to above, computer readable program code embodied on a computer readable signal medium may be transmitted using any appropriate medium, including but not limited to wireless, wireline, optical fiber cable, Radio Frequency (RF), or the like, or any suitable combination of the foregoing. In one embodiment, the computer readable medium may comprise a combination of one or more computer readable storage mediums and one or more computer readable signal mediums. For example, computer readable program code may be both propagated as an electro-magnetic signal through a fiber optic cable for execution by a processor and stored on RAM storage device for execution by the processor.

Computer readable program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The computer readable program code may execute entirely on a user's computer, partly on the user's computer, as a stand-alone computer-readable package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The program code may also be stored in a computer readable medium that can direct a computer, other programmable data processing apparatus, or other devices to function in a particular manner, such that the instructions stored in the computer readable medium produce an article of manufacture including instructions which implement the function/act specified in the schematic flowchart diagrams and/or schematic block diagrams block or blocks.

Functions, operations, components and/or features described herein with reference to one or more embodiments, may be combined with, or may be utilized in combination with, one or more other functions, operations, components and/or features described herein with reference to one or more other embodiments, or vice versa.

Other embodiments are described in the following non-limiting Examples. Various publications, including patents, published applications, technical articles and scholarly articles are cited throughout the specification. Each of these cited publications is incorporated by reference herein in its entirety.

### EXAMPLES

### Example 1. Nullomers for Cancer Diagnosis

Cancer detection using cell-free DNA (cfDNA) has the potential to significantly improve cancer diagnosis and survival. However, cfDNA diagnostics suffer from several deficiencies, including low tumor cfDNA concentration, sensitivity and technical limitations, in particular for cfDNA methylation analyses. Here, we set out to test whether nullomers could be used as a diagnostic tool to detect cancer in general and also specific subtypes. We first analyzed The Cancer Genome Atlas (TCGA; (“The Cancer Genome Atlas Program” 2018)) database finding recurrent nullomers created by somatic mutations that could be used to detect not only cancer subtypes with higher accuracy than leading methods (Jiao et al. 2020) but also additional cancer features. Further analyses of cfDNA whole-genome sequencing datasets found that these nullomers can also be used to detect cancer subtypes in these data without the need for healthy control samples. Using a targeted nullomer sequencing panel in cfDNA from individuals with prostate cancer and normal controls found nullomer enrichment in cases. Finally, functional assays of prostate cancer associated nullomers show that they have a functional effect on both coding and noncoding sequences. Combined, our results show that nullomers can be used as rapid, sensitive, specific and straightforward cancer diagnosis and also aid in the identification of gene regulatory mutations associated with cancer.

### 1. Methods

i. Computational Characterization of Recurrent Nullomers

The GRCh38 reference assembly of the human genome was used throughout the study. Nullomer extraction was performed for kmer lengths up to 17 base pairs using the same algorithm described in Georgakopoulos-Soares et al., 2020. By definition, the reverse complement of a nullomer will also be a nullomer. Throughout this example when counting nullomers, the reverse complement of nullomer i is also considered unless i is a palindrome. Mutation calling for whole genome sequencing (WGS) tumor samples from 2,575 individuals across 21 tissues (ICGC/TCGA Pan-Cancer Analysis of Whole Genomes Consortium 2020) was performed for substitutions and indels as described in Georgakopoulos-Soares et al., 2019. To filter out common population variants, we obtained variant information from the gnomAD v2 (Karczewski et al. 2020) and annotated all nullomers that were generated due to these variants. We then excluded from our subsequent analyses nullomers that were found in the population with a p-value of 0.05 or higher. For each substitution, a local window was generated with the mutation introduced in the window sequence; each nullomer was then scanned across the window to check whether any matches were found. Matches were reported and stored. Recurrent nullomers were annotated as those that resulted from substitutions or indels across two or more patients within a cancer type. When possible, ri was chosen to get ˜10,000 nullomers from each tissue, otherwise it was set to 2.

ii. Classification

The algorithms to replicate our classification analysis are: TrainNullomerClassifier, which trains a predictive model on cancer tissue based on the nullomer profile and RunNullomerClassifier, which runs the trained classifier (from TrainNullomerClassifier) on a set of nullomers (from FindDNANullomersFromReads).

iii. Comparison to validated neoantigens

We downloaded a list of 1,967 validated neoantigens from biopharm.zju.edu.cn/download.neoantigen/iedb_validated.zip. Requiring predicted strong binding and a positive validation, left us with 1,700 neoantigens. To evaluate the enrichment of neoantigens corresponding to recurrent nullomers, we assumed a hypergeometric distributions with 1,700 draws from an urn with 188,659 white balls (recurrent nullomers) and 186,067,892 black balls (number of non-recurrent nullomers found).

iv. Nullomer Identification in ctDNA Samples

We developed a Poisson model where the expected number of nullomers of type i is given by C′eNi 3, where C is the coverage, e is the error rate, Ni is the number of loci where a substitution could result in the creation of nullomer i, and the division by 3 is to account for the fact that only one of three substitutions will create the nullomer.

v. Targeted Sequencing of cfDNA Samples

We designed a set of 4,590 baits (total of 78,102 bp of sequence space) covering all nullomer locations implicated in our prostate cancer classifier. 309 probes were removed from the panel due to synthesis issues, ending with 4,280 probes (93.24% final coverage) of 120 bp in length spanning evenly the exact location of the nullomer causing mutation. Custom target-enriched DNA libraries were generated by Twist Bioscience and prepared using Twist modular library preparation kits enabled by KAPA HiFi HotStart ReadyMix PCR Kit (Kapa Bioscience). Twist universal adapters were replaced with IDT's xGen UDI-UMI 96 barcodes system (IDT). cfDNA was extracted as described in Chen et. al. (Chen et al., 2021). Briefly, whole blood was collected using PAXgene Blood ccfDNA tubes (Qiagen) and the final extraction step was done using the QIAamp Circulating Nucleic Acid Kit (Qiagen). Extracted DNA was stored at −80° ° C. prior to further analysis. cfDNA was then hybridized and libraries were enriched using Streptavidin C1 beads and the washed material was amplified via 9-14 cycles of PCR. Targeted sequencing was performed with PE150 reads, dual index (8 bp i5 and 8 bp+9 bp UMI at i7 position) on a HiSeq4000 platform (Illumina).

vi. Promoter Luciferase Assays

Promoter sequences with and without the nullomer were synthetically generated and cloned into the modified Promega promoter assay luciferase vector pGL4.11b (a gift from Dr. Rick Myers, HudsonAlpha) by BioMatik Inc. and Sanger sequence verified. LNCaP cells were plated at an initial density of 2×10⁵ cells/well in 24-well tissue culture plates and maintained in RPMI medium, 10% FBS supplemented with L-Glutamine and Penicillin/Streptomycin. Plasmids together with a renilla expressing plasmid, pGL4.74 (Promega), at a ratio of 10:1 luciferase:renilla were transfected using the X-tremeGENE™ HP DNA Transfection Reagent (Roche) using 1:4 ratio of DNA (μg) to reagent (μl). 72 hours post transfection, luciferase and renilla levels were measured using the Dual-Luciferase Reporter Assay System (Promega) following the manufacturer's protocol using a GloMax Explorer Multimode Microplate Reader (Promega). Luciferase activity was normalized to renilla levels and presented as Relative Luciferase Units (RLU). Statistical analysis was performed using Prism version 9.0.2 (GraphPad). All values were reported as means (AVG) and standard errors (SE). p values<0.05 were considered statistically significant.

vii. Software Availability

We generated an easy to use software package that enables performing nullomer cancer analyses from sequence-based datasets. The package is composed of six functions: 1) EnumerateNullomers, which extracts all nullomers of specified kmer lengths in a FASTA sample; 2) ExtractMutationNullomers, which finds all mutations that cause the resurfacing of a list of nullomers; 3) IdentifyRecurrentNullomers, which identifies nullomers that recur in a dataset through mutagenesis; 4) FindAlmostNullomers, which identifies the positions that can create a list of nullomers genome-wide for every possible substitution and single base-pair insertion and deletion; 5) FindNullomerVariants, which removes nullomers that are likely to result from common variants in a user specified variant VCF file; and 6) FindDNANullomersFromReads, which performs the identification of nullomers in raw read samples.

The package can be found at: github.com/Ahituv-lab/Nullomerator and a readthedocs tutorial provides in depth details on how to run the software functions.

### 2. Results

i. Annotation of Mutations that Lead to Nullomers

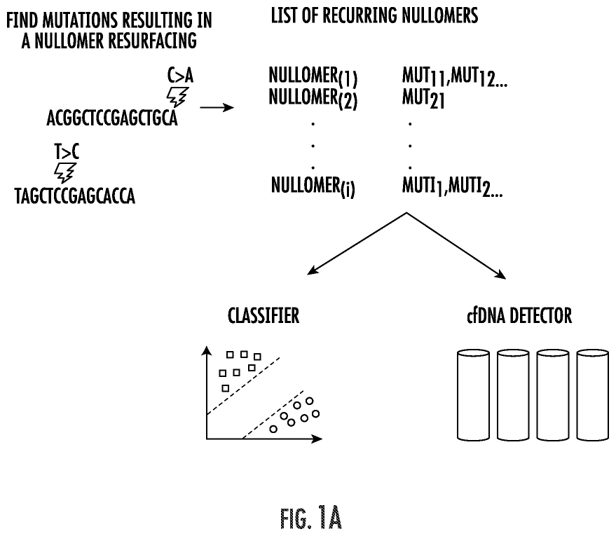

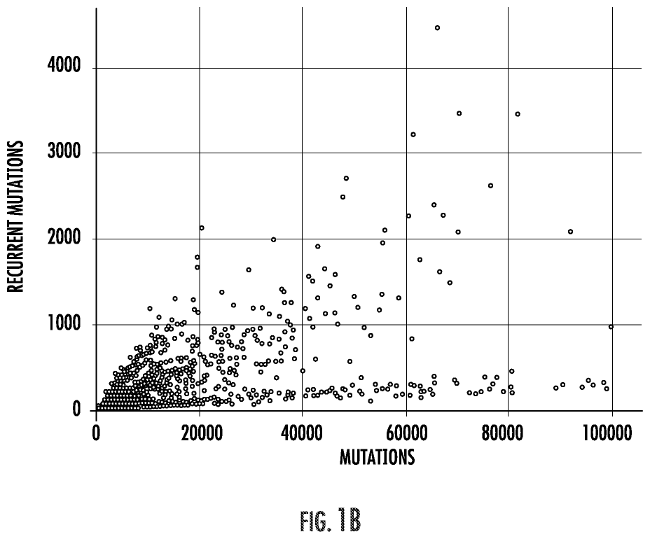

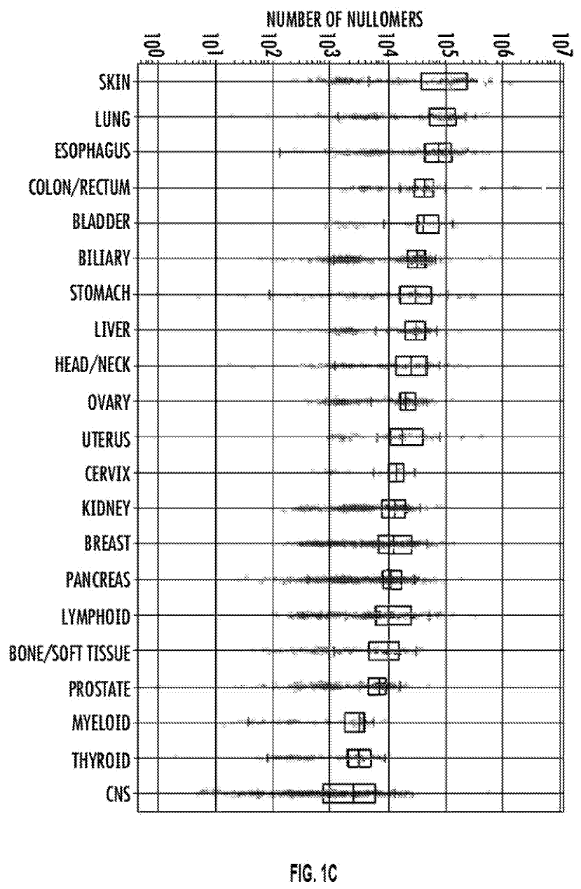

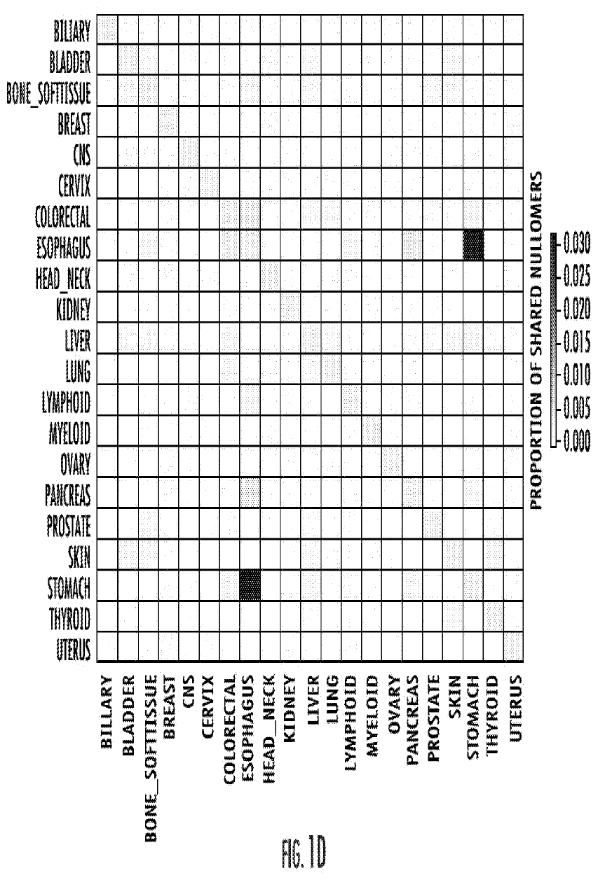

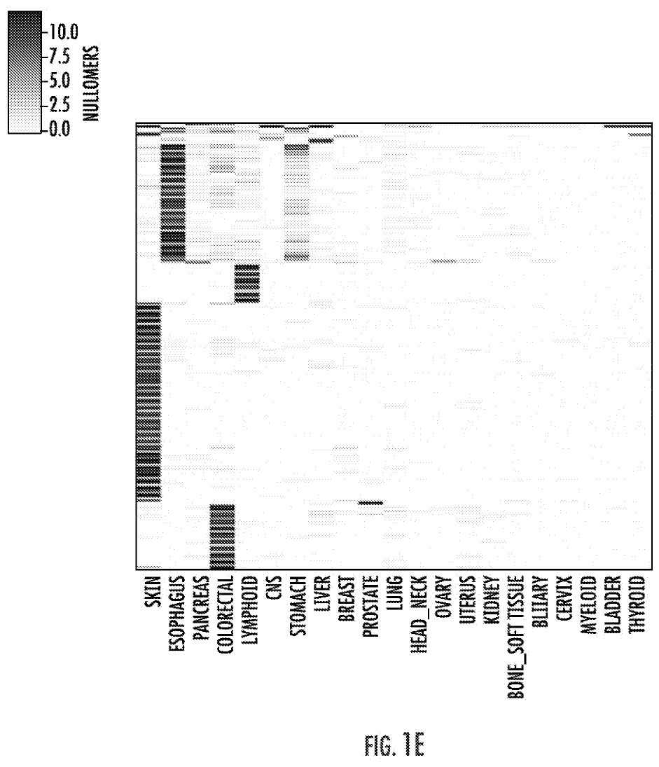

As cancer causes DNA mutations, we investigated if they can result in the resurfacing of nullomers (FIG. 1A). Using our previously characterized human nullomers (Georgakopoulos-Soares et al., 2020), we analyzed whole-genome sequencing results from 2,577 patients across 21 different cancer types from The Cancer Genome Atlas (TCGA; (ICGC/TCGA Pan-Cancer Analysis of Whole Genomes Consortium 2020)) for resurfacing nullomers. For the 44,599,472 single nucleotide substitutions we identified 186,256,551 resurfacing 16 bp nullomers across all cancer types (Table 1). Furthermore, we identified 2,470,091 nullomers resulting from short insertions and deletions (1-100 bp). The median number of nullomers created by each substitution was two and for indels four. The median number of nullomers found across cancer patients was 9,107. On average 58.29% of substitutions in a patient resulted in one or more nullomers, with only 1% of the nullomers derived from coding regions. Since we were interested in nullomers that could be used as cancer biomarkers, we focused on the subset of nullomers that are recurrent, i.e. those found in more than one patient for a specific cancer type. The number of recurrent nullomers is proportional to the total number of mutations (FIG. 1B) and the number of recurrent mutations across all patients was 1,057,787. As both the number of patients per cancer type and the mutational load vary, the median for each tissue type ranged from 0-98.

Analysis of the most frequent recurring nullomers revealed several previously known cancer-associated mutations (TABLE 4). For example, one of the most recurrent coding nullomers are the Gly12Asp, Gly12Val and Gly 12Cys missense mutations in KRAS, which is known to make up to 80% of cancer-associated KRAS mutations and causes KRAS to be constitutively active (Prior, Lewis, and Mattos, 2012; Muñoz-Maldonado, Zimmer, and Medová 2019). Although KRAS has been associated with several cancers, 190/313 (60%) of these mutations are found in pancreatic cancers. Several highly recurrent coding nullomers were also found in other known cancer-associated genes such as TP53, BRAF and PIK3CA. The top recurring nullomer mutation was located in a noncoding region, within the telomerase reverse transcriptase (TERT) promoter, which is known to be associated with numerous cancer types (Vinagre et al., 2013). This mutation, called−124C>T or C228T, is extremely common in numerous cancer types (Heidenreich et al., 2014) and thought to disrupt a G-quadruplex (Song et al., 2019) and lead to the binding of GAPB (Bell et al., 2015), an ETS transcription factor, resulting in increased TERT expression. We found this mutation in 97 patients with the highest incidence in glioblastoma (51%), fitting with its high prevalence rate and diagnostic use for this cancer type (Powter et al., 2021). There were also several nullomers that are created by different mutations (TABLE 5), e.g., CGACGTTCTGCCCACT, which was found in 74 patients at 32 loci in seven different cancers. Interestingly, some of the frequently recurrent nullomers are created by different mutations, yet they are predominantly found in one cancer. For example, GTTTTTCTCCTAGACC is found 40 times in skin cancer at 31 different loci while CTGGCAGTGAGCCACG is found 21 times in liver at 18 loci.

TABLE 4
Commonly recurring cancer-associated nullomers. Examples of frequently
recurring nullomers created by a single mutation.

| | # | | | | | # | Frequency in |
|---|---|---|---|---|---|---|---|
| Nullomer | patients | Chr:pos (hg38) | mutation | Name | Locus | cancers | cancer types |
| AGGGCCCGGA | 97 | chr5: 1295113 | G > A | -124C > T | TERT | 7 | In 20/39 of |
| AGGGGC | | | | C228T | | | GBMs, |
| | | | | | | | noncoding |
| TCTTGCCTAC | 89 | chr12: 25245350 | C > T | c.35G > A | KRAS | 6 | In 78/313 |
| GCCATC | | | | Gly12Asp | | | pancreatic |
| | | | | | | | cancers |
| CTGTTGGCGT | 79 | chr12: 25245350 | C > A | c.35G > T | KRAS | 6 | In 65/313 |
| AGGCAA | | | | Gly12Val | | | pancreatic |
| | | | | | | | cancers |
| ACGCCACGAG | 47 | chr12: 25245351 | C > G | c.34G > T | KRAS | 1 | In 47/313 |
| CTCCAA | | | | Gly12Cys | | | pancreatic |
| | | | | | | | cancers |
| GGTGCATGTT | 35 | chr17: 7673802 | C > T | c.818 G > A | TP53 | 13 | A few in |
| TGTGCC | | | | Arg273Pro | | | many cancers |
| GTGGGGGCAG | 28 | chr17: 7675088 | C > T | C524G > A | TP53 | 11 | A few in |
| TGCCTC | | | | Arg175His | | | many cancers |

TABLE 5
Frequently recurring cancer-associated
nullomers. Examples of frequently recurring
nullomers created by many different mutations

| | # | # | # | |
|---|---|---|---|---|
| Nullomer | patients | loci | cancers | Comment |
| CGACGTTC | 74 | 32 | 7 | Mainly |
| TGCCCACT | | | | pancreatic |
| | | | | and stomach |
| GTTTTTCT | 41 | 32 | 2 | All but one |
| CCTAGACC | | | | in skin |
| CTGCAGTG | 30 | 30 | 11 | Third in |
| GCGCAATA | | | | colorectal |
| TTATAGGG | 26 | 25 | 1 | Colorectal |
| GTCCAGTG | | | | only |
| CTGGCAGT | 26 | 23 | 2 | 21 in liver, |
| GAGCCACG | | | | 5 esophagus |

ii. Generation of a Cancer Subtype Nullomer Classifier

Based on the observation that most recurrent nullomers are predominantly found in one cancer type, we hypothesized that nullomers can be used to distinguish between cancer types. We filtered nullomers by keeping only those that appeared >=ri times in specific cancer type i. Comparison of the set of recurrent nullomers associated with each cancer type reveals that the overlap is small, as indicated by the Jaccard index which is <0.03, suggesting that each cancer type has a distinct nullomer signature (FIG. 1C). We also carried out an alternate analysis that counts the number of times recurrent nullomers are found for all patients, finding that patients are strongly enriched for only one set of cancer specific recurrent nullomers (FIG. 1D).

To test if these recurrent nullomers can classify tumor samples, we trained a support vector machine classifier to identify tumor type. The classifier takes as input a 21-dimensional vector indicating the number of recurrent nullomers found for each cancer specific set. Evaluation using 10-fold cross-validation, revealed that our classifier achieves both high sensitivity and specificity, with an F1 score of 0.92 and an accuracy of 0.99. The performance was better than the deep learning model recently presented by Jiao et al (Jiao et al., 2020) and also requires less computational resources to train. Moreover, the nullomer based classifier is more intuitive and easier to interpret biologically as samples are distinguished based on the number of nullomers corresponding to cancer specific sets.

iii. Nullomers can Distinguish Additional Cancer Features

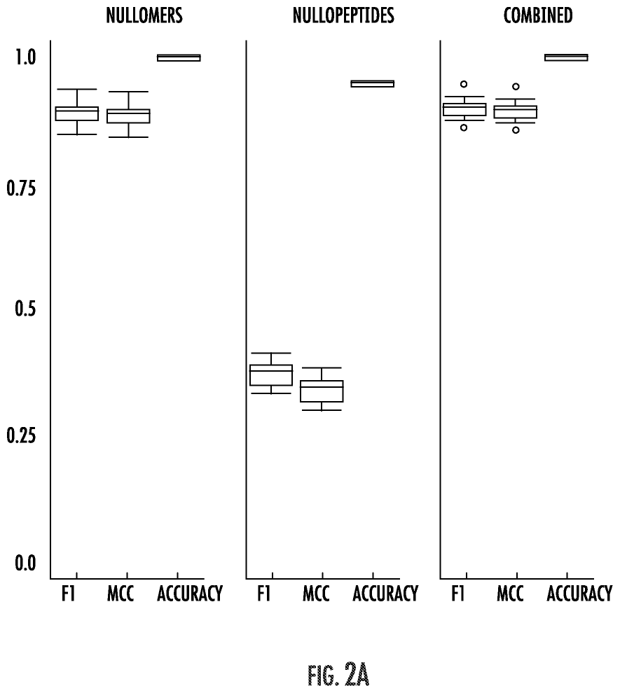

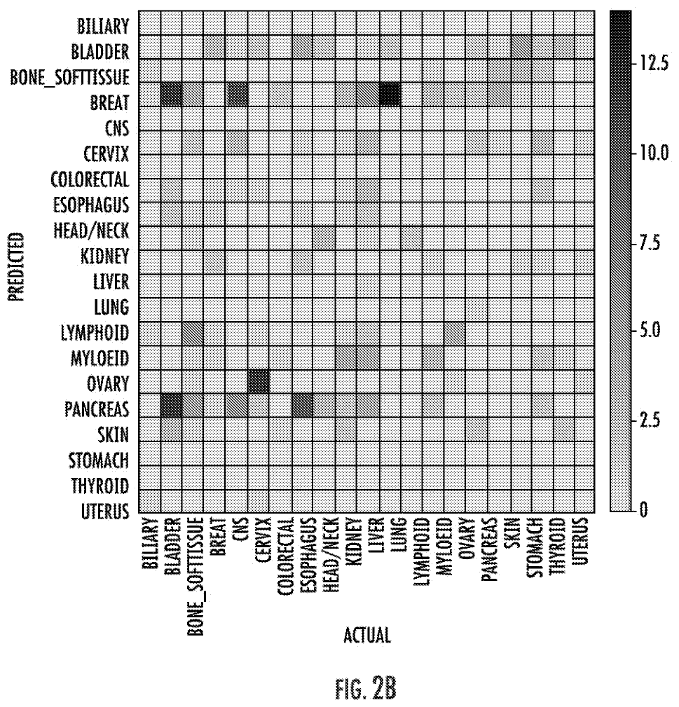

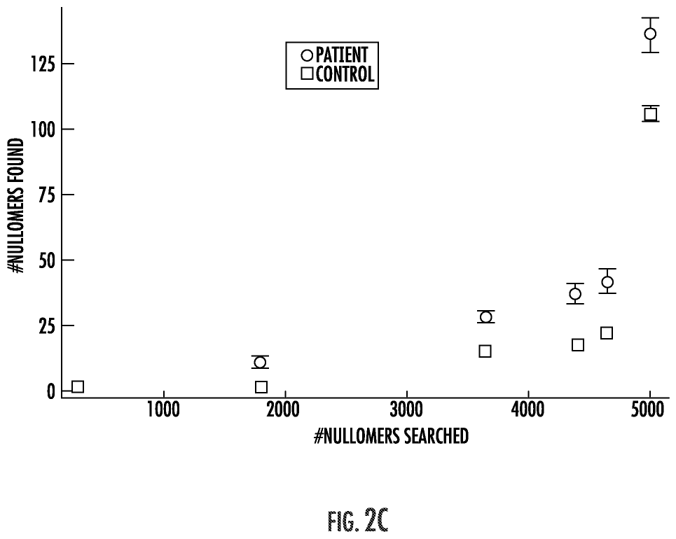

To test if nullomers could be used to distinguish other cancer features, we analyzed both breast and colorectal cancers. For breast cancer, determining whether a sample is deficient for BRCA1 or BRCA2 is important in treatment decisions (Lee, Moon, and Kim, 2020; Tung and Garber, 2018). We used a dataset of 560 breast cancers (Nik-Zainal et al., 2016), from which we selected 89 samples that were deficient for BRCA1 or BRCA2, and a similar number that were not deficient. We extracted 3,648 recurrent nullomers for the BRCA deficient samples and 1,174 for the non-BRCA deficient ones, and found that the resulting classifier achieves an accuracy of 0.76 and F1 score of 0.78 (FIG. 2C). We next analyzed colorectal cancers which can be divided into two main categories based on microsatellite stability status: Microsatellite Instable (MSI) and Microsatellite Stable (MSS). MSI is associated with a better cancer prognosis, increased benefits from surgery and sensitivity to immunotherapy but with a lack of efficacy from adjuvant treatment (Battaglin et al., 2018). We extracted recurrent nullomers for 10 MSI samples and 10 MSS samples from the breast cancer dataset. As there are 4, 120 recurring nullomers in the MSI set and only 36 in the MSS set, our classifier achieved an accuracy of 0.912.

iv. Nullomers are Enriched in ctDNA

We next tested whether nullomers could be used to diagnose cancer in cfDNA. We focused on prostate cancer, due to the following reasons: 1) our availability of both WGS datasets and cfDNA samples; 2) the number of recurrent nullomers per this subtype (N=X) is in the median range (Y) of all 21 tissues that we analyzed; and 3) the current primary screen for this cancer measures levels of the prostate-specific antigen (PSA) in the blood and has high false negative and false positive rates (Barry, 2001) and the importance of more accurate screening for minimal residual disease after treatment or surgical interventions (Cackowski and Taichman, 2018; Murray, 2018). We first excluded all common variants (p>0.05) that lead to the resurfacing of nullomers that we characterized in the human genome (Georgakopoulos-Soares et al., 2020). We then analyzed WGS from 6 cfDNA samples from prostate cancer patients and 23 controls (Ulz et al., 2019). For each nullomer that we identified in the cfDNA (both cases and controls), we characterized all possible single nucleotide substitutions in the reference genome that could give rise to this nullomer. By intersecting this list of nullomer creating substitutions with known germline variants identified by the gnomAD project (Karczewski et al., 2020), we calculated the probability that each nullomer will be present in an individual. We excluded all nullomers that are found in the population with p>0.05, leaving us with 4,665 recurrent prostate nullomers.

Another source of nullomers in cfDNA WGS could be sequencing errors. To identify nullomers that were observed due to these technical artifacts, we developed a Poisson model (see Methods). Since sequencing errors are assumed to be distributed uniformly, nullomers arising for this reason will have a profile that differs from nullomers stemming from sequences that are present in the cfDNA sample. To ensure robust detection, and to be able to compare samples of different sequencing depth, we randomly split the reads into chunks of 5× and searched for nullomers in each chunk separately. Nullomers found to be significant in at least two chunks were assumed to not be sequencing errors.

After filtering our data for both common variants and sequencing errors, we analyzed whether we observe an enrichment for nullomers in cases versus controls. We found that the mean number of recurrent prostate nullomers detected in the patient samples is 22.3, while in the healthy controls we detect 10.7. The expected number of recurrent prostate nullomers due to germline variants is 10.3, suggesting that our test is highly sensitive. Moreover, the difference between cases and controls is consistent when using more stringent cutoffs for nullomers that could emerge due to germline variants. Taken together, these results demonstrate that our prostate nullomers classifier could serve as a sensitive and specific means of identifying cancer in cfDNA samples.

To experimentally validate that nullomers could be used as a cancer diagnosis tool, we generated a custom nullomer prostate cancer probe panel for cfDNA sequence target enrichment. This panel targets 4,280 regions (385/4,665 were removed due to technical reasons) harboring loci which could result in recurrent prostate cancer associated nullomers, along with 60 bp flanking regions. We extracted cfDNA samples from 6 healthy donors and 7 prostate cancer patients of various stages. Indexed libraries were hybridized to the custom oligo pool and sequencing of the enriched libraries was done in multiplex at high coverage (×10,000-×20,000). Overall, we observed a larger number of nullomers in cases as compared to controls, similarly to our results for the WGS cfDNA. Combined, our results show that nullomers could be used as a straightforward, sensitive and specific cfDNA diagnosis tool.

v. Nullomers Alter Promoter Activity

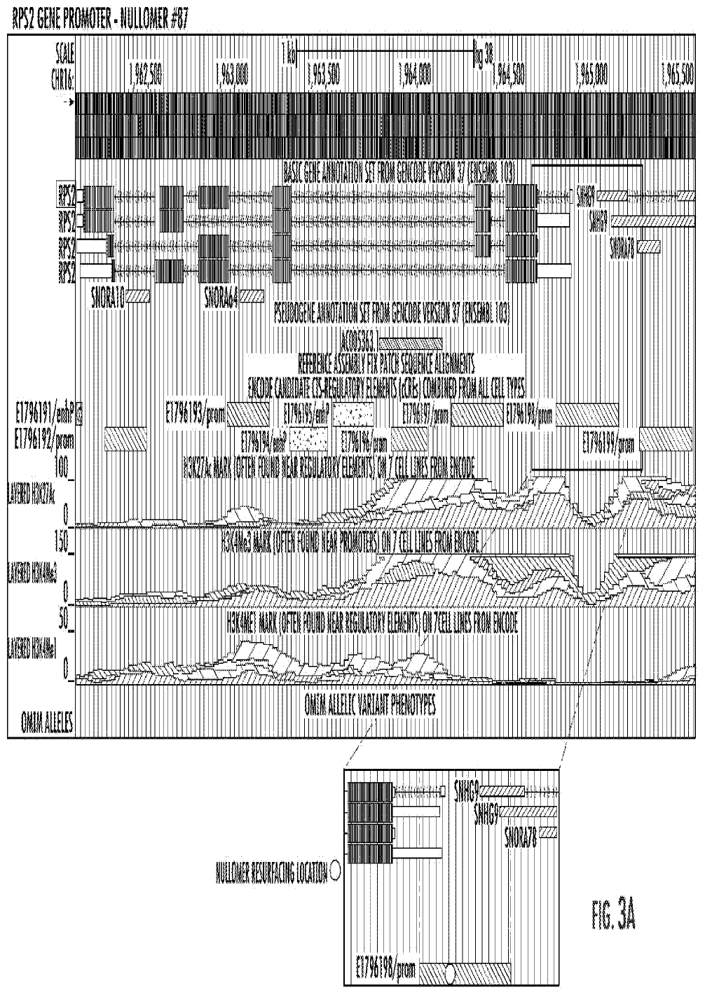

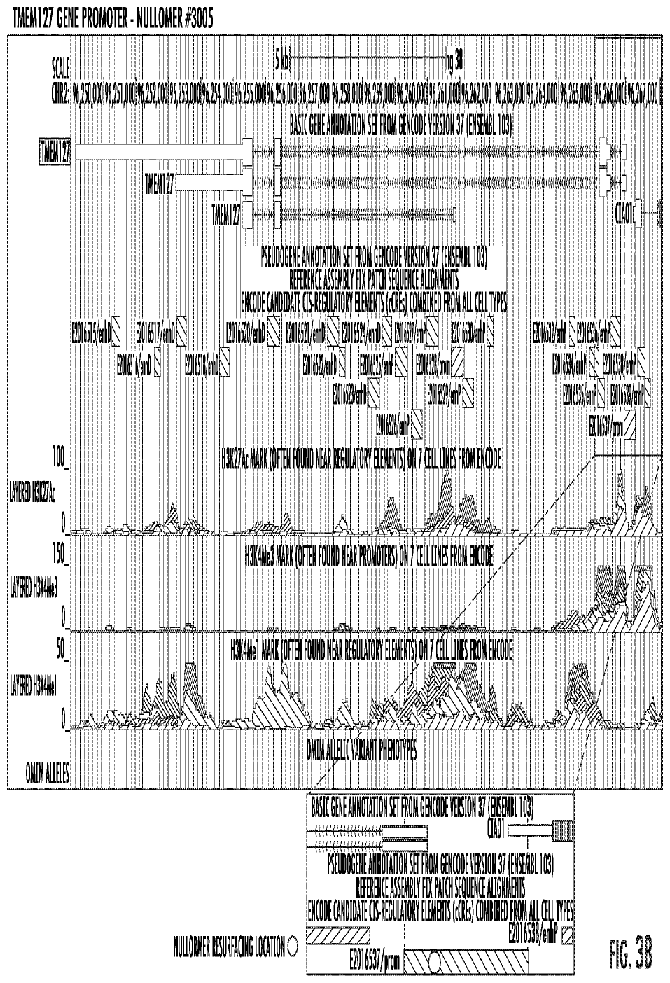

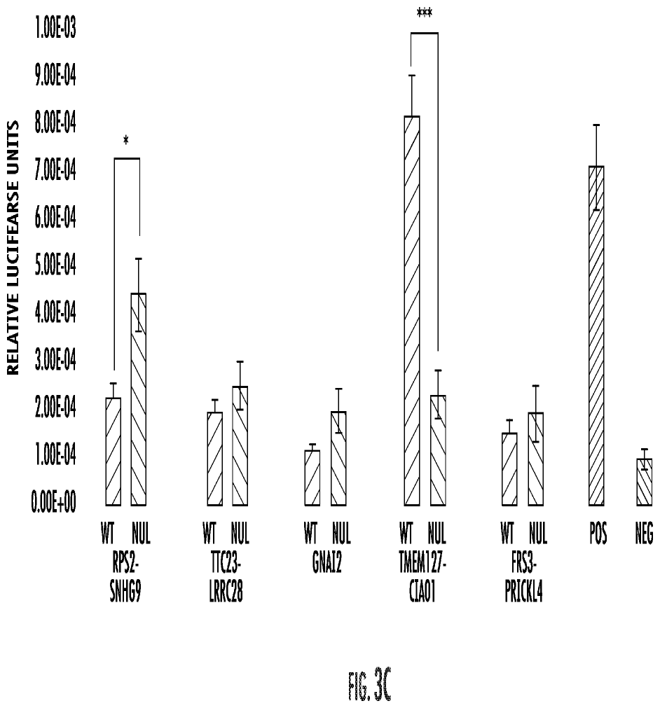

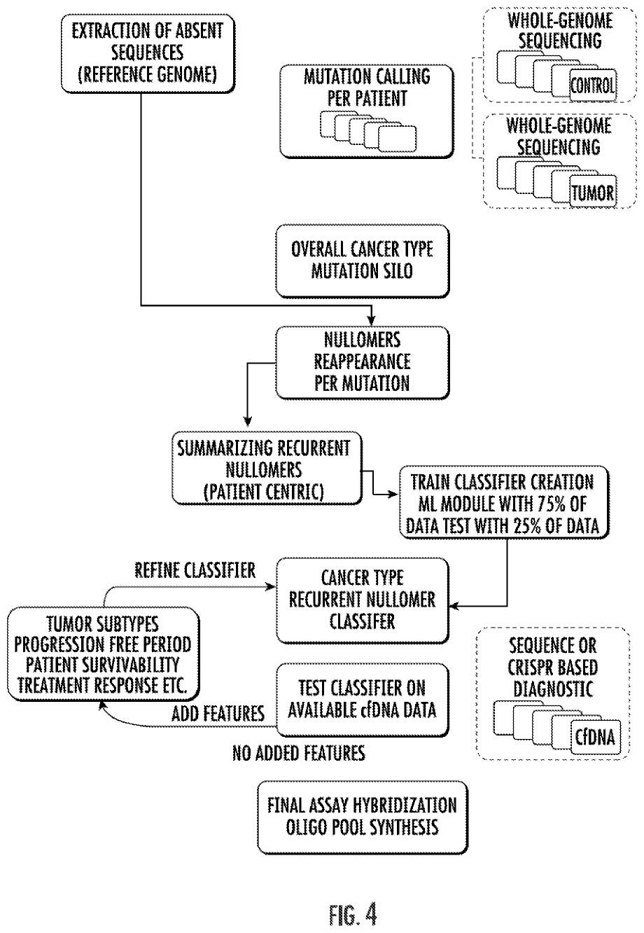

Only a small number of mutations in gene regulatory elements that affect gene expression have been found to be associated with cancer (Poulos et al., 2015). As the majority of our cancer-associated nullomers reside in noncoding sequence (99%), we tested whether nullomers could identify cancer-associated gene regulatory mutations that have a functional effect. Of note, our top recurring nullomer mutation was in the TERT promoter (TABLE 4), which is associated with numerous cancers (Vinagre et al., 2013). Focusing on prostate cancer, we selected five nullomers for luciferase reporter assays using the following criteria: i) nullomers that reside in a promoter based on ENCODE annotations (Consortium, Encode Project et al., 2012); and ii) the gene regulated by the promoter is associated with prostate cancer. Our list included nullomers in: 1) a promoter between two divergent genes, RPS2 and the lncRNA gene SNHG9, both of which are overexpressed in prostate cancer (Ohkia et al., 2004); 2) a promoter between two divergent genes, TMEM127 and CIAOI, with the former being downregulated in prostate cancer (Qin et al., 2014); 3) a promoter between two divergent genes, TT(23 and LRR(28, with the former showing aberrant splicing that relates to therapy resistance in prostate cancer cells (Bowler et al., 2018); 4) the promoter of GNA12 a protein that interacts with (′XCR5, which positively correlates with prostate cancer progression (El-Haibi et al., 2013); and 5) a promoter between two divergent genes, PRICKLE4 and FRS3, with the latter thought to affect malignant but not benign prostate cells (Valencia et al., 2011). We cloned the promoter sequence with and without the nullomer into a luciferase promoter assay vector and compared their activity in androgen-sensitive human prostate adenocarcinoma cells (LNCaP). For two out of the five assayed promoters, we observed a significant effect on reporter activity (FIG. 3). For the RPS2-SNHG9 promoter, the nullomer led to significantly increased activity in line with this gene being overexpressed in cancer (Ohkia et al., 2004). For the TMEM127-CIAOI promoter the nullomer completely abolished activity, fitting with its observed downregulation in prostate cancer (Qin et al., 2014). Combined, our experimental results show that nullomers could have a significant effect on promoter activity and could potentially be used to identify cancer associated cis-regulatory mutations.

### 3. Discussion

Cancer is a DNA mutation causing disease. Here, we show that by analysing cancer WGS datasets, we can find mutations that lead to the resurrection of nullomers. Further analyses of the recurrence of these nullomers shows that they can be used to classify not only cancer tissue origin but also additional cancer features, such as the type of breast or colorectal cancer. Analysis of cfDNA WGS datasets finds that nullomers could be used to tease out patients from control, which was further validated by testing a sequence enrichment panel on cfDNA extracted from prostate cancer patients and controls. Finally, using experimental assays, we show that nullomers have a functional effect on regulatory sequences.

Our analyses showed that in addition to tissue origin, nullomers can also be used to detect other cancer features in breast and colorectal cancer. This approach could likely be used to diagnose tumor features in other cancers types. It would also be interesting to test whether nullomers could detect additional cancer characteristics such as chance of recurrence, drug response, mortality and others. As nullomers do not exist in the human genome, they could also be great candidates for neoantigens. Previous work has shown that minimal absent words, short sequences that are absent from a genome or proteome, could be used to identify phosphorylation sites of high confidence, some of which could be associated with cancer (Koulouras and Frith, 2021). Nullomers were also shown to be effective in identifying unique peptides that are exceedingly distant from human peptides that potentially could be used as antibodies against Trypanosoma cruzi (Vergni, Gaudio, and Santoni, 2020) or SARS-COV-2 (Santoni and Vergni, 2020). Analysis of the Immune Epitope Database of validated antigens (Vita et al., 2019) found that 13 of the recurrent coding nullomers can create neoantigens with predicted strong binding levels that were subsequently validated. The expected number of neoantigens with strong binding levels is 1.72 (p-value<le-8, hypergeometric test), suggesting that missense mutations also resulting in nullomers are 7-fold more likely to also generate strongly binding neoantigens.

Nullomers could also be combined with other cancer biomarkers and risk factors to improve the diagnostic positive predictive value. For example, it was recently shown that combining a blood test that detects both protein biomarkers and DNA mutations along with positron emission tomography-computed tomography (PET-CT) could detect multiple cancers (Lennon et al., 2020). Adding specific cancer-associated coding mutations to nullomers in the screening of cfDNA could increase sensitivity and specificity. cfDNA methylation or ChIP-seq diagnostic assays could also improve this. Risk factors such as age, tobacco, alcohol, sun exposure, family history, radiation exposure, body mass index, physical activity and others could also enhance nullomer cancer diagnosis. In summary, adding nullomer-based diagnostics to existing cancer biomarkers and risk factors could improve the power to detect various cancer subtypes.

We used a sequence enrichment based assay to detect nullomers in cfDNA from blood taken from prostate cancer cases and controls. Alternate assays could potentially be used for future rapid diagnosis of cancer via nullomers. These could include the use of CRISPR-based detection tools that utilize Cas12 or Cas13 (Kellner et al., 2019). For example, recent use of Cas13 in a microwell array system allowed the rapid screening of over 4,500 targets for 169 human-associated viruses with high sensitivity and specificity (Ackerman et al., 2020). In addition, with nullomer-based diagnostics potentially not needing large amounts of cfDNA, cfDNA could be collected in a less invasive manner (blood draw), using for example urine or saliva, which were shown to be a viable but reduced source of cfDNA (Augustus et al., 2020; Ding et al., 2019).

Nullomers could be used as a novel tool to identify cancer-associated gene regulatory mutations. Amongst the 210 prostate cancer promoter nullomers, we selected five promoters and found that two of them significantly affected promoter activity due to the nullomer. Their difference in activity was in line with the gene's expression change in prostate cancer, with RPS2-SNHG9 having increased activity fitting with its overexpression in prostate cancer (Ohkia et al., 2004) and IMEM127-CIAOI abolishing activity, in line with its observed downregulation in prostate cancer (Qin et al., 2014). It is important to note that we “hand-selected” these promoters based on their prostate cancer association. Future high-throughput assays, such as massively parallel reporter assays (MPRAs; Inoue and Ahituv, 2015) that can test thousands of sequences and variants for their regulatory activity, could be used to test the effect of nullomers on gene regulation in an unbiased manner.

Our analyses used 2,577 patients with 21 different common cancer types to develop a cancer tissue of origin classifier. Additional genomes from tumor tissues, controls and cfDNA could improve this classifier even more. For rare cancer types, obtaining WGS datasets from tumor, matched control and cfDNA would be extremely helpful in allowing our nullomer classifier to detect these cancer types. It would also be interesting to assess how our nullomer classifier functions in cancers with a lower amount of mutations.

In summary, we show that nullomers can provide a powerful tool for cancer diagnosis. As they can easily be detected via sequence or CRISPR-based tools, it should be straightforward to integrate them in current routine cancer diagnostic tests and their use could increase the sensitivity and specificity of these tests. Combining nullomer-based screening with clinical characteristics and additional diagnostic tools/features could increase the positive predictive value of this diagnostic. In addition, as cfDNA could also be isolated from urine and saliva and detection of these sequences does not need a large amount of DNA, nullomer-based diagnosis could be carried out in a non-invasive manner. Our work also suggests that nullomers could be used to highlight cancer-associated gene regulatory mutations which have been difficult to identify. Further high-throughput characterization of these mutations could allow the detection of bona fide cancer-associated functional regulatory mutations that could be used for diagnosis and treatment.

### Example 2. Detection of Nullomers by CRISPR Diagnosis

The presence, absence or quantity of one or a plurality of the nullomers disclosed herein can be detected by CRISPR diagnosis. To this end, non-limiting exemplary sgRNA sequences that can be used to detect nullomers in cancer are provided in TABLE 6. As shown in TABLE 6, some of the nullomers are recurrent in several cancers (see NULLOMER INFO). Depending on the Cas protein used, different recognition pattern for sgRNAs is required. TABLE 6 exemplifies sgRNAs fitting either the Cas9 (saCas9) or the Cas12 (AsCpf1/LbCpf1 RR) protein.

TABLE 6
Non-limiting exemplary sgRNA sequences for nullomer detection in cancer.
NULLOMER INFO

| #null- | Se- | nmuta- | | | | refer- | muta- | cancers |
|---|---|---|---|---|---|---|---|---|
| omer | quence | tions | nloci | chr | pos | ence | tion | |
| 1 | gcccc | 97 | 1 | chr5 | 1295113 | G | A | Biliary(1), Bone_SoftTissue(2), Cervix(1), |
| | ttccg | | | | | | | Lymphoid(1), Bladder(11), Thyroid(11), |
| | ggccc | | | | | | | Head_neck(3), Kidney(2), Lung(1), |
| | t | | | | | | | CNS(35), Colorectal(1), Skin(16), Liver(12) |
| 2 | gcccc | 97 | 1 | chr5 | 1295113 | G | A | Biliary(1), Bone_SoftTissue(2), Cervix(1), |
| | ttccg | | | | | | | Lymphoid(1), Bladder(11), Thyroid(11), |
| | ggccc | | | | | | | Head_neck(3), Kidney(2), Lung(1), |
| | t | | | | | | | CNS(35), Colorectal(1), Skin(16), Liver(12) |
| 3 | gcccc | 97 | 1 | chr5 | 1295113 | G | A | Biliary(1), Bone_SoftTissue(2), Cervix(1), |
| | ttccg | | | | | | | Lymphoid(1), Bladder(11), Thyroid(11), |
| | ggccc | | | | | | | Head_neck(3), Kidney(2), Lung(1), |
| | t | | | | | | | CNS(35), Colorectal(1), Skin(16), Liver(12) |
| 4 | gcccc | 97 | 1 | chr5 | 1295113 | G | A | Biliary(1), Bone_SoftTissue(2), Cervix(1), |
| | ttccg | | | | | | | Lymphoid(1), Bladder(11), Thyroid(11), |
| | ggccc | | | | | | | Head_neck(3), Kidney(2), Lung(1), |
| | t | | | | | | | CNS(35), Colorectal(1), Skin(16), Liver(12) |
| 5 | gcccc | 97 | 1 | chr5 | 1295113 | G | A | Biliary(1), Bone_SoftTissue(2), Cervix(1), |
| | ttccg | | | | | | | Lymphoid(1), Bladder(11), Thyroid(11), |
| | ggccc | | | | | | | Head_neck(3), Kidney(2), Lung(1), |
| | t | | | | | | | |
| | | | | | | | | CNS(35), Colorectal(1), Skin(16), Liver(12) |
| 6 | gcccc | 97 | 1 | chr5 | 1295113 | G | A | Biliary(1), Bone_SoftTissue(2), Cervix(1), |
| | ttccg | | | | | | | Lymphoid(1), Bladder(11), Thyroid(11), |
| | ggccc | | | | | | | Head_neck(3), Kidney(2), Lung(1), |
| | t | | | | | | | CNS(35), Colorectal(1), Skin(16), Liver(12) |
| 7 | ccctt | 97 | 1 | chr5 | 1295113 | G | A | Biliary(1), Bone_SoftTissue(2), Cervix(1), |
| | ccggg | | | | | | | Lymphoid(1), Bladder(11), Thyroid(11), |
| | ccctc | | | | | | | Head_neck(3), Kidney(2), Lung(1), |
| | c | | | | | | | CNS(35), Colorectal(1), Skin(16), Liver(12) |
| 8 | ccctt | 97 | 1 | chr5 | 1295113 | G | A | Biliary(1), Bone_SoftTissue(2), Cervix(1), |
| | ccggg | | | | | | | Lymphoid(1), Bladder(11), Thyroid(11), |
| | ccctc | | | | | | | Head_neck(3), Kidney(2), Lung(1), |
| | c | | | | | | | CNS(35), Colorectal(1), Skin(16), Liver(12) |
| 9 | ccctt | 97 | 1 | chr5 | 1295113 | G | A | Biliary(1), Bone_SoftTissue(2), Cervix(1), |
| | ccggg | | | | | | | Lymphoid(1), Bladder(11), Thyroid(11), |
| | ccctc | | | | | | | Head neck(3), Kidney(2), Lung(1), |
| | c | | | | | | | CNS(35), Colorectal(1), Skin(16), Liver(12) |
| 10 | ccctt | 97 | 1 | chr5 | 1295113 | G | A | Biliary(1), Bone_SoftTissue(2), Cervix(1), |
| | ccggg | | | | | | | Lymphoid(1), Bladder(11), Thyroid(11), |
| | ccctc | | | | | | | Head_neck(3), Kidney(2), Lung(1), |
| | c | | | | | | | CNS(35), Colorectal(1), Skin(16), Liver(12) |
| 11 | ccctt | 97 | 1 | chr5 | 1295113 | G | A | Biliary(1), Bone_SoftTissue(2), Cervix(1), |
| | ccggg | | | | | | | Lymphoid(1), Bladder(11), Thyroid(11), |
| | ccctc | | | | | | | Head neck(3), Kidney(2), Lung(1), |
| | c | | | | | | | CNS(35), Colorectal(1), Skin(16), Liver(12) |
| 12 | ccctt | 97 | 1 | chr5 | 1295113 | G | A | Biliary(1), Bone_SoftTissue(2), Cervix(1), |
| | ccggg | | | | | | | Lymphoid(1), Bladder(11), Thyroid(11), |
| | ccctc | | | | | | | Head_neck(3), Kidney(2), Lung(1), |
| | c | | | | | | | CNS(35), Colorectal(1), Skin(16), Liver(12) |
| 13 | agggc | 97 | 1 | chr5 | 1295113 | G | A | Biliary(1), Bone_SoftTissue(2), Cervix(1), |
| | ccgga | | | | | | | Lymphoid(1), Bladder(11), Thyroid(11), |
| | agggg | | | | | | | Head_neck(3), Kidney(2), Lung(1), |
| | c | | | | | | | CNS(35), Colorectal(1), Skin(16), Liver(12) |
| 14 | agggc | 97 | 1 | chr5 | 1295113 | G | A | Biliary(1), Bone_SoftTissue(2), Cervix(1), |
| | ccgga | | | | | | | Lymphoid(1), Bladder(11), Thyroid(11), |
| | agggg | | | | | | | Head_neck(3), Kidney(2), Lung(1), |
| | c | | | | | | | CNS(35), Colorectal(1), Skin(16), Liver(12) |
| 15 | agggc | 97 | 1 | chr5 | 1295113 | G | A | Biliary(1), Bone_SoftTissue(2), Cervix(1), |
| | ccgga | | | | | | | Lymphoid(1), Bladder(11), Thyroid(11), |
| | agggg | | | | | | | Head_neck(3), Kidney(2), Lung(1), |
| | c | | | | | | | CNS(35), Colorectal(1), Skin(16), Liver(12) |
| 16 | agggc | 97 | 1 | chr5 | 1295113 | G | A | Biliary(1), Bone_SoftTissue(2), Cervix(1), |
| | ccgga | | | | | | | Lymphoid(1), Bladder(11), Thyroid(11), |
| | agggg | | | | | | | Head_neck(3), Kidney(2), Lung(1), |
| | c | | | | | | | CNS(35), Colorectal(1), Skin(16), Liver(12) |
| 17 | agggc | 97 | 1 | chr5 | 1295113 | G | A | Biliary(1), Bone_SoftTissue(2), Cervix(1), |
| | ccgga | | | | | | | Lymphoid(1), Bladder(11), Thyroid(11), |
| | agggg | | | | | | | Head_neck(3), Kidney(2), Lung(1), |
| | c | | | | | | | CNS(35), Colorectal(1), Skin(16), Liver(12) |
| 18 | agggc | 97 | 1 | chr5 | 1295113 | G | A | Biliary(1), Bone_SoftTissue(2), Cervix(1), |
| | ccgga | | | | | | | Lymphoid(1), Bladder(11), Thyroid(11), |
| | agggg | | | | | | | Head neck(3), Kidney(2), Lung(1), |
| | c | | | | | | | CNS(35), Colorectal(1), Skin(16), Liver(12) |
| 19 | tcttg | 89 | 1 | chr12 | 25245350 | C | T | Lung(1), Biliary(1), Pancreas(78), Cervix(1), |
| | cctac | | | | | | | Esophagus(2), Colorectal(4), Head_neck(1), |
| | gccat | | | | | | | Skin(1) |
| | c | | | | | | | |
| 20 | tcttg | 89 | 1 | chr12 | 25245350 | C | T | Lung(1), Biliary(1), Pancreas(78), Cervix(1), |
| | cctac | | | | | | | Esophagus(2), Colorectal(4), Head_neck(1), |
| | gccat | | | | | | | Skin(1) |
| | c | | | | | | | |
| 21 | tcttg | 89 | 1 | chr12 | 25245350 | C | T | Lung(1), Biliary(1), Pancreas(78), Cervix(1), |
| | cctac | | | | | | | Esophagus(2), Colorectal(4), Head_neck(1), |
| | gccat | | | | | | | Skin(1) |
| | c | | | | | | | |
| 22 | gatgg | 89 | 1 | chr12 | 25245350 | C | T | Lung(1), Biliary(1), Pancreas(78), Cervix(1), |
| | cgtag | | | | | | | Esophagus(2), Colorectal(4), Head_neck(1), |
| | gcaag | | | | | | | Skin(1) |
| | a | | | | | | | |
| 23 | gatgg | 89 | 1 | chr12 | 25245350 | C | T | Lung(1), Biliary(1), Pancreas(78), Cervix(1), |
| | cgtag | | | | | | | Esophagus(2), Colorectal(4), Head_neck(1), |
| | gcaag | | | | | | | Skin(1) |
| | a | | | | | | | |
| 24 | gatgg | 89 | 1 | chr12 | 25245350 | C | T | Lung(1), Biliary(1), Pancreas(78), Cervix(1), |
| | cgtag | | | | | | | Esophagus(2), Colorectal(4), Head_neck(1), |
| | gcaag | | | | | | | Skin(1) |
| | a | | | | | | | |
| 25 | ctgtt | 79 | 1 | chr12 | 25245350 | C | A | Lung(3), Liver(1), Uterus(1), Pancreas(65), |
| | ggcgt | | | | | | | Cervix(1), Ovary(1), Colorectal(7) |
| | aggca | | | | | | | |
| | a | | | | | | | |
| 26 | ctgtt | 79 | 1 | chr12 | 25245350 | C | A | Lung(3), Liver(1), Uterus(1), Pancreas(65), |
| | ggcgt | | | | | | | Cervix(1), Ovary(1), Colorectal(7) |
| | aggca | | | | | | | |
| | a | | | | | | | |
| 27 | ctgtt | 79 | 1 | chr12 | 25245350 | C | A | Lung(3), Liver(1), Uterus(1), Pancreas(65), |
| | ggcgt | | | | | | | Cervix(1), Ovary(1), Colorectal(7) |
| | aggca | | | | | | | |
| | a | | | | | | | |
| 28 | gccaa | 79 | 1 | chr12 | 25245350 | C | A | Lung(3), Liver(1), Uterus(1), Pancreas(65), |
| | cagct | | | | | | | Cervix(1), Ovary(1), Colorectal(7) |
| | ccaac | | | | | | | |
| | t | | | | | | | |
| 29 | gccaa | 79 | 1 | chr12 | 25245350 | C | A | Lung(3), Liver(1), Uterus(1), Pancreas(65), |
| | cagct | | | | | | | Cervix(1), Ovary(1), Colorectal(7) |
| | ccaac | | | | | | | |
| | t | | | | | | | |
| 30 | gccaa | 79 | 1 | chr12 | 25245350 | C | A | Lung(3), Liver(1), Uterus(1), Pancreas(65), |
| | cagct | | | | | | | Cervix(1), Ovary(1), Colorectal(7) |
| | ccaac | | | | | | | |
| | t | | | | | | | |
| 31 | ttgcc | 79 | 1 | chr12 | 25245350 | C | A | Lung(3), Liver(1), Uterus(1), Pancreas(65), |
| | tacgc | | | | | | | Cervix(1), Ovary(1), Colorectal(7) |
| | caaca | | | | | | | |
| | g | | | | | | | |
| 32 | ttgcc | 79 | 1 | chr12 | 25245350 | C | A | Lung(3), Liver(1), Uterus(1), Pancreas(65), |
| | tacgc | | | | | | | Cervix(1), Ovary(1), Colorectal(7) |
| | caaca | | | | | | | |
| | g | | | | | | | |
| 33 | ttgcc | 79 | 1 | chr12 | 25245350 | C | A | Lung(3), Liver(1), Uterus(1), Pancreas(65), |
| | tacgc | | | | | | | Cervix(1), Ovary(1), Colorectal(7) |
| | caaca | | | | | | | |
| | g | | | | | | | |
| 34 | agttg | 79 | 1 | chr12 | 25245350 | C | A | Lung(3), Liver(1), Uterus(1), Pancreas(65), |
| | gagct | | | | | | | Cervix(1), Ovary(1), Colorectal(7) |
| | gttgg | | | | | | | |
| | c | | | | | | | |
| 35 | agttg | 79 | 1 | chr12 | 25245350 | C | A | Lung(3), Liver(1), Uterus(1), Pancreas(65), |
| | gagct | | | | | | | Cervix(1), Ovary(1), Colorectal(7) |
| | gttgg | | | | | | | |
| | c | | | | | | | |
| 36 | agttg | 79 | 1 | chr12 | 25245350 | C | A | Lung(3), Liver(1), Uterus(1), Pancreas(65), |
| | gagct | | | | | | | Cervix(1), Ovary(1), Colorectal(7) |
| | gttgg | | | | | | | |
| | c | | | | | | | |
| 37 | tcgag | 60 | 1 | chr7 | 1.41E+08 | A | T | CNS(3), Lymphoid(1), Thyroid(10), |
| | atttc | | | | | | | Colorectal(1), Skin(45) |
| | tctgt | | | | | | | |
| | a | | | | | | | |
| 38 | tcgag | 60 | 1 | chr7 | 1.41E+08 | A | T | CNS(3), Lymphoid(1), Thyroid(10), |
| | atttc | | | | | | | Colorectal(1), Skin(45) |
| | tctgt | | | | | | | |
| | a | | | | | | | |
| 39 | tacag | 60 | 1 | chr7 | 1.41E+08 | A | T | CNS(3), Lymphoid(1), Thyroid(10), |
| | agaaa | | | | | | | Colorectal(1), Skin(45) |
| | tctcg | | | | | | | |
| | a | | | | | | | |
| 40 | tacag | 60 | 1 | chr7 | 1.41E+08 | A | T | CNS(3), Lymphoid(1), Thyroid(10), |
| | agaaa | | | | | | | Colorectal(1), Skin(45) |
| | tctcg | | | | | | | |
| | a | | | | | | | |
| 41 | gtaaa | 14 | 9 | chr1 | 70905567 | A | C | Pancreas(1), Stomach(1), Esophagus(10), |
| | gctcc | | | | | | | Colorectal(1), Skin(1) |
| | aaagt | | | | | | | |
| | g | | | | | | | |
| 42 | gtaaa | 14 | 9 | chr1 | 70905567 | A | C | Pancreas(1), Stomach(1), Esophagus(10), |
| | gctcc | | | | | | | Colorectal(1), Skin(1) |
| | aaagt | | | | | | | |

g

SaCas9 (PAM = NNGRR) sgRNA

| #null- | Posi- | | Se- | | Specificity | Efficiency |
|---|---|---|---|---|---|---|
| omer | tion | Strand | quence | PAM | Score | Score |
| 1 | 1295110 | 1 | agggg | ggggg | 66.63373 | 5.275862 |
| | | | ctggg | | | |
| | | | agggc | | | |
| | | | ccgga | | | |
| 2 | 1295115 | 1 | ctggg | ctggg | 64.962 | 0.576663 |
| | | | agggc | | | |
| | | | ccgga | | | |
| | | | ggggg | | | |
| 3 | 1295115 | −1 | ggtcc | ccggg | 73.77797 | 3.389292 |
| | | | ccggc | | | |
| | | | ccagc | | | |
| | | | cccct | | | |
| 4 | 1295120 | 1 | agggc | ccggg | 46.16359 | 0.973002 |
| | | | ccgga | | | |
| | | | ggggg | | | |
| | | | ctggg | | | |
| 5 | 1295121 | 1 | gggcc | cgggg | 59.12599 | 1.296501 |
| | | | cggag | | | |
| | | | ggggc | | | |
| | | | tgggc | | | |
| 6 | 1295122 | 1 | ggccc | gggga | 66.75272 | 0.392485 |
| | | | ggagg | | | |
| | | | gggct | | | |
| | | | gggcc | | | |
| 7 | 1295110 | 1 | agggg | ggggg | 66.63373 | 5.275862 |
| | | | ctggg | | | |
| | | | agggc | | | |
| | | | ccgga | | | |
| 8 | 1295115 | 1 | ctggg | ctggg | 64.962 | 0.576663 |
| | | | agggc | | | |
| | | | ccgga | | | |
| | | | ggggg | | | |
| 9 | 1295115 | −1 | ggtcc | ccggg | 73.77797 | 3.389292 |
| | | | ccggc | | | |
| | | | ccagc | | | |
| | | | cccct | | | |
| 10 | 1295120 | 1 | agggc | ccggg | 46.16359 | 0.973002 |
| | | | ccgga | | | |
| | | | ggggg | | | |
| | | | ctggg | | | |
| 11 | 1295121 | 1 | gggcc | cgggg | 59.12599 | 1.296501 |
| | | | cggag | | | |
| | | | ggggc | | | |
| | | | tgggc | | | |
| 12 | 1295122 | 1 | ggccc | gggga | 66.75272 | 0.392485 |
| | | | ggagg | | | |
| | | | gggct | | | |
| | | | gggcc | | | |
| 13 | 1295110 | 1 | agggg | ggggg | 66.63373 | 5.275862 |
| | | | ctggg | | | |
| | | | agggc | | | |
| | | | ccgga | | | |
| 14 | 1295115 | 1 | ctggg | ctggg | 64.962 | 0.576663 |
| | | | agggc | | | |
| | | | ccgga | | | |
| | | | ggggg | | | |
| 15 | 1295115 | −1 | ggtcc | ccggg | 73.77797 | 3.389292 |
| | | | ccggc | | | |
| | | | ccagc | | | |
| | | | cccct | | | |
| 16 | 1295120 | 1 | agggc | ccggg | 46.16359 | 0.973002 |
| | | | ccgga | | | |
| | | | ggggg | | | |
| | | | ctggg | | | |
| 17 | 1295121 | 1 | gggcc | cgggg | 59.12599 | 1.296501 |
| | | | cggag | | | |
| | | | ggggc | | | |
| | | | tgggc | | | |
| 18 | 1295122 | 1 | ggccc | gggga | 66.75272 | 0.392485 |
| | | | ggagg | | | |
| | | | gggct | | | |
| | | | gggcc | | | |
| 19 | 25245343 | −1 | ttgga | aagag | 79.84923 | 4.862157 |
| | | | gctgg | | | |
| | | | tggcg | | | |
| | | | taggc | | | |
| 20 | 25245362 | −1 | gaata | tggag | 58.54506 | 2.813947 |
| | | | taaac | | | |
| | | | ttgtg | | | |
| | | | gtagt | | | |
| 21 | 25245363 | −1 | tgaat | ttgga | 56.99582 | 1.855413 |
| | | | ataaa | | | |
| | | | cttgt | | | |
| | | | ggtag | | | |
| 22 | 25245343 | −1 | ttgga | aagag | 79.84923 | 4.862157 |
| | | | gctgg | | | |
| | | | tggcg | | | |
| | | | taggc | | | |
| 23 | 25245362 | −1 | gaata | tggag | 58.54506 | 2.813947 |
| | | | taaac | | | |
| | | | ttgtg | | | |
| | | | gtagt | | | |
| 24 | 25245363 | −1 | tgaat | ttgga | 56.99582 | 1.855413 |
| | | | ataaa | | | |
| | | | cttgt | | | |
| | | | ggtag | | | |
| 25 | 25245343 | −1 | ttgga | aagag | 79.84923 | 4.862157 |
| | | | gctgg | | | |
| | | | tggcg | | | |
| | | | taggc | | | |
| 26 | 25245362 | −1 | gaata | tggag | 58.54506 | 2.813947 |
| | | | taaac | | | |
| | | | ttgtg | | | |
| | | | gtagt | | | |
| 27 | 25245363 | −1 | tgaat | ttgga | 56.99582 | 1.855413 |
| | | | ataaa | | | |
| | | | cttgt | | | |
| | | | ggtag | | | |
| 28 | 25245343 | −1 | ttgga | aagag | 79.84923 | 4.862157 |
| | | | gctgg | | | |
| | | | tggcg | | | |
| | | | taggc | | | |
| 29 | 25245362 | −1 | gaata | tggag | 58.54506 | 2.813947 |
| | | | taaac | | | |
| | | | ttgtg | | | |
| | | | gtagt | | | |
| 30 | 25245363 | −1 | tgaat | ttgga | 56.99582 | 1.855413 |
| | | | ataaa | | | |
| | | | cttgt | | | |
| | | | ggtag | | | |
| 31 | 25245343 | −1 | ttgga | aagag | 79.84923 | 4.862157 |
| | | | gctgg | | | |
| | | | tggcg | | | |
| | | | taggc | | | |
| 32 | 25245362 | −1 | gaata | tggag | 58.54506 | 2.813947 |
| | | | taaac | | | |
| | | | ttgtg | | | |
| | | | gtagt | | | |
| 33 | 25245363 | −1 | tgaat | ttgga | 56.99582 | 1.855413 |
| | | | ataaa | | | |
| | | | cttgt | | | |
| | | | ggtag | | | |
| 34 | 25245343 | −1 | ttgga | aagag | 79.84923 | 4.862157 |
| | | | gctgg | | | |
| | | | tggcg | | | |
| | | | taggc | | | |
| 35 | 25245362 | −1 | gaata | tggag | 58.54506 | 2.813947 |
| | | | taaac | | | |
| | | | ttgtg | | | |
| | | | gtagt | | | |
| 36 | 25245363 | −1 | tgaat | ttgga | 56.99582 | 1.855413 |
| | | | ataaa | | | |
| | | | cttgt | | | |
| | | | ggtag | | | |
| 37 | 1.41E+08 | −1 | ctagc | atgga | 90.49283 | 53.53344 |
| | | | tacag | | | |
| | | | tgaaa | | | |
| | | | tctcg | | | |
| 38 | 1.41E+08 | −1 | gtgat | gtgaa | 62.68022 | 45.66009 |
| | | | tttgg | | | |
| | | | tctag | | | |
| | | | ctaca | | | |
| 39 | 1.41E+08 | −1 | ctagc | atgga | 90.49283 | 53.53344 |
| | | | tacag | | | |
| | | | tgaaa | | | |
| | | | tctcg | | | |
| 40 | 1.41E+08 | −1 | gtgat | gtgaa | 62.68022 | 45.66009 |
| | | | tttgg | | | |
| | | | tctag | | | |
| | | | ctaca | | | |
| 41 | 70905553 | 1 | tggag | ttgga | 88.12191 | 16.55866 |
| | | | tcaaa | | | |
| | | | ggata | | | |
| | | | tcact | | | |
| 42 | 70905554 | 1 | ggagt | tggag | 83.46563 | 30.50862 |
| | | | caaag | | | |
| | | | gatat | | | |
| | | | cactt | | | |

AsCpf1/LbCpf1 RR (PAM = TYCV) sgRNA

| #null- | | | Se- | | Specificity |
|---|---|---|---|---|---|
| omer | Position | Strand | quence | PAM | Score |
| 1 | 1295090 | −1 | ggccc | tccg | 67.70082 |
| | | | tccca | | |
| | | | gcccc | | |
| | | | tcccc | | |
| 2 | 1295107 | −1 | cggcc | tccc | 86.39843 |
| | | | cagcc | | |
| | | | ccctc | | |
| | | | cgggc | | |
| 3 | NA | NA | NA | NA | NA |
| 4 | NA | NA | NA | NA | NA |
| 5 | NA | NA | NA | NA | NA |
| 6 | NA | NA | NA | NA | NA |
| 7 | 1295090 | −1 | ggccc | tccg | 67.70082 |
| | | | tccca | | |
| | | | gcccc | | |
| | | | tcccc | | |
| 8 | 1295107 | −1 | cggcc | tccc | 86.39843 |
| | | | cagcc | | |
| | | | ccctc | | |
| | | | cgggc | | |
| 9 | NA | NA | NA | NA | NA |
| 10 | NA | NA | NA | NA | NA |
| 11 | NA | NA | NA | NA | NA |
| 12 | NA | NA | NA | NA | NA |
| 13 | 1295090 | −1 | ggccc | tccg | 67.70082 |
| | | | tccca | | |
| | | | gcccc | | |
| | | | tcccc | | |
| 14 | 1295107 | −1 | cggcc | tccc | 86.39843 |
| | | | cagcc | | |
| | | | ccctc | | |
| | | | cgggc | | |
| 15 | NA | NA | NA | NA | NA |
| 16 | NA | NA | NA | NA | NA |
| 17 | NA | NA | NA | NA | NA |
| 18 | NA | NA | NA | NA | NA |
| 19 | 25245345 | 1 | gtcaa | tatc | 97.57351 |
| | | | ggcac | | |
| | | | tcttg | | |
| | | | cctac | | |
| 20 | 25245353 | −1 | aactt | tata | 65.59183 |
| | | | gtggt | | |
| | | | agttg | | |
| | | | gagct | | |
| 21 | NA | NA | NA | NA | NA |
| 22 | 25245345 | 1 | gtcaa | tatc | 97.57351 |
| | | | ggcac | | |
| | | | tcttg | | |
| | | | cctac | | |
| 23 | 25245353 | −1 | aactt | tata | 65.59183 |
| | | | gtggt | | |
| | | | agttg | | |
| | | | gagct | | |
| 24 | NA | NA | NA | NA | NA |
| 25 | 25245345 | 1 | gtcaa | tatc | 97.57351 |
| | | | ggcac | | |
| | | | tcttg | | |
| | | | cctac | | |
| 26 | 25245353 | −1 | aactt | tata | 65.59183 |
| | | | gtggt | | |
| | | | agttg | | |
| | | | gagct | | |
| 27 | NA | NA | NA | NA | NA |
| 28 | 25245345 | 1 | gtcaa | tatc | 97.57351 |
| | | | ggcac | | |
| | | | tcttg | | |
| | | | cctac | | |
| 29 | 25245353 | −1 | aactt | tata | 65.59183 |
| | | | gtggt | | |
| | | | agttg | | |
| | | | gagct | | |
| 30 | NA | NA | NA | NA | NA |
| 31 | 25245345 | 1 | gtcaa | tatc | 97.57351 |
| | | | ggcac | | |
| | | | tcttg | | |
| | | | cctac | | |
| 32 | 25245353 | −1 | aactt | tata | 65.59183 |
| | | | gtggt | | |
| | | | agttg | | |
| | | | gagct | | |
| 33 | NA | NA | NA | NA | NA |
| 34 | 25245345 | 1 | gtcaa | tatc | 97.57351 |
| | | | ggcac | | |
| | | | tcttg | | |
| | | | cctac | | |
| 35 | 25245353 | −1 | aactt | tata | 65.59183 |
| | | | gtggt | | |
| | | | agttg | | |
| | | | gagct | | |
| 36 | NA | NA | NA | NA | NA |
| 37 | 1.41E+08 | 1 | ctgta | ttca | 64.98878 |
| | | | gctag | | |
| | | | accaa | | |
| | | | aatca | | |
| 38 | NA | NA | NA | NA | NA |
| 39 | 1.41E+08 | 1 | ctgta | ttca | 64.98878 |
| | | | gctag | | |
| | | | accaa | | |
| | | | aatca | | |
| 40 | NA | NA | NA | NA | NA |
| 41 | 70905538 | −1 | aagtg | tcca | 94.80388 |
| | | | atatc | | |
| | | | ctttg | | |
| | | | actcc | | |
| 42 | 70905565 | −1 | gcagg | tcca | 97.30514 |
| | | | gcagt | | |
| | | | caaat | | |
| | | | cttaa | | |

### Example 3. Detection of Cancer Subtype by Nullomers

The nullomers disclosed herein can distinguish other cancer features, for example, subtype of cancer. Non-limiting examples of nullomers that can distinguish BRCA and non-BRCA breast cancers are provided in TABLE 7 and TABLE 8.

TABLE 7
Non-BRCA predictive nullomers.

cctgcatgcggatgtt, cttgacacacccctca, cgcggggtggacttcg, acagttgggcggccgg,
agggttttttactact, ttacagtctgcatata, gacattaaacccaagg, tgacaagctagattcg,
ctactccccccccggg, tccagtagtttgttta, tcactttacccacaag, ttaataagacaggaat,
ttgaacttataggaac, ggcggaccttcctctc, acgtattaatgaggca, tgcctctaatgaccaa,
attcgggggggggtcc, ttgctatgattgggtt, tgagtctcaccctccg, ttatgactcttgcagc,
tgatacggaatttcaa, ccttagggcagggcag, tgttcagtctagacta, actacatacacctgcc,
agaaacacgatcagat, gagtgggggggcacat, agggtgatcatagaga, cgcacctgccgtgccg,
tgtccagtgattcggg, ggccatgtttatgata, gttatacatgtagttg, tcatgggtttaaccta,
accttatacatgagat, gttttgccaccgtatt, gacctaaacgcttcac, ggaccattaagatatt,
ttttatatgaaccgtt, gatccccatgtccctt, gaggggtagtttgaga, cgtagtgactaagcat,
catttgaggggctcaa, aactaaactcccatag, ttaagactttgatagt, acaatgggttttggta,
aaagacccatcaatga, aaggtgtaatatcccg, accgtacccaccgcct, gacactaggggagaag,
tgtctttatatatcga, agacagatttccggtg, acacgtctactctagt, gagccgactttctcac,
cagacaaggcgtgagg, ggacagtacggtgatt, ttagtgaacgcaaaga, tagccataaaggtcct,
acgcttctgttctctc, ttgtggctgtgtctac, cgttctcatcttcttt, aatattttacatatcg,
ccatgatagaacatag, ctaggtgaagagatcg, cattagggtgttagca, ttctgccaagtcagta,
tctgatccagatggcc, cactggcttacaatca, gtgttcgggtcccggt, gtcaatatgtgcttaa,
ttacttagggaagagg, tcctccccccccaaca, ctcgtgttatttgcaa, gagaccacacttccac,
cggtggcgtgatctga, tcctatgggacagggt, agtgggggggcaaata, gactaacccaatatac,
ccattctactggtcca, ttcggggatggagttt, tgcttttgtcgttgtg, gggagaacagacccgg,
gccccttctgtaccct, tcttgttttgcggtgt, tactaaactctgtctg, acccatgtaacattaa,
ctacactegagagact, cttctctactggccca, gtcacgctttttttta, cgctccgacgccggct,
ttgatgcttgggttcc, agatgattcttcatac, gctgttagcccagatt, cgctcgagtacgagcg,
ggtatatgctgcttag, tatgaagtagagccat, acctcggaaacgcggg, ggactgctgattaccc,
aaagatggacgcaagt, cgtggctttgtggaaa, ggtatgaacagattga, atattattgagtctcg,
ggtctaaaaaaaatgc, atgaaaccccttacaa, tcatgtactcacataa, agagaggagctatgag,
cggtcacacatgatag, tgacagcgtgtaacag, aacgcactgagcccct, ttcgggggggggtccc,
tagccccccctgtgac, tgcaagtttggcatgg, ttagtcccaggtacaa, gaaggatataaggtga,
ggaggtacgaaaaaac, atctctctgttgtgtg, tgaagatgccctacat, gttaccctgggggaaa,
tttccacaggttacct, cgaaatatcccttttc, tgagcctcacggggta, gcagaagttagtagat,
tatagttaaggagatc, agcttggttaggtgtg, gactgcaaccccctca, gatggggcattaatga,
tttcgtattaacaaaa, ggggttttatggtgca, tagttcttattagatc, cactccgttccaactg,
ccagttcatagattat, ggggggcaaaaaaaac, ctagtggggctgttag, gggggggggaggcttt,
cctaggtgttttaact, atgggtgctcaaggcc, ttaaaaacgtgtacat, gctgcctaaaaaaaac,
catggaaaattaatcg, agtattatcaagacac, acttcgcataaagctt, ctttagtagtgaatct,
ggcagcgggaggccaa, aaagctagggggggag, tccccccccgactttc, ctcctcgagtttaaaa,
acaggtagagccagaa, agctggccgggcggtg, atgccatcttgtgcac, ttcctgaagaaccatt,
gcttcgggcaccagtg, agtgtgtatatctctg, agattgtgacgttaag, gcttttaccctcgctt,
ccaacgaggctactga, tgatcaagtccggcag, ccggggggggggcagc, ttttccctcttatcgc,
aggtccttagtatgac, tggtagtctatactgg, tcgtacagtgggctgt, atttgaatggtggagg,
gctaattaagggtgac, acagcatcacgaacag, ttggcgacagagttag, ggatgaactggtgtaa,
actctttgggctcggt, tagctgtctaaattgt, ccaggttaagccattt, tctacccaagggtacc,
cactggcgagtccaac, ctagtttgtatgaccc, ctagcttccacctatg, gtctttaactcaaata,
taaatgcgtttccctc, ctcaggtcagggatcg, gagttctaaaaaaaac, tagtgtgtccttgtat,
cgtgatccgctgccct, ctatgtatccctctat, tagcgtaaaaaaatac, cactttagtgtttgat,
gtccatgcatgcttgc, taggaggcttaggtag, atagctcccccccccg, ctctaaccagcagtgc,
ttcaaaaaagatatcg, atgtttgctcataccc, cttagagagtgaagaa, ctctgcttagagctgt,
tcccaacgtcacatga, ggaagccgcggtacag, aggtgactaccaggtg, gcaggatgtatactga,
atcaagccaggtacac, caggaatggagtctct, tgtggtatgtttgtga, gctgccgacccctcca,
cgccgggccttaatta, tccacacatcttggtc, ccccctaagggggtgc, aggattaaggactgaa,
catggggggtatacta, accccccaggtaattt, tgttataagtgaaacg, tgtagatgttatgctg,
gcctaaaaaactgtat, tgaggagggggggggc, acacgaagaagaagat, ttcaggtatgtggatc,
catgtgtcaggactca, ggatgtcatccaagtg, tgcagtttcatgaaca, tcgctgctgtgtcctg,
gggttccgtcttaaaa, caacatgcctttattt, tcttgtcgtacaggct, gtcagtactaggataa,
ctctacaaagagccca, acacactgctaagtta, acgtaaggagtcatgg, agggggtgtaatttaa,
ataaaccagatcgctt, agtgttgggtagttct, gtccgagggcggcctt, cacccttaccagcctc,
agcttcaccttcagtc, gcatattactaggctg, gcggaatggggtaagt, cctgtttatcaatgtg,
tcgcgcacggtgcgta, cagactccatctgaag, agagagtaattaatgt, atagtttgcacatgtt,
tacccacttacttaaa, acgcaaagcaagcaag, gggcccagcacttttg, accagaggctgactaa,
tcgggtgtctccctgt, gacttttccctccacc, ctacaaaaagacagcg, cgtcttgcgggtgaga,
acccacttgtagatag, acatccaggtgacatc, tgccggcgcttttcag, aggcgggcttagtcca,
tcatgggggggaagca, catgttcctcaagact, cttagtgcaacgctgc, ccaaaaaaacaggcta,
gagaagtcttatgtag, tccgctctgcctggta, aatgaaaaaaatcgtc, gcaatgtaagactgaa,
gcgcattccgcgctcc, tgtgagtgtctacacc, gtgacaaggagatgac, ccgaggtatcaagcca,
tttttttctagcacga, cacagcaaggcccgta, gtttgtgtaatgattg, gtcaggagagcgagcc,
tagtccggttcacttg, ggccgtaaaaaaaaca, tatggttaaaagatac, tgcttcttccgccact,
ctcggggctagagtgg, ccctctgaagcctegt, taaaaagacccccccc, gagtggataacgaaat,
agtacgaaatctctta, gaagttctggggtagt, atacgagtctccctct, ggtgaccccactcctt,
ccctgtgttatgaata, tttgacgagcttagag, catattgtggagctaa, ttacccaactcggcct,
agacagtatagggaga, aagccgggcagctcgt, gacgcatatactttgt, attacacttcagtttg,
accatgaacttgcttg, gagatattaaaaaccc, catacgacagcatgta, tatcattttgtctgct,
agcgagagcttatatc, tcactaaatatcaagg, taaaggaacctttaga, ggggaaaaaaacgtgg,
gaaggtctaaaaaaac, cctaggatgttcttat, ctggcatacgattgtt, ctctgtgcgcctgtct,
tgatcttgcgaccaac, ctgggggacagttaga, ctaaggctgcggcttc, ccttaggcattgtagg,
tcgggtcccggtgtgc, gataaaatgcctcggc, actggagaagctatgg, ggactgtgggattaac,
cagtgctctcttagta, ccttatcgaggcttca, tctaaccatcccagtg, acataactgtacaatg,
atgaacatttagacct, cttacctatgtatagc, ggcacgaaaaaaagag, actgtaacataagggg,
aattcgatatatgtag, ctcagtgttattacat, gatacatagctgtaag, tttctcctattgagtc,
gcgactctcccgccac, gatacgcgagcccaag, atatgtactacctgaa, catcgttcctccctcc,
ggacttttatgatatt, catgtccaccaaacga, cttgtgtccctcggaa, tcacttcaagagtgca,
tagcttagagcggttc, ttggacacgaaagaaa, ggaaatcataagtcgg, attgttcagtccctac,
gctgtggcatggtata, cctcataatcgcagtc, tagtcactgtatgttg, ctttatagcatcatgt,
atgtccactactaaac, gctgaatagccaaggg, gactcagagaatctat, ctctttcacgagcaaa,
agaccatgcaggatcg, gcttctaataatacac, ctccaatcccgttcaa, gggtcacggccggggt,
ggttaacaataatcac, gacacgtggtgctaat, accccgaaaaaaaagt, aaggcctttttttggc,
acccggctctaacatg, accagtgctaagtgac, atgaaaaaaaggggtc, aaatcgcattccagca,
ttccccagcggccgcc, agtttgcagcattata, cctttgtcagaggagg, agagacgacaagagat,
gaggatgtatgtctct, gctggatgatatggag, tctctgtatagaagcg, gcggttttattattaa,
taaccgcttacccagc, cgttttttttagctct, ccccacttctgagggg, tgaaaaggttactagt,
gcactgagatcacatg, agtgttcagtagggat, ggacccacggggtttc, ctgtcctaactgaggt,
ccggcctacacttagg, gctgtgctcgtggtag, aaaaacggggattgtc, gaggctattgagggca,
ctgcagattagtgact, gaaggttgcttaatta, aggaagccttgcccgg, agcttttttttaggct,
gactatatgcgtacca, acttctaggctttcat, gaagtagcagttgtgc, tctcacattggcctcg,
ccaaacatcttgtata, cacttttctaggccga, acttaagttcagagat, aatgttctcgttctaa,
caccaaacagataagg, gattcgtcacaattat, aaaaaacggggattgt, cagaggagttaggtag,
cctcctgttctctcgt, gttttaacagctcggt, ctgttgtaatcactta, gcccccgaatgaaccc,
gttatttgtgtggggc, cttaggctggacgtct, gtttttttacagtatt, tgattcgattttgaca,
aatgagtttgacgacc, tcatggacggggcggc, tgggtgcttgcaccat, accaggttcctatact,
tatgttatggcttaga, ggaacgtccgtcagcg, ggggcagaggataaat, cttggccaaactgttg,
cagggcatcacctaac, tgcttgcgcaggggcc, ttgcggggggcgcctc, gaccctttttttaggt,
gttcacagcctttact, aggagacttgctatca, tagcgggactacatgc, gtctgccaagatcccg,
tccaccccctegaccc, ccgctccccccactta, cacatgacaacctgcc, cagtttggcataatca,
ctccgcacctctgcct, ggttttttttaccctc, tcaacgacttgtgaca, gggccgcgttttcacc,
gcctgattgcctctgt, aaccactgcattgccg, tgcagaaggggatgac, gggatcaaaaaaaacc,
ttatgggtagttaggg, gccgctgctttatgaa, actagaacacttggac, ttaatattcccaccta,
tgaatcaagatatgcc, acagctcaagcaagga, caaagatcatttccgg, aattcttacgggctta,
tgccttgccacatctg, ctcgtgtaacccacat, gtcctaacatctataa, gagaatgtgctgaaat,
ggagcttggagatctt, cgcgaccccgccctgc, cagatttgtaaagtgc, ggtgacacccacagcc,
tataccattagcagtg, ttcagttcgcccaaga, ccacctcatcgtcagt, cttcactcactctggg,
ggcatctaggggggtg, ccctgtatacttagca, gggcacttgctagtgc, cggaacacaggggtgc,
gaacttttgcactcgg, tctgtttactccgata, ggggggggggtaccct, aacgacttgtgacaac,
tccagttgttagttgc, tattgggaaaggcccg, gggcatcccactgcct, gggaacggatcctggg,
caacttgagtcagcct, acggaagctcagaagt, tgccaaccctgtaacc, acgcgggggggccttt,
gttaaactacgtctca, ccacataaaaaaaagc, gagagatacttatact, tagattctaatttgct,
tttgagtgtacccttc, tactgggctcaaacac, ggcgtgagatttaata, gaatgtctgctggccc,
ctgaagatttgccgta, gaactgcaggtgatac, ttaatggtagagttta, acacatactcaatgat,
tctcctatgtactgtc, gcattcggccagtcct, cctgcttgctcaccgg, atttgagttaaccatg,
cggccccaagttgtgt, ttaattaaaaatcgga, ttcgcaatatattttc, cttaggctgttgatgt,
gttgccgtagtccggg, tgaagcattcgttaca, cgccttggtcaaaccc, tgcgctttatttttta,
ctttccaggtccttat, attgtcttatgcccac, agagctactaacctgt, tctgttaaagggtgta,
tcagtggctacccatc, gtggcattttagatca, cgtgggggtctccctg, gccacctggcataaag,
gagttggacagtccta, tgtgcacttcgtcagg, gcttatgataaacttg, gacggcactcacacct,
tatattacagcatcac, gaatttaaggtccctc, tgtaatccctgacctt, tatatccggcatgtag,
aacgttttgtccggag, cctcgcttctgccttt, aagtgggggggggctc, cagtacggtgatttta,
cttgactatcttttat, ggcttctagcagcttg, tacggctgggctcggt, acttttttaccactct,
ctgctcggaaggctta, ttcatcctcttgctta, tgctaataatggtctg, taataggtaggtttta,
tacttcttcaaattgc, tggttacagattggct, tattcatcgacacatg, ccccatatgctgtata,
tgcactttagcaccga, tgattcatctcctggt, cgcctttgatagactt, ggctgcggcagtggtt,
gatggcttagaacacg, actaccctggtacatc, accctagcatttactt, tcaactacaatcacac,
cgacagatagagaaca, ctcttagctaattgtt, ggattaaatctcaggc, taatgggcgtgagcta,
ctagaaatccctctac, agccagcctgcacgag, agaccctggttccaat, cgtaaaatagatatgt,
cagaggtaagtgcagc, gggtgttagctaagtt, tatccctggtgagagc, gccacagtcatgggct,
gggttgaaagctatcc, ctctgtcagatggccg, tagtgcaacgctgcac, cgttaaaaaatggaat,
tatcaatttggcaata, acgtagactacaattc, gaaacggagtctctta, attatgcagtagtaat,
catagatagatctgta, gcacttttagaggctt, ttgagctacctcgccc, atgcccggctacctgt,
gccatataccaaatct, ctgtaagttaagttgt, ggccttgtgactgatg, ctgtgggttcatttgg,
aaatcataagtcggct, taaaaaaaacgcacac, cttagcctccaacacg, cccgcttagcgcagct,
tagaacataaaagccg, ccctgtaccaggctgg, ttagggtctccaaaac, cgtggtgcgtggccct,
ctcactgtcggctatt, ttcgatattctgatag, tctcacagtgtaaggt, ggcgctctggaacact,
agactccatcaactta, gcatctgacacaccgt, tactatttgtgggact, cctcagtcgtccaagt,
cattcccccctattct, attttgtcttaggcgc, tataacgtttagtgat, actagccttcttgaga,
gacctaaccacagcac, gtgctctgtaaccgct, acatatttgggcctga, ggggggggtaccctca,
ctcaatgtgaattcgg, agatgatgccggagca, atcagttccaccccgg, tcaacttatgttcact,
agagtaggcgggggct, tggccttacagggttg, cttgtaatcttcctgc, tcccgtcaacagtgcc,
ccccaggtaaaggata, tcaaggaagcatagag, catctccactgtgact, gatctcgtggcccgag,
tcaaaaaaaacgcaac, tgaatcaacgacttgt, ctcacagttggtctct, atgatatatagcttca,
tggtggtcatagaaag, gcattggaactgttat, tcctacttgacgaata, gcttagcccttcctct,
gaggtcacggtgaacg, gctgggtgttagccac, agcggttaacatgcct, gcagacgaaaaaaatc,
ctcgcttttattttac, cccttaatcacagcac, aattgaatcagcgtaa, tcctaggttaaataac,
cgattcttccccccct, cacaaacattgcatac, agggacagagctcttt, gcttcttaggcgatcg,
aatgacctcagtgggc, tgatatggtcactcag, gtttacaagttcacag, cacccccccccacgcc,
gcttttttcaacactc, tgaccaggatctccac, gacacactcgtgtata, gcttagagctatcagc,
aaacctgacctcggag, tgccctccaccgcgcc, actcgcagttagggat, atgattccaatgggtg,
tcggacgtttggggct, ccttgccgcgcccgcg, catacgctttttttaa, tgcgcaagagacatga,
tttccctttcctcgcc, ctgaatattgcgcagg, acagtatagtaaagtg, gggagaggatcgctga,
ggtccatgactagcag, tttgccggcttagagg, gggacaagagtcacgc, ctccttcgtctaactc,
agaccatagaaaccct, ctgacatccccgtcct, agttaaacatacccac, tcaaaaaaaaccgtta,
caagccatttagcccg, gatgcgcagtgtaact, gggaaattggtggaaa, tgcgtagcccggctat,
aattattaagttcaac, caccccgggtgggggg, gaggtactttttttag, ctgtcccgatgcatgc,
gtaaatagataccact, ctccttacctcgagat, ttaacactgtcaataa, aagttgggtgaatccc,
caccacacttagagct, atggtatgttgggtgc, caagaaatagtgggtt, aacccccccatgctat,
caatctcccctaaggg, ggtgattcaactgtat, ccatacaaaggatatg, tgttttttagggggcc,
acttgccctgagcacc, tagtcccaacgactct, acctgaacattcacgt, gcatgagttgatagcg,
acccacgaaaaaaacc, agtcacgggaggttgc, caatacaccaatatga, atataaggctccagta,
ctgaggggtcagctag, gtccagttggaagaat, ccccttggggctgcgg, tgtcgcatgcctgtga,
tggtcaagctgggcga, aatctgagtcccagtc, aagggagcttaaattc, cctttggcaaaactgc,
attggtgcacaatatc, aatgtgtttttcccgc, tttaaggcagaatgct, ttttgactcgaaagga,
aaacccctgcaatcac, tcaggaagcttaggcg, atacaaccactaggaa, tactgttcgggttatg,
taatgttaaccttaga, agtattacagagaacc, gcatagtaaatcacaa, ttgtaggaactatatc,
ttggactcagcccgac, agtcttttttacaggt, agcgaaacatgctctg, gatatcagcattcatt,
accagatgttgaacag, ggtgggggggaccagt, tgatgggaactataag, ttacttggccccctgc,
ccaaggggggggtcac, gctggggggtcaactt, tttatttctacggtgg, ctttttatgcatgcag,
tcagacatgcctctgc, actggcgagtccaacg, tcattttttgtcgatg, cagttctaatcctgcg,
tatatacaacatcatt, aattgaatactgctaa, gttcctttactcccag, tccttgactgcaacat,
ttgcttaatgacgtct, tgatgggctcatgtag, caaagagattcgtaca, atcctgtttcgagggc,
ccgtttttttggtaag, gggggtcagatgccta, tcagtgggctgactta, aactctgtggtcacga,
ctgcttgattaacatt, aagctctggccgtgga, aaagaaacccttgaat, agtgtgcctgatgaca,
ctgattcctttaaatt, cctctcgtgcggagcc, ctagccggggatctct, tgccccgcggcttgtg,
atatctttggtggata, gaagagggtttgaccc, taatgacaagggccgt, acccctaatgtctgga,
gtcaggagaagtataa, gttttccctcttatcg, ttgttcacgtcggctt, gactggtgtggggggg,
cccccccccaacatgg, agtgtaactgctatat, caggtaggtgaagacc, ctcaggactcgagcca,
ttatactgagaaggtc, ggtggaacagtagctg, aaaagggcacgaccta, ctccccccctaaagtt,
ageggctctccacctc, tacccggcaaaactat, gatcaagatacaaaca, ggattataggctccta,
tcctccgacagattat, actaaagcagataccc, attgacacttgatcta, aaaagggacttatgag,
aaacctaataatgtcg, ttggtagcctgtgggc, tactccccccccgggt, tgaggggggggactaa,
ccatttccggtaaact, atttaaatgccccggt, ggggggggacttttga, tattagggggtgaagt,
ttaccgattgtcttta, tgagtgcgaacgtgca, acgtgttatgtaataa, gctgtgtttgtaccaa,
tcacacataagctcgg, ttgtcgccatgactcc, gtcagggccactgaac, gtgaaatcttagacag,
tcatgtcagcgattgg, ggaactgccatgtcac, tccagacgctccgcct, aaaaacttgatacaac,
cgggccggccttgctt, acgcccccccccacca, ggtatacgatacaata, ttggggggggttcacc,
agactcacacttagta, ttccctcttatcgccc, ttttcatgcaggccta, ctgaacatccttagat,
aaggtaagaagtgagt, agtatggggcctgggc, gatgacaagataaatc, cttagcgccgcgcagc,
tgtctaagggggggga, atctaatatgggtcat, tacaatctgacgtttt, gccaaaaatggttgct,
gacagttgggcggccg, acttaggttggattat, agaagttggtgatgct, tgatgcacagactatg,
ggcttggcttattacc, ttcagcccacctttag, gagaagtttgataatc, gtgaggcttatacatc,
tttcataaggttgagc, tataacaggtatgctt, ccagttaggtttttga, acgacacttcccttgg,
aggtggggggggcctt, gtatataagtcttaac, atccccatcatccgat, aacgttaggactctca,
gggggccccccgagtg, cacaaggtcagattag, tgctttctccgtgtag, cccaccagtaaacgcg,
cactaggctgccgcta, tcccccccaatttaca, cggcctttctcttgtt, aatgaggacggggcct,
cacctgtttggagata, tcccccccccctctac, ttgaccctgcatgcag, atttgcaaccataccc,
atttcatctcccaaga, cctaggtgaagcagcc, tattcgggagacttag, ctaatgagctttttag,
ccgtgagttagagcag, gttattaaactcagat, gtgcatcatatgcagt, tgagctctcgtcttcc,
aatgtacaaacaatac, aaagtcccaccaggta, ctcataaatatctacg, gggacaaaaaacgaca,
gtggtcttatgtaatt, gccagatgtagaaggc, acaggcaatgtctgcc, ccaggactggcaagta,
caagatttcggagttg, gcttggtggcgctcgc, ctaaatgtggtttgtc, ggttttcgggtttggt,
cactccatcttggcga, tcaggggcacagtccc, cttgcgaccgccctgg, ctatatcaaaaacatc,
aagaggacttctctac, tctgtctcgaaacaca, cactagtgccagggtt, gtcagcccttttagga,
tgtaatagtagtattt, ttatcaaagagaggga, gcttaggataactggg, tcgtatactgagtcca,
atcatgccccccccca, gggtggaactgggacc, actaggtgtgactata, agcttagtgactctag,
ggtatatccaatgaga, actcccaacgtcacat, tctgattgtagacctg, gagccaagagcttagc,
ggtgtacggtacacca, gcctagagcctctaat, ggccaatagggagcaa, tgtaaaggagccatgg,
ccctttatctgagagg, gcagcgattgtaggaa, ttaggatcaggcttat, ctctatcaatgagttc,
tccagtggggggcaaa, ttagcgtttcctacag, ctactcgggcagctta, atgtaaaaaaaatatc,
aaaagacctccccccg, aggatcatggctgctc, gtcagatgcctacagt, cttccggagggtaaag,
ccttaatacccttgaa, tcgaaattagggataa, aacatgagcccccgaa, agtagatgatcataag,
tacattcatgtcctgg, cgctttgtcttctttc, tgcgcagaaattcaga, agaagacaggatcagc,
gggtgacagagcgtta, aaccgattagaatgac, tctgttatgctcttgg, ggcttaaagccagcac,
tcgggggggggtccct, ttgacaacactaaacc, atctgagatcctccta, ggcgctaggcggctgc,
cttatttgttaggtgg, tttgtggtagaacaag, tgcctcaactgatttc, tcgtagttagatgaaa,
attgagttagggtgca, tgcagcataccagtaa, gtgagcatgctatgca, ataatagctaggctat,
atgaccttttttaatc, ttcctttgggccaata, ctccgtagagggtttc, accctaccgcacagag,
gcaaggtttgtgcctt, gtggacgctaccggcc, ttatctttccaaaccg, attaggctaccattga,
gaatctcagactaggt, gatgggtgcaccccaa, gccctctagccacccc, ggcacttcccgtctga,
ctttactcgcctcaac, aatagactaactgtct, gggccggataaggaag, tgagtacggaactcat,
tgttataaatgcgttg, gtatgtggtttcaggc, gcctgctgtgcaagtt, cctgttaggttatggc,
ggtggagataaccttt, tgcgcagctgtcggtt, attgtctttgcactca, gtgccaggccatagtg,
atcaatatgtggggat, cttagtcttttggcca, caaagatgtactaata, cgattttaaaaccagt,
ggctatttttttagca, tgatgtaagcctgtct, ttgttgctaaattgag, agttcatctggaaccg,
ttggcccggagaggac, tggcggtgaagttcat, aatggggggtatcagt, agaagattaggtggga,
tagtttttccccaagc, atttcaactcttcttg, agaaggcccatgtttc, atcccttgagtaaaag,
acgtatcagttggtga, ggaagctgacatcaaa, tagtaattgtcagtgg, tataacacccttatta,
ggataaaaactctgtg, gcccgaggcagatcta, caccgatgcttaggag, gcccaatgtgacaggg,
ggcagtagctggtaat, taactgcttaacttat, ggaaggttttactggt, ctttaagtgttcgtta,
tgtgccaaaaattgct, ttccctacagacgtgc, tgtacctccatctctg, ggctgtcctgtaacct,
tactcattaatgtcgc, ggggcaaaaaaggcac, actagcacaatgtatg, tctccctttgatgccg,
taacttgaattcctaa, tgctggtggtcgcatg, agggaaccaatgaaat, taatgcctcaactata,
tatctttgctctatca, cagctttactccttcg, ttgcaggaccggatga, gatgcttttatctaac,
aaaagcagaaaaacgg, tttgtagattacctgt, tggaactgccatgtca, ttacaaaaaaacggtc,
cttaggtaccgggctc, gctcgtctcctcatcc, tagcagcctttaatcc, gcataaaggggtggat,
ccttttttgcggacgt, ccaggtgctgctatct, caactagggatgttag, gagttattaggggcat,
gtctccgcctccgcag, cccctaagggggtgca, ggcagtcagcttgtcc, gcaacctccttcgctg,
agcaacctttcaacat, taaccgtatattataa, gatttccaccaccacg, tttttaacgaatctta,
ttggcctgtctgagag, cctgacctataaagtc, gcataattgaggtgtc, tggctagagagattaa,
cacctaaaaagatttg, agacaagctacagcgt, ggctagctaaccttag, cctgggcgacaggtcg,
aacctggcgtagtggt, cctgattatcatggat, acgtaagaaaaaaaac, aaaaccccacacttat,
gagaagtatacctgct, aaatggggggtatcag, aagtgacgatacgcga, ggtctgaccctgttgc,
ttacctactacgtcct, acaggcgcgtttcacc, tgcaggcttagtgatg, tactgagacaataaga,
attagtccggttcact, cctggtggtgaagcta, cttatagctgagacat, gtcccagattgataca,
atgatggggggggagg, ctacgccggcggctga, ttgttttggtgcagcc, tcctcgagattcctgt,
tagttcatagtaaggg, tgtaaccgcttaccca, ggcatgctgaggggtt, aaccattactaatttc,
actataatgggagtcc, ctttcttccgtacaat, tgtcagtgagtacagc, tcaggctgcactctaa,
atattaggatctgcag, gcaagagagaattcgt, tttaagccctcctggg, ataatagtgttctcta,
attcctggggtttatt, atgttaacatgacagc, cttggtggcgctcgcc, gcagaccgggaaagat,
tgcctcactataaaaa, tggtatgtctacctac, cctagggaacatggtt, ctagccacagctacag,
ctatgtcccttagact, gaatacttcttgttaa, atcaaaaaaacgtaaa, gatgtagggatatgcc,
aataacagcgtgaacc, ggggggggaagagtca, tactccattaacctta, cttagtctcatggaac,
ccccgaaggcaagatg, aacgcacaacttataa, acattactatggattg, tttgcctgattgggct,
aaaccactattcatgg, cagacaccggggatga, gctcctcaaccttagg, cctgtgctatatacgc,
gagcaaccatgtcctt, tccccccccattocca, agacaccggcctaggc, taacttatggtcagga,
caagtagaagatagcc, ttgaacattatttgag, tctcaatgtgtatgtc, gtctacataacctgga,
ggaaaagctcctatga, agctttctgcgtttca, aagaggaatctcttag, ccttcacgcaggctgg,
cgcaaaaaaatacaac, aagactaatagtgaaa, acgcccacggattctc, agtctttaattcaggg,
ctggccctatggcccg, cttggacccttttagg, tcgattttaaaaccag, ggttatcactgtgtca,
taagcctaccccccat, atcttaatggaaagcc, aagtaatcataggttc, tggtacgtggtatatc,
ccccgaaaaaaaagtt, agattagacatacagt, aagtgtgtccggaaca, gataatagactgttga,
tcatattccttgccag, cggggggggggtatca, tgtagttttgcacccc, tcgaaaagtgtttgtc,
aggtctcaccagcaat, ggggataagacctttc, atggttgctggcacct, gccaggctattctcta,
cctttttatttctcgt, cttggcttgaagtgtg, gctacgccttctgggt, atggagattcttactt,
tacaaaaaaacggtct, gatccactggctagaa, gcacccccccattttg, atcatctattagaggc,
ctattctccgcaaact, tcatcaactatattgg, ccaattcagtcttccc, cctccacatagggacc,
tattagctcttgctta, taaaccatgcatggac, aagcccgatgcaagtc, gaaattactctatcta,
attccgatccttggcc, cactctctgtcagtga, ctactcgggatgctgc, tgatatgtcacataac,
ggctagaccccgtttc, atggtctgacagtagt, acagattaagctagaa, cccaaaatctgctggc,
agtaagagccctcggt, cttcttatattatatg, tgcgatttgcctgttt, ttaccctaggtttgtg,
tatgtttcacatccac, gcaaaacagttagact, ctgcctaaaaaaacct, tgatttgttatccttc,
gtgccttttgtgggct, attcgtgacatattta, taagtcccccaagtcc, agtgttctgaaaggta,
cgaaaatggacttgct, gcaattctccccaatt, tatgggatcagaagct, tagatatgttcaccct,
ggacccaggcgtggcg, cacccatcacagggct, gcccattgggagcggc, gttcagattagttatc,
tgttgtttgtccctga, cttgcactttgggcac, cgcatctgatgaaacg, gactgtaaatttatga,
gtccatggacacttcg, ttcggatatctgttac, cggatgtatacagtaa, agatggcgccgggatc,
gatctgccctcgcccc, tagtgcctcagagact, tgagcctcgtgagtag, ttttggctttgcgtta,
atataggccctatttt, ttcttccgtacaatgt, aagttgtcctctgcct, gacagtacggtgattt,
gaaatgctataagtaa, agctgaaatcaagagc, agcctcatgattcgac, ttgcaaattgcgcagt,
acctcgagggtcctcg, ctgacccagggtctta, tattaggccgggctcg, cagtcttaccctgtcg,
ccgcgtcaggctgttc, aagccccctgcagtgc, tgtcccgatgcatgcc, ccggataatagtttaa,
tggtccaccatgtggt, ggtctagttcatcctc, tgaatcacctcaagga, taattgataaccagat,
cttcttgaattacaca, tacacagctctcacca, ctaatcttcaaggtgt, ggctgactatgtttct,
gcgtccgcctccatgc, aaagtagtctactatt, aaccctcttccgtgac, gggcaggtttttttta,
agactcggggggggga, tcgtcttaagtatgtt, accctccttttcatgt, aattctggcacgttta,
acgtctcatgtcaact, agcaagtacgatgtct, gtgtgcaactttcata, aaaaaagctctcttat,
ccgtgccgcccgccag, tctgagtgacatatga, actcccctgcagtatg, caaagctcggaagata,
aatgattcttccaccg, ttagtaaggaacctaa, tctccccccctaaagt, tcgcgccagcagccgg,
aacgtgaggagcgtct, gcaatgttttttttat, gtgactatgaacttcg, gaccagtttggcctcg,
ggcgtggcggccatga, tggtggcggtcacctc, aatgtgtagaattcac, gctaaagatgtccaag,
cgtggaagaagcatgt, tggtccttgatggtga, gaaatcattttccgaa, ggcacatcaaggtgta,
gcgcccatttccctgt, tgaaagtgctgcacac, ggctacttatgtgaaa, gagatgcagtagcacg,
actgtagtagaacttg, gtcccccccatcattc, cctcactgcgacgtct, ggcttcccttaatatc,
gatgttattgggaagc, gacctaggcctcagga, tctagacctaatgtgt, aggttgctgtcgacct,
ccctcaagcgctgcgc, agctgtggaaactttg, aattcttaacatcaag, gaataacttatgttga,
cttctcattcgtcatc, cttaagtgttggacag, gtaaaattaaacggat, caccagtaaacgcggc,
cagctcgtggacaaaa, tctcgcacagcaatac, cggagtctgacgctgt, cgtacacctgtaattg,
ccagttccctgcacac, atattttacgggaggg, ttcccctccgtatgtc, aatcttaggtaggtga,
agtgggcatgcggaat, tgatcagcgtcaatgc, atcgaaatatttaccc, ggcttggtagttatga,
ttgataactcgctgtt, tggtgtaaatcgtgtg, acctttggtagttccc, acatgcaccaaaagtg,
tttaacccatgagtaa, ttacaggtgattcacc, attacatgtcagtctt, ctgtggcaaggagtac,
gcaacagatatatggt, taaatgttagtctggg, ctggtagtcagtctta, ttgctccagtcagtcc,
tattttgctgatccgc, ggggagacttaagttt, cggggggggttcccca, tcctaaaaaaagacta,
gctaaatgcatctgct, tegtaaacctaagccc, gcaaactgaaggtaat, gccccccgaggtacct,
aggactctagagagtc, acggattgatatggct, tgcggagacagtatgt, agcgttcaccccagga,
tgctttttttcggcca, tgagggctaggggggc, aatgttagtccattaa, agtgagattagctaac,
gagaatgatatgcgta, tgcttgtgacactttc, tgccgttttttttaca, tttatcttacgggctg,
tggaacgtccgtcagc, gcttaggtacgagaat, ggtctcctcgagcaca, tgtaggaactatatca,
aagtagcgcgaggccc, attggctaactacact, acagcctttttttagg, ttgtttggccttctga,
agtgcaacgctgcaca, ctccacggctcagtgc, tgctccgccttactat, ggtgttacatatatct,
cccgtgtctgccccaa, acaagagtcacgcacc, tagcccatatctactg, ctagggaatatacaga,
taaaatcgttcatagt, ccaggttgagatagtc, agtacaaaaaaaaggg, gcagatggcccacttg,
attaggccttaagacc, taggcctgagccctgc, cgtgcacagcatgctg, actaaaaaaagtgcct,
ataaaaagaccccccc, tcatactggccatcag, atatcaaggctagaag, aaggatgtgagttgac,
attccataatactcag, ccccgaaaaaattgcc, cccaacattaagtctc, tttgagtgtaagtctc,
cttggaacgaggtaga, actaggaagtgctcta, gatccacaaaaaagtc, aattcagcggtgcggt,
cgaaggtcaaagagtt, tacaccctcttaagga, tcaggtcaaggacgca, tgcgcttgtccacact,
tgtgcgcatcgtgcgc, ccccccgaaaaaattt, acgtcacgcttttttt, gtccctcagatccatc,
ggactttttttgtgcc, acggggcatttttttg, ccttgtccacctacca, gctggactttaactct,
gcttcagtttgtaagg, taacaaacgttatcct, cagttactcgtaaggc, tgcgtgtctgttttct,
ggcaccttgtgccccg, ccccgggcgcactatt, gatcctcgctggattc, agaacactgccgagtt,
ggagtggttagacagg, gtctttcctcattgcc, tccaatccctgatgtg, gccttgcccatcggga,
catgcgcgctcgcgcg, tcggttttagaagtac, ctgcccgctcatctgt, ccttgcgttcgcccaa,
gagttaaccaaagagg, tgccattacttaatgc, gttgcacccgtgcagg, ttgatcacaatatgat,
gcgtaggataaaatgc, tttctcactctcttcg, tactcccacctattaa, tgcaaaaaagcgtact,
cgttgaatttacctgg, cagacgctccgcctta, cttagggagttgatac, ccatggtgttcttgtt,
cactggttatgtctcc, gaatgaccattacaat, atcagctcgtggacaa, gccccccccaagggct,
agcagacattaggtcc, tctagagatatatgca, aaagcttgccccttac, gcaaatggcggtgaag,
ttgataataagaaacg, ctaaatatgatagcga, tatttaatcggttgtc, ggctctccccttcatg,
gcaagacgcagtctta, cttcaatgttaggatt, aacaaagcatagacct, cataattccgagtcag,
gttggtacagccccta, acttgacccttagggc, gtcgcaaccttcccct, cttcaaagactcccat,
tctgcgtagcccggct, ttgtggccgggtactt, gtgcatcatcaatagg, gggaaaaaatttagac,
tggggaggtaggtttt, tatcattcgattacta, aacagagagggcaatt, gaacagacgtgatccc,
taggctgccgctagcg, cccttactgtaaattc, atgagatgtgatgcct, attcttcttcgtaatt,
agtggaggggggggtg, ccctaggggcaccacg, ttctttaaatagcccg, accttcacgatctctt,
agttaccgtaagctcg, cggactgcagtgtcgc, tgctcccgtcaacagt, actgtttgttcaattg,
gaatggacaagctggt, gactccgtagagggtt, tggggggggaggcaaa, tgtagatagaacttaa,
cttaacccacactgtg, tttttcgtcacccccc, cattacatgtgtgctt, tagcccaaaaaattgt,
ttcgttatctgtggtt, aacatacattactgct, ttagcttaccttgtga, gtagtggcccctctct,
taacccgggaggcata, agtcccaggttcccgg, tcttcagagtccactc, ctgtttgactagggtc,
tgtagtcctacgttag, ccatgtccaccaaacg, attacttggccccctg, ctggggggacccgagg,
ccggccactgcggctt, cgagattgggtgcttt, gcaactgtgatgcaaa, ccccttgaggctcttg,
ggagcccatgtaggga, gtacatggagggtcta, cattggcaagctaatt, cgcttagcgcagctgc,
acctcatgatccgtgc, tttgctgatccgctgt, gcccgccttagctggt, tgacattagagtaaac,
cttagattatagagcc, gtattacccacgatga, gaatagcgctagcgct, gtgcccgccacaagta,
tcttaaggggggagt , cagcacgtagaaattt, gtgcttctatgaaagt, cacaggggagacttaa,
cccgaactatttttct, actaatatggggggga, gtacacactaaaaagt, ccccataatttagaga,
ccatatggggggggat, caggttattcacccgc, atagacctcagcattt, ctctagtgcgatcacc,
ccatctcagaacgtag, cattacctatataacc, gttcagggagccaaat, cgtctttgctaggctg,
taatgaaactggcata, atctggactctttgac, actgttctagctagta, actccacaccttgtgc,
tccategttcctccct, ggtgaccctgtttcaa, cacagtgtaaggtaag, acgaaaatgctctcat,
cctaacagaagcaaag, catcctgttgagggca, gtagtgtagtgagtct, gagtggtgccttactt,
gacggggtttgaccgc, actccgtctagggaca, tttcttaggttaccca, gcacaattttctcaga,
tgtcctgtgcttagca, agtatcatgttattgt, aagcttgcatcacctt, ttacggatatatacat,
atattctatcccaaac, gaggagtagctaccac, ctgtcttatggcccaa, gatgttgctgcaatcc,
agagtggggggggcac, ataagtggggtatatt, cgacaagagatcctta, atgatagttaattcca,
atagttttttttacgt, ggttttttaagccctt, agctggctcaccagtg, tggctaaaccccgtgt,
gttagaacctataacg, cctaactgagcttaat, tagggggggggttttg, ttaggttattctgttc,
tatcatggggggtata, cagggcttaggataac, gactagggcggtttta, taactcgtcaagtggc,
ggtttgttcctcgcga, ggatgctataaaggtc, aggcccatactttaat, tctccgccctcattca,
gtaaaaaaagtaagct, acacacctgagccccg, catataggccaggata, gctagcctaatgtaga,
tggtgggtttccatga, ttgacttcatgagtaa, tgtaagcagactggat, aaatcctcggacccct,
aaacagcggaaacccc, gtggggggatgactaa, acaaaactctagttga, gggagtacttagcaga,
atagccaggtatgcta, atgctctgacggggct, ggacctctacaatatt, ttgactatgctaatac,
aacacccagcaatcct, cctagctccctgtatg, aattgtagggggggac, cctttttttccgccgc,
ggagcccaaggggggt, ggtcagatagattcac, gggcaacacaaagcga, aagtcccagtgatgct,
ggacgaaaatgagggg, ggtggtctcttcgtcg, gatcccatctgtatag, catttaaagctttggc,
cccatgagtgatattc, ttcctaacctagaggt, cattgggagcggcagc, cttattgtatgattac,
ggttgagggcgtagaa, accgctttttacagaa, ctagggaaccccccct, agggtgattcaactgt,
atcactttttttagca, agccatagggccaggg, tgataaacctccacat, agagattttctccgcc,
cgggcctgttcaagag, ccgctttccatcgttc, ttgtgcgtttttttag, cttttacctgcattgg,
aatctcgtctgtttta, tctgtcatgtacacag, tcagttattcttgcac, actttttttaccaatt,
ggtgagttccattttg, gacatctgacctgtaa, gtctgaaatccccatt, caattgatcgtctcac,
gccggctgggactcca, attttattgtcgtaag, gtgggggggtaagcag, attgcattgttcctat,
atgtgcttaaatggac, gggcgctctgatctaa, agttccaccccggggc, tcccttgtgctcatta,
ggtcctcgcacccctc, accagcgcttcatgct, tcattgtttttttacg, ccagttagaagagttc,
gaacataaagtcttac, tgagaacgggggggga, ggagttataactaaaa, ctctcccttattagac,
tggttggggggggttc, actcaccttaacattt, aagtggacgctaccgg, gcttagattacagtca,
tagtgaatcactagta, agggagggttgggaat, ggtccgcgcgagagga, tctaggagagactccc,
caatcagccggatgtg, cgtctgcacaagactg, tactacaactactcgc, ctttacccccccaact,
agcgctgtattaactt, atggcgccgggatctt, aatatgagtgcatctg, aaatgtgcgcgggcgc,
cttaggactgaatcct, cgtgatgtgcctgccg, acgataattaatggga, ttgtcttcagaagagc,
gacttagaaaggatct, cgtgggctgctccgtg, gatgtttttttagatc, tggtccacccccctta,
accctgagaggtacag, ggcccgcgctaggcag, gcaaaaccttatctat, cggccggcccagagac,
acgtccagccttaaat, agttgtccccccccaa, cgttgccaattgttgg, actaatttgagtgact,
tccgaagcagctgggg, actctatcttgcacaa, ctaaaaaaagctacat, aaaaaaacgagagtat,
gtttgtgttcactggc, aaattgcaagtaatcc, catatcctataaatcc, gggcccgcaggaatgt,
gtgacaagctagattc, ggcaggttgaccccat, ttttccccgggaaatg, gtcctgaataggacac,
gggaggaccagtggta, tgagaaacacagcgtt, ggcaggtgggcggtcc, tgacgttaagtcctcc,
atcccaaggtcagaac, catcagaggataatct, acacaactccagatgt, taaattactgcactcc,
ctctggttcatgccgc, ggaggcttaggcaaag, agactcaatatgccct, gggccctgagaggccg,
attacttcacgtgaaa, gcgcttggaaccctgg, gggcccagctattttg, ataatctcctgaacct,
agaccccctggcttgt, cacacaatgaaccaag, ccccgaaaaacagaaa, cattgtaatgtccact,
tgataagacccaaatt, tagtatattatagccc, aaaccgaagaaaattt, agtttcagttcgccca,
gcaaggtttttttatc, catgtttttagagtac, acccgcggtgccttcc, catattatgcatcatc,
tcgttgggggggggat, cctcggttttctgctt, tggctatggaaccacc, cctgtgactcctaaaa,
gtgcatttagggtgat, gcactggacatttagg, agtccagttgagaatc, ctcactaagtttggaa,
gtgatgggcaagcaag, aacccataaaaaaagg, tgatgcgagattgcgc, ctgccccccctctctt,
gttatgatgctttgga, tacacgtcctcttaaa, gtatgctgtcctccca, tttaaaactaactaag,
aatatcttgtcctatt, acgtgaccgtttggac, agttagacttgcagcc, gagcataagtgaaagg,
catgttagcgaattaa, gccggagcagaactat, cccataaaaaaaggcc, ataggatgagttctaa,
ctctgcggaaggaccc, gcctgatgacatggcc, tcagtgctctcttagt, gatgaaagtgctcttc,
ccatacaggctgaagc, ttctogtaatctactc, gttcgtaacccctctg, catccccccacaggga,
ccagacgacagcccaa, tcagaggaattatgca, gaacatggggggggca, agtgcaacactttaac,
gacgcagtcttaaaaa, ggcagagagcggaccc, tcctgccaaaggattt, atcacctaggctgata,
ggctcacatggttaaa, actacctgcacatgta, atataccaggctttta, cagcggtgcggttggt,
agatcaggccacttta, atgcaagaggaacgtg, gcacacttataattga, aagggtgaaccatttt,
ttctaaaggcttagct, attggttcttataagc, gaaaacgtccagcctt, agtaaccgcatatgtt,
gaggcaggggggggtg, ggtgaacaaatcttct, aagaggaaagaggcgt, agggatggagccgagg,
ggtgttttttttattg, ctgcccttagttcagc, tttacgctgtttgtgt, ggtggacaattagcgc,
tcttctgttgtgacaa, agtaaccgcagtggga, cttgtccacctaccat, aagagagctctttaac,
gcccttttaagtggaa, attatgaacttagctg, catgttccagccctcg, aaagcaggggtttcat,
caagacagtttagtgt, catctgagataaattg, gggggggagtttctga, cactgggttccccaga,
tgtgttcttaacgaaa, tgaggccagtagatgg, ttggcataagccctat, ccgaaaaggaaactat,
ctaggtcaagaggtcg, aaaaactgatgtatcc, gtggcttaggcgggtg, tcgattgaaacccaaa,
ataagaactaggaagc, ggttaggggaaagttc, gaacgagcttcgctgt, agaaagatcttgttgc,
aaacgacaagtccagg, tcgttgagcacggggg, aggaggcttaggcttg, cgaagaaattcagcgg,
agctaccttttttggc, ggaggtgccggtgtga, tatgtttcacacccaa, tggtatgaacagattg,
agctggaatcttttct, tcaccggggacactct, aaaaacatgtgtgggg, tgcagaagaagttagc,
tcaagatgggggggca, gagttcagatactggg, ttgtagttgtcctata, aattggtttagtactt,
acttcccgtctgagaa, acccccgttgtccctt, aatagactcaggtggg, gggggcacccccagta,
acatactatcatgaaa, atttaatcggttgtct, ctgcttggatctgtct, cattcatttctacgta,
gggggccctgggatac, agaaggtaaaccagca, tcgtcaccccccaaca, aaagatcatttccgga,
ccctttgtatgtgggt, gtttagataagaggtt, acagctttgcttcttg, cagttgcttagtgtcg,
caaggtcagattagcc, ccttctgtgcttgccg, ggtggtaggactcagt, ccgagcattatggcag,
cgagctgggttcagag, caacccttgtgaatgg, agttgtgaatgcttat, acccaccgatgtgtca,
ctgaaacatgttatgg, gccccgatagaacatc, ccggcccgtcgccccg, actgagtctcggtcgc,
cacataggagcctgct, ccttcttaattccccg, atagtaagaaaccact, agtacggtgattttaa,
ggatagctctcagtgc, ccttctagactgagtt, taccaatcagcaggct, ggtgaggtatagtgtg,
ctgcaccttgacctcg, gtaaccgcttacccag, cgcctgccgccactca, cagatatgcgggatgc,
ctcaccgtagtccatt, attgtgagccaacgtg, gtacattgggtctctt, tctagctttgaaagta,
gttattattatgcaac, ccacttatagataatt, ttagagacgacaagag, agcacaatgtatgccc,
atgatgcaatgctgtc, ccgtgctttccggggc, gctcttcgaggtgcta, cttaggaccatagttg,
caccagagggcaaact, tagaattgtccccctc, attatagcctcgctgg, tgttctaaagataccc,
accccattggtgtttg, gcatctgatgaaacgg, cgtcccccaagaaaaa, tattcttcgtcattct,
tctacaccgcagtaac, cccacgatgaacgaaa, tatcggttttagaagt, atgcccggcggcattt,
accggttttcagggct, tggctgactaatgtgt, ccctggggccccttta, aattgacacttgatct,
agtccgggggaaatgc, gcaagagtatgcttga, gctcaaaaatgatacg, cctcaccgtagtccat,
tocacgcatcttaaac, ttttcctatgggacag, tacgtttctatttaat, cgatgggggttgagtc,
acggctgtggtgctac, caggtgataagcaact, ccgggaagagtcgctc, gcaactttgggtgacc,
cttatttattgtaacc, tttaatggagagcgtc, cacggttaaacccctt, gctaaacactattagc,
tgggtgaggggggggc, caaatagctcaatttg, tactgtggagcaggcc, gaccagtgggtgaatt,
gaccgatgaaaggagc, atattagcttccctag, gcctccccggccttgc, attggttggtcagtgg,
ggccatgcacattttg, agaggccccccccact, tttcgggggggggact, cttgctggccgcctac,
tcgctgcgtgaattgt, tgcacagctcgcttct, tttcctattgcctgcg, gatttgcgcagtgtac,
aagtaatctatggaag, ctagcagccctgctgc, cctgtaacattgcatc, gttttcaccaggcaaa,
ggggccctgggatact, tttcagacgccttgcg, cgtgggtatggtcccc, cttagctttttttggc,
tcgacgcctttttttt, taatcggaaactaata, taagagagatatgttt, tgggctacagccttga,
acctcatcgtcagtga, tggttgtcactgataa, cacgacagactctggt, gtgctactgtccacaa,
gacagctttgcacaat, tcaccctccgcacccg, ccatctaggacgtgag, atcaggtagagaatag,
agactctattgtgaca, cccagtacagtaaaaa, tgtccaaactctgaat, tacttaacgactatac,
cttatcacagttccac, cttaccagggatcctg, cttgaaccccccccaa, ctgggaacggatcctg,
atattgggggggggga, cctaaaaaaatgcagt, catagcaaaatgttcc, atccatctaggccggg,
gcacccgtcttgccat, atcccccctcccttac, gcctgatgactatcca, agctcaggatcttcgc,
gcctataatatggata, tgcgggtttccattgc, tccgggggaaatgcca, aaacaatactcccccg,
taacctatgtctgtac, tttgcatgaagttgta, aaactattgcgattga, aatgttgaagttgtag,
ggttggaatgtgtggg, gggcagctcgtgctta, acattgcgtctctgcc, cgcgtgtggaacgtcc,
aaccaagatttcgcca, tcgaaatcatgctttc, gcttctggtgactcta, caactggtgcgttatg,
gtgttccgcggccgtc, cttaggcgggtgtatc, cacttatcttctgttc, tccacaacaagctacc,
tgcaaacaggggagta, tggctcaggatttttc, ctattggagccattct, tgctcggaaggcttag,
catgcattgcattcac, ttagctccttttatta, aatgggtggtcctctt, aattttatccgaatat,
tcaacccctttttttg, actgtgggggtctccg, gtgttctacccagtga, tccctaccctagggac,
ggtgagtctgggatta, aacagcagcggtgagg, tcatcacttggtttgg, cgcccctctaattttg,
cgttatccgtatcaaa, ttaccttgtcacatgg, actcctgtatgtacca, cacgtctccatatttt,
ggaactgcggaggcct, aatctcgtgaaccctg, tcctcccctagttcac, cgctattttttttcca,
catctatgaggtccac, gggttgattgatgtag, taaacgtaagtaatag, taacacccaactgtaa,
ggatactagtttgtct, catatggcatggccag, cagaacggcttcatca, cttaatggtcagggac,
tctcacccactggtag, tgtaacatgtgtgtca, ggccctttggcttgtg, cagcgtttgtttaacc,
ctacaactgcccatct, aggtggccgtgctttc, actgttgatgtattaa, gctctgtaaccgctta,
acaagttogtaagatc, aatcggttttcaaatc, atagattttatcccct, gaggtgtagatagctg,
gactgtgacgtgtgca, atatcaagctaagcta, aggtcagtgggtgctt, ctgccctccaggtccg,
ggtacactaaagtgtc, acagcagcggtgaggt, actattagatgctcaa, aagtttgtggaacctt,
gactttccccttcaag, cacgaagtccaaatca, cccgggagtagggcgc, actaaagatctacttc,
ataggatggtgtgaga, agaataacttatgttg, gagatctccttgtaga, actatattggccagta,
atgcgggttctggaca, ggcgctcgagtacgag, aggcggataaccttag, tatgtgtccataactt,
gcggcaggtctttttt, gagtgttcagtagtgg, gactatgaccatgaaa, tgatacattccctgca,
acctgaggagaccatt, tttgcaagcttgtctc, agagaccatagttaga, gggggggagcagaagt,
ctttataatgatcccc, ttgtctgcggctgctg, gtaacagtgggtcctt, ttcaaaaaaaaggctc,
gaggggggggggacca, tctggtgttccctgat, atgcaaaaaggtctaa, tggggcttatttaatc,
aaggacgcaagtggag, ggtggccaaccaggca, ccacacgggaccaaca, cgcctgcccctagggg,
ttctgcccactttcac, tgggcagtcatggata, gatgcactccacgttg, caagtcttactgctag,
ctgtatagtattcgtt, aaggcctgtttagagg, tgtctacataacctgg, gggcaaaaaaagccac,
cctatcttttcctaag, ccggggtctcgctttg, agatctaattaaagcg, cttcgtaaacctaagc,
tgaggagcattgctgg, gttacccgcacatctt, ttacgactagcctggg, agttcagtcttcctat,
gtttacccccccccga, atcccctttaacgtga, agtgtaaaaaaagtgc, gtgtataggtttttta,
cctgccgtgccggatc, ctattataaacgcaaa, ccatgaatgcatgtcc, tgcgtcatgttcaggg,
aatcttagtacgaata, tacaggtggggggatg, gtacgggcggatgact, aggtccctggacacga,
gagcttattcccagga, ggatccactttccggt, ctggttaacacagtta, tattataacgatcatt,
cttgcggacttaaccc, aacgacacttcccttg, atgacatcgcctctgt, aacgttttaattgggc,
cgctggcagggttcac, cagttttttttggtgc, ctagcaacatgataaa, cgcccaggggggggcc,
tttatcacctgattcc, gccaaccctcgcttct, ctgtcaaatagttatc, ggttttttgtcattct,
catcttgaacatgctt, taatacagacttcttt, ggggcaggtgccggaa, attaggtcttactgtg,
tcacggcgcagtcttt, gtaatgatgttgggga, ttgaaaaaaagcctct, tgccgtagtccgggtg,
tgttatcatctgggcc, ggacacgaaagaaaag, acagtgccagtgcgac, agggatcgtctgggtg,
ccccgagtgacctcgg, aacgccgtcccccccg, gggtcttatttcaaaa, ataccctaggtcaacc,
gggaccttcgggcagc, gactgcattggtggga, cctaataacctatttg, actcgggcgctctgat,
gtggccgtagcttagt, aaacgtactgtgtcac, caagacatcgtctgta, gtcctgaagagacaac,
caagagttatgtcctt, gctaagaagaggggct, ctgcgcgtggcgctta, atgcatatcatggtgg,
caatagaactctgtag, catcaatttaccttag, ttcatgccaactgatg, ttaagatccttgcaca,
tgaatcgtagtcctag, tctccttcacgcttaa, tctttagacacattgc, tccacacgtgtgcatc,
tgcagttatccggtag, gcacgggctcttttcc, ccgccccggtctcccc, gtgggtatggtccccg,
cccagttctactaacc, acagcagctagttgag, gagacatgtctaacat, cccctactcaaattct,
atggttaaaccccata, ttgtgttggtgaaaca, ctgaagtttaggttat, gacaagagtcacgcac,
gccctttttttaacca, gcatcaagctccgatg, gatcagcacccagagc, ggtcttcagttcctgt,
tgagtcactgtaaacc, gcggtgtcattcccct, tttcatttagcttctc, gctctagacttaaaaa,
tactaggcagcaggac, ttttgggagtgacggt, ccttgttcctggttga, gggaaaacttaaagcc,
tgcttacccctaagtc, ctacatatcttgttaa, aagaagtggggtgcat, tgacccatataccctg,
cctatgaaagacaaac, gtgacacttttttccc, ggtaccactgacggtc, cttgtcactaaggcta,
gggctgggcgcgctcg, gcctcgggttgtatag, tgcactcaccactctt, gctatgccggccagtt,
aggcttaggcgggtgt, ggtcggtccgcatgca, agttgaggccactgat, ttctgagaaaaaatgg,
tcattataacaagctg, gcctcgagaatcatga, aacccctgtgtttgca, cctgctcgcctctctg,
agagagctgaataagt, tttctggttgatagcc, caaaattcaccagcgc, aggtatcttctatcca,
gtcgggggggggcatt, atcatgtactcacata, ctactcagcttaggca, acatagagtggtgggg,
gattggaatgaatctc, caccatgcaatgatca, atgggcgcccggctaa, tttttagggggggggt,
agccttttttttggcg, gcagatattccttagg, gttacctaaatcatct, gactgtagaccaaaac,
ctatcactgatcttaa, gaagttatattatgtc, ttggaaggaacgtgca, tagctaagagccaacc,
gacctcacggggagaa, ctctcgcacagcaata, ctcgtaatctactctt, gtgaaatcaaccgctt,
accgattagaatgaca, ggaaccccttaggtca, ttggcttaagcagagc, ctgcgctgtgccaggc,
tgatatttcggtattt, gataaaagttacctta, gcccggaatccccaag, ccctactttgtgtatg,
ctggtatggtagtgaa, caccaaagcggtgagg, tcggggatcctcagag, acgaagcccattttct,
ccattcttcatcaggc, tacttatgagggccta, gcttatataacccatt, actcaaatctgtaagc,
tgccggtttgtactga, attaatcgtccatgga, agaaggctttagagcc, ggtctctgtaccttcc,
gcccgaggcgccttcc, acccttagcagcacaa, acaaggtgtttttgtc, cactagtttgttatct,
tcccgcttaggctgga, cctttttttggcaacg, agcatcaccttaaatt, cggccatctctacaaa,
cttagcctaggtgcgt, actgtggggggggaga, ctcttagtacccaagt, tcagagtgagatcctc,
ctaaaaaaatagggct, aatatgtttggtcaag, ggggacttactctcaa, ggggcacttaggagac,
cctgccgcagtcccct, gtaaaaaactgttgac, ttgctttttttgggcg, cccgtcaacagtgcca,
gaaggcatcccctcct, ctgtatgcccatcccc, tagaatccaggaccaa, ctcagcttagagctat,
atccagatatcataat, aatagatcgtttttta, gtttggcgtgcactat, gtaggttagaggtcca,
gctcttccaacttggt, aatatacaccgggcct, aatatgggggggatgt, gcgttttttttgttaa,
gtgtgccgggcgaagc, ataggtcaggatttga, agctctcacgcccacg, ttagggtgatcttata,
aatttctgtcatatcc, cccatacccacataat, aaatgactggagtggg, tagggggggacgggtg,
tctattttgtcttccg, tccaaaaaaaacgttt, ttcccgaattcagtgt, attgccctacctcagg,
atctatgtcagtccgg, gggtaaaccttataca, tattaaagctagagtc, ggcatataataattct,
acagcccctgtcgaca, aacgaaaaaaaaaggg, tgtcttattagctctg, acaaacgttatccttg,
ttggtcagcgactcaa, gacttgggagccagct, gtgtttactcgagaac, tgtgacttattgtctc,
atgtacttccctacag, cctgtgctccacggaa, taagtgccaggacttc, agccctactttcaagg,
agaccccccccatact, agagaacaagtttggg, ccatctcagcggctct, gggcgccccccgctat,
ggcattcttttcccgt, ccatttagatacgccc, acacagtttcagttcg, ttgactgggtccctat,
accccaccctaaggta, gttgcttatgataaac, gggacacagattaagc, ccatttatttttcgtc,
aaacttatcggaggac, caagcatggcgctggg, gtgacaaatcatgaga, gtgaataatggtgtct,
acaaaagaggaccccc, actctcttcgtttata, ccacggggtttcttcc, attgcgccggcgcagt,
cagtgacacagcccat, ggtacgggcggatgac, ggctgggcgcgctcgg, agcatgttatggacca,
ggggactcggttttta, tccctttttttgtagc, agggaacgtcatcaga, ggagtagctaccacgt,
atcacgtctgcacaag, gcacaatgtgcgctag, ctaccggccttgcctg, cgcaaaaaaaatccat,
ggcgaagtcaccttga, agctaacttgttttac, accaagttagcccctg, gcaattggcagcaaaa,
cctaccccccattaca, tagcccagttgtgtat, ccgaaaaaaaagttac, tcaagtcctccccacg,
ggtgcgtctgccccac, agtgttactccgatac, acgcacccatcctaac, tatcccacaaatttgc,
gctctatagatttctt, gaggccctagagatgc, ggatgcaaacactgcg, catttattcgcatttt,
acttgattgtcagctt, ctgacggtcacactgt, ccgggatcttggggct, tctagactgatccact,
catagggaccctggaa, gccacatggccacttg, ttgggcccccaggacg, agccaactccaactga,
atgtctacagcctata, aatctatgccccagct, ataatggacttttagg, cgggcgtgggattacc,
aaacgtcatttaataa, gggtgtctccctgtaa, ccaatgtgggacagcc, catccctatgctgctc,
ttggtcgtatatatat, tctaatgcccaaaatt, ctagtaataatatccc, tggctccaggcaagaa,
atggtatgaagttgag, gtctttgtctcgctga, ttaatatgaaacggta, aaagttgctctgccgg,
tttgcactattcctaa, tgaatcccccccccct, aagccatcttctccgg, gaaattaagtaattcg,
gtgagtacctaaaaca, cacctaagctgtgact, aggatacaaaaaaagc, ctctgtgtgctgcact,
actatggtccatgcca, atgttcctgctattac, tcatggagggtgcaac, gtacggtacaccacgg,
ttgagggcattaagca, ccgccttagctggtat, gtatagccagaattgc, gacaattacgaaggaa,
tgtcttacagcattag, gggtttttttatggat, actcaatctgagaaac, aaacaggtggtcagac,
tttgatcgtaacttct, gatatagatcaataac, gcagaaccaaaacagt, tttcgaaaaaaaaatg,
gtatggcatggcacat, aacctaaaagtgttat, attccgtctgcgaaaa, taggctcagcttaagc,
gggaagtctaagtccg, gcttgctcacggtgcg, ctctgtatagaagcgt, gttagcccctgaccaa,
cttcccactggcggcg, tcaccacattacaagt, taggacaggattgagt, tttaaacgatatggtc,
gaatatatttccacta, cagggttagacctctt, caggtcagggatcgtc, gcttgcgagttttggt,
tttacaggcaactatt, gttgtttcgttatctg, ctttggttctttatcg, cttcggccccctctgg,
agactgccttagagtg, ttatcggaggacagaa, cgctagcgctataact, cacgttaccactttcc,
aggggccggactctgc, caggggggggtggatc, cacagctaaggtcacg, tatagtatagaatatg,
ggatggctgaattgtc, actctcccaaacctta, agtattcctgggcccc, cctgaggcactaccct,
aagaactgattaatgc, tatgctccatcccggg, ggagagcgtcttctta, gtgaatagtgcctaaa,
ccgtaggcagagcttg, ttttgtcccaactgga, actatcaacaaccegg, ggtactttccttagtc,
cggaccttcctctccg, atctaagtgcctcttg, acccaccttttagagg, aagtcaagtagaccct,
tgttttttttacgcaa, agaacacgaaggtgga, tacccaaattttttat, agaccatttcttgtgc,
gggccgcgttgcggcg, ttggctctggttgggc, tttaagttacatttcg, tgctatcctcgctgat,
gcacgtcccatacccc, caggctggagttgcga, ttctagagatccaagg, gctaaaacctcctggt,
tttaggggggggtaga, ttagtttgttaggcat, taatgtaaaggaaaga, gggttcatctctctat,
ggtaattcaggctaag, tgagcgtagtgactaa, aatgaatataagtgcc, ggtccatgtcacaatg,
cggtgccttcctccgc, gcactcgggtgtctcc, cagctcggttaatacg, ggaacttgtttggtga,
ttgtttttcagccgga, gctgttcctattgaac, ctagggcggttttatt, agccctaaaaaaaggg,
ctgccattgagtcgcc, ttcaatgtccgtgagc, tggtgtcatagtgatg, tctcaggcccttagtc,
ctggtgcctataatac, agttattcccccccaa, tgactgtctgattaat, attgatatgaggcatg,
cgcagcccctacgggg, cctaggggcctcgcag, ttgcggtgttttctgc, atccaataggtgaata,
aatgagtttaggacta, gccgtgcccgtgcaca, tgggtgaagacggatg, actgggttccccagac,
ggccctggtaggttct, gacgttaagtcctccc, atagtaaaagggtgag, ccgaaaaaaaagacct,
tataccaccagtgccg, ggggcctcgtcaggca, tgtagaacgtagacta, gagggttggtcatgaa,
aactacatccactgag, cgcagtctctgtatag, agctaagtgactcctt, atgctctggtctagcc,
ttgacgaagcagttat, tttagcaggatacctg, tgattatgatggacta, acaactaaatctggtt,
agttgtttactgtggg, tccacatagggaccct, tcaaacacaacgaaaa, aattctctattttccg,
ccttttttttagagcc, atggctagacctcgtc, cttagagcacatatag, ctactcaatctcacta,
accaaagtcttactaa, cagaatagtggagtga, agacggagtctgacgc, accctgtgttatgaat,
cctccaaggggggggt, tcccagtgtgagatga, accctacctgtaaaca, gtaaaaccaatcttgt,
gtgtgaggggggagcg, tgccgttctgaatttt, gagacgaggttttacg, tagactgtgcacttcg,
cacttaaaccggtttg, tacaccgcatttttcc, acaaaccttcttacta, aattaggtgaatagat,
agcgcattaccgtgtg, atcatgtcagcgattg, tcctccgccagcactc, ctaagggaggtcatta,
ccacacaggatgttaa, aagacggatgcgggtt, tgaccccccccccgcg, tgtgccgctgcaacct,
tgaccctccgggaggg, gtccagccagtggtta, ggtttgtagcacagta, ccctgggaattgactc,
atggttaagcttactt, ggtcagttatatcatt, aagacaaggaagtgtc, atattctgactaagga,
tatcactaaatctgcc, atcgaatcaatgaaat, gttattcccccccaaa, ttctgtaggcccaacc,
tggcaagtagctacaa, ttctctcgtactcttc, ccctagatggtccacg, ttagcctgcgtggttg,
acttgtctatgccttt, ggtccaccatgtggtc, ccttagggtttgttat, tatcctttccacgttt,
ggtaatcctcttctga, atgggcctgtcagtag, tctcgctttgctgctt, tgattaaaaaaacggg,
atgtgactgcaccttg, tcccacctacgttttt, gccgcggggtactctg, caggtccacccgcccc,
taactaggccgccgac, ttcgtgggaaaagatg, gctttaagcctcgctg, attgcatgtaggggtg,
tggagaacaacgggca, ctggtaggggggctgg, ggtgggcgctcttagt, aatatcatattgtcca,
caacattttttagggg, tgggactacatgcgtt, ggagggctgtggtgcg, gtatgatcatgggttt,
cattttcctcgaagtg, aagaaggagaacgcat, cttaaaaaaagtggag, gtgagtcgaggtctcg,
gaatgtatggatccac, aatacgcgttcattag, aagatttcggagttgt, ggttgtaaggaggcag,
gcaattgggggggtga, agacagaacagcagcc, gttttttttacaccct, cattggagatgctggt,
gaagttagacccaatg, cggggggggacttta , gaggggggggacagaa, gcttttttttggtgac,
tcagcacgtagaaatt, agaacccttgctactg, cagttgaaggctgatg, tatccaatattgctaa,
ctgggcttattgccct, ggaagactcactcaga, cataaaaccctaatta, ggactattatggggtg,
attcggcaaaattaac, atgggattttttgtca, tgctactatggttcaa, tataattttccgtata,
atgcactccacgttga, acggattcagagaggc, acggcggagaaccttg, acgaaatactgtgcac,
gggatagctctcagtg, actcgggggggggatg, aataaaccagacgaaa, tttcatgtatgcttac,
ggcttcctaattaggg, tccagactctgactca, tttagctaaagagtca, ttagtagcgatgctgt,
atagtccaagtagcaa, accgcatttattaaat, atggtctggatgactt, cctctaaaaaaagtgc,
ctctactttaggaatt, taattaagtggagtat, ccaataagttggtcta, gttcatcccgcaccag,
tcggggtgtctagaca, gcacagctcgcttctg, gcatgtaaacttagac, agaccaaatatcagta,
tctccggtttcatcta, cattttaggtactttc, atggtattgactgtag, ttccctagcaaaaccc,
cactacttttttttac, gttaggttatggcttc, gatgcagaagtaatcc, caaaacacaacgatgc,
tcaacggctgggggga, ttcccaccctatattt, catagtcttgaaaggc, gaccctgacatcagtg,
gcggcttagcgccgcg, catgtctggttaatat, agatatcaaaccactg, gcagactagagctatc,
gtcttaaaattgcagc, gttccagccctcgatt, cgatattctgatagct, ccttgcagagtaagtt,
cagggaagcgtttgaa, attatgtcatgtacca, acacctccagatgtac, cagcgtatcccatgcc,
tcacggggccacaggt, ctcaagcaaggaacgg, gcctttttttacctac, attgtggcacgagcct,
gttaaaaaaaagccgg, gtcttacccaactagc, caaagcgccttggtca, cttgccgcgcgccggg,
tatggattctgcgttt, tttagtgggccactta, tggcactatgactggc, atccgttgcatggaag,
gctaaaaaaaagccat, ctgctatcctcgctga, cctgtagtgtagtgag, caatgtgttgaatctc,
ccgcgggcgctttcca, cgcccccccccgaggc, catatgcttcccccca, ataagacacataatac,
caagctccgatgaata, gaagacaagtaaaagg, cattcccccccctctg, acttctcaccatttcg,
ggtaaacaaacaacac, ggggggtgtaagaggc, agccccccctgtgaca, agagtctcatgtaagc,
tgtaggatttgacaga, ttaccatgtgggcgag, gtctaattttaggctg, tatcttaagaagcagg,
ccacttagaggaaacc, gtagggagcatgctca, gcccctaagggggcag, cagtgttccgtgtcat,
ttacacggagcactag, taagtttccttttcga, gtaattttctgagggc, gccactttttttatca,
tcaattttcgtcttaa, ggcttcacaactgcta, ttttaggggggggctg, tgctcacagttaccgc,
gaggggcttgagttta, tcctgcgggtttccat, taaaacgaccgatgaa, tcgaagctctcacgcc,
tttcattggcaagcta, aagctctcacgcccac, tcggtgatatttaatt, cggtttttttaaggct,
gtcctagctgtcacaa, cataggcagcagccac, tgtcgctcaaataaga, aaccaaagtgcagaca,
gatttccggttctctt, tcttaatccggagatc, ggggttgagcggaggc, cctactcaccctctgg,
aaagagcttgcggaca, gctgtatttgccagag, agaaacagacattcgc, cgttttatgacaatat,
ctcctgccttaatgtc, aatcagctcgtggaca, gggatgcccttgccct, aggcgttgaggagcgg,
acttcaatttactcct, tctgtttcggggggag, gggtaccctcattctg, acttagatcactaatg,
gtacaaaaaaaagggt, gtggatatggtaagct, ctaaaaagtggagtgt, aggctcgcacccattc,
tgctcacttactgaca, cacccacaactccacg, aatggtgcacttatct, gtaggagccagttttt,
catgcccccgaaaaaa, ccactgcattgccgga, caaaacttatcggagg, aaactacatccactga,
caacacagttagactg, aggcgcacaatacgcc, actttcacaccttaac, gtgctaggaggacttc,
tgtagagtcttagctg, ttggtagcggccgctt, gtgacattttttttgc, ccaatcacgtattttc,
atccaagctggacaac, ctctgagtgaagagct, ctaccacgctttctat, agttttggggggtcta,
gaaccccccccaaaca, catctattgtgggtag, atttgagtacggaact, gtgggaggggggggtg,
taaattagatactgga, acgttggccacttact, ggcaatataggcgtgc, atagctaaacatactc,
acgtttgcttctgctg, gtatcccgaacactta, catcaaggatgttagg, aaaacgtcatttaata,
agaaacacagcgttag, ttacacaatagacagg, acttgagcatgggtgc, ggtggactgaaacaac,
cttccttcgggctccg, actgtgttgaccacaa, gcccggatcaggaatg, accccgaggtatcaag,
gctaccggccttgcct, gatggtcggctaatat, ctcctgatgcagtctt, ggtggggtaactgctt,
agctgtgtagcccggg, ggtatgttttttttgc, ctttgctccttcgttg, cctcgatttggctggg,
gtgaggtggcttaaag, acgtatagacatacgt, cgggtctccgactgtt, cggcggaccttcctct,
ggaggcttaggtaggg, gctaggcggggcttcg, gtttattcgtaattat, agtgttcgttagatca,
caggatcagcacgggg, tcaaagagcacggggt, ccagaggttctagcga, agggatatacataact,
tgtgtacccatagcca, tccacttcccgtgttt, tgacacttgatctaat, cacccaaagaacccag,
aatcaccagataatca, gttccttttttacgct, aagacgcagtcttaaa, gcttcgctgagcctgt,
tagagcaaatctggca, actagtctgtgagtgg, taggttcattctagaa, gtctacacagcgcagg,
aaattgggagtatgca, ctgacggttttatagg, agctgactgggatact, agcaactcaggccaac,
gaagtgtgcgtctgca, ctcttaggagggcaga, tgggatactcaggcat, gaggaaacttaggtcc,
agttctccgccctcat, gcaaatatcagtgaag, caaccacaaaaggtgg, ttagtttttttttcgt,
cctaggtggaggccta, ggatgacctcgtggtc, acagctcggggggggg, gaggtctgaaccctac,
ccgtgcgctcccagcc, atgtggttctccagca, gaaacccccctgccag, ctgatgcttgcgagca,
tgtggatatggtaagc, gcctatgagaatttct, acttgtgccattgtat, gccttaccactgttta,
aactgctgcacgaaat, gagatagcttagaatg, aaccttcacgatctct, catatacccaaacgac,
agacctcgtttataaa, ggttagggggattagg, ttgttctcccgcaaag, tccgggtgctcttgtc,
tacagaactgtccacg, ggtgagcttagatcag, gtgcaccctgtgtaag, ctcgccttgggggccg,
tcccccattttcatac, agctcgaaggaagccc, gtcacaaaactcagca, ttacccatccacatgc,
ggacacattaccaagt, actgttgggccagtgg, ccaaaaccccctgtta, agtcggttttttttag,
atatatgatctggtgg, actgataaatgtgaca, gccttttttttggcgg, aatttctgaattcggc,
ctctgtcccggggacc, tttatctcggaattct, acgtgggggtctccct, tgatgtggtcagcaag,
tgacaagtaaaaagga, ttgtcatcttatctaa, gtactcatgagcccag, attatgcactttattc,
ttttgtcagtactctt, gtcctattagaaagct, taaagtaatacgtaaa, accccgtgctctctta,
ctcgagaggtccagaa, agaacactccgcccct, gttccccagccgagcc, gcagcttccttataag,
gtgacgatacgcgagc, taaccattggataaac, gactatgggcgcccgg, cttttcgccactgcac,
gctctgtaccttaggg, agtggggggggcactt, aacctgggtgaagaga, catttgctcctttgag,
tgtgctggggaggtct, ctaatgcacgcagaat, tagttcaaaaatttcg, ggatcaaaaaaaaccg,
gacggttttttttaat, tcagaggataatctgc, actggccaatttggcc, caaggctcggaaggga,
ttatagcctcgctggc, ctgaaccatagctgta, catttcaatgtcggtg, gtatgaaggaaaattg,
tcgcaggagtagccag, ggaagggcgaactcta, cccccccaggccatag, cccaccgatgtgtcac,
ttaagagttatcacta, tcagtgatcatggatc, ggagtaaactacagca, gtgagagagtgggggg,
gcccagctattttgct, acgttccaccacccct, tcgatttctaaactta, atggctgactaatgtg,
gggcctttggcccggt, cgttttgtccggagaa, tttttatgctctatcc, aaatgaggaacagcac,
cgtcttggttttttgt, tgtcctgtatgacata, tactccccttatcgca, ctaataccttgttact,
acctgggtgtggttaa, catagcaggaaagatc, gggtagcctttacagc, ggtctgtgtcacgcgg,
ttcccatgtagtacca, attaatggggggggag, attacaaaaaaacggt, acctgggtgcctaatt,
ttgtcacatcatgcaa, gctattgccagtctga, tatacctaaccttgga, gccctgctgactgcat,
acatgccagtccaagg, cctgcaaaacttatcg, ctcagccttctttaag, gtagtttacctagttg,
ataagccacctatgca, cagttctgagccacaa, gtgtggaacgtccgtc, cactaaatgtaagagg,
aggggtgtttgcagag, ttccaatacaacgtaa, cccgaattcagtgtct, ttggtgaccattagag,
atacttattctttggc, ggtcctctgtcactaa, cgctggacccacgggg, tacaccagttcacctc,
gtgaaaaaaaacgctc, gtaaccatgtcagcat, cacgaaggcccagtga, gcaacgtaggataagt,
tcaggtcagggatcgt, tgcccactaacaagtg, gcttcagaaattactc, tgcggtgttttctgcc,
ccctcaagtcagcatg, cagaacgagaaaaaag, ctcaagcccgtgaaat, caaaagcccctagccc,
tattggcacctctaaa, gcctgccgcccattgg, tctggggtgccacttc, cttgcgcaggggcccc,
gatactgacatagtat, ggcgggggggattctc, gtgattttttttagcg, cagttatgcaattccc,
gtcaactatgagcatt, tatcctgcaagattta, tgcccccgccgcctcg, taaatgttgtccacaa,
gttttaattggggggg, cagagagtaggtaacc, cttatatgtcaactaa, ggtactcagctgtatg,
cctgacttgttttgta, gcttaaggtgtaagac, tcagttcgcccaagac, tgtatgtcatagcatc,
aggtgttgtagtctag, gtctactgtcacaagt, tctattgtaatgtctg, tgttagtaagtcagta,
aaagggtaagataacc, tctctccaaaaccgtg, ggggagataatgatgg, ggcttggggcgcaaca,
acaagtgtcaggtctc, tcagaacgtttccctc, atgttttactggctgt, gcccccccccgactag,
ccaagatcccggcatc, gctgtaagttaagttg, tccatcggggatcctc, gggggggggagtttct,
atgaaaaaaatcgtca, tgtgtccccccccctt, atgtgcagccttagac, acaatggctcgacctt,
tcactgtgttatcgag, gggtagaggaatttaa, ttctatgcatgagtat, aatgatggccccgtgt,
aacagagtcggttttt, cggtctcttcagtttt, taccttagcttcgtgg, ctaccactcacctttg,
gaaatttacacgaaaa, tttctccccccctaaa, gcagcgctgggttctt, gtagaatcataatgaa,
gatttgtcagatgaat, ccaaagcgccttggtc, cagaattcttacgggc, ggccttacattcctgt,
actggtctgtgtcacg, cagctgctatgatgac, cacgttaaatcaacag, gctgaagggtagcctg,
ccttatgaagccaatg, tttgggggggcaaaga, cacggcttaaagctat, tttggcgtcagttata,
ttatcatggtggcatg, tgcttttgccggctta, tgttaggggcttagga, caagctgtggtttgct,
cagatggtgtccttgc, actcgggtgtctccct, tagaaaacaagacctc, tacaaattctgtattg,
gaagaaactaacgtga, agccttatccatgccc, cgtgtgcgtgaggaga, ctgcgccccccctttt,
ctgcttagtacaagga, ttctccagccccacga, aaaaaatcatgaacgg, tatggttccccagccg,
ggaagggggggcccac, tcgatggcctttactt, cgtcacaattattcca, gaatttcgtggcctcc,
tctcgccttgggggcc, ggttatatttctatgc, cagaccctcatcctga, tatctttcccccccac,
catagtgctcccgtca, tgctcaagtgctacag, ttccattcttcgcata, tggtttagagttatcc,
agcccccttgaccagc, tacctcttgtgagact, gcttacttccacatta, tctgagaccacagacc,
tgtcttacttatgcta, ggctttccaggatgcg, gtgagatgctctgtta, cccggggcaccttgtg,
aaccagtgcaccggtt, aaagagcgtgtatagc, cagaaccctctctgag, atgggacaaggagacc,
ttggaaacttcttctt, actgacttcgtttcgt, ttccttcgacacttgg, gacttccgcgcagtct,
gttcagcctgtgccaa, caacgtaggataagtt, tttttatgggggggga, gctgagacgatgtaga,
caccactcccaacgtc, cgtacatatttgggcc, aaatgtaatggataac, tagttctcttttagag,
ttgacctcgcagcctc, ggtgcgttatgaagta, ggactcggggtgtcta, gtggtagctatacatg,
acaaaataccaaccat, agctttttttgccagt, atgcagcataccagta, gttacagactgagact,
actttggcctgtcttg, tacgccaagtgcccta, ccgaatataaatctgt, ttcccggccaggcttg,
acgatttataatacta, ccagcggcaggtcttt, ttcaatttttggagcg, ttacggggcagatgtg,
gtcatttttgtcgatt, ggcgagttaatacaca, gagacgctgccttgat, actaagtaaggtgctg,
cgaatttctgacctta, agtagcgcgaggccca, taagtaccgtgtatta, ggccaaaatgggggac,
gtagcgcgaggcccag, aaatttggtgtatggg, cagagtgcttaaagat, atcaaatttgtctgtt,
atagagggtacccctc, gccctaaaaaaaggga, cacactttgtgcacat, aactaccatttttttg,
tttaagggggggggtg, tgggcacttagctatg, tgtgggggacatggta, acgagttctgagatga,
gggctcgtaagtcatt, gcagtttgagattctt, cgctgaaccaggccta, tttgcaggctcaatcc,
cattgccggatgagga, gcagagcaagatcaga, atcatctgttggattc, attccaccttgaagct,
gatctcttcctcttac, ccatctatgccacgaa, ggagccccgctgtctt, ttgtctgagagatcct,
tgcactcagcacaagt, acagcaatacacggcc, tttttgagtaacccct, tatagagcctattatc,
gggtctcttgcgtttg, acacctcacatgggag, cgagcatatgatgatt, gcagtgacctctctca,
ggatgccgcatgggct, ggatactccccccact, catggtgcgcgcgtgt, cgtgtagaacgtagac,
tcctccaggccttagc, ttcccacctattagta, gattcgattttgacat, tagtgggtccttaggg,
agtcacatattgcagg, attcctcacaatgata, tggaggtctgactatg, gtctccctgccacacg,
ttattagcctgactgt, ttgggggggcttacag, tctaagacaagctaca, cgttttcaggactgta,
cccaacagattcagga, tagggccaaaaaaaac, cgatttataatactag, agtttttagggggaac,
tgaactctaatggtat, attaatcgaccataaa, cctgcgtagagatatg, ctctgttccatttccg,
cggctgggatcgaagc, gcgcaaaccaggccac, atcagactggtctctc, agacctattggccacc,
gcttactctaacagaa, gtcaattcttgcgtct, ctttggggggggaaca, aaccccagaaaatccg,
cctgtgccacccgtga, aggatgggtcggcctt, acggagtaaactacag, ttagcacaccaagcat,
agtgctgctctacctt, gacctactcctacaag, tcggtgaaaagccacc, tccttcatctaagggt,
ttttagactgtgacgt, ccactctagtttctcc, agcaaggcaaagtttc, ctgaatggttatttcc,
ctcttgtctggccctt, ggcacttacacatagc, atgtgcgcgggcgcct, aaatagcctctgatgg,
taggggggggagggtg, tccggtgcgcgggact, agactgctgttcccac, cagtgggtagttattg,
cagtgcgactgtgtca, aagtaagacttgctta, gtgaagacggatgcgg, ttgtagtgacacatga,
atgcatatgctactac, aatacccgcttaataa, tcgaacatgttgccgt, atttttagcgatgtag,
ctatcctttgttcctg, acgccccccatggacc, ctcttcacgtgtgttc, tgaacgagcttcgctg,
acaccgtgagcattgg, ggaacaatcacgtctt, tacccggcccagatcc, tacttagatcactaat,
tcaacctagggctgat, gctgtggttacagcgt, ttactgttacgagaag, cgctcggaggcacaag,
atgccttagtgtcagg, ggggaacattattgta, tccttagttttgagcc, gcctctggttcatgcc,
gacttcctcagtacta, ccataaaagtgaattc, aagtccggcaggcgcc, attgtagttaccattg,
gacgtgcaaaaaaaac, aaggggcttgtctagg, taaggtttattaagtt, tgagttaacaccacat,
attccgcgctccgcgc, agtgggttacccctgt, tcagggttgccaccgc, gcctagggtatgcttt,
attccttttgactcga, ctggctaggctttgtt, ggcagctggcctgacg, tgttcaagtcttccaa,
actcgtgtaacccaca, atgaactatttgacca, ctcattaaaggggaca, tggagttaccctgatc,
taggccgtccctccca, ctctccgacttgtcag, ggagacttgagggtcg, aatttaacaggggtag,
cttgcacaaacctggc, tgagcttgcgcatggt, gagttctcgcgtgatg, ctcctgaaggacaatt,
ggggactattatgggg, tctcagtgtttgtcgc, cccctggtagcccttc, agtactttaatctaaa,
gattctcctgattcgg, ctttcggctcatcagt, ggaaaaaacggggcat, ttaaggaacaccaaac,
gacggagtctgacgct, acgtgccacgatggtc, ataactatccccccca, agagcttttttttact,
cgactttttacaaagt, atctgagtcccagtct, gctcagagtgaagatg, cttagtcatggtcctg,
aggcgacaagtcatag, gcgcagaaattcagag, tccccccccaggccat, gcccctaggggcacca,
gtcattttttggcata, ggcccccccgagccat, agttcgcccaagacag, cggaagtgctgcgttg,
aaacgctgtagttttt, cgtaaagatactcttt, ggcttagtggcgagcg, ggaagactagggcggt,
ttatgctcaaggaccc, tccttccgtcttacac, ccgccttagcctccgc, cttgacttagtccttg,
agttgtataattgaac, cgtggttacacacgcc, gccgtcgtcctcgaga, tggtgggcgctcttag,
cccacttggatccagg, tttcttcactttccgg, aaattacgtaacatta, ggctatgccggccagt,
gggaccgggtttctgg, cagaccacttattgcc, aggaatccctcccagg, cccctgagtacgaatg,
agaaccctacccccca, agaaaaccttatagtg, tacgcttttttttgag, tgcgatgcatcttctt,
aatttttagtgtcctc, aagtgatatgggtttt, gcatactgaattcaaa, agttaactagtttcta,
ttaatgtcgcatctat, gttcattaggccccag, tacttatttgtcagta, tgcctgcattagattc,
gtgggcccttcacatt, atttatggggtgagct, aaaagtgttgacgttt, gcatggaggggatctg,
cgaaaaaaaagacttc, gcatcactcatacaat, cggggccacaggtgtc, tgggccaaattacttg,
ttttttaatcgcttta, caatactccactcatg, atcctcgctaacagga, gactaaaaaaaactgc,
gcactttttttgaatc, tgactttgacgagctt, tatgcaaaaaagcgta, cacagctcaagcaagg,
gggtcttaaaagaata, cataaaatccacttga, ctccatacccggggcc, ctaactatgtcctccc,
aaccagagagtaactt, gcctcggcgtcccaat, tttccggggcatcact, gtggcaattaggattt,
gaaaactgttggttcc, cttattctaggcttat, tggtgttgttattgcg, tcattgatccagcgaa,
catcaacagctaggcg, atggtatgggggggta, ggaccctcgccttgag, aagagtccccctaggg,
agtaaagctcaaccag, ttcaaccttattacca, tgcatcttgcgcgagc, gcaccatagtggggga,
gcatttacaagctact, ctggtttttttagaat, ggaccacaccaccccc, ttgatctggtgagcca,
gaagacctactagttg, cctaatatggattccc, gggcttaaaataagac, cctgaccttgactgaa,
gggttccgccccccgc, gctgtcagatacgctt, cactagacaaggagcc, ttgccccctcatgtgc,
gtggtcatgaggacca, acagtggaatacttat, gccttgcgttcgccca, ctatttaacagtgctt,
atggctggttcaaata, ctccttgtgtcagaag, ttcactgtgttatcga, cacctgccccacgccg,
cagcccaagactgtgt, aaggtccctaaaaaac, acttagtaaaatcccc, tcctgaggcaaatgac,
gtagcgagactgcctt, ccaaaaaaacgggtta, gtgtaacagtagaact, ctgctgaaactataat,
caaggtagctccattg, cttcccatgtagtacc, gtacatggccaaactc, ccggggggagacgatg,
atgctgtcccctttta, gggagctgaagtgttt, gagtcaatgttttgta, gccttttccaggtgat,
aaccaaaccctattta, acaattttttaggagc, attttaaacgctcaat, aggggataagaccttt,
tagaagcttccccccc, tacggtgattttaact, atagggggaggctcca, aactcgtgtaacccac,
cattacccagcagctg, ctgtcagtaaggcagt, gtccagcctgtgatat, cattcgcgtctgtgtg,
gagggagaatctgata, tgcagcccggggaaaa, tacttattaaacccag, tccaaatgccggagta,
acgcagagagggaagg, aatttccgtgaaacgc, gaagccgcggtacagc, acttgaccttgattag,
ctgattaaaaaaacgg, agcaactttggggtac, tgtgtttggcttagct, taggattaaatctgaa,
ttagacgatgggattt, ggctgacgtccgtaat, tttaaggggggggtgg, gcgatgctttctttct,
ttaagggggggggtgt, cagcacgagattatgg, ttattaggtagcgcaa, ttttagtcataggtct,
gctggctggatgccat, cttgccgcgcccgcgc, cacaactacctatacc, gatgatctctgatgcc,
ttatctctcctgactt, ccgttgtcccttagag, gatgtcttacatctct, ctcgggcgctctgatc,
gacattagcctaatgg, agaccccgccctcgcc, cgcccaactttgcctc, ttaggggggggctggg,
ctagtagttgttaggg, tacgacatactttaat, cattacaatcctaagc, tatggaacctaaatag,
tttctcagcaatacgg, tctgaccctgatgccc, ccggtctcttcagttt, cctaaactttgtacaa,
gcggctggacattgac, cagttagaaccacttc, atgaagaggcacaggt, taacgcggttaaaccc,
accccccggtgttgga, ttatggtacgtaccat, aaccattacctggcct, accgggattaagggta,
gtatgcgaaaaaaaag, gtatctccacatgtta, caagttcgtaagatct, atctggttaaaccctg,
ggcctatgagaatttc, gtccggctgctctgct, tcagcaatacggtagt, taagaccatctgaact,
atagcaagttcactag, tgtatgaagtacgtga, tgtcaactatgagcat, tatcgctttttttgca,
gtaggggctccacggc, acaatcatctccacag, tggagcataagacacg, cattcggccagtcctc,
gatctgtgtgctccct, atcagagcactcgttt, gttattagggggtgaa, gcaatcccccccccac,
ccggccccaagttgtg, gcaagggtttaggggc, acctacttttataagg, cgcctggggggggggc,
ttttgctgcgcagctg, ggacagctggctatga, gccctacaattcatct, gcgcgtggcgcttacg,
cattttttttatcacg, tatgcaaatgtaggtt, aaccaggtgcagactg, ctataaggatacttgg,
agacgcctttgcgggg, gaacaccttagctgtt, gggaacctaagattgg, caggcgtggtgtctac,
tgggactccctcttta, ccatatcctgtcctta, acagaagcgtgccccc, gctactaacccagttg,
ttgtattgatgaaggg, ttaagagatgcagtgc, cttctaagggtgaagg, ttatcatggggggtat,
aacactctcactcaca, agcagatcaacatttg, tatgaggaacttttta, aaatagcgaagtagac,
tcttataaggttatag, catgccgtttttttta, taactaaagaggcata, tcaacagcctttaata,
ctggacgagtgacctg, ccctgtatgcgcatcc, gaccctttttacttgg, agtgtgaagatattat,
tcttgcttggtgagtc, gtgtcttctttacatg, ttgtcgctcaaataag, cacccgcctgatgctg,
gccttaaccagtacct, ctcgaatgacttcttt, gcgtacaaaaaaaagg, gctgccagcgggcgct,
gatagttaagtaagca, agattgttcctgccta, ggcgctgcgagggcgg, ggcttccacacagacg,
ttagcgccgcgcagcc, aataacctgtaagcta, acgtggttaaacctca, gtgctgtcctaaagat,
gcggctctcttccctg, gtgtgaggggggtgta, tcactgtgctcgccgg, cttcatggcctcggtg,
tatattggccagtatg, gatagcttaaacctga, gccaaaaaaaatagac, gccttttaacccctgg,
aattttttttaccgag, gagtccttactcacta, tagtgaacgcaaagac, aagaggtccagaggta,
ctctcgcaaaaaaaat, ttgacaaatgcactct, ccttcccccacgaaaa, aattccttcccccacg,
ggggggataataacta, gttatccggtagaggt, cttagctactcagact, ttgtaggaggaaagcg,
ctacaaatccttgatt, ggaacgtgaggagcgt, acgcctgtaattcagg, ggggggggggaggctt,
cggtagttataaacca, tgttagccacaattgt, ccggtactttgcgggg, cccctgtcgacacccc,
ctcttagcctccaaca, ctgaccctccgggagg, aaaccactattaaaac, ggtcggggggcgcata,
ataatatccagtgatc, gaaacgttaaatgtta, ctagctgcaactgatt, tacatgtctaaatttg,
atgaggtgtctgagat, ccactatcccccccat, gggctaaggggaagtt, tcgctttaactgggaa,
tcatttcagataaggt, ggatgcacacagtgta, gcccctgccaacaaaa, aacagtaccacagcag,
aggaaagctggcggcc, taccatctgaacaggc, cacccgccccccgtca, ggagaacggcgtgacc,
tactaaggcagcttag, cttagtacctcccacc, gagatttatggtgggg, ccctcacctcatggac,
tgatgcctgcctaaaa, ccgagcattggaatat, tctctcgcacagcaat, tgagtctctcctagcc,
agggagatccagcctt, ctccttcgaatgtcca, agtggacccccccata, gggatcagegccccgt,
ttggctgtcccttacc, ctggattggatgttcc, taggcagaggaccatg, gcctttagacctccat,
tgatccccggcccctc, gttacctcattactag, tccccctcgcccctat, tttatgtccgcattcc,
tcctggagcataagac, gcgccagtgtgtgtgt, ttatgattcatccctc, cttactaagtgggtta,
atgggtcatcacagat, ttcaagacacccaacg, gtacagcaaaaaaaag, gtcggctattggccga,
cctgaagtgtgcgtct, tcttccagtagatcga, atagaactttagtgta, actgcactgctcagga,
gtaatgggggggtcag, tggaacgtgaggagcg, gggcgaagctcagggc, ccctctagttattgtc,
acctactccccccccg, tgggttccccagacgg, aaacgggggggttgtt, ccggtgttcgggtccc,
taatgcgatttgcctg, gtctattaaaacacta, tgctattctgtgagca, attacaccattaccta,
cgtctccactgagaca, cgtcaaactaaaggct, tgtctcaacaattatt, ccctgctttactggga,
ggcctccgtgcacacg, accctccgcacccgcc, atgtgagtactctttt, gtctcaggtatgggat,
gtctacttttcctata, tggatggccaggttag, aacttataacaagact, catacaagagttatgt,
tcatctaccctccagg, ttttatcgtgacgtct, tacaaggccgggcttg, aagatagcgtagctcg,
cgggcttaaaaacggt, gggggggcaaaacagg, ctctcgtgcggagccg, ggttttacataggtta,
gcgatgctgagatgct, cttgacatcctaggtg, gaatcctaacttaaat, tctaaacactagagga,
actattgcgattgaag, aattagggacccagaa, cttcccttacaccaag, gccgcctgcccctagg,
gaggtgatgccctaag, cctgggtgcccaccta, tttgggatttgcacct, tatcgtttcatcgcag,
attcctcgccgccccc, gctggatagacagact, actgagagggatcagg, taacgcttctcaattt,
gaaaatggggggtatc, aactcgtcaagtggct, acttgtacttttacca, tatacattgctcatct,
gatggccggagtaggg, gcctatgaggtaaagc, gtcccgacaggggggg, gggacgggtgcgagtg,
gggcatacaaatcttc, tgtaagtgcttcccag, gcacactagtacactc, gtcagcgactcaacac,
gtttcgtcttatcgaa, tttttatatgaaccgt, aggagagccttgatta, aatggacacgtgtact,
tggtagcaatgacttt, aaaaatggcgttgaaa, aatatgtcccattctt, ggtggggagctcctgt,
caggcaaatacttttg, catatgatgacgggca, gctctcgccccctgga, aatagcgctagcgcta,
cccagtcaaggtgcca, gtacctcggggggccg, gggttgagggcgtaga, cattgtggcacgagcc,
atattcgatgttggtc, taacgaaaaaaaacaa, gcactgaccatcatgc, acacgggcgatgctgg,
gcccaggggatacgtg, atgtttgaaggcatct, acatactctaccaccc, ccagcccacccttccg,
tggtggggagtactta, acgtgcctgggaaaca, ttttaggggggggaga, tgcctggctcagcgta,
cacaaataaaacgtga, tgcgttctcacaggat, ccagggttatgactag, gagagtggggggggca,
aaaaaagagtgcgcat, tttacagcagctcaca, aaactaagtctactaa, cataaagtagtgggtc,
tcaagcatagtggttg, caacaggggaatccga, gcgctcccccccatat, cccagtaccatcagca,
taataatttcgtatta, ctcctggggcgcccga, tcctgttaaaaggcta, gtggggaccttggcgg,
gttttaaccatctgat, ccgaaaagggaatgag, cccattcctatggacc, gggttcgtcttctcac,
gaagaataccactctc, catctaggacgtgagg, caatcccccctttcta, gcccagggggcttagg,
cttcacgatctcttta, cgtttatctaagaaat, gtctagacattgaggg, cacgtgtgttcaggcg,
ccgaaggcaagatgca, gacttagtaccttaag, ccatgcctcgggttgt, cttccgcatgcatccc,
gtcccccccccatgaa, ttcagttggaacacat, cttcctgcacgggcag, gacgtgttgaggtcca,
gcctacatacatgtaa, cttggtggcgggcagc, accagactggatcctg, gactattcaataattt,
agttccacgtgaaaga, atttatcaatcactgg, ccgtgttgtacacagt, ttttagggggggttaa,
cacgaatgggaaaatg, cacccgcatccatgga, agtacattgaacaaga, ggggggcacattgtaa,
tattaatgccccccat, tgcataaagtagtggg, catggagggtgcaact, aacctcctggttggcg,
cgagtccatcggggat, atcccttttttgtcaa, ttgtagttaaacaatt, gccggggccgggattg,
ttttcagagtcacgaa, gatgtaggatatcagt, acactatggctgggac, agggaccccctaaaca,
ctctccaacatccgta, tccactttccggttct, agtgtcgccgctcagc, ttttctaacaacaggc,
gctcccccccatattt, ccctgcggagggggct, gccgctcagcattcca, ctatttcccccccctc,
gtaccactgacggtca, acctaagaacccccaa, gactaaaaaaagtgcc, tcaagccaggtacacg,
ggtccctattttttta, ggggggggtaggcccc, gaccttcaaatttatc, ggattgagccggctga,
ccccgaaaaagtaatt, acacaatagtcaacaa, cgatcttggctaacaa, ttaatcgaagtagaca,
aatcttcagtccacaa, accttgtgccccgtct, ggagctccgcgcgggg, acttgcccatgtggag,
gatgagataggcagtc, atgaactgacctaaac, atgtgaggccctgtca, tggacaagaagaggcg,
ggcataagtttatatg, ggcccggtgggcgaag, acagggggatgctatt, tggctacatggggtaa,
cgccacttttttaaaa, actccgtagagggttt, tttccgatgacaattt, ttccgtacaatgtacc,
agaggtaagcataggc, cccatagtgaccccgc, gtatagtcatctttta, ttgctaccagccttag,
gttgaacatattgtca, gccgctcttcgaggtg, tcaaagcttccgcact, ctcttcctttgtgcgc,
tgcgcgtggcgcttac, tttgttagtacatcag, tcgctgctctgaatct, gtacttaataccatag,
aagggaattatcagtt, gggagacataagtgtc, gagtatatggtgtgtt, actgacacctcttagg,
acaagagcggtgagtc, gctatggggggattca, taatttttttcggaaa, tccacctatggtgctc,
gtgttgaggtccagac, gtagtttgttatggtg, gtggacggaagggcac, tgttagtgacgttttc,
atgatgtcttgtagct, gaggggggagcggtta, gaggctcacctcttcc, ccccccgaaaaaatca,
ttgattttgtatcgtt, gcactgccatcccgag, aggtggtagcagcacc, cccactaataggatct,
catgcgtgcagtctct, tggaccacgtctccgt, tctccaaaaggatttc, ttcatagtaagggcat,
gtatgttccaagggct, aaaacttaggagcctc, aagttagtagatatta, tgtcaccgtttttttc,
caatagggggaggctc, tctgatctaaacgcgc, ctcttggttgggtgcg, acagaaattgcccccc,
cgtggtgacgctcgcc, tgtcttactacttacg, gtgtcttggtgctaac, attgttggtggtcaaa,
tacgaggtaaatgact, cttgcgtctttatagc, gaaattaaccagtggc, ggtatctcctaggaaa,
gcagagaaggctccga, tagccttactctgggt, gtgtgccgagcctctg, ccgtcccccctctttg,
ttccaaaaaaagtggt, atattggtcgtatata, ggggacgtcgaaggca, aactgtgcgaaatgaa,
caataagttggtctaa, ccttcctgagcccgtc, taagggggggagaggc, attgcctctagctttt,
tatgaatggtgcactg, agtacatattttttac, ataacccacaacagtc, catccgatttgtcaat,
tttttgatctggactt, cttggaaatggcaatt, attagttgcaacttgc, agccgcggtccccgtc,
cgcgggacttccagcc, tccatgcctcgggttg, ctgtgtagttaacatc, cgttttttttgttaat,
gttgcggcgaggggca, ccagcaagttatcagt, cgtgattttggcttca, ggacccacatgacggg,
acaggcatggccaatg, agcttttctcaagcgt, cccgaggtcccagagt, gataggcttaaagcca,
ccccttgcctgtacaa, aatctttaaccatatc, tggccctggtaggttc, ctgttaaacaggccta,
agtcagttttttttgg, aagactcaagtaatga, cagcttaagcaggaag, tgatgtgtccattcga,
aaaataataaacgcag, atcctacgaccttgga, ccggctgggactccat, aaagttgaacgttgca,
catatatccggcatgt, aaggcggaggttcctg, tgacctaaacgcttca, atactcatcatgtcag,
ccatcggcaatgcctc, tgcccaggggatacgt, ggtcatcatcctgaag, agaatggcgttatcct,
tacccggggttcgtct, ccaatacaacatctag, tttaggggggtagaca, tgtccgtgctgaacct,
tgcccggtctactatt, cccaatcgccaccagt, cttcgtgtctgaattc, tccccgaatgctgctg,
ttttttttcggccaca, atggggggtatcagta, ctcgcgccagcagccg, gtggggctaggctgga,
aaacaaaacgcagacc, tcccactccaggatta, tatcatttatcccacc, cagagataccctaggt,
gtggtggggggataag, atttcattggagggat, gcgactcaacacatat, agtatctaattattgt,
gagcccacaaataacg, cccagcggcaggtctt, tccatctcggtttcgg, cacagtaaaaaaaagc,
cctgctcgtaaaagtc, ctagaccatcctggtg, atcttagaacgggcag, aatttgcttgaaaggg,
agacctgtgccgcgtc, tgtgataccatctgat, acgaaaaaacatgccg, catggaggtcttagca,
aaatttttggggatgc, ctatgtgattgtaagc, ccgaatttttttgagt, ataacagtgttcaata,
ttgcaggaagagcgag, gcggtccccgtctgtg, ctggagttgcgatggt, aaacaaagtcgcatag,
atactactgggtgcac, ttctttcgatccacta, cttcgcccttgtcctg, cgcattgcgccggcgc,
gagtatgggggggtgt, agttcctggtgaatca, ctataccaaaacacat, ggtttccagacatcct,
gacaaaaaaaatgccg, gcatatatccggcatg, cacctgcttactgaga, attttaaaacgcataa,
gaattttttagggggg, tccccatcatccgata, ttttgcagcggttgta, gggatgaactggtgta,
atgccagtccaaggcg, aaatggcggtgaagtt, cgcccgctcagcgcca, tatacccccccccgaa,
tcatccccatagacaa, accacagcttatataa, ccccatcatccgatat, cattttacgcctttcc,
acgcccaaaactttta, tcaatgcagttttctg, accgccccggtetccc, cctgggcgccagagtt,
tacgtgactttttgaa, atcgctttttaatgtc, ggtaagatcgctatta, ccacataaggggagaa,
gacaagataagagccc, gctgggcgctgggcgc, ctgtgtgttaagatcg, tcagaggcacgtccca,
atgacagctcactatt, agataacatactccac, tgagcagggtgattta, tggcatcaaactaaac,
tggcatgtgcccttaa, tgccacaaatttgggg, gcataattttgtaagc, gtggttaggtatttct,
agagcaaaccccttat, tcttaaaaaaaacgct, gggtagcctcctcatg, gcttccacacagacgg,
aacaggggaatccgat, agcttagattgcaaga, cctttatgtgctgcac, cgcgccccccccactc,
agcgtttcctacagga, ttattatgcaacactt, tgtgtccggaacacag, atctatgaggtccaca,
ttcaatcagtgtgtgg, aggggtattgcttcgc, gaaccccttaggtcaa, aatgatgggctgtttc,
tgtcttgctacgttga, tctccgtggctgcgca, tatgtaattttctaac, ctccacccgcggtgcc,
attgcttcattggggt, gagggggggcaaggta, tcaggttagccactag, aatcatccagacttag,
tctgacccaatctcag, tggggggggggccagt, acgtaaaatagatatg, ctatgaacagttggga,
gaagacggatgcgggt, attacttagaaggcag, tgaacatggggggggc, gtatgactggtatcca,
gtagagaatactgaaa, gtgcggaaaaaaaatc, cttagagtccctctca, cggataatttgccact,
gcagcctgtaactact, gtcgctggatctccta, caccctcctgcgggtt, ttagctaagttgcaag,
tgctcgtgatagtgcc, ttgccgcgcgccggga, tctttaatccatgagg, gccagatattggcacc,
taccgtttattatagc, taggatgttcttattg, gcgtggcggccatgaa, ccgaaaaaactaaaac,
gggaaaccccccttcc, actgtccgtttcactg, cgacagtaaagctcaa, acttgtgagttgaagc,
ttggggggggtaataa, tcacttgatcatggac, gactgcttttctagag, tggagcagaggttata,
ctccacggtactgctg, caataaaaaaacgggg, cttgagcattctgtaa, tacaaaaaacgttttg,
tcctctcaggcacggt, gcggggactcggcgcc, tctctagcagattaag, gataaaaaatgaacgc,
ggcctctggttcatgc, tcatagctaagtgcct, tcaaacgaagtggata, acacttatggtttaga,
tgcccggcgctgaggg, cggacgtttggggctc, gatttatcttacgggc, gaccccgagaactatg,
cttcaaaaaaaagtgg, aacagctcgggggcgg, ttgatgcatattagtg, aattagcactcagtgt,
actcggggtgtctaga, ggtttagtagttgaaa, tgagccaagctagcac, gtagtagtcacataca,
gctaccacagtgttga, tccccgtgggtccttg, cattaattatggagtc, tatgggggggttagag,
tcgatgttttttttgc, ctagtaatctcacagg, caacgtcacatgacag, ttacaattatggatgt,
ggtcagtactaggata, cagaaattgcccccct, cggataaggaagttct, tcatatctctcaggct,
tgactgatcataattg, ccgcaggcatgccggg, gatgcagtaaacagga, aagtcaaacgaaagaa,
tttttcgtttggtcac, atggagcaacactact, tcctgcttgcgagttt, atccttaccatcacga,
tcccttcctgagcccg, ttccgtactgatgctt, acggccgggctctgtg, acagaggagttccaac,
ctgatgtcgacagcct, ctcccaacgtcacatg, tcaagtttggtaagta, ccaaggcataaatcac,
cctaccacatacgtgg, caaccacaccctttac, caccgtggtggctgcc, tgccaaatacttatgc,
tttggcagttattggg, aaataacggttgcagt, agcgccatcgcgtggt, gtagcacacacctggt,
cgacacagccggcgct, ttcgtattaacaaaag, tatggtgacggggagt, tgccttttgcacctac,
acccggccaccctttt, aagtttgaagggctga, tatatctaaagtatcc, aggttgcccaagtcca,
taattttgcccccccc, gtccatcggggatcct, ctcgttgaatcctaac, atggccctctccactc,
acggcccgcgctaggc, cagtcttacggttttt, agtccacatgaggact, ggatggctatgaattg,
ctcccccaagggcatc, gttagctaatgcaggt, caaacatcttaagaag, atgtgccactttatgt,
gactttcggtatgtat, ataattagagccacta, aaggtgctgactcacc, atgcatgcggttgata,
tcggagctccgcgcgg, ctggttaaaccctgtc, ttaactagcctcactt, atccgatttgtcaatc,
taagggagacatacct, aggatggttagtttag, ccgcacccgtccaaaa, tacatactgacataat,
cgggtgagacagtgct, ggactgacacctctta, ttgtgtcccccctagt, atcctagtcactgcca,
cggcgcggccttgcga, cattttttagaggcta, agctttatgcctgacc, caccaaacgacaagtc,
tcccgatgtcaagaga, ctccaaatgccggagt, gacctcttcagaccac, attaagaatgtcgcaa,
aatgtgggatgacggc, gccaacacacttggca, aatgaggtgggcgttc, cgcacaacttataaca,
gtccataacttaccta, ccattaagtttaagtt, ccctgggatggtcctt, ccgctaaaaaaagtgg,
aatgttcgtatgtgga, cggaggcggccttgcc, aaacggtttatgaggt, acctggatgctgaagt,
ggataattaaacattg, gggttaaactttttta, tattgccctacctcag, ctgctgctccgagtga,
gcatacttggcaaagc, ccgtagtccgggtgca, gtatcccgggggatct, ggttaaggtctttgtc,
agaaaggggggcatag, tctttgccatcactcg, cactccagctatgacc, aagatcccggcatcct,
ggatctcagaaatgtc, gcatgcggttgataaa, ggcatcaagctccgat, aagagctgacttgcta,
tttcggggggaggttt, tgttcgggtcccggtg, atgtggctcttagctg, agctcgtgcttaatgt,
ccagtggttgttggaa, ttagactctggcactg, gtaacaaaagattaag, ctatgtactcacgacc,
cataggccagggtttt, ctgcttaacgcatgca, tcggtggaaagtcaac, aggtcctttttagtca,
gctcttgcacggtgcg, ggacaaaaaaaacgga, aaaagagaacgattct, aggaggagcgttaacg,
ggggggccgtgaagg , taggtgttccttcttc, ctcctataatcccgca, tcaattaggcctccag,
gtactcaggttatcta, ctggaatttcctcgtc, ctgtgccgctgcaacc, atagactacatctttt,
tgggaacatctcgaag, ctgtctgcggggcttc, tctattactttataag, cattgttgtcgctggt,
ccgggaaggcttaggc, ctccagttgttagttg, tgcagtccggatggtg, caattagactgccaag,
gaatgttaggtggtat, ccaaaacatcattagc, acccccccccggaagc, ttaggggggggagagt,
atacccggcaaaacta, ctctgtaagatatggc, agcttttttttgctat, catgcaccagtttccc,
aggcagtgatatacac, gattctggaagtgaca, ttccctccagcatatt, ttcggtatttaataag,
aaaccagcaggtccta, gccaataaccctgatt, ctaagtatcactacaa, ctaacatgtcacaagg,
acatctccatctgccg, tccatgttaactgtgc, caagtccggcaggcgc, gcttttggatatcttg,
gcgtggctgctggcca, tttatgccccgcccct, ataccacaagttgctt, cctgggactacaagac,
tttgtgtgcgcacatg, atgaggtacgggcgga, gggtagcagaagaacc, tgaccaacgtgtagaa,
acaaccacgacaaaaa, cttaaacaggaggaga, gcaaggtgtaagttca, gctccaagaccgactc,
gcaaaaaaacggtaat, gctaggccagtttacc, ggaggaccagtggtag, tgaattgcacccctga,
gtattgatgtcttgac, gctctaagtgcactcg, agcttcgacacggctt, ctcaaaccatgttgaa,
gaagagggggtatttg, ggattaaatcaacaat, cgggtggacaattagc, ggtttttcattagaac,
gattttgagcactggt, cagagagtactttgga, taccgattgtctttat, gttgggtgcatctttg,
cctaaaaatcagtgca, gcttacccaggagggt, gtagaccacatgtgat, cggagctcctcggctc,
gattatgtttggacta, aaggcaaccttctcgt, aagccagcctgcacga, ttactcgcctcaacct,
acaccttggtgcagga, ttgttatggtgacggg, agtctcaaagacctta, atgattcatcctttgt,
ccagtgcagtaggtta, acatatcacttccaga, ctggaagctcactagc, cgtttcccccccccaa,
gccccctccatacccc, tcgtgatagtgcctct, ggaaaccttttttgta, atctcattagtagcgt,
tctgtatcagctttcc, tgtaagacagcgtggc, taggccccccttcagt, agaggcgtattttact,
ttcactgtcagcgtag, tgcactgagagggatc, gtggtgctaccactca, agcagcacaagtatgc,
ctcttagcctcctgga, ccttaccatcacgatt, cgttatccttgttgac, atcggcaatgcctcga,
ggggttggacttttag, gcaacgcaaagcaagc, ccaccaaacgacaagt, accacgtctccgtggc,
gccacaagtatgtcca, gaagtcctgactgctt, ctcgagattcctgttt, agccttctaccatcta,
aggcacaacccaatag, gacgccggctaggccg, atggggggggctgcca, cccggataatagttta,
gttatccaataggtga, cagtgaagtttcccat, aatgggggggggtaca, tcgtcaagtggcttca,
gaactggacatccgcc, agattcgagggaagtg, tacttacctgattaaa, gcaggaaaatgtacac,
ttcattggcaagctaa, ccttcaccgcgcccgg, cctccgacagattatg, agctgttcaaggtttg,
ccacgacagactctgg, tcctccctaagggccc, aactcttacactttgt, acctaaccaaaaatac,
gcccttccaggagcgc, ttaaaaaaaggcggag, agcaaaaaaaaggcct, tgtcagtacatattgc,
ctttacttcaggccca, ggcggcggaccttcct, gttacctaacatatag, acactggtatgtacct,
aggtaaaatttaacga, tgtcccccccccagac, ggtcgggaggggttca, agtggtcctccttagt,
tgttcagcaccttaac, taagccccccccaact, atctgattcagtgaca, tttaggggggggctgg,
gtcctttttttacgtt, tctttaggtcaggaga, tgagaaagtgtgatgc, ctgttggggggggggc,
agattaagatacaatt, cgaagaaggtggatta, atggttaaggtagtcc, gcttagagcggttcct,
ctacaatccgaaatag, taagcatattggtcgt, aagtaatacgtaaaga, aagaaactagccaccc,
atatgcgtaccatttt, tatcaaaagggtgacc, tccgactgttggacgg, gagctgctgtccaaca,
ctgcctaatatggatt, atatggacttggtctt, agctctacattaaggt, tacaacccactctatc,
tttgtgcgttttttta, agagttatcacctgac, ctcaattttggcatgg, acgcttaacaacatat,
cacacccaggatgccg, taatccaagattacgc, tccccgcttagcgcag, cgtctgtgttctttca,
cggctccagaccgtgc, tcttggattaccagac, ggtaatgtacccacct, gcgcggggctgtcccg,
tcgccacagctaaggt, gacctgttgagcccag, ttctagttcaatgtag, taaagtgccaatcagg,
ccaatgtgcactccat, ggtgatggagaatgta, aagtctaattactagt, tgcgtggccatatttg,
tcgtgttatttgcaag, gagagtgctgcatatg, accaccctggctttgc, acgaaaaaaaaggccg,
gcacctagagccacct, ctccatgaacagatct, cacacataagctcggt, gttctccgccctcatt,
atgggcaacgcaaagc, tggcctcagtttcgtc, agaatctgatcacatg, ctaagttaaactgggg,
ctattgctactttctt, taccaccaaagcccct, agcaagcctctgcatc, ggggggtctccgactg,
tctgatcccagtcgtg, cagattgcacctctga, acagtctcgcaccacc, ttttagggggggcagg,
acgaaaaattggcagt, aggaacagcagcggtg, ggtggccgtgctttcc, acccaacattaagtct,
ccaataagtcttactt, aatgccttcttcaggg, atacccagtttttagc, acgatggtcggctaat,
attggggaaaaaaacg, taagcaacttatagct, ctggggcccatttagg, ctgtttcggtgagtta,
tttagatgactacact, cacctcagcgacacga, ccaagactcagttgat, cactttcttccgtaca,
ggtgatgcaatatctg, tgaggcgaggtgattg, ctgcgttgcgcacttc, aacaacctgtaatggc,
tccaagaccgactcat, gtctatctctgttaat, aaccgtgcagtgtaca, cctaatgctccccccc,
ggagcagggcttacag, attccctgaataggga, ttcattaaactgtttg, tatttataatgggggc,
cgtctagggacaaaaa, tacaggtccctatatt, gaaatgatcctacctt, atcacaggcacctccc,
tgaaaaacgatttaac, actcacacctccagtg, ccgcagcgtgaccagg, ctgggttccccagacg,
cccagccagtccaatg, agaaggagcccttagg, ttatttaaatacgtga, aatttacaaaaaacgc,
gtgtgagggggggttg, ttggcatgctagtctg, tgggcccctcgacgcc, gtttgtacccttctgt,
aggaaagaggcgtatt, ttcgtgcacagtctaa, ctgagtgagctccacc, gctaggggacccaaaa,
attcgtgcacagtcta, gcgtgccagagggcgg, attcattttttgtcga, agctaatgtctcatat,
tggacgtgttgaggtc, gacgtgatcccaatat, attgactgatcataat, gatagaacacgaaggt,
acactggggggggtgt, gtctttatctttgttc, tttatgtggagaggtg, taaggtggcttgtgaa,
cacttattgcccatgg, ttgacatccagtggcc, tgttttgccaccgtat, gccccacagacactac,
ggtctgttggtgtcag, gacggatgcgggttct, atagtcgaccctcagt, acttacggcttagtaa,
cttgggggggggagat, cgaaaaaaaatggttc, acgttttttttgtata, tttagatctaagtgac,
tgtctaactttcgagt, acggggggggaattta, agtagattttgggcat, ttccccccatcaactc,
ccgcattcatgctctg, ttgtcctgtcatcagc, agtttattcttagcca, ggtccgatctgccctc,
ttgaaacgaaaaaaat, ctccatttatttttcg, ggcaccctccacgacg, atatgtctcatcttgc,
cacggaggcacgggcg, gccccgtcttagaggt, acagtttgcgatattg, gatgggttcctaacct,
ctacccggagtgggaa, agaattgtatttcggc, tcagctatatttcgtc, acaggcctctgtgacc,
gcagtatcaactgcac, gcgattgaagcagaca, ataacacgtgagaatg, atcaatactccagtgt,
gggaaaaagtgatagg, cgtctcagaagcccat, ctatcagaaaggggac, gtgattactaacacag,
tcaattgccatttgca, ttcaatctgtcctgga, taacctgagatgtaca, tctaaactccttgcat,
taagctcagggtttat, tcatatggatgtagcc, atgataatcttgctgc, tttttgctcgcccgcc,
ccagcgttcggcctgt, cctaaggtttgagacc, tcttcagttttattcg, cccggttccctctagc,
ataaggggtggtgcaa, tcactatgtacaacta, atctaagttcctatca, caaaaaggatgagttg,
tccattctgttactcc, ttcgtgatttccttcg, ccaagcaaacttgtaa, acacgaagaagacgag,
ccataattggtctcat, ccgcacctgccgtgcc, gactcattgtaatctc, agcagagctcgtctcc,
gcacccccaggctaaa, taggtgacccccccac, caataggctgtggctg, cttggacattctgtca,
cctacaggatctcgaa, tgtcatcttatctaag, ctttttcccaaggtat, ctcataaacacccagt,
gtctgtaccaaaactt, cgcaacaaaaaaaacc, ggttacaccctcttaa, aatgtaatggataaca,
atttcaaatgtggacc, atttcgtggcctccct, tgaaaggcattccttg, agcgtgaggcggccat,
atttaacgacttcttt, actgttatcccaggag, tgggttgtagtcacca, ggtggtgggcgctctt,
ataagcatattggtcg, ttcactatacttcaac, gtaaagctacccccca, tcactgactagatttg,
aggagtcccccccccc, caggaggcccaacaca, cccccgggcgcactat, gctccaagcaactcct,
gtcccatcatttaaat, tcaaacaagagcggtg, catctcggtttcggct, cgtgaagcctgcccgg,
tctgtttgaccacctt, tagcctgtgcctccag, ctcccccacgacaaag, ccgtactgatgcttgc,
gtataccattaatctg, actgttgaacaacccc, aatcgcttaaaaatga, acatggcattcggcca,
agactgtgacgtgtgc, taccagaggatgaata, gcctcgctgatccaaa, gacccgagcactttgc,
accatatacttgaagg, ttaccttatcaataag, actgattatgtgtgac, gctggatccctgggtc,
agttggttggattaga, gcatgacgcacccact, gatgctgggaccctgt, cgggaggggttcagaa,
gtaagtctataaggac, ctgtgtaccgtctgcc, gacaaaaaaaaaacga, gagccaggggccggac,
ggttggggggggatgc, gatcttggtatgttaa, ggatttgcagtgaaca, ggttttgacctgaggt,
tattcatgtgttgtgc, gaacgtccgtcagcgt, gtacacgccccttgat, gccttagccacccgtg,
agttgggatgcctgtc, tttgaggctttaatgt, cggtgcttgccatcac, cgcccatatggcctga,
gtctgacgctgctaat, ttttaattgggcgggg, gtgggaaagggggggg, aagcttatagaggttg,
tcattgaaaaatcgta, cggaacagttatctga, tgattgtatgtctata, gccatgttttttgtag,
tgtattacccacgatg, cacactctggggcaaa, tgggcaacgcaaagca, tacaatggctcgacct,
tactctgggaggctta, caatacctatccaccc, aacagcgaccagacca, ctatttgcttagaggt,
acatgggtggcacgga, cccccacttttttatc, ggaatgactggactcg, tgccccccccggccac,
tgtctggctgtcatat, taaccttcacgatctc, gggttgtggaatcaac, tgtctctcgcacagca,
attggcagggcgcgtt, ttatccaagcatcagc, gcaggaagagcgaggg, agggggggggaagcat,
tcagcatctcatataa, tttgggaaaaacgaga, acagctaaggtcacgg, accaacacagccacta,
gtgtgaaggaaatacc, attacccaagtggggg, gggacttaagtgccgc, ccgcgttgcggcgagg,
tcatcttaggtgttcc, ggattagcaaactata, gcaggtgccggaaaat, gcaaaaaaagtggagt,
acgctaaaaaaaatgg, ttcgacatttttttat, ggtattaaacttattg, tcctaggatattatgc,
agacgctccgccttaa, ttgatctggacttagg, gggtcggggggcgcat, ggagcattgactgtga,
cctatgactcactgca, ggacatttttttgctc, tgacccacattgccta, agcccgatgcaagtcc,
cacgatagggccactt, caaggggggatatctg, tagcttttttttagtc, gacgggtgcgagtggt,
atagcagatgacacga, cgcagctgtcggtttc, tagattcgtggttgga, aaatgcgtggccatat,
ttacctttaggctcat, tgcccccccctccctt, taaattttttcacgag, acggccggggtagtgg,
tgaagacggatgcggg, agcacaatgtgcgcta, atgccatccacttcca, ggcagtttcttaatgt,
tcggaaacaaattaaa, gccaaaaaaagctgtt, aagattggggggggaa, ggtgtctgagatgtaa,
atttttccagtacccg, tcttaagttgcttagg, tgggactccatgcagt, agcctcccacggccgg,
ctcccccteggacctg, ttactgtgcagacgct, gctgagcgccatcgcg, gtatttggtacagttg,
gctcccctgctagttg, cccactattgtgatag, aacaactgagtgggtt, ggttaaaccccatatc,
agggggggagcagaag, ccgttggctccgtcct, ggtgcgcgggacttcc, ccgtcgccccgtccag,
ttaacagctcggttaa, ataagtcttacttcca, ccctcccccacgacaa, ttatgctagcatgaag,
tactccttgttgtaga, aaaatccccctagcaa, gctaggctccgcgaac, cgggggcttacccccc,
gagtctacaaagtgac, aatcgccaccagtatg, ctaaattggtaaacct, aacttaagaagcttac,
ccaggtgtacggtaca, cccggcacttcccgtc, agctccccaggttagt, ggggtacatgcactga,
tagcgatgctgtttca, gggttaagccagctgg, gtcaatgccaccgccc, ggcttaggcgggtggt,
tgtaaatatgcatgcg, aagcctccttgtttac, ttagtcatacaggaac, gacagtggtccactac,
cagagtaggcgggggc, accctacaggcttcca, attgggccccaaccat, gctcctttaaggtagg,
tcagggttcacttctt, accctcatagactact, taaggcaattactggc, ggaaaattagggggat,
ctccgcactgacactg, ataagcttctgtcaaa, ggaggcttaggccggt, cgagcctaagctggac,
cggaggcacaagaaac, gctcatttcgtcaaca, cacgtttacaatgcgg, ataaccccactgtacg,
gcatgtaccacagggg, aataccaagcgaggcc, gatcacagtaccctgt, agggttggaaatggtt,
cctgcttcgctgctgt, aaatagtgcttaattg, cctctatagtccacga, gcctccccccctgcct,
gcatcaacagctaggc, cactcgggtgtctccc, tacccctgacccctaa, gtttaggtagttggtt,
attctctcccccccct, atacgtgaccgtttgg, aaggtgctaagtgggg, agcgtcggccccgccc,
aagcttagctttaagc, gatttcccatattgct, cttagtggcgagcgcc, accgtttgagaccttt,
gctcttgtctgacaga, actccacggggggtgc, tgtgcctgtccaagtt, atctttacagttgtgg,
aaaatttcgagtctcc, aaaggacattagccta, aacgggggaaaaggat, agcaggccattctaga,
acttttctatgaggaa, tactcgggatgctgcg, tggaaaattaatcgac, gcatgcttcctcaacc,
gctatgccacatgcgt, gagattgccccccccc, ggcccctctaacctag, tcccctagttcacaga,
gcaacttcaattacac, ttcaaaaaaaaaacgg, tacactagagttcctt, tggtggttgtttcgtt,
gttcaaacatacccca, cttactaaacaatatc, cagccctcgatttggc, ttattaatcgattaaa,
agactaatgaacgtga, acgaaaaaaaaccatg, atcccagcaacttagt, gagccctttttttacc,
ctcttcgtcgggacgt, cagttttaccataaag, tattcaatgagagtca, agagcgagcaactgta,
ccctacaaggcagagg, acccacggccactact, cgaggaggttacattt, cccttcatcttcgtaa,
gatacaagttggctgt, gacatatgctaatttt, cacagccggcgctgag, tcattcaggcaaaacg,
ttgtcaccccccaagt, tgttgagttgtgtgtt, ggagggagggcataga, aggatttatcttacgg,
aggtgataacagatta, gcaagaatccatcatc, tcccattacatgctct, ccgtcacacaaagaaa,
ggggggccctgtaatg, gggcgcccccccccag, tcaccatgtaaaactc, gctagctaaccttagc,
gttcaccctcctgcgg, tttccccaactggcgc, gatgaagcagtcttta, tcaacagggaggtttt,
cccccataaagtcagt, agtaaacacttctgtg, ccagcaaaagaatttg, gcgagggttacaggtg,
ggtatgttggatgggg, gtccctcccaccagta, gagtcactaacaagag, gcaatgcaaacactcg,
cctgtcgacaccccag, ggcaatggaagtccaa, taagtgatggtaaact, cggctgttctttattt,
gataccctaggtcaac, ggaggaaaagcgtagc, tctgcctctgatgctg, gtgttaggtgtcatag,
ccatgagacaattttg, tactaaattcattgct, tcatgcctatgcccca, ctgtgtgaagcgtctt,
tgtgtcagaagtagta, gataatccacctctct, aaacagccggattgtc, tgggggcattttttta,
ttaaccagaatgtagg, atcttatcggcaaaca, gcttatattcggaatg, ctttaaacgacttcca,
gaaagaacgaaaggga, ttcttagctaatctgc, ccttatggtgctttag, tcctgtttgaagcaat,
cggcttaaagctattc, accaatattcttaatc, gggcggccttgccgcg, aggtcggggatgggcc,
tcgtcacaattattcc, gctgttctggtctgac, tatcgtgctgtatcat, ggcaaaatctacctaa,
acaaactttagattgt, accagttcatcatgcc, ttaaaattactccgca, gacacgaagaagacga,
gctatttgtttatggc, atcttcaccccgattc, ccttgcgtttgaatgt, cattgaggagcacgcc,
gcggctcggtgagtag, cctgcagttggtcggt, atacaaggccgggctt, aggatagtgctactcc,
aggtgcgtctgcccca, gcaaacgttttgtccg, gagttgcttaatgaac, tcggtttttttaaggc,
ctgaataatgccttag, aggcactggcttcttg, accaagaacaacggcc, gatggcgtctcacctt,
ggctcaggagggtaaa, ttcctggcagatacaa, gcttagcgcagctgca, actattattcaggcct,
tgatcgctagggagat, ctcaggatatccttac, atcgatgcaaaaaaat, aggtagctccattgca,
aggcccccccgagcca, ctcatgaatagacatt, tatagccaggtatgct, ttaactatgaggctgt,
aggtagcctgagaccc, gcaagggtctgcgggt, tctgcccggcccgtcg, ggaatggacaagctgg,
tctaagaacatagtct, tgagagtgtgcattag, ggtagggctgggaaat, agagttatgactatat,
gggtgtggtggatcct, ggagttaggtagactc, tggaccctctcggtga, agagtccgtgaagtgt,
gggctccaataggggg, tagagcttgcctgtaa, cttcaaagccgtccct, gttgcagtgcttgtct,
ttggaggcttagtagg, gggttttgtgcatcaa, cttagacccagaatta, gggggggggacagctg,
ccgacattttttttgc, tcactttctggtcgag, ctgaagattatcacaa, cactgccatcccgagc,
aatttccccccagcag, cttatagccccttagt, tagttatgtggttctc, ggcagggttgtaatat,
taaccccccggtgttg, ctcggcggctggacat, agtggtggcagatgcg, tagggggggggtcagc,
tagacgctggacccac, atagattaacgtagag, tttcccactgacactc, tctgctgactaaacat,
agcttttgcccattac, gcaaaatgagactatg, gagatagtcagtgctg, ctaacacatgactgtc,
cttatacccttgtcag, gcatgggttatgcagc, aagtacatacctaaga, agcttccccccccagc,
aagtactggtgtgggg, ccccatatatcaggct, aacgcatgcaaagcaa, aagtctaggtaattat,
aactcccatagcactc, ctaaatactccaatgt, ttctttttacacgtaa, tggtctaagacaagct,
gggactggatctccca, ggatgggctgtggtta, accgtggtggctgcct, gctcgcgccagcagcc,
tgaggaccgcaggtct, gccttgcgtttgaatg, gctcttttaatgataa, ggatccccgtgtatcc,
tcctgcagtatagtta, tgtctcgtggtcccag, tagtagcgatgctgtt, ctggccaaggaacctg,
ttggtctttgccaacc, aggcatgccgggggct, cactgacggtcacact, atattttaatatcgca,
aataaccgagaagata, gacgcatgcctgtaaa, tttccctaggacaaag, ttgagtcacagtgcac,
tttggagggggggtct, ttaaaccctcatgaat, gctgaagacttcaacc, ggccttaggtcaggag,
acgggggaaaaggatt, aacaggtgcatctctc, agttttgatctatata, accctcctgcgggttt,
gtagttacagttgtag, ggcttatggtctcgct, aacccagagtgcactc, cagatcaggaccactt,
gccgggggggctaaca, gacctacgaggtattt, gtggtcgcatgaatct, tgccaagatcccggca,
gtaggtaatcataagg, cggaaatcataagtcg, tttcttgcattgacta, gaggggcattcattgc,
cgtttgtacccttctg, aggcagttgatgtaaa, ttttacactcgaaaga, cggctacggatgacct,
aagagcattacttagt, cttttgggcaatcccc, cgttgagcacggggga, agggtgctggcccctg,
ggaatttcgtggcctc, acataggtagcaggta, aattgttatggccaaa, gacttgtttttaccca,
aagtgatcatctatta, tcgggaggcttagtta, gggggggatgggcact, ccttacagtcttctta,
atccggagcagcgtct, ttacgcccaaaacttt, gacttttcgcagaatt, tccatcttgaatctgt,
gcaacgctgcacagtt, tcaatcacctgaaaca, aaagaaacttaggtcc, agtttttccttgtacc,
ggtgaatttagtgttt, tgtgcattgctgaatc, ctagttgcccttaaag, cgaccgatgaaaggag,
atagccaggatggttc, cagtctttagacatgt, ttttacaggcagtctg, atgctagactccataa,
tattgccccaaatatg, tcagttagaacctata, gcttttgccggcttag, gaattgacaatttact,
gtgtcataagctggat, acctccagcttgcata, caggcatgtgaccacg, gatgagatcttgtttg,
ggctcaaaatttcagg, catgagcccccgaatg, actccttcgtctaact, cagaccccgccctcgc,
cccggcccgtcgcccc, tgggccggttccctgg, cactgcagtgggtccg, tgtcatatcctatgac,
tagtctactgtaagaa, atccccgcagctcctc, gttggggggggaccac, aaatagtttaaagcgt,
gagttacacataggct, ctcagaacgtaggaga, gagtaagagccctcgg, gtgccatcttttaagt,
tactgcccccccccat, agtatagtttgtactg, gaagatggggggatct, acgtttagctagattg,
cacaccaccttactaa, ttcagcaaagtatcgc, gaaattatttacgaga, gtagtgatacataaaa,
taccagtcaaatagag, aacaaactgggttcta, ccagtcacggtgggtc, tgccatgttctgcaag,
gtgaggcccgggggtc, tctgactgaggtatgt, aagctgtacagccata, cctgaatctgcgttat,
acccccccccttctgt, agtagagaccggggtc, tagaggggggggaag, ttagggggggggagggt,
acatcttccctattta, gatgatccattcttat, tattctgataatacga, tcttcacgtgtgttca,
gttcggcctgtgatta, gcttatgaagaagcat, gagcttggcccggaga, taacttggggggaatt,
ttaattgaggttgaag, tggttaaaccccatat, gataggaagagctaat, gacgaaaatgaggggg,
gctcagcattccatgc, ccttagtcttactcaa, gtctggcggatcactc, gacagaggggcaaata,
tccgcccaactttgcc, tcaaaaaacgaagccc, taacactgggcccgcc, attgtccaacactggg,
tcctgaggcactaccc, acagctctgctccact, cggccggggtagtggc, gtatatacatgggcgg,
ttccacatggtgtggt, ggacgtgttttctcct, tatggcctgtaacctc, atcagacctacagtag,
ccgtggaccctctcgg, atgtgtgctatacaac, aaagtaccatttgcct, actgaagatgtatcaa,
aatctagatagctaat, atctcatagtggctga, agaagtaacttaaacc, tgtaactcgtgtaacc,
tgtgaggggggtgttc, acatcttcattgtcat, ggcacttcttgtatgg, cgtgtgggaatgctca,
cgtcttttttttagac, cggaaatatatattta, cattatgaatgctgca, gtatcccccccccgaa,
acacaagggggggaga, ttattaagcttgtttg, gtggcctggaatattg, atcccgggggggcccg,
cacggtgtctaacacc, agccaggggccggact, cacctttcccgttcaa, accaaagtgttatatc,
gagataagggggggat, ggtaggaggcacagga, catcccatctaggacg, cacggccggggtagtg,
tgaaacccctggctcg, gctcggaagggaacag, cccccacaaggtgggt, gcgtgcactattatgg,
tggtatgggggggtag, agaccaggggcctagg, gagtctctagcctgtc, catacgtggtgcgtgg,
caaggttcatctaaat, aaacgaccgatgaaag, tattaaacatgttgag, aaggggcttaacacct,
tagaaaaaaaacggcc, tagaaaaaaaacggct, cccagcaaactaatca, aggagactctggtgtc,
aatgcttcccctctta, caatctgcagtctagg, gactcaattaacccaa, cggggtactctgccta,
ccggcatgtagaaatc, caagtatcccaagccc, tgatgggggggaagat, tatattcacactagat,
aacatatatactgatg, ttccagcagcggcggg, atcaactcagatataa, gcaaaaaaagtggtat,
gactttacttagggga, gaactggcgaccctaa, aggctgcgaggcttcc, ggtgattcgattttga,
gacagatagggagcag, atgtactacctttgcc, cacggacctttgcgtt, attatgtttaacgctt,
gtttgggtgtcttagg, caatgcaaacactcgt, cccggcgctgaggggt, cggggggggcgttact,
ttaaaccccgtcacta, gtgccccaaaaataca, agaatggttgtaacta, acaggggtggtagtgc,
gtgaaacttagtccat, aggtgaaagatcctga, taacaccaaagaaccc, cgtctccgtggctgcg,
actttaggcttaagaa, ctgatacccagccttt, atatgcatgttagcga, atttacgccttccttt,
gttaaacttgacaaca, aataaccctattcctt, tgggacttcgaggcag, gttgaaaataatgcga,
aagtctgtagatcttc, tttagttgtgagccca, attcaggacccacatg, gtgatcattattcctc,
gcaatatggctagacc, agggatgaacttaggt, tcagatacctttggtc, aaatcatagtaaccag,
ttatggaaggttgtgt, agggaggtaataacct, acgtctccatattttg, tggttattcagtagca,
cgcgcccgctcagcgc, tagcgacaaaaaaaca, caagcttagtccagag, ttgtgcaatactatat,
gtgtgtctatacagac, aaagataagggtgcca, atgagtagattgctaa, attaggccgggagctg,
gagctaagcggtgagg, agcgcttctctgaaac, aaggtactgcttcccc, gtgcgtatttgatgaa,
tgagctcgttgcaacc, tgagacctgtgccgcg, gcatacaaaaaaaacg, ttactcagaggattgt,
actgctgagataggag, cgttttgagctgtgtt, aagaccgactcatctt, gccagtttaccctcct,
tattgtggccctgtag, tccttcactcaaacca, cttgcaaaaaaatagc, tgcagttgggatgcct,
ggtgacttatacgaaa, ccttaaagtctggacc, tcctgtttcgagggct, tcaccaacccccccca,
gattgagaacatgcga, aaatcaatcaatgctc, attataatggcagctg, tgcgatatatatatac,
tttctggaactatggc, gggcagatagggggag, ttgggggggctttaac, acgactttgattctgg,
gattagagactgcagc, attactgataactgtg, gaggcttaggtagggg, gttgcttctatttgat,
tatgaataaccgtgtt, gaccgggcgctaggcg, aaatagaataacgttt, aatattcgtttgtact,
cgtgttgaggtccaga, gtttgaccgcgttagc, cagggggcttaggcac, ctccgttttttttagc,
ttacggataatttgcc, caaaagccccaatatc, aaatttgacctctgac, ccaggagattaatgcc,
aaattatttacgagag, ggacacccaccgatgt, tgctttaagtgttcgt, ttaatggagagcgtct,
gtgctcccgtcaacag, tctacctaacattaaa, gtattaattaattcta, tttccccgaactgaaa,
gaaccttctgtacatg, tgcactggacatttag, cggggggggcggggta, taagtcctgttagggt,
ttgattgagtcgatgt, aaatcgtgtagaacgt, ggcatgggtaggttag, gataccgagtgtcgat,
tcggataagatgctta, agggtggtagtagccc, cgggcggcggaccttc, aatccacaccaagaag,
ataggtagcttgtatc, acggacctttgcgttg, tggtaccagtaaccaa, tgttagttgcttcacc,
agactgcagttatccg, agtagcgatgctgttt, agttctctgtatctgg, agtaggatgctcaact,
tcacaaaaaaatgggt, atcgctaaaaaaatct, atgtcactgtgctcgc, acctcatgggggggag,
catgtgtgcattatga, accaaagtgcagattg, cattgaacataattca, gcacgcagctgccgtg,
gattcctcttgagagg, aagattcccacttaga, gtcttataaggttata, gcttccccccccatat,
taggcttagtggggaa, tgacgataaaaaaaat, tgggtttacgcatccc, gattctttcgatccac,
ccccccgggcgcacta, gaaatgttgtccatgc, gctctttgtgagggtt, actgttcgggttatga,
acggactgtaaatcat, cacacatagtccctcc, ctaaatcaacagaatc, cgtattaacaaaagta,
tccagaccgtgcggcc, ctccagttgcgttatt, gctggggtcgcagcct, cgtgtatcccggggga,
tcttatgaaaacgtat, ccaggctccgcctacc, gttgtgatattccagt, tgggcaacacaaagcg,
cctgaggagaccatta, aactgacgtatataga, agaacacccgggtgga, cacgggtgtgcctaag,
ctggaaatgatcaatc, acaatacgcccggcta, cccccgaatgaacccc, tccccctatctgggaa,
ttttgattagaaccct, ctcttgttttgcggtg, tcaaccgttgatcttg, gaaaccaggtgataat,
tgagacagcaagcagc, ggcttgtagtaaccag, cactgcaatgtgcagt, gattacgggtgcactc,
tcaagtggcaaggtct, acatccagcatgtata, taggtcattacttgtt, gattttgggcacatct,
tacgcccacggattct, aaataggactggactg, ctatcagtgcttctgt, agctgaggaacgcatg,
tctacatcaggtccaa, gtaaaaaaagtgtcca, acccatgttaagtcct, aacacgtgcttgagag,
agggtcttaaaagaat, atgtcacggaaaaaat, accatcgaggtctagt, ttgttaaaaacgttac,
taacagacctctacaa, gcattagaatcgtttg, cctagatggtccacgc, ctaattaccatggtag,
tcacatgctggtttat, tctgtgttataccgtc, aggcggtctgatcacc, atatctcagtgccgga,
cccccccacaggaggg, agggtgcttaggcagg, ttctgtctcgaaacac, atgctctttgggatgt,
tgagctccctgaggta, gccatttgtggcacca, ctggttgtgaagtgtt, cccgcacatctttttt,
ctcgtggcccgaggca, actagctaagagccaa, acataatgccggcagt, agtgttgggacagcta,
cccgccccccgtcaat, tgagccctttttttac, gcccccaactcttact, gaataacgggggatgg,
gggtacattcctccta, agtcagtgggtcaact, getaggtgggaggctt, tgtctccctgtaaaag,
ctaccggaacattctg, ggtccagcgcctactt, tgacacgagttagact, gacccccctctctgtc,
caactacactcactat, taccgtccttttaaaa, gacattagatcagatt, ctccagctgaatgtac,
ggtcagcgactcaaca, ggtatcttgttaagta, caggcaaccatccggt, ttagtgggccacttaa,
ctggcagtgagcgaag, gtgacggctttggcaa, gctgcgcagctgtcgg, acacactggggggggt,
tccaaaagtagccagg, taggtattgtaatctg, ctagcgctataactcc, ccgaaaatgtaacagt,
tatcctttagcagtcg, cttaggaacagctaat, cagagttagaccccca, cagagttagacccccc,
actccgtcacacaaag, agagacgtgttttcaa, caggttgcttatctga, ggctcttcccaatgaa,
caccgtgcatcgtatt, ttaatctcaatatcgc, gacacctgccacctta, ctccgcaggcatgccg,
cagctgattgggctag, tcctagacaacttagg, ttctactatctgtgat, agtgtgcgagaaggca,
tttacactatgagaac, gtagacacatcagctc, ccatcaccaaccactt, atctgggtggaactgg,
tggctgtatacggtgg, tacccaagtggggggg, tgtgaagcaacttact, acgccttgcgttcgcc,
tttcagattagcgtac, gagatcctccattaga, aaatcgtatgtcataa, gaagccacttatagat,
tggtgggagtttagca, ggctaccatcaacagg, ttaggtaccgggctca, ctaggggcaccacgat,
tggtggggggggaatg, attgttcacacttaat, agccatcttctccggc, aactgccatgtcaccg,
tatgttgttaggttct, agaattcttacgggct, aggctcatgcatggcg, atgcaggctatgccca,
tgcatgtaatcccttt, tctcccagtcagtatg, ttgtggtgactaatca, caggttcctatacttc,
gtcttgatgtacttcc, ccccactagaggcatt, tctatcttgcacaatg, gttccccgaaaaaaat,
aatcggttgtcttcag, aaaaaacatgccacga, gtgtacaaatcaaacc, actacatggtcttggt,
gagtcggtttttttta, ccacggggggtgcagt, gtaaccgccccggtct, cttccctctggaccta,
ataccgtctctggtcc, agaactgattaatgct, gcagttagggatcccc, tttagcccggttccct,
aaccactatatcagca, ccaggcactaggtatt, gcaaaaaaatagcgtg, acactcccaggtctta,
tgggcaaagggcgtgt, cggaagacccaagccc, ctacctttgggtgact, gagggctgaggccttt,
atgttgtgggcaccct, taataatgatgggctg, gatgcactccaagaca, gaaaggggggggcatt,
agtctagttttgagta, agcggagcgaggcgct, gtattgtgaccctgac, aggctaggctgcatcc,
cggggttttgatattt, ctgccagcgggcgctg, gacttaggagtgaatg, tgttaggataccaatt,
tctagaaaccgtgcag, tcatgccaatgggcag, agcttggagcatacga, cttaccatgcatggcc,
agcctagtggatgtca, atctcgtgaaccctgg, ttcgatgtttggctgt, agtcagagtgggggga,
ttggctcctggaatgg, ttacaattccccttca, atgccaaagcgccttg, cgtcttatcgaatttc,
cagttaggtggagggc, tcgggggcgaaaaatc, cctgcacagctcgctt, ttctttgttatgctcc,
ctcccaaaccaatggc, ggagtcagctgtcaat, ccagcacctcattaac, cagcaggagctttcat,
gctattaaatgatctc, ttatactatgtcttgc, gggatactcaggcatg, gtgttgaaatactatc,
ccaacaatacttctat, aataatggtctgacgc, acggaactcatttttt, gtgacacgagttagac,
cttgactctggtggtt, caatggtgtacatgcc, tttacteggttgccca, gcctcggcctttctct,
ccaacttggttgatta, caatctggttaaaccc, ggacttgaacacatgc, ggcaaaaaaaaggtgt,
agtgcttgctaagggc, gagttgggtttgtagg, gtattaccgtgtgagc, ggttccactgtgatgc,
tgttggtgacaggcac, ttgcagtacactgtaa, gcagagcccgtgcctt, ggcaaaaggcctccag,
atggaaaattaatcga, gacgcctgcgtgtacc, taagatcatggtattc, tccccctcggacctgt,
atccgcccaactttgc, gagaagtttcgggaaa, gcgtagtgactaagca, ccgaggtacctcgggg,
cagaaaaaacggaccc, gtctttgttacctaga, gcaccggttttcaggg, gggctattttttttac,
tagagccccgtttgta, gggcgacatcttaaga, ccgttatttgaaatta, tctttagtaacacctg,
ttcccagttccttaag, ttagggtcgattttaa, tgcatagtaaagaagt, cctcataagagattta,
ttacccaagtgggggg, accctggcgcttggaa, ctaaaaaaaatcgaaa, acggcttttttttccc,
ctcctgcctcggacag, ccaatatacaccgggc, ggaaaagaacaagcgt, gtggaaaagctagggg,
tgccgagtagtattca, ctctgggttgacaggt, gcggggttgtggaatc, cattatcccccgccac,
ggtgcccctctatgcc, gttccttattattgag, cacaaatgaatctagt, ccccgaaaaaaggtaa,
ttgtgttgtaggcacc, ggggggggataataac, ctagaccgggtggtgg, attttgatggacaaat,
cccatgataaacatcc, cgggggccccccgagt, gagtagcctggactaa, tggtgaccatgtggca,
tgggtaacccagtcta, actgggatagcaggta, gttctaaagataccca, tgtccatagaatggtc,
gtttattagtgctttg, gggcgaaaaatcaata, gttcaacgtaatgttt, tccgctgtacgactcc,
gtgagggactaaaagc, ataaaaaaaacgtgtg, agcacatctagagaca, cttggggtgagccgac,
ttccccccccattccc, acccccatgggtacgg, tgaggtaccctctccc, cacagatacaaatagg,
taagtttgcacctgtc, ttactccttaaaggaa, tgtccgtgtctgttcc, atataacccgagaaac,
catgttgccgtagtcc, cctgagtggggggggg, gtctcttacttgttga, gtagggggttcttctt,
cgtgagagcaggttgc, taggcctaaaaaaagc, tgtatggtccatttga, catggtgtcggcttcc,
gttgaacttggtcttt, atgttagtccattaaa, ggtgaacagattagga, tttgagccagtaagta,
ataagttggtctaaat, catgaaacaggggagc, tcctcgtagagacctt, ttggatatgagtgaag,
ccacatagctatgtat, gacatccacagccaaa, ccgctctgcctggtag, gctgcccatacacgaa,
ctttgccacctctact, cgcgggaccgggcgct, atggggggggaccaga, catgggaacgcactgg,
aggctcccgtgggtgc, aatgcaggctatgccc, agagtataattgcact, agaggcgactcatgtt,
gaaatagaggttcatt, acatggctgagggtta, catcctacgaccttgg, tggtcttcttatctta,
acctatttcgattgaa, atgctcagggcaaccg, gtaaaggagcaaaatt, ggacttatgagtgagt,
taagacatcaacctct, aggtgtatgagtttca, cgtcgtcctcgagatt, cgtcttttaagtcctt,
aacgacaaatcgtatg, ctgccactgtaacact, tcacttagtggtttat, ctatactatgaggtac,
atatacgttcttgtta, ataactgggggaaaac, gaggtatcaagccagg, acatacgtggtgcgtg,
tcaattttttagtaac, tgactttatacccaag, acatcacctaatttca, cgaaaaaaagaatgac,
gaggggccggggttaa, ttagcgctcaatccct, ttagaccctgtcacac, ctaaataatccaatct,
cctactggccccttgc, tctaggggggcagaag, cgggctaggtggcact, caaccaggtctgacaa,
tgatagatttccgaag, tgcatgaaacttggtt, tctgtttccgaacccc, aagcctttagaacccc,
aaaagtagcgcgaggc, tttttgtaccccgatg, tggtggtcgcctctaa, gttttttgttggataa,
acggtctaattaaaat, tctaacctggttatga, cctgtttagattagat, gttatgtctccattcg,
cctggccattggcagt, tatagctccttagtgt, gggagtccagccttgt, atctaggcccacctta,
tgattacctttcacta, ggagggtctagccttt, ttgtacactctgtgtg, cctcagctggtacctt,
cgaaaaaaagagacaa, ccattaaaacagtacc, tggtataagattaggg, ggaaatcccttgagta,
gtgaggggggagcggt, agaaattcagcggtgc, ggctttttgctcgccc, aatgcctagtgcatac,
cattagtccggttcac, gatcatgtcacgggac, tctgagctataatcac, ttactgtactggccaa,
aaagtccaaattctcg, ggagcattccattgtt, gtgaggggggggggcg, ttacccatcaccaatc,
ggccttgccgcgcccg, atctacccgcctggcc, ccggcgctgaggggtg, cttcttagtttaggcc,
gtaaaaaggtcagggg, gaacactgccgagtta, cgaggcctaggggcct, tctaaaaaaatgcgtc,
ggttcaaacatacccc, ttattctatatgaagc, aggacctgtccactgc, caagctggtggaggat,
aatttgcttcatgagt, cccgggacgggtgtgc, gacccttaggccttta, cccatcgggaaggcag,
ctgataaaaaaagtgc, gagtcaagatagaaca, gtggggagctcctgta, caggtcctttttagtc,
ctggattaatgggaca, tccttcgaatgtccat, accattacagttaaca, cctccccccccgctcc,
aagcgtctaggctcag, ctgcgggtagtgcagt, taagaaccccaaagtc, tgaacctctcaggctc,
ggtgggcccttcacat, tttctcatcattagtc, atttcaggaggggcaa, actccccccccgggta,
gggcttagagggagcg, gttgtataattgaact, ctcttcgaggtgctaa, tggaataacgggggat,
gtgggccccccccatg, gccccccccgcacaca, cttcatccaacggatg, aatccttaaggtatct,
acccttaggcctttaa, aagataatgtgtaaga, agtttagggagcatag, gagttagtttttaccc,
gagtgttttaccatgt, gcaggtatcccccagc, gatgatactcaaaaac, ctcaggggggcattta,
attacctatataaccc, gagaggtactggcaaa, ggacgtgttgaggtcc, cttaccatcgaggtct,
caggtgtatctgtaaa, ctgtctccttcgaatg, tttgtataagtgaggt, tgatgcacgggctctt,
gtgtcataatagaata, gttgataagattcctt, cgtggtagtctatact, tgaaatttccccgaac,
ccategttectccctc, tcacttagagctacgc, tgtgctatctgtgtcc, ctcttcctggcaagtc,
acaatattctttgtga, gagagttgtaaaggca, tccgcacctctgcctc, ctcgggcagtatctta,
ggggccgtttttttcc, ctgttggaagcttaac, taggactcagtccaga, tgacagtggtccacta,
gtattgaacagatcag, gctccctctagagtct, ccgtgcgggtcctgat, accctatcatcaatat,
ctatggcttatagcat, atgtccaccaaacgac, gttttttgtgcaaatt, ctcagtcactttataa,
ataaacgggacttaaa, tacagacgttagacac, agatccccgcagctcc, agccacttatagataa,
tgtccgcctatgattg, agggctaaaatcggaa, aggtttggatttcctg, ctcgcccggccactgg,
ggaatagttgttgatg, attaccctagcccttg, gggtgtggttatacat, ggtggtggagagccgg,
cctaaaaaaagactat, ataggtgtaaggcagt, caaggggggggtcaca, agttccggttcaggac,
gagccaggaggttagg, caaagactgtagggca, acaggcatatagagat, gagttaacaccacatt,
gctctggtgcttttaa, ctcaggcagtatgttc, cagttgggatgcctgt, aatcccatcattatgg,
tggctcaccagtgcac, gcagctccccccccaa, acttactgcaaaacgg, tttgactgctaacagg,
actggactcgagaggt, agtaggagcctccgct, gtacataggagggatt, gcaggggcctgctcaa,
gctgtttgtccaccta, acccccccccacattg, attcccccctattctt, ggctcatgcatggcgg,
gtccacgttaaatcaa, tggctagacctcgtct, tcctagctgggctaat, gtttccttaggcttac,
tttgccatccccccct, ccttgttacccgttcc, gttcttgggcatcacg, ctatttgattagctct,
cgcaggactatccaac, tccacaccctcgtcct, tttgtcgctcaaataa, gggagggggctaggcc,
gtgattgtaagccaat, ccgtacaatgtaccca, ggccggactctgccag, gctcggtggtttacgc,
cacccttgaaattttt, tcatgtcttgtgtagc, gcttaggctgagggag, ggcatgggggggaaag,
tgtaaaactccggttc, cttgtgaaccgccctc, gccggtgcacacagcc, acttgagggtcgcatc,
attggggcccacccct, cacctctcaaattgac, tttttctgacctacga, aatagaattggatcta,
accccggtagcgtgaa, taagtgtgtgaaagca, gacacagattaagcta, aggcaaggagccggga,
tgaacatgtcatctgg, gtcaaaaatgggttgt, cagaacagtaattagc, tcacctgggggggcgt,
gaaagatacccttaaa, actcagggtatgtcgt, ccttggaacgaggtag, atgtatccctctatcc,
cactgccgagttatta, tgcttccccaaccatt, ctctcacgcccacgga, cgtgcccccccccgat,
tctaaagtacttgtgc, gtctgacgtctgtaat, ttatctgtattgaagg, tccggaatggtttagt,
cgaaggaaacaaaaac, tcccatcatgccacgt, ccgtgccggatccctg, taattagattaggttc,
actgtaatgtaagtct, gttataatgataacgc, atggatatatatgctg, cgaacacttggtggaa,
tactaatatgtaccta, tgtgctcttcaaactg, agccgcctgcccctag, gtgttaccactataga,
ctctgtgggattagtg, tgtcatgcctcagtag, cttttaagccatagcc, taaggcccttggttct,
gtttaattgattacag, tatgcatgaaagcaat, acaaggtcagattagc, ttgtgggagactatgt,
gggggggttctattgc, atgaggtattgtggac, tgaacctgttttttgg, aacacccccccattcc,
ctgcaccaaacgtctg, agttatctcttctaac, tatgcaataatcagac, gattgagccggctgag,
ggtaggccaatgcagg, tgtgtctgcatcctgt, acgaaagaatgggaaa, ctttgttgtcccttat,
tattggagggggttca, agctcctatctgggtc, attatgcaaggcggaa, gtctactgactctaat,
ggagagttttttttgg, cttaaaatgacgcaat, ggacgcagagagaccc, tgccgcccattgggag,
tcaggttgtgtctcta, gagtttgtgtaatgct, ccgccccccccgaaaa, gcctttttttacccct,
ctacatacacctgcca, aacggtttatgaggtt, cccgtgtggctgccta, ggtgcaccctaatggc,
ggacctgggagatggt, gtgcgtgcgccgcggg, cacttcgcccggctta, ggggttttcagggggt,
aagtgcttactaatga, ttacgccttcctttgt, ccagcatattagatgc, cggtacctggctttcc,
aacataaagagacgaa, gtagagccccaacttt, ctgaggatctggtctg, gtcatattcatggttg,
ctgatgacatgcctag, tgggagcttttaccat, ttcgcattgacctttc, tcgccaacctgcagct,
gacgtgtgtcatatgt, agtatgatctgatctc, ttcacgattcttactt, gcactctctcatgtaa,
aaaccaatcagccacc, ctctttcctgtcgacc, ctgtccagttgagctg, ccaacagtaacacagt,
atggccccccccggtg, gtgtattacccacgat, accgaggtgggccctt, agccgagtttggctgg,
ctccatatccagtatt, aggtgccggtgtgagg, cttactgtcactataa, gttacacacgcctgta,
caaaacagctacgcca, cagagacgatgcagcg, aatataaaaaaaacgt, agaatatctattatcc,
gagtagattgctaaaa, ccggtctcccccttat, ggtgtagcttcaatct, aagattagatctgggt,
ggcattagatggatct, aggggggggggattca, agaggcagaaatcggg, ccggatctaatctgct,
gagctagagcattagg, ttcccgcttaggctgg, cacattgggctctgcg, ttgttaaaaaaaccac,
gtggggggctcaagtt, cttagacttaagttag, gccagttgagcaatgc, cctgcagtcgttcttt,
tagggctgtgcaatat, gaaacgggggagattg, ggcatgcggaatgggg, gggcgaagtcaccttg,
aaggaagcgtattgta, aagtgcctttttccgg, accctgatgcccgtca, atgcccatagaaaggg,
ctcaggtaatgtaccc, cctatattgtggggaa, ggactaggttgtggtt, agctgctagataggct,
gggaactagaaaaacc, acaatgagacacctta, gaccagcacaatccag, gattatgtttaacctc,
ctatcttttatacttc, ggtgacccgagcactt, gatatttcggtattta, caggcgcctgagaaac,
ctgtatccccccccaa, cgataattaaatataa, agggagaggatcgctg, tgcgttctcattagat,
actgagccttatagat, gacgccctgtccggga, aagctgcctgaaactt, ctactgggaggcttag,
agcctccctcatgtta, acttacagaatttctc, ggaggcctctcttagg, cgtgaaccctgaggcg,
ggtgtacacagtttgc, aatctctgcacctcca, ggaactaagtcaataa, agctatagagagcctt,
ccgggggtcccctaac, aagagtgtcatctgtt, ccctgtagtgtagtga, catcactttctgtcaa,
gactcgcgccactact, attagttctaggaagc, ttgccttagcagctgg, tcgccccttttttttg,
ttaaaaagcactatct, gaggcggccttgccgc, aaggagttgggttata, atgggggggatgtatt,
ctcttctttcctcgtt, tctccggcagttgtaa, gttttagccatgatgg, aaggcactgaatgcat,
ttaatactggtggctt, aagggcttgtatgcag, cccacgtatcagtgat, tttccagggggggcat,
tggttaaactaagggg, gatgaaaagggttgat, gtgagagttaccgtaa, ccacgtttacaatgcg,
tctttatgaggagcgt, ctataaggtgctttca, gcaactattagcagtt, cgatggagaagagatc,
cttgtgcctcgtcctc, attgtcagtaagtaat, tttggctggggcgacc, ggaccatgctgattaa,
ttacgacatactttaa, cctatgctacgccatg, cccgaaaaaaaagagg, gtttttcgtagtaact,
gcccggctgctgcaac, tgactaaaccaaggag, ggcaaacttaggcagg, gccacaggcccgattt,
gagacgacaagagatc, gaaactagggaacggg, cttgtctaggcaagac, ctgcttaggtggttca,
tggtagtcagtcttag, ttcatcccccagtgat, tcccttcagacttgca, cgggtgcgagtggtgt,
ttagcatccaagatgt, tgcacatgacaacctg, aggcaaactatgtcaa, acagatccaacactgt,
tttttttacactcgaa, ggcagttatcgacaag, tatatgaaatttctcg, actggcttccccgctt,
cgagttcaagggattg, ggctatctattcggtt, ccggaatggtgaggac, tcctgtacctacatgt,
atttccctttcctcgc, atatttagcaccaagg, ccattgtggacggaag, atgacctactcctaca,
tccgacagattatggc, gatctcgttagaccca, aagataagctgttagg, ttagctagtaatctaa,
ggcgttttttttaagt, atgacaaatacaccga, ggaaaacggtattaaa, ctgacacataggtggt,
caaaccatcacttttt, gtggttgtttcgttat, ggctttccagccactc, gctatgcatccacatt,
tggaggcctaaagtac, gtttaacgtgttagcc, gctcctggtgacccga, ttcacttcataaagtg,
agaagcaaaaccgtgt, cctgccgcccattggg, actgctcaatagaaca, cacccaagtttaggga,
agtggaagtgtagtag, acgcatctattttttt, aaacttaattaccaca, catgcctatgctcaaa,
gtatgtgtccataact, cgttaccactttccat, cttcctacttgacgaa, aacactctagatacta,
tctcactctcttcgtt, tccgttatccaccagc, ggctcgtctggagtct, gatgcttgcgagcata,
taccacgctttctatg, ccgtacacccagcttt, tatactgagaaggtcc, gccaggaagacgtgtt,
acaacggtgaaacacc, attgtgttggtgaaac, gctagggggggagcag, gatgccctgtctgacc,
aaccagtgcactggtc, cctcatgattcgactc, tagtattacgtatcta, tcagctgcaactaggg,
taacggtaaaaatgtg, cttcacaaaccttact, ttcgattgaaacccaa, gtcttgaccttaggtg,
ggaaatcgaaaccgtc, ctctttactttgtgcc, ttgggtgaagacggat, gccaaaatttaaggct,
tgagaggcagagtccg, gtatttgtaggaagta, ccatcccgagcatgga, gcctttttccggctat,
aaaggtactgcttccc, gctgcatgcataccca, tgccatactcacctat, ccgccctgggggggca,
atgtcggaccaactat, gccttgtctcagtagc, tatctggtgcttgtca, cagcaaaaacgatcca,
gacaagctacagcgtt, aacaattctatgttga, cggttcaggacaacca, agttatgggaaggttt,
ggcctaggcttgggtc, gagcttgtaagttttt, gctgaattgctggtct, actagcagttctaatc,
ccatttatcttatgta, tctccatacccggggc, tctcggggttgttgtg, atttcccctggtcctg,
ttactcgtaaggctga, gtatgttcataactgt, tagtactgttgaactt, aggcctagctgataag,
tagggaagcttaggtg, aaggattggtgttgtt, caccctggcgcttgga, caatatcttcccatca,
gcatgatgcagcatac, cacccccgatacgagt, cttaatgcaggtcaaa, caagtcagatctttta,
ataggtctctggaact, ttctctattaacgaat, atctaattgtagtcat, ggattatttaaacaga,
ctatactggagaatca, acaagctcctttcagg, gagcaacgccgaagac, gatgcttaggcaggga,
ttagctgggaggagac, gcgctagcgctataac, ccaggcaaaaaagggt, attggccggtcgctgt,
ccaaaatttcctgcgt, ttatacccccttaaat, attatcttgaaaagag, agcacgggggagcgcg,
aatatgggtatttaac, caccaatttagccagg, ggttgtctacaagatc, ccgcacacacttgcct,
aaaatccccccccagg, cgaagtcaccttgagt, tcgccatattttttta, ttccaccattctgcca,
cttaggataactgggg, ggagcgaggcgctcga, ctacgattcttgagca, ttgccggtcgtgcgtc,
aggtttaaccaaccgc, tctttatcaagtctcc, cctcctctgtcccggg, gaggcgccggtgtccg,
ctcctttaacttggac, tttcattggagatgct, ccagcacttaaagtga, ggagaggtaatctagc,
tggatatgggggggtt, cctgcatcccccccct, agccccccataatcaa, gctcagatgcatacct,
ggcaggtaattgttac, atctcatcccacacta, aacagtgccagtgcga, accaccaaattgaaca,
gttgaacctcagactg, gaactgttgcctagtt, aaaatttcctgcgtag, gacccccccccggaag,
ctcactttcttccgta, ccccgaggtacctcgg, atataagctctgaagg, tggatagccccaggcc,
acatatcatcaagcac, ggagcggccagccgtc, agggttcacgggggac, aaaatacctctggggc,
cccctcggtcctaagg, ctcactataaaaaacg, taccggaacattctgc, gcatctaatattgcat,
ctccagcatgtgcacc, caagattccatcccga, ggaaaactgtcatcta, atgcagaattctagtt,
cagcatattggccatg, tcaaaaaaaagcgatg, tgtttcttccgcagta, tccccggggggggaga,
actattcttgcattat, ccgattcccattacct, ttgcccctggtgtcct, gttctgtatgtatatg,
atattgtttttatcga, ttcattgagttcagtt, acatcgcctgctgtga, acttttatgagaatgc,
gggcggtcttcagaac, cctcctaggtctcaat, gctgggaggcggctta, cgtactgtgtacaaaa,
aggacatggattgtcc, gtaaagatagggacaa, gacggccatctctaca, tactgctcacagtgtg,
tattcatgctgtcgct, cttcttaggcgatcgg, gccccccgagtgacct, tacatatagtgactaa,
gccaccttactccctg, aggaggacagttatag, ctgttcgggttatgag, ggcagacacgaataga,
ggcagtggcgcctgcg, gggcagcctgtagtgg, aacatagattaacgta, tggcaaaaaaacggta,
tctcaacatcaaccat, tgggtgcccggcgctg, gatggaagccttagtc, agagttagcatggtga,
tggctattggccagtt, atgtctcagtctagtt, catgagttttttacac, tccacggggggtgcag,
aaggcgtaaagtgtca, aactgcggaggcctag, cagtctgatgctgggt, atacacacgtaaaggg,
ctcgtactgtctgtgg, ccacccgcggtgcctt, ttgttcctcgcgatac, tacataggcctaccca,
ctttcacctcagcgct, tccatagctttgtgtc, tagagcttccagcata, gtctacaaagtgacag,
tcggctcacggaaaac, cggattctcagttgcc, gtacctccatttagga, gagcgagctggaagaa,
atgcctatgctcaaaa, tccgatgttggtaatt, ccctatgagatgtgat, tgtgaaaaaatgcgca,
acgtgttgcttcaaaa, acactgccgagttatt, cggcggctggacattg, gcgtgtgggtgggctg,
agccgaaaaaaacaca, ataccggaatgtccaa, cacttatgtgcttttc, gtcggtggaaagtcaa,
gtccatagaagtgaaa, attgttcggatgatgg, gcgtaggcccccccga, ggaagggtaatggctt,
taaccccactgtacgt, ttcctgccggagagtg, actcggcagtgctcca, ccatggagttactgac,
ccctacggtatcacca, tagggaaacgtatgac, gggatcgaagccggac, accgaaggaagaatga,
ctctgtgttataccgt, agataaaaaaaatgcg, tctgtctttgccacgc, ccctgtaaaagctgca,
tactacacttatatca, aatctcttgacacatc, ccttctctactggccc, aatcccacaagcgccc,
gaggagttaggtagac, gggaactctttgtaga, taggcagagttagact, ttttgtaccccgatgt,
atctgaggatgctgac, gaatgatgcaatgctg, taatggtctgacgctg, agccatttgaccagcc,
agcaactttgggtgac, tggcagggggggtcct, taaaaaaggcagtcct, cggatattgcattatg,
gcaccttgacctcgca, ctaaatcaccgagagc, aaaatgatacgctcta, aaatagactaactgtc,
tgtttcacacaggatg, cagacgagtaggttat, gggttgaacaccagcc, cgtagtccattagagc,
aacaatagcccttgtt, cctccgagccaagtat, aaacaatggtatatgg, tgtccccccaccggaa,
gaaaaccctcacatta, tgggggggttagccct, agcattgcacaagtat, cccttgcagagtaagt,
ctcggataagatgctt, atgcattacttcaccc, aaatgaggctgcgagc, atacctgcgactgagg,
atgatgttccccatga, aaacagtaatgggcca, cgcaccgtgagattat, ggtaaaaaaaaggctt,
aaatggttaccgcaaa, gaaactgtggggggta, gtccacgtccatgagt, gtggtgtcaggcacat,
tgtcgcaaccttcccc, aatggacttgctgacg, ggggcggtgctcgtct, ttaacccgggaggcat,
aggaggaatccctggc, aatttttaacgtgtca, tctgtgtataccttgc, tctttccattaagact,
gtgaatactgtcgaca, caagttgggtgaatcc, aaactatgatatcagt, cacagctcgcttctgt,
gtctgattcttgcact, ggaccaacctccatga, gtagggggggggacgc, gttgagcacgggggag,
gaatttggcattatgt, ggggacaaaaaaaacg, cctgaaatcgaggtgg, cgttttttttactagc,
tatatgccagttatga, catggcttaacctcgt, tatttttactcagagg, accttggagctcctta,
cctcgggatgcttagg, tggggcatttacatgc, gagttagactctgtcg, aaattaaacggatgat,
ccccccttccgggagg, gtatccatattgccat, atgttttttttcgtcc, accattatttggatta,
aaactctttcacgagc, taagtgttccagcttt, cctcccccacgacaaa, catctgtgggatatca,
attcccctctgcttaa, cctattaagttgcaat, actcacttaaagcatc, ggtacatgcactgaga,
gtcccccctgctgcta, agtgagttagactccg, atgagacctgtaatac, agagtggagggggggg,
cgacaacataatttta, gatgtatcatggctgt, aaactaagtcttatag, gcttaaggagccgggg,
atgaggcttattagtt, ctaggcaagacttgag, acaagctacagcgttt, tagttgcagttagtag,
tgtgtttgaccaggac, tattccaacacactaa, ggtgaatggtgagggg, accgagggggggggaa,
acctccgcatggtgcg, ttattgatcttagtat, cgtgttacataggctg, accaggcaacctatga,
ctgcaaaacggtttat, cgccaccagtatgcaa, gatactcccatgttgg, tacggttaaaccccgt,
ctcgggggcgaaaaat, gacgagccagccaggc, ggcaacagtgcattat, acctggcagtctcttg,
acatgcttcggtgtgg, caccccccccacaata, tcgatatttactttaa, caataataacacgaca,
cgaagcattgtgcatt, tgttcagtagggatca, ggggtagtttgagaaa, ggaatcggctcccaaa,
tagggcatatcttctc, tattggtagtaggttt, cataatagctaggcta, gaacggggggggaaaa,
ccccgaacaatgaaaa, ctggggcagttgtaat, atatgcatttctcgga, cctcagcttcacatag,
ggggggctctccccgc, gaagtcttatgtagtc, catatgttttttttac, tagagtttataaagtg,
ggagtgaatgccctgg, tcttagtacgaatagt, gagctaaggggggaca, ttattactgtaatgac,
acccactacagcatgt, tctgcttgctacaaac, agtcacaaatgcttgg, ccccgtcttttttttg,
ggtgtgttaaaccaca, gcgggctaagccctcg, tatgcccacgttacct, ttgtgcatgcgtcact,
atgccctgcccgtaag, gggagaacgagagaaa, tctccctaaacccaac, gatgacatcgcctctg,
gtagactgtgcggtct, gctcaggatcttcgcc, ctggttgggatgttgg, ctttccaccagcttaa,
gaagaaattcagcggt, tgagagaggtacgtaa, agagcccttttttgtc, tgtaggcccactccat,
gctacgccggcggctg, gtccatgggcaacgca, gttcgggggttaccgc, tgttcttaacgaaaaa,
ctgtaggcccaaccta, gggcgagggaaaccac, ttatttttccgatttc, ttagattgccccactg,
gtttagcactccccat, acagcctctattagtg, catggtcttatgcact, cactaaaagacctatt,
catgcccccccctccc, atgagaacacccgggt, ccgagactgagtagac, aatacttcatagcttc,
gttacttaagactata, agttttaggagggtag, ccacaattttaagcat, ctttggctgatatcct,
aaaacgggtgctcctc, gcgtggactctggtca, agtcagcaatgccgcc, tgccaatttttggatc,
ggacattgaacccatt, gcgttgcggcgagggg, tttatgctagaagggt, agtttttagatgccgt,
gtaataatttcgtatt, atcatgagttctggtt, atggttgggggggtct, tacaagtgtgctctac,
gtgcaattaacatggg, gtgacttcccggtcct, ccctctccgtccttag, gccagcgtcggccccg,
ggtatatacatgggcg, ggtggtagtagcccag, taatgctttttttcgg, gtaggaatcacctcct,
agctctcgtcttccct, ggctctcacacggtgc, aactgttgcaaattgc, gcttccgtgcacatct,
tgttcaaaaaaaacct, cagagctttaagatta, ccgggcagctcgtgct, agaagcatagatgtta,
ggatccctcatcagac, ggatgaatattatgcc, tatgagacacttataa, tcacttcgtgggggtt,
cggggtgcggaggggc, atgtcgcaaccttccc, cgttagtaaaaaaaat, agagggcgctagggcg,
agatccaacactgttc, cttcttgttgaccaat, ctgccgctgctttatg, aagagttccggttcag,
ttttgcactcggagag, aagagacataagggca, agtgccggaacagtta, ctcccgctttccatcg,
gcgggtctccgactgt, ctagtgcgatcacctg, tggtaccaggcctgat, agtaccacccaccacg,
gtatactgttcgggtt, agtactttgtcggcaa, gcattgcttgaacctt, tcttgactttaactta,
gtcaccgttagcatct, ggcgtaggttccagtg, acacagcatcatattt, ctaagtggcctagacg,
ctctttgccatcactc, atcaacttattttcgt, tcattcaccgcactgg, ctaagtcttcacttga,
ggtgaaatgattccaa, cttactaagcactgag, ggggcaattttaaaac, accgagaccatgatat,
agttagacccaatcac, acagcataattagttt, gtgcagcctagctgcg, ctctttgttaattagg,
atacaacctttctttc, catacagggcttcacc, atctttggcacttaat, cctagtttcaactaga,
cgtccctttctccctt, ggcccgcaggaatgta, acttccctacagacgt, ctccatctgccggcct,
attccctacggtatca, tgagtgcttcactcgc, gcagagatcacgggtg, atctaatcggctacta,
taggggggggtagaag, tgttagttcattctag, ctgaggggtaatttta, ctcaacgaaactgcaa,
gacttggcgggggggg, cttgtggatggatccc, tatatgagggttatct, taacgtttgactgaat,
ccggaagtgacgatac, cttggctgtcccgccc, actatagaggaagatt, ctttgccccccttctt,
taccatctagagtaat, tactttcacaccttaa, accctttttttaggta, tgcgaagaaattcagc,
ccctataagggtgaac, cggttcccttcaggtg, gggtcctcgtgggcct, tgacttttgaaggaat,
acgctaccggccttgc, aggaatgcccttctga, agctccgatgacacat, cctataacctcatgaa,
catctgacacaccgta, ggtgccttcctccgct, attgatgtatggtatt, ctgtgaggcatagggt,
tatgcttaattaggga, gaaagccctaccttag, gcttaggggtgtgcag, tcttacgggcttagtt,
cgaaaaaaaaccacac, ggcgcggtgtcgcacg, tcctaagccagacgct, atgggtgatgcaatat,
catatcttacttagaa, gacccccccccaacat, gtcaagctgggcgagg, cgcttaacataaagtg,
gatgggattccaccgt, ggcgaccctaaggctg, caccgtagtccattag, gaagtgtgggggggtc,
cggcccgcgctaggca, tgagatgatttgtgca, gactctctcgactgac, ggcaatgtgggccctc,
taggatatgtgtatcc, gtatgatcctgatgtc, tctgggactgtatgca, tcctcctatcgacttg,
ctgttcaatgtgtgac, cgcccttccgcactgc, tcagacatttgtccct, gcccggtctcttcagt,
cttcctccgggtcttg, tttgtgctcattcact, cccactaccgtatctg, ttatcaactacaagat,
ggctaggctttgttcc, gcttaatctggatgtg, cgacaaaaaaaaggag, tccgtctcgccttggg,
ccagtgttgtatcaga, ccttagaaattgggga, gcctgtgcggtgtcta, caagggcataggccca,
agaactaactgtcatg, ttccaaaaaaggacta, attacaaccttaaaca, cctttaactctcagtc,
atacccccatatcacc, gatgcgtcatgttcag, cgtttaaaaaaaagag, cggttaatacggcaac,
ccgagagaaggaggca, acccatactttagctg, taataagtagcctaac, accaataagttggtct,
agaccccgagaactat, tcggggggcgcatata, tgagttgtgcttcaac, tacggtatcaccaggg,
ggtgaacaggccctaa, gactgagtctcggtcg, aagttcatctaatcta, gcgtcagttataagcc,
ataaaattctgcacca, cctctgctaaaacctc, ttcttgcactgactaa, ctcggcctcaagttat,
acccaataacccacaa, ccttcacgctgggctg, gcctgtgcttaccctt, ttcgaatgtccattta,
caggaggagttctcgc, gtcccccccccgaaaa, atgggctctaactaaa, tggcgagtccaacgtt,
gtttcccctactcatc, taggactagctggaag, gcacactgaacagggc, catgggcctcggcctt,
ggctaggcccacccac, cgtgccttccgggagg, gataattcttgtcatg, tggttgatcaatggtg,
atttattgcttaccaa, aactcgtcctttgtaa, gttctcttaattgcga, tgcgcactgctggatg,
aattaaaggtggtact, tagtcccccacttatg, aggaacttttgcactc, taggataatgggcgtg,
cagaactgtccacgtt, aggccccttctagcat, gagagcgtcttcttac, tgttagtccctttgct,
tgcccattcaacccag, tctctccatgttatgt, ggccattctttctaac, cagacctaagcaagtg,
accggggtctcgcttt, gttttagcattaaggg, tcccattctacgggtg, tgagcaacgccgaaga,
caatgtttgtaattag, tccttatgttgccact, atattgaaccccccca, gaagccagataactgc,
ggcgatgctcagggct, caccgcgcattccgcg, tgcgacaaaaaaaggc, aaggggctggtcttat,
cagcgggcggcggacc, tccccaccaacgattc, agagccctcggttggt, caaggtgatgacacag,
cacgtaaatgtggacc, acaactccagattcgc, agccagtcccattctt, tgtcttgttgttgtgt,
cttaacgccacatgat, ctgaacagggcagttg, actgcctgagccttaa, cccacccatcacgtag,
tggggggggagtgcat, attatagtgtattacc, taaacgcccttatttt, atgtgttagctccaaa,
ctcagcgacacgagcg, gttattcctagattct, agatcttagagaccct, ctggtgcgttatgaag,
ctgccgctagcgcggg, aatattatcaccctat, gcgagcacagcccctg, tagtgccatggtggca,
ctgtatcggttttaga, gtggatgggctgtggt, gtgcgccgcgggacac, aagaggactgtagggg,
cttccagtagatcgaa, aatggcgatatttata, ccttatgagcaaaaaa, tcctattgtgacagtg,
caacttatgaatccag, tttagatcccctcagg, ctaccctagcttctag, gatgcagcataccagt,
gacgtatagacatacg, cctcccctgttattca, gggaggatcgctgtaa, caacagagaaggttga,
tgaagcgtgccagagg, cacccctagatggtgg, tggcttgcgccaactg, ctattagtctggacaa,
ataaaagacgatatta, gagatactgaactgac, gagtgagctccacccc, gaggctgattaatgtc,
cttgcgtggtgctcca, gggtaccctctgctgt, ttcattccgatattat, aaacgtgcatttttta,
ttattgatgcagagag, taacatttgcgtactt, tgcgattgaagcagac, ctcaccaccactaagc,
tttgtcagcgtgtggc, tagatacttggtctta, tgggcacaagatctta, gctgattaaatcaggt,
cctgactgagaactga, atgattcgactccgag, ctgcttcgctgctgtg, tattcgggggggggtc,
ctgaggaaatccattc, gaaattacccccccca, ggccttgctttacggg, cgaagcagagctaggc,
atcatgttattgtgcc, taaatacgtgccatgg, acactctggtaggccg, tttgttcctcgcgata,
ggcaagttattagggg, tagtatcccccccccg, catataaatagcgaac, agtgtccttacatagt,
ctaacctggttatgac, taggcaattctcagtg, acatgagcccccgaat, cagtaggggcttactg,
cacagagcttaaagca, ggagcgtctgcggaag, catgcttcttactgac, aatgatatgtctgcct,
gttactctgtgtggga, acctgtttatctgagc, cccccctttcccctgg, ggagctcttattgcct,
cccgagaactatgctg, ggataacttgatgttg, caggatcttgtgctca, tggggcttccccagcg,
tgttccgcggccgtct, tggaaatagcttgctc, tatcctcagggggact, agcaatcaccgtcttt,
ttcgaggtgctaagtc, atccagaactgcaaca, aaaaacgagagtatac, tcatgtggcagctgat,
ctactcctcttagctc, gagccgttggctccgt, ccccctgcctcaaaac, aaacaagaagcgtgtg,
aacgcattgttcggat, tatagagtgggtgcct, gatcagcgccccgtcc, tacacagcgcagggag,
gttatggtgacgggga, gtaaacttatggtcac, ttctggtgaagtgtaa, taggggggatagcatt,
ctactctaggtgcaca, cacactaggttataaa, aaacgggcatgcctgt, atgaataacctggata,
gagtctgaaacgagct, ttaatactagcaactg, ctcaccctgctcataa, cagacttgtgtcaaac,
agctccgcgcggggct, aaagcatctgaggacc, gaccccctttttttta, aagaactcttaccgac,
tgaccttttttaatcc, tactggcaacagcatc, tcagctttgagggaat, ggtcagagacaagacc,
ccggcgtgcagtggct, aacctttgaaaggttg, tgggggagtaggttct, agatagcgtagctcga,
acgataagctctgctg, tctgggtatgatgaga, gcgggcgctgcgcgct, ctaacatcactgctct,
tgctcagtcagcgttg, tatagtccacgagaac, agggcgatctgaggat, gtcggctattttttta,
cgcacaatacgccegg, attacagcatcacgaa, atagcaccataacata, gtgggcgaataatgag,
gttctgaaattacgtt, gtcccgctgcccttag, ttgtatggctaaggct, ccactgtcttcacgct,
acccaagctctagaga, agcaccagtcagaccc, tgagagtctatgttct, tggacgggctgagggg,
cgctacagaaaaaaat, gggctgttgtggaaga, gccggagtagacaaat, gtagtaacttctgtgt,
caatctattcgtgcac, aggtgcggatgggaac, agaaattggagaatcg, gcacataagttacata,
aacctgaacacacatc, tgaagtcattggtgga, gtctgcacaagactgt, tgccggagtagacaaa,
ggcccagactccagtt, taccttttgggggggt, accttagagttaaact, atgccgtggtcaaagt,
gccccccctgtgacat, gtgttcagtagggatc, atgaaaatggttttcg, cctacttccactcacc,
ttaaggggggggtgga, tgcttattgaaagaga, gtaggggggaaaagca, acccacgatgaacgaa,
tagtatttgcacgtca, ctaggcagcaggacat, actctaattgtatctc, gtgtcccggtgattct,
tgtgaatggggtaatg, cacagatttatgtgcc, acccataagctcttta, atatttcgctttcatt,
ctagctttttgattat, catcttttaatcccca, ctattgatcttaatag, gttctaggttttagta,
caataggatacgggac, gtgtagttgctctttt, gagattagacatacag, aagccaaaaaaaggtg,
gtcgaaatttttttgc, tattaagaattcggcc, cccgccagcgcattac, tgtcaggtgtggaatg,
tattaacattgcccgc, aggcaatcaagccatt, gaattaacatgctaga, tgatattgtgctgggc,
atggagagcgtcttct, catagagaggctttga, ctggcgatgctgagat, taatccgcccgcctga,
ttccttcccccacgaa, tttgcgcagaaattca, ccctaaggctgcggct, tgtgcagcccgtccgg,
ttcccttttccgtttc, caaagcttccgcactc, atctgtagttactcaa, atctgtagtcccttgc,
acaagactaataggta, cagatagagccattgt, tagctaccacgtgctt, attgtgctaagatcgt,
cacaagcgcccatttc, accaacactgagatac, gtccacaatcaggggc, cctgttcccaaacacg,
gtatgtcatagcatca, tgtgaaacaatcttgc, aacacaacatgtctga, gttgcctagttcccag,
ttttctttatctcgga, acacgattttttatca, ggacaattagcgcctg, agcttgcaggcatgcc,
aagcggactgtacatg, ttgacagtccacggct, aggaaaacggtattaa, cgacagccttaatgtt,
ttattggggaagcact, atgcttaggaggggcc, tgcacaaccttgtaga, aacgtatgaagcacaa,
gtagaattgcttgcat, aactaggaactgatgg, ctacaagaaaacttat, ttacaaattgctgccc,
tggtgcgttatgaagt, tctcttagtacccaag, atggtccccgccgagg, ttagggtgtctagaaa,
ttgatatagccagaag, cagcatacgtcagctt, gcaccaaaacaaaatg, tgagcaccgtgcatcg,
tgaaaaaaaatagcgc, ttgtgacggcagcagt, tgagccccgcacacac, agaaggtggcctggcg,
ttacccggataatagt, cagtgcactggtcttc, atcgtacagtgggctg, catttttccccgtgac,
aagaatttgggtaatc, cgcattgacctttccc, tctagtggagaggtca, atctccttacctcgtt,
gttgtaatcctattct, tgatgtccatgagttg, gatcccctgcagtaca, tattacagtgcgcccc,
tctacaatccgaaata, acggtagttataaacc, ttcgtctaactctaac, gccaggttggccctct,
cgaaactctctcagaa, atagtggaagtgtagt, actgtgtaccgtctgc, ggttccccagccgagc,
atagcattttaccccg, cccgaaaaacagaaac, cacttagtgcaacgct, actagacggtttttta,
ttgtggagcaggattg, agcgtagcgaggcgcg, cacattaggctgtggt, tgtttagtctatggcc,
cctctatgctgacaat, cgaagttttcaggggc, atatattaagggttca, ggaattcactttccga,
cggagcggccagccgt, ttcagcacaccaaggg, acagactgtttgctgc, attacagctataccct,
gagctgggaatttcgt, tgagctatagagctgg, acttcgcatttgctag, tggtgccattagacca,
ttaatgtgaagtcaaa, accgatgaaaggagca, gtacgtgtttaccaga, ggaacggatcctgggg,
atgcacattgcatcat, atgtcagcgattggct, aatgcatccccccaac, ggagcatacgaagttc,
tgacatagttagactc, aatttaggggctgatt, tactatccttgtattg, tgaggggtaattttat,
cttagtagctagcatt, acatgactcttgtagc, cgcatgggcttctggt, gtatctgtctgtccta,
gcagggcatagcttaa, tgatgcagtaaacagg, tccgcaggagagcagg, tgattattcctttagc,
cccacctgcccgcatg, aaatccccccccaggc, caggggtttaaagcca, atgcgcagtgtaacta,
ttaggctggacgtcta, aatatatagaagccct, ctggtttgtaaaaatc, taggcacttaaaacac,
ggctagattaaacaga, cacttgatctaattaa, atttatctaatcacag, aaaaaagcacatcgga,
ttaaaacgaccgatga, aggcacttgccgcagt, atcataaaaccagagt, tcccttggtatcttaa,
tgatgcctggtatggt, tccgatctgccctcgc, gtccacttcagtggat, ccctaattctgcagat,
gggtagaaaaaaaacg, agtctgcacatatgat, agcaaactccattacc, gacattacctactacg,
cctttttttgatgaac, aggaggagttctcgcg, tctaaaactagtaaac, taaaagagcccctatt,
ggtctcacaattattt, gtgtcgccgctcagca, tgttagccatgttaat, atcccccaactgaaac,
accaccagtgccggaa, ttgtttttatatacga, tagcctttacagcaca, gaggacttctctactg,
cccagcgctccccaat, ccggtagcgtgaagcc, cttgtaacttagatct, ggggtttttttaggat,
ttaagagtgctgcata, tgcccccccgggatta, tgaggcggccatgctt, gatgggctctatggga,
tttaccccttggaaca, ccttgtgaaccgccct, cccctaattatgcagg, aaatgtaaaactccgg,
ttatagggtcaagacc, tcaaagttctcctttg, ggctggcaggtaactt, cgactcccctgcagta,
tgattacagattaggg, cttacacagaagctgt, tgatgaaggggatgct, caaggagagtatcttt,
cttgctagaagcacat, tctgtcgttgcccatg, agtaggggcagcctgg, tcaaaccttctgtggg,
tgccactgtatcggtt, ttagttcctccaaaac, tggtgattagatggta, gtcaataattatatga,
gcgaaaaaatatttat, acccccacctgaatac, ccagcagcggcgggag, ctgaaaagcgggattt,
gctgttaggattagat, gaggagcattgctgga, cgtacccaccgcctcc, gcttgcggtgagctta,
tattattgagtctcga, aagaattgtgcgcaag, tgcccatcgggaaggc, gagatgaacattgtag,
atagactgagagcagt, aatttaaaaccgaatt, cgtattcaaagactat, gatcaagtccggcagg,
aggctactgccttgtg, ccagaactaggcagac, caacgctgcacagttc, tcccccccccagtcca,
catcacaggggggagc, cctcagagcccccccc, ggcaaagggcgtgtgt, gattaactgctcatga,
caatcccggccccttg, agcctgtcttctgtga, agtgccttgggtgtgg, aagttcagttcacagc,
ccttggcttcaggtac, aataataaccttcacg, ttactcacctgggtga, ttcagcctcacctggt,
cagttcctaaatgctt, aactttggtttcaatc, tcttaccttggttcaa, gtcttcgtagatccag,
ggtcggctaatatttg, gtcacggaaaaaattt, tttattacgctgagaa, aaaaccgaagaaagga,
attggcctttttaagt, gcacctgtaccctaag, tttttgaggattactc, aggaaatcgaaaccgt,
gttcagaagggtcata, taatatgtaaatttcg, gaattacttgcattcc, tatcgtggggggtggg,
ttttccgaagcaaacc, aatattaggcaggatg, ctcttcacacgggacc, aggactacaattaaca,
gccacatttcttgctc, aaacagtctcccatag, tgtgcacccccccagc, gaggccggcgagttaa,
tgcttttgactgcaga, gcttccgcactcttct, gaggactctagagagt, taggatcatcacaacc,
acttaatactgaatag, catcttttattcaggc, aattctaggcctggcg, ttaggaggctcattaa,
aatcaacaatctcatc, actggacatccgccgc, actttttttagcctaa, ttaaggacatgggagt,
aagtgtaaacctgttc, ggagatggcaggtacc, aggccgccccccccca, ctgttgactaaggtag,
ctcacctccactgccg, caccattgtgaattct, ctaacaagttcgtaag, gctccaaaaaaagatt,
ctgctgcttatgaggc, ttacatcccagggatt, acacaccacccttagc, agatgtggatactaga,
cggagagcatgccgga, ccccttctgtggggtg, cacatggctggttcag, cttgctggtaaaagaa,
ggtcagtgtttgtccg, ccttggctgaatagcc, gcaatataggcgtgca, ttctgggccactctta,
gaagcttcccccccca, ttaaaggcagttatcg, ccgagtgacctcgggg, ccgctgcaatgcccgg,
ctacccccccatacct, ggcttaacctcgtctc, ttcgctgctgtgtcct, ctccaaggtcttacca,
cctggaaggttgtggt, actcatgaataccccg, atgacagagtgcatct, attaagtgggtaggta,
actttcgtggggggtg, cctgtgctgctagcac, ttggtccctaaccccc, gtctgcaattctacga,
ttacagcatcacgaac, gacagggggagaacag, gagacggccatctcta, ccccggccactctggg,
gttgtacaggttggat, gggaggcagggggggg, tcatgactgggcccat, cttactatggactccc,
ttacccagctcgattt, tgactatgattgaaat, tacatccccatatatc, attctggcacgtttat,
ttgctagtcatttaga, cggtgagtcatctagc, actcggcggcagctgt, cccacccagtctaaac,
tccaaaagctctttcg, gagatccaggctcccc, gcggctgtatgtagaa, cgatgctcagggcaac,
ctgtaagggaacatat, gaactgagcataaaac, gaggtgatggggttca, ccttgccgggccaaca,
aggcacccctgcagtt, tggatgctccgcaatg, catgatattactgagt, ccgacatggctaaacc,
ggtgagcactttgtag, gcgactgtgtcatagt, tctgtcattcccccct, gtatcactacatattc,
tgagtcccagtctaac, acatggtccataaacc, ctggccatatatcttt, agggagggggggggct,
gtgttacccgcacatc, agagaacgaatgagag, tcaagatagcgtagct, tttcttctgggtggtt,
ctttaatgtttctcga, tgttattcaattacag, caacataggctggaca, acagtataaccgttat,
agtagtagaagctgca, ctaccacgtgcttcta, aaccctcctaaatttc, ctgccctcgcctgttt,
gcctgaattaagttgt, acactgaaactcaacc, tctcagtcctatggca, gcaaaaaaacggaact,
ctgctgaggggcatgt, gttttggcacaaagtc, ccctgggacgggggtt, acttagagtccctctc,
acagtacggtgatttt, tcttatcacaattctg, tctcaggggcaaaagc, gtactatattagaaca,
ctgtaatatagtacat, gcagacaaacccactc, ccccctccctagtgtc, gatccaataccccact,
aatgaagcttaattgt, ttaagtgcagtgacgt, cttaatcaatgactgt, cggtggctccatcccc,
atatgatgtgagtaat, cagctagccaagatct, cacagtcattgcggct, tatggggacgtcgaag,
gccctaagcaacttat, cccgcccctacattgc, ccagctacgccggcgg, ttctagactgatccac,
tgtgcttaagacaccc, ctgtaaccgcttaccc, tacaacgcgccccccc, catgagcaacgccgaa,
tgattaatagaacagt, ccatcgcgtggtgaca, gtgccggatccctgag, gctggactggtggtgt,
aagaggtcacctccct, tgtcaaactcgtggcc, ccccctgagaaccctc, tatcgtcaaatatccc,
ggagccaggaggttag, taagccagtgtcgccg, agatctcgtggcccga, ttatggtgacggggag,
tgtcggtgggggggat, ccagtggggaatttta, ttgtgtgacaggcctg, cagttcgaaatggaat,
gtggtgctcagagatc, cttctgggcgtgagcc, agatatgcgggatgct, tgggtgcaccctcaaa,
ctatcttttttttggc, tgcggaggacaaacat, cgatgatgcagaaagg, gggcatgggggggaaa,
accgcgcattccgcgc, aattaactatttccac, gttatactatgtcttg, aaatattgaagatgtc,
gaaagcagcagactac, gtgctgccttagcagg, aaaagccataatcgtg, tacagatttccaccac,
ctactggacggggcgg, agctacgcttcttttt, tgcgtgcatcttgcgc, ttggttggtgatagaa,
tatgatgtaagcctgt, atctgttgtcaagtga, tgcgggcatctatgtc, ctagagttagtagaag,
cccggcatccttgagg, cggctgggactccatg, aggcatagtaacttcc, acccacctttcatagg,
agggtacactgactct, cccaggaatattcatg, gattccatctcagcgg, cagcctcattggtgta,
gcaacttatgtctgtt, cgatttgcttaagatg, tctgactgcggactgc, tttgcgtacagtaaaa,
cgcgccctgccaatta, cccccacagccttggt, ttaaggttattggctt, aaaacacggtttttta,
gtgtagtctcatcaca, atgaactctaatggta, tacacctcaacatttc, actggggaggacgaaa,
tcgtatttcaacatta, gaaatttggggggggt, cttttatcgtgacgtc, gatgactgccatctgg,
gtccttatgttccttc, aatctgacgtttttgc, gaattcgacccagggt, gcgagtgtttcttttg,
ggcagtctggatccta, gcacgcagaataattt, gcagggccgcgttgcg, tacaggcagcggatga,
aagctttttggctaac, ggacactctaagttag, ttatgcattttgctac, aaaaggctctcctgta,
taagggggggggtgtt, tccatagctgccgacc, atacttactaaacaac, attacgtctgtggtaa,
ccatagctgccgaccc, atctgccccccccggc, ggagtcactaacaaga, tgtggtgttcgtcttc,
ctccacaagagtgggc, cagatacctgtcaatg, gccttaacttgattgg, ctctttcgattgtaaa,
gggttataaccttgta, ctatgggggtatcatt, agtgtatttctccaag, ggggtacactgcaaag,
gacatgcccccccctc, aagcccacctgggatt, gcgagaccacacttcc, atcacttgagcccgat,
ttgggaaaggcccggc, cacccaccgatgtgtc, gccagttggttgaact, ttagaacgcacaactt,
ttgtgacgttaagtcc, taagatctgtttcccc, ttacccactcataagc, gctaagtgttccagct,
agaccccttgccaaca, taagaatgtcgcaacc, cgtacataattttttg, actagggttcctggga,
tatctcttaccaggcc, ctccttcgtgcctttt, ataaaaaagggcctga, gaggtgaaaccctgct,
gggactgtaccttatc, agccacaagacgagtt, cacagtagtagtggat, gttcaaagctaccaca,
catggcattcggccag, ttggttggctttggtt, agataatgccccaggg, tgctcttcgagggacc,
agccgttggctccgtc, tgtcttaagtgttgga, ctatgttttttttacg, gcgatgaggattaccc,
ttggccttgtccgagc, cacagcgtcgaaagct, cttagccccctacacc, tgtggcctcttgcttg,
aactaattctggcacg, ccatcttctaaccctg, caatggtataggattc, tcgccgctcagcattc,
gcccggtgggcgaagg, ggtcaggtgagggact, tcctccagaggtggtc, atcctgttccttaaac,
aagtgccctacaattc, gtcccgcgaggcctag, actctccctctttact, tgtggcctgattatgc,
gcctatcaatttggcc, gaactatattaacatc, ccttaattcaggagtt, cgaggacatgatctct,
gagtccatgtgctcct, tcgacccaatattttt, atttcgaaatcatgct, gacacgtctgtaatct,
acttgtggagcataca, agcaatgtaagactga, ccacaggggaagcttc, ttctttaatcgttctc,
ccgcgaggcgtaggtt, agtcccctttttttta, ccaatgaaaagcccgg, aggggcttaacacctt,
gggcgctgcgcgctgg, taatactctctactaa, acccgctctagaaggg, agtacacaatcatact,
ctttacccacatttca, agatcttagaacgggc, gtagttaactaatggc, ggaaggggtagccggg,
tcataactgaaaatgg, actcctataatcccgc, agagtaaaaaataccc, agctcttatttgggga,
caacactccgtctaac, tctcagccttagaggc, tggaacaaagggctaa, cataagagttcctgcc,
aaatatgacgcaaatg, ttttcatttggatctc, atcatgttgcatgtac, actactgcaatattac,
tagcctttaggcactg, aaaactctgtgactga, tgatagttcaacataa, aatccctttcatgtcc,
ttgagtacggaactca, tgtgggtcccaactgc, aacgaatatatctatt, cgtttgtacttatgta,
tgctggaggccgcgca, tacatgggtcatcaca, gagtctgacgctgtca, atcccggggggagacg,
aatctattcaaaggtt, ttgtgctctcttttca, tggcttaggcgggtgg, aaccttggagctcctt,
gactaacataaggcac, acccgctgaaggattg, cttcttgactgcttaa, tgtccaaaaaaaggct,
tggtccacatatgtgg, acttatgggtgcagca, tagctcccccccccgc, gtcatggttctcctta,
gtaacatccaggtatt, ggaggtaggggggggg, ccctggaagcagccgc, gcatcactggtgtgta,
tgcaagtgcaggatgc, attgactccatttggt, tgaagtggacttttat, agattagatctgggtg,
tgtatggagatcaaaa, cgcaccaaaaaaaagg, cagagttcctcccctg, ccagttactcgtaagg,
ttttagtagggatctt, ccttctgcagattagt, ggatggccttggagtt, aacgtggtggaatggc,
agtgggctgacttaga, cgggaggcataggttt, gattataacacgaata, aacgaaaaaaaagtat,
agtatattaaccttaa, tctcatacctctggcc, gacgcccggccccgac, tatcctgctgaaatat,
tacatcttggaatgaa, tagattgttgccttac, acgccaagtgccctac, gaggaacttacagtca,
aagggttggaaatggt, taacgggggatggatg, agtcgcttttttttcc, cagcccaattaatttg,
ttgcgccggcgcagtg, ctggctcggcggctgg, ccctttttttgaggtg, ccttagaggtgctgat,
tcctgccccccccgaa, ataaccccgttaaaag, ttgcaatcatgatctt, tatgaggatataccac,
acgatgaacgaaaatg, cctggggcatgaggcg, ccgaggaggttacatt, agttgcgttatttttt,
accccttaggtcaaga, cgttcagaattcttac, tttccaccagcttaat, ctttcttgaactgctt,
cacatcctccttccgt, ctcgtaaaaaaaccca, tatggtacgtaccatg, cctcgagaacttaaaa,
aaacggggggggaatt, aataattgtaaacggt, gaggctgctaggcggg, gaggctcggggctaga,
ggggcttgtagtaacc, cataaaaaaaaccatt, gatgacttaacgccac, ctcgtcagactccaaa,
aagtagactggcatga, atgagcgtagtgacta, tggccatttgctgata, gtgtagaacgtagact,
gtagcttctatttcac, tcgaatggtaactcct, tgccgtgtggctgaga, gccatgttctgcaagc,
tggcgtcatattgctg, ttcttatagtattcac, catcttgtctgcactt, cattagcccaaaatgc,
gaggctaaaccaactg, caggtgtgatccgccg, tatcctggttagcact, agcactcgtgtactat,
tggataccatgctgga, acctttgggtctgcca, gcttttttttgccggg, aggattacagccccat,
agaaaactagcatgac, cgtcacgctttttttt, acgctacttaaaatgt, tgcaataacttaactt,
tggcagtgtaaatgcc, ctgtcttgttgttgtg, gaatctgcctagctca, cgctacagttagctgt,
tgctcggtttttttaa, acagtaacaaggttcc, aacaacattaggtatg, actacaggcagcggat,
ggctgccgctagcgcg, cttttagtttctccca, cctgcgtctgtccagt, aaacgaagcccatttt,
tattgggggggtgaat, gtgtcgttttctttgg, acggcgcaccgtgaga, atgatggccccgtgtg,
accttagtttgtaact, cccgaacttgaaaata, gcgttttgttgtttat, gactgtgcggtctggg,
gtgccggtttgtactg, aacgtaggagagtggc, agtagaaaaaaatcgt, tggtttccaacgccca,
tattgcccacaactga, atggggcttagaacag, ctcatctacaatacca, tcccctaaattagaga,
tcacatggggactgct, ctgcgtgactgccttt, ccattcagtaactaac, ggaggatcgctgtaac,
tgcaaaaaaaggacct, tctatgagagttagaa, ctccttagagccattc, ctaaaaaaaacgtaga,
ggtcctacttgtgctg, acaaatttgagtattc, cattagaatcgtttga, ataccattgagcctta,
gaggtaccctctccca, tagatggggggggaag, acctgtgaggttctag, ccccccgaggtacctc,
ctgctgtgaggttagg, tgcctcactaaacata, ggaattttcccaagtt, aacacccgggtggagg,
ttataccaaagtctgg, catagccaccaagatt, tatagctcaaaaggcc, ggtgtggccccccccc,
gtaggcccccccgagc, atgctacagtcctcat, gtcaactgagggttgt, acacagtcgttttcag,
acaaaggtgagggggg, cagtgagcataatctt, gaatgactggactcga, ttgggcacaaccacaa,
cttaaagtttgtgctc, acagagtcagctgtaa, tcccctcgaagaaaag, gctgggatcgaagccg,
tttaggggggaaccta, ccccgaatgaacccct, gatcttggattaccgc, gtatgggggggtaggg,
taggttgaccgattca, ctcacgttttttgcct, ccccttgtcagggcac, tgaaaaccattactac,
aagccccccccacttt, gagcccgataggcgga, ctctccgggtgtcctt, gcccgctgcaatgccc,
tgccagcgggcgctgc, ggccggcgagttaata, gatgacgggcgtggga, cacataagctcggtca,
atttttgacttatcag, agaaaaaacggaccca, ctgacgtggttgtggg, gtggtgaagctttatg,
gccttagggaacaaag, actgcctccatctcgg, agagctcgatgtttgc, cagcatcacgaacagt,
gtgaagatttcatccc, ctcttcaagcccatta, ttccgcgcagtctctg, atcagatgatgccgga,
cactgtatcggtttta, tctggtttttttggca, cgggctcctttttttg, ggaatttccactccca,
ttgggggggaggcacg, ctgacttcaggcgatg, atcacaccaccagatg, aagccgaggtgagcaa,
aagcaacttatagcta, ctctttgggctcggtg, aaatgatacgctctaa, taggctacctttatca,
cacacactggagtgta, tggcccaccatgaaga, gacagggaccttttgc, cccatcctgtttcgag,
acccaacgaggctact, cttttgttatcaacag, atgctcgtgatagtgc, gatgtgtccattcgag,
tgactcaattaaccca, tgagtctcaagacaac, cagcactgcagtacgt, aactgttaactgtctc,
gaaaatcatttcacgc, actatcctgtgcttca, tgagagtagctaatag, accagtatctcggcac,
cttctgggccctgaac, cgtatagacatacgtc, cttatcggaggacaga, atgtcccagagatagg,
attccatagagtgaat, gaaccttttttagaac, gcgagattcctcagga, tgtctccttcgaatgt,
actctcacatcttaaa, gatgagtgtccggctc, tgggcgtaggcccccc, cgtttcagtttatcct,
acaggatcctgtggct, tccaaaaatcgccaag, atgtagactcctagat, gttggctcctggaatg,
gcgctggcagggttca, actaggtcgggagatt, ccttactctttagtat, tctggaggcgccgttc,
gaaggccgtactgttt, gacgctggacccacgg, aaaaggccgaaaagga, tatcttaaaaaaaacg,
ttgttaacccaggctt, gaataccataaagcct, ttagggattggttcca, ttatattgtgaaagcc,
tgatgttgctgcaatc, gtttgggtgtgtccta, tttgcccaatagatag, tatgtggaaacttgga,
ccaaaagtatgtccca, tcaccgcatgacgagg, ccagcctcgagaatca, gtgcaagttggggtgc,
ttaaaatcttagtacg, cggagcgtctgcggaa, gccgacttgatcgtat, cggggcccatagaatg,
gatattgagccaggga, ttcatacctcaccctt, ccacaagtatgtccaa, aggaccattagcttca,
ggccttgctgcatacc, aactcactaatactga, ttatccatatacacca, acggacccaggctctt,
aggacccacatgacgg, cggagatctcgccaca, cctcggagctccgcgc, gcttaagcagggctag,
aaatctgactctctta, gcatcacgaacagtat, atctctttatacaagg, tgttgtctcacctctt,
gggaccgggcgctagg, agcctttttttagaac, ggtccacccgccccta, gagttacggaaattaa,
ggcgcagggcttgcaa, tgacctactcctacaa, ctttatgaggatagac, cttacctcgtgatctt,
ggctctttgtgagggt, tccgcgctccgcgccc, tccatgaagttggaga, ataatgtatgcccgtg,
caatcataaatgtgat, aaaacctgtgtatcac, tgctatgacttaagag, aacgtagactacaatt,
tggaactacttaatgg, gtgggaccatagattt, ggtgtaaatcgtgtgc, atagagcgggcgccag,
ggggttcatatcaaag, cccacgaaaaaaaccc, ttacagattaatatac, gctcgtaagtcattga,
agacggctacggatga, gaggcattagaatcgt, agctggattaccttac, aggtgagacgctcaag,
atccctttaagttgac, ctaattgtcatgggag, ctcagcttaagcagga, cacctagctgccatgg,
ctcccccccccagcca, catggctggtgtgccg, ttgtgagttacaccct, acctttgcattgcagt,
attaatggatatatcc, tgtcctttttttacgt, atggttccccagccga, acaggaacagcagcgg,
gacagatttccggtgc, gcttaaagccagcacc, cacattcccgaattca, aaaagaggctaccaaa,
cttgttggatagttat, agatgctcgtgatagt, tgaatataggatgcct, gatgtataccgcacag,
ggcttgcactgtaatc, acacgttcaaatgata, tggctagtttcatggg, ccaggaaggtctgaaa,
ctcaggggccgcgcgc, ttttgggcaatcccct, caccgaaatactgtga, ctcatgtgtttactcc,
cctcgtcgccaagtca, caggggggcatttatc, ccgcccaactttgcct, tctcctegagtttaaa,
acgctcccatctccca, ggtagtctatactgga, cggcggagctaggctc, gtacaccgcatttttc,
tatcactaggacataa, ccccctgaggtgccgg, ccgcccctacattgca, cacttcgtcagggttg,
cactgtgctcgccggc, tgttgcacttagtgca, ccattgcccggatcag, gaatcccacaagogcc,
agaactcacagacatc, aatgatgcatatcatg, acttattgatcatcca, atggcattcggccagt,
caacggtgaaacaccg, ttaaccttatccaata, cttgcctggctctagg, gctgtatgtctcttgc,
ttcatggcctcggtgc, ccatgttagccatatg, aagtgtttggatagta, gactcagtccagagcc,
cagtgcaccggttttc, gcgtgagatttaataa, agcgctgggttcttga, ctcctgcacccacggc,
cagagtccggccacgt, ggaatgtgccaacacg, gttgttcacgtgtcaa, gacatacatagtggaa,
cgtgcacgttactagt, tgtgtatgtcatagca, attagttgctgtaatg, gtccgatctgccctcg,
tcaacaacttatgcac, aaaaaccgaagaaagg, gctgttgtgttgccca, ggcttcacctgatccc,
tcaatttatactgaga, ctccaattctaagttg, tggttttttcagcggc, acagtcattgcggctg,
aataagcctgagatag, ccatagctgatgatct, cggggggggaaaagga, agtttaaagcgttaga,
tcatatcctgtctaag, accattgagcactatc, cccctataggcaaact, tgttttcggctgggct,
taacagtgtgcctctc, ttggaagcagctggtc, ataggggttattccca, gtactcactggcttac,
cttattccttcgtcct, tgcctattttgagtgg, aattagattaggttct, gctaaagaacatgcta,
cactataaccagtaaa, aaacagttaatcttgt, aaaatccccttaagga, gatgaaacggattcag,
ggcctggaatgtattg, gtaaaatttaacgaac, gacggggtctgaccat, ggatcgttaaaatgta,
ctaggttaagaaggcc, gaagagtataggcgtt, gttttagttgtgataa, ggacgcccccccccat,
gacttgagtgagccaa, acttatactccctcaa, tatgaggagtgaggaa, gacgcctttgcggggg,
cagtggccctccaagg, ttatagctcagcatcc, gactccgtgtctgaca, cggagcagcctgagtg,
accgcttatttctgca, tcccccgtacacccag, gaaatacatgaacctc, ggattctttatccagt,
ctgaattgtgccatag, catttacatgccccaa, acacctgccgaccttc, agattggggggggaat,
caccgagtttgaaaca, tctcatcccacactaa, caccacttatgctgac, gctccaatagggggag,
ttaaggctgtcctgct, cgtggtgtatactctt, agagcattactgagtg, ccgccttactattttt,
acataagctcggtcac, gcaaaaaaatgcctac, aaaggcaatatctcag, tttatgtatgctagtt,
caaaggagacgaaaac, tcctgagagttctaca, tgaggcccacctcctt, ccataggcttctgcac,
gtcttacttaagcaca, ttatccaatccccaca, ggagactcgtcttgcg, ctgtgtcccccccatc,
gcccaagaccttgttg, ttatatcgacaatctt, ccagtaacatatatga, gcttgaatcctgaaat,
cgacaaaaaaacactc, ctaggcgtttttttta, ctcgcaaagtgtcagg, ctatctctctccatac,
ggagcaacgacaaaaa, aggcgtggcggccatg, agccaacccccccctc, ccccagccagacggtc,
atataacactcaaatg, ttgtagtttacctagt, attatgtgggccccca, ttgcaagtagagttta,
tgacgtctagaagctg, cttcaccttaacagca, ctagcatataactgca, gacacagcttttttta,
agagttaccgtaagct, atctggagcacctgaa, gatgtgcttacattga, atctgtgtgtataagg,
cagtctggctctagct, taaccatcttggtcag, tttctcctcgctcttc, atccccccccaggcca,
tggttcaatatactcg, tcaaccttataccact, tcagaccccgccccta, gcccacaggcaacgta,
ttagcttacattccat, ccctattaatcgtcca, agagcgagctggaaga, acgcggggtggacttc,
gtcaatgtattcttgt, gcttttacttacaacg, gattgcccccccagcc, tgtaggactccgttta,
ttattgttcttggaag, acaacacttcatgctc, taagtccccattacaa, gtcatcatgcccatca,
ccagcgcctacttaca, gtggggggaacgcttt, ctcctatggtatcaga, gactgttttttatcaa,
taaccttatccaatag, cgtgatccacacacat, atgctggcaaattagc, tccctggggggattat,
cccggggggaggggta, gttgaaaaaaaacatg, agcgaggcgctcgagt, tgttttttttggccgt,
gcattgcgccggcgca, ggtggggggctcaagt, ggggggggggtttttc, ctgcggtgtcattccc,
aaatcgcagcctcaag, gaggctacggggaccg, cgggcggggttgtgga, ccatatagtggttaat,
tatatgcgtaccattt, gcacccttttttttga, gtccccttgcttctcc, tcaaaccaccctaata,
cgtgtggaacgtccgt, cgcagttagggatccc, tagatgttatgctgaa, ccatgggctgcaaact,
ccacaagcgcccattt, cgacatttttttatgg, ttaaagttaactagag, gtgtaaatcgtgtgct,
gagtatgttgaaggcg, tcacttagtaaaatcc, aagattgagcatagag, ggttgtgttcaaaaat,
acagtccacggcttaa, gctgacgctgctccat, cggcgagaccacactt, aagcatgggcattgcc,
gtttacgaaaatgact, atcagctatgaagtga, gagcctgagaccttta, attagtcctgtacaat,
cgccccccccgaaaaa, tggatggtgctcttag, cgccccccccactcag, cacccccttattagga,
gttaaaccccgtctta, ctgtgagagttaccgt, gatggcggggggggtc, ctccacggagagcatg,
atgtctgcatgctcct, ctccccactcagtact, cagtgcacactcctgt, cgttgtcccttagaga,
ccatagtgaccccgca, gcgacacagttagacc, tgcaatttccttagag, actggatataaaaaac,
gtatgaagggagggac, ctaaaacctgtccttc, tcatcctttctataga, taccgaaaatttaaag,
cactttagctgtatct, aaaggtgtgggggggg, cccgttttgagtctga, ttgaagcagttaatca,
aaaccgaagaaaggag, acttactgaggcctgg, gcctgtagcggacagt, agcgcccgccttagct,
ccacctgcttatcttt, aggaggacacggtggc, gaatcttatgagtttt, gttctggaaggctccc,
ttgtgctaagatcgtg, gggtatctatcccgta, tggccataatatggcc, catctgcccccccaaa,
gcaacttatagctagg, gctgagagactatgtg, acgtggtgcgtggccc, cttgtgggctttccat,
gggggggggtaccctc, ttgggattaaactttg, gaagactgattatgat, gctgtactgatgctgc,
atctgaccctgatgcc, tgctgcgtttttttta, tcgaacaacagtgaaa, ccatcgaggtctagtt,
gcagtagagaagtcag, tcaagttgtccagact, cagccacacgagaaga, ctttttcgtatgttgc,
ttaggggggtagacat, caccgccctctaccta, accagcgttctagaag, tcttaagccacggccc,
ccttagctcaagcgct, cttcgccaagtgttct, gccagggcttagagta, agggtttgaccctgaa,
gtccacggcttaaagc, accgtctctggtcccc, gtggaagcgagccact, acaattctcagtatcc,
gggtactgaaatgatg, ccacatacccctgacc, ataatatggtgaaagc, gctttacagccctcga,
acttccgaattaatat, gatgactgttacttat, gataaagaggggacac, gaaaattcctcgcttt,
gagcttggagcatacg, tcttcagtttcgtctt, tatcccctattaatcg, tacagcatcacgaaca,
cttctcagccgtggct, ttttatttctacggtg, cccttcccccacacta, tcctattgcctgcgca,
ggaaaaaaaacgggta, agttactcataaagag, ctacgctaaaaaaaat, gtgagcaacctccttc,
ctctatctcagtcagc, ctggtgtgccgagcct, gccctcggttggtttg, ttaggggggggggtta,
taggttaagaaggcca, ggcccaagtacacccc, gcgatctgaggatgct, aagtccccccccagga,
accaattagacttaga, ggaacatgagcccccg, aagtcctacctttctc, ctgggggacacccgca,
tccgcctccgcaggag, cggtggggggggggta, cggccagccgtgcctt, ctcttgttgttgcaat,
ggatactcaggcatgg, gacatgaacctttttg, gttagtcaatgcctaa, gtacggagtaaactac,
tcttattacatgcgtg, ggagttgcgatggtgt, tggtactctgctgttc, tcagcactttttttag,
gttaagctagaattat, gtgaccgtttggacag, gacaccgatgatgcag, ctatctctacaccctc,
ctccaaggcattagta, tgagggggggggggag, ctcctggtgacccgag, agacggcattttgacc,
acattgttatcccaac, tcgtgcacagtctaag, ataacgccccacatgt, gtctgcatgcgtgttt,
cgtgggaagtgcccct, aacttgccattctaag, ctaatggcactaattg, tagccccccccaaggg,
ttcttgcaatagtagg, actaaaagacctattt, ctcttttgcaggagac, tatgttgatctaggtg,
gcgcgcgcgcggctgg, cccccgaggtacctcg, cttagcgcagctgcac, tcgtgggctgctccgt,
caggtatgaagtacct, aaggcttgaatgcacc, aacttgtttacgttta, ccttgttttttttgat,
ggaggataggcagtag, tcaaaagaatatacgt, accatatgggtgacaa, gagtgacctcgggggg,
atgtcattgcgggtga, tccatgggcaacgcaa, cccagcgttatgcctg, ataactaagtgtacct,
cctgttccttaaactg, agagcttggcccggag, cgcacccatcctaact, cttaggtaccgggttt,
tgtatcatttacgcct, atttcatctctaaacc, cccaatgaaaagcccg, gttagggctagtctct,
tcgcaagctggctcac, ttcctgcagatagatg, gtgagacctccaaagg, agcatatagcaccacc,
tacaattatggtggac, atggcgtggactctgg, ccttgtgttgtaggca, gataggtagcctgagt,
ttggattctgccttgt, agtcgtggtgtctctg, ataacgagaactggac, gtaacacttatcgtga,
gactcagcttagagct, ggcggtccgcgcgaga, ttagtttgcactagag, gtaacacccgaaagag,
ccagatgtcagttggc, cggggtttttttagga, gctgcccatattgttc, gcttccccccccagca,
ttctgtgctctgaccg, gaatgtccccccccca, acctcccattccgcag, cgttttttttgtataa,
taccggtaattaattt, tgcctactataatcca, aagttatgcccttctt, gcagttttttttggca,
gataggtatatcgaaa, atgtaagcctgtcttc, gctatttccttttacg, ctggacacccaccgat,
caagacctcgctgggc, cctgatattatactcc, taacctggttatgacc, ccaatgcggccggcct,
gttgtccacaactgta, attaacaagtgaaggc, cctttggttactggga, gtgtcgcatgcctaca,
tgctatgcgtagatga, gggattgtttccaagc, agtccaaggcccctgg, agttcaactcattaag,
cgactgtataaggcat, cgcaaggtgctctctc, ataagctatgagcctg, cggtttgtactgatgt,
gtcgggggccgtggag, ccggataaggaagttc, atttcgggtgtttttt, cactggccaatttggc,
gccgttttttttacat, cttatagataattaag, gcttcgagaatttccc, cacactagcacttcac,
ggtctcttagtggtgt, gacactctatcttgca, tgtattaggagcctga, ggctaaggggaagttt,
gtgttaattcctgaaa, acagtatctgcattgt, tgaggggggggggacc, acaatgtagctcctat,
ttagcctaagtatctc, gaggcctcaatgggga, aataatttgtaggcct, ctccgcatggtgcgag,
gcttgtgctggctcgc, cagaagtttaattaac, tcttctattctagccc, ccccccccacaggagg,
tatgcatgtttataga, acattttcctcgaagt, actactacctgctgat, ttggcacgaaaaaaag,
attgagtttcatggtg, actgtgtgaagcgtct, taccagtccagcctgg, ggacctcttagctttg,
cgcgttttcaccggaa, acagaaaaaacggacc, tgggccccccaactgc, gttgtgtttttgacgg,
tctatatgcaccatta, aatatagtgatggacc, gcttttttttacacgt, tactccccccacttgt,
gttgactgcaccttgg, tttgatttcgtatact, gtatgtccaggtgaca, gtttccaacgcccagg,
gtgtccctgagacatc, cgaagatgattcttga, gtagccattgtggatt, attgatgagtgcctta,
cttctttttcacgact, atacgccttactgttc, cccactggtagaagac, tactgctccctaccac,
ccgagccaagtatggg, tcttatgggggggtgt, cctgcaagtggcttga, gagaactatgctggtg,
acctcaccataataag, tggaggtcatgacgtt, gtacaatggctcgacc, tgatccatgctgaaaa,
cggggcggggggtagt, cctgcatcaccctgtc, tgcagacgaaaaaaat, aacactgatgtaacga,
gtagagaagtatacac, gtaccgcacctggcct, tgcacctgctcgtaaa, gaatgatggattgtct,
actgttcctcgaaaac, ccaatagggggaggct, tcagccatactggcta, cttaatggtatctcga,
gggatcatatgattaa, agtccctctctctgcg, cacgagttagactgtg, cttaatatctacaata,
gtgggcatagctgtag, ccatacccggggccac, tggcttaggataggat, caagaccagctcatat,
ggacaccccttagcct, ctgtcgacaccccagg, acgcagtgtgcccagc, gtgccacctttattgg,
cttatgagggcctatg, atctgtattcgattga, ttcccccccttagtat, cataaaatcgcagcct,
tttgcaccgcttgtga, acgatggatgaggcag, agctcatgcacgctgg, aggtccacccgcccct,
atgacttgcgaggcct, ctgtttatacaggtgg, tccctagcagtgtaat, ttgacttatcagactc,
gtaaacgtatcagttg, aggtttttttatcact, gctgaggtggaggtac, ctgaccctgatgcccg,
tccttagagcacacag, gttacagagcttacac, tttctttttgtacgta, caggcggataacctta,
gctcggttaatacggc, atgggatgagcttagg, agatcccggcatcctt, tctagtgcgatcacct,
gacctgcttcgctgct, aggtacaaggttcaac, tgaggctgcgaggctt, gacaagctgatatagg,
gcacagcattgctatc, tgagaatgcacaggcc, cacaaatatcatggac, atttgaggggacaaca,
catagcagcgattgta, agttaattatgactct, tacgaggtatttagag, agccctccccccccaa,
tcttactgtttgcaag, agccacgcccacctga, tttttaggggggggct, tatcagttccaccccg,
tcagaacactgtatta, ttccatcgttcctccc, tcttggcgaggcgcgg, ccttaaatggcatgaa,
aaatacctaactgagc, acttacactggtatca, cctctgaagcccccta, taaagaagatgattgg,
atcacacgataaagtc, gtctgtgtcacgcggc, cggcactaattgtttt, ctcggaaatcataagt,
atccagaagaacaatt, ttacttcatcaattag, cgtcctgttttggttg, atgcttcttgttaagt,
tgtgttaactgcaact, tcattatgtttgtaac, aaatgtccagaaggac, atcgtcaaatatcccc,
gctcctccacataggg, tctgtggcagagagcg, ctccccttatcgcagg, gcccctaaaccccata,
gtttagttacccacaa, accatctgaacaggca, tcgaacagtatataca, ggagattgttttttag,
ggaaatgagcgtagtg, aagcaagatgcccaat, gggtctgatcaagtcc, gcctcctgtgcctcgg,
ggtgaagacggatgcg, catcctaggtgaagca, ctagctgattgaagtt, gaagggaaagagcgag,
ccatgcgtactattcc, tcttgtgtggggggta, tctaaaaaaaaggtgc, gtgggaccttcgggca,
atcaaattagttagaa, tgacatcctaaggcat, cagtcagatttaggct, aaaacgggggggttgt,
tccacaccaagaaggt, gatccttttttttatg, gtaattaagattcatc, ggaccgggcgctaggc,
gttagcgaattaataa, aagtggcgcaaggata, tatcactcccccctac, ggacccttccccaata,
gagttaaacaccattg, cggaaatgtgccttgg, aatatcatcagacctt, aaccatttttccccgt,
tctagcttgtataagg, tcatttacagttgctt, actgtcttaaatgtag, cttgcccccccagctt,
attaccoggataatag, aaaatctaagctccgt, agagccgcctgcccct, gagtcccaatgagaac,
ctctcacaatcaaggt, cttggcagggctaaat, ctcacagctcaagcaa, atctacagtcatgtcg,
aagcgtgccagagggc, ggataaagtctaggta, tgggcctggacaacgc, ccgtgaccaagagcag,
ttgaatctgctggctc, tgacatccatcctcag, catgatcatcacactg, tgttccccccatcaac,
cccgaccccaaatata, tcctagaccgggtggt, tgaacacctaggtaat, agcaaaaacgatccaa,
ttaacagaagtatgca, ccccaatggcagtggc, aagtgaaatatcgtca, gatgttttttttgcaa,
gactccagttgtacac, cgatgaaaggagcatg, acgaggtcctgcttcc, accgcaggggggggcc,
ctaaaaaaagaggcga, ttaaatacggatataa, catttttgtcgattga, tttagggggggggagg,
tcctaaaatggtgcca, acccatcttaaagtaa, tagtttttgtagaatg, ttagatagtgtctaga,
gcacgtgccacgatgg, gcagatcaggaccact, gataatgtgtaagagt, agcaaagctactggtc,
cgatactccatcccag, cggggttcgtcttctc, agaactagactccctc, ttccttttatgtcacc,
gagccctaggcgggct, ctatttgcttgtccct, gatggttaaaccacat, caccacttaactggta,
tttcccagacgccttt, aatcgaaaccgtctga, actctttaccatttaa, atattgaaggactcca,
ttagtattccccccaa, ttgagggcgtagaagg, tttatatgattttcgg, aggctgactatgaagc,
ttcgggggttaccgcg, cgggggggggcaccag, aactcaacccattgtg, cttgcgaccaacctgg,
ggtattttttttcgga, acagagttagaccccc, tgtccataacttacct, agggccagttacgggg,
tacccagggataaatc, tcagttcaaccataga, atcttaaaaaaaacgg, cttagtcagaagtata,
tacactgcaaatgttg, ggcgacaggagttaaa, gggccccaaggcttta, gttgaagtttatgagt,
ttgtgccccgtctcct, cagcgcccgccttagc, tgaaaccctggacttc, caccaaacgtctgcct,
agggggcttaggcacg, acgcagatgaggtacg, acatgttacctagaac, atctattcgtgcacag,
gatccttctggttggt, agagactgggtaaccc, tatggggggggcatgt, ccccctatctgggaag,
gatggatggagtaaga, cgttgttgaaattttc, agtcaagggcctcctt, tgtatatcaggctgtc,
aatgcctttcaggagc, agtgccgtcctcttca, cgccttagcctccgcc, tcaacagctaggcggg,
cccgtgaccaagagca, cctcattgccattaca, tctcactttactgagg, ccaccattgtgctctt,
tgcacgagcttaagca, gctctgtgggtttatg, aagctcccacttccct, cctcacgttgtccagg,
ttcgactccatgtctt, tgccgagttattaggg, aagcaaatgtatgggt, gagagcactgcttaac,
cactcaccgcatgacg, ggcaggtgccggaaaa, gggggaaatgtgcgcg, tgaataacaggcatat,
caagacctctggtctt, tgtttatcccaagaac, aattctaaaaaacacg, tttccttaatacctcc,
aaatatcagcttgtgg, ctgccgacccctccaa, tgttaaaatccccccc, ccatagaatggagctg,
ggtggaggcctaaagt, gtagtcggcgtgccag, tctgttccatttccgg, ttcaagatgggggggc,
agcagcttccgttgca, ctacatatgccactta, ttcgtcttatcgaatt, agtgttaccactatag,
caggacccacatgacg, taactcaccaataaca, tttctatctcccccga, tttggccttgtccgag,
cgcaagaaggattaga, ctgggcgccagagtta, ccagtcagcaagatct, catgattgaactacct,
tacttaggagaaactg, acccaacatgctaaac, actgtgtcccccccat, tacatagctgcagtct,
cggaggcccttccagg, tggtggccatgtaggt, gtccgtcagcgtgtgg, ggggggggtatattct,
gcagcattgataactg, ttgccatccccccctg, tccagattagagttag, gttaacctttgaatca,
tccagcaccattggct, gattagtcatcttgtg, gccaggggccggactc, tcgaaatatttaccca,
tcccagtcgtggtgtc, tggcctaactagaatt, ggaagtcatacagaga, gtccctaaatgttttt,
caagcacctgatgtgg, tcccattaggctacca, atatccacattgttac, ggatcattttttttgg,
ctatgacaaacactaa, gtctgaacctttcaag, tcttagagctaaaaac, taaatttagttatgta,
cattttttttgaccta, accacttctggttgag, gcatgagcaacgccga, gttcccccgaaaaaac,
cttacaaatatagccc, tttaaccatcttggtc, ttctttagattaagag, aagaacgagtataaaa,
catgtggaccagtttg, aatataaatgcgaatg, agatgctcccctctgc, caatcatctgtgaggg,
gcctcccgctatgcag, tccaaaatgagtactc, aaggctggtctgaaat, aaattagccgtaaatc,
tggcagctccaagagc, gacattgtgtcttggc, agttactgaatatgca, ctgtattatctcctac,
ttttcaatccataggg, gtgttatgccgtagca, aacaggatagttggaa, tatgattaaatcgcca,
ctaatgtgaccagatg, gccgcccatgcagtaa, agaagttatgctgtac, ttgtctgacaacaacc,
tggcttggtagttatg, tctggtatgctgaggc, acagaggtaagtgcag, ctagatgtggctctta,
tcgggcgctctgatct, caagtacgatgtcttt, gactgagaggcaatat, tatctggtgtttaact,
ctacaggatctcgaaa, accaacgtgtagaaac, ttccagggtcttatgc, tacacaaaaggtgggc,
tcgctgttatttccaa, cttttaggtatcttgt, atgtgttacccgcaca, actctgataactctta,
agaatccaatggtgga, ccatctgtgagacccc, atgcgtgcagtctcta, gcttttttttaacagt,
tcatatactgttggac, ttaagcctcgctgatc, catatgaactactact, ctctatgagtagtcat,
ctcacttttggtgtta, aatgcaggcccccttt, acattttggtagccaa, cttgtgacttggtgtg,
cggatgcgggttctgg, cgtatttagcaatctt, actaatttgacagtcc, tttacggctttatagt,
tgtcactgtgctcgcc, ggtgtctacataacct, acattagatttgcaga, aatgccccccccccat,
ggtgacagagtaccac, tagcgacttttttccc, taaaagcgttccttaa, tacgcccatatggcct,
aacaatgctattgcct, cgatgagactctgaga, gagtatcgtttgaggt, tatggtccaagccttt,
ctttggccttgtccga, gtgctttgtttatgga, tctgactttcaatacc, tacataactggcctaa,
aacactgccgagttat, ttctcgttaaaaaatt, caggagacgggcaatt, gggacgtcgaaggcag,
cacatgggatgaagat, gctttggtcagcgact, ctttcttaccgttaat, cactcttcttaacccc,
ctttggcacagatggc, gagtttgagtgagccg, ccagcttctgggcgtg, tggatatggggggggc,
atgagaccacccttct, gtgttatccaataggt, cgtggaaatgagtcca, gacgttaaacagcaca,
aggaatattagagtgt, cagccccaggtaaagg, ctcataccctgcaaga, cgtagaacaaaaaaat,
ggacctaccacatacg, atgtggaaggacctta, ggctccaacattattc, ttagctgtccccccct,
actctcccccgggagc, tggagtccatcagctt, ttttacataggcctac, tcattggaggacatat,
actctgtacaatgtgg, cccacttccccggtgg, tccccaatggcaagtg, tccagggttaccacct,
acgatgagattatatc, ccttttaagtttagca, gtagctccaagcaact, aatagtcaatatgcct,
ttatattttgatgacg, atgggagggcaaatac, acttgtaaagccaatt, tgctggcacacggaag,
attaagagttatctgt, cgcctttgtgtgaaaa, ggcgggagcacccctg, cgagaaaaatggagat,
agttgagaatcacacc, ggaaatcttataaggt, attgtgagaccataat, cgctgggcgcacgctc,
tatactgttcgggtta, attattatggtctgtt, cgagagagaaagaata, tacgtattaatgaggc,
tggtatataagtctta, cagtctaaaaaatggc, atgagccgaaatccca, ccgcctccgcaggaga,
aagccagtgtcgccgc, tgtcgacagccttaat, gtgtccggaacacagg, atctaggtgggtgcct,
ctttatgatagatcaa, tatgatagctatatta, ctatagtccacgagaa, ttatacccccccccga,
ccctaaaaaaagttct, ccaacccagtcataat, cgcttttaaactatct, ttatcaacctttaagc,
gcagttgtctgccaac, gtgcaataaaaaaacg, gacaaatccccagcca, ggttggcaaaaaaacg,
gatacttcacttttca, caataccaagcgaggc, ttggggtgtgactcaa, cccggatagggggagg,
actgtgttatcgagga, tattccagtcaacccc, tgagcctccccccctg, ttggatactcccccca,
actcggaatagatgtg, tatttttctgtcggtg, tacataacctggatct, ctggtgtccccctttt,
aacaagactaataggt, ttacctcattactagg, tcttgcgctaatctgc, agtcaatcataaaacc,
cgggttaggtgactca, cagcttttcccccccc, actttctcactaataa, tgatcagatagagtgc,
ggaggcgccggtgtcc, tcgcctctgaagcccc, acgcatgtgagttacc, gatttaaatggctagc,
atctaggtatataagg, gagtctagcggcacgg, aaagctcaccttgtgg, gcaaggcactggtact,
aagaccatacataaac, atcaaaaggttgcatg, gtttagggcccttttc, cgcggtgtcgcacgcc,
gcgggcgggggagaat, caagttggcaccatag, cccctcaaatgcatta, tagtgaccccgcaagg,
aattccatccccgaaa, ggcgctgggctctcgc, attcgaatttgagaca, ctccgcaggagagcag,
agtgagattagcagta, caagaggaggcctttg, ttgaccaggtgccgtg, tattctggtctatcca,
taacgaatgcattata, ttccatctcagcggct, gcgcgggctaagccct, ggatagggggaggctc,
ttccgtgactctctcg, caacatgtctaatgat, cccggggttcgtcttc, gcctggaagacataat,
tgtaacaaagttacgc, agttacctcttgaaaa, taatcaagaattctaa, cattttttgtcgatga,
ctgtgtattggacctt, gcttagagggagcgtc, aaaactcgtgttattt, gccggcgcttttcagt,
ggacttaacccctact, ttgtttagcagcataa, actccttacttggacc, atattctcagacccaa,
atgactaggtgaagga, aacactctttcctatt, ggcttgagaggatgca, ccagaaggctgatgca,
gtttaggcaactggca, tgcgtctgccccacag, gagggggcaaaaagat, gacggcatcctctgtg,
ggcttactaggtgtga, gcccctgaccaagtta, gaaagtaaatattcga, tcttcctttgtgcgcc,
aatctggttaaaccct, ttgcccggatcaggaa, gtgttattactgtaag, taaaagacgatattaa,
ccgcttatttctgcac, ggtgttttttttacac, cacaatgtatgcccta, acccagcgaaaaatca,
agaaatgtcattgcgg, tttctctaacagtctg, gggcttaggcctaaaa, ttaatgctttttttcg,
cattgtgtaaagcagc, tgcaggacctctgagt, agctctgattgacgaa, ccagcgggcgctgcgc,
cacgaagagtgataat, atctagtcctggtgca, ctttggtgtgaacaac, agctgacagtctattg,
aagaaactttaggtgc, aatttttgatttgtcg, gacacaaagtgtgggg, acaagtgaaatatcgt,
cctgtgggagtgtaat, catggttatactctta, tctgatgccgagccta, attgaccatgttctgg,
aggattacccatggtg, gattttccttatgtgg, gggaggatataggatc, ttagggcgggggtgag,
tccagacccggatagg, tcagtgggtagaggtg, tgccggggggggttgt, tgcacccttttttttg,
gttaagtggtttatag, gaacttaaagcctagg, accctttttttgggct, aatatgcatgttagcg,
gctttgcttcactatg, ctcgtttctgagcctc, ttcagtcttaccctaa, gcactttcaaaagcta,
gtcaagctggagtgtg, tcgtgagagcagcaga, gggaaagccatcttct, atttcgattgaagaaa,
tttaggatcgaaaaca, gtatgtctacctactg, ggactctgcgatgcat, tggtgcccctctatgc,
atacccaagaagggat, agtcttatgtagtcag, cttaagggggggagta, tagaaaccgtgcagtg,
acatagtgagggccac, tccgtcttacacattc, attgtatctcagttgg, taccgaaaaaattatt,
attcttccaccggcca, gggacctgcgtgtgcc, aacatggattcactac, atcaaacgtgacatta,
actccaatcccgttca, atgggggggatccctg, tttccttaggcttacc, acattagcctaatggc,
ttcagctcatgggctg, cttgtccaaacttatt, cccaacgaggctactg, ctgatctaaattacaa,
atatgaactggtgcag, gggatttaaagttctg, actattttgggctttc, gtcggggggcgcatat,
taacttgatgttggga, gattatcccggggggg, aattagcttttaccag, ttagatacgccccatt,
ggtcggggatgggcct, gcacgagcttaagcag, gcacttgccgcagtca, aaccctgtgtgaaatt,
gacccaatcctacaat, ggctgaagctattgca, gagccgcggtccccgt, cctttaaccaacagct,
caacctagcattgaat, ttttcgaaattaggga, ccatccacaatccatt, tgaaacttttgacctc,
gtcggtccgcatgcag, tggcgtgcacgttact, cagatgcctactctcg, cacctcgagggtcctc,
gtccgcatgcagctga, ctacttgtttcttagg, aaaatatgacgcaaat, ttctactggctcatcc,
tagcccttagtggtga, aagctgagcgccatcg, aaaacctcctggttgg, tttacaaaagtaccct,
cttgaataggtcttca, cacctcgcteggcccg, agtaattagcttctgt, atttgtcagcgtgtgg,
cgttgcggcgaggggc, agatccaggcaaagtt, ggggatggatgaattt, cacgaacagtatagct,
tccttaagagattacc, aggtaaagtctcaagc, taatggacttttagga, tgttaagcagtcttag,
ctagggattaaaccta, gatggctagacccata, catgaccagtgcccat, atgctttttttcggcc,
actcggtttttattta, aaaagcgttcagaatc, agtgctaggctgcaca, ctcctttcgcttcccg,
acagagggatgttcca, tcctagggaaggggat, tcgcaactaatttgtg, tccatttccggtaaac,
gttgataaaggttgtt, cgtagggcaggagtac, acttctcatagagtat, tagcgccgggcgcatt,
gtttaagtttctccaa, aatgactcaacagaga, gataagcccgggcaac, gggggggctgactatg,
ccttgctgtgattcct, ctttaaagagggatgc, gcagcccccccccaaa, cgcatgcagctgacgc,
gtcccagtttatggta, agggacttgcctcgta, ttctagagggttgatg, acgccggcggctgaga,
gggaaaaaaaaccgct, ggcacacttactcctt, cttatgagtgagtcac, ctctgtgtggacaaag,
gagcgtcttcttacta, gatctacataatgcct, accattatattccagg, tctgtgcaactgtttc,
gtggccgtgctttccg, ctatgttctgaagaac, cgaccctaaggctgcg, cctctactctctttat,
cacaccctcgcaagtg, cttgatccgcccaact, tctccacattagctgt, ggcatcccgggtttga,
agctattctcgcacca, tgaaccttagaggtat, tcaagacttgaaggta, tcttagggctgcagac,
taggggggggggctga, atctagaaatggttat, cagtcttactactcag, gaggatggggggggga,
atgccacgaagtccaa, ttcctcctatgagtta, gttccccatgagagtg, gcctcgaacacctgct,
tcattttccgaactca, aggcacggcgccacgc, atcagaggcacgtccc, gatgcagtcttggtcc,
gcaaataggccccctt, accgtagtccattaga, acataagtttggtgaa, cggcgcgggctaagcc,
aacaggatcagtccat, aacagttccagtgatg, gttagaaactcacaag, cctccttctatactta,
cagatggcgttgttgt, agcagcccccccccaa, attgagtgaagccccg, gttcgattgaaaccca,
agggaacataattgaa, gggactatcattaaca, agcgtagactttgaat, gttatggagctgttcc,
gcgcacccgccccccg, agccgtaaatatgctt, caaaaatttcgagtct, tgcattgccggatgag,
tgtaatatggttaggt, aggcaacctattaaag, tctaatcggctactaa, cccacccccccatgaa,
aggggatagagccgtg, agttagaccgtgtctt, gcatgggggggatccc, tgtggccaaaaaaaac,
aatttgaggggacaac, acctgactattactta, ggtcccagattgatac, gaataagacggcattt,
aaagtagcgcgaggcc, ttccaacgtgcttaga, aagagcttgcggacaa, tgcccccccccgacta,
gagtgatgctaggctt, gaagagtaaccttaaa, gttaagagtgagtctg, cttcgcattgaccttt,
gaaaatagggtacagg, agagtaaagagtgaca, actaaactgagtgtaa, ggtcacaggggctact,
cgccccccgtcaatca, tattacccacgatgaa, atgggtggagtgagtc, aaatacaagcggattc,
catcgcctgctgtgaa, gggagtcttaggtagg, gaggtgggcgtatcag, gttatgggatcagaag,
tctaagaggggagctt, acatggcttaacctcg, gcctaggataatctgt, tgcggactgcagtgtc,
ttcagcaagtacgatg, gacaggatcagcacgg, gtccccagattgtctg, ttgctcccccccggcc,
attgagcagtctcagg, tcttaaccaaatagta, gtggtctcttcgtegg, taaggcaccccccacc,
caacgagatgattttt, gctaggctgcacagtt, gttagatgttacgtta, aaccctagcttcctct,
ggacaagagtcacgca, tctttgtgctccgtgg, atatacttcatcaagc, aaagtagtgggtccct,
agtgcaccggttttca, ttgcatcacagcagca, ctgtagtcattagaat, tgagtccatgtgctcc,
ccatgagcattcagat, gtgttagtgacgtttt, cttagggtgggggggt, atacgctctaagaatg,
gccatttgttcctagc, caagaatcaatgaggg, agggtttcaatttcag, ctcggcaaaggcctct,
cagaactatacaggag, taactatcccccccaa, ccgattttttttggca, atagcagagcgagaaa,
gaacagctcatagggg, gatccgctgtacgact, atttttaagcagcgtc, ccaactcctacttagc,
aactggtgcgttatga, attatccgcatatttc, ctaggctggagaggat, acatctactgttcctc,
tctgtcagatggccgg, tttagaagacctccta, tagccttttttaagca, tgccttcccgcttagg,
cccgccacaagtatgt, gattgattttcttgtc, cccctcatgtacccat, ttggcctaaagctatt,
ctagagcaaattctat, cgcctgacctataaag, accatttagtcagact, ggcgaaaaatcaatag,
tgccttccacccaggt, ttgccttcttccccga, caggtgtacggtacac, cgaggcattttctggg,
gagtgtagatgggctg, agtccgtctataaagc, ttttagagtgaaccag, tgttgaattgggttag,
gggcgggctctttgtc, aacgagcttcgctgtt, gccgagcctaagctgg, ggatctaaccctgatg,
ttttcgacatgttaaa, tgtcataacctaattg, atcttgggtttaaata, ttccgctcgcggcctt,
gcgtcagaagctgcat, tacaatgggatggctt, ttagcctcccgtgtaa, tgaccgctacatctcc,
atggttaaaccgtgtc, agcgagcaactgtaga, agttgggggggcggac, aactggatcccctttg,
atctttacagaccgta, gtaccaccaccaatcc, accatccgatttgtca, tagatcaaaaaaaagg,
tatgtggatggcagag, gggaggtgtagatagc, aacggctggggggaga, aagtgacgtgtaagat,
gttgcccaagtccagc, atgtgatggcggcctg, attaaattttagacga, cttactgtcgccctgg,
actgcttatcctgcat, gtaccctgttctctta, gaataggcaatttctt, caccgtaccctttttt,
ccccgatacgagtctc, gtatggtccccgccga, ggcctaaggcagcggg, aactgctgtgacagtc,
ttggctggggcgacct, caatcgatctttgtgt, gtagacgctggaccca, atgtttccgttaattt,
ctgactaggtgtgact, caccagagcaatgccg, acgcccatatggcctg, ctagtctatgaacaat,
cttagacgatgggatt, gctttaccccaatacg, tatctcgttgggtttg, ctttggcttgtgtggg,
ctgtgacgtgtgcatg, agatctcgttagaccc, tggttttttttaggcc, gatgcgagattgcgcc,
agagaccgagggcact, ttcgtacatttttatc, atgacctaaacgcttc, gccttgccgcgcccgc,
aaatgcacatttagtc, tccttacctcgagatc, aataatactctgtcgt, ctttatatgttggaga,
aatcaatcccagttgt, agctgtgcgtcctcca, atttttttctagcacg, ctgcgccccagaggcc,
acccatcacagggcta, tgctgataacaagtgg, gttgtcccttagagat, gggagactcctctcgg,
gtaatccaagattacg, tgtttattcgtaatta, gagtgtaagcttgagg, gcgttatcctgggagg,
ggggtattgcttcgct, cgagcttttttttaga, gagggtgatcatagag, ggggggggattgagct,
tgacggttcctggctg, ctgctattggagctcg, tcaaaaatttcgagtc, ggcccacgcctggctc,
gggacaaaggctttaa, ggggcatcccccccac, tgtcagatcagacctt, atggttatactcttaa,
ctgcccaactactcca, agaccaactaaaacta, gttgagaggtctaaaa, atcgaggtctagttca,
cgttttaattgggcgg, atagctcactaataag, gttgcctgattctgcg, tcacacctagagcaga,
agaccgactcatcttc, ggggcaggatgatgta, tgggcatgcggaatgg, gccgttggctccgtcc,
cctccatgttttgcca, aattaactaatgttgc, gcggccatgccaatgc, attccacttgttttgg,
cttaggcggcggggtc, ttgctgtcgacctgta, gagaaccccgaaacaa, ccccaaggaattccta,
ttcgaaaaaaaatacg, ggtgagttaatggctt, gttaaaaaagccatct, gggaagcccatatggc,
gtctattatgaactta, acaccacttaactggt, ataaactgctgctaga, cttgtaaatataggca,
tctggtcttttgatat, caattggcgtggaatt, aaacgtccagccttaa, gaatgtgcttaaatgg,
gcaatgaatatatcat, atcactggccgttttt, caactgagatatacct, cgtacatatagagaca,
acgttggtcttcccaa, aatgcacatttagtca, cccagttcaggacatt, taggagtgcaaaccag,
gtgccggtgtgaggca, tcccccacaaagacat, gctgtccgggtgaggc, cggccttgctttacgg,
cttagagtctcacacc, ccatgcagccttgact, tccagccctcgatttg, caggcttgccactttg,
ccctgacttacgcatt, catatctgatggatgc, cacctgataaacctta, aatgtcgtttttttgg,
cgtatttccactgagg, ctggcatccgtgtcag, ttgggagggtcatatt, cgttcagaatctctcc,
agactgcataaggatg, gcaaaaaattagcatc, aatatgctatagctga, gataatgatggccccg,
actgcctaggctgttc, gtactacaaaaaacac, catttaaacgaatatt, gagcggccagccgtcc,
gcccttttgtagctcc, tgggaggcgcaaggag, aaaacgaccgatgaaa, gtgagcatattgagtt,
ttattaaagctaactc, ggaggcccggtgggcg, tcagacctccctctgt, actggggggggtgtgc,
atgggggggaagacca, gttccggttcaggaca, gattaacgactgaatt, gcgtgtctgttttctg,
ggggaagcccatatgg, attttgcctagcagta, ttccgtctgttcattt, gtggaaccttataatg,
gcaacttcaacaaggg, actatcaagaccacag, gtggattttatggcaa, caatgcacctgctcgt,
ttagtcatttggtaag, gatgagtcagagtgcc, ttaccatcgaggtcta, ttaagaacctctaggt,
actgcaaaggtattgg, attgattaagttactt, ccattagggcaggcta, atgaggaaggtcaaac,
ctagagtttagcatga, tccctgtaaaattctg, gtgcactattatggat, aggatgatgtaaatca,
ccatgttttattatgc, gtgcctactctgatta, aagcatccatagagaa, ccatgaggcattgttt,
agccttactctgggtc, ttaggcaagaccaacc, agagcaaacggccaaa, atgtcctgtgcttagc,
ggcctgaaagtcctgg, gtttcatatcgttttt, gaatcaacgacttgtg, tgtctctgaaccactc,
gatgcattattatatg, ccgaactgaaactatg, ttttcattggagatgc, cttgttctcccgcaaa,
cctaaggggaagtgca, agctcttatctgttat, cctcttcttagggcca, catacaggcctaactc,
gggtgttcttacttgt, aaaccttttgcactac, atgctaaaaaaaggct, tccagtctaaggactg,
gacttttgagtgttag, cggcaaacttaggcag, ttgacggcacagtaag, aatgcggataccttag,
cttttgccggcttaga, tcttacgggctgaagg, ttaggggcctaggcca, tttgaccgcgttagcc,
aattaagccttgtgcg, tttgctatgggtggat, ggtaagtgtctgattt, ggaaaaaaacgcctac,
ggtgtcccgggacggg, ctgatgcctaattgtg, cggcctctcccaggac, atcccatagctttggt,
ctccggcagttgtaaa, ctggcttgcgccaact, ctctgatgtgtataac, taccctagttccggga,
aaccgccccggtctcc, cctggcatcttattgc, cttaatgaaggtttac, ccactcataagcatca,
atacctgttcaactaa, tgtaaggcgggtggct, cagtatcatgttattg, gtcgtgccacttcaca,
ttaagcacaagaataa, gatcattgtgttatag, ggcttggactatggtc, agtgttcttaatgacc,
aaaacggggggttctc, gtcctcaaaaaaagca, cctttagagacgacaa, gtgagtaaaaaaaacc,
tctggcttcctaatta, actaatgaacgtgaag, ttctctaacaccttta, cgaccccgccctgcac,
cagccatatcatttac, aaatcaggcagttgtg, gcttaggggggggggt, acagtcagcaatgccg,
tggagtcacttctcta, tacccagctcgatttt, ggatcagcgccccgtc, tagtgaagtacctctg,
tttcccggtccccaga, cccatccgttgcatgg, tgcgtaccattttttt, gcctggcaatgcggta,
ttaggtgtggatgcat, cttgcctaggttcctt, ctctgttaaagggtgt, ttgcccccccccgact,
caagtaagttactaag, tgcacaggatcttaag, gtgattgcaaaaaaac, gtatctcgaatggtaa,
caacctatacatatct, taagatgtgggctaac, cagagcttaaaaccag, ggtaatgcttgttttg,
ctgtctggcctgttct, aagctgattatgtgat, ctgagctcacactaat, tccatgacaagacatg,
cttagttgctccaatt, atacgaaacttatttc, tgtccccgagtctatt, ccaagtttaatgctat,
cgccacccacccctag, ttagagcagactaatg, tggtcagcgactcaac, gtgcaaatccaagtta,
ttaggcaggcatatca, aggagtagctaccacg, agtagcgggactacat, ttaatcgtccatggaa,
ccaggtccgtcactga, atgccgcatgggcttc, gttgacggcacagtaa, aggaataccataaagc,
gtattacaaggtctac, gtgatattggtattaa, cacggtgggcggcgca, atagcttcccccccca,
caggcgggctttcaga, ggcgcacaatacgccc, aaccatgatgtgaggc, aaatggtcccccccga,
ccaaaaaaagagtagc, gattagtgcagtggct, ttgtggtgttcgtctt, gggggggtctcctgtt,
gctctgctccactaag, cccactgtcttatcaa, ttagggtggattcaaa, tttcgaaatgcctatt,
gttgagggcgtagaag, gacataattcctgagt, gctgttaagcagcaat, taaacacattggatgg,
tattcgtgcacagtct, catcttaaacttggta, ttaactccgtctccca, tgcaagtttttttgcc,
cttatggtgctttagt, tcttaatgacacaggt, gcctgacttcccctga, gggcatgtcatgggct,
atgaataggctcagac, gactataaacagaact, gcgcagtctctgtata, cgccaggggggtagta,
attattaattggggtt, tgatcagacacaagca, tgttgccttgtcctta, ttgacttccaacagta,
cttttggggggggaga, tatgtaccaaaagttc, cgctgagcctgtaggc, aatgttcacttcccca,
gtatacaaatcgtcac, tccaatacaacgtaat, actattcttttatacc, cctctttgtgggaccg,
atccatacactcactt, tccatagccgatttga, atctgtcagctttagt, ccgttcttcttgccct,
gttttttgaacgtagt, cagccaggatatatgt, actaaaaaaaggcggg, ttaattttcgaagata,
ataaacaaccctcaca, tagtagatggtctaaa, tcacggtaaccgatta, gggcatgtcctgatag,
gaagccaatattccta, gtaccttaaagtgact, cggaaaacttctcact, ctgagaaaagtcgtgt,
ggtggaactgaagtga, tgcgttatgaagtaga, gaattcgagaaaaact, tggccccccttcatta,
tttccatgcaagttcc, tgcagttttttttggc, gatggggataacggaa, aagcagtcctcttaga,
tcatcttatcggcaaa, atcctagctgggctaa, ccactggtagaagacc, agtatcgtggggggtg,
tcgctttgctgcttag, gacgcagatgaggtac, catttccggaatggtg, gaagcaaagtaaagtt,
ttagcatgagctggat, gctagccaatatgtag, atttctgcttagctac, agattccgtctgcgaa,
taaagatggtgatacc, agcaaaactctcaatg, gcttggagcatacgaa, gggggcgaaaaatcaa,
gcctttattacacaac, tggtgagcatcttttg, tccctggtgtggttga, tccatacaaaggatat,
catcatatgatgacgg, ggagatttggcactta, ccaagccagtgttagc, attcttaaacacgaag,
aatagagaacgttact, gggcttagaagcccta, gaccgatttgcttaag, atatcgccttaaaagc,
agtactagttgtgtgg, tggtcgcatgaatctg, tcagggaaagaactcg, tgcacaccaacttgaa,
gataacttgatgttgg, aaaattgcccggtgtc, gttgaatcattgttta, atcacgtgatccccaa,
ttccaaaaaaacgggt, tggtcctgcatttatc, caccgcatgacgaggt, ccttaaagcacgttgc,
gcggccagccgtcccc, ctggaaccagggtgat, actttcggtatgtata, agtcacgtaaatgtgg,
tgcgaggcttccacac, atgctggttacatgat, tggtattgactgtaga, ctgtcccccgcagcgt,
taggtccccccccctt, agcgccatgttttttt, cctgtaggctctgcgg, tcaccccaaaacgttg,
acccttaaacacaaag, cccggcaattcagtgc, gtctcttcccgtggtc, accgtgcatcgtatta,
aatagtggctacaaga, gtctttctgatcccct, actaagtggcttatgc, ctccggggaaattctc,
agacattccggacggg, ccaccctatggtgtta, aaagcgctctggtggg, agcccccccctccttg,
gtccaccttttttttg, cttagcctaaggggtg, tgccctacaattcatc, gtagagttgtgtgagc,
tgtagggggttcttct, actctgtgggtcagac, gcactccatcttggcg, ctgctgtgcgggcatc,
agggagaccgtgtttt, gatgggaactataagc, aggaagtgaatcccta, atccactttccggttc,
gcaccgcagggggggg, ctcggcagtgctccag, aaacaactgacaacta, aattcctgcatttagc,
atatgagagcacctgg, gtgtgttcttaacgaa, ggtgctctgtaaccgc, tgtaacacttatcgtg,
tcgggtttgttcctcg, gaggaagccttagagc, cagcaagtacgatgtc, tgcgccgcgggacaca,
cccttcagaggtgacc, ggaatttctattaggt, cagaccgtgcggccgc, tccgtggctgcgcagt,
gcttgttagttaatcg, acctttgtgcattagt, tgggtggggggaacgc, atttatcttacgggct,
tattgatattgagtct, gagtacggaactcatt, tggatcttggggtgtg, caggtggagctcactg,
atgttagctcactgcg, gttggtctaaaaaaac, tagcaagttagctaca, tcaaccttatctatgc,
tcattgtgtataagac, cctagctgcggcctgc, gtacatatcttatgca, ggtaccactcaaaata,
cagtgggtgcgttcac, aggctggactgcacta, ttgcgccctgcactgc, gtgacgataaaaaaaa,
ccccgcgtggctgctg, ccagctgtgattgtca, aacatttttagcaacg, cctgccccgaaaacaa,
ccttagctcctcgcca, ttgcgccaactgcatg, ttaatcattgctagga, cgcgcattccgcgctc,
atccatggaactcacc, gccagggcacctactc, cacgtcagtgtagctc, cgttatcctgggaggc,
tccatgtatgcagtag, gagcagcattgatatc, ttgagtactcactggc, ttgcttcttccgccac,
tccccaattagcctcc, ctegtgggcctcccgt, atactcctaatactta, tctttggcgtcagtta,
ttgccttcccgcttag, gcttcaaggagttact, gggaacccccccttca, cgatcgccacatgtat,
aaggcacggcagcact, cgcccatttccctgtc, atagtgaccccgcaag, caggtgtctttatccc,
gcatattggtcgtata, tgcaggtaaataaggt, cttggggggatgaatt, gtccatcagctttgga,
tatctttatttaagag, tcagaacatccttggc, ttcctaaaaagagtac, catggtatgggggggt,
taaaagacttcgaaat, cttcttatagaccttc, gacatatagcccattt, caatcccctttttttg,
gttgcacaatctcagt, cggtatgtatatttag, cagttgcgttattttt, acttcatgctacattg,
gccccccccaccaaat, tgacagctgggtcttt, ttagttcatagggggg, tttatccctggtgtgg,
ctgctggctttagacc, actctgttattaccag, gctcagggggcccccc, tgtgaccttgtgtccg,
ctccaaccatcctgtg, acatagtgtgcccctc, actgttagcaattaat, atctcatgattctagt,
acgttttctattataa, catttttttcgaatga, tatagaccttccggat, cctgacttacgcattc,
tagggggttcttctta, cgctaactaggccgcc, gttagggtgtgcttga, cgaagtccaaatcact,
ctggcgggagcacccc, tacatgacgtacaaag, ggtgccttggaatgcc, cctctctatactcaat,
gccgtagggtcatttg, gcaggctatgcccaca, gactcagctctctcta, gagtccgggggaaatg,
tggttagaccaacaga, ctaggaattagcaaat, ctgagtggggggggga, gagtccgcaagctgtc,
atcattgggtagtcta, ttgctgttcaagcaat, ggcaacaaacgctgtt, ctgattgtgccacgtc,
agttatccggtagagg, ctagtactgttgaact, gtgatgcaatatctga, aatattttgccctggc,
cccttgtgttgtaggc, aactcttagtcaactt, ccaggctaacatggtt, tggggggggccgctag,
ataagtacctgagctt, gcttaagcactatata, taaatgcatcccccca, gtgcggtctgggtttg,
ggcctggccctatggc, tggttgcagtgcttgt, taggcatggtgggtac, actttatctaaagaac,
cccctctaacctagat, ggggggggctgactat, aaagcgccttggtcaa, actctggattagttat,
atcacatagatccata, ccatagtattacactg, cgatgaatatacagca, tccgggagggtgaggg,
taatctatgccccagc, actttccggttctgac, agcgcgctcagctcgc, ggtcggggaaaatagc,
aaaaatacggtcatta, tgggaccgccccccca, atgggaggcataggtt, ttggtagtcttgtagt,
ccacaggcaacgtagg, aacgaaatagtgttat, ttatccagtttcgggt, atactttcatatgaca,
gagagctgaataagta, cttctcacgccaaggg, gggataagacctttct, ttcagctatgcagctc,
gcgccatcgcgtggtg, cagagtattaccgtgt, tcgtgttaacacaggc, gactttatcatgtatt,
aagtgactattagatc, acgagcttagagaaga, cttggatcagaggcac, gcatggctccacagga,
ggcttaggcggaagga, gctccgcctctcctgt, ctttagagacgacaag, gctggtcttgccagcg,
tcggtagtttaccttt, aatgagtctttgctga, tctgaaggcgtatctg, gcatgttgacattgaa,
agaactgtccacgtta, gtgacacatttataca, aaaatttgaaggtgtg, cgctctgatctaaacg,
ttcgggtttggttttt, tccaccettagccttc, atatcaaaaaaaacat, aaaactacctgacaga,
catagatgggttcttg, cgcattccgcgctccg, gctttttttgcctgca, aacaacacctttgaca,
ttgcctccaacccata, ttagctgattactcaa, ctgcgttatacaattt, agtttgaacttagtta,
tagaaaaaaaaggggt, tagccttcaacctcca, gtcatgtgagttatta, caccaattagacttag,
aaacgaaggaaaatca, aactctgggtctctga, ggttttgcaggaagtg, tctgcgaatatactta,
aagaaataggggactc, ttcccctcacatctaa, ggctaggccgccgcac, aaatcaatgccaaaac,
agtgggaccaccctcc, tttacctcacactaat, ttttaccccttggaac, tctcaggtatgggatt,
gatagcaggttgattt, tcttaaattttcggtt, tggaactaacgcattg, agaatgggagacctat,
gaactttcgctcaagg, ccgtctgaaaagacaa, ttggacagtccttggt, taatacgctccattat,
cccccctggcagtttc, agagcttactcccttc, gggcttagccttcaga, tcgctcatggtgcgct,
gccgttctgaatttta, agaaccacaagacggg, catgacgggaagacaa, ggaaggggggggtctc,
ctgccccgcggcttgt, caatgtgcgctagcca, taagaggatggggtgt, cagagtaactctaggt,
ttttctcagcaatacg, agaacgcacaacttat, cttggcggggggggtg, gggtgcaccccaatct,
gtcgccgctcagcatt, agccccacttggatcc, agtccagtgtatgatt, ctcatagcctgtttca,
gccatggaacattgga, aattagcccccccttt, ggcaggttaaggatga, tcagtcttgtcatgta,
tttcggattatttagt, aagggatcatctgtta, tatctcagctcctggt, ctgagtagaaccccta,
gatggtggaagagtga, ccctgtgcggctgggg, acacgggtgtgcctaa, gcgctgggctctggtc,
tctatcgttgttttta, gggtctcttcccgtgg, acaagaattcaagtct, tggatacatcatatat,
gcgtagggccatgatt, actcgtgtactatctc, cggagttagactccgt, cgcatatactttgtgc,
cttccccccccaaagt, tgtgacctagagctca, gtctcgccttgggggc, ccaataaccctgatta,
cttgctttacgggctt, atgacacatgagtcaa, gctgggggacgcagga, aactcaagctgcctca,
cogcccccccccgagg, gtaaaaaaacttgaag, ccttgggattcctcac, cccaatctttcaacat,
gttgatcagcgtcaat, aaatcccccccccatt, agacaccgatgatgca, cttacctatccttacc,
ccctgtttgttatctg, gatcccggcatccttg, attcatgagaccttca, aagggtggattagatt,
aagccgctaaaaaaat, ctgtgatgatgagaag, gactgttggaatgcct, ccagcccccccctcct,
acttctgacaggggag, gaaattggagaatcgg, cattcgtttattctcc, tttccagcgtttttga,
agggggggggtgttac, ctgaatcccccccccc, acatcctgttgagggc, ggaagttgcccttcat,
ttaagtctatatgcag, gggacatgcccaattt, agaaattgagttggct, ccctatttccctccgt,
tctcgggaagcttagg, tcgcaaagttgtttat, tgtactcgcagttagg, gctaaccagtgcaccg,
ggagggcatgatgaaa, atggccaacacggtta, cagttctattggtgtg, tttcacctcagcgctt,
gaacctttaaaacage, atttagatacgcccca, tggaggcgccgttccc, aaagtactataccatg,
cccttggtatcttaag, gtgtgtccggaacaca, aaccaagaacaacggc, cgctaggcaccgcgcg,
acgcttttttttaacg, cgacagattatggcgt, gagtaactatgtaatt, tcttaatggcttggat,
ccaacataaagttagc, agtggagtggggggat, agaggaaccccccacg, atcacataggggagac,
gggctcttcccaatga, ttctcaccctgcccta, cattaagatatgttac, ccttatttattgtaac,
acaaccccgaggtatc, ccacttgtacgatctc, catagttaaaccctat, atctgatagaacacga,
ctgtgacggcatcctc, cagagctgaacgttca, ttaatttaggcagcta, tgtgagatctactaac,
ttttaaaaggacgctc, ttaccaacatacttag, acatgtcttccacgtt, tcttggagttctacca,
ctataaatatctgcct, tcggggatggagtttc, tcacaaaaagaacgga, cttcccagcttagagc,
cccatagccttctgta, aagtcaaccgaaattc, gggtagccttactctg, agtctccatacccggg,
caagggcaatctgtcc, cagcagcggcgggaga, attacagctattgatt, gttaggaatatttcca,
ccgccacaagtatgtc, aattgttccaggatag, gatgcgggttctggac, gcttgggaacactgta,
gtaactgttacacatg, gctcatgaaccttcat, tatgcttttactgggc, cgtgcgccgcgggaca,
taactgtataaaagcc, cagccctactttcaag, ccctgtttcctgtacg, taaggcagtttgtctg,
gcgattctctattggc, cccccccatgaattgt, cagcggcacgatgtcg, tagtttgttagattag,
gacatctccatgtcat, gggggccccccgaggt, gcaggtccacccgccc, cgctoggaaatcataa,
cccggctgtctttcta, gaataaatgtgacccc, tgatccgctgtacgac, tctcttaggatagttc,
gtcagacttgattata, agagtgggtgcctttc, ttgtaaaggccacgtt, cttgcttaagtgtgag,
cgtctcgccttggggg, gagctctgaccgattt, gcgggaggcctggatg, ctccccatcctgtagc,
ttcccatctgcaatcc, atttctttttacgtcc, accgagaaatccattc, taggggaggtaaacct,
cgtacacccagcttta, ttttccccccccaagg, cgggggggctaacaga, agatagagtgacttct,
aaactcgtgttatttg, gtctacactcgagaga, tgctccccagtggcgc, caacatgtcagctcac,
tccttggttcagtgtc, aaccttgttctcccgc, gcgaccctaaggctgc, ccataaccaactagaa,
gctttagtcccagtta, aagtgggggggggaat, ctggtgcaaacttgtt, ataccaccagtgccgg,
ggacggcccgcgctag, cctgacgcttctgttc, gggctgactgagccat, ccgccctcattcagga,
cgtaggctgggaggag, tgcagcatgcattcga, gggcctgccgcccatt, aagatcatttccggaa,
tactgcttggaatact, cttccagcaaaaagac, gagggtctagcctttt, gtgctggtggtcgcat,
ccaccagtaaacgcgg, aggagattatggatca, cacagcccctgtcgac, caacctcgaagggatg,
tggttaaaccccattg, accgaagaaaggagcg, ttaagcatggtcataa, atcaatatcttcttaa,
taatcactggccgttt, ggggtagcctcctcat, atatagcttgtatcat, ctgttgcaaattgctg,
gtgcgggcatctttgt, ggtcctacaccatgtg, atgtgaaaatacaacg, atagcatgggggggga,
ggatatgggggggtta, gtgggctgataatgag, agagttgcacccgtgc, cttcccagcgtgcaat,
aacagctaggcggggc, gacaagctagattcgt, tgaatcagtctaagaa, acgtacatatagagac,
ctcggggtttttttag, tcatatgatgacgggc, ccctgaccaagttagc, cctagctgggctaatc,
gctttttttaagaccc, gtgtgcactgcaggca, cgggcttagtccaaaa, gtgatcataacggcaa,
tcattttacgcctttc, aacctgactattactt, taatcgttggaattat, gtatgcgaaaatataa,
ccaggatgggggcttg, atattctcttgatggt, gatatacgtaaaatag, gggacaaaaaaaacgg,
ttttcagcaagtacga, cttacttggacccttt, gacagctgatacttgc, cagggacgatgcattc,
acaggcattagccgtc, cacaattattccagga, tgcccggctgctgcaa, accttacccagctcga,
atgttaagggggggag, agattagccagcttat, gttaaggaaaaacact, catcaagctccgatga,
tgagggggggggtgcc, acgaattatacttttt, gggactagttaatgat, cttaatgcaccttaac,
atcgttcctccctccc, gagttagaaccaacta, ccatccttgctacatc, ggggggggtctcctgt,
tcaaatttagaaaccc, attagtagaaccctct, caggttgcccaagtcc, aggagatccaacctgg,
tattgttggtgactcg, ccgctgtacgactcca, gatgtgatggcggcct, cccacccctagatggt,
ttgaactgtgaataca, aaccgtttttttggta, ttgggaggcgcaagga, ccttgtgtaaagctta,
ggctctgtagttagaa, gtcggggggggcgggt, tcgaacagccaagcag, tgtgacgttaagtcct,
agctgcaaaccagatc, aacggggggtaaatat, atcttagtacgaatag, gtaccaaggagagtat,
atctatacaagtgtcc, cgctgctgtgtcctga, cgaaggcaagatggga, cggaaaaaaaaggcat,
gagggcggccttgccg, gacgccggagccgttg, ctcaccgctcaccatt, tacctggctcagagat,
tatagtgaagtacctc, atgttgttaggttcta, agtgtgtccggaacac, tcttgacttgattgtc,
ctagcaaaaaaagggg, ttagctctatctgaga, gcgttcagggactgcc, acgctcaatattcata,
ttgaccctgttgctaa, gaaaattaatcgacca, ttttttcgtttggtca, tgggggggcacattgt,
ccttgttaacttgtgg, acggggggcaaaagac, cgtcccccccccgaaa, cgaccagtttttttta,
tgcacttaggtggaac, tagcgtttcctacagg, ttaggacataatgtgt, tgctacctttatgctc,
acctccaccatgcccg, tatgaaggttggggtt, tgcccactctcgtggg, tgcctcccccccattc,
gctagacacaccttga, ggctccagaccgtgcg, gcaaactgatgcatgt, gtgcccctgagtacga,
caagctgttttttaac, attccatctcagcggc, gtgttttctgccgttc, cccagcttacttcgga,
atcacgacatcagcgg, taacagagcctgtaat, cgccccgcttatttta, ataatcaggatgagaa,
tttgctgcaggattcc, gccagattattatggt, tctcctggacagttcc, ccacatttatatcttg,
tgtgtgcgtgcgccgc, tgcgggtgagaaaaga, agcacataatgcttta, catgccgggggctccg,
ttacaaactagcatgt, tctttgggctcggtgg, agtgataagtctatgg, ccttcgtctaactcta,
ggacccccccacctgt, cttctcgtttggccag, aaaaagacaccgtgag, acgctggacccacggg,
ttcgaagtaaatattt, gcctggggtagagccc, ttacttaccaatcata, ctgtaggactccgttt,
aaaactacaactccga, gtgaggtgatcacatg, gggggagataatgatg, cgctgttatttccaaa,
taaactcccatagcac, catcaaatcaagcagt, gggtttacgcatcccc, gtcggatgtgggattt,
aagtaccccccctttt, gagacggggggggtta, gcataaacagacaccc, ggaaaaaaacgtctag,
cttaggcaggcatatc, cccccccccaagggag, gaagcagttgggctgc, ttgtgacatttgtatg,
atgatgcatatcatgg, gcaggtgcataatgat, cgaacagccaagcaga, aagactagggcggttt,
actccatcttggcgac, ggaacagacgtgatcc, ctagcttctgtccata, caatcttcgctttatg,
caacacccctttaggt, tgctaatgttgggaac, ggcatccaccttacat, cccagctacgccggcg,
gggatgcaaacactgc, tgagcacgggggagcg, cctgttaaaaaaaacg, aagatctgtgcggctg,
ccagaggctgactaat, gatgatgcagaaaggc, tagttaaaccctatct, atgttttgccaccgta,
ctgtcctgcttgcgag, attccgcagacacgcg, tagaatagtcaatccc, gcattatgtaatatcc,
cttccagcccttttcg, ggatggaagacatggg, cacacttggaaacaag, cagcggtggggctagg,
agagatctccttgtag, ttgcttaacatactga, cacttgccgcagtcag, gggcaagtggacggag,
gcgttcggcctgtgat, cgatgctgtttcacca, gtggtagtctatactg, cactttccggttctga,
cgtccccagggttcct, tgaccctcagtgtttc, tacttgctcaattcct, atcaatgcagggcaaa,
ttcaatctattcgtgc, ccagaccgtgcggccg, cgagcatctggaacta, ttgtgggtgagaggct,
tctgagtagaacccct, ttagccagcttatcca, atgctgaagtaagacg, gaacctttagtatccc,
gatgctggtaattgat, gatttaattcgttaag, ttagatctcatcttga, ctcccccccaaccaat,
ttggatgtacttacat, cataagttccaaatcc, gctttacaatgagtct, aatattcctatgagca,
agtaattgtcagtggg, tgtagtgaaggctggc, tgacttatataatcag, ttatattgcccccttt,
gattgggccccaacca, catgggggggaaaggg, ccctctaacctagatt, cttatctgaggcagcc,
gtgagtgcttcactcg, agtagagccagtgata, tcgaatgtccatttac, gatgagacgattgatt,
gaaccaaggaggtgta, gtagtccattagagca, ctgctttaagcctcgc, catgggggggataaaa,
tgtgccttagcaaggc, atcggtgatttgtctg, ggcgcccccccccagc, cgagaaccatctgaaa,
gtgggcgtgtgcgtga, aggttatcactgtgtc, acgtagaaatttggtg, ttaggggggggacagt,
atgactttagacttaa, gggagatggtccaaat, cagggtcacaatctag, aggttgggcaggcctt,
atacgcatctattttt, ttcttgtttaagtgct, aaggtccgtctcgcct, tgtgaaaatacaacgg,
gacatgttgtaaggcc, ggacattccacatatc, aacgtaaaaaaaagcc, agtgcatcatcaatag,
gccatgcaatgggtgc, cctgttagctctactg, gcttccacccatgccc, ccgaaaaaaattttat,
tgaaacgactgttcta, ccagtaggggcttact, tcttggctttgataga, tgcaaatagcactcgt,
tgggaagcttaagcac, ttatggcgctgagcat, atcacccatctactct, gtagccactaccttga,
gttttagggatggtgt, atgcctaccaagtttc, cctagatggtgggtcc, agtttttgagtgatcg,
accttttgggggggta, tgcgttgcgcacttcc, taacgcttttttttcc, catttgtatgcaatgg,
gcacgtgttatgtaat, cctacttgacgaatag, gacacatggcattcgg, gtgtgaatagaccacc,
acatcctccacaaggt, tcctcataaatccaac, gcgtcctagggattga, tattcgatgttggtca,
tgcgttttttttctgg, cagattcgtcacaatt, ttgtagagtaggaatc, agtacttagttatctt,
tccttttccagcgaag, catcatctttccgttt, gaggggtagaattatt, atgaagagggatcaag,
gactttgagtaaactc, ggtatctcgaatggta, ccctttgatagagggt, gatgcctttatccatg,
caggtgccccttatct, cccaattgtgagctga, tacatgtgcaaaaagc, gccgtgtggctgagag,
aggtccactgccatta, ctactggtccaagatt, ctcatccttgatgttc, ccgatacgagtctccc,
cagaaaacagtgccag, gccacttttttttggc, actggaatctggaaat, tctttgtctcgctgaa,
gaacaacggcctccca, gtacattgaacaagaa, aaggttactagttttt, agttactatctcattg,
tacggatgacctcgtg, aatagcttaatgctga, agccaatgtggaaccc, cgtcctcatcctccta,
ctattaaacacgctgt, ctacccatcaaatatt, tgttcttgtgcctaat, gcgcaggactatccaa,
gcagattagccttcag, aacacgctgttggata, cgctcctttaagcagc, gggggtaggggggggg,
cggccttgcgaccgcc, gggcattcatcatagt, ttgaagaacattatgg, ctttcactctcgtccg,
aggtttcategccttc, tgaaagccccaacata, tccccaaggatttaga, taggggggggctgggc,
tttctttttttcgcaa, gacgctttgagcccag, tgcttcccactcttaa, gttagacccaatcaca,
tgggttcatttggagt, atccgctgtacgactc, agccccccaaactatc, acctgccgtgccggat,
ataaaatcgcagcctc, gagcgtaggaggtagc, tttcgagagtctgact, gctccaaccacatgtt,
tacatgccagtccaag, cttgagtatattagct, cgtcaatgccaccgcc, gagactcaaagtgatc,
agctggaaatgagcgt, tcgttttggatatata, cacttcgtgggggttt, acggtattttttgtgt,
gataaacctagtacct, tgcgatcacctgagta, tatctatcccgtagaa, aattcaccggatgggc,
cccgtgcccccccccg, cctccgcatggtgcga, ggttgctgtcgacctg, ccgtgagtgctcagct,
ccgagaactatgctgg, atcctaatctgataca, aacgccttttctcttt, ttaagaacaatatgca,
agcgacacgagcgaaa, actgcaacaagtattg, cttgctgtagaggcca, ttaatgagagcaaagt,
ttatgaggagcgtgaa, tatcaatcagctaatg, ccgtaaatcacttgaa, ggcggtgaagttcata,
ggaaaaaacggggatt, actcatgtagatagca, tttagtctgaatccct, ttctaaccccccggtg,
tcctccacatagggac, aatctgcgttatacaa, ctcccaaccttgggtc, tgttataatataagtg,
ggcaaggggggatatc, ttctgggtagagccgt, tgggatagttagaaac, gacttaatgagtgaat,
cctattagattatggt, catcgtacagtgggct, agggcaaggctcggaa, cggcccagagacagca,
cttttttctatagtcg, atgtagttatggtgaa, aagtactcatcttaaa, ttcaaacaagagcggt,
aaggggaaatacccat, ctgttatgtaatatga, tcttccgccactagat, acaacttcctccattg,
aacccatgttagagtg, gcttaattgggggtct, cattgggtgtgcattg, acgaaaaaaaagggag,
caaagtggaagtctgt, cagatgcttgggggta, gtgcataatgatgcta, atactcaacctagggc,
gtccccactttcgtgg, gatcgaagccggacct, gtattttaccccttag, taagtgattgttacga,
gacgagatattgcttt, ccggggtgcatcgtgc, aatcgaagtagacagg, agatggtatcccgccc,
gcgccgggcgcattgc, aattagaccaagcctt, gtaacccagccatgtc, agctgtacagctgtta,
taccgctaaaaaaaac, gaccctccgggagggt, ccttattttgttgtgc, agcccaatgtgacagg,
tctgctatcctcgctg, tgcgaaaatgttaatt, ccctaaattaccctag, cttaggcgggtggttc,
caagcaattgcacctc, acacccaccgatgtgt, tccggtgctgacgaac, ttacgaagaacacaga,
gtttggtagcattagg, tctcgcaaagtgtcag, tagtcaagacgcagag, gctagaaggattcaag,
cgagccttgcgagggc, gagttaaactacgtct, aaacataatactctgt, ctcggggttgttgtgg,
caccggaaactgtagt, tgaatgatgtcagctt, actaaacaatatcatg, gatattccctctgaga,
aaaacttcacggtgaa, agtcccatttttttgc, cacatgaggagtcagg, caaagaagctgaggtg,
ttccccctcgtgcaaa, ggtcatggcctcctgt, tggcacgtgttatgta, aataaggtgtccaatc,
tctcgtgggtcatctg, tggccctatggcccgg, gttgggaaacttaagt, attcccatttgaacct,
atgggggggggggaat, gagccaggtggcaaac, cgtttttgtactgatg, ataggcaagaaatagc,
gtgatgcgagattgcg, gctttgccagggggac, ccacatctaggttatt, ccatcgtacagtgggc,
ggcgctttccagtcct, cagagtaagttgtgga, ttgcaaacctgtcttc, atcccttcacctcgac,
ttcgaaattagggata, acgttttacccagagg, ttacggaagacagttt, tcctacgcccacacgt,
atgtcaggataggcca, atgcacgcagaataat, gaggaccagtggtaga, ctcgaacagccaagca,
ttgctttaagtgttcg, tttatctcatagtcct, ccgcccatgcagtaac, gttcttgccactgtta,
ttgtcctagagggcct, cagacaaaaaatcctt, cgtgccatgtcctact, tgcagtcctattagcc,
tacagctctcacttct, acacagggccccccgg, ggggtagagccccaac, actccggtgacccttc,
taattctggcacgttt, actaataggcttcacc, tgctaggaggacttca, cctgactcagattgcc,
tcttctcccagcggca, gatcagatagagtgca, ggctgtggttacagcg, cacttcatccttaatg,
ttttgggatcacagat, ccaaggtgccatcctg, ctcatgtaagtattag, aggcagattagccttc,
gtaatatggctgcatg, cagcctctattagtgc, attcacctttatgaga, tgttaattattacaca,
ggaagtctaagtccgc, cgaaaagtgtttgtca, ttatgcttactgcttc, ctaaaaaaaacgtccc,
atacaaaaaaaggcgt, gcttaaactggttatt, aggattctacaagtgg, ccttaaagtgatctat,
agggaagcttatttag, caaacgttatccttgt, ggacagtcccaaccac, gtagcctttacagcac,
atagctgccgacccct, tttgggatgggagttc, tacagtgcagctacca, ctagcgcgggtggcgc,
gccctggcgctgtgtg, catggttaggctgttt, gcagtcccatctgggt, tgacttatcagactct,
tttcgatgaaaaaatt, ctgagaccctactgag, acgtacattatagagc, tcaactatgagcattc,
ctcaatataaagaggt, gaagttggtttgctgt, gcagtgcttggttgtc, tcgatcattgttttgc,
tctcgtctgaggcttc, ctacgaggtatttaga, cctctgatacttccaa, gtagcctttttttggc,
acatctgaagatacat, gcattgcactttagct, acaggtcctttttagt, atctacccaagggtac,
gaccccgcccctaaac, gagtcacgtaaatgtg, tagtgtgggggggggt, gtgaggggggtatatc,
ggcatgtggttaggtg, ccaccactccccccta, aagctttagatctaag, tttaacctttgcttac,
gggttcccacagtcta, aatctctgttaaagct, ggagattagttgtttg, accccgaggggtggga,
tctatcgaaacaaatt, tcgttagtgaaacact, aaaaagcaccgtgaaa, ccaggtaaatatccct,
agccctcgtggaggct, catgtaaacttagacc, atagggtggagggtta, agtgtctacaccgcag,
caacatgactcaaccc, atagctctcagtgccc, atacatttttttgtcg, ttggtcatagccagct,
cttttaaagattcgag, ggtgggtttccatgaa, tattctagatagcaac, gaagaccaaggtgagt,
cgcacagcaatacacg, atagtatgtcccatta, ataggtcaactataga, aagttaaggaggcttc,
aagggggggctgcttt, ctcggatatccggcac, ggcagccaggcgtcta, gacatcttatgcaact,
aagccctcccccccca, ctagttgtggacctgg, gatatagccccccctg, tgaaagtggtgtatat,
actataaggtgctttc, ccactttccggttctg, caattagcagggctgt, gcagccatatccccaa,
ctccgtctgaaaagac, tacaccacgggatatg, atgaaagtgtactctt, gtgttgcttgttgacc,
cgagctcaccctccta, tgtatatggcctcgtt, ggatgttgtttaatta, ccggcctgatttgaaa,
ccaggtgcaacaatgt, ggaattttttaggggg, atctgcgttatacaat, tgtccccccacccgaa,
gatacgtgggaagtgc, ctccgcaaggaatggc, ccaatatggccttgta, taggaatgagcaaatc,
gttgaattattcccca, caccacagtacgtggt, tcctcctgcctaagta, tgacaaatacaccgaa,
gccctacacaggctgt, gggcaggttgacccca, gctcaaaaaaaaatcg, cttatgggcagcaatt,
ggaacgtgggggtctc, aacccagtcttacttt, gcctccactctagagt, gagggctaggggggca,
atcgtgtgctgtggga, ccagtcctaaaaaaag, agtccttgtaagtttc, ttgttacacccttgga,
agcctttacagcacaa, aggctacggggaccga, gctctcttctacaagt, caccacgggatatgct,
accttaaggtacaaca, tgcttgtctgaattag, gcaccttgtgccccgt, atgagagcagggtgta,
tacggtacaccacggg, gggaccaacaatctgt, actttcttccgtacaa, tatgaaagggagtcat,
ggggttttgtttcaat, gtacgcgcatgaaagt, ttaagacagcatactg, cagacttctttccgga,
catcgccacagctaag, aaatcacccccccaac, tccctcttgttaataa, ccaaaaggttggtgat,
gatgcctcctgggtcc, acccgccccccccgaa, cactcggagaggccgc, cctgttacccttcctc,
agctatgtttatctga, aatactcagctatctg, gatcctgctcgcctct, gccttaaaaatcgtgt,
aggtcttttttttgcg, caggcatagtaacttc, ctcagtaaagcctcaa, tgtgggtatctatccc,
tgaatggttatttccc, acgacatcagcggttc, ggtctccccccagcag, ttcaggccagagtggt,
ccagatatgtttattg, ctgggggagtaggttc, cacctccagatgtaca, aaaaaacggggggtaa,
aggtgtatgttaacat, gcatacgtcagctttt, gttcattattccccca, cttgcgttttcacctc,
ttcattagtccggttc, gcattgatgacagcac, taacacaggaacctta, ctgatgcagtcttggt,
gcagcactgtcttaca, cctaaggctgcggctt, gaagaactcttcttaa, aaagcatccttagagc,
caaatgtaaaactccg, ttatttcggaaacatt, atgcatatgctaaagt, aaagacaccgtgagca,
gtccccccaccggaaa, gaatattgagggtagc, aggatttttgtcctcg, tctgtgttttaaagcg,
ttaaaaaccataagtc, gcgcagggcttgcaag, gttgccatcatcacaa, gctccttggatattac,
ccaacttcataggacc, ccctgatcaagtccgg, gacctcccagaaactc, ttactcacaatctaca,
gtgtgcattagtcttt, gcccccaccaatagat, ggtatggtccccgccg, gaggtgatcatgaggc,
tcgtattaacaaaagt, tggcaatgcggtaaaa, cataacctacctgtct, gtaatgtttctaagcc,
tgcccattctcttgtc, cgggattttgggaggc, tccccatagtgacccc, ggagacggctacggat,
tcaaactattcggttt, cgattgcaggcgcgct, ttaatggtatctcgaa, gcttagttcctatgaa,
actacaagactgtcca, gtatcagcacactcat, cttgtgaacttgttgc, cttggggttggacttt,
tggttatccatttaac, ttgtcatctgtggtag, ctttggcgtcagttat, ttcgagtacatcctcc,
caagtgaaatatcgtc, tctgtactctactctc, gtttgtgagctgtaag, aaaacttatcggagga,
aatattgctcaacccc, tctgataattgggaat, tgcatatccatgagac, gttcccctccgtatgt,
cagcgactcaacacat, gtctttcagatcctta, caaaacaacgataatt, caggggggttggggaa,
aagttatcgtcaaacc, cttgctcttacccaat, cagaaacctccccatc, ttgacgagcttagaga,
ttgtaacttccgatta, ctcatgattcgactcc, tatgggtagctactgt, gactcctctcgggggt,
ttcggagatataccca, gagggggcggagcgct, cccgcatccatggata, acgagctgggttcaga,
caataaccgagaagat, ccacccccccccatgc, aggtagggggggggtc, ggggatcattgagatt,
gtgcactatgtttata, gggggggtaagctgtc, gaactttctaatagac, tcctggattaactttc,
ttggccggtcgctgtg, gtgtatgtcatagcat, tgtatttgtgtcccac, ccggagtagggacatt,
gcttccttgattagcc, caattagacttagagc, gccggctaggccgccg, attgggtacaatctat,
tctattaaacacgctg, aggacatacttgaaaa, ttaggggggggaggag, gtgtgaagatattatc,
catggcacatcaaggt, actgcattggatgtac, gtttatcaccacatcc, caatgccaccctgtta,
gggcatagcttaacaa, gtcctgactgagcgtg, tgggcagctgaagaac, gcgttttttttaagtc,
gaatatacctaacctt, atagcgctagcgctat, aacttatgcactatta, tggtgtaggccgggct,
cagtacagtgacttgt, tccagttcatagatta, tcatgtcacgggactc, gttactgaggtgctcc,
ccctaagacttagagt, actataattctctcag, ttgctggtaaaagaat, gtacccaccgcctccc,
actgccccccagagat, accatgacggcttact, ggctagtttcatgggc, tgcgctcaaaatatat,
ttctagggcgaaaaaa, ggaataaggagctttg, catagtattataactc, atattcatcacgtttt,
cttggtcaccgaatct, gtctggaggcgccgtt, gtggacaattagcgcc, atttagtcaatgatta,
tgacagagcgttactc, tagggcggttttatta, gctctcatacccttcc, agactccttacctggg,
caacgtaaaaaaaagc, caagtgtctagggagg, caccttgacctcgcag, ctttttttgttagacg,
gctctgtctgttcaag, agtccatgagacatgg, ggatgcaagggcttga, ctgttagcctttgtgg,
gagatagtcaagacgc, tacaggctatctttat, ctgccgagttattagg, tgaatatgatagccgt,
ctaactgtcctacatt, ccgcctgcccctaggg, agtttgggccggataa, ttgaggaaggacgcca,
cacaataccaagcgag, acctcctccataatta, gttgtcgtatcatttt, tttttaatcgctttaa,
gccatgccccgtgtag, ataatcactggccgtt, accggctggaactgcc, ctcctcactcggcggc,
tatgcaatgttctccc, ccgtgagcattgggaa, cgggcagctcgtgctt, gttggggctgttttat,
ttgctcggtgctgggg, tataaaggcacactca, taacactcatcaggct, catctatgccacgaag,
gacgaaagtgagtata, tctcactagagcagca, tctgcccggcacttcc, gacacgagttagactg,
gcatgttagcgaatta, tactcgtgatttaaaa, caggatgggccaaggt, caatataggtaacaga,
aaaactgtgcgaaatg, gcacttaagactgggt, tactcacccaacactg, gattctgctgctatta,
tctgtccacattggct, atgagtgcgaacgtgc, aagccacgtcctcttg, gcacaggggagactta,
gattaaatctcaggcc, tggctctcagccaaat, cccatggcggcccgcg, gaaaacgccttgcaat,
ctacacctttccatca, cctcgcccggccgcaa, gattattgagtgtgtg, gggggaggctccagac,
attacagacgttagac, ctccagggctgttaga, gcagtcctaaaaaaag, gtcatctggcctgccg,
ttggacaatttgtgat, cggaaaactccgcctc, taagcggtgaggatgc, gttaactgctctaggt,
tggttaaaccccgtca, agtggctgctcttaca, aaaaaggccgaaaagg, agctgtgtgtatcaca,
ataaaaaaaatctcga, tctcccatcatcatgt, gtgtgtgttgataata, tacctgttgtattaag,
accccccccaaaggct, cctcaacttaatcttg, ggggtactctgcctag, cgacacgtgcacatac,
ctattcccattaggct, gctagcgcgggtggcg, tgaggatgctgacccc, cctcaggcatgaaaga,
taatcctccgaggaaa, tccatagtattacact, tgatcatcactggtta, cagccccactaaagcc,
ttataacggcagttag, ggggggggtaggaacc, tgaggactacgaaaaa, tgcttcgctgagcctg,
gtcccggatgtataca, ttgcgaccgccctggg, ttagggggggggtttt, ttcgatccactaacat,
agtttaaagtgctcat, ttcaatatactcgaat, aagttaacccccccat, gcagttatcgacaagg,
ttaggcttaagtaaac, gtgttcgttagatcac, gcccctcgtctgagat, ccatggggggggcagt,
ggacagttgcaggact, ctcctagacaacttag, agtagactagttctat, ctaagggggcagatgc,
gccttgccgcgcgccg, gtgacggcatcctctg, ggataagacgctgagg, cctcgaacagccaagc,
ttgctgaaaattctgt, accggccttgcctgcg, acctttctcgtctgca, ctccaacgtttctgag,
tactctacaacctaca, ccttcggctccagaac, gaatagcttacatgga, aagtacgtgaccagct,
tatagccccccctgtg, agtttcgtcttatcga, cttcaagaatcgaggt, tgcgtgttctcttgag,
gacacatatcccaacc, gatattctcttagttg, acacctttgtaaaggc, acttcccgattgaggc,
gggggggcattcatca, tggacatcaaggcctc, ttgtgcgtactatatt, tataactaagtatctg,
atttcggagttgtttg, agtcattgaggctgcc, cataggtcaggatttg, gtctctcccttattag,
taagcatgccccccac, tgtctattggattctg, tgcacgttactagtct, cagacaaaaaagggac,
attagtgaacgcaaag, agtgaggcaaacctag, ttgtccccccaccgga, cctgtatgtattacat,
cccgaggtatcaagcc, tgtgtaccgtctgccc, agattatggatcagtg, acaaaattcaccagcg,
ccttgtaatcttcctg, ggtcattgcaacgtct, ttgcacgaaatttctt, gggtacatgcactgag,
gtatttttttaaggcg, agctcccccaagggca, cgtgcgggtcctgatt, aagcttagtccagaga,
taatttggcattttgc, cggggggtggagaata, ttttactgttacgaga, caaccactcaggccct,
tcctcatgccaatggg, ggtacataaaaaaagg, atagactacataatag, tttgaaaacatccagc,
cttggatgctttgcag, gatctagcccaccccc, tgtcgaaaagtgtttg, acttgtggatttgtca,
tctatttcaccagggt, acagacttttttaggg, ctctttggcgtcagtt, cctcattttttttgac,
tgtcgttgaactgact, gaatgttactataaag, ccaaccccccccaaag, atgcacgggctctttt,
cttgctgactgctgta, agccgcatcccgctct, ttgcgtgcctaaaaag, catgtatacagacgtt,
gcttcaagtcacgtga, gttgaaatttttttgg, gcggggctgtcccgcg, gcgaccgagactcctt,
ccaaggccatgatagc, ctctccggattctaaa, atcctgccaccgtggt, ctccgactgttggacg,
taggggctccacggct, aacagccaggtattga, agttatacacgtccca, aggccgcgtagaactc,
gttctgaggctcgcac, cggctaaaacggttaa, ttgaccagaagctgat, attagtcaatgaatcc,
acttagtgaatcctgc, cttaaagcccccttat, atgcaggatcgtgtgt, aggatcactgggggaa,
gcatcatacagatacc, aatatagtgcctcgaa, atccctttttttgagt, catatgagagcacctg,
cccctttggagtacaa, tgcaaaaaaatagcgt, accaaaggaatacact, tgtccagggtaactac,
acacagttaaacccct, gtgaaatatcgtcaaa, ttaggttgaccgattc, gtgctaatgcctggcc,
ctccttcacgcttaat, cccacaattttaagca, ccaccgcagtgtccag, ccgtggcattcacacg,
gtggatatagaccaca, gtatattatagcccta, aggccaccgacaaccc, ggaaacatgtgcccac,
caaagtgtgttagcca, ggctgatttttaacac, tttcgacccctacctc, caccactcttagctag,
tggttctgagtagaac, catcactaccctttgc, tgcatcacgtgatccc, acgcgagcccaagtag,
tactatctcttttcgc, cagatttctcggaaga, agtccacggcttaaag, ctagtgatctgggttt,
aacagtaatgagataa, atgccccggtacctta, taaaaggagcctacat, caccaaagactcatga,
tcaggcagttattcta, ctaggatacataaagc, ctcagtaaggaactaa, cttagagccagattgg,
cacaagctagcctcag, ggggtatgtattatga, cacatacgtggtgcgt, atctttttttaggggc,
tgtggtcacgaatgca, tgtcagcggtgcttaa, taataggggcattaga, acagaactgtccacgt,
gtcagacagctgggat, ttttcaccggaaactg, agcacttaacgaggcc, aatgaagtattgcaac,
aatcttggggggggtt, gcctgatggtcaattc, gggatagtgtttagtt, ataatgccggcagtga,
acactcatgtagaaca, taagagccctcggttg, agcttaattgggggtc, ataagtaaagctatcc,
agaaccttgtctaacc, aacagcccatgatcca, ttctgggcgtgagccc, aagcctatctaagtgt,
ttggtagcattagggc, agcttaaaggcaaggt, cttcccccccacttat, ttatgcactttgtgaa,
gctggggggtaaataa, accaaggaggtgtagg, gtgcgtttcctttctc, cgatacgagtctccct,
acacagatgaattagg, aggtatcccaatgtac, ccctcgacgccttttt, tatgaagtgactagta,
ggtcattgcttctgat, tgtactgccttttgcc, aagggggggagaggct, ttctactagacttctg,
tcattaataccgccca, cagctgatgagttcac, cactcaatatgcagtt, gatactggttgcaaac,
tacatcccagggatta, gtatggagatcaaaat, taggtcggggatgggc, ggacacgaagaagacg,
tttagcatgaaattct, tgtatctaaaaaggat, gtgcatgacatctgca, aacatgtctaatgatg,
acaaactgaggcacga, gtatgagcccataaaa, caaagttatcctttca, gcggtgaagttcataa,
tctgaaaaaaaacgga, aagcttccatgttcca, agaagtttggcgtgca, tgggggggggtgtatg,
tccggtaaactagttg, gccctagctggaaacc, taactctcacagtgta, gtaatggggcaggcaa,
agaagagtctgatagg, ccccgaaaaaaaacca, ctccctggctacttca, ccttttttggtatggt,
ctccccctagggtgtg, gattccgtctgcgaaa, ggacatacatagtgga, gatatggatcccacat,
ggcccgaggcagatct, aaacgcttttttttac, tagcgtcactgatctt, ggcagctgctataaat,
ataggttccctggtat, ggttatgtcacgcatc, aagtacattcacacgt, acaaaggggtcatgcc,
cgacagcccaagactg, tctttttggtgggctt, atattagacccattta, gatgcgggtggatcaa,
agttttttttgggagt, tggccggtcgctgtgg, ctgtggactagatact, gttcaaaagcgctttt,
cctgtaacctgaagtt, gttaccaagatttcgg, gaagaaagaaggaacg, agctgatccagtttca,
agctctgaccgattta, tacaaatgagggactg, ttacagttatagtgga, acctactttgggctaa,
ctcgctttgctgctta, agaacagctcataggg, ccgcacagtgccctcc, catccagccgtaaatt,
ataactacgtgatgaa, ccattgtgcctcagcc, ctcccttagctctgga, gtcatactcccctatt,
gccctcaaaatacaat, ccctctcgtgcggagc, gttgacaagattgaaa, tgcgtgaggagaaccg,
aagttatattatgtct, gtatcggttttagaag, cagtccctacaattgg, tttatgttagcatgcc,
aaacttcacggtgaag, tccacagaagcaagga, gcgaaactagagtttt, tgccgacccctccaaa,
actgtttcccctcttc, ggtccttgatggtgac, gtaggaaaaggcatta, atgtaacagttgttct,
ctggacccacggggtt, cgtgagtaggtgggat, ctgtctcctaagagat, atggtccgaacttcct,
ccgacgccggctaggc, accgggatccccgtgt, gcaaacagggattagg, caaagaagaatttgcg,
cccacaagactctggt, gcgctcgcgccagcag, tctaccctgaaactaa, tcacatgaaggcggtt,
agttctcttaattgcg, aggcctaggttgtgtt, actttgagtaaaggtt, cttaggtgaatgtgtc,
cttcacggtgaaggac, ttcgataaggataatg, cccactcgctctttga, gtgagggggggttgtt,
gtaaatgaaatcacgc, agctcatgacggtgaa, gacccggataggggga, ccacatctgttcaaac,
gcggactgtacatgta, acactgccaccgaggt, gcgcccgctgcaatgc, gggatcatctgttagt,
atctgtcaaaaaaacc, taaggggacaattgga, gctgctcggaaggctt, tggtttttttggcaga,
ccccccccaacatggc, gggacttatgagtgag, tagaataattagacct, agaaattacccccccc,
ggttgggatgtactta, catacaaaattaagcc, agtcttcttaagacgg, gtccactgtctgacac,
aattaatcgaccataa, ggtctgactgcggact, aagagcttaattgtca, ccaataccccactctt,
cactgaattagattcc, caacagttctttgtga, gcgctattttttagct, ccgttttttttagctc,
gtaattgaactagtag, gcgggggggcctttgg, aatatgaagaacttgt, cctttatttaatggta,
gttactcgtgcctgta, ctaaaaaatcgtgact, gaagatagagcactct, taataggaatgatacg,
agtgtaaggctaagtt, attgaagcacttgtat, ttagcgcagctgcaca, gtataaaaaaacattc,
ttgtcgtatcattttg, cgagtatgggggggtg, ttgttccttactggag, gtatattgcagctcta,
accctccacgacgccc, agcggaacgtgggggt, aatatgtaaatttcgg, ggtcccccccgaaaaa,
ggctggatggggcacc, aacctaatgttataag, cattggacatatatac, tttcctccgacagatt,
attgcgggtgagctga, ttgtgagtacccagga, tccgagggcggccttg, cccactgacactcctt,
aaataatactctgtcg, aactccgctgactgcc, cagtgtcgccgctcag, ttgagcatgggtgctc,
ttgtacacccctaaat, agtatataaccactgt, tttacttcgagtttgg, ctacagctctactaaa,
ttgcgatggtgtgata, ctttcctgcagtcgtt, taacgagaactggaca, gagagaattcgtgaca,
gatgcacttatactgg, cagtcagcttagatca, ttagatgccatagtac, gacacaccacagtacg,
agggtgttaccagatg, ggaaaactttacgaaa, gtcgctcccaaggcca, cagcgcttctctgaaa,
tcactccacgtttctg, tgaggttcagtaaccc, tacagtgcttgaaaga, aactaccccatatttt,
atcctatactttaact, aatcagctttttttgc, ggtttagcatagggaa, gtttaaatctgatccc,
tgagtctgacacggtg, gtcttgtgagatatct, ccaacatctatagctt, cttatgaaacgcaaaa,
ttttagggggggggtg, ccagcccccccaggct, tctgaccgatttaaga, aaggtgtctaattaac,
cacggagtctcgctta, cagccagaacatctta, gtaaaattgcataggg, agacactgaggaatcc,
gagtattaagactgga, caaagatctgtgtaaa, ccaagtttggatgagt, ctcgctgtcttcacca,
gaatctggctataatc, gcatctgagcctgttg, aggtatcaagccaggt, acatcttaactgtatg,
tgccttagccacccgt, ggaactgaggggatag, gggggggctaacagat, ggaccttcgggcagca,
catggtaatcacgttt, cccagattgatgttcc, cataatgccggcagtg, cgggtcccggtgtgca,
cttgttccctaggctt, gcctcttgctcaaatc, tccgtcccccccccga, tcaatctattcgtgca,
aaggtttaattgttaa, gtggtgccttacttac, ctcgtgcttaatgtga, ccctgactcctttcgc,
ataatcattaattggt, tcaggcaagggtttag, ctgttgataagtctat, atcatgtgacaatctt,
ctgtgtcagaaagacc, ttagtatcagtttccc, accatcatattgacca, tctttctacgtctgca,
gatagagtgcagcctg, ggggtcaagtgacctt, atgtaacagtcttaac, atgaccaacatggtta,
ctaaaaaagtgagtac, aagacctccccccgaa, gggtatatacatgggc, aaaggccatttatgta,
cggagtctcgcttatt, ctcggctccggctgct, ggctacagaagggttg, tactgggggtgttccc,
ccaacctatacatatc, gacttgtgtcaaactc, aatcattgctggcgca, gttccaccgtgcccgg,
ggcctaggataatctg, gtcctagttgcaagat, tgctccaaaatggagg, actggaagaaacgatt,
tgtcaaagtgagtatg, gctcagcaccaataga, agcccgttctctcact, tcagtgtttgtcgctc,
ctcatctgctttcttg, caggacgccgccccct, ctcggtgcctgtggct, aaaaagccataatcgt,
gactaatgaacgtgaa, gggtggctgctccgag, gatatgtccactaaac, ttggcgtcactgttct,
catgcggttgataaaa, ctctactcttattcat, ttttaacagctcggtt, gttttagggatccttc,
aacttctaacatgcaa, cttcgttgttttgttg, ttactaattagcagta, atgagcgacggagaag,
ttgatgacagggatgc, ccttttagtcagctgt, gctctatgggaaattt, accttaataggggcct,
tcgtctgtgaaaagtc, acaaaaaaaagccgtg, atacaggcacagctag, gtgtgacccgaggctt,
cgcatgagtttttttt, caattagctttactta, aaatgggggggggtac, tatcatccttcatatg,
ggagaactaatgctat, ataatcacaagttaac, gcctcctgatgacgtg, cttccacacagacggt,
ccgcagagcctctcca, cacactgaagcgtgcc, ctaggtccttcactgc, tacatcccaacaatga,
ttttcaacttatgtag, taatcgaccataaatg, tccagcgttcggcctg, gtaggtgacaaattag,
tctcattcctcgccgc, gggtttctttacacaa, ggtatcttctatccac, gcttggaggatatgac,
gctgaaacgccacaag, gcaccctccattgccc, ggaaaaaacacagacc, ctgatacatagctgta,
tgttcattaacagggc, caatactgggcttccg, aactggtaggcacccg, atcaatggtgagaggg,
gattccaatgggtgtg, ggagttgttgcactta, tgtggtcttgaatatg, tgacaattgtaatata,
tacattgctcatctca, aggtttagttacccac, cgcactgccccaaagt, ccggcgagttaataca,
cttgcggtgagcttag, gggtgcttaggagctt, caagttgtgtattcta, gtgcaaaaaaaaggtc,
taagctatctacctgc, ctcaagaaaggtatgt, agctattaggttgcaa, ttgaacgttgcagtaa,
agcgtagctcgaagga, ttggtcgttgttaatg, ccagtatcctaaatag, ggcatgtcatgggctg,
accctaagactaacaa, cccactttcgtggggg, ctattcgtgcacagtc, aaactgtggggggtag,
tcgtaatattataatg, taaagatcagttgtta, ctcaggcacttgccgc, atgccgggccggcctt,
agcgcaggaactgtga, gatcatctattagagg, tagaacaggaagatcg, agcacaatgtagtatc,
acctgtttatcccaaa, atatagaacaagctcc, ttcagctgattgggct, cgcttttagtttttcc,
ggaccacgtctccgtg, aatgtatgcaactcac, aatacctttgttgact, tccgccctcattcagg,
ctgacgctgctccatg, tggacctaccccctcc, ttggtcttcagcgatt, cgcacagtgccctcca,
acttatttttttcgtt, tggtgcaaacttgttt, tttgtaggttaatgct, gccaaaaaaaatcgat,
cgaaaaaaaggagaag, tttttttgactatacc, ggaaatacactatatc, tccggcaggcgccggc,
tagatggtccacgctc, tcagtttcgtcttatc, cttacttcaggtgaac, atcaaaaaaaactggt,
atgtagttttttagga, tacgcttttttttggc, caggactaggtctgac, ccgatagaacatccat,
aaagaagtctagtaaa, ggtaaaaagcttatca, ttagcattgaacacag, agtagactgtgcggtc,
tacgatgttggagcac, gtgaggtgctgcgcct, gaccgctgagtgtctt, ccggaggcccttccag,
catagacaaagaccta, aacagatagagctata, agcaaggtatttagtg, gggcaggctgaggata,
gaagcgtgccagaggg, gaaagatctcagactc, tgtcttacagattggc, gggtttgaccgcgtta,
tgttgaataacagcca, ttgtgaaccgccctcc, cgagggcggccttgcc, ttttaacgtttgtacc,
gctgcattgtaatctg, cttcacactccagtct, tccaaggggggggtca, ggaaggaaatacttgt,
gaacttacgtctgtct, gcgtgcatcttgcgcg, tgaaggtaaatgctcc, ccagtgcgtgttttct,
cttttaacccacagct, aactccccaaaggcta, ttacagacgttagaca, tgtacctccatttagg,
ggtggggtttgcagta, ggtttgcagcttagaa, cttcacgtgtgttcag, gcctcttatgagtggt,
gtaacaattctagcaa, tgagaggcataaggaa, gaggtccccatagagg, agcaaaaaaaaatcgt,
tctagattttccacaa, acacctgtgtaatgtt, gtccgcgcgagaggac, cttgtccccgagtcta,
actgagagcggccgcc, aactagagttagagtc, acattgctagggtggt, gcaccgtgctcgagct,
ccagagcattactgag, cacaggggggagctga, gcttactaaagttctt, cctataagggtgaacc,
catccggagtgagact, tgaactgattaagata, ttatgtgagaaagttc, gcttagtctcttgtta,
tatgatgacatcgcct, ataataatcaagtcca, ctaagcagactgccac, ataaaccataacactg,
acggcttctagcagct, aacagcttacccatac, gaacctatctagcctg, aaaatgtcggaaatat,
cgtgctcttgaaatcc, aaaaagtgttgacgtt, caactctacactgata, ggatgaggggggggtg,
actgtaggcctgctga, tccgcgcgagaggacc, cttggttttatatggg, ataactagtatcatgt,
ggtacttaagtgacct, aacggggggggaattt, atcgacaaggggcagg, cttaatatctgctccg,
tagactttgaatctta, agtaggatcaaagagg, gcggacttaaccccta, attagagagaaagcac,
aggaacagacatagat, ttggaagcttaagttg, gtttatgaatctgagc, ttgcccacattgtaga,
ttcgccttagctttcc, ggacagagccgcggcg, cttgggggccgggagg, aaagcggactgtacat,
tgagaacgttcagtgt, ctgaaaaaaaacggaa, acccacatagctgact, acgtgatggcaggact,
ttccttaaccctaaag, ctttgaggggatagaa, gagaagcataagttgg, gaccccggtagcgtga,
cccacaggcaacgtag, ctgcagttggtcggtg, ccttatggtagaacac, gatgtttcttgtatga,
acgaagatgattcttg, tttatccagtcatgat, aaacgaattgcagaaa, cggcacgatgtcggct,
agccatcggcaatgcc, caactttgggtgaccc, caagcgcccatttccc, acttatcggaggacag,
gaaacccaatgtcagg, acttcacggtgaagga, atgacttgcacaattg, tgagagaggatagcaa,
aatctgtagtactagt, gtccagctcttgttag, caagcaaaaaaagggg, agaggctcttagttgg,
tcaatagacaaagatc, catgatgtttagcatc, tgtctccttcgtatcc, gtggttgcagtgcttg,
catagaaagagttgta, ggcttagtgatggatg, tagtaaccaaccctct, agctgttggatcatat,
atctatccactactga, aagggactacacacat, tgctttccggggcatc, gtgcttatacatgctt,
tgggtcaactgagtgg, gacggcccgcgctagg, cgattggttacacccg, aacgggcatgcctgtt,
agactttagaaaagtg, aaccacctcttgaaaa, ttaccagtgtcatcta, acagagcacttagccc,
ggctaaggcatcagaa, tggattagttacctgt, caaccaggtgcagact, tgaaccccccccaaac,
accctgaagacaggtc, ttccgtgagcaaaact, aaaaaaacggggggct, ctcgtctgtgaaaagt,
gcttcggtgtggtttg, ttagcttaggatggcc, gttagtccattaaatc, acacttaataggctga,
cacggctggcccggcg, gcatctgcatgctgtc, ctaaaaaaaatcgata, tgtctaatgatgtggt,
tacatactgatcccac, tgtctctcagtgcagg, aaagggggggctgctt, accatcaggccccata,
gcattgcacaagtata, gctagtcccacctggg, attatctccaacgttt, tagcagcgattgtagg,
tccattgatgggattg, gagataatactcgagt, aacggactgtaaatca, gaatgattagaagaca,
cggccgggctctgtga, gtagacgctctgaatt, gggcggcggaccttcc, ggtaccctcattctgt,
gactgaagttttttta, gatctgcaaatgtaac, atttcgtttatgtcat, aatcccattaaattgt,
aacacgctgccaatgc, gtgacattaagcttag, ggataccatgctggac, tggggggggacacaac,
cagtattttgtaaggg, gcgtgaggagaaccgc, acacacactgaagcgt, caaatctattggttcc,
gtttggaattatcctg, tgcccgccacaagtat, ttgcatccccccacag, cccccgaactattttt,
atttctcaggaggata, gtgacatagttagact, attttcattgggttag, agaagagttaggacca,
gccgtgccggatccct, tatatgctcccctcct, gcctttcacatctgca, tctcctggcaccatac,
tctagtcatactagtt, agtgtgcagcactcaa, cggctcacggaaaact, atggaaaaaaacggga,
accaaatattgtaagg, cccattgtctgggtta, tgagggtccactgctt, tgccgcattgctccct,
cgtactttaattttgt, ttaatgcttttagtac, atagagctatattgtg, agacataagcagagag,
aaatgccaaagcgcct, aagaacttagctctaa, aaatggttctgagtag, aggcctaggcagaatc,
cagtatttggagttgg, catctcacctgcgtgt, cactcggataagacgc, gcatcgtgctgattcc,
tagaaaacggtaaaaa, ataagacaaactgggt, tggcgtttgtgtgcgt, agacccgcgggcgctt,
agcttaaccttccagc, agtgggggggtcttgc, gaccaacaatctgtgt, gactttgtggtgtcat,
gcaacagagtcggttt, gatcccaaaaaaacca, ccccgtctgtgacacc, aggcaagagctcactt,
ggtgaaagataagcaa, cccccaagggaggacc, ggccgggaagagtcgc, taactcggttaaaccc,
ttttcgtacacatttt, ccaggagttcagtctt, cggggtgcatcgtgct, tttgtagaggtaatga,
gacatccgccgctgtt, tacatccggagtgaga, ggcacaatcttgtctg, gggctggcagtttgcc,
aacaaggtgttgtagt, gttttcagggtatagt, cccccccaagggagga, gtcctgtgtataataa,
gtgcatgatattactg, atcattactcatgctg, cggcgcaccgtgagat, cacttgatcatggata,
cccttaggggcaagat, tttccccttctaagct, gcgttagccaccactt, gcccagcagctttagg,
cccgctaggcaccgcg, gcacagaactgcttct, attcatacgttatact, gcaaaaaagttagacc,
gcatctaggtgggtgc, ccagcataggaaagtc, ctaccaggtgctggtc, cgcgggtgcctggctg,
caataggtgaatacac, ggtggcagtcgccagt, gttgcgcacttccggg, ccaaaaaaagcgaaaa,
gtgttaaaaatagggt, tgtgctggtggtcgca, gtggggggtgtccctt, taattggttagtgctc,
taccaattggaagatg, cctatacttaaacatc, ccggaaactgtagtcc, catctaatgccattga,
gttagattagcaaggg, ccctccgagccaagta, ccaacctggttaaaca, tctaggtgatctgtgc,
gcatagtaacttccct, tataccgtctctggtc, ccgactgttggacggg, tgttaaacaagtacta,
gacgctccgccttaag, ttgtggattccaggga, tcaaaaggaggtcata, ataatttcgtattaac,
aggttcccccccttag, gcatcaagtaaactgt, acagcttatataaccc, gtctccgtggctgcgc,
acgactgaattaatat, gcactctggactctta, cttggatttgacctta, aatccccatcatccga,
caagatgaccagaccc, ccggcacttcccgtct, atatccgaaatatttt, catgttgctttgttag,
atagcgacttttttcc, tttcgattgaagaaaa, aggtacaggttatgtt, gtccctatttttttag,
atcgtggttttttttc, gtcttttttttgcgtg, ggcactcacacctcca, caagataactcttcaa,
cattgtatctcagttg, gctaggccgccgcact, cgtttgtggattggtg, tgaagctaagtagcta,
ccacccccctagccct, caaactcttaacctaa, tcgtattttgctctat, aaagtcccagcccccc,
ggaggccgcgtagaac, atgggttcctaaccta, ggtagagccgtggaga, tttgggatacgtacaa,
cttgggggggctacag, attgacctttgttcat, gtagtagtaagcagtg, ccaaaaaaacgccctt,
atctactgttcctcga, ggaaatagttaagctt, tctgtgcgcctgtctc, attttgtggggggggg,
aaccccttagaaaccg, tggggggggagctcca, ttacgtggcaaaaaat, cttgaaggtgtcacca,
tgactacagttaactc, aatcaatcaacgaaac, gtgcagctgggatcat, gcttcgacacggcttc,
ggaggtcttcccggca, gttatacacgtcccat, ctgcaatggggtgaac, agtgaaatagggaagc,
ttgtgggctttccatt, ggcttggtgtcatgcg, agcagaaaaatcttgt, atcccgatgtcaagag,
ggctaacagtcagtaa, tgcaaacagctgctaa, ctgattgcccccccag, tgttataccgtctctg,
ccagttactcgcctgt, tgctccactaagtaat, gtgttacctctgagtg, aacccctgcaatcaca,
tactccaatgacttgc, tcactagttgtctagt, tgtgcctcgtcctccc, ttttttagcccgataa,
tttccctcttatcgcc, aaaggacgcaagtgga, ttatctagtctgacca, agttctaactaacaca,
cactaacaggatctga, tcccaaaatgaacgta, ttgagtaaatcttcac, ggagacttgctatcat,
gagggggggggtgttg, gagggaaaccgtcttc, gtctaagcctttggca, ttctaagcaagtaaag,
gaatcctgccttgcag, tcaggcccccaagctg, attagggaggaacaaa, gccactcccgtctctg,
ccagacgcctttgcgg, aagcctccgccctggg, tccttgaagataacac, ctttacttggtcagct,
cctgaagagggggacc, ggggtaaggtagcctg, ggttagacccccgtct, atgtgtagaattcacc,
cttctcccaggttaag, tacacaaacccaatgg, cgtggcggccatgaaa, acattatggctcagca,
atgagaatcatatgtg, tcctcgctaacaggat, ctaatgactgcctttg, aacttcttattcaacc,
ggtattcgggaggctt, gttttacctatgtaca, agggtctgtgaagtcc, tctttatctagtggtc,
taaccgattagaatga, tttccatgtacgattt, gctgcgttgcactcca, ccccgataatttaatg,
gctactctgcatgagg, caggttaagtgggtca, gagctatagatactct, acttattgtatatggt,
ttcattgggtaagcat, ataaaaaaagaggacg, gacaggtattgttgtt, aaggggggggtctcaa,
taatgtcgcatctatt, gattagctcttatgag, accccgagaactatgc, agctagctgccctagt,
aatctccaaattccgt, cgggggggggagtggg, cttgataccttaaagt, aagttcgtaagatctg,
tgtgcactgtctcgga, acaatatctccccaga, cagcgtgtggcttaac, catctcctcgagttta,
gccggcgctaggcgct, ccggctatgccggcca, aaggtacaggttatgt, gcattgttcggatgat,
gtaaaaagttacacaa, cctcgtctcttggaat, aacccccccagcctat, ggctaggccctcagcc,
gcctggcgcggtgtcg, tgtagggagcatgctc, cgtttaaaaaaaacag, ggaacttttcccactg,
ttcccttaacaaaggt, ggtggtggcggggtag, gttgggatgcctgtct, ggacctcccctgtcat,
ggactggcatcggatc, aaagttagccactggc, cttaaagtctggaccc, ttttaccatgtgggcg,
aattttttcggtggaa, ggagtggatagtcagg, aggtatgaactggtct, accgtttttttggtaa,
gaggatagcattttga, gttaaatgaaatcgtg, cctgacactctgggtc, agactcgtcttgcggg,
cgctctgcctggtagc, caagtaaccacttatc, tgatctggtgagccac, ataaagaggggacact,
gctgctagataggctg, tgtagcttagagcggt, ataccactttcttgac, tataaaaaaagcctct,
ccagttctgtataatg, cagctcgggcgggggc, ccccttagctcattct, gcgagcaagaggtaga,
cgagcttagagaagaa, ctcgatctgatctctt, tctttcctaaccctgg, ggacatggctgaattt,
accacgtggaggtttc, gcttcctatgctcagg, gagccttgcccategg, ccctcccatcctatta,
aacacgaaggtggagg, acgacagactctggtg, cacgtacatatagaga, ggtcatgagtgcgaga,
cccgtcgccccgtcca, agcatgatgttgtata, taatctttttttcgga, tagggggggggacgca,
ctggggggtcaacttc, atagaacacgaaggtg, caaaggttttgcggaa, actgcttttttttagc,
ccgagccttgcgaggg, atctgacagagctaat, taacctcgaggtttta, aaatctagatagctaa,
atggcacgagcgcatc, cttagagcggttcctg, ctactttggaccaatt, ttactgactcaatctg,
ctcttcaaatgacctc, tgccatacagtgggca, gctataaattctatcc, agatgtgactttaggc,
cgagtctcctttattt, acaagggggggagaga, accacgtgcttctaat, aattcgacccagggtc,
atttttcgtcaccccc, gctggctcctttaact, gcagcgtctttgctag, aaaacagccccccccc,
ccacagtgctgtcgaa, atgagccagctacccc, cttcatacctcttgct, gtcttttgtggaacca,
gagccaatgctttacc, tatcattgggtagtct, agggttagaccccatc, gaggtgtgactctctc,
atgcggaatggggtaa, ttcgtgacatatttaa, agctcctttgactgca, attggagatgctggta,
tcgggttatgagtgca, agtttatcttaaagca, gtaaagactgcaggta, acaatacgcgttcatt,
ggactttcggtatgta, ctcaacctgcaaagtt, acccgatatgaaaatt, tcactaagatcccact,
tcagtctttgggaggg, taggtccttcactgcc, atacgtgggaagtgcc, gaggatgctgaccccc,
caacagggaggtttta, tctgagcaatcttctc, ggcagaggaccatgcg, ctttctccacgcaaca,
tccctcccaccagtaa, aattggttttttctaa, ttgctgagggttttcg, gcacatctggctcaag,
gaggcgctcgagtacg, tcccgagcatggagag, tcttgcgtcttcgtag, ctacgtgaaagtaaat,
attcagcggtgcggtt, gctcatacatagctta, gttcttgatctccatc, agaacgtgactagtgt,
gccctcagctgtaaag, gtttaaagcgttagag, cgctttttccccccta, cagataaacagatggg,
cccagtgtgcgctccc, ggagaggctcggggct, ttcctcgcgatacatc, gcataaaaaaaagtac,
catgtcactgtgctcg, tgcaatacagttggta, gaaaagcgtagcgagg, acgattttttttgttc,
aggagtgcatatacct, attttccgatttccct, atataactgtgagtcc, gttgttagggatctgg,
acgcagctagagccag, ccgcggccgtctcagg, ccccctcggacctgtt, aaaaatccccttaagg,
ataatacctaagagtt, ccctgccttataggca, agaacaaaacgtacag, tccactgtctgacact,
ggacatgggcggcacg, aacaattagttctatc, ctggcgaccctaaggc, agggctgctcacggtg,
ctgattggctctaatg, ttcacagtcactagtt, tctatcacctgatgac, cgcccatgcagtaact,
tcctgatgtatgacac, aaaccctgttacctgc, gattcaactgtttcac, aatgtggatggaacga,
gcattgcaggctttgc, cgtcccatacccccat, ttctatgtccacgcgt, tgtcaagctggagtgt,
tctagtatcttctact, gcatcgctaactaggc, ctcgctgatccaaaaa, caattcctgtcctgtt,
agacatgcccccccct, ggcatactgagtgttc, ttaccaagcatgcaga, ctccgcctcaaatgaa,
atagaccatatatctt, cgaggtaaatgactca, tcccttagagcaggac, tcaaaaaaaatgcggt,
tgtaatcattattagg, tttcgacatgttaaaa, ttgaaagaggggaaac, ttttgtaaagttatcg,
gcagcgtgtgtgtgcc, gcccgataggcggagg, tgtgggttcatttgga, gtacaaggagcattaa,
atcttgcgcgagccga, tgaagtaagatacatg, tggacttcctagactg, gtccttccgggggcgt,
atgtctccctgcctga, gtgctggttgaaatca, atgaacttttagggac, cactgtcagcgtaggg,
actgccattcgtggtg, ttctgttcatctcgaa, tctctgactctttagg, aggattgagccggctg,
gagcttatgtttatta, agatatgcaacatcct, agtctgcatgagtgga, ttgcaatcctcccgcc,
gtcctgaattataatg, ctgcagtgatcccaaa, ttcccgcattcatgct, aacactcacgtgatgg,
gaagaactcttaccga, ggcctgcccgaggcgc, ccacagcttatataac, agagaaccacaagacg,
caagctatcttcgtgc, tgcccggcacttcccg, ttagccacctttcctg, ccgcgaccccgccctg,
tacgccttcctttgtc, agggggggcaaaacag, agttcgttttgtaaat, agacggccatctctac,
tctcgggaaaaaattc, ccatccatgcccccga, tttacattgcgtctct, cattgtttcgcctttg,
cacattgctacttgag, ccactgcggcttagcg, cccgtcctcagaaaaa, tctgactgtaatactt,
tttgcactcggagagg, gctccactgctactgc, tcaacagtgatagtct, tttggggggtcagatg,
gggccaggcggtccgc, gtcggtctgcaaaact, gcccctgctgggaacg, tacccttccacctttg,
gcttatgctttgccat, gcaaatctctagtgct, caaatttcatgctcgt, tgtgattgtagtctaa,
gatgggatggaaatat, aagggctgcggacgct, tggcgttatcctggga, agaattcgagaaaaac,
cttgatttacagctct, aagtgattccttcacc, gtgtctaaaattgcca, acaatgcggatacctt,
ctctatgggcgtaggc, ttagccagtgcacgga, gtcaggttacttacac, gtattacacttcattt,
acctagcttccaccta, ccaaacgacaagtcca, ccaaaaaaaagatcgt, gccttactctgggtct,
gccaaaaaaaacctag, atgcctgaatctgcgt, atctaaacgcgctgcc, agcagggcaaggctcg,
taccactgacggtcac, tacgaaactgagtttt, gctctgaaagcctact, actccggggcaaaaaa,
ccctgacaccttagtg, agggtaatttatgcct, ataatctgttctcctc, acaattggcacctgtt,
agccttggttgctcag, aacccttttgacaccc, tgccccggtaccttag, tccactaattaggttg,
gtagtatattatagcc, actgtcagttccacgt, cttagtgttcaatggt, ccgcggcccttggctc,
actctatggggaacta, tagcatgggtgtgaga, aagcttttttttaggc, tcattggtggacatgc,
tgccgctcttcgaggt, atcctgaacattgaaa, gtcaacacaggaccca, aacgattttcctgtac,
ctaaccacagcactga, aacagaaaaaacggac, acgttgattgttaaat, catggggggggcagat,
ccaaaacgtaatgtta, aagatgggggggcact, taagttggtgcctgtc, tgtgacggcatcctct,
cagtcaggtctccacc, ggtacccctcacaaac, aatgactggactcgag, aaactgctgccgggaa,
gctcaggtcagggatc, tttagggaaacccttg, taatcttggatggctg, gcaccccccaacaaac,
ctgacgcttctgttct, gaataaggaagcttct, gggctaaaatcggaac, ctcaattgcgtacata,
tcaccggaaactgtag, cgcccctaaaccccat, cctaaaaagggaccag, ccactgtatcggtttt,
gaatatctattatcct, ctgcggcttagcgccg, ggagattccgtctgcg, atagacttgtactgag,
aatttttttcaaccgg, tatgggggggggcgag, ggaagtgtagtagtag, tttggaaccttgccca,
gcacggcaaaaaaaac, actgcagtgtcgcaat, gcgagttttggtgcag, atgcaacttgcctgat,
ttaattattttcccga, cgcgggctaagccctc, cccgtgtatcccgggg, ggagttgcatctgact,
gcttcaccctcactgg, tggtctgacgctgcta, cttcactccgacaagc, gatgcactaagggagg,
ccctgaaatggatcct, agaagaacctagatag, gacttgcgaggcctga, cgtgtcctctttactg,
ggagggggggttaaat, acagtggtatagccag, catcatacgcatctat, gttcagattgactctg,
atcgggttagaatggc, cttacctaactgcttg, ctgtggacgggatgga, tagcctcatgattcga,
cttctcaccacttgtc, tgggaacggatcctgg, actgccgggctctgag, ttcctttctttgccgg,
atactaattgctacag, aggagttgggttatat, tattttcaaggggggg, ctcgagtacgagcgaa,
ggccgtctcaggctct, atcaccaggcaatgta, ccctatgaggtttata, acttaatgggcaaaac,
cgccgataaaaatggt, cctctgcaatgaccta, ttcacgacagtaaaat, gccccccgccagcgca,
cctcatacatgtcttc, gtcacggccggggtag, gtgaccatacggtcaa, cacgtctgcatccatg,
acggcattttgacctt, agctttactccttcgt, ggcggctccagaccgt, aaaaaagagctcaacg,
tgtactacctttgcca, gtcagcttctcaggat, accactcctgtaaggg, gcgtgcccatttcccc,
ctttcttagctatagg, gaaggggaaacattcc, agcctctggtagccca, tctatgccacgaagtc,
atctcacatggggact, actccgataccaaaac, actacaattttccacc, ataggatttgaatctt,
tacattgcgtctctgc, gaagcagccaggcata, cccgcgaggcctaggg, tagaggcttgaaatgt,
gtccatgtcacaatgg, gattttgacttttgcc, cacatgatactgttac, cattaatgaggcaata,
atagttaaaccctatc, cgtttcctaaactttg, caagcttcttaaagct, ctcagcctaataatgg,
ccccaccgagccctca, gtagttttcgtaaaga, ttagctctttcacagt, cctccatagtgggagc,
ttgagaacgggggggg, agtacgttatatattt, gcagtgtacactattg, tattagtacgtttttg,
ccggggaaaaaaaata, aacgccgataaaaatg, gcttccaggaccactg, atacacatggaggttc,
ccccagaattcttacg, atgaggagcgtgaaaa, gagggtcatcaaccac, attgccctatcaaata,
aacgagagtatacata, ccttgcccatcgggaa, tggcttcacaactgct, actaacagatgaatat,
tggattattatctagg, ctgtgggcgggcactg, atagctaaaaaaaacg, gtttgtccaagattag,
ctaacttgattaatct, gagacctatgatgtag, ctgcgaggaccctgga, ctgcattgccggatga,
agtttagcccagatgc, tatagatccattcaga, atggttaaaccatgta, gataacatgctattag,
aatgactatactggct, aaggaggacaaattag, aatatctcctataccg, agagcacatgtcagct,
ctacttcccagggtgg, tcttgcgcgagccgaa, gtaggagggtggtcac, gcttaatctgttacac,
gccttgcctagctccc, ttctctgctggtcctc, ctggtaaaactatcag, ctgtactaagttgtat,
agcacactagtacact, ggggctctccccgcca, ggaggcagatcaggac, agttaacccccccatg,
ctttaatgcccccctt, taatactctgtcgtac, aacctatgctagggaa, ccattagaaccttcca,
aaaatcgtgtagaacg, agtcttgagctattac, agggacggcactcaca, actgaccagttaccaa,
ttcagccacctccaag, agaggataatctgctc, gctgctcgcagcgctt, tgcaccgtccccccta,
ttctgagtagaacccc, acgttgactttttttc, gatagggttaaatggt, cccccgaaaaaaagat,
ctttgacataaagggt, tctatgggaaacttcc, tacttatctcagtact, ccctcttaggacaccg,
gctgattgggctaggc, cattttttaggggagt, gtattgcttcgctgag, tgagaaaaaaatagcg,
ttaatcatgggagtgc, tgattagtcccaagtt, actcggagaggccgcc, tacactggaatgttaa,
ggcacggctggcccgg, tgacttgccattatca, gggatatttaacttaa, ccccgtttttttattg,
ggcattatggtcttcg, cttagtctcctaattg, tttcccggagctcctc, cctgcgggtttccatt,
ctactcattaatgtcg, tcagactctcggttaa, cagtatcctaaataga, agcagtgcactatatt,
acgctgttatttcagc, ctcaaaaatgatacgc, ctcccatcatgccacg, catgctcattttgatt,
tctgaggctcgcaccc, cctttctaggtaagga, agattcgtcacaatta, ccaggtagtcctgaag,
ccgagagaaatatata, tggtggatcctaagag, cgacatcagcggttca, aggaccccctaagggg,
aactcgggaagcttag, gcgcctcccgctatgc, tatcttaggaccaata, ggcggttttttttggt,
tcaacccactggattc, ttcccattaggctacc, ctcttatggtccttgc, acagggcctataaatg,
acaacagagttaccaa, gagtattacccatatg, agctcggtcacacatg, actatgtggccaggtt,
aggtgccataagcaga, atgtaccccccccaat, ttccacctcatcgtca, atagagttggcactgt,
tgtacggtacaccacg, agaacaggatagttgg, gtgccgcacaccccag, tgatgttttttttagt,
cccatgattgaactac, actatcagacagcaac, tgtgaaattaccaaac, tattatagccctaaag,
ccgcggggtactctgc, tgccccagcctactaa, ttacacagacaacctc, ggatacatttggatct,
tagagtttgtttgggt, gagcccatggacattc, ggatacgtgggaagtg, atttagaatgggccat,
cagtcttgtcatgtat, agcagttctaatcctg, tatattgcatgggagg, agccccccccaagggc,
cctgtgaggggggggg, catgataacagaatgg, agcttttgtatcagtg, aggggggcaacagcat,
tacccacgatgaacga, cttggtatctagggag, cattagaatcgctttt, ttaaccacgttgtcca,
cttgtgttcctgcagg, tcaatgtccgtgagct, agggtcgggggccgtg, caagatggacacttaa,
ggcctttagatgtatg, ttcgaaaaaaagagaa, ttgggtaatatctacc, gccgtccgcccggagc,
gaaaatccctgtctta, cgcatcccgctctgtg, tctctgaactacgtta, ctttatctcggaattc,
gtcttgggcatttcca, tccctgatcaagtccg, actctgagtgaagagc, caagaccctttccatt,
aaataataaacgcagg, agtgaaatatcgtcaa, catactttcatatgac, cacggagttttttttg,
ttcattacccttctga, aacttgtatgtctcac, atggggggggcagata, gcaatcaggccattac,
tgtaacgcaatttaaa, ttcaaactattcggtt, caattagaccaagcct, attcactgacactagg,
cagaggaggttgcgaa, gcttcactccgacaag, tgggcagcaggagtta, agtcccccacttatgc,
taatacaccagttcac, ctttgataccccagag, cacagegcccgcctta, ccacctcgctgccctt,
ataaatggattgcgtt, caaatcgtatcttgtc, gtcctaaaaaaacacg, gacgtctggaactggg,
ttgagccctcttgttt, gagttgcaaaaagaac, ggcaagatctgcctct, actcgcgccactactg,
cagtcatgaaccccct, aggtgtacggtacacc, gactggggtccctctc, tttttacttactaacg,
tcattgcggctggaca, tctgaaggcatgtgtc, cctaacaagttcgtaa, acattccacatatcac,
aaacgttagcactaca, tctcgttgggaaatga, ggcacttaagtggtcc, agaattgtccccctca,
gttttccttgactagt, ggttacacacgcctgt, ccaacagcttacaagc, ctgtcactatagggtg,
ctcgacgccttttttt, gagattccgtctgcga, cctgctgcatataact, acgtctccgtggctgc,
gcggaccttcctctcc, agaatctagtctatta, gcgtgaagcctgcccg, tgaaaaaaaggggtca,
cgtacaaaaaaaaggg, gaagtcggattaagca, gtaggttttttttagc, gaggggggggactaag,
aaagcttccgcactct, gtgtcacaatagaggt, cactggggaggacgaa, ctcacagaggactgaa,
caagccgctgtgtgga, tagaatccttgagtag, aatttgtttttttgcg, tgagagcggccgccga,
tctccgactgttggac, cctccgcctcattggt, aagacacccaacgagg, atttaagtcccatttt,
cccaactggtgagtat, tagggagtggtgatta, tcaagtccggcaggcg, cactcaacatttgcgt,
tcacgctggaaccaca, ccttcccctgtttaat, ttggcccctaagataa, gcgtttttttagagac,
gcattttagatcaagg, atgctttctccgtgta, atctttatctagtggt, cctaggtggtggggtc,
aaaaaaacggggggta, aaaaaaacggggggtt, gtaaccgcatatgtta, aaggaccagactcact,
cagttagacttgcagc, acagagatcaattcat, ttgctactatggttca, tggttcctgtctcggc,
tgcttcactegcccca, tcctctatagtccacg, acgagagtatacatac, aatagggggaggctca,
ggcccgttactgaggt, accaggtgttcaatga, cccctattaatcgtcc, tttaggggggggtggg,
gaggtgattccaccaa, ccctggtgtggttgac, tacaagactgtccaat, gtgacccgagcacttt,
ccctcacgtcaggaaa, tcatcaattaagcatg, gcggtgcggttggttt, cattccggacgggcat,
cgaggtacctcggggg, agatggcgttgttgta, tccccgagccttgcga, actcatgactccctct,
agtgggcaaagggcgt, tcacaatgagtatgct, cttggggggggttgta, acacttttgttccatg,
tgcctcgggttgtata, cccggctaaaacggtt, ggccctcaaaaaaaat, cacttgtattccccta,
gttaagagcttttttg, catccccccccgaaaa, taggataggagttatt, cgaatacaaaaaaaac,
tgatcattcactctgc, ctcagctggtacctta, tgtaggtgttaggatt, aagaggcgtattttac,
ttcttgctgtctagta, cctcacggtagtccag, ggtaaaaaaaataacg, gtcccccctagtgaat,
acacagggagacggcc, cgggcgcactattgct, ctgtgccattaggaat, ggcaaccatccggtga,
atggagagctaaagtt, agcatcatacagatac, tcagagtaactctagg, tgttacccgcacatct,
gttcagtgtgcttgac, tctggaccattctgag, tcttatgtgtccctaa, ctaacatactatctat,
aataatcactggccgt, cgtgagcatacaggag, aagctcggtcacacat, tcagtcacaaatcatt,
tacagttgcatgcctg, accctttgctgatccg, cacatgtttttttgga, aaaaactcgtgttatt,
tggcatactgactctt, aacgcacttcacaggt, ggtgagcttagatcgt, gaccttcaagggagac,
tggccggagtagggac, agtatgtggtgttatg, gctctcacacggtgcg, agcttcaaatgactgt,
cgtaactgttcagcct, tgagcaacctccttcg, ttctgcattaacagac, ccaggactaagtaagg,
atgtatgcaactcact, tattaaaaaaggtagc, tcataagctggatatt, tctgatgcccgcttga,
agcaattttgcatcag, aacttcccatgtagta, ctacctcgggaggctt, ttcgggtcccggtgtg,
ttagatcttatgtaag, gacctaagaccataga, tgaggaggttcagacc, agagcttgcggacaac,
aatgtatacaaatcgt, cgcaaactaacaatct, agaacccctttgaagt, ccctacctgtaaacac,
agatatactcttacct, tttattaggccagctg, tccactcggataagac, cagtattgggggggtg,
ttaccacaatcaataa, ttctgggctttaagtt, caacgttctcatcttc, cggtaccttagcctgg,
agcatagctatcagca, cctgtccaacttggag, gtgtaatatcccgaat, gcgttatgcctgagcc,
acgatgtaggagtcag, tcctctggttggaccc, aaaccttaaatagtgt, gtgggtgcccggcgct,
ctaacgcattgttcgg, gatttcatgcattcac, gccactgtatcggttt, agatgttcaagcagga,
gttttttttacgcaaa, ccctatgtggtactat, cacagttagacaccct, tgccgggtttttttta,
gggtctcggcctggca, acatatcaatgcattg, tcagtcgtccaagtag, aattgaaatactgcca,
gcaaggcaaccttctc, tactggacggggcggc, gaatttttttttagcg, gtcgtatatatatctt,
tcggcttagaccccaa, tttcggtggaagagga, ttggattgtacaattt, agatactttgcccctt,
gatccatctaggccgg, cttcatgtagttcctt, ttaacgtcacgctttt, acttgtttcgagaatt,
tgctatgctctcattc, ccttatgttggccgag, agggcggttttattat, atcgccaccagtatgc,
cgaatgaacccctgaa, aaaagactgtctatta, agcggtgcggttggtt, ctcaccacgaaggtat,
ctacagagatctttaa, tctttgccactcctga, agtcccggcccccatc, cagcccccccctcctt,
ccactcccgtctctgt, aatctaaacactagag, tggtatgttgggtgcc, tctatacaatgtagtt,
tatgttacgttttata, agatgcctactctcgt, aattatctatacgctt, aacttaatggacccct,
atagcagcgattgtag, gtcatatgacccctta, tggctctggctgaagt, atattcatggggggga,
catcccgagcatggag, tgaagaaggagaacgc, ggccggataaggaagt, cggaacgtgggggtct,
gcgtggtgacgctcgc, ctcctgtcaatcctgg, acctatagacgtatag, ggggaatgatgtcccg,
taggcgttttttttaa, aaatttgcgcagaaat, tccattgcttatactc, ggggcgggtctccgac,
aggacttgctgttgtt, ggcgctctgatctaaa, cagcgcggggcccttg, cgatctcctgatctaa,
gctggggggacccgag, accattagcaggatgt, aaaaggttactagttt, gctggccctcagctgt,
ttatggtaaaaaactg, atagcttttttaggcc, cattagagccccgaac, tttaaccacacttata,
tgccgttaccagcatg, caaacgttttgtccgg, attagtcgtttcatcc, ccctccttcgtgcctt,
gataatcactatgtgg, actgatgcttgcgagc, cgggataaaactaagt, atgtggctcactacat,
cactttcgtggggggt, agacaagataagagcc, ttaacgcatgcaaagc, agtgctgcgttgcgca,
ctttatcatcactttc, tcataggtcagaactt, ccacagacagcacgtg, cagaggagttccaaca,
toccaagcgagggagg, acggtatcaccagggg, gagcccctgactcttg, tgggaacgaggggtct,
gctggcaccacatcta, tcaccccacgcataat, ttcccagcagccacgc, tgttttggtagcgttg,
ggatagttttagtgcc, agattataacacgaat, taatgtacccacctcg, ttggatttgaccttat,
catcccaatatgctca, tgcaaacacattaggc, aagtctgtaataaacc, aggcaaccttctcgtt,
gcaccctgctggcatt, tgccggcagtgaccag, ttcctattgcctgcgc, gtcctttcgctctgtt,
tgagatgaactaaggt, atcagccggatgtggg, tatgtttttttagagc, actgatgcatcatggc,
actctgcccgatcgcc, atgctgtacacattcc, ctgtactacaggcact, tagggtgatactaaat,
ccgtgttgtgcagctg, tcacgaaatgcagtgg, actcctaaccctgaga, tcttccaaacccatct,
tcgaaatttttttgca, ctacttccgcagaatc, tcgacagccttaatgt, gcctaaaaaaagtatc,
ctgagcttattcaatt, acaaggtgttgtagtc, agttaacttatcagca, cctatagttccactct,
ctgttaaaatcccccc, ccttaatagctgctca, acttaagctacaccta, acttgtaaccaagttg,
acgttaagtcctccct, gttttcgggtttggtt, atgggcgtaggccccc, gtcctaagcatgactg,
tggaccgatgccaggc, tacctctccttgcagc, ctgtcactccttacct, ggagtcagtcctttga,
caggatcttaagtggg, gggcgctcttagtccc, aaccataatggctcgt, aatccaggtgtatgca,
aacctaagtgtacgtc, gcccaacctccgcatg, ggcctgcgtacagcag, catcaagtaaactgtg,
catcacatgtttgtat, cgggattcccatactg, gaaatcaatacttgta, caaggcttctatctga,
gactgtggacgggatg, gcatggtgcgagggac, tggcatgggtattaac, acttgccgcagtcagg,
acatgcctaaataact, cggaagggaacagctg, ttccggtctgtcattc, tggacagtacggtgat,
ttagagattttcgtca, tactggtaaaaaaact, agtcaggattgtagca, agcccggctatgccgg,
aggtaaaaaacaacta, ctggagccgggagcgc, aacttaaagtgaacga, cggctgtggtgctacc,
ataattccgagtcaga, ttattttacagtaccc, accctaggtgcctatc, gcaggggatgtctccc,
cacccttttagatggc, taaatggctaaaccct, ccttgttgtctgtata, cggttcacttgggctg,
ctccgtgttgtgcagc, tccgtcagcgtgtgga, atacaaatcgtcacag, gtgtccagcaaaattt,
tttagggggggttaat, taagtgtctaattatt, tttagtaaccaatcac, aagggatccaggcccg,
ggagggaaaaaaacgt, ttttgagcttgtgagg, gaaagtctaccacttt, gcttatgcccccacct,
ccccccgcacacacag, ttgtttgtctaacctc, atacttttttttgggc, gaggttcaaatagtct,
gaatacttcccccctc, agtcattctaagcagc, cagatagagttctaga, ctccgatgaataagaa,
agatatagtgaagtac, catgacttaaagtatc, cctgatagcatcctct, gaagacaataggtgga,
ggcctacgaaagtgtt, gtaatctcatccctat, cctaagggggtgcagg, ggtagcctttacagca,
gcccacagtgctgtcg, ataagcatatagcacc, gccagcacacattggg, ccgtgtgacttcagcc,
gatcaggatgaccata, tctgtcctacccggcc, gaaggccctaacttcc, attacagtgcgcccca,
gcgtaaaaaaaatgta, tacctttaacctcaac, gacctaagtcacattt, agattgcacctctgag,
gcgatgctcagggcaa, taggcctaaaaaaatc, ttcgcaacaagttttt, cgcaagccaagcacct,
gtcatctctgtcatga, aatataaggctccagt, ctgagagcggccgccg, agtgcaagacaaatga,
tgacgtgtgcatgcag, accacgttgcccagag, ccatacagtgggcaca, ttccacacagacggtc,
gagctgcacactgaac, tgcccgtgccttctta, caacgegcccccccca, tgccaccgctcttctt,
taatggagagcgtctt, ttccacgtgaaagata, acaccatgaaacttag, attcggagatataccc,
gttagaagacagccac, cccatgtcgcccaggt, ttgaaagtgaacacag, cattaattagtttgtg,
gacaccggggatgacc, ttaaatagtctgctgt, cggttctcttgtctta, cagaggttccttgatt,
agtatcagtgtcctaa, ttagcttttggagcct, agtacccgtataggta, agccctgaaataacgc,
cactaactaaaatgac, caagagcggtgagtca, ccgccactagatggcg, agactgtgcggtctgg,
gagttagaacacgcgg, atatcataaacatcct, cctaatgaatggatgg, actgtacaaatagtta,
catatgaacccattac, aacctcaaattaacgt, cagtaggcagccagat, aattcgctaaaataag,
acgtgattaactttac, tgacagtcactcagag, atgcttgcgagcatag, taagtcttatgttaag,
cttatcctccctattt, ccaaaggacattagcc, ctctccaatgtgctga, cccagctattttgctg,
aggaagtaaccgcagt, gacccacacgggacca, gcgtgttctcttgagg, gagttgcacccgtgca,
aaggggggggatgtgg, ttatcatctgcttagc, acgtgtagaaaccctg, ggtggcacattttggc,
cttatagaatatccac, cagtggatgacttggc, acacgtatttctaatt, atagtcaagacgcaga,
ccttcactcaaaccaa, gcggagctaggctccg, tggagaaaacgggctt, gcttgcagctcagatt,
catgccacgtgctctc, agacgccctgtccggg, gtcctactaggtgaag, gcgaaaagaatgacag,
agtttatgaccctaca, aggaggataggcagta, atctgagagagactta, ttgagacaagactccc,
ggaatgcacgccgtag, gggcgggttgggaatg, gtcgttatgtggaaag, aggttcaaatagtctt,
accacttcctttctca, aatgtacctaacaagc, acgggtgtgcctaagg, tgtgtgttaagatcga,
cgagaaaaaggcgaag, ggtaggctgaggtgta, aacactccgtctaaca, aaacctgggaacacta,
agcgtcttcttactaa, atatgggttgagagtc, tatacttcgtatacat, ctagagcagtcccatc,
cttgcgagggcagccg, attgtcatcacgtttt, gcttaagtgcagttgc, gttcccggcgggtgca,
tatgggggggtgtcta, gcaggggacagcatat, ctcatagactactaaa, cccacatctgttcaaa,
ggaagagcttggcccg, ttaagggcatttgagc, tccagtttgggccgga, cttatatggctgttga,
acaagcctcttttttg, attttttaggggggta, ccccaatctagcaact, agctagggggggagca,
tatatacatggggggg, gccccggtaccttagc, gttttttcatgccttc, ccaccttcaataatgt,
ttcacaaaccttactt, ggcgcgggctaagccc, tcctacccctctggtt, gacacactagagcatc,
cttgttgtataggttt, ccgatgatgcagaaag, gtgcgggtcctgattg, aggcctggaaaactcg,
gattgactctgagact, ttcctttagtgcaacc, aaccccccttccctac, ctaggtcatctgatag,
gtgagtgagttcgatg, ggcctaagtttttttg, catgtgcaccatcaaa, ggtcaataattgttcc,
cccaagtagggcaaaa, caccgtaccagacttc, aacaaagggattgtgt, ctcccttaagatgatt,
ccgcatgcagctgacg, atcagcgtcaatgcca, ggagctgacagtctat, tgcttttttttaatcg,
ggggaggggagggcat, aagatgaccatgctgt, taggagggtggtcaca, gcttcaatgctaagta,
acttacagttagtaca, ccagtttttttatcac, ctatctcttttcgcat, gagctagtgaaatctc,
atcgtcacagcagcat, tggtattttttttcgg, ctagtttcccttgtta, gagggctttataaagt,
gatccttgtttactgc, tttatagaagatgtca, caagccatgccccgtg, tgtcgccgctcagcat,
ttctcatactagtgca, atgattttcattgatc, atggttctgagtagaa, cggtgttccctttgga,
cccctgctcttatcct, ctagagattcttaatg, cacgaagaagaagatg, cacactggggggggtg,
agataagtagtttgtg, gcattctccaccctgc, catgtcaaagagggac, gcaggacatctcccca,
gatatggggggggcac, gccaaggtagctccat, cttgcttctgatggga, cttttaatggagagcg,
cgctcgcggccttgga, atcacttaacacacca, aatcgtggcataaggg, gatgaactggtgtaat,
gccggccttgctttac, tttgcgacagggtgtc, ctccgactccggggca, aagataaaaaaagcgg,
tgctttttgtacaacg, accgaaaaaatggtga, actgttcctcacctag, cctatgggacagggtg,
taactcccttttacac, aggggcctcgtcaggc, ctactggtgcacttta, aggccggcgagttaat,
taacaatgcattagac, tggacttaacttaaaa, atagtaggggctccac, agaaactagggaacgg,
gtaacatattatactg, tacatccccatctgag, cctctgaggttgcagt, ctctgcgatgcatctt,
gaatgcatatacttct, gttctttttttatagg, taaaatatggtggatc, aaaactcgtaagcact,
cacttgggtggtcaga, ctcgtccctggcctct, gtgaccccgcaaggag, accggagcagcctgag,
aacgaccgatgaaagg, ttctacattttggcga, acgaaaaaaaacagga, ctcctcgtgaccaagt,
gtgtgttacggggcag, gagagttccagtggtg, gcgttactctgtctca, gtcacgttcttaggta,
ttcgtgagagcagcag, gatcgacccacctggg, gaggtttattaaacta, tgcttagaatagcctg,
ttacctttgaggatcg, gatcctgtacctacat, ctccccccccgggtat, gttatagaaagctaaa,
ggtggtcgcatgaatc, tattgaatgcagcgtt, ccaggcctggtatatc, acccatcaagatgggg,
agcaaccacaaaaggt, aggacttaacttactg, accttgcccggctctt, acctcgtttgtactaa,
ctaaaaaaaggcgggt, tctcttatctcatatc, tgttatccaataggtg, tagaagtagtctcgct,
aggacctatggtccca, aatataagcctttagc, accacgcctgttatcc, tgttttaacctgatgc,
accttttttgctcaca, ttctctttcacgctga, agcggcacgatgtcgg, gctcggaggcacaaga,
tcgtgggaaaaaaatc, caaagcgagcaagagg, gttgtatagtactgcg, ccacagtacgtggttc,
cacaatgtgcgctagc, cacttaaaacttcacg, cttccagcttagtcag, tcatctggaaccgaaa,
atacgaacttaataaa, gatttgatctccaaaa, tctgtatgtctaggag, tcatgtcagcctaaac,
accacgtctgtaataa, atgtaaaactccggtt, agccctagctggaaac, cgcccatggcattgct,
tttggcccaaagttgg, tactgacttgccagac, ctccctgtaattactt, tatatgccttactcac,
acggtacaccacggga, acattgtcacccccac, gtgcgccgcccccccc, gcttacaaaaaaaagg,
gtgaatgtaaacgtga, gctttgggtggcttag, ttacaatctcccctaa, tctacataacctggat,
tcgggattacatgcgt, gtcaattagttggaaa, cgctgagaaaagtcgt, gtgaggcactatgcac,
tttgcattaagaccct, taccacttgcatgtct, tacaatgtgtccagtt, agtgtctccgccccat,
tacacttaccataata, tatttctgccagctca, gtgaaccactttagca, acctgtgccgcgtcct,
gcaggtcaaggcctat, acatttgacagtaggg, atggagataagatcca, ccattatctccaacgt,
gatctctctgagatgc, aggacagagccgcggc, aagaacttctatcata, ccctctcccttgatca,
attgccccacctgttt, aagcagagctcgtctc, gtgtcctctatgaatg, aggacaagcacctgat,
cccaggacgctcccat, ctatgtgcaatagtaa, gcctctaagtcagagc, tccatctatgccacga,
acgaaagtgagtatat, actcccatagcactcc, ctggagatatccagta, ggttctagaaatctgg,
atatgcagaacaaacc, gtttgtctattggtat, gagtcccgaggcactg, cctacatatttaaatc,
tagccgtaaatcactt, ccacgtctccgtggct, agatcacgaccccccc, aggcttagccctatat,
gottccccccctcttt, aagacaaaaaaagcgt, aaacccggtacctgag, tggaaacatcgcaaaa,
gtgtgcgagaaggcag, gtactttgtttaacat, atgttagatacttcct, atggagtcttactcat,
tcttactcaggcaacc, tagtgaagggggctgt, acgccgataaaaatgg, cgatagggccacttca,
ggccaattgacataca, atgggtacaacccaca, ccgcgggacacatgtg, cgttttttttctatat,
ctccatctcggtttcg, ccaaatttcgttcaga, ccataggaggatgttc, tagagtcaccaccacg,
gaggtagttggagtaa, ttcagcggtgcggttg, atgcaactgtaacatg, cccagtccccccccca,
gagttagggtgcaccc, tgtcattgtgactcat, tggtgggatgctctgc, cggcattttttttaaa,
agtcagatttaggctc, gtattgagtcatcccc, ttaaatgaagtctagc, tttaacgtgccggttt,
tagtgctactttaact, gccggccactgaagca, ttacaggaacctttgg, gcgtcaatgccaccgc,
tatgagataaggatgt, tcgtttcagttgtttt, ttactgggatacatat, acgtttttgtactgat,
tattgccagtctgagc, tgttacctagctacaa, ctttccttaagggtgt, cactcaagcccgtggg,
tccgcggccgtctcag, tggtcttagagaccat, agcttcactccgacaa, ttcctttgtgcgccgg,
aacttcctctatccat, ttttacaggcttggtc, tatcgaccccctgccc, tacctatagacgtata,
gttgctgaatgaacta, ttagatctgggtgggg, cacaatcaggggcaca, ttcaatcgctaaaaat,
gccaattgcagaaacg, gatgccgggccggcct, tatcacagcatcgctt, tactgagcctgaaaga,
cgctatgcagctcaca, aaaggcacttcttgta, acccagttgaaaagta, gaatgtcgtttttttg,
cttctgacaggggaga, taaccgtttttttggt, cgaaatttttttgcat, atcttgacactctatc,
ggaaaggggggggaaa, gttgaacttataggaa, cgaggccgtagggtca, attgtgagacccaaca,
gattcctaagtcccgg, acagtctcagtgattc, tgtggtctagattgtt, tccttcaggtgataag,
gaaatttgcgcagaaa, tgaatatctaatatgg, ccacctttcatagggt, aggaagtatcctgcta,
acgccccacatgtcgc, cttagagacaggcttc, ccgctgtgcatcctct, gagtgcttaaagataa,
gatactggaaccagaa, gacagattatggcgtc, ctggtgaagctttacc, cccgagcatctggaac,
aaagtagcccagtcag, cttctcaggcagtatg, ctgggcaggggggggt, tctatagtccacgaga,
atgttgagagagtgac, aacgggcacacgacag, gggtcccaaggtactt, ttaatctttcaccagt,
ttcctaagtcccggat, tacggcatcaaaaaaa, ttcccttagttataat, agacagataatagttg,
tcgtcttggttttttg, cacattgttctgtcag, acggatgacctcgtgg, ttcagctatcattatg,
tgactgctatggatta, ttcctccgacagatta, gccatactcacctatt, ggccttgcgaccgccc,
tggagttgcgatggtg, gttcagcgcattcaga, tgggtagccttactct, tgctatagaggcagac,
ggtaccacgtgactct, tcaaaaaaagcgaata, tttgagacggtctgac, tcccttcttaagtttc,
gggagcagccgcaggt, ccaacctttggtaagc, aaccactttactcacc, tatgtttcatagtagt,
cgatcctcgcccctca, agcctgagaattaata, aagcaccttttttagc, tgggggggggctgctt,
gagtctaaaaaaagcc, aatggttaccgcaaac, catgaggtctgagttc, agctagtctacttatg,
gctttccggggcatca, tgtgacccccccccgc, tcactccaggactgtc, ctgaactacctgtatt,
agcgtgtggatgggct, aatgctgggatagact, caaactgctgcaacct, ttttcttgggctaacg,
cctaggcttttgtctg, agtactaagaattaaa, tcctttagcaggatgt, gtaaagatcatgaagg,
aatgtaaaactccggt, ctctgtggtcacgaat, cagtccccactttcgt, ctaacttacctaccat,
acctgtgtcagggtgc, caggtgtcgcttaaaa, tcgcaaagtgtcaggt, tctgtactgactctaa,
ggaggagtagctacca, catatgttgtcgttgt, aaaaaaacatgcgagc, tctgagctcgttgcaa,
gggaggccacaccgcc, ttaactaccagtgtga, tctgctgaattgggcc, tccccgctaggcaccg,
aagcatgaataatctc, cagcaactctaagggc, tattgaagtcctctcc, ataaccttcacgatct,
atagatccccctccct, ctccaaaaatcaacga, tcagttcaacaggcca, gccaaccctgtaacca,
gggccagcgtcggccc, tcatgactatagcata, gatgcggcagcccctt, tactagcagttctaat,
ttgacccacctttcat, gatcacaggtctattc, tccagctacgtgtgag, agagtcttcgggtgac,
acacagtttgcgatat, cgatacgcgagcccaa, tcctgcgtagagatat, ctgagaggccgccgga,
tgtccactcctgtatg, aagagcaatgatgtaa, gcgtcacagaggctgg, tagcaactcaggccaa,
gcatagctgatccctc, gcttaacaacatattc, ccgcgcccgctcagcg, gcggagaaccttgttc,
gggaatttcgtggcct, ctctgaagcctcgtgc, tgatctcttcgttctt, gtccattagagcaacc,
ctctgggactgtatgc, ctggtgattcgatttt, aaatgccccggtacct, agacaggagccggtct,
cttgtcgaccaggttg, cgtccggtgctgacga, gcctgcgccccccctt, gcgtgaaatccttcca,
aggggccttgcattca, tttacaatgcggatac, tcaagaaggccaactc, tggacaaggttcctaa,
ggactcgagaggtcca, aggactttcggtatgt, gacacgtgcacatacc, gaccatgcaggatcgt,
ttaacacttctgtagg, cccctaagggggcaga, agcgcgcggggactcg, ttgaatgcgttttgtt,
gtggagggggggcaag, catttagactaagtat, gtaacgattttttttc, tgacttagaaaggatc,
aggtggtgctttctcc, tccctaggaaaaaacg, attcgtttgtacttat, caacttccctttagtg,
ccactacaggcagegg, ctactaatcctgaaac, atggcctctgcagtgt, ctcctgctcgcaccct,
aatgtcgcaaccttcc, ggtgtaacatccagga, gcctttgaagatctat, atctggcctgccggct,
accagaggagtatact, ggttctggcactcact, gcgagagcatcatttt, gtaaatgttgctgccc,
aaaatgggggggaaga, tgcatgtcaagcaaaa, caggcgggcttagtcc, aagccgagtttggctg,
gagctctcgtcttccc, gctttctccgtgtagg, tttaggggggggagag, ggacacattctctagt,
gtgcttttgccggctt, gtcgacagccttaatg, ttgcagctgcggtcag, cttccaattatttagt,
ccttgctccaatgaag, gtgtaccctatatatt, accacaactctattat, tgtacccacctcggtc,
agcggagcttgtggtt, taaaaaaaaacgattt, taactttcccatgagg, gctccaaaaaaaatgg,
gccaccgtggtggctg, ccactcggataagacg, catggtaggcctataa, atcccccccatttggg,
ggcctggtgaacattc, tcgaggtctcgccact, ggtctgggaagcctcg, tgaggggggagcggtt,
actcacactgcttaat, gatatctgattgcagt, gaaattccgtctacaa, ccacaatgcgccagtt,
agcgagaaaaaggcga, aggtgatcctgctgac, ccacgaaaaaaacccc, ctttcgcatcctgaga,
tccggttctgacaggt, agtgtaagcaaatgta, atggactacctgaccc, ttagctggttctataa,
gttagacaccgcgcct, cattgcggctggacat, atggaggcttgtacca, gttactaccagtatct,
tcaagcaaggaacggc, aaacgttactaatgga, accttagactgttgtt, atgacctgcagcacaa,
gctgttattaaaactc, cagatgatgccggagc, tccgtgttgttccctg, ctgtgttactaatcgt,
agtgatgcaatgatag, cctggatgctgaagta, gacttgctgacggaag, atcggtgtttttttta,
tacaatccgaaataga, acagtgtaaggtaagt, cccaatttgtatcaca, cttgcgtgttctcttg,
aaattattggtccacc, atgggccaagttctga, tttggtagtggaaatc, aggccctgccaaatgt,
aagagccctcggttgg, atctatccttactgtc, atcccccccccattag, gggatcaattttttat,
tttgcagagttggttg, gcgtgggcaacaatag, ccgcgttttcaccgga, cagcgtctttgctagg,
caggagacttaggcat, ccgtgccttccgggag, ttgctatgctggatta, ctaagtaattttcagc,
aacaagcttaaatcag, tcgtctggggcggggc, gagattaactctcttg, ttgtgaccccaggaag,
ttgaaaataatgcgag, gacctgtgccgcgtcc, gcacgcccttagaggg, tgtggatcctgtacct,
tggaaatacctaactg, tcgtcaccatgctgtt, agggctataaatgtct, tgtagatacaatgtct,
ccttattatgcaaagt, ggtgttcttagcattt, aagcgagttggtcact, ggagacgagggaggaa,
ttcatacattttcgct, tcagatcagacctttt, gtgaagtcaaacccag, cccccgaaaccctggt,
ctggttaaactaaggg, cttatacctgtgcccc, cccctttttttatagg, gggcctgtcgtctggt,
tgtgttcaggcgcaag, cggttaaaacctgtct, ccatttgcttgtcgtc, gcccccccccgatctg,
ccccggtaccttagcc, ggccctccaaggcatt, cgttgatcttgcgacc, gcactctctcgctctt,
tatagacattccggac, caaacgacaagtccag, cacgtaaggagtcatg, tggttcactgtgctcc,
gtagggctgggaaatg, agtattaccgtgtgag, acaatctctatcaatg, tactgattagcatgta,
ataagagcccccccct, tgtccattcttcaaac, acttagactggatgat, taagctatacttaaac,
atacccggggccacct, tacattctgtcatact, ggggagacgtgcaggt, caccagcggtggggct,
aattatgaagcttacc, ctttactctgtggggg, cgtcacacaaagaaaa, tcctatagaatagatg,
gcctgaatctgcgtta, gtgagggccaacctag, gcaggggggggggggg, gcaattgtttagactg,
ccttacagtgcaacaa, gacagttagacctgtc, tcgtcttctcattcgt, cttccttacagtggca,
aagctagggggggagc, tagttcgacaccagaa, acctttgatgatttag, catccccccattcata,
ttacacacccccccca, aggacagttcccctgt, gggggggattgagctg, acgtctggaactgggt,
ccctgatggccagtta, ctcccattccgcagac, ttacttatacacatca, taggggggggagggga,
tgaggggggggacaga, taatgatgggctgttt, taatggaactttgtgt, tgcgacctaaggcaaa,
cctacccctatcttga, agcaacgccgaagaca, ctccattaaccttaat, aggacatgaaccacct,
ggtctgaaattcccga, tgacttgcgaggcctg, ttacgtaacttatata, gtggtggagagccggg,
gcgccttccctgacac, ctagtctttttttagc, catcatccgatatgct, gacctggcttggtaga,
ctaggaatagccacag, agggcacttcttaagt, ggacattggggggaca, tggaaagcaacccgct,
acccgcccctacattg, cgagggggggggaagc, gacggttttttttatc, ctccaatgacttgcga,
cggagctaggctccgc, atggctctcagccaaa, tgcatgcggttgataa, atactccctctgggac,
accaggctttattaac, cctgcttttaaactcc, tcacgtctgcacaaga, ttcctcatgtgtgcca,
gtagagtttcactcac, tacctgaggagggcca, cacccagcgaaaaatc, aggcattagccgtcac,
atcattgctggcgcat, ggaaaatgttgcgcat, ctgcagttgggatgcc, ggggtcagttataaca,
gccattagccacactg, acacgcacagtgaaac, tcccccacatggctcg, ctgactattacttaat,
gcgggttgggaatgga, cacaccctctgtaggc, ggggggggtatcatgg, gcttacctttgaggat,
ccccccccaagggagg, agaccaagggccttag, aatggggctcgtctga, gcggccttgcgaccgc,
tgtcacccccccgaca, tttacccaactcggcc, gggtgattgttaatat, aacattttactaacgt,
ttagggggtaagattt, gatgtacaaggtttat, caagacacccaacgag, ggggggggggaacgct,
tttgtgattagtagtt, accgtttggacagaaa, tgagtctagtgggtgt, ccccggtagcgtgaag,
ttttattggagggggt, gctgaactacatactg, tatgttgggtgcaagg, ccgaaaaaaaaccaca,
aatgaagcttggtctg, ttagaggccaatgtca, cctagaccgggtggtg, tcgctttttttggtat,
gaaactttttgccaag, cctggggggggggcca, gcaaagtgtcaggtaa, gtactccttacttgga,
gaaagttaatgatatt, ttcaggacatatgcgt, gaccctaaaaaaagcc, aaacgggggagattgc,
catgcacttaccaagc, tatccaataatattag, gaggcgctcctgacat, gccggcacatcagagg,
ctgactaatgtgtcta, agcccacaaataacgc, gattgtgacgttaagt, atttcgagtctccttt,
atgcgaaacttgttcc, agtcattgcggctgga, cagcgaagagaatata, tctcttattaatagga,
tcggtcacacatgata, agtttcacaagtgtag, ataagatttaacctga, cttgctcccacgtcac,
actttaattgtgttga, acccaagataacctag, ataaggggagagcttc, cctttattcgattctt,
tgaggggggggcaaaa, cgcaaagcaagcaaga, tttacggtaatgcaag, tcattggcaagctaat,
tcttactctgcccaca, atcttacgggctgaag, tcttagtccatgtccc, tcttagtacccaagtc,
gcaaagcctggattac, tcgtccccctatctgg, attgcgtacatatttg, ctcacagggtaacttg,
cactccactaatacac, tactcagtgtacttat, acaagataagagcccc, ttctaattgtagagca,
tacgctaagttttttg, ttctgtcccgtgggtt, ttgcgaggtggcaggg, gtccagttgagaatca,
cataaagggcaaaact, tcatcccgcaccagtc, ctattctcgcaccatt, ttagagctacgctgca,
ttaaattccgcttgtg, acctcattactaggaa, tgacctagctgttagt, cccgctgaaggattgg,
aattatatcccgtgca, cagttttttttagacg, ctgtgaaacccttgtc, taagttaatgttagga,
agcatattggtcgtat, ccctgagaggccgccg, gcaatgacctaaacgc, gcagacatatggccaa,
ggtttcggctgaagtc, tcccatggtctttagg, cgttcaccatgaggct, gagcactactcctggt,
gggaagccacttatag, ggcgcttgtctaagcc, gggagtccttatccat, ggtgcaggatcagatg,
cctgtgttgtgctagt, aggtggtctcttcgtc, tgatgtttggataatg, aactgtccctagcata,
tccagtggttcgaaaa, tgcagtcttctgtcat, ctaaggcagcttaggc, cctggggcccctttaa,
tctaggacatgcacca, acagcgggcggcggac, cacagcaatacacggc, tgtcgctctgtcacta,
caggcattagccgtca, ccttacttggaccctt, agcactttaatctgat, atcttcaatctattcg,
tagacacgaagcattt, gggcaatggggggggg, ctcttccagtcatact, tccgatatgctctcca,
tgtcctaacaagttcg, ccactagagctggcct, tcttgtcaggtctccc, cgtaaaaaaaagccat,
ctgggatggtcctttt, ccactaaaaaaaaacg, tcccccccccgcttac, aggaccatgagttcca,
agagtgattggacata, gcttaggctgtgttct, tttttgtacaacggct, actgccatcccgagca,
ctaacttaggcagttt, ctcttgatcctccctc, gggcgggcacattgta, ctacacttaaatccca,
ttaagggaaacggggg, cttgtcctcaactagg, gctgtgaaaagcttgg, cttcgctgttatttcc,
cacttaggaccttggc, cggcaggcggaggtgc, cccccccgaaaaaatt, aagccatcggcaatgc,
atgacatttgtcagcg, atcacccttttagaaa, gcaaccatggaggtct, gggttgtcactgttct,
atagtcctcagtatag, taccatcgaggtctag, cgttacttatttttgg, ctcccagatgagggat,
gggataggggttcaag, cttaaagcattgccct, gtgactgccaagaatc, ctttctctggactcaa,
tggcccgaggcagatc, gcatgggtgctcaagg, tggctctttaccctac, tattatatgtgatcct,
aatacgaaacttattt, ggattgatggggggga, cgtttacaatgcggat, ctgagtacttatctgc,
tattgcgattgaagca, cagtcaattagtttag, atctaaaaaaacagtt, cgtaattcatattcac,
gccattacttaatgcc, ggaggggtgtgcatac, ccagcaacgtggaagg, gtgggattcttaatac,
tgatacgctctaagaa, accatgggggggggaa, cccgaaaaagtaatta, tacgtttgctttaggg,
atttaacgcattttat, ctaattgtttttgggc, ctcattaatgtcgcat, atattgctcaaccccc,
gttctagccttgaccc, ggttgacaggtgcccc, ccgtctccactgagac, agcaagatagccatcc,
atgggccctacaaagt, tcattagtccggttca, agacgggcttttagtg, atgcatgttagcgaat,
tagcttccccccccag, tgttttttttcgctaa, gtgaatgattttgaga, tctccctctttactat,
ctgattgggctaggcc, caaaccttaaattacc, cacagtttcagttcgc, ttcctacttgacgaat,
ccactcggcagtgctc, ggccaggcggtccgcg, gccatctttgatctag, ccctcatgtttgcaga,
ggcttctgcctaatat, tcaataagccccaaaa, cttatgcccccacctc, taactgtgatccgttg,
gtgtgtggtctccagt, atgtcgctggatctcc, ggaggcggggcctctt, ggttatgacccctgaa,
ccgggtcttaccccca, taattgactaactgcc, ctttgcatggtagccc, tatccaggttgttctg,
acggctggccgggcga, gttagaacacgcggtg, cttaacgtcacgcttt, gaaactacgttacact,
ttcggggttttgatat, tttctttggcaacgca, aaagcctttattacac, caccaaaggtcaactc,
tagcacaaggaccaac, aaaaaacgaagcccat, tgccggtcttgtcggt, ccggtgttcttcaggg,
gatgctactggtgcct, ttgagccggctgagat, ctccggaatggtttag, ggccttcaaatatgtc,
gactgaggagcattgc, ctaggccgccgcactc, ggtgtccttgtgtgac, ctgcgtagagatatga,
aaaaacggggggttct, gagccccgcacacacc, gagaacccttgctact, ctgataactaagtagt,
aggaggctgcgaagtc, atgaaacggattcaga, tgtttcgagggctcag, tgggggggggacagct,
ttctcctgtgtaatag, gcagacggaggtgcct, ggatagttaagtaagc, tgataatagactacca,
ttctcatatatgacac, ccagtcagctgattta, agtgcccgccacaagt, tgacgcctgcgtgtac,
ttacatgaagattgtc, agacgttaagggaact, tatcctggagcataag, gagccgcctgccccta,
cttgactcagcttacc, gccgtgctttccgggg, taaaactgtagtgtcc, agcgtgtaacagtacc,
tttagggggggatgag, cacttgataattacga, ttagtagttcttactt, ttacaggtccctatat,
tagtatgggggggaac, tcccatatagcatatc, ataaaatgtcggaaat, agctcgggggcgaaaa,
ggttatttgtacaagg, gagcttgcggacaacc, gctattaggttgcaaa, gaggtacctcgggggg,
cctatatggagaggcc, aatcggtgatttgtct, tgttcgggttatgagt, ctaattatagagataa,
aattgtgcatgtgatt, ctggggtgttacatat, ggtttgtgttcgccct, ctccagttgtacacat,
ctcctgtaagttatgc, gattactccattaacc, tgcgagaaggcagaac, taagtgataggtaaag,
gatctagtggccctta, cattaggtcagcttcc, ccaggacgctcccatc, gtgacttcttaattcg,
caagaccgactcatct, agttacggaaattaag, cagcagttgtacccct, aggtacaagcggtttc,
tatcattacgaatatt, accttcgggcagcaat, gagggacaactagatg, cacagctttttttgcc,
cacatcccattgtgtt, ataacatgtgacttat, acattcaaaggacacg, cgcagaagcctgggag,
attagtcctgggcact, ttcggctctcacacgg, atcgcgtggtgacagg, cccgcacacacctgag,
taggatgctgttcacg, cgggtttgttcctcgc, ggtggactgtggacgg, caacattggagagtcc,
ttaagtagcaagtgct, ttcatgtgtggacttc, ttacattgcgtctctg, gtgctctcttaaacat,
agtattacccatatgc, ccacttgctoccacgt, ggtcaaccttgccaat, ttaagaagttctcggt,
catgcttcggtgtggt, aagcactttagtaggt, tctcgctgtcttcacc, tagtcactgttcgtag,
ataagtgctgtacttg, atcccctctactcggg, gcagatgagggatatc, cacgccattagatatt,
gtagaccccgtctgta, atataagctattaagg, gtctacttatgaaacg, cgaggtcctgcttcct,
cagccatcataagcct, ctaccatacaggctga, gcttggacttttttta, acccttggtggcacat,
cttgatggattggtgg, ttgtatcttgacactc, tcttccgtcctttgtg, ctctctcgctgcaaca,
tgttacatagagatta, ctctaacaagcactcc, ggcaatggggggggga, ctgacgcctgcgtgta,
ccgagatggctccacg, tactcacctccttaag, agctgcatgttagccg, tccgcatggtgcgagg,
ttaccagcacttaaag, ccaatagcaacagaac, tttagagtattcaatg, gtcaaatgcttagatt,
acggaagagtcacata, cgactctcacttctat, cttagagggagcgtct, ccctgtggtatctggt,
gagggtcaggggctag, tgtcggtggaaagtca, ctgcgataaatgttaa, gttgtaggaggcagtt,
ggctaaccagtgcacc, ccactcactaactaag, gcgcctacttacacag, acgctggccctcaagc,
gcagcagaaagtgctt, ctattaaataggggta, cgccggtgttcccttt, ctggattcgagactct,
ggagctgttagaagac, gggccacaggcccgat, agacggcttttttttc, gtaagaactcacaaga,
taatatgctgcgctta, ataacaaacgttatcc, acacctgggtaagttg, acactgttcctcttga,
ggatcagaggcacgtc, ccagcaaatgccatga, tcttcactatttaaca, gcattttgcccctgta,
ctaagcggtgaggatg, atggggtttaacgtgt, gaacggatcctggggg, ggaccccccccaccgt,
ggcatggatcccacgt, acctcccccccccagt, gagaattattcaccat, gggacattctgggtat,
agggttccgtcttaaa, acgaaagaaacactca, gtggtggatcctaaga, cacattacctccgcat,
tgttcacccctatggc, ttgaagtggtggcgta, aataaccttcacgatc, tttagctgcagccgga,
agggtaacatagctgt, cttataacaagactct, gtagagggggggggaa, gaaaactcgtaagcac,
aaaatggttaccgcaa, ctcactgtgttttcgg, ctatcctgggcaacga, gtcctcatggcctaac,
ctaggccgccgacaag, caagtacatctatcca, cactgtgtgtttgacc, acctccgcattgttat,
cttatgagaaccattt, cactcagggaggctta, tcaaagtttaactaga, aaccgttgatcttgcg,
gccatttttttatata, gttaggtggagggcaa, acataggtatacactc, tacctggtattcacta,
tacgtaaaaaaaaggt, ccccctttccccgaag, ggctaaaccccgtgtc, taatcgtccatggaac,
cacgctcccagatgag, ggacatatcattggca, gggaggtgccggtgtg, cggaggaaaagcgtag,
ataaacctgggaacac, ggcattgaccaatgag, aggcactgtcactcaa, tgtacctgagctgttc,
gcgcgcggggctgtcc, aaaggcagttatcgac, tgtccggagaatacca, gcttaaagccccctta,
aggtggattccatatg, ccatatacttgaaggt, atcgttaaaatgtaaa, gctcttagcctccaac,
tccttaattaacatcg, gttcaactaactcagt, aaagaagatgactgcg, ctgtagcatattgata,
cggagctccgcgcggg, ctctttttttcgatgc, gactacaagcgcactc, agtcattctttggaca,
ttatcctttattagac, gctttttgtagctaac, gtatgagatttggtgc, cgggcgtggtgacgct,
actcaccgcatgacga, agtccatgtgctccta, acgttaccagtggaca, tagtgtagtgagtctg,
aaatccttaaggtatc, atcaaaaaaaagggcc, aaccttaaaaaaaacg, ttatccgcatatttct,
caactgatgcatcatg, atgctggaaagcagcg, ctgcgcaggactatcc, cgccgtagtcggcgtg,
ataaacgcaaacaaga, agagatttatgcattg, tgctggctttagacca, cgcatcaaaaaaaagg,
aagcgtagcgaggcgc, ccagtattgtacagat, gtccagaattggaggt, agacgagtaggttatg,
tcttgcccgccgcgga, gcttctatgccccccc, agcgcggggcccttgg, tttcagagtcacgaaa,
gcacatcggacttgat, ccttagttttgagcca, tggaatagacctttaa, cattccgcgctccgcg,
agtgtgacaattttga, cggcgtaggataaaat, ttccaatgactgttgc, ccaacattgtgtggac,
ttaaacttaagttctc, ccgtctagggacaaaa, ggtctaagacaagcta, ttgcaccatcctctcc,
tgtctgaaaatggttg, actctgccccccccag, taaatcttaagccgtg, ttcacaatgctgtagt,
ctcatgattctagttt, aagtggtagctataca, gaagggacatagggat, attgtgccttaaaggc,
tgtgccccccgaaaaa, gaactgatcacctacc, attacggataatttgc, gccggaacagttatct,
gatgccctgcccgtaa, tacccccccataccta, ctggacgccccccccc, ttacgtggtggataat,
acgtttacaatgcgga, actttaccatgataat, tgctcgtaaaagtcat, cccgagtgaccgagga,
aaggttagacttgggg, ggtgtctctaaataag, ataaacacaacttccc, ggagcagcacttagcc,
tctcaacactatcctc, cccgctttccatcgtt, gataaaaaatttatcg, agttcatagtaagggc,
acgggggagcgcgtga, taggccagtttaccct, acttaatgccttgatc, ccggaccccgaggggt,
cctacagtaaggctgg, actgccagctgcgcct, acggccatctctacaa, ttttgctatttcgcag,
tctcggtttcggctga, tcaaggtggagcactt, tgttttattccctacg, ttgttagggatctggc,
atctcttgcttattag, ttctccagggaggcta, gatgtattagctaaag, acggggggttctcttt,
tccggtagaggtgaga, ctatcacctgatgact, actgctcccgaccctt, tcaccctaaaaaattg,
toggcaaacttaggca, ctctgtaaccgcttac, ttagaagggtatgggg, gagagaagtgcagcgt,
gtaaaggataagaggc, ctctgaatatgtgtta, aagttcatcccgcacc, ctcaattgtttgggca,
ccttttaggtagccta, tttaaaaacggggcta, ttatcagccataagac, ctgatggtgtaaatcg,
aacgtccagccttaaa, gggagttctggttatt, agtacccccccttttg, ccatgatgggggggga,
gcatgaataaattctg, ctagggttgagaccga, ccacccttggtggcac, tgtcaaaaaaaagcga,
agtgggggggggacta, tacctggatgacagcc, cggagcagcgtctttg, attaattattttcccg,
tgtgcccggaccccga, accatcccccccagtt, ggcaggatcgtccctt, tagaccttcatggttt,
tttcattagtccggtt, gttccaaatccctggg, tgaggtgatgagttac, tatgctggcaccacat,
gcaagtacgatgtctt, cctggagccgggagcg, acactgtcaaacgtat, actgtcactcaaagag,
acgggtggatatcctg, caaggtgtaacagtag, cagtctgaccccccca, tctgaaacgagctgcc,
tctatgtccacgcgta, ctgacacctcttagga, ggtgggccgtgaaggt, cataattgtacagggc,
tgcggcttagcgccgc, taaccaggcatttcca, gctctcgtcttccctg, gctaataaatctggat,
ggtttagttacccaca, tctaaggggggggatg, agtggataacgaaatt, cctttactgggttatc,
atggtcttatgcactc, ttccaataggcccagg, gcaactatgtcatgtt, gctcccgtgggtgcca,
ctcgtttgtactaaaa, aaaaaaggggcgggat, gggggggtcagaggtt, gaggctatgtaacagg,
attcaccgttttttta, agaaatagtctggtat, actggctgtagggggg, gctgaataacttcagc,
gcccctatctataaaa, gcaatactagatcaac, accccccccagcaatc, aatccteggacccctg,
gatctagactagtttg, cctcctcttaacagca, cctatgtgctctgatg, ggttaatctggttgtt,
gctgcataaagtagtg, ttggcttaaagccaag, aatcgtctcctcctcc, tgaagtacactggctt,
tattattataacgatc, ttagaagtagtcttgc, aacttacaccttgtta, tacttcacatgtcatc,
cccacacaacattgtg, aggtaatgtattctga, tattaatcgtccatgg, gctggccttatggatt,
ctccaggtccgtcact, acaaatgttagacaga, aatagcatacaaatag, atctccccattcattg,
tattgtccctcctgaa, attctctccttccgac, aatcaaaagcataagt, aatgcacgccgtagtc,
taaaatcttagtacga, tcaacatgtgacccta, agggattatcaattat, tcctgagccttagcag,
tgctccagcttagctt, tcccactattccccgt, tggtgcactcagttgt, acccatctccgtaact,
ctagttcatagtaagg, gagctgtcacagagta, ggggctcgggcttacc, ggggcgaccaaggcct,
ttttctgacctacgag, aacgattgtctcctgt, tagactgagctgtatg, agatggaacacttaga,
acgcagcatcatctat, gcattgtcatcacagt, cgtctgaaaagacaag, gattacttcattgcac,
tgttagagaacatgtg, cagggggggcaaaaaa, agcatagattataatc, cacaaggatgaggttt,
cctgatgacatgccta, cacctgaaggcactta, tgtttttaacgtgccg, ggcttcctaagctgtg,
gatcccttccaagctt, ggtagcgtgaagcctg, cttcttcaccaaccta, ttgataacatgctatt,
gcagctcgtgcttaat, ccacatacgtggtgcg, tagctgcggcctgcgc, gacctcccgaagcagg,
attgagagtgcaaact, tagccggggatctctg, tgtgtcaaacacaacg, catctattggatgctc,
ttagtggggagtgaat, gatttacccaactcgg, aaacgttctttcactt, tgcagtccctctgcac,
ggctcataaaggttta, tggacgctaccggcct, gccaagctaggcttaa, actgcggaggcctagg,
ctggcataaagccagg, ttgaaggctgatgggt, taaggggggggatgtg, tgaaacgagctgccaa,
ccaaactagaagtatt, atttcatctgaacaca, caactactgggtatta, aaatgaccagccaata,
tggacacgaaagaaaa, tcccctccgtgttgtg, ctcacctccttgtttt, caagttttttccacag,
ggttaaaacatcccag, aagcgagcaagaggta, atgaagcttggtctgg, gttagtcatcctttta,
tcctatctcattagta, agaattagcagatggg, tccctttgtaaactag, ttattctgggaagttg,
tcggttaatacggcaa, gacgatacgcgagccc, cttcccccggggggag, gttaattgagaggttt,
ttgggggggcaaagaa, catgtctgccaatggt, gaggggggggcaaaag, ttcttaagttcatagt,
tgctgaccctcttagg, atctggtgcaaccttg, taccagtgctaagtga, atctctttgtattagc,
agctttttttaggtct, ccggtagcccacagca, aggaacggaatgaaag, ttggttacacccgctg,
tcaaaacactgagacc, gaataagccaatacag, gagagcgagcaactgt, ccacgtgcttctaatc,
cgctcatggtgcgctg, taggtgggaggcttag, ggtgcggaaaaaaaat, gtcgcaggagtagcca,
cgttactctgtctcaa, acagcctgttggttac, attgcaagttgcaaat, tgctgactccttagat,
taacgcccattttatt, tagcgtagctcgaagg, ccagactgcttttaga, cccagtgtgagcgact,
ctagttaactccctaa, cttccgtacaatgtac, agtgcactggtcttca, cccagtattcctgggc,
gcttatctaggcctct, gcaatggtattttttg, cgagattcctgtttga, agaacgttgtcatttc,
aagaggcgctaagccc, gcagacccagggttcc, catttgatccatgaga, gcaagatgctccccca,
tagaacagggttactg, catccgaaggctcacc, tctgacgccactccaa, tttatgatttggctac,
ctcggataagacgctg, aacaccataagtcatg, gtgctcccccccaaca, aaggtttgttcctact,
gaacgaaatccaaatg, tacaccgcagtaacga, aagactaatgaacgtg, aaggttccccccctta,
cgcacggtgcgtacac, tagacctttatcactc, tagctccagaccaagg, tctgtgcggcctttcc,
tgaactatttcccgtt, caccccccgaaaaaat, tcccccccccagacaa, gcggcggagctaggct,
gagactggggcgctgg, tgaggctgtcaaagcc, agtaaaacaccatcag, ctgattttcctgccga,
taaattttaccgtttt, cccattccgcagacac, ttataacagagagtca, aaagacaagagtcatc,
actgcagttatccggt, ccatacttaagcttat, gtgattctgggatgac, gcaaatctgcccttta,
aaaaacggggggttca, catcatccagccaaga, agcctctcctccttag, ccgcacctccctgtga,
cttatgaggaattatc, acgtggtggaatggca, gcggccttgccgcgcg, tgcctactctgattaa,
gtaccctttatttaag, getaggttattcagct, ctattagattatggtt, aggaaggattgagccg,
gtgtaaaattgtgacc, tccgcatgcagctgac, tcctgccaccgtggtg, gtgtgaaacccattcc,
tcctctataattgtgg, agaagacccaccagca, aaagctaccccccacc, acccactttttagctc,
tatgaccaatgggtct, gcggtgttttctgccg, gcatttgttatactaa, tacctgtgcccctcaa,
gtgtctttgctgagag, taagattatattgagg, caaattgtaaccctaa, gttatttatggtttga,
gttaaaaaaaggctct, ttgggtggcttaggcg, gctacggatgacctcg, cagtgtttgtcgctca,
cggcacggctggcccg, ccctcggttggtttgt, gttacaggaacctttg, ccctttctctccggag,
gcaaaaagcgttcaga, caggcacttgccgcag, gggcttaaactcccaa, gcactttgctgggccg,
ccggaattgaaaaata, tcacttttatggggcg, atgctccgccttacta, agtgccacttccctcc,
gggaatgatgtcccga, gcgctctgatctaaac, caagttattagggggt, cctgcctaaaaaaagg,
catatggttaaacccc, gcgtttgtttaaccat, gtccccccccccagaa, tctacagacccatctt,
aaatttacgtaagtat, aatgcggtaaaacaga, agtccatctggcatgt, ttagtggtttactgaa,
tccatcactgacagga, ccttttgggtccatat, gagccggcaggtgtac, tactgtatacttcctg,
ataatatacaggtggg, agcaacagagtcggtt, gccataggaggatgtt, tgatggctacgttttg,
gcttttttttacatcc, ccacgagacagcggga, agaatcccacaagcgc, acccgcgggcgctttc,
tgcaccaaacgtctgc, atgtcaactactggtg, gtggccaacattagtc, agctaccctgtctact,
tttttttcgaaattag, ccctaagtcctgttag, cacgctggaaccacag, cggtagtccagtagag,
actgttgataaaaaag, acgctactgtaaatag, ggccgtagcttagtgc, ttactgattagcatgt,
gtcagtttagcctcat, gcaggttaggacagaa, gaaacccacccctaga, atcaaccttttttcaa,
ctggtatgtaactcct, ccacggcttaaagcta, gacgcagagagaccct, taatatgaaacggtaa,
actggtgcttgtgatc, gacgatgcagcggatc, attatcccgggggggc, gacggggggggttaca,
gtttacgctgggccac, gcaacattgatagcat, acttatgaaacgcaaa, cttgtacgatctcgtc,
cacccgcccctacatt, tagatcaaagacacac, cgggttttttttaagc, ccctctcaggtatgtg,
acattgtagccctaaa, ccaaatttcactctcg, ccgttcccggctaact, cggccagaggtgctag,
gactgagtagacttca, gtacaccacgggatat, taacattaggcttttc, acacggctgtggtgct,
tacccggggccacctg, aggacactagatccaa, gaatatttttacaccg, gttggggcccttctag,
ctcagaatttggttgc, ttaaatgaaatcgtga, aacgcggggtggactt, ccacaatcaggggcac,
ccactcttacacaacc, tgggctgtggttaggg, ttatatccatgttatc, cacggaaaactccgcc,
catcactgttatccaa, ttccaaggaccattca, atccaaggagacgtat, ctcatcactgattgct,
caaatgtaactcgtgt, gcctagttcccagaca, tatgtctgttagggtg, agtctttaactcctta,
gatttcaccaaatgta, ctgtagcttagagcgg, gtctgaaacgagctgc, caagattttttggcaa,
ccgatctgccctcgcc, ttagaggatcaattga, tgtaggacctcccctg, ctgatgacatgcttcg,
tgagcagcattggaaa, ggtagacagctgcctc, acattctcatgaatag, cagggagacggccatc,
gacggcattttgacct, gaccagttaaaaaaac, tgtgttaaaaaaagtc, agtttctctattcagc,
attcatcgacacatgg, ccgcttttttttcaag, gacctcccctgtcatt, cacctaccctccttgg,
ctgagacattcccaac, cttagattgccccact, cgggaatgcacgccgt, aaagcgactgtttagt,
gtacatgccagtccaa, gcaagctccctatggc, cccacctctgacttta, gggatccccgtgtatc,
gccggataaggaagtt, gcttaacatgcatgtt, agtttcgttcctgttc, tccaatatacaccggg,
tgtgggctacagttat, gccaaaaaaggggggg, gatatccaacacccac, ttctgccttgattcgt,
tagcagagagtaaacc, catccaacggatgtag, accaaaagtaggattt, gcttatgtcctgactg,
ttacaggcttggtcta, ctaattctgcagatag, tacaggaggcttaggc, ggcgttatcctgggag,
gactgaaatgcatggc, ttagtcctgatgtgca, agactggccctttggc, cgctcaggaaatcgaa,
acagtcagctgtgaga, aactgtgtagaggtag, ggactccccacttctg, tcatggaaaattaatc,
gtcggccccgccccgt, tctgtaaccgcttacc, ccaagcaactccttct, ttatctacctcggtat,
ccgcatgacgaggtcc, gcattttaccccgccc, agtattgaagtacttg, acagatgtatgtccag,
tgatgtaaatcaaacc, tgaagagttcgagaca, atggttagacccccgt, cactcgtgtactatct,
tgcaatccagattaca, atcagcgtattttaat, ataccagccctcctta, cacacttatattatac,
tcgatttttttttgac, aaacctgaaagttcgg, cggtgcgcgggacttc, agtataatactctcta,
ccttgaaagtcttgtt, cattggctgtggtcaa, tatgaagactagcaac, aagggggggggtgtta,
gggcattagtaaaaga, ccagggaaaaaaaagc, ctgctgagttaggatg, tgtgtaaatctggcat,
agagaatttacaggcc, agacgcagaggaccat, cctgggattgaagtct, acgattgtctcctgtg,
aatggtgattaacccc, tgcaaaaagtcaaacc, cttaaacagccccacc, tattatggcgttctca,
gatagcattgaatatt, ccttgaaggtgtcacc, ctcctttagaggccaa, gcggcggaccttcctc,
tattttttgccccccg, acactgcaaatgttgg, tagtcctacacaaata, attgattatgttgtag,
agcgttctcatttttc, tcccagcttttttacc, ggcagattagccttca, aatcactggccgtttt,
atatggcctgtaacct, tgtcaatcctgggcct, gactgcagtgtcgcaa, gacggtagttttttgg,
gggggggggaagagtc, gggggggggtaagctg, agaagagattactatc, gggaatgaacatgggt,
ggcccagctattttgc, gaatattgtaatgctc, aaaaaacggacccagg, ctttggtggatacttg,
aagtctctctttatat, ggcactctccaccata, ctcgttatccgctcgc, attagtctggatccag,
ctaaaaaatgctaggg, ttcgtcgggacgtccc, atgacaaccttaaact, cagtttttttgtgcac,
atggctccagggtatg, gtgggggggcgagtgc, gttcagtgtctccaaa, acagttgccgaataca,
ccgcaacaaaaaaaac, ccgctcaagctgttaa, gttctctaaactcctt, agagttgtctgaccct,
ccggaaatcaaaatta, ttggcccggtggccct, tgacccccccctcact, toccaccagtaaacgc,
acggggggtggtggga, tcgcggccttggaacg, cgagaccacacttcca, cacaaaaagaacggac,
tgatgctcttaacagg, gttaagtaagcatctc, tacacactcatagaag, ataagtccccattaca,
aagtagacatattagc, tagagctacgctgcac, ccgccagegcattacc, tactgcctccatctcg,
gaggaaccccccacgc, cgggcgccccccgcta, aatataaacaaaacgg, gctattctcgcaccat,
gcaatagatttctatg, actgtgccaaaaaagg, acacgccccttgatat, ggagcacctgtgcccc,
cgtcctcacctcacac, cttgaggacctgcttt, caccttttccacttgc, agaccttcttgctaac,
aacatctttcttcgac, atgtttgaactctcac, atccctatttactaat, ccgggttttttttaag,
ctcgtcttgcgggtga, tcccggggggagacga, ccatacttctgccata, ccccgaactatttttc,
gaaaactttccagcgc, actaaaactagggttt, ggttgtttcgttatct, tggtctaactttctta,
tccaaccatcctgtgc, attaagtagcaagtgc, tatcccctcttagggg, atcttttaggaagaac,
acacaaagcgagcaag, agagcactactcctgg, agcttttttaggcctg, cagattctataactta,
aacaccattatactac, tgtacccccccagtta, ttccagtagatcgaat, cttaataattctaaac,
tccccccacccgaaaa, tatgcatgttagcgaa, aacctttctgagccgg, tgatttcgttatttgt,
ataacgggggatggat, gagctgaaatgtcaga, actcacgtgatggcag, tagtgggcaaagggcg,
taagctcggtcacaca, gagacaagataagagc, ataaccctgaactagt, atacttgtgccaggtt,
atcgttgatccttgat, aagaacttagatacta, agtggtgcttttttta, gcaagactctatgtaa,
gtgggggggcacaagc, acttgggagcgcagga, ggttgtaatttggtaa, ctcagttgagcctgat,
ggggaggagcttagag, gcacaccaacttgaat, tccaggtaggtacagg, caggaaatttgtaggt,
acaatctgacgttttt, cttcctacttatcccc, gcttaacagcagccaa, gcaatgtttagcagat,
ttagggcatgattgga, accccctgagaaccct, atggcagcttaattgg, ttcaagagtgaggtag,
atcactggtgtgtaag, cgattggtccttttgt, tacgtcaatttttttg, agtgttcctactcaat,
ccacagtcggcctttt, tctcccagagaaccct, agagacgatgcagcgg, cacgaagaagacgagg,
ggtcgcaggagtagcc, acaagtaagttactaa, ctgtatgttgcataaa, cccgaaaaaaaaagat,
cgagtccaacgttttc, ctagtagttgccaggc, aatggtctggcagctt, ggttgggtgttagaaa,
tttatgtatgcccgca, gaccttactaaaaatc, taagactctctggagg, gcccaggggggggcca,
tagactgtgacgtgtg, aaaggacagctacttg, cgcccccccccatcac, cgtgcggagccgaagc,
ggtccacgctccctag, ctcttaaggattggcc, gaaatttccccgaact, cttagctcaagcgctc,
caccagtaagcttaat, aaactcagcgtttcat, ttcacgctggaaagat, gagatcttagaacggg,
actgacggtcacactg, tgattccatagagtga, ctcgccccggtggcag, ctaagagggacatagt,
gtaaaaaaagtggctt, gatcagcgtcaatgcc, aagcaagctccctatg, aaaaaacgagagtata,
gttggcaaaaaaacgg, tacaggcgcgtttcac, gcatacaagggtctga, accaaacttatatgct,
aagtggattgcagcaa, taaaaaaacctccaag, tcatccaacggatgta, gaagtctgattaaaac,
taggacgtgaggagcg, attagtcagccctctt, agagcttgcctgtaat, agtacggaactcattt,
gctttctaaccccccg, ttttagttttatacga, cgggaccgggcgctag, gtcccattctatgctg,
ccccccccaccgaaaa, gatgttttacacacca, ccaagatgggaggttg, gaaacgagaaataaag,
ctcgcggcgcttgcgg, agaactggtgcttatc, cctgcatatcttccta, caacgaggctactgag,
gcatcatatgatgacg, tgggcttaagctatgc, caacgataattaaata, ttgtctcgctgaaata,
gtccctgcttattttg, gacatgaactccctca, caccgcatttattaaa, attgagccggctgaga,
cagtgctgcactaagc, caacccccccagccta, accgcccctgcctgct, acctagtcactttgaa,
ggagaagttgtagtga, tagcttttaaggggtg, ctaggaggcttaggcg, ttagccagtatactct,
tccctctctgggttga, agtgcacctgatcatc, agcctcctgtagatga, cggtttttttcccctg,
agccaagtatgggata, gcagtagggcatggac, accgccacccggagcc, ggcctaggttgtgttg,
aacggggggggaaaag, ttgcctcactgcgacg, cggataatagtttaaa, tttagggggggcaggc,
cgcccgaacacttggt, cccgcgggcgctttcc, tttactgttacgagaa, ccccatttatttaagg,
aagtgaatcttagcac, gccttggttgctcagc, ttttgcaccgcttgtg, ttgttatgtccttcgc,
ctccagaccgtgcggc, aggcattagaatcgtt, attgcctgcgcaggac, cacgggctcttttcct,
ctcttatcacagttcc, tgcatacctggagctg, ttattagcaagagtgg, gccctgctataacaag,
cccaagctattaatat, ttatactcatgtatcc, ggggggctgactatgt, ccactcagtcagacca,
ggagaacccttagtgg, agagggtttgaccctg, aagtagcccagtcagt, gtcattgaacagcacc,
gtgaattgcacccctg, tcactctcttcgttta, tctaagtagttatcat, tcattacttgtaggaa,
cgtacacacacttact, acagaccatgacaacc, agcttaaccagctggg, ttcaatttcaccggct,
gcggtctgggtttgtg, agaggaaccgcccacc, aattccgtactgatgc, acctggcagtagaact,
actgcttggaatacta, tcacgtccttgcactc, ggcgtgtgggaatgct, tggtggagagccgggg,
taccaacgacagccac, aatgtaaggaggctgg, tgattccttcaccaac, attcgggccattctgt,
ttttgcaggcttagtg, ggcagacaaaccttag, gtgacctatccaaaaa, cgaggtctagttcatc,
gacggcttcagtggtg, gcagcacaagtatgcc, agaccacttattgccc, agaagccgcaaagagt,
aaacccgactctactc, gtccgtctcgccttgg, ccatgcttcgcttcgg, acctgttgagcccagg,
caagatagctaaatta, ccaaccaaggacaatg, atgattcttccaccgg, tcttaactggctttgc,
tagaacgtagactaca, tggacccccccatata, cctcctggtctcttcg, cacctctctgccatat,
agtgataatcgtgtac, gtgcattgtcccagca, ttgctctcataccctt, gttcctgtccaaggtt,
agcatgctctgcaatg, tatgtactcacgacct, attcccattaggctac, ttcattgaccccccca,
aggtgtgggccacttt, tcttaatggtatctcg, gatagcaccatcatct, gtaatgtagtaggcag,
ccctaagaggggcgga, cccctgtttattcaaa, agcagtcctgaattta, ctctcgttctgcagag,
ctctaggcatttggta, gtccatagctgccgac, aactaacgcattgttc, ttggatcaattatctg,
gcagatttctcggaag, cacgcccttagagggg, tcctgtgagtgcttca, ctgatctcagtatcca,
aggtgaataccatgtt, ccacacacttaaactt, ttcatccgaagtccga, gattgtgcaatttgat,
gttacctttgcaacag, gggtaatctcaattaa, acgaaatttttaatga, ctttctactatgttag,
gagactcgtcttgcgg, cccttcttcgccacta, gattggggggggaata, atgataatagactacc,
ctcaggagacttaaac, gaacgtagactacaat, agggcggccttgccgc, aacagatctgcattcc,
atgagcccccgaatga, gtccacaaaagatctt, tcaggtcacccccccg, cgcccaaaacttttag,
gactcctctagttgta, cctcagcgcttctctg, ggcgatttttttgggg, ggccatagaccagcga,
gttcacagatacctat, atgcacctgctcgtaa, ctcgtgaaccctggag, attttgattgatcaga,
gtggcgttttttttcc, gcaaaaaccctttgtt, tcggcagtgctccagt, ggtcacacatgatagc,
tcgcagtcacctgtgt, atagcatctggcaatt, taggtattagtccttc, taccttgatttacatg,
ttgtttgcgtttccct, ccaggttagaactgag, aaaggtgcaagctatt, tcagaggcttactagg,
gtcggggatgggcctg, acttattaaagcatga, agaatccaggaccaaa, gcattgtctaatctca,
acagcttatagcagcg, ctgtatcctgagcttg, ctgatgcacgggctct, aggtgtacaatagtca,
tccacatgagtcagcc, ggattccaaaaaaacg, tgtacttctgatttcg, aatgtcgcatctattt,
ttagcttaggccaggc, tggctcactttatcct, ctgttctcctaacaac, tcaggcagtcaagccc,
taggaactcagacagg, aatctcagactaggtt, tgagtgtgagattaga, ctgcttactctaacag,
cacctaatttaaatga, ctaccccataagttat, gcacctgccgtgccgg, tacctagaattaatac,
tactttccctgttagg, tgattttcctgccgag, ggcggggttgtggaat, cttttatgttacccct,
gaagccgcatcccgct, ggtcttcctgggtgaa, gagagacatgtctaac, cctatgagagcattac,
caccaataagtcttac, tgcggaatggggtaag, cgttactagtctgtga, ggctgttatgtttctg,
agaccgtttttttttc, agctcacaattatgag, ccttgaggtgtttcta, agcttgttaaaatcag,
cagtttttttatcaac, tgacagacaaacatag, tgatgccgagcctaag, attgcctcgtccattt,
aacgaagcccattttc, agccaggaataggtaa, tctcaacacatattga, gattgctgagtaatcc,
tataggggtaatttag, agtaggagaacttcgt, tctgaagcacggcagc, attcgttagaactcca,
gatcctggggcatgtt, atttgagactgtatgt, acaagccatcggcaat, caagccatcggcaatg,
gcacttaacgaggcca, tacctatgtattgatg, tttacaatctgacgtt, ctcctatgggtctccg,
ttcgtttggtcacttc, ctaactaggccgccga, cggaatccccaaggtc, ggcgtctacacagcgc,
cattgtgtttccgtca, tttaacatggatattc, gaagaagcttaggccc, tttggagcaccattta,
ttccagaatcttatca, aggctgccgctagcgc, gacccgcgggcgcttt, ttattatagcctcgct,
cttcctcgctggcgat, atcttcttacaagact, ctatctcagtatgcag, cgccctcgcccctggg,
ggtgtcatagtgacta, aggtgggcggtccttc, gatggaggtctgacta, aagaggtgtttttgga,
gctgctacataagtgc, tgctgatccgctgtac, aactgatgcatcatgg, ccggatgtatacagta,
attaggtgggatgcca, gtggtgtcccattaac, aatggggaggccttag, acgacaggggcccacc,
cgggtgtctccctgta, cgaacatgttgccgta, ccacacgtgtgcatcc, tcctctcgacttctaa,
catcaaatgtctgaat, gccacgcaggtttccc, aaaattctagggtacc, ttcgatgttttttttg,
taatgcaaggggggtg, gcttttgctgcgcagc, ggggaaaactccatca, tcaactattgacaatg,
cagaaattaccatggg, tcgtcttgggagagag, caatgtagccacaaag, ctatctattcggttcc,
ataactcttacaacca, cctgatattgaagatg, ttatccggtagaggtg, gacaatcactcaaaag,
cacctgagtaagatgg, cagcgtcggccccgcc, ctgcgggtggacaatt, aatcctcacctctatc,
gcagagagttaatctg, gcacctcttgctctgt, tccaggatgcgcagtg, acctagcatccttaca,
agtcttgtcatgtata, tcatctgtcccccctc, acteggataagacgct, caacccactggcattt,
ggagactattgcagca, cagtaagggcagttac, tacacacatctgagga, ggcaaataggccccct,
agaaaacaacgatcta, cgcctttgcggggggc, tggcatagacgctgct, agagagtgtcttgcgt,
atgtgaccccctcttt, tgcaaccccccgaaaa, aggactctgcgatgca, tccttcgctggcattc,
atattagaaaaaccgt, cacgctaaaaagtttt, tttcgaaattagggat, ccacctaagctgtgac,
gcttggtagttatgaa, agttggggggggttca, aatgaagtctagcctc, ttataacgtttagtga,
caagctggactttaac, gcatacgaagttcaga, tgctctgtaaccgctt, tgcagcccgtccggtg,
gcaacggaatgtggga, aaagcaagctccctat, gtacagatgcattgtt, attagcctgatgcgat,
aaagtctaattactag, cagccggatgtgggtg, gggcataggcccagac, ccgttacttttatctc,
aaggctgcataaagta, gaggaaagaggcgtat, tataccctgcctacct, aaccaacaattaggag,
ccctttcaggaaagat, ctccatagtgggagcc, ataatccttactggcc, cacggctcagtgccct,
aggagacggggggggt, ctgtactcgcagttag, gctccacactaaggat, atggcggtgaagttca,
aacgggggagattgca, ctctgggacacttatt, acggttcaaatgcagg, ggttgtatagtactgc,
ttctgtgtcgcccacg, gaagaataagcttggc, tttccaaaaaaaggcg, cgatgtaggagtcaga,
cggacctgttttcatc, cgtgaggagaaccgcc, gattttgtagggctat, tctcatttattcaggc,
aatccttagtttacca, gccttgttatgtcata, taacatagagtttggc, tcagggtctgctttac,
actgaatactactatt, aatgtttgacgtgtgg, gcaacaggataatagt, gtccatgaagttgtaa,
catgaaccaggatgcc, gactcgtcttgcgggt, gtgggcggcttcctcg, actttttacaataagg,
ggtgcatcgtgctgat, gggtcatcctttgctt, ctatatgttgatctag, agaactatgctggtga,
gtaagaggcagtgtga, aagcaggcctggtgcg, caagaaggtttttaca, tagcaccatcctctat,
gccaaaagtatgtccc, ctccaatagggggagg, cttctttgtcgaggaa, aactgagtttctcccg,
tacttagaaggcagta, aaatgagataggtctc, aaggaacatattgtag, ttttcgtgagaaatta,
agtagcattatacatc, gatttcaacgtaaata, taagtaactctagtca, aggcagacacgaatag,
ccaggataacacctta, ctttaaagtttagatg, atgttttttagctgac, gtgttgttaatttgtc,
aatgataacgctaaag, actatgacaagcccaa, taataaatggggttgc, cccgcttaggctggac,
agctctgtgacatctt, aaagttattaaccctt, cagcaatatttgtccc, caggtatcccccagcc,
cccatacagcttacca, ccgatgaaaggagcat, tgtcctttattagtta, ctctcattcctcgccg,
gcaggaccaaaagtca, ggagacggggggggtt, aaagatgggactttac, gtaccgtctgccccca,
aaatcattttccgaac, atcccatacctcactt, cagtttagcccagatg, tcgctaagctttatgg,
attgcgagatcttgca, cagtgtgagcgactca, aggaagaaggtgctag, ggtgcttgggtgcagg,
gtcccttaaaagctca, cgtattaatgaggcaa, actgtgcagacgctct, aaccctgttacctgca,
ctctcactgtgaagaa, gctggtttttttatca, tgacacagtctcctcc, aacggggattgtcaca,
attcatttctacgtat, caatagcttgattgta, agtggatacatctttt, agacctcaactaatct,
tctagatagcaactcc, ccaagccatgccccgt, ctgtgttaatggggca, gggctactgagttaga,
aagcctccatactttc, gcaataattaaactac, atgagacagctattta, gagccctggagcaaac,
attcgattaaagtagg, ccgactccccagttct, ttggccgacagaagat, ctcccccatgcatttg,
cataggaggtacccca, tgaatctgattgttag, cagactccggcaccat, tgtagttttcgtaaag,
aaacctttctgagccg, ggtgtccaatagaaca, ccgcggtccccgtctg, catggatagaatatgg,
agtccggttcacttgg, atatgcagctggcttg, agtggatgacttggcc, ctacccggaaggctta,
ctctattacggataat, taagtttcagtatagg, ttatcgacaaggggca, tgtagttggccctgtt,
gctgcctttccccccc, gggtgacagagtacca, gagcatacgaagttca, ctcctctcgacttcta,
ttttagtagcgatgct, taccttagggcattgc, gagatagccaaatttt, caagcatgttagacag,
atctcgaagctctcac, tttgctatatccatta, tccgccccccgccagc, aaaacggggggatgtt,
ctctgtgttgttacct, cgctaccggccttgcc, agctgggatcacatat, taaaccccaattttat,
acctgaaggcacttac, agtatggctaatagtg, tacttcctacttgacg, gtgagcccgggggggg,
taaaggatctaccttg, ggatgttagctataag, ggcaatcagcttggca, atcatcgtaattctca,
aggctttacttattcc, tgacgagcttagagaa, gccctcttagagacga, tatggtaagctcacta,
acgttcctatgtttat, ggagtgatagcagtgt, cctttgataccccaga, tagttcttttttcagc,
atcacaaatttctgac, gtatggacatggatta, gccttagacggtgacc, tcgatagctacctacc,
gaggacattagattta, ttacccgccccccccg, ctgcataaagatctcc, tggcccctctaaccta,
gattgcatctcaatag, tatagcccagcatggg, aaataccaacttcgct, gaccgagttagactct,
aagcacactagtacac, acacttccgcatttat, agagacctctattgta, tgacggggctgagctc,
cttgaatgtatcaggg, tgtcagtcgcccaaat, gtggtctttaattttg, gttacttaacacatcc,
aataatgcccaccgtg, atcaccatcgtgttac, aagcttattgacaaat, gaattcaatacgtttc,
taaccggggcccatag, tcatcaataatcctat, aaatatcgatatacat, tcggtttcctggatgt,
tagacgtatagacata, caaggcaaccttctcg, accgctcaagctgtta, aagctacgccttctgg,
acgggcgtgggattac, tggggggggggctatg, ttatgcagtctttgtg, tgtgcgtctgcagtta,
acgtaccatgatgttt, caggacggcccgcgct, tattggaaaagctcct, ttcgaaatcatgcttt,
gatcagcacggggcac, ccaagcctacagtact, gaaagtaatggcaagt, ctgggtcacatggaac,
cagttacaccatatgc, cacaccgccacccgga, cgaggcttccacacag, ctctcaggtcccgggc,
tccccctgaacccatt, gttagccacgacaccc, tctataacttatagtg, ggtggattcatattgg,
cgtagcccggctatgc, ctccccagtggcgcga, atgactggactcgaga, cccgtgggtatggtcc,
agtctgaaacgagctg, aaaaatttcgagtctc, ggacatagtggtagca, tgtttgtggggagtgt,
atgcttgatacgctgt, agcaagcacactagta, agcagatgctggatag, tatgcccagcccccgc,
tcctaagtaacaattc, gcaaataacccccctt, ttctaccatgtattgg, ctgagctttttttatc,
atagacatgtcaattg, cgagctttttaaaagc, gtatatgggggggtct, tgtaaacgaacagaga,
aatatgcatttctcgg, gtagcttctcagacta, tgcagctagtgacctt, tttactctacctacga,
acggggggtaaatatt, taaaatgatcacgatc, tttacctccacgtgac, tttagcaccaatgcga,
tagaacgcacaactta, tttttgaccccatatt, tgtggcagatatgtac, ggtgggcgaataatga,
accagcggtggggcta, cagtcacatagggggc, tttctgttaagttagg, gggacatcagtttctc,
aaagccataatcgtgc, ctgatggcaggatagt, tgattatgcaaggcgg, atatgaggctgataca,
gagtcttactgattgc, catctttcttcgacct, tgatctgattggtact, cttacccaactagcgt,
gataaatacttgaacc, attgtgcgtactatat, agcattcacatcctga, acttatagcatggatg,
aggaggcagcttaaga, caggttatgtcacgca, atgcctactctcgtgc, tccaagaaaacggagc,
tatggttagaccaaca, tatggttaaacccccg, aaagggatottactag, taagggaaacggggga,
aaaacatccagccgta, gctcttggtggagatc, gagatagcccttaatc, acctttcctcatccta,
cagagctcgtctcctc, tgtcggaccaactatg, gcgttgcgcacttccg, gttccaaatggataga,
cctcgatgagaggcct, gagagtgaatactccc, tggttatgacccctga, ttctatgtccagogtt,
aatcaggcagttgtgg, cctatgccgacactca, tactcatctcctcgag, tccctgctaaaatggt,
ggtaacagacaagcta, tcttacaccttagtcc, ggtgggatgtgggcat, ggctgcgaggcttcca,
ttatacgaattgttaa, gcttttttttggcgca, ctttatagcagttcct, accactatccccccca,
gctccgatgaataaga, aaccggttaaaaaata, cattgtaggagtgttc, ctgatccgctgtacga,
atgaggtccacatgat, caaatgtcctgctagc, cccgccttagctggta, ctgcctggatactgca,
attgtaaggggttttt, ccttgacacttaggct, ctcgattttaaaacca, cctctattcttcaagc,
ctacatagagggagaa, acggtcacctctgtcc, gtccccttttttttag, gagtgttatccaatag,
aacgtccgtcagcgtg, agagttgatagcatga, tttaaacgtctccata, agagataaagtttaac,
gatggtggttgtttcg, ttctccccccctaaag, cagatccttataagaa, gagcacaaaaaaactg,
aaaaaaatattcgagg, agattctcaggttagc, tgctccaagaccgact, tatgttacctatctta,
gtgagcaaataagcag, ttttactctacctacg, tcaagctgcatgttta, gattccagtgggtgtg,
ccccgctgtcttcacc, tattgtatcgtgaata, accttatgaactctag, tgtcagcgtgtggctt,
gttctagagatccaag, tactgttttttagtgt, acagtttcagttcgcc, atgctgtgggcgaaca,
aggcttatgaagaagc, gcccctcgacgccttt, gtccggcgcgggctaa, agcctcgaaatctcag,
tatgggggggaacaat, agctagtaaggcagtg, cccatggcacttatat, aagaactcagccataa,
gtcaccactactgagg, ccacttcacactttgc, aggtgaatgtgtctag, ctgatgggggaaaaat,
ttcctacaatgaggta, tatagtaatcctaatg, atgtcacccccccgac, atctgtcaaatccata,
ttgctgcgcagctgtc, ttcgtcttctcattcg, tgataaaaaatgaacg, ccctttttttgggctt,
gatgtactaaaaaaac, taagcttggactaaat, gaatgtactcctttag, ctatgttctgtaatcg,
taattgttatgtttgc, cagattattcatggac, cttagggtagcagctg, gcactgcagaacacat,
aaagtaatagtgagtt, aaggattgagccggct, acaacagccttttact, agcatcaacagctagg,
gcgtaggcttatggga, cctttttttagagacc, tagaccttccggatgg, tgcctttttccggcta,
tggtcctccttagtca, tcaccaatttagccag, tcggataagacgctga, ttgctttcactctcgt,
gtggggggggggtact, actcaccacgaaggta, cctctgtggtctagta, ttccccccccatatgc,
atcatgtttgagaact, cttggtctgtcattac, ggtctgagaagaatac, tctggaaattacagcc,
tctattcaattgtatc, agaaagttccttgctc, gatgtatggatggact, atatttatgttcgtgg,
tccacggctcagtgcc, gaatctcgtgaaccct, cgctaaaaaaatctgt, tagagattgtgacgtt,
ggtcaaaaaaagatgt, gctgttagtcatcctt, gtatatcaggtcaccc, cgctgcgcgctggggc,
gctaagcatcctggtg, ctttaattgaggttga, ccaatgtaaaaaaagt, tatcctttaaaattgg,
tcgggggggggcattc, ggtgatcataacggca, gtggttacacacgcct, gaggggagctggatag,
cgacgcccggccccga, ttacccctggacctta, gtgcgagaaggcagaa, tatggtccccgccgag,
cagttatttacctccc, taaacgataaagtgaa, ctgtaacctgaagtta, cttcaatgccgcttca,
tcagctttgccctatg, cgattcccttggctgg, gaccaccacttgatca, taacagctcggttaat,
cgttactaatggagct, ttaatagctaatcagc, caatctgacgtttttg, ctgactctttaggtac,
gtgtttgttgcgtgtg, gccacttagatttcat, ccctatgttgaaactt, agcttttgtaatctct,
ccgggcctgtgtgcat, tggcataagagtaagc, tgcacattatgcactt, tcactcattaagctta,
tgtgagttgtaagatt, cggactgtacatgtat, ccttagctcttggccg, tgttacccctcatctg,
tgatgttcattagaat, tggtactaggggaggg, atcacaggggggagct, ttggagcatacgaagt,
gcaacctattaaagaa, gtggcagtcgccagta, cttcccggccaggctt, ggccgctcggaaatca,
tctatttgcctcatgt, attctcgcaccattta, agaccctgccttaggc, taacatgataggctct,
ttttttcgaaattagg, ctcattctgttaaaca, gcgaggctggggcttc, ttagcgcctgggaggc,
catgaatgctgtgact, ttacacttatcaaaca, ggataaaaactagtgc, aggcgctcgagtacga,
ctaatgcaaggggggt, tgggatccactttccg, ttaaaccccgttctac, cgctttccatcgttcc,
tggcacttaagtggtc, cctcaagcgctgcgcg, tgagcaacaccgttag, gtcacatgtagataga,
tcttgcaaggtataca, tttctattcaagatcc, cagaagtttggcgtgc, gagaagcttactgatc,
caacagctaggcgggg, gggcccacaggcaacg, gtttcccccccccaac, gggaatgcacgccgta,
tttcgttggtattttt, ggtatgactccttatt, tacatccaaaaaatag, tatgagacagatggat,
ccagtcagatttaggc, tacttgtgaggagtct, aatatagcctggcata, tggctggttcaaatag,
acaaggcccacgaaag, taggtgtaagctactc, tcaggattcagatgac, gcctagtaatctcaca,
cacggtaaccgattag, cagagatgtcttatag, tcactggtaggcatca, ttgagcccgataggcg,
tgctcagttcccttga, ctgtgtatgtcttgtc, ttactattgactgtta, attaagagttgaagtg,
ttgagccagtaaggtt, ggaatgatgtcccgac, tatttaacgcaattat, aacttaggatcagtat,
ctcggaaggctgtcat, gcctgacgcttctgtt, cggtccccgtctgtga, tcgttaaaaaatggaa,
cggggattcgtgctcc, catgtaacataaggct, ttgtcagtgagtacag, gcttagtggcgagcgc,
cctgtccagtcagcaa, cgcatggtgcgaggga, tgatgacatgcttcgg, gaatccagcataacat,
cagacaaaaaaaacgt, tacattgacagcagta, ctttattcgattctta, atctgacctgcctgtc,
actaaccatcctttac, acataccttctgctgt, gctgctaggccagttt, tgtgctcctgacatta,
ccagtctactgactct, gaacagatgatctatg, caaaccatatgtgctg, tctaagaaaacgcctt,
tcatacatttaatctg, ctgcttcattaaccaa, tcagtcaaccctagga, gtagcttagagcggtt,
cttctgtctacatcag, acgctcaggaaatcga, tagtacatcaaatata, atatgattgttagggg,
ggtaaaaaaaatgcga, catgcccaaaaatcag, cggggatcttcagaga, acttttggttcactgt,
ggtgtcgcatgcctac, ggaatgatgcaatgct, gccattaccttcagcc, gtcctcatttaggggc,
ggcgatgctgggctcg, cagtattctgctagtg, actgttctcacccctt, ctgtaacacttatcgt,
agctagcggagatcgt, cctgctatgctctcat, gtgtgatacagttaga, tgagacatgtatgcag,
taaaaaatcgtgacta, cggcagctaggctgcg, ccccttgcgcttggct, ctttcttgttagagtc,
gcgcactgctggatgg, attcgcgtctgtgtga, tctcctagacaactta, ggcttagcgccgcgca,
actacaactccgaaaa, tcagcgtgtggcttaa, ctggtcaccatatatg, ttggtcaacgcaattt,
taccctgaacctcatt, aagatccatctaggcc, acgttgaacctttttc, tcagtccattatttgt,
gggcggttttattatt, aggagttatccactgt, taggcatttcctgaga, tgaacattagtgtgga,
gggttttcatatgtag, ggtatgactcatgagt, tactaacctccagcca, tatcctttttgcagaa,
cataatggcagcctcc, attcagtaaccttgta, tcgtatacatatagga, tctcgcaaaaaaaatc,
tggaccgatttgctta, tatagctccccccccc, ggttggggctgtttta, ggttgaggttttggac,
ccggcccctccatggc, ccctgttcccgcccgt, agtgatcatctattag, tggggactctttaaaa,
ctaaccttgttatccg, ctgaggcacttagaac, tggaaaactcgtaagc, aggttgtccacaaaga,
tgaaggtaaggatatg, ctctatttctcgctgg, gcttttttttaacgac, gaagtgtgtccggaac,
gttactaatggagctg, tagtgagttgtaccta, tccgcctgcatcagtc, atcccactaacagcca,
gcccgccgcccttcca, gactaatacccatctg, aagtattagaatccta, tcggttgtcttcagcc,
acacctacatctgact, ggctgcggtggctacg, gggtgtggcccccccc, aaggcatggtcttatg,
ctcggaaacgcggggt, tcgggttgtatagtac, gcttttctcaagcgtc, tgttcccctccgtatg,
catctgatgaaacgga, atcccggtgttcgggt, gccggtgttcttcagg, actcactgagatgaga,
aagtggggtatattag, agcatacaggaccatc, aggaagactagggcgg, tgagacttaggagtga,
cactcccaacgtcaca, atggctggtgtgccga, tcgactctcacttcta, cgttcagattgactct,
ccggctcctgcaattg, attgataaagcggaag, gtcaatttaaaaccga, ctgagtctcggtcgcc,
agccagaaaggactta, gcttagtcttaacgga, ggatagagttgagacc, gttgttgcacttaagg,
gactcttgtttagcac, aggtcatgagtgcgag, ctctgctgcagaggat, ggggctccacggctca,
tccttgtgagccgctt, acggggctgagctcac, ctcatgcagtaagcct, aacgcagttttcatat,
atcgatgtgggttttg, gctgcctctaatgacc, atcttaaaaaaaacgc, ggggccccaaggcttt,
tacaaaatgacaacct, ttaggtaacataactg, agaaaaaaaacggcct, acacttaccataatac,
cgcattttttttagcc, gtgggggggggatcca, ttagaagagggttgag, gaatatgttctgtagg,
acgtaatagaacatct, tctgaattgctgggcc, acctcatggattataa, gcctgcacagctcgct,
tagctgagggggggcc, gtgcattttccatgat, atttgagatccccaat, ttatcttacgggctga,
caagggtcaatagaga, gacgccttgcgttcgc, cagcttatcttaaata, tgtaacccagctgtgc,
gggctgaaaaccgctt, ctttacacacactgta, tccaccaccaacccta, gggttgatgtctcatt,
gcttccccgcttagcg, atcacgtccttgcact, cgcttaggctggacgt, ctaggtgtgatccact,
gtctaaagtagcttat, actctacaatccgaaa, tgttctttacacagac, ttgcgtcttcgtagat,
atttgtgacctagagt, cgtagttagatgaaac, ctttaaacagcctact, agagaagctaaaaact,
agcaaaaaaaacacga, caagtgtgtttgcagc, gggcttaggatcctct, tctatgagctttcact,
gttctgagataagact, agacaattgatcaaca, ttatgaagtagagcca, agtcacttactgcagc,
tgctgaaacgccacaa, ctcccccattgattaa, tcattagaatcgcttt, cgggcgctgcgcgctg,
tcagccctcttagtcc, tatatagattccttta, gttattgtccactcca, caatccaccccttctg,
ccgtgccatgtcctac, cagagaaaaacccctc, gagctactaacctgta, cgggtggaggaggtga,
tgactctgcccgatcg, cgccattttccacccg, ccactctcacaggtaa, ccacttatcccctgag,
tcatctcctcgagttt, taaaagtctcaaggtg, gcatagtaaagaagtg, ttgaagctcagccaat,
catagcagagcgagaa, actgtatacactttct, gcttcagtctgtttag, atgtacacgccccttg,
aggcgtggtgtctact, ttggacgtgttgaggt, cctttgggtatctgcc, tatctcttgtggggcc,
actctttcatagccaa, agagaacttcctctgt, tgttttcacataaacc, atgaccgtagttgtgc,
tggtggactgtggacg, ccggggatgaccgtag, gttatatcttacatgt, attgccgctcacgtag,
atggatggcctgttcc, tgattcttccaccggc, aatcaacgacttgtga, ccccgaggtatcaagc,
caatcaactcattgaa, ttagtatttgcacgtc, acaggtccctatatta, gttcccccccttagta,
acctgcctatatgtaa, tctccccattaatgag, ccggggggctgagcct, gcatgtggcccctaag,
acaacactcacgtgat, gcactcttcttaaccc, gccgcatcccgctctg, agtttggcgtgcacta,
gtaaataggggatagg, ggtgaagcttagagct, gcttcttccgccacta, cgccggctaggccgcc,
agggactataagcgtg, ggaaaactcgtaagca, gcgcggtgtcgcacgc, tctgtgccccccgaaa,
acaagacgggccagcc, acgtctactctagtct, acacttaaaataaagc, ggccgaaaaggaaact,
acagcctcttttttac, gacttgaggcaccgcg, cctcagattccgcagg, tgttttaaagcgtgta,
aggcttaggcggaagg, tatacatcaaaagtag, gcctccgctctcaccg, tcaagatggggcagac,
atattaggcaggatga, ctgcgtccctgcctgt, ttttagaggcttaggt, cccatatgggggggga,
ccttgcattttggact, acctgaggacttagct, ggaccagctttgtttc, ctgcgcgctggggcgt,
ttagggggggtgaaag, caccctccacgacgcc, ggagactgtaacagct, ttatcagcacttagtc,
agggttaggcggaaga, aactcgtgctccgccc, cctagtaacaaatgtg, cacggggggtgcagtg,
ccaggacatatttgcc, tgcctatacagagctg, ccaagattgactgata, aaccatccggtgatgg,
ctgaccagcatggtta, tcttggctcttagcat, gaccggggtctcgctt, accattttttttacca,
gttgatcttgcgacca, agattatggcgtctgc, ttgcgagatcttgcat, gacactttttttagcc,
cgcgccctgcctgagc, atggggggtatactat, atagacgtatagacat, tctaaaagcgttcctt,
cagtaaactaccgtaa, tgtaactatgtggtca, accaaggtagaggcaa, tttgccttagctttag,
ctacagtttgttaggt, ctgatacaagttggct, attcggccagtcctct, cgtgatataattgcaa,
tcccgctttccatcgt, gttgggttcaatgaat, ggaagccacttataga, agaagcttcccccccc,
agtctcactaccctta, agcttctacttttagg, aagaatctgttggtat, atctatattagtaagg,
ggctggatgatatgga, tatatgccgaaaaaaa, aatatcaagctaagct, cctgtttcgagggctc,
ctcctttggacaaatg, ctgtattcacacttct, aataaccacccatgta, gcgctgagcgccgatc,
ccctgcagtatggggc, gggaactcagaattat, ttgtatagtactgcgg, ttatggggaagtatgg,
agcttgcggacaaccc, ctgcgtggcagaggag, attccacgagtttttt, atgtccattcaagtca,
acgtgttgaggtccag, gcagattttttttccg, actggatcaggtcagc, caattcttgcgtcttc,
agctttggtagtagat, tatcattcctgccgga, actattttttttgtca, ctatcctcacccccca,
ctaaattaccctaggt, cacccgctgaaggatt, gtgaggattatgctag, tcacaagcctttaccc,
ggacatccgccgctgt, tccggcgcgggctaag, ccataagctcctccct, aagttcccacacctcc,
attgtagtcactaatc, ccctggtagaagcaat, gcttagacgatgggat, aatcctcccaaataca,
gaaagtattagatgct, gcacattaaaacgttt, ctgtgcggtctgggtt, tcttcgtagatccagc,
cccctggcttatocca, ctaggcggggcttcga, gagcttactgagagcg, atggtttagagctgga,
aaataaacttgccgcc, aatgcttctattacac, ctccctatgttgaaac, tccactacaggcagcg,
tgatcgacccacctgg, ggtcggggggctgacg, ttaggttggagaatcg, accttggcctgcggga,
ggctggaatttcctcg, cttgtggtcttatgta, gtatcatttacgcctt, tagggggggatgaggg,
ctaaggcctactcagt, gtcactttagcgacct, tcgtggcctaaaatcc, aaaggtgctgagcagc,
ctgaagctttgttaga, tatccggcatgtagaa, gtttacctgaaattgc, ttacataatgtaccga,
tatcccccccccgaaa, cattagcaaggccttc, taaacctgttatgact, aaagggtccccccctt,
ttaaatacccccccca, accaactgcaactatg, caagataagagccccc, tttgatgttaccctta,
gctaatgctcttagga, aggagggtctagcctt, gcgctcgagtacgagc, atcgtttagcacaacc,
cttgagcatctcccgc, catactcccctattct, tatggaatgatgcaat, ccccccccaggccata,
accccactagaggcat, gcccctcttaccacca, acgtttggggctctta, aatgaacgtgaagaca,
catgtcattaatgaag, atctttatttaagagc, tactccatggtggtgt, aaaaagcgttcagaat,
gacatccaccaacaag, actcccccccccgcat, ttttcaggagttgtca, atttgcgcagaaattc,
ttcctcttccgatctg, agtcagagcctttcag, ctcattgattaacaac, tccttggtaatagttg,
aatttcgagtctcctt, acttgtaactatgtgg, ccgggcgctaggcggc, ttacagtggacatacc,
cgggagagtccggggg, aaagccatcttctccg, tggggggggttgtatg, cccttcactggttatg,
tgtgttttaaagcgtg, ctctgtcaccccgagg, aagccaacaaccctac, ctgacagcgtgtaaca,
atgaatatcttagtgt, tgatacatccggagtg, ttaggggggggggggg, gcaatttgtggggagg,
atgtgtcagagatcag, tcacctcagcgcttct, atggagcttgcaaatc, aagggtcccccccttt,
actctgcgatgcatct, tgagtgaagtgctaaa, gtccccttgcccacat, cttgtcccccccgaaa,
gggggccgggggagat, ctcgcaactgcactct, tgagagaactacttta, tgcttccccccccatt,
ttggtaggcttcaaaa, tggggggggtacattt, tcctcgtcctggtgtg, acgcttttttttggcg,
acggggggggaaaagg, ctggctgtaaaaggtg, gtctatttgcttagag, taaacttccattttgc,
cctaagtgagttctat, ggctcctggtgacccg, taagacttagagtata, tgtgctcgccggctgt,
ctttctacgtctgcat, gccttagtgatgtgcc, atttcactctcgtctg, gtagaccatctgttga,
ttttttgtaccccgat, gaatagccttctgcca, gatgcatttttttcga, atctaggacgtgagga,
acaccgcatttttccc, ctaagattcccttgcg, tgtcttgttcacttag, cgcacaatagcagtgg,
gcagcggatcctttct, gctcgcacccattcca, ccaggtcggccaggtg, atttagcccagttata,
atgctcggttttttta, cagctaacaacgagat, tcaccctgcacttgtc, tgtcctgtaaggtgtt,
actatccctttaacta, actccatatagtttcc, gcaggaaatttgcgca, gttccagcaaatggcg,
cagaggaaccccccac, ttatgacatccccgac, ctcggttggtttgtgg, gaagcaagcccttaat,
tcaggtaatgtaccca, gccaatataggtaaca, ggggttgagggcgtag, ttagcctgatgcgatg,
gatccaacactgttct, attgtgtaattttcgt, gcacctccttctatac, caaccagaatctacta,
gaccccaggctttgac, actcctgaaaaaaagc, cctatgctagggaagt, ttaaaatataaccgag,
gaccgggtgtggaggc, tgaacaattggccact, attcccgaattcagtg, atctatgccacgaagt,
ccccctaccccggcga, tgcttactacatccag, aatagcaaattcgaca, tccttagctcaagcgc,
gcttttttttgcttac, agtgaagccccgatag, gtgttttttagggggc, cggacttaacccctac,
atacattgtcatgggt, gcaaatcaatgcaggg, tatcacaatcacttaa, ttccccccccagcaac,
gctgacctcatgtgca, tactgacgatattaat, tgcccaggtgggatta, agccagtaggcatttg,
cagggcttagtgctcc, gctaatgactaaatat, gaggctgtatgcttta, gctcacggaaaactcc,
ttgctcgcccgccccc, ggcgcctccaatgtgt, tatgcattctgttgtg, tcaagatacacttaac,
aatggtgcctacctgt, atgattgttccaccgc, gggatccactttccgg, ctccccttagtaagtc,
ccacaccgccacccgg, acttatgtaaagatgg, ctgttgcaggccacgc, tttcgttatctgtggt,
cgacgccggagccgtt, ggataaactgtaccct, tgtccagttttggcac, cgtccatggaactagc,
cccgctctagaagggg, gttcgaaatggaatct, cttctgtataggtatg, tgacttaacgccacat,
cgaaaaaaaatgagat, acttgctcaattcctt, gtcagggatcgtctgg, cagcgacacgagcgaa,
ctcttgtcgaccaggt, ccaaaaaaccccgaag, taatcccctctactcg, attttctcgcctatat,
ccggatagggggaggc, ctccgagccaagtatg, aggacccgccgctccc, tacacttatgcatggt,
aacacgaataaatcaa, agccgagatcgttaca, gtgtagtagtagggag, gttatgtttttttagg,
tcttagtgatatacgt, taactttgcttagtac, ccttagttcatgtata, aaggcagttatcgaca,
gtttgagaagtcgaaa, gatagctctcagtgcc, cccgaacacttggtgg, taacaattatctgcct,
acccaaagaggctatt, acctgctatgcagtct, attgacaccccccgcc, catggacaaggccctt,
ctaaactatgaaggtg, tggggaagctcctaca, tgggtatggtccccgc, tcgccctcagagcagg,
tattaaaatgcgcact, gggcatttttttgaac, tccaacatcacacatg, agattagtgttgaata,
atcacaaaaagaacgg, tcctgactatctagct, gcctgagtaggtctct, tcaaggaagcaagacg,
ggtaaattgtacccct, gtgagcaccactccaa, gaatactacatgttta, actacaactactcgcc,
cccacgacagactctg, ggtagcctcctcatgc, aatggagatagagctc, gttaaaatcccccccc,
ctttgtaaagtacgta, agggctgtcaagccca, atttactaatatgtgc, gtgcacccccccagcc,
aggagatcccttatag, atatctacccccccca, gaggtacgaaaaaaca, ggggttcgtcttctca,
ggttttttttaggccc, tatgttggtcttacca, gagcaaacggccaaat, aatccgcccgcctgag,
ctgagttgacacggtg, gttcaaaaaaaatccg, cacctcagcgcttctc, atggcagcggagggtg,
aggcagttatcgacaa, gtttaccagtattgat, cccgaggtacctcggg, aaacacggttttttaa,
tcgggatgctgcggca, cttcccgcttaggctg, agggaaacgtatgaca, ttatctgaggcagccc,
gaccatcatctgtatt, aggccttaaatatatc, cggggccttagggaac, taattcaggtatatga,
gagtactccttacttg, agagatcctccattag, ttgccaggcttacatt, gctagcggagatcgtg,
gcctttggcagcttcg, aagctgtgcgtcctcc, tatactcgaatcaata, ttacaccctcttaagg,
ttagcagacgaaagaa, tcagcgccccgtccgg, ggtcacggccggggta, agttgtcctgctcctt,
agggttatctgtctga, atggctagaccccgtt, tgacccccccaccttt, cgtacagtggcttgat,
ttctatgaatgggagt, tccgaatgccagcaga, gctagctactcttata, gcgctgggttcttgat,
gcctttttccttaccc, gtaggtgaggagcatc, tgccgcatgggcttct, ccagtgtagagattgc,
ccttagctactcagac, atgacgggcgtgggat, cagcgcattaccgtgt, gaggcttaagcgggaa,
actaagtcacttgaga, gttataccttcctccc, gaattgtatccactga, aataaacagtcaaagt,
ttcttacgggcttagt, ctcctcttatctgttc, gctgtagagaatgcat, cccctagcctaaaaaa,
gaggcatgctgagggg, acgtagacattaagaa, ataacccttctcacca, gggacatatgcacagg,
ctccatctgtggatag, cccggggtgcatcgtg, agctgtccgggtgagg, tagcctgatgcgatgg,
aatatgtcatttaccc, gcagtacttaggaaca, ctcttatgagtggtga, tgtagtggcgcactct,
tccctctgcggaagga, acctgggcacacttac, ttggggggggagtgca, tcatagctgagctaag,
aaagttatagcaatac, ccggccttgctttacg, ctagtaggcaaccgtg, gagtgttttttaagac,
cagtcgcttttttttc, cagttatccggtagag, cgacacggcttctagc, cggatggtctcgatgt,
atataccttagagggg, ctgtaaaaaaactatc, aacccccccccacaat, tcaccaggctggctag,
tgaacatgagctttcc, catccactgcttctag, gattatggatcagtgc, gggaggcgtaggttcc,
cctcgacgcctttttt, agagcatgccggacag, tggtgttccttaaagt, tggtatacgatacaat,
ttgcttacccatctga, cctgtgcttcttaggt, aaaaagtctgtagatc, tcaaaaaaaaggggcc,
tacctggatgggcgcg, agactttaatctgata, aataaagagctacccg, cagtttcgtcttatcg,
tcccatgtagtaccac, ctatcactccccccta, cccctgtagagtacac, gaatacgtttgtggat,
gccaatgatgctcatt, gtgtcttccggaggcc, aaaaaacgggaaatgt, ccagtgcgactgtgtc,
acccaaaaaaatagtg, ccgtgggtatggtccc, ctggcatagtaaggct, ggtgggatattccctc,
aaagaggcgtatttta, aattaataacagaacg, gtttaagatgactaca, gcactgtgaatctttc,
tgtgcaaattcgtgat, gagtgatgtttcctag, tcactggtgatgtgtc, cccgggcattgtggct,
cagtcgtccaagtagc, ctacagatttccacca, aaactgggaaacgaac, ttggtattttttttcg,
gtggctgcttgtctct, gctaaaccccatctat, cgagatggctccacgg, gattacagacgttaga,
aagttagcccctgacc, ttacactgttcgggca, ttattgagtctcgagt, actgtcactccggtga,
taaatcgtgtgctgtg, ctgtcggtgagtggag, cccgagccttgcgagg, aacaggaagatcgttt,
tcacacccaggtacat, aagaagcatgtccaat, ccactgaaggcccatt, tcacctgcgtctgtga,
ctccttatggcagtgt, aacctcatttgctacc, ttggtgagtctgggat, tgggaacctaagattg,
ctagctgcggcctgcg, gccccccccactcagt, acgtaactgttcagcc, gataaaaactagtgca,
gaggtacgggcggatg, ctaggagagactccct, tgggcttggggggggg, acagtatgtggatatc,
ggaggctgcggcatgt, tccaatacaggtagag, cacaggtacaagggct, aacaccgtcttatctg,
tcggaaatcataagtc, gaacagtgccagtgcg, tgatgatgtttcccta, atctgaatattccaac,
tattgcatgtaggggt, cccatctaggacgtga, gtccagcgcctactta, cccccctgtgccccct,
ggttggattatcaatt, tgcgagggttacaggt, ggcccaaataatgact, aagccatattggaatc,
atacatccggagtgag, tagcgttattgtcatc, ttagaacacgttttat, ttgcgttcgcccaaca,
gctaaggtcacggcac, atatctgcctgcaggt, cctgactctttgccat, aacgaaaaaaaagctc,
taatgcagcaattatc, atgtgaaaaaacgtaa, atttgcataacaaacg, tacatgcgtatggtgt,
ttactaatatgtgctt, cacgtttatcgtatga, gtttttaacgtgccgg, cgacaggggcccacca,
tgtgcatcccacccta, gtcctgtgcagctagg, tctatcaccccccctg, tttaacgcattttatt,
acgagcttttttttag, tcacattggcctcgca, atcaccagataatcat, ggctctcagccaaatt,
aaggtgagatacgaga, aatccccccagtgccc, actgtcggctattggc, gtctcaaactgtagct,
ctactctgtatgaggt, cccttaaggtgaataa, cccttgccgcagcact, gcttttttaagaattc,
gccgtggaccctctcg, aagtacctctgtgtaa, ttgatctcttcgttct, atgttagttgtgttgc,
cctgcgataaatgtta, gaaaaaacggacccag, tccatggacacttcgt, cctaggtgcttgaaat,
tggaaaatcctaagtt, aactccatctcgcaaa, cggatgacctcgtggt, ttgagcacgggggagc,
ggaggagttctcgcgt, taccctcgcttctgcc, acttagcctaggtgcg, acagatggctccctgt,
gctgagggttattatg, gaacataagcctagta, gctaaaaaaaacgtcc, agtcccggatgtatac,
agaatccctggcagta, gtgcctttttccggct, cggaggcctagggctg, ctcaggacctcacggg,
gcaatgggggggggag, ccagttctatgttctg, gtcacgtaaatgtgga, cacttatttttttcgt,
agaactggacatccgc, tagtctttatagtgtt, gatagcgtagctcgaa, atgtggccccccttac,
ttgtgagaccataatg, ggcggtcttcagaacc, aggatccccatgacca, gtgggcgctcttagtc,
gaaaaggaatgacgct, ataagacggcattttg, agttaactaatggcac, gacgctcaaaaatgat,
tggcttctaccgaggc, gcatcttaatgtgggg, gagagcaggctagatg, actccacggagagcat,
agtcgttgctgtcttg, aacggcaaggggggat, gagtaagcttacccat, gagagctttttaatgg,
tgttttttaggggggg, caccggctggaactgc, tgtgtaataacaacgt, tagtgttactaagctg,
tctccccccctacact, ttataaggtaatattg, atcccatctaggacgt, ggatgccctgtctgac,
gtgtcacgttcctttg, agccgtaggaggaaat, aatatgtgaggggggg, tcttctgcatcgtagt,
gaagagttcaattcta, ctgcgaggcttccaca, tgtagaatatcttccc, ccagcggtggggctag,
gacactcttttttgca, gctctatttctcgctg, atcgattcccttggct, gcagatggccaacacg,
aactctcaggtcccgg, cctgtcacagagtgaa, aactccagattcgcca, cgctatgcttttttta,
tcatagggtgggtcag, gtgttataaggatgag, tgaagagttccggttc, tgctactgtccacaag,
ccgaatacagaacttt, gggtgcaccctaatgg, agaccgagggcactag, ctcacataacctaacc,
gaaagttacaggaggg, ataacatgctattagt, tactcgcagttaggga, acatctggcttgttac,
ccggagtagacaaata, tatgactctcggggtt, gaacctgggagactta, aaacactcaaatttac,
agttctcgcgtgatgg, ccaatttggccctctg, ccatgcgctcagcaca, tctgtgtccagtagtt,
tcttagtagctagcat, ctctggtgtccccctt, aagttgcccctgtgat, cgaaaatttaaagaag,
cgcatccatggatacc, gaaatttggtgtatgg, atactatatggaaatc, aattagcgagttgaat,
ggaagagtcgctcctc, gcaaatagcactcgtg, acctggatgtcaatgt, tggccctccaaggcat,
gcaatcccggcccctt, gtgtttgaccaggaca, gagggcgtagaaggcg, cccacataatgttctt,
acttttacctcacgta, ccactcaggcttagga, aggcttaggtggacaa, gcgtcttcttactaat,
ccgtgtatcccggggg, tctccatttagatacg, cctattgaaaattaag, gttcttggacctgaag,
ctttgcagctataaca, tgccccccctctcttt, agcaccctatagctta, tgaggctgcttaaatc,
ccctactaaataatac, caccaaactttgctac, ctctagcatataactg, ctcttgtctgggataa,
cctttgtggatttgtc, ttagttttataggggt, ctcccttgattagtct, cacatcagaggataat,
gtgacataccatccca, gtgaagacttttagta, aaatgttgacctcccc, tgggggggggagattc,
caatctagcaaaactg, ggctcactttagtgta, ttgtgagaccaatccc, gcagatttcgcatcag,
agttcatcccgcacca, ttcataagggctcacc, aacgttaaagatgatc, tgggagcctttaacct,
tgttaggttatggctt, caaagggacacagtgt, ctgatgggctctatgg, gccagcgcattaccgt,
ccctccaggtccgtca, tacgtcattttgaatt, atgcgaaaagaatgac, gaaaaaacggggcata,
acaatctcatcccaca, agtaagggcagttaca, ttagccccctctccca, tgccctccaggtccgt,
gtcctctcttagagag, aggacttgcttctaag, tatctccaacgtttct, cctcgtgggcctcccg,
gactttccttccacca, attacttcttcgtatc, attgtagggggggacg, atatgcgatgctttat,
cagtctcgaacaaaga, acagttagacctgtct, gctgtctggttgttag, agtttttttactggag,
agttaattgagaggtt, actaatgagtcaattg, aatagattccattgag, tgtgtggggggtattg,
tcaatacagattaaag, ttccggttctgacagg, ttcgtaatacactttc, accaggaccccctaag,
tgttatactagctact, gcggctccagaccgtg, ggttaaaccccgttct, tggatgaaactcttag,
tgggtgacttgactta, aggagtacttttggtt, atagggagaagagacg, gcatcatacgcatcta,
agtataatctaatcaa, agaagtcttatgtagt, ctacctccccccccca, agtccgcaagctgtct,
gtccacatgaggactg, gctgaatacacaataa, cctccaatcccctgat, gatactttgctttagc,
cactgcagtacgttaa, gtgcgctgccagggca, acttttgtcttatggc, ttccctggaggcagcg,
atgtgggatgacggcc, tcacacccgcatccat, aagagagtctcgaaga, acagctataccctgct,
tacattttggcgagtt, attgaatactgctaat, aagtcttacaggttgc, tagtggaagtgtagta,
catgagccgaaatccc, cttagtctcttgttag, accggctcctcccatg, tatcggtaaaaaatat,
cacaagtatactggca, ctcagggttccatgac, aatgggtttccagtta, taagttgaagtatatg,
acagtggctcatgtag, actacaagcgcactcc, gggcttggggcgcaac, ggaacacatagggagg,
gaccgtgcggccgccc, ccattaacacagtgtc, gtgtcttagcatttag, gcccggaggcccttcc,
ataatcaggcccactc, ctcgtcccggtccagg, tgcacgggctcttttc, taagtgcaacctgtgc,
gctaacaacgagatga, gtgcggccgcccacag, cgtttatcgattttct, cattcttaaacacgaa,
ctggtctgtgtcacgc, gccagtctgcaaagtc, ccctagcgtctgtgga, cacttgattcacccac,
ctgtgagattatggat, cagtaggctatgaatt, gcattaagcatctccc, ttagcaagtttgtagt,
aactgtcactccggtg, tctgcttaacgcatgc, aatagacacaacaatg, cttggatccagggtca,
cggaagccgcatcccg, aaacgttatccttgtt, cacttccccgagcctt, caccgtgctcgagctg,
gggtactttaatgccc, gagagcctctcctaag, cactctgtatatgctc, accgctaaaaaaaacg,
tattatagcctcgctg, ggttaagcacacacag, aagagttaaactacgt, gagtccagggaccgtt,
cttagtcataggttcc, atctatcacaggtgag, gaacaggtgcatctct, cgctaaaaaaaacgtc,
ggttgtcaactatgag, ttgttttgggcccgct, taatgttctcgttcta, ctgcatcacgtgatcc,
atgccaggatcccttg, tgatggccttagagat, gattgcccccccccga, agacggcagctaacgt,
agtacagctgtctctg, atcctactacacccct, attggcttggagaggt, tgacttgattgtcagc,
ttgataaagtcacccc, gatctgtctggattgt, ggcacttgaattatat, actccatatccaaatg,
tgtatgcttaagataa, taaacattcctggtca, cctattgggagcggca, gtaactgtataaaagc,
ttaaaatcccccccca, gaatcgtgctgtaatg, atgttccagccctcga, gccaattctatccctc,
gtgccccctattctct, ttgtgtggttgagtgg, ttgggaacctaagatt, cagttccaccccgggg,
ctgcacccgcgtgccc, gtgcttgccggtcgtg, ccctaaatggagaact, tggtgccttacttaca,
gattagtggtttactg, ttcgggtttgttcctc, cggtcattttttttac, ctacatatagctctca,
ctgacgtccgtaatcc, tcaacattcaggcagg, ttaccctgaacctcat, ccagggggctgagctt,
caggtaagcaaaccag, taattttatccgaata, tcttataggacattga, gcgcacaaaaaatttg,
ccaatctactcgggaa, taaaggcagttatcga, cttccagccacatatt, tgaggcactgtgcttc,
gacatgtttaatctta, cgaaaaaaaagtgttt, agccggatgtgggtgg, gctgtccttatgtctg,
acagtcaggggggaaa, tcggggaaaaaataga, gtaacaagttcttaac, ttagcctccaacacgt,
cctgattttcctgccg, cgctataactccgctt, atatctatctgcaggt, tcaaatacaagcggat,
ttataacttaccagct, gcgacaaaaaaaggca, ggagattaagtgcatg, cacagtcaggggggaa,
gtggcggatatttgca, caactgaacgaagctg, ggtgggcagaacaacc, aggatcaccgtaccct,
gattagataattgagc, aacgaaaaaaaagggc, ctctaaaaaaagtgcc, ggccaggcttatggca,
gatgtagtgggaaagt, ccggttaggtcaaggg, gcttcaagcaatttgg, actgttacttcttccc,
agacccttatgcacac, cctcacctagtgatgt, ctatactgctggaacg, tatgaggttactcatt,
caagggattttctggc, actcatgtcgctgact, ttggctctggctgtct, taatctgaggggataa,
tctatgaccccttttg, gcgggggggtacttat, cttaaacctaggggga, gtctcgaaacacaaaa,
gttttgcaggcttagt, ggaggtctgactatgt, tgcatgtttcaattag, tagggtggagggttag,
aaagtaaatattcgag, tcctatagtcacaatt, aataacaaaaaaaacg, ccctcaaaacagaata,
ccggggggtagctttg, attacagattgcttaa, ggtgtctggcaactag, tttgtaggattaaact,
tttgattgcagtttct, gcctttctgggtagcc, acaggttgattactcc, tgagaccttgtctggc,
gtactcagcactgggc, tagcatgtaaaagtcc, tccaagcgtggttttt, ttgagagatggtatac,
gatgtcgacagcctta, tttaaccacgttgtcc, gaggcgaggctgcgcc, attgggctctggtggg,
agcaatacacggccct, tagactatttgtatca, ttccctgtagtgtagt, caagagctatcatctt,
aggccctagagatgct, ttttactcggttgccc, accaaaaaaaacgggg, agagaataaactgctc,
tgaattactccacctc, aacagctcggttaata, gacgcagaggaccatt, aatcaatgactgtttg,
tgttcaagagatgtga, ttcccgctttggttaa, ttattggggcccccca, taacgcattgttcgga,
tcgtctgaggcttcac, tgaaacactatactcc, gttcttccagttaggt, attaatcctgattcta,
actggttttttttagg, agaccaaggtcctttc, cttccgcactcttctt, ccccgccagcgcatta,
atccgatcctctgtgg, ttttcacagcatagat, gagtaggagcctccgc, cacttaggacttccta,
tatatcataaacatcc, gttactgccccccccc, cttgcgtccaagtttg, cataattgaaacacac,
cactaaagtagctcac, caccagggtttttttc, tacccattatcactca, ttgtgtttggaattac,
attacctagtcatggg, tacgtggtgcgtggcc, tccatctcgggcaaaa, acaattatggtggacg,
cttgcaggaagagcga, actcccaagctattaa, cacattcaagatgtcc, ataaaaatggttaccg,
cgattttttttccaag, tctctaactaacaaaa, ttcatatcctgtctaa, gctctataaaactact,
ccttccgcaaacatat, tacttgtgccaggttg, aggcatctaggtgggt, gttatgatatgcaata,
ccaaatgccggagtag, gccccccccccatgaa, ggttatgatgctttgg, tgccccccccgggggc,
gggaggcttaggaggc, ttaagtagggtaactt, ttctggtgtgtccctc, ctttccaacccacatc,
accccgtgacagagca, gtttcggggggaggtt, ccaccccgaaaaaaag, tctgcgatgcatcttc,
catatagcagattgtg, caagactaataggtaa, gcgttccaccatgctc, tcagttactgaacctc,
agcttggcccggagag, cgcgggtccagcgcct, actgttatacttataa, ctatactctctgagtg,
ttttttttcgaaatta, cgctgcttccagccta, acagactgaatgatct, ctgtggacttaaaagg,
agcctgacgcttctgt, tgaagatgctcgtgat, acatggacagggagtg, ctgaggtagggtagga,
gtctaggcaagacttg, catggttaaaccgtgt, ttctccttgtctacta, tggttacaccctctta,
ccatgcgatgctatag, attattgagtctcgag, tgacctgatagcatcc, gattttttttagcgta,
agaatgtccagaaccc, tgagtaagaacaagta, tactcagcttaggcag, cacattaaccccacca,
gattaccaccctggtt, aaccatgcaacaatgt, agtccatcggggatcc, atccggtagaggtgag,
cggagcttactgagag, gcaaaacggtttatga, ccctggcagataatct, aacggtaaaattaaaa,
ttagttgaataatcaa, caactactctaggtgc, ccattgttggagggac, ggggaaatgtgcgcgg,
atctcaagcaattaga, gcctcggtctcctgca, cacgaggacagtagat, cacagtactagagatt,
acaattagcgcctggg, tctgacgtctgtaatc, ttagctgtttaagtct, attacagttaacacat,
acacgactcagaatga, agcatgattgtgatcc, gaacagtcgttgctta, cctatagtataaacac,
acagctgatacttgca, aactcaggccaacatt, atggggggggaagcct, atttttagtagcgtga,
cagaacaggggcgtgt, gagtagcttaaaggcg, gcccgaacacttggtg, acgtctgcacaagact,
tgaggtgttagctgta, gcatgagcgacggaga, ctgttgcagggttatg, ttcttctcccagcggc,
atgatttcagacacct, tgccacgaagtccaaa, accctccgggagggtg, ggcgggcctgacttat,
cttcctttgtgcgccg, tactgctgaaactata, acatataggccatcaa, tgcatcccaccctaga,
ggaggtgtagatagct, aaaagcgtagcgaggc, gatgacctcgtggtcc, gaatgcacgccgtagt,
gtagttttttgtatag, gatttttttcttgtcg, tgtaattttttcggtg, agacctccccccgaaa,
ggctaacatggttaag, aaaagagactttatcc, agattaccctttttct, ccactttttttatcat,
actgccgagttattag, ttccctttgctccgtt, tcatagggcttcatgg, acaaaacaattaccgc,
gattcagaggaaaggc, actcctggctgggcta, tgcttaacactaagcc, atgattaaatcgccaa,
tcgttgcttatgataa, tgttgggacagctaaa, ttaggtaaagatcaac, tcggtatttaataagt,
cactgcccttgtgtgc, tatgggcgcccggcta, tcccaggtgattagcg, cctcaagtttctgggg,
attccacttgtacgat, taccactgaatagata, ctaccaactaaactcc, tgcttattgcgtaagg,
ttatctaagagagttt, tgtgaggcgagtatta, ttttacacatagggat, gtcctaacaagttcgt,
tattccgttctttgaa, ttcaaaagggacttat, ttgaggaatttacaca, gcttagttgaatggta,
gccctataagggtgaa, caaccccccccaaagg, actatcccccccaaaa, gctacccggaaggctt,
tgctattggagctcga, tggttagaagttatct, ggtcctcgtgggcctc, gtgttagccaatgcac,
gaaaatatgcatacgg, gttttataggggtaat, ccactgacggtcacac, aggcagttacaacgta,
ttgtcccccccgaaaa, agtgacgatacgcgag, actactctaggtgcac, ctgctttgccccacgg,
cagtgggtgcttgcac, aagagcccccccctcc, cagggaatcaactggt, cactcataatgcaacc,
aagctcaaccaggtgc, ctgactcctttcgctt, ttattacgctgagaaa, atacactttgtctcag,
atgagacaccteggct, cteggaggcacaagaa, gcttttttccccgctc, gegccccccccactca,
tgcaattactatcaag, aaccgaagaaaggagc, cagatttcgcatcaga, ttccagcaaatggggg,
gcaaaaaagggaggcc, taggaggtcttcccgg, tctaggtgtctctgca, gcgatggtgaagtgga,
tatgcaacaaatcttc, tctatcaccttagact, atttgatatacgtagt, tattggatcttggggt,
gtgcattggtgtagta, caatcaggggcacagt, tctaaaggagtggttg, gttagttggaaggaat,
ttgtgagccctcaaac, catttgcacaacttgc, tacagaagtaaaggct, cttcggctctcacacg,
ctagactgctgatgat, tagtgtatggctggca, tgaattcaatacgttt, ttttgtggtggcgaat,
gcggtggctgagatct, atcgtggcataagggg, gttcgcccaagacagc, tcctagcatatggtag,
atccacccagagttgt, ggcaatactcatcatg, tggtcccccccgaaaa, accaggggcaaacctg,
tcttaacgtcacgctt, ggccttttcacgctga, ggatcaccgtaccctt, ctgtgtattacaatga,
cactccctcaagtctt, gggcgcactattgctg, aacaatgatcctttgt, tctggatgcactccaa,
catttttaagcccgtc, ggcttatgcccccacc, tgtctacaccgcagta, ggtgacgcgcctgcca,
ttccattgcccggatc, accactgacggtcaca, gatcaatggtgagagg, gtctcttcgtcgggac,
tactgaagagggtgag, aggacgcagatgaggt, atggattacttaaaga, tgtgcattagttatat,
tccgtctagggacaaa, accatggaggtcttag, cccatactttcatatg, ggtttttttttgaccc,
aggccttacattcctg, gtacatggggagaact, cctagttcacagatac, ctgcaaaacttatcgg,
aagcctagtactgttt, ctccctctcgtgcgga, gttgaatgttggaata, tcccgggggggcccga,
tctgtgtctcatacct, acatagtgctctccag, gtgcagggataatcca, tgagaaaggtcagttt,
agctgagcgccatcgc, tcccagcccccccctc, ctgcaacttcaatggg, ggctcaccagtgcact,
cccggactaaaaaaaa, acacagcgcagggagg, catgggccaagttctg, tactagtgatctgggt,
cccccccatatgctgt, ccgtcaacagtgccag, gtattactcaaacata, gcatgcgtgctcgcgc,
gtgattaagagtcttc, ctgccccccccgaaaa, tgtgagctttacccat, gctccgacgccggcta,
tactgttcctcgaaaa, gctgccgctagcgcgg, attgctgagaaaagcc, cgtagaaatttggtgt,
ggcatactgactcttg, cattctattgataatc, agcatatggaagccag, atggacttgctgacgg,
gccctttggcttgtgt, gtcgtcttctcctttc, agcactataattacca, ttcttcgcaatagaga,
gggaatgccccctgcc, gcattcggatgtgaat, gggtcccgcgaggcct, ccctgaagtgtgcgtc,
gggctaagccctcgct, aatcaccatgcctttc, tcctacccccccccag, agtaaagtcaatctac,
cctaagcccacctttt, gcaaactattgttact, cgggccacaggcccga, taatcatctgtgtaag,
tgggagcccatgtagg, atgatatgtctgcctc, gtttaggggttcttac, cgtaaaaaaaatagta,
gtgttattgttctggc, ctgacgccactccaat, acccgactctactcaa, gtcttcatggatgtct,
gaggaacccagcatgt, ctaaattgaggtgggg, tcctgtgagccgaggt, taagatccatctaggc,
acggaaaactccgcct, cgctagcgcgggtggc, atccctgctaaaatgg, ttgtgtatggaaactt,
gtttattctgtaaggc, tgagatagcttagaat, gtgcttcactcgcccc, ccagcagcctccccgt,
acaataccaagcgagg, atcccatctgtataga, tgatgcgttcttccca, aacgaggcttaaaagc,
ggtgtaatatcccgaa, actgagaggcaatatc, tttcccaacctgattg, atctccatctgccggc,
cctccgcaggagagca, gagaaatggcccatta, cttgttagttaatcgc, cctattagctgtttcc,
aacaatgtggagtgat, atatagccccccctgt, gaactaagttgtggtt, aaggttatccacaact,
tcgtcaaatatcccct, accgggcgctaggcgg, ctgtccaattgactgt, gttggggggggaatgc,
ttacccatagagagca, gctcttaacaggaaag, gatccaggctctcaat, agcatctgaggaccta,
agccatctgcccttaa, tgaattgaagattctc, ggcttagtttcatctg, gtggcacccacttgac,
ccagtatctcggcact, caatattcgtttgtac, aaagagagcctgatac, cgaggcgctcgagtac,
tgggttcagcaccagc, tgttttcctatgggac, tcaatgccaccgccct, tataggtgcccttccc,
attcttgagcaggcca, ttgeggacttaacccc, caagaagctatacttt, tgcctcctccgtttct,
catcttgcgcgagccg, agcatacgaagttcag, atcatccaatcagaat, agttacgaaaaaaacc,
cggtccttccgggggc, gctcgagtacgagcga, tctgggtccccggttc, tccagttgcgttattt,
cacgtctgtaataaaa, gttgcctgtgggtaag, atcccaatcaaaaaac, tcttgcagttcagacc,
tagttgggcaaataat, acgagcagaattgatg, agtgacaatgatgtca, ttgtctctagatgaaa,
ccgggccggccttgct, aggtgctaagtcctca, accgctccagtctgca, cagatggccccccccg,
acgagtatgggggggt, ctcggtggtttacgcc, cgcaggagtagccagg, atgctacctactttca,
aaccccccccaaaggc, ttgcttgcaatgtcca, gagaactggacatccg, agctcatagctgataa,
tttcgtgagctaattt, ggattagttatacttt, aaattgttcatagcga, aagtccattacccctg,
tatccacgtttacaat, accgacatggtgaacc, cccctgaccaagttag, aaacgttctgaatgac,
tagcggaggctgggtt, tacaggatctcgaaaa, ggttagtgctgttcca, ctaaatacagcactgc,
tggggattcctgctaa, cctcttaagccacggc, ggccaggaacagggtc, tttacgccaagtgccc,
ccggacttcgtgatct, ggattgattttccacg, acaatctgttggaggc, tgaatacggacttatt,
ggaatgtgggatgacg, atgggggggatgtgga, ggacccgccgctccct, tattaggtagcgcaat,
cgaaaaaaaatacgtt, tattacccggataata, taatatgggggggatg, aatcaaaaaaaaccat,
agcagctggtaggtgg, actcatctcctcgagt, tgctgggtggatgccg, ctaatatggattccca,
aacagggaggttttat, gtacaaaaaaaacggt, aggccaattgacatac, gctcactttagtgtag,
gcataaagtagtgggt, tttttctgacaacagc, tcctcttcccccccca, gggacaccgcagggaa,
tttagtgtagacttcc, taaccttgtacctcaa, ggtatctatcccgtag, tgactatggccccact,
ctcagtcgtccaagta, ccagtattgggggggt, gtgattaggaagtgtc, cgagacagcgggaggg,
ccacatgacgggaaga, cctcatagactactaa, aacttccgaattaata, cattgatacaatcatg,
cctgggccagcatagt, gactaataatcccagc, accaagatttcggagt, cgggtcttgatgaaga,
gttgtcccccctgctg, gttttaaaggggagta, caggcggcgggccggc, aatcaagtaagattgt,
ttggatcagaggcacc, agcatttcccccccag, ttttatacacccccca, catgttcatagcattg,
ttgtataattgaacta, gcctgccccccccggg, cttgcctgtctggaca, agtgccactgtatcgg,
ctagaatagagctcgc, aggctttggatctgta, ccacaatagaattcat, cgtgtttaaatgttga,
ggacgctaccggcctt, gaggctgcgaggcttc, gtgagactcattaaac, gagcaaaaagtgctct,
ggccagtttaccctcc, acgtgatccccaagtg, cgcagggcttgcaagc, tgcatctgaatctgga,
atacattgctcatctc, acggcactaattgttt, tgagcttacgaagata, cctgaacattcacgtt,
gttagtgaccatggtc, ctagctccctgtatgc, aaatacgtgaccgttt, ctaggctatggctccc,
cttagcttattctagg, ctctataatgagcaga, tgcatgttagcgaatt, aacgcagccaggaagc,
tgaaaaaacaacgtca, tctcgtagctcaggca, ctcattttttttgacc, cctccatgtatcacga,
cccctcgcaccctatc, gaggacagagccgcgg, tgtgccaactttggcc, tgccttaaaaatcgtg,
cagaaattgaggcctt, gcatagccttccaaaa, caatcagctcgtggac, aggggcgtgatctgag,
gctggattcataaagt, cagtaggagaacttcg, ttagcaccaatgcgaa, ccttttccacgaaatt,
gagctgacactattta, ttaacgaatgcattat, gttaggtagactctgt, gcacataggagcctgc,
ttacgacttcctgggc, cttttgggccccggag, aagaagccgcaaagag, tctatgccccaactaa,
ggggcttcagtacttt, atcattcatgcttatt, cacagtctggttgtaa, gtactcctgtctctta,
atccttttaacgttat, acactttttttatcat, tctatttgcttagagg, ccaataaccgagaaga,
tgcgtcttcgtagatc, tcacgtttatcgtatg, gaaccttgggggttgc, gggggttttaccaggc,
ccaatacttaaccaaa, ggttgcttcaaatacg, agcccagacgacagcc, agttagaacacgcggt,
tataacgatcattaca, cagtggctcagctacc, ccattgggagcggcag, accagcaaacccagtt,
tcgaaaaaaaatacgt, acccataagggtgctg, ttggcaaaaaaacggt, gtaagcaccttagagg,
accgatgcttaggagc, acatgttgtaaggcca, gcgtggcgcagggctt, ttcccagggcacgtgg,
catgacaaatacaccg, ctgttactctagccat, ggtcaaaaaaaattag, atctgcagtgtgcagg,
ggcagcttgtgaccca, acaggacccctgtggt, gccaatcaggaaacca, ttatacttcagatctc,
atcaccaactctggta, ctgaatcctccctaac, cagtagatggaaatgg, tagtgctctttagtta,
tttagtccagcggtct, tgtgagattccccctc, ccacacgggcgatgct, taacttagctttaata,
atctagagtaattacc, atccattcagtaacta, tttacaattatggatg, tctcgaatggtaactc,
gagcaacctccttcgc, tgatattctgataggc, ccgcatttgccaaaca, tagtttttttttcgta,
ctgcgttgcactccag, tcacccacgttgacct, ttcagatagagttaac, acggaggcacgggcga,
ttcccattctgagtta, aatgagcacttggata, aattttttcacgagct, gtttcggctgaagtca,
tgctcgcccgcccccg, gtcttaggatataatg, agcgtctttgctaggc, tgctctccagaaagcg,
caggcctaagtaatct, ctccacccaattcgat, ctaacttctgcacata, ctgccatgctacctcc,
ctgtagggggttcttc, gatggctagagtgtct, caccatactatgtcga, atgagccaaactcttg,
gattcattgtatacac, ttggaagtataggctt, ctatcttataaaggtc, ttattctgctacctca,
tggccccttcaatggg, tgctgcaggattcctc, tgggtgaagtcccact, tggagcaacactacta,
tacacctcccacatgg, cccgggcgcactattg, ggggggggacacagtg, acctgaattgtgccat,
gcttgtcctcaactag, gctcctgctactctgt, cctcccgcccttgcgc, aacctgtctccttcga,
cccaataacccacaac, tggcggatatttgcat, cgatgcttaggagcac, atgtagatagactttt,
cccgatacgagtctcc, agcctgaaaggtttat, ctgatcagacacaagc, agcaggttatgtcacg,
catgggggaatgcccc, tgaggggtggatagaa, aatttgttaggggggg, tggtggccattggcaa,
ttctcgtttggccagt, ccaaacagtgcatcat, tttctagttgtgagtt, agcccggaatccccaa,
cacggttagaccccgt, cttagaatttaggggt, ggaaaaaaatatcgaa, acggggggggttacaa,
tctgaggggataagac, cggggctgtcccgcgg, ccccccccccaaggga, ggccttagacggtgac,
ttagcccccagatgaa, gggtgaagacggatgc, tgggcttatggtctcg, agacggatgcgggttc,
ctgcggactgcagtgt, cggagcgaggcgctcg, actgctgtgcagtctc, tgctttcactctcgtc,
cacccagcgagtccat, ccttgcgagggcagcc, gactgctatggattaa, tgcaaaaaaatggggt,
ttttattcggtgctat, agatagtcaagacgca, ctccgggtcttgatga, tgctaagagctgttgc,
cattctgtgtcatagg, ttgatcagcgtcaatg, taaaatgcctcggctg, ctcccagtatggtgcc,
atactaaaaaaaacgt, gccgccagcgctcgcg, gaaaaaattgcgaaaa, actaattctggcacgt,
gcgggtcctgattgtg, acatgcaattgttcct, atgggcccgcaggaat, ggtactcctgtaagat,
agatgaggtacgggcg, ttatttcgggttttaa, ctctgcaatatgtggc, ctgcctatgcacacaa,
ttgcagcggttgtaaa, gtgcttaagacaccca, gaagaggggtagtttg, ctgtatatagaggaca,
aactccctgtggtggt, tgcaccacttggagac, ctcaccgatgcttagg, tgcgcatgttttattc,
taccttgcattttgga, gttaaatggtgagagt, tctgaacctgtaagct, gtcttgcgggtgagaa,
tcgactttttacaaag, aaacagattacacgag, tcctgcctgagctgta, agccagttaaactaga,
gtcgggattacatgcg, ccccatggacctggta, caaaccctactgccta, tgcaattgagatattg,
agagcacatcgattct, tcatcctacaactgat, tggacacccaccgatg, gccattaaattcatta,
gatgcagcggatcctt, gcctaggggcctcgca, ggacgcagatgaggta, aatccgtggaatgttt,
acgactctgctcctcc, tgatgtccaccttatg, tcccctagtaacaaat, agagaaccccgaaaca,
actagtaggcaaccgt, gtctgactgcggactg, tttctaacatgccccc, cccagtatatcaggtc,
cagtgagggcccccta, taaaatgtcggaaata, cttggctgtaagcaga, cagtcaacaaatgtcc,
cggagggagtaggtgg, aggatataggatcttt, tgcggtctgggtttgt, tctttggctgatatcc,
gaaacttaatgcacaa, ataaaaggcccatcat, cgcagaaattcagagg, cactcggattttttta,
tcagttccacaataag, tcattgcatgtaggtt, acagacgctcatgaaa, aatcgtgtagaacgta,
gaacgcacaacttata, gagcatgtaagatttc, tccttaggcttaccca, ctatagacgtatagac,
aggcttaagcgggaag, atacagcctctatttt, tcgacacggcttctag, ggcactaagggatact,
gactccattccccata, gtttgggcagaatgac, caaaagcataagtcac, tataatctcaaagtcg,
accttccggatgggca, aagggccatggacaag, gggaaaatcgattttt, gcttattaacataagc,
gattataaaaaaaaca, aggcggccttgccgcg, cccttatgacttttga, tactccttcgtctaac,
gtggaacttttatggg, tttgtgggtagttcaa, aacttatagctaggca, atagaacaagctccta,
caagctacgccttctg, ctatatgcgtaccatt, cagctaggcggggctt, tcgagggtcctcggct,
agagtaaggctgtatc, ataagctcggtcacac, gcaccgaaatactgtg, tactegggcagcttag,
tggccccccccccagg, gcgtatcccatgccca, cttcaaaacatctacc, cggtcacacataagct,
gcaccagtatttttgt, taatgctcatgtccta, ggttgcaaagtattgc, cgctggccagagccta,
atcttgaaggacatag, ttgacttccactattc, cgggcgctaggcggct, gcccctgagtacgaat,
gcacattctgcatagg, cttagctgcagccccc, ggttactgagagtaac, agtgggtcagggtgct,
tggatcagccttaact, tataaaaaaaacgttc, aggcatctccccttac, atgggggggtgtctat,
tacgtctcttttaata, cttcgccactttttta, aaagagcacggggttg, atgcacatgtggaggt,
gacagcccaagactgt, ttgtgggtgcagtatg, aatggcccccccccca, cgatgtcctgaactcg,
gggatcgtctgggtgg, aggctgcacagttcta, cagtccacggcttaaa, ccagcttacactttgc,
cattcattagcaaagt, ccaaccttttgggggg, aaccgaatttttttga, ggggggacacagtgcc,
gtgttgggacagctaa, ccagacgctccgcctt, tccccgtgggtatggt, ttgagggtcgcatccg,
atattgctggtatact, ctacacataaccaaac, cgcaagctggctcacc, gcataggctagaacaa,
gaccccgccctgcacc, ttcattttatccgttg, gccactacaggatcta, acctacgaggtattta,
gagtatcgtggggggt, tcatgcatggcgggct, gcagcgcggggccctt, cctatcttataaaggt,
acactgggaggcgtag, gatatgaagattaggg, tgactttgtggtgtca, attcatatctggatac,
tgaccataaaaaatct, aatggggggggtgtat, gttcttaacgaaaaaa, agtgatagaagggctg,
actccatggtggtgtc, tctgcctaatatggat, gacacattgcttacta, attcttgcgtcttcgt,
cgcccttccacccctc, tagggcaccaccatac, tccttttgactcgaaa, atactggaagatccaa,
catcacgaacagtata, gtatgacactaaggtt, cagcacaatgtgcgct, tgcaggtaaataagat,
actcttaaccatgtat, tcctatgagatataca, cacgatggtcggctaa, atccgaaggctcacct,
gattttttttccgggt, cacggtagtccagtag, ccgtctgccccacatc, gctagagagctaaaca,
aacagacacattaaag, accatagctgatgatc, ggaggggaggctacgg, accaccaaaggaactc,
aggcacttacacatag, ttagaggaagcgagag, ctgggtgtccaactgc, ggttagcctttgcact,
tggtcaacgcaatttt, taaacacatcttatct, ccgaatgaacccctga, aagggccatgtactta,
agaaaggggggggcat, cttgccgcagtcagga, gtcccagtataataag, caaaaaagagtgcgca,
gactccccgcattgcg, tgtgtcaaatttcacg, cccggaatccccaagg, attgatgtaggcttga,
gccaaggtgaagtgat, taggggggtagacata, agtgcgactgtgtcat, ccctttgttaggctga,
gcggcacgatgtcggc, ccccccccagaaaaga, gatgcatcttcttgcg, atgtgagggggggtgt,
tcagggaaatcctatt, tctccagtgtctccgc, cagttgtcccccctgc, tggaacttgtttggtg,
atcccctattaatcgt, gtagcgggactacatg, gcaaagcgcagtattc, ctgctaacgtttaaag,
ttccggaatggtgagg, ataaaatgcctctatg, ggagaatgtgttccgg, taggataagaactaat,
atcctatctcattagt, ggagattctccatcct, tgtattatttaggatc, gattccctgaggtaag,
cagtcttggcagctga, gggtcctgcagctctt, ctgagactgctatggt, aacgcttattgatggt,
agacgcggcggggttg, accactaccaatgaca, ccttccattgcccgga, aaggcctattatgatc,
agtctttgttacctag, tctaagagtacccaat, atctgaaaaaaaacgg, aggtacgaaaaaacat,
taataatcactggccg, ttgtctcctatgattc, ataccaagcgaggcca, ttagtaggcacagcgt,
gcactccaagacaaga, cctgctcaaggtggag, gttagtatcaaagtat, tatcttcatccaaaca,
tgcacgcagaataatt, catgcccggcggcatt, actgtgtttggtggac, agtcccccccgcccgc,
tgatgaggtacaaagg, gaggggggggaaaaat, gacagtccacggctta, atatctaacgtatata,
ccggtaggtagccagt, gaagcaaaacttaggt, gccttgcaatgtccag, gggaaaaaaagggatc,
gcaggctggggtgtta, ccatccttttttggag, cctttaaatctgagat, aaattggccggtcgct,
gtagaactgggttcaa, gagtcccccccgcccg, catgttttatcgtaat, caaatacgtgaccgtt,
gactccacctgcaaat, tttcggagttgtttgt, tggggacgtcgaaggc, tattgacttcgtcaaa,
gtgtggtaatggtaac, ttagtccggttcactt, tcttggggggggttgt, cgttccacctcatcgt,
tatagggatgtgtggg, tctactgaggggtttt, accgtctttttctggt, ccatccggagcagcgt,
tgtatgcgcatcccta, gagcggtgagtcatct, ggagactgggctcaat, attagcctgcgtggtt,
ctctcgccccctggac, gcgagcaactgtagag, ttcacaaagggccgcg, acagcccaagactgtg,
tagacctgcaggaaca, gttgatcatcactggc, tgaagtcttgacattg, ccgtgggcggcttcct,
tgtgtcccccccatca, gtgatgtcacatctcc, atcactcccccctacc, gaggctcgcacccatt,
tacactcatataacac, ctacgattaaattacc, ccgggagggataggtt, tcgtggacacattatt,
cccatcccccccccct, actccaattttttact, ttggggcactgaaacc, tttaaatgccccggta,
gaagtcagggtcataa, tctagagagggtggag, actctcggggttgttg, tctcacccgctctaga,
atccagagagtagaac, tgagtgatatcatgtt, gaaccacaagacgggc, cccggatcaggaatgt,
tgccatcacatggtag, aacaggcatggccaat, cagcatatcttggcta, agacaccgtgagcatt,
gctgcggtggctacgc, tgcatcactggtgtgt, tttaccatgaacacac, caatcagtttaactat,
aattagggggatcact, gctccgccttactatt, tctttaattgaggttg, tcccgctgcccttagg,
gcttattgcgtaagga, aatgcgaaacttgttc, tttaaaaggcgcatgg, aactagagcaacttaa,
ctacgcccacggattc, gatcacgacatcagcg, tgaagtgtgggggggt, tagggagcatgctcat,
aagaggggtagtttga, gaacttcatgccagga, cagcccaagctgttag, gcttggggcgcaacat,
taaaaaaaggcggagc, acagctcggttaatac, aaatacctctggggca, aagcaaggaagtacac,
ctaaaatggcttaagc, gtccgatctactcggg, acagacgtctggaact, gtcctcctctttgatg,
tgaggaattgaaggtg, acttagctggctctga, attagctgaggtacct, ggctgagcgtgcagtg,
gttagggggattagga, acatgagagtgtcttg, ccaagagtgtttgtgc, gcttccccagcggccg,
agttggccacctgtta, caacctgctactcaca, gttgttgatctggtca, ctgtagttggctagtt,
cgcatctgatgaactg, agaaaacgggcttcca, tgggagctaagcggtg, aatacaggatatgtac,
cgtgggggaaacccct, aatcttacattgacta, gaaaagtaccccccct, taactttcccccccac,
aataatgttgagctta, agtttaagatgactac, tttttgtgccggcttt, agtgctatcatggtgt,
cattacgtttgtctgc, gaaaggggggcatagt, ctcagcggctctccac, ctcaagcatttgagtg,
aacgcacacattcata, tacgcgttcattaggc, atattgacgattttat, ccccccagtcccagcg,
gttacacttttgtatc, gttgccctacccccca, gtgacaggaataatgg, cactccaggttgttag,
ataatcggaaactaat, ttgatgtacttcccta, tggcgtcactgttctg, gggcttatggtctcgc,
aaaatagtttaaagcg, ttctgactgctagacc, acttactccaaccata, actggtttttttagaa,
caatttagccaggatg, gtttaaccacgttgtc, ccgcctcaaatgaaaa, aaactcgtaagcactc,
atgcgggtggatcaag, ccttgctttacgggct, tggcgcttggaaccct, ttgttggggctttggt,
tcccgggagagtccgg, ggcaccatttgacaga, gctctacttagtgcct, cctccgcattgttata,
aaagcaatcaccgtct, agaactgtgtcacaat, caggcatccccgattc, gggctctctacaaaga,
gatatgtgggtttctg, gccttttttgcactac, cgcgcccaggcctgga, ttcctattagccccag,
attctcaagctcaact, gccctttttttaacat, atcaagtgttctatat, agaatcaccaggtaac,
tagaagagagaccaat, ggggctgtgccagcgt, gatgccgcatgggctt, tccattgcaagccttc,
cacgttactagtctgt, ttagcccttagcaaat, cacaatccccctcaat, tgtccacaatcagggg,
tgttcaatcattaacc, agtttagagggttctg, gagattgtgacgttaa, tagacaaagacctaat,
tcgtccatggaactag, aagccatttagcccgg, ctcgcgcacggtgctc, acactccatctcgggc,
ggctttttttagcttc, ttagaggcaacttaat, caagtgcactgtcctc, gtagaaatttgtcaag,
gctcaaatggtaatat, gagggcattttataga, tccctttgcagtacaa, aacagattagatctga,
ttgtggatatggtaag, cagttagaagagttca, catgtgtataaacact, tttaggccaaactcat,
gaaagaggcgtatttt, cccccgagactgagta, caggtttcctgagcaa, ggggaacattaaagat,
cccctcgacgcctttt, tatacattgctagggt, gaccctcgccttgagt, aggaatacagactctc,
ctgaggctatagcttc, ttctttagacacattg, gctgcggcctgcgcac, tcttagcactgatcaa,
cctcagcaaggttaag, tataaagatagctgct, taactaagtgtacctg, atgaggaataagtcat,
gggtggcttaggcggg, cgatgctgagatgctc, tgattccatctcagcg, gcagagaacttaccac,
ctgagcttacctctca, gatcttgcgaccaacc, tacttaagggtttatg, ctcacaaccttcaatg,
atgaggagtgctcact, ttacctatataaccca, cagtgtaaggtaagtc, ccccaatgtaaccatc,
cctatttcgattgaag, ctcatggacggggcgg, cattcatactgtgttc, ttctggtctatagtgc,
tcccctattaatcgtc, tgcgcgctcgcgcgtg, gaatatgcatgttagc, ttacgtacatcctcag,
ttgtgcagcccgtccg, atggaagccttagtca, tgcaccaaccactttt, ccagttatcccctcat,
tgaccagcatggttaa, atgaatcctaggatag, gccctgctctgtacct, gagggattgtgcaatg,
ttcggtggaagaggag, ggctaagcggagcggg, caaaaggtacaggtgt, agtgcctggattaatg,
gccggtgttccctttg, aatggagtggacctgg, tgacctggtgatgtac, gactccggagaggggc,
caaaagtcgaagttca, cagactgccttagagt, ctgacttacgcattcc, ccctttctgttaactt,
tttctcttacgttgct, ttaatctatgccccag, tataaatcttaagccg, agtgcatgccccttat,
ggacctaccccctccc, ggaagccctgaggcat, acatgtcgctggatct, tcttacccaactageg,
accttcctctccgcca, cttactgcaaaacggt, ttaaacacattggatg, cctgcgcaggactatc,
gaacttttatgggcac, aagatgcttaggaggg, gccatcccgagcatgg, acattttttctggtcc,
tcacctcggaaacgcg, tagaggaatactacta, ggcacttttgccctgc, tcccggatgtatacag,
cacgtatcagtgataa, acggttagaccccgtc, tgcaaatgagtgcctt, gtctttggctgatatc,
tatcccataaaaaagg, cgttatgaagtagagc, taggcatgcaaaggct, cctcccttccttcggg,
cccccatgaatttcct, gcttaaactataggca, gacatcagcggttcaa, cttagtagctacaaag,
aatttatcaacagagc, aactctatctgttagt, gactcctgaggcacta, agctatgtgtcaagtc,
atttctatcgaaattg, aagccccgcacacaca, gaaaactctccgaagt, atcaaaaaaaggcact,
gcttctttgtcgagga, tccgcactcttcttaa, caaaaccgtctctctc, tttcgtcttatcgaat,
gtatgtaatgttgtct, ctatggaagggacata, gttgggccgggggggc, tccaccaaactggggt,
gatctggtttgcacta, aaaaatataacgcagt, gcttcccataaataat, tegtaaaaaaaagggt,
aatgaatatcaacctc, gcacctgtgtcagggt, aggctcaggcctgtac, tcaagacacccaacga,
tctcgtgaaccctgga, atacaccgggcctgca, gtaatgtccacttgta, gcgacaggagttaaac,
gtaccttagatctgat, attaacagggcaatgg, aacaactgacaactaa, tcccaatcaaaaaact,
atctatgtcttattgg, tatgagaacctcattc, gtaaaccagtagtgtg, atgccagtttgtcccg,
tgatcaaaaaaagcga, acggggtctgatcaag, gtacccccccttttgt, tggtacatggcctggt,
agacgtgattctggga, cacctaaaaaaaggca, ttcccagtctttagac, gaggtagggggggggt,
ccttcgctggcattct, cagatgaggtacgggc, cctagagtttagcatg, ggagatcccaatttag,
cgtagggtgcctgaca, gatagggaggtaataa, actcatggaattcttc, aagtcctatcacatct,
agactagtctagacaa, caagcctacagtactg, ggtaatatgaaagaag, gtaaattagggtaagt,
aaaaaacatgcgagca, ccgctcggaaatcata, ggggggacaggacgcg, ccgccctgcacccgcg,
gacataggccagtgca, agctacccccaccccg, gctctcacgcccacgg, gcggtaaaacagaaaa,
tcggtccgcatgcagc, cacatatccagagctc, gtgtgctactgtattc, ttttaatggagagcgt,
tgttaggtgttttgac, tacttagatatatcat, tgctaagtccccccaa, aagtaaatattcgaga,
gggatgtctccctatg, tccagtgtattatgaa, tctctcgctgcaacaa, ctaggtacatggtgcc,
cgtccgtcagcgtgtg, tgagggttgacggagc, gtatattcccattggc, agtcctacccttcact,
ttgggccttcagcagt, tattagtagattgggt, ctccagaaacatagat, gctgtatagtcctagg,
tcccccccattccctg, attaaaaaaagtacgt, cttgcgtcttcgtaga, ggtaattgttgttagc,
ctgtagggagcatgct, agatgtataccgcaca, ttgctgaaggggctgt, gatgtcatccaagtgc,
cccagcgaatttaaat, attgaacccccccacc, ctttacgcccaaaact, acaaaacgcagaccag,
ctaataatgatgggct, actgcctgttatactg, agaaggtccctccata, actatgctaagacata,
ctatgggcgtaggccc, ggccccccccccactc, atctcaatcacttagt, atgggggggataaaag,
gcaaacttaaagttta, ccctgttaggttatgg, cagaatcccttagtaa, tgggggggggactaca,
ttagagccccgaacag, catttacgatttgctt, gcgatgctgggctcgg, ttgatgattaccatgt,
tgcacgaaatttcttt, cagacgcctttgcggg, ccggggggggggggga, ctgtggcagagagcgg,
ggttacagattggctt, ttgctgtagatgcacc, ctggctgtaggggggt, agctattaggatgaaa,
cttcaggatgggccaa, ctagctaggtatctct, ccttctactggctcat, gagcgatggacactgt,
caccggggatgaccgt, tttattgcttggcaag, cggctaattttctgtc, cagcaacttagtaggc,
tcctgtagtgggattc, aggactcatgcagtaa, cagtgccactgtatcg, catgccccgtgtagtt,
caaaagttcgccaggt, ctaaagcagataccct, tactgaataatgtcat, ccaccgaggtgggccc,
tgactctctcgactga, tttcgtggggggtgcc, actactgtaatgacat, atactcaccttactgg,
cccttaatttatgaat, ggggttagccccctcc, ggctgaccacggctag, tggctaccacttaaac,
ggtgtgtttcacccat, gatcttatggttttga, gtggtgtgttaaatga, tatgggtatatggcaa,
attgtaatccagctgt, tgaaaatctcccggta, tcttctcccaggttaa, tcccacaagcgcccat,
tgctggggtcattggc, ggagccttgggctgcg, ttattatgttactgac, ttctctgtgaacccta,
ctaatgataaaatggg, ttaggggggggtagaa, tctgaaccttctgttg, acatcacaggggggag,
tcgtgctgattccact, gacttcttaaacgggt, atggtctggcagctta, cgggcgctttccagtc,
tcgtgtagaacgtaga, tcattcacttagagct, ctgccgcccattggga, cactaggtcaatggct,
tcgaggtctagttcat, tctacaaatccttgat, tgaataaccttaaact, agttgttgcacttaag,
ttccccccccaaagtt, tagtgacaaaaaaaac, gatgatgaggacaacc, gagcttcgacacggct,
ggccttcgggagataa, tcatacgcatctattt, atattggcacctctaa, gaggagctaagacatc,
gctactgaagagggtg, gcacccgtgcaggccg, gatttcggtgtggatg, aatgcgtggccatatt,
tcgtgatcctcctgca, tgaaacatagttccgc, tcgtcacagcagcatc, atcccagcacacttag,
gtgagacctgtgccgc, gctgcgagtccatcgg, agaatcaacaatctca, tcctgatattatactc,
cattcctttatctaaa, tggtaggactcagtcc, ctcgggggggggatgg, acttagtgcaacgctg,
aaatggtgcacttatc, atctatgcctggtgat, tcgaagccggaccttc, aaatgtccaacaccgt,
gaccttgtccacctac, ggcctggcttttttcc, gggacatatagcccat, ctgcgtgaaaaagcga,
ggatattttattgggt, tgacgggcgtgggatt, tcagtggaccccccca, tgttggtcttgggccc,
tacccaagggtaccat, agcacttcccccaacg, atgataacgctaaagg, atatggttaaaacccg,
ccaggacccgccgctc, cgtttgctttagggaa, tgtgagaatacggtgt, gtcattctcttgctgc,
agcctaaggtgcctga, caggtgggcggtcctt, gcgctttttttattat, tgtccggcgcgggcta,
ggtcttgaccttaggt, atcgtgctttttttcc, ctctagggggcaaaaa, agcacctgcacaacat,
cacccttactttatag, tacgtagtcattcttt, aatcttaacgtcacgc, accggaaactgtagtc,
cgggctaagccctcgc, ggggggggggtaagct, ggggggcatagtagca, gaacctaactttataa,
ggatgataagggggat, aaaacggacccaggct, gagacggctacggatg, tttacccccccaactt,
atagtctgacctgatg, ctcgttgagcacgggg, acagttcgacattaag, aacgegccccccccac,
cccatgcccccccccg, aagtgtgcgtctgcag, ctttctcctcgctctt, cctggacaacttggtt,
acgaaattccatctga, tgaattagaatggtcg, ggggggggttctattg, aggttttttagaaacc,
agttgcacacaaaagt, gctgggaacggatcct, tgtgataagatcctgt, gacacttgacaccata,
caacccaacacactta, gaaggcccataaggtc, tcttgcaaaaaaaggg, tccgggtcttgatgaa,
atacccctgaccccta, tctgtcagggtcaatc, tgatacttaatagaag, agaggttccttgattt,
tggcacctgttaatat, ggaacttggcctttag, agacatatactcacct, gtggcaggctagagct,
aggagtatgcttaggg, gtaaccctcttttcct, aacaaaggttaagaca, gaggtgggtatgcctc,
ttagctcagctgctac, tggtctgtgtcacgcg, gggcttagataaatgc, ccattccgcagacacg,
cattagcatggaacgt, ggagggtgaggtgata, ggtatgcttttgtaat, gtggctgaccacggct,
ttctcaatgacttatc, agctcttagggcaggc, gcctttttttaggcct, cgcactcttcctgttt,
tagggagttttgccta, attgggggggtgaatc, aggttctgacgccact, ggacccacacgggacc,
ttagcctacatcatgc, aagcagagatgattac, ggcgcccgctgcaatg, gtggatcctgtaccta,
aattaagccagtgtcg, acatccctgctaatga, actaggtggcaatagc, atgtgacctgtatgtg,
tgccgtgccggatccc, ggcacgtcccataccc, gcaaaaaaaacacgag, gctctccctatgtggg,
ccccacaattgtattg, cctagctctaccttaa, taggacgaaaattggt, ggagaaaacgggcttc,
cgtaccaaaaaaattc, gcttaggcggagaatt, ttgcctattgcctcac, accccctggtgcattg,
aatatctcagtgccgg, acgaggctactgaggg, tggcctccgaaagcgt, gttactgagcctgaaa,
tacgtctctccatttc, cgtaggataagttccc, cttaagctacacctaa, aatcaagttagcctca,
acactgagatggccag, catgacgaggtcctgc, agggcttagccttcag, aaatttcatgctcgtc,
ttctgttattgattcg, tagactgtgggatact, cctaggttaaataaca, gtgtgttcaggcgcaa,
actaagtttttgaact, ggtcttgatgtacttc, gcaagaccgaatttct, cgtagctcgaaggaag,
cggctattggccgaca, ccaagctattaatata, agtgccttttgctaga, agccaatgagcccttc,
ttaaatgccccggtac, tggttgtttcgttatc, atcacccccccaactg, tgtccacgttaaatca,
gcccctgagatagctt, agccctcgatttggct, gcgctatttctttaac, cactgagaggatggat,
gaaggaccccccccga, acggttttttttatcc, gacttgaaagtatggt, tcctaggaagtaaccg,
ttccccgagccttgcg, ccgagggggggggaag, aaaggttatccacaac, tttgaaggggggggca,
caaccgttgatcttgc, tgtacacagtttgcga, ctactggatattgtgc, cacatgtcgctggatc,
gatatggtgacaattg, catgtctggcccgttt, tggtatgggatttctc, ggacgggtgcgagtgg,
acaagacaccgatgat, tatgcatgcggttgat, gcaagtggtgtaatca, gaactctcaggtcccg,
gctacttcctgtctcg, ttagcgctgcgatgtc, caggaagagcgaggga, ggttgcatgattgtac,
tcacacccctgtattc, ttacaccccagccttg, gcctggctcagcgtat, tcctcggctccggctg,
tccgcatttttttcca, acgatagggccacttc, gatggagtcacgtaaa, gtcttcggggtcccgg,
cctctatcaaagatga, ctgataagatatgctg, ccaagaacaacggcct, gcagaatgtacttcct,
tagtcagagtggttat, cccaacctgttagagc, gaaacataatgattac, ccgtttgctgtcagtg,
gcatggttagtttgct, tttgtcccccccatgt, ggttgctggagttact, cctggttaattcttca,
ccaggttgcccaagtc, cctggacgagtgacct, tgtgtcttccggaggc, ttttgcacatttacac,
ctgcaaatgttgggtc, cagtgagcatagggat, caatcaataagataag, caggacccacacggga,
gacgtgagagcaggtt, tattcgtttgtactta, agtgttattgaatatc, gcaagtggcttgatga,
tttgggttcagcacca, aaaaacgggggggttg, ttactaaggcactgcc, atgagaaccattttct,
cctgcacccgcgtgcc, gaaaggtgcaaagtac, gagcatgccggacagt, tagaaaagaaggacga,
ttaggggggggggggg, ggaagttagacccaat, ttcgtgaacaaatgga, acgccggctaggccgc,
gagtctccacattgtg, cctcccccccattccc, cgagaactatgctggt, cgtgtggctgagagga,
cggacttttttttggc, gacaccgtgagcattg, atcttgtctgtctata, ttcacttgtattgata,
acacagattaagctag, tccactggtgatctgt, tccaagactccttagc, gagccctcggttggtt,
atagacctctagcaac, ccgtacccaccgcctc, aagggattatcaatta, tctgccatgctacctc,
tttatacgaattgtta, aacagaaggcttgaga, gggcccttgtgttgta, gcccctctatgccctg,
gcgacccactggcctt, cagggtaagcctaaat, tttagggggggaggga, gcaggcgggctttcag,
tttgacttacttgaga, caaccggttaggtcaa, tgtggggggtagactc, gaacttgttccctgct,
catataggagaagatg, agcatacgtcagcttt, gctgctgtgcgggcat, cagatggtgagctgtt,
tggttaccctgaacct, gcaaaaaacaagcgaa, acccctaaaaaagctg, gcccgggggtccccta,
ctaatgccactgagat, attaggtctgtgtaac, cgattttgacatataa, ccccctggtctgaagt,
agacacccaacgaggc, gcttaagcagagcaag, acggagagcatgccgg, gccgaccagaaaaatg,
cttctcatagagtata, gggggagcgcgtgagg, ctatcgaaacaaatta, tggggggggcggagtt,
ctcatataaaaaagct, taagccacaagacgag, cgcgtcaggctgttct, tctcctgcccaaaagg,
cgaacccccccccttt, tctaccaacttagcct, cttccgccactagatg, tgcaatggggtgaacc,
cactcggcggcagctg, tccttggggggggaca, ggtctgacgctgctaa, aggggtctaatgacat,
cccgtagaagctgttt, atatgggtaaggtggc, tcaagctctccaaatc, ggccagcgtcggcccc,
tctgtccccctagtgc, atgtagacaacccctg, ggctttagtctgtgaa, ttacatgcgtctgtaa,
cattgcccggatcagg, ttgcactcaactatta, acctcaggtaatgtac, ttcttcactttccggt,
aggcgtaggttccagt, cttctggacacttatc, ccaatctgtggtaaca, ctgttttataccctcc,
gccctgaaataacgcc, tcagacgccttgcgtt, gggtacaatcccccca, atacaccagttcacct,
tgggggcttggggcgc, acgaaaaaaaagggca, ctgggcgacaggtcga, ataccaagaaaccaga,
ctttacaggttcagga, ccgggtcttgatgaag, tttaccccgccccccc, agtacaaaaaaaacgg,
accagggtacaggggc, gccaagatcccggcat, taggtgctctgtaacc, tacccaagttctctgg,
cgtggacatagaattc, ctgctgaccccttgtt, caagttctgacatctt, aaattgaggtcaatag,
gcagttatccggtaga, tgggacagctaaaatt, tgggggggggaagagt, attatggtgtgtgtcc,
atacttttaagccctt, atgcttacccctaagt, ttttgcaaattgcgca, ccttatcccaaaagca,
ccacagctaggctaag, tccccttctcacatat, aactagttctagtatt, tgaactagtaggcaac,
ggcagggccgcgttgc, ggatggcacgtgttat, gaactgcataattatg, tgatgcttgcgagcat,
agcaggtcgcaggagt, caggcgcacaatacgc, aggtcctagctctcaa, tctccctcattatagt,
ccccatgccgcctggg, gagggctgtaactttt, atggcacatcaaggtg, taggttgtcagatgaa,
cttagctgtcctgaaa, agtggattggatgacg, actctgcaggacaaca, cgaaaaaaaagacctc,
tggttacacccgctga, cgctcttcgaggtgct, cccccgaaaaaactaa, ctttcttccgtgactc,
tgaagcagtgttcctc, gcacttatgtaggtca, agaacctagataatta, ggcaaaaaaacggtaa,
tggcgggagcacccct, aacaaaacgtacagtg, cgggccccatggggtg, atgaggatataccact,
gacaccaaccccccca, gtcaactcttttcact, tccctacggtatcacc, acgaaaaaaaaaccga,
gtgcgcttcagagaag, tcccaaacttgtaatc, gaccacatccatgttc, cttggtggcaaagggg,
tgcatttggcccctaa, tacaatagagagtaca, gcttaacgcatgcaaa, aaccccgaggtatcaa,
gccacttatagataat, tttttagtctatgtac, ttgtcagcgtgtggct, agctgacgctgctcca,
ggtctggcagctttga, acctctatagataaat, gggtaaaaaaaggcac, gggaggggaggctacg,
atctgagattacacct, agcaaaaaagtggatc, agggcactatttgact, cccattcactgaaatt,
ctcttcctgcacgggc, gtccttcttccccctt, cttaaaaaaaacgctc, ctcagttctgcaatac,
cctcggctccggctgc, atgtccttacaggatt, ccaggtgtttaacttt, tcagccagctagggga,
ttgtccaagaactata, gcatgccccttatgac, tgtgggggggggctgc, gtaaatatgcatgcgg,
ttgcaatatgccttgc, caggatgctgagccat, gtgcgtgaggagaacc, caagccaagtgcggga,
aaccacgtctgtaata, cttctgtgtcgcccac, gttgggacattaatgc, atcaagtccggcaggc,
ctgtcattccccccta, tgaaaccttgagctag, tagttaactaatggca, ctaagggggtgcagga,
cgcttttttttaacgt, gaaaccgagggacaat, agagtcaattaaatgt, tggtcaaatgttagag,
cttttgggtatcctca, tggaaaagcggtcctg, cctaacaggacttaat, attacatacagggtca,
ctactttcacacctta, tgtaggcatgtagtgc, gttatcgacaaggggc, cggctgtaggcggcgg,
gttatgaagtagagcc, acaggatcagcacggg, tggagtttgtcctgct, gtgtgtgttacggggc,
tgctcctccacatagg, cgaaattccgtctaca, acgaaagttaaacaac, ataaaacataaacgct,
aatctttactcgcctc, ccggcatccttgaggc, ccaacctccgcatggt, ctttagggtaggaaga,
gccacaggagcctgtt, tgtaggaaaaacgcct, tatactaattgctaca, cccggaccccgagggg,
agagatcaattgtcaa, gatccttatagatgaa, gtcagcaacccagccc, ccctgagtacgaatgc,
cggccactgcggctta, agatgcactccacgtt, ctttatgaggagcgtg, cttaactagggctggg,
ggcagttgtcccccct, ttaactaggctgcatc, tgcccggactcacact, ccttatggaatgatgc,
aacgtgggggtctccc, cactttttttatcata, cgggggggcccgaagt, cctatgaggtaaagcc,
gggtgtaagaataatt, gcgcccgctcagcgcc, cggggggaggggtacc, tccaccaaacgacaag,
tgggtaacctgttcaa, ccttccgtcttacaca, gcattacttgagcatt, acttatagtctccata,
cagttccctatgccca, tgcctgggctacaggc, tagtgcgatcacctga, tgattttgcgctgact,
tctaaatactctcctc, agggagactcctctcg, caactttgcaacagca, ggattataggcgtgtt,
cccgcattcatgctct, ctcaatctgggggggg, gtgggttgtcctttgc, tccccctctggttggt,
aataacactctgctta, cactgctcaagtacac, ggtcccgcgaggccta, gacgatgttcccaagt,
gagtctggaggcgccg, cgtagtcggcgtgcca, catttttcgcataaaa, ttctactgcccccccc,
tttcgaaatcatgctt, cataaacttatgctca, cacctattacactagt, agtctactgactctaa,
gaggtgactaattgaa, acagccattaggtagg, agggcccatgcacgtt, gctgagcgtgcagtga,
caggtgctgttcaatt, atggtattccaaccga, cagtgagactggattg, cctccgcacccgccgt,
tatggttacaacaata, cgggggggcctttggc, cctcgccttgagtgtt, aatgagagagcgagcc,
gctactcagcttaggc, tcactcgctcaaccaa, cagcctcgaaatctca, gcttctgggcgtgagc,
cactgagcctagatcc, taaaccagacgaaaaa, agagcggccgccgagc, tttttacactcgaaag,
cacttggattgtggcc, ggcaagagaattcact, ttcagacgccttgcgt, tgacaggtctttactg,
atatagtgaagtacct, acaagaggatttgact, aggttattcacccgcc, gatcgtttgtctcctt,
atcaactgctggtaat, gcccagaggtgtcagt, tgttttgggcccgctt, caaatcaagacccaat,
gccatcctgatatgat, agttaaaaaaagcctg, aaagagtaaaatccac, ggcttaggcaggacga,
gcatctgtagcatatg, tgcgcgggacttccag, cggtctgggtttgtgt, agttttgaggacaatt,
actttcattagtccgg, gtgacgagaggtgatt, cggcaaggggggatat, ggctcggcggctggac,
ccagtctgacattgct, gataattggcaagatc, gcagttctaatcctgc, tcgccacttttttaaa,
gcagatagcttcatag, atcacggtaaccgatt, tcatcggaaagtttga, agcgagcaagaggtag,
tcctcctcgtgagttc, gaaccacagcttatat, agcggcaggtcttttt, ggttaaaatcaagagg,
aaaaactgccgggtga, cacccccccatgaaaa, cccatatggatttttt, catacccagggtcaga,
agatggtagcccagtt, gctgaaaggccagcct, ctaaggggaagtgcag, ttagctgcagccggag,
gagggggggttaaatt, tccccatagaggggga, ttaacgcccattttat, gagtaagaccaactga,
atgttaaggaccgtct, tcttgtatttggtaca, agcgttcagaatctct, ggtcaggagagcgagc,
acctcatacataagag, ttgtcttactacttac, tccgaggaggttacat, aaggtgccatgtcaca,
ttaagtaagcatctcc, gtagtggcctttttaa, ctgcctttgatgtagg, gaatgagagagcgagc,
cttgtggcagggcacg, ccgggaaagatggggg, acatacctgggactgt, tccacccccccccaag,
agctataccctgctcc, aaactccatctcgcaa, agttatttccttaaac, actaaactcccatagc,
cgacccctttttttta, catgtccaattagaaa, aaggctcggaagggaa, gtggtctgttgattct,
tatcttgacactctat, cagaatcatggcacta, gagaaaggctaaccag, tagagtcttaactgtg,
caacctttatgttagt, gacagtgctctaatta, agagtattacccatat, ccacactttagcttta,
aggctctgtcctcgct, ctgcccaataattgaa, aagtgaaaaactatcc, caatcagaattaagga,
accaggctggctaggc, ctaatccctcattaca, caactccagattcgcc, gtccctggaccttcgg,
gcaatctgtttgccat, aataacatcatgtctg, cgttatctcagctgac, tccacggcttaaagct,
aggcctaggggcctcg, caattatcctttcaac, cgaaaaattggcagtg, atggctgagggttatt,
catcttctccggccca, atgtctactggtttta, cggcctacacttaggg, tactgttatatgttga,
aaaatactgaccgaaa, tgctagacttatagtc, tgggcagtatagggtg, tttctctattacggat,
atcctttacaccatgg, atttacaacctaagta, tatctcggaattcttt, tcagcccttaggtgat,
aacctgggggggtcat, aggtgtaagctactca, gtgtggggattggagg, gtagtctctgcgaaag,
ttatagctaactatga, ctccatacagagcccc, ttagggcggaacaaat, aacgcaaaattatgaa,
cttgccggtcgtgcgt, atgatagagaacgggg, tggagatcttagaacg, gctccctacaacattt,
aatcgattttttttgg, gtcaaatactagccct, tcaggtgataatactt, cttatagaccttccgg,
aaacggtaaaattaaa, aaaccatcccactgct, ggtgtctctgaggttc, aataggttaagtacaa,
tagcataagagagtta, cgacagttagactgtc, tcacgacatcagcggt, taagagcccccccctc,
gtgctgggccactcac, tgactegcgccactac, caagagtattacccat, ctccgttgtcatgtga,
caagacaccgatgatg, ttcaaatgcatcatag, caagaaggcccatgtt, caccggaaaaaaaaga,
ctactggctcatcctg, ttagctcaagcgctcc, tctttgccggaaattt, gtctacaagtcctctt,
gctggggtacccccag, ttgcgtttttttttac, acgcttttagtttttc, agatttccaccaccac,
ccgcggcagcgggagg, gctggtaaaagaattc, gatatgcagattacag, agcctgacgtggttgt,
tattatgcaacacttc, aatgatcaagatttct, ctaaaaaaatgcgtct, ttgggccggataagga,
ctctctctcgctgcaa, agtaattgaactagta, ttgaacggaaaatata, cttgcaccatgtgcga,
tgattttttttagcgt, tatacaccgggcctgc, ctcacccagcgaaatt, ggcgttcagggactgc,
gctgggctcggccgac, agcctgtaactactgg, gtcggttcacataaaa, acatttcagggtcata,
gtacataggtattagg, gtggtggccccacacc, acttgcgaggcctgag, tgtagtgtagtgagtc,
agttaggacatcaggg, acatttatacgtgagt, gatctgacctgcctgt, ttcgtcttgggagaga,
aaggcttaggcagctc, aaactgtgcgaaatga, aactggagtggttaac, ggatgcggcagcccct,
cacctcttacacaaca, aatggactctcccccg, atggaaccacagctta, cggggatcctcagagc,
taaacacgaggttttg, atggagggtgcaactg, ctgtgcttcttaggta, gggaaaagggtaattc,
attctgggccactctt, ctttttttccgccgcc, gacacccaacgaggct, ttccggtaaactagtt,
ttgctctcacagctca, actaacagtgatatgg, ccatcgggaaggcagc, ttcgtgtctccatggg,
ccagcatctagaagtt, ctgggtagcctttaca, tgccacggagtagggg, gactaggagctcaaca,
catcctggtctggatc, atataacctactgtag, ggaacacaactgggga, ggatgctgacccccta,
ccttctcgtttggcca, tcctgttggatctatt, gttggagtttgctcta, agattagtctaaaagg,
agatgtacaaatctgt, atataggttaacttgg, ggtctacaatctgctt, agaaaaggaatgacgc,
tggatgaggggggggt, agacgacaagagatcc, ggatggctagacccat, taacatatggggatag,
atcccttttgcaacac, gagttatggtgggctt, gagggggggcgcccat, cttgtctgtactcccc,
gagtgccttgggtgtg, cgtcttttttttgtgt, taactgttttttttga, aaactagctaagagcc,
tattgcttcgctgagc, aagtctctgggatagt, acccctcggtcctaag, ccctgtatactgcaca,
tccatactcagtaaag, gtattggaccttgtcc, tcttttgttgacgaaa, aaaaattgaggccagt,
gatttgagacggaagt, gccacttacagtggaa, atatgtggtttctagg, ctgcctgccccccccg,
tgtgaggcctcccttt, cgtctggaactgggta, ggaataacgggggatg, ggttgactaaaagact,
tgttacggggcagatg, cttatccctgaactta, agccggccactgaagc, gactcgggggggggat,
ggctgcgttgcactcc, tcagctggagaaggct, tttattgcccctgtcc, caacgcattgctttat,
atttaccttagcttcg, ggcccagcacttttgc, cggcggagaaccttgt, cttgacgaagcagtta,
actagagcaacttaat, atagtcaagttatgat, gaagtgacgatacgcg, ctacggggaccgagca,
gcgtagcccggctatg, tataccacttctggtt, cctattgcctgcgcag, caagacataggcttgt,
agcgatggtgaagtgg, gtctttggctgcccgt, ccacctgttctatgta, aacgaagaataagaag,
ctgcagttatccggta, tccgatttgtcaatct, ggagttctagacctcc, cccagtcgtggtgtct,
cctctccgtgagagtg, atgaacatccttaggg, cctgccgccgctgtgc, tgctttttttgggcga,
tgcttcaaatacgtgt, tggaatcgaggtgggc, agataaacactaaccc, ccatctcggtttcggc,
ctcacggaaaactccg, agaaaaaaccgcagct, ccagggcaacatggtt, caatggtgagtctttc,
catatcatggtggctg, ggcgtggccaatatgg, gacaccttacttaatt, ttatcactcctgaaca,
gaaactagctaagagc, acgtttcatctacact, ggcattgtggcacgag, acatgtcttggccatt,
aggacggcccgcgcta, ccgccgccagcgctcg, aaaacggggggggaat, ctgggaacatctcgaa,
ctttaggtacaggtaa, tgtaaaaaaaatgcgt, caatctttactcgcct, atggcccccgccccca,
ggacacagattaagct, ttggttttatatggga, caacagctcgggggcg, tctaggagtatctttc,
gataagagttcttgga, gttttgcaaaaggaga, gtgggggggaaaaact, aagtaatcttcaggga,
gatagctgagttggat, gatgcctactctcgtg, tcctcactcggcggca, gtggacactaagtgac,
acgttgtcactgtttc, cctttcccccccccat, agtctcttttgggtga, tgttggagagtaaagg,
ccagttgttagttgct, caccgtgagcattggg, caatctacctatctta, ttataggtgcccttcc,
tcacgctgtgaggttt, ttggcttcacaactgc, ctgtgaagggatggct, ggtccttttgtggtct,
aatctgtgcggccttt, actatctcttttcgca, tgctgtgctgttcacc, ataaccctctgtgata,
cattgctcccccccgg, ttcacaccatcacatg, ttggcccccagttcac, acctctttcataactt,
tctggcagcttaagtt, ggttcagcgcattcag, tcagactaactgagct, ctcgcccagccgaatg,
ccactttcgtgggggg, agggaacccccccttc, gggaaaatctgtctga, aaagcaacagagtcgg,
atgatgtcccgacagg, ctcgatgtcctgaact, ttataagttagaatct, gcatactgccactgtt,
aactccactgcataga, gtccgaacttcctgct, tactaggccgggcttg, gggctctcatccatgt,
tctttatgactgatgc, ctctgtctccccccga, gcaagcaccccccaca, tactgcaagtgccagt,
tatgcatttctcggat, caacttctcgaggcaa, tgtggagggggggcaa, tgatgaatcaacgact,
ttgcagcattgatagc, gcgtcttcgtagatcc, ggtgactaccaggtgc, tggctcgtctggagtc,
ttacaatatttagggg, ctgtcccggggaccac, tacctcccccccccag, ggatgccgaggtcaca,
atgacctcgtggtccg, tgactcatcacttagg, gaagtgggaggcttag, atagactttgtcaagt,
cgtgctttccggggca, gggcgatctgaggatg, cttagttcactgtctt, caattggcacctgtta,
gctggaggggttaatg, gtcagttccctccgct, cagggtaacaagagag, catgctgagacgatgt,
caaaaaaggaaggccg, actcatgcagtaagcc, aagcaaattgagcagt, aagagcggtgagtcat,
cgtttggacagaaatt, tggacaattagcgcct, gggcctggccctatgg, ccactcccaacgtcac,
tttcctgtacgtatca, acgcaggctttccaac, ggccgcccccccccat, ttgcgcattttctata,
tggattggtgttgact, gcgaaactctctcaga, cgttgcttatgataaa, gacatgtcagtctata,
cacaaagtcttcaagg, aaattatatattcgca, gaggcaaaaaaaggct, tcaagctatcttcgtg,
agacttaggcagtaga, gcctgctatgaggagt, cgtattcattcttccc, cactggtgatgtgtcc,
tggtattacaagtacc, ggtccccgtctgtgac, tggttagaggctatct, gcatctcctgatcacc,
gatagatcgtttttta, ccagacccggataggg, tctaaactgatctgtt, ccttgaagataacact,
acacacgtaaagggga, tcaggaacagtcttgg, ggtggtttgctcttaa, ctataagggtgaacca,
tatttgatatacgtag, gtgctgctctacctta, ttttcactgtgcggcg, tgacattagttttacc,
attacgtttgtctgca, ccttctcaatttatgt, ccttagaggtaagagg, gataggtacatgtcaa,
tcaagtagcaagcaga, attctcagtatccaac, ctcggaagggaacagc, ttgtgtttgtgaggtc,
gaccctcgtacattct, cacttatacattgctc, gctatcctcgctgatg, aagtgcgcttttttta,
ggatagattatggctg, gccatccacaaattgg, agaaaaaactaactac, tcaccgtacccttttt,
aaaaacggggggtggt, ccgcttaggctggacg, tggcgatttttttggg, gcggggggggcgttac,
ctccacccaagaataa, ttgtcccactcacagg, tatcgacaaggggcag, tcaggcaaccatccgg,
tgaaacgggggagatt, tttcccgtttttttaa, gtacaattccccccaa, aattcagcaaacgtat,
tccgcctcaaatgaaa, cgttttcaccggaaac, ttacttgaagaattgg, tattctcctaaagagg,
aggtgttaatatgttt, gagaaaccccagagtt, gactctgcccgatcgc, gtttggagcaattgtt,
ccggagctcctcggct, aatcaagacacttata, ccggaatggtttagta, ataaaatgaggctgcg,
tgtcaccccccaagta, aaccccccggtgttgg, gatatgaatgggtact, gcaaaaaaaggacctt,
tttttggctttgcgtt, ttctgtttcgggggga, caaagcataagaggtt, tagtggaccaattgct,
tccagtgtctccgccc, gggagcttagcatgca, aggttccttgatttct, ctattatggacactgc,
gtcactccggtgaccc, ggggcctgcctgtttg, catttcatgtggaagc, ccaggggtaagctgca,
acctgaagctcggctt, tgtcccctacccgcat, tttagggggggtgaaa, ctaactactaatctta,
ccctccccccccgctc, aaaatctgctccagag, ccatgtggttaaaccc, gctgcgttgcgcactt,
gggggggggggggttc, gactctgcgatgcatc, aaaatgacatgtgtag, atactgcctaggctgt,
aacttactgcaaaacg, ctattggaaatggttg, aggggatgtctcccta, agcaactcactgggct,
tgcctccactttgaac, ccgagttattaggggc, ttccaggtagtcctga, cacacctgaaatacaa,
accctaaggctgcggc, tcagagttactacatg, tacccgccccccccga, atgggaacctttactt,
acttagatactgacac, tgtccaagaactatat, atattattggggaggc, ttctcactctcttcgt,
cacttgaaaaaaaacg, actaatgtatctgtaa, aggcccatagaggtat, gcataccctaaaaaga,
gtgtttattgaaccag, gttaggaaaaacctac, tagcatggggggggaa, gcacactgtgcaagtt,
gtctacgaaggagttg, tccggagtgagactcc, atcccagtcctttatc, tgttcgtcttctcatt,
tgggcaaaaagtctta, tgagttgggactaatg, tgtccaaaggaggttc, acactcacgtgatggc,
accgagttagactctg, ggcggagctaggctcc, cgtcttcgtagatcca, gtatacattgctaggg,
gatgcacccgcctgat, gctttgagccaggtaa, aatgccgcctgccagt, acattccggacgggca,
agttgctaccccccca, cgcactcttcttaacc, caatggttgggcacgg, catagggcaccaccat,
tgtcaaactgttggaa, tgtgagggggggtgtt, gacggctacggatgac, ctgaatgccgtgtcct,
ttcatagtttgatgag, gatcttgcctaaggtt, gtgcagtgcttctaag, aacccctatagtaata,
cgaggtcccagagtcc, aatacgtgaccgtttg, ttaggatgaagcctgg, aacgttctcatcttct,
ctatttagcaatcctg, cggggcaccttgtgcc, tgatttaaccacgtcc, ctacggtatcaccagg,
actgcaaatgttgggt, ccagcaaaaaaaaggc, gccagctctcacggca, acaggtaatgtaccta,
agggaggtccccatag, ttcatctggaaccgaa, ctcgccccctggacac, gccttttcacgctgac,
atagtagagtatacct, tagttgttagggatct, ccgcctggcagcttca, atatggggggggcact,
tactccttacttggac, actctctgtggacata, tttttcagagtcacga, tttcactgtgttatcg,
tgcacctagagccacc, tatcaagccaggtaca, atacccaacctagtga, ccgtagtccattagag,
tgcttatatgggcaga, gtggattggatgacgc, cgtgtgcacagacacg, agtctttgtctcgctg,
tcttaacatcgttaca, aactcacttaggacac, cggggccagcgtcggc, gaattcttacgggctt,
acttcctacttgacga, gcctttgtgggcagcc, tacctcacatccacca, aaaaatgacaaatcgc,
gggattagggaaagta, accagtttggcctcga, ccacgatacaattaaa, agtacataggagggat,
ctgtacaatttgagga, aaacgcgcagtattct, taattgagtgtcccta, ggtagggtggacccac,
ttaggtcggggatggg, gatcctccgacatcag, agtctgagtttttaga, gttgtgcagctggtcg,
tcccttcatgtgagta, tggttagacccccgtc, gaaagttttatcaatg, tgtgcaagtactgttt,
tgtgctgtcccgtggc, aaaaactagtggttga, ggatttcggtgtggat, gtgaagttatttatta,
ctgaatgtctagtgta, ccgaggcaggctgaat, gaataactgcttaaca, tgcgagtccatcgggg,
tgtaccaaggagagta, ctgccatgtgaccctt, tcccggagctcctcgg, aacctggttatgaccc,
gccaggtgattataaa, ggatgtgttacccgca, gcaaattcaagtcatc, ctgcagatagatgcta,
tgggtcagtattatcc, tgctctccctatgtgg, ggggtggttcagctga, cctgctctgtgtaggc,
aggtgatcgccctcag, cattatgttcaagcag, aaaatcgttcatagta, tgcagttggtcggtgt,
gccttgcgaccgccct, cattccaatatgtaag, gggtggtagtagccca, tttactcgcctcaacc,
gatgtacacgcccctt, ggaaggccgcggggta, aatatttgaggtagac, gaccatctttttaccc,
tccaatgaaccattgc, tgccccccccgaaaaa, tgcaattagtaactct, ctgcaccctcataatc,
tacctatccacccaga, agtgataccacaacta, ggtcaatcggtgcagc, tcctcactgatcccta,
ctgacttcgtttcgtt, atatgcaaaaaagcgt, gtcttacaggaactgc, ccgtcactgactcagt,
gctgattatgtacact, gctgaagaaatccccc, tggccaaacttaggac, gagaggtccccctctt,
caacctacgtgaaagt, caaaaaacgttagcac, gtgactgactttttgc, tttatcatggggggta,
gctaagccctcgctcc, tcacttaaaccggttt, ggcaatgaggtatcta, ggcccttgacatctgc,
cgtagtccgggtgcag, cttgcccccatgggct, cgttgcatggaagata, cacaatattgtccctc,
ttctgtgaagatgtat, gatggacctaagtttc, ggccatgcttcgcttc, catgtcacccccccga,
ttctttcctgcagtcg, aaatcgctaaaaaaat, acctaacactgcttcc, gacttccagccctttt,
gggatatcagtatcta, cttaggtcaagagtta, gtcgaaggcaggtgtt, gaaattgccccccttt,
tggctgcttattggtc, gtggaaacgagacatt, ttcgctgttatttcca, gggggagtaggttctg,
cgaagtagatatatca, caccgaggtgggccct, ggatgggcagtcctgc, taattttcgagaaaaa,
aaggttaggggaaagt, ctccagggaccgttat, acttactatggactcc, tcacgaacagtatagc,
agacgctggacccacg, gagggacggttattct, gccgtagtcggcgtgc, agcgctgagggagaag,
attttggcgagttata, caagacacctgcctag, ttaaaacttcacggtg, caagtggcttgatgag,
cctcgagcctcccaag, tgcgagttttggtgca, tgccttcttaagaggg, gttgggctgcacaccc,
agaacggcgtgacccc, cggaagtgacgatacg, agcgctcgcgccagca, tccagacacgtaaata,
gtttgttatccatatg, cacagggagacggcca, gggtcggtccgcatgc, atctaaagatggtgtt,
gccgggcgaagctcag, acttatagcagaaagg, tcgttgtttttagatg, attggttgactccacg,
ggaggccaaaagccgg, ttgttaagcctttcta, aggcgagtgttgtttt, taactcgtgtaaccca,
ggagattatggatcag, catagggcttcatggc, cctcgtgggctgctcc, taagccaagtgggacc,
ttgcgtacatatttgg, gacctggtgatgtacc, aagtgcacctccttct, cagatccatgtccatg,
ctctttgtgtcatagg, ctctattgtagttgtc, catgtcgctggatctc, cacacatctaagctac,
gcatcctctcacacag, ggcttccgtgagctga, ttatacacttttttgg, caatacctttagattt,
cgggcgggcttcttag, ctcctaaaaaatacga, acctgctcgtaaaagt, cggtgcacacagccag,
gaaaacccatcaccta, taggattctccttcac, taaatgccccggtacc, ctggtctatagtgcat,
acgacaagagatcctt, attgattttccacgcc, tacgtgcctgggaaac, atagggcaccaccata,
cataagattctccccc, tcctgatgcactaaat, aaggtgttgtagtcta, gagacttgagggtcgc,
atgaataaaagacgta, aaacggacccaggctc, agcgactcaacacata, cctggttacgaaatca,
aggtgacccccgaaaa, acgtataaattataaa, acgtgctggccattca, aactcagactttgaac,
actgtgtagaggtaga, aatccccccattatgg, agctttatgttacaag, aaacggcgcaccgtga,
ggcgtggactctggtc, gctgtgggccacttta, atcaaaagaatatacg, aggggcttgtctaggc,
cccccttcaaatggga, gagacatcataacccc, ctaagaatcctgcatg, atagtgctcccgtcaa,
cggaaacaggttattg, ctacttacaaatgatg, acatgggggggataaa, ttgatcttgcgaccaa,
gccgcccattgggagc, ttgtccccccccaaac, agccatttagcccggt, atactgttcgggttat,
acgcccaggggggggc, gcgcagctgtcggttt, gggaaacccagtaggc, cggaccccgaggggtg,
caccccattggtgttt, ctgagcctcgtgagta, gaaataatgttgtgca, ctcgcggccttggaac,
tcgagggctcagagca, cacgaagcctgtgtag, ccactcataatgctag, ttgtagcgacagggta,
gacaattcagacactt, cgtaaatgtggaccat, cgagtgacctcggggg, ggtagttgcttcactt,
ttgactaactgcctga, acagtgatgccatgtc, ttgttaagtattcacc, ccacccccccatgaaa,
gttggtccctaacccc, tgtaccgcaaaaaata, ataagcagaattagac, tgtcctgcttgcgagt,
aagagtataattgcac, tctccctagccaagac, cctctgcccggcactt, tgcaaaacggtttatg,
gacagacaagtgtact, atattaggagtttgat, tggtctggaaaccatg, taacctagaggttagg,
cttcgtatacatatag, ggggtcagatgcctac, aaggaccatagtgggt, ggctcgcacccattcc,
atgaatcaacgacttg, gtgctggcatccgtgt, tgataactagtaaagg, gtgtctacataacctg,
ggcctttagagacgac, tactttgcccaagagc, tcttatcgttttctct, atgctctatatcattg,
ccttagctttaggttc, gaataatcagctgctt, ttcaccggatgggctt, aagattagatttgggc,
acaagcttttgctcac, tttatcctacccccca, cctttggcttgtgtgg, tgactcgaaaggagtg,
cgaaatttttaacttg, gaagaccttacccaaa, cagtgacctcccactt, aagaactgtttgggca,
cattaagttatgcatg, tggtgtcccccttttt, gcacgaaaaaaagaga, gtgagttagactccgt,
tcggggggcggggatt, gcccggtctactattt, ccacgaaggcccagtg, ctaaaaaaaaacgatt,
tacaggagttatccag, aagggtgggagtatta, acctaaacgcttcaca, actgccaaacccacta,
ttgagctgaatagtgg, ctaagagaacaccaac, ataccctggataaagc, cttttaggccagcaca,
atgtgatgcgggttga, atgttttaacagctcg, tattacgctgagaaaa, gacttatctgtctctt,
atactcaaactttcga, gtatgaatcaatcaaa, acccctgacccctaat, ggggccagcgtcggcc,
aacttcaactcccctt, gtttaaagtgtcatac, ctgtggccttagcact, gaagtagagtcctctt,
ttctttctcggtgtga, gctatgcgttttcttg, gggtggccaaccaggc, tcctggttagcactta,
agttgcacccgtgcag, aaacggcaagggggga, tacatatgtgggtgct, caggtgccggaaaatt,
gttgtgactagggata, cagttataagccctgg, aatccccagccaatat, gtatcaccctcataaa,
tatgtggggggggtgc, atcaaataagtatccc, ggagtacttagcagag, agaccccccccatgtt,
tagtatctaattattg, tccagtgattcgggaa, gtcctaatttctcagc, atttgggaggggggga,
gtcttgtcggtggaaa, tctagagtttatgttg, agcccgataggcggag, cttgggggaaggtcac,
gctagataggttacat, acttcttatactaagg, gactgtattaaaggtt, agtctgactctgtgac,
attgtatcgtgaatat, tctccgacttgtcagt, agcttccccccccata, ggtttacattgggggt,
gctggaaatgagcgta, ccttaagcacaatgga, aaaaatttaggctcta, tgctaaattgaggtgg,
tgttgtccacaactgt, gctgcaatcatagatt, gcgaggcgctcgagta, ttgttatgtcctgggc,
atgaggactacgaaaa, aagtacccagggatgc, accttcctccgggtct, ggtcttgtcggtggaa,
ccagttgtggtgtcgg, cacgggggagcgcgtg, aggaaccccccacgca, tagccacctttatcac,
cctctgtgcgcctgtc, actggagttacaggct, gttccaccccggggct, tgctaaaaaaagcatg,
ctcaagcgctgcgcgc, tccagggaactgaccc, gtagctaagtttcatt, gcttaaactaaatgtc,
gcgctaggcggagaga, aaaaaattttttgtcg, cttacgggcttagttc, ccttctcgggaaaaaa,
ccatataattctgaac, gaggggggggtggcca, taaagttgaatagcat, ggcaggatatactctg,
gcaccacttggagaca, caaccaagaacaacgg, agtagtatattatagc, ggcttcttaggcgatc,
cccacaagcgcccatt, gttacacccgctgaag, tggaagctttttgacc, gtctttgtgaatcaac,
gctcctttaacatcaa, cgattttgttttatga, gcgagtccaacgtttt, cccggctgttctttat,
cccataagatgatcca, cgcacccttctggagc, aagagcgagggaaatg, aaaaaaactagtggtt,
tgtgggcagttagctg, cgctccgccttaagag, gagaggctcggggcta, tgaaacgaacatacct,
ctctgcccggcacttc, taagtcttaatcccag, ctctcggggttgttgt, aggatgctgttcacgc,
cgtgttatgtaataat, acctggatgggcgcgg, catattaggtgtctgt, cgggcacctgtaaccg,
gggctcttatggtcct, gtcagcaatgccgcct, gaacacgaaggtggag, ggcgggcttagtccaa,
gcacacacttatatga, aggtacctcggggggc, acttgctgaaggggct, agtgtaagctctacag,
taagacaagctacagc, agataagcccgggcaa, toggccagtcctctcc, cctaaaaactgctagt,
agtgagaatctagctc, gctaaaaaatgctagg, cacttcccgattgagg, cctcttacatgaatcc,
catattggtcgtatat, gcatcgtaaaaaaaag, ctgggcagttagactt, gtgggtcatgtgcatc,
tgagggcgtagaaggc, ggggtatgtctgtcag, gcacatcagaggataa, ttgccgctcacgtagg,
aggacactaaagtagc, cttagaggaagcgaga, ttatcctctcatgggg, accttagggcattgcc,
ccccttaatctgggtt, agttatcgacaagggg, ctctaactcgtcaagt, aaatgttttgttgcga,
aatccttttttgctac, ggagtctctcatagtt, gtgggggcacttcttt, aaactgattactcaac,
gtgatatgtgagatca, gcccccccccatggat, tgctgatgggggggaa, attaggtagcgcaata,
agatcttgcttgtcag, ctcacggtagtccagt, gggacttgcctcgtat, gactgctgttcccaca,
gaaatgagcgtagtga, gtctaaaaaaagccta, aaacctcgaacagcca, gtagatgcatgattac,
ttgtaaacatacgcat, atggtatgctgaaacc, caatcactaagacatc, caatctacattccgta,
cggcacttcccgtctg, tcgtcgggacgtcccc, cctgagttgacacggt, ctaggtcattttttac,
gactttgtctgggatt, acttttatgggcaccc, gtgtacggtacaccac, ctegtccccctatctg,
caaaaaaatatttcgt, tcaagctccgatgaat, acagaattgtgggaac, gtcccaggagaaattg,
cgtagatatgcttcat, ggacgtttggggctct, cttgagcatgggtgct, ccttttttttgtaacg,
atcttaagccgtgtgt, taccaagatttcggag, gcaagcacactagtac, agtcctgtggggccta,
ccacaagatgaccctc, agagagaattcgtgac, ttgtcccccccccaga, agagtgttttactgag,
gagggctaaaatcgga, aaagccctaagctcag, atctactgaaccatct, ggggggtacatcctta,
tgagtccttttcattg, cttgtctcatgataac, actaatcgacttttta, gcattatgaatgctgc,
aagctatcttcgtgcc, ttcctttgttgcaaca, cctgcgtggcagagga, cttagatggctatggt,
ggttaagcttacttat, cagaatcccacaagcg, tcttgggccctgtatg, atcccccccccctcta,
ctccaatatacaccgg, aatgcgatttgcctgt, acacgagttagactgt, gcttctcatactagtg,
gacttgtgtcctgatg, gcaggccagatctctg, ctgccgccgctgtgca, cttactatgcagtgag,
caattggaacacctga, cggttttaaatttgtg, cctgagagaagatgcc, ctttcgtggggggtgc,
tcccatatggggtggc, cctaattctgcagata, ctaccccccattacaa, caatttgtaaaccctt,
agttgcagttagtagc, acatcagagatggtag, aacccgtgatcttttt, ttaaaaacggggctat,
tttataatgatcccca, ttacccccccccgaaa, ttaaccgtatattata, tattctttcttcgtgc,
gcacattatgcacttg, agggccatttatgctt, ttagaacctataacgc, tggtgacatataggcc,
ccatgccagttacagt, tgcagctggcaagtcc, ctgtctggtcaacaga, gcagggttgtaatatt,
atcatacgcatctatt, cttttgggggggtatc, gtattaacaccgatag, cccacacgggaccaac,
gccgtgccttccggga, tgcaacctacgtgaaa, gccaatggaaagtagc, cccaaagtttttacaa,
gtgcaggagccactta, gataaggtccctgaat, gatatgttccatggaa, gttttctcgtagaaga,
tgtttaaatctgatcc, gaaccttgttctcccg, aattcgattaaagtag, ggcaaggctcggaagg,
gctgggtaggagtcag, gtcgtccaagtagctg, caccctccgcacccgc, agatggttaaaccaca,
tacattgctagggtgg, acatgtaccctgcaag, tatttctggggggggt, acctcatggacggggc,
ttaggtggagggcaat, aggtgtcgcttaaaaa, gacagcttatagcagc, cgaaaaaacatgccgt,
agatcagggggagaac, tgaaagcgaatgtgga, gctttacacaaaacta, ttccctgttgatgcca,
taaccctgaactagtg, tagggggggagcagaa, aatgcttgtgcacctc, cagcgcaggggtgggt,
attctctgcaattgtg, ttgaggtctactccag, ttgcgcagaaattcag, catgcaggatcgtgtg,
gattctacagctgtca, ctaaatgggacctaag, ttgagtatgattagat, ctgcctaatttccagg,
taacttctgggtgggg, ggtttatctttagtgg, acggagtctgacgctg, atgatgctagctgcgt,
gccgctccccccactt, ctagcctttaggcact, cttaagcccaattgaa, tgcacctccctgtatc,
tcccacggccggccca, tgacactccttcaaaa, gtctggacgactggag, ggtgctcttagcctcc,
gagcagctccgtaatt, cagctcgcttctgtcc, acctgcttcgctgctg, tctgtatgcaatacca,
gctgtacctacccatc, cgcgttccaccatgct, tgtttaatgggggggg, cacgtgccacgatggt,
caacacaagctgagca, aaagacatccgtagcc, cccccgaaaaaaagag, gccggtcttgtcggtg,
atttgggtcccccaac, tggagatcccaattta, tttataggtccctaat, gctcgggggcgaaaaa,
ggccattcgaaatttg, ttacaccagattagaa, gacaccttaagtgcac, aggagctggttactct,
cgtctgcagttagctt, cagtgcactatattga, tgcctgttgcttactg, acaacttacagtggaa,
agagttaaactacgtc, atactgtgtgtataag, aaggggggcatagtag, gttagaccctgtcaca,
catgggggtggccatt, ttttaagactcagggc, tgcagctctcaacttt, aatcaactgaagtccc,
gagacacggtttaacc, tcccaatactaagctt, actctttccggagcag, ttacccacgatgaacg,
ggcgccagagttagac, atatgttcttagtctt, tgtgcccgggccccat, atcaggggcacagtcc,
tccagcatgtgcacct, gcctattgccttaagc, ttcccccccaatttac, cctgcttgcgagtttt,
cggcacagtgtctgag, gactggtaacccatat, gcgtagctcgaaggaa, ctattacggataattt,
agacacatggcattcg, aatctcatccctatgc, tttgcagcggttgtaa, gatggagattaaggtg,
gagatagggaggtaat, aacggcaaaaaaaata, gtcttaccatgcatgg, tgggtaaaccttatac,
gggccggttccctggg, gtttcgttatctgtgg, taagcctcgctgatcc, gacaacatctacgttc,
cttttaggtagtaatg, ccaatggaggctaact, catcccatgcatcaag, ttaaggtgttgttgct,
agaccctaaaaaaagc, tcatatttaggcctga, tctgcgcatgccatgg, ttcaaaaaaactgggc,
acacccgcatccatgg, agtaggggctccacgg, aggtgtgttaaaccac, tcgaaaactaccttgc,
aattgactgatcataa, acacttcaataaaacg, gttaagggggggagag, tctttcctgcagtcgt,
tccattgcccggatca, tgccttttttttgggt, tcactgtcagcgtagg, ggcgaattgttttcct,
ctgtcttcgcggatga, ccaatcagccggatgt, aaagtcataatacgct, acagattatggcgtct,
cgaaaaaaaagttacg, gtgcatcgtattacat, ggactcatccacctta, tacccggaaggcttag,
aggacgctcccatctc, ctagctagagttgaag, ctccgtctctcaatgc, gtctccgactgttgga,
agacaccggggatgac, cccaaaacgtaatgtt, ggttcaatatactcga, accatttttccccgtg,
tgtatcccgaacactt, tgcgagggcggggccc, atgcccactaacaagt, gagacggagtctgacg,
tttatacccccccccg, taggcaggtcaaggcc, gaggggataagacctt, tgtttaaaaaaacgta,
gtccggttagagggaa, cgcaggcatgccgggg, ctaaacgcttcacagc, catagtgaccccgcaa,
cgtgtggatgagaagc, atgccacagtgacgct, tcctgaactgtatact, cggcccttaaaaaaag,
gagttgttttttttac, gcgaattaataaatac, gacctttagagggagc, cacacgccccccatgg,
ctgactcagattgcct, agctgaacttcaatta, ggcctttggcccggtg, ttcttctggttcacgc,
tagtggcccctctctc, tctgttttgtagacct, ctgatctaaacgcgct, ccgaaggcaagatggg,
ttaggcgggtgtatca, tcttatgtagtcagga, ggggttgtaatcctat, gccaccactacgcctg,
ggactcggtttttatt, cgaccgagactccttc, atcaaatgtaatatgc, cggaatggattacctg,
ccatatgtactggtat, tccagcaaatagattt, tcacccccgatacgag, attccaaatatgaggt,
ctctgcatgcaggtcg, ctcacccgctctagaa, ccacttatctgccctc, cgtcgtccccagggtt,
tccgagacatgccact, agctgcacactgaaca, tacgattcaacaatac, agggagtggaaacgca,
agactagttctatact, ctttggttagacccca, acactttgcaagaact, gggaaaaggttatcca,
atgttagcgaattaat, ttaaaaaaggcagtcc, gcgtttcctacaggag, acattctaaaagtgct,
tttgccccaatgtgat, cacgatgagattatat, ttgcagaccttatacc, ttttgggcttagagat,
gatgtgttacccgcac, gattgccacagtgact, atcagcgccccgtccg, cgctttggttttacat,
gctgattaaactctga, gaacaaggcagaaacg, tgcacactgtgcaagt, gtggccgattggtcct,
ggcccatagaatggag, atgtcaggtgactacc, acatagagacttttag, atctgctaaagagcta,
gaacccttgctactgc, gtcacccccccgacat, cacaggggctgctcac, agaggtggatagttaa,
gcaatgctgactcaaa, gtaacaaggttcctgc, cgtccaagtaaaaatg, aacgtgttcttattgg,
ttttcgggggggggac, ctgttccatttccggt, ataacataatcttgac, gtatctattctaatca,
ttgaaatgattcacta, acggtttctgttaaac, agaagatgattggggg, ctaggtgacatttttt,
caaataggcccccttt, ggattgaaaactttgc, tctgctatggatgcaa, aggtgaatacaccact,
agttttataggggtaa, gggtctccgactgttg, cgcgggacacatgtgc, acgagaattttttttg,
attgtcaccccccaag, tgaagttcatggattg, catactatgtcgaaaa, atctcgccaatgccca,
gcccgggccccatggg, agcgaattaataaata, gctggggacttcgatt, acaactggaagactag,
ggagtgagccaccgga, ccagtatatcaggtca, cgtcaagtggcttcag, accaaaccctattctc,
ttgcatcagaggtgaa, gagcttaggctgaaaa, gctgactttgattcta, gtacagggtttaacta,
gaacttactactctac, cgcccattgggagcgg, cgaaaatgttatagta, ctccgccctcattcag,
gcagtgtgcttgttta, ggttagagccacaaat, gctggcagaggtcata, ttgtcactgcaaccca,
ctatggatgcataaaa, ttacaaaagctgtcat, ggagatctcgccacag, cggttgataaaaggaa,
ggttacagcctgtctg, gatctagagcagcagg, gctcttttttttgggc, aggttaggggaaagtt,
ttttattccctacggt, tccctggcccttgtgg, gttcttgtccagcttc, gggcacacttactcct,
tactaaaaaaaacgtc, gactgtctctgagacc, atgcttacttaatgga, catccatcgtacagtg,
taaggaatatgcaagt, cgttaatttctcccat, caacattttatgcaat, gggattcccatactgg,
ggatgatttcctgtgg, ttcaaatgtggaccca, tagctgaagattcaag, cctaggatattatgca,
cattaatatcccaaca, gatgaggcctaaaaag, ttaacacatagcctcc, gctagaccccatctta,
gggggcatccccccca, aagcacattggtggga, gagggttagacaggag, aacctggggtgttaca,
aacctcactgattaga, ctgagggctgcttaac, ctgttttgtagacctt, gatgataatagactac,
aggcgctcctgacatc, aaatcgtcacagcagc, ttgccggcttagagga, ttgggcatcatagcta,
ctgttgctactctagt, aattattgacaactta, cagctaaacccccccc, tggccccccccggtgc,
tttaaacagattagcc, aaacacttatgcccca, tatgaaacgcaaaata, cgccccttgatatttt,
gacactgatatgttat, cttccatatctggagc, cgtgaggaggacctgt, tctgccccccccggct,
gccatcttctccggcc, acagccttgcactttt, ctaggatattgaagtc, tcaggttattcacccg,
gactcggtttttattt, acctgccttgcctcca, gtgtctacaccgcagt, aacttagcttaaaaag,
tgttcctgcaggttag, atagaagatgcctcaa, acagtaatctctctca, ggcacgcagctgccgt,
acacaccctcgcaagt, agacccaatcctacaa, cctaggaagtaaccgc, ggggactttcagtggg,
gatactagtgtaatgc, gtttaaatacgtcctt, gttttaagccgcaaag, ttgcacccgtgcaggc,
gttccacctcategtc, tccctctggggggggt, accctttgagatccat, tcagcaagtacgatgt,
caacctagggctgatt, cgtgtgttcaggcgca, cttgaggaggagcggg, ttagttccagctgagc,
atactagcaatgatag, aaactgaactggataa, ggtcgctggacacaga, gaatgggggggggcag,
taaccagtgcaccggt, tgtgggggtctccggg, ccgcatcccgctctgt, aactggacatccgccg,
tgggtgacccagtatg, aaactacagtgtagcg, gcccggactcacactg, gctgcaaccaatatac,
actgtagtgtccaata, ttactaatcgactttt, gtcaggcaaccatccg, gtcacacttgtaatgc,
gtgggttatctttgcg, tcataaaaggcccatg, aaaaagctacactagt, ccatatctaaccataa,
gataatagactaccag, ccgttgatcttgcgac, ctagtggagaaagaac, aaattatgcactgccc,
gattacactgcactta, ggactgctgttactac, ccaggatgactgcacg, gcatctgatgaactgg,
cttgactcaagtttac, gctgccttagcaggtg, tttccccgcaaagtct, catctaaacactttgc,
acagagctagtgacta, tatggacatggattaa, tcactttagcgacctc, gcctggactttgcacc,
ctgggtgcggcctggc, tgaaaactatgcctta, ctcccaccagtaaacg, gcaccaaaaaaggggc,
ctcaagtgctacagtt, acacttctgacagggg, ggccccccccggtgcc, accgaaatactgtgaa,
gtgctactgtactcct, ccccggtctccccctt, tgtgcgcttcagagaa, cagaggccataaaggc,
accgatttgcttaaga, gttagctcttactcta, atgcgcttgtccacac, aaatatcgtcaaatat,
atcacttagtaaaatc, ccgctcgcggccttgg, gagtgaagccccgata, gtgctggtgattatcc,
ctacatcctttaggaa, tctctcatgtaaatgg, gtgcgtgcatcttgcg, agttttttcccccccc,
acactggggaggacga, ggagagcggggttggt, ggctataggttggctg, acggatgcgggttctg,
caaacatcgatcactt, aagactatgtattctt, taatatcttaattgcc, gctgggctagcaatta,
attccccttttgaggt, aagacaccgtgagcat, gggaactggcgaccct, tccgtactgatgcttg,
cgctcccccccatatt, tgtatacaaatcgtca, ataaacctaatgttca, ggggagactgtaaact,
gttcttgtaactcaag, cgatgctcagggctgc, cccaacggactgtaaa, tttttttcgtttggtc,
accaattggtataata, cgtactgatgcttgcg, gcgagaaaaaggcgaa, agatgcccatgctagt,
tccccccaccggaaaa, gtctccggtttcatct, tagtctgtacaactgt, tgctcctagccatatg,
cgttctccaatttatc, ttcctaattggtttaa, tggacctggtaccgcc, caacttatagctaggc,
ggtcacccacatgtcc, ttaggatccctcatca, gtactcagctgtatga, ttcactgacactaggt,
cactgatccacctcta, tctcaaaaaaaacgca, aatgtcattgcgggtg, acgatacgcgagccca,
gacatatttctctgta, gagagtccgtgaagtg, cagtccacaatttggc, cgattttttttacatt,
caggggggggccagtt, cgcttggaaccctggc, gtatgtaactcctgac, gtaaagatggtgatac,
gtctgaattgctgggc, gggcaagtatcccaag, gtcaagtagttcagat, atgacatccccgacga,
atcgaaccccccccct, cgtatacatataggaa, tgcccatccttatttt, tactcaatgtacaagc,
ccaccttactccctgg, cgtatcagttggtgac, catctggactctttga, ccaaaactcttatgaa,
caagtggcaaggtctt, gtaaatcgtgtgctgt, gcatgacttatgtctc, agagacacttatgatg,
tactgctgacagggac, aattgataaagcggaa, acactattaggataaa, agggacatgcccaatt,
cttggaatactgtaca, taccagaggctgacta, gcctgccccaaacacc, caatgtaatatttacg,
ggtcagattagccagc, agatcatgtcacggga, acaacgcgcccccccc, gtaggcttcctgattc,
ggcgggaaggaactga, tttcgagtctccttta, gctattataaacgcaa, tcctcaattatgtgcc,
agagtctgaagtaaat, gatctaatattagatg, ttcctccaaatgccgg, tgggaggtgccggtgt,
gccagcgctcgcgcca, tctaccacctgagtct, tctccatctgccggcc, agtgggtgcttgcacc,
tccatgcggctttcca, cggctctcacacggtg, ccatcggggatcctca, tggatgaccccacaca,
cgggagagcggggttg, tctgaaaaaaaacggg, tgaattgaatagcctc, gctcgtcaaaaaaaat,
agtttaccaagttctg, ataccagtaccttgaa, cctatacccttttttt, gcattgccggatgagg,
gggaagatctcaataa, aaaacagggggggaaa, caatcaggccattacc, tacccccccagttaat,
gccctaccccaactaa, tggatatggtaagctc, atgtgcctgttgtcag, ctggtgtgtaggtctg,
cttatgactctcgggg, ttactgtgttatgata, tcatatcctttccagt, ctaagacttagagtat,
caacttagagattctg, ggcctcggcctttctc, ctatgctctcttggga, ggggctcttatggtcc,
gttaaaccccgtcact, aaagtgaaccccttag, atatgtgagggggggt, atttttttatctctcg,
acttgtacgatctcgt, gaggttttagagtctc, cagacgtgatcccaat, gttgctgcaaactgga,
ccgggggggctaacag, ctgccgggcttagggc, aatacagaagattatg, tgaaggtttcctgtga,
gaccaacgtgtagaaa, agtgaccctgtcttat, ccagactatctgatcc, ataccctgtgaatcat,
gtgttctacttactat, ccagattcgccacctt, tgcaccttgacctcgc, acagctccagaggact,
cctcgctggctcttag, tgaccctgaaggaacc, ctgcttggatcctagc, caatactctcagactt,
acgccggagccgttgg, aaaaaaaacgggctta, cacatagttctctagc, tttgtgtgttcgtcta,
gtgccgggcgaagctc, gcctacgaaagtgttg, ataaaaaactacacct, ccattgagcactatct,
acgttttaattgggcg, tcagcagcagggacaa, gcagtctagcatcaaa, agaacaacggcctccc,
acgaggtctgactgtg, ggtgcactggtgatgt, acactaggcatgcaaa, ggtagagccccaactt,
gtaaggcgggtggctc, gttacataggctggat, cccatacttgttagtt, tgatccttaggaaggt,
gccttatatgtatgac, ctattgtgccttatgg, tctgggtgtattcagg, ttggagtacattaagg,
gctcacagaccttagc, gacactcatcaagtat, atacttcgtatacata, cccgggagagtccggg,
tcatgttattgtgcca, gcgcacacaccaccgc, ggtttgtaatgcaatt, agggcgtctacacagc,
cagtcaacactgatcc, gctccagtttgggccg, tccagtttgcttagca, ccatggagtgatttaa,
gccgcgttttcaccgg, gtcttttttttggacc, ttgttttactaaacgt, cctcctgtgcctcggc,
gcaatagtgtaacagt, ggctcagatatgtaat, aggtgtccccccagaa, ctttaggtggccttct,
tgtacagggctaatcc, cagagattatggcagg, gttaaaccctcatgaa, gtgggcttaggatcct,
tcaacaggcttttaga, ccctttttttgtagct, cagaaatgagggatcg, aatggcgggggggaga,
cgggggggcccctgct, tatgcgcaacacatca, ctggtcaagatggtta, aaataaagcggactgt,
aggtgccggaaaattc, cccttgctactgcaag, agtccccctagggtcc, gattcgcccagcttct,
gggctgtcccgcgggg, ggccgggggcaacata, tcgggagcacctgtct, tactaatgcacgcaga,
cctattgccttaagca, tgtagctcagcccatt, gtcgttgaactgacta, cacagcaaggtagctt,
cttagtacgaatagta, tcaggtactccttaaa, acgctccgccttaaga, cactacgataggaaca,
ttttaccgcgttagct, aaaaatgtaatagcga, tgttatggtgacgggg, tatccagtccatcttt,
cagctgaaggcataac, atctgttatgctcttg, tcagatgcctacagtt, aggtaaggccagcagt,
caaaaatgatacgctc, tagtcgctttttttgg, ggggcacccccagtag, ggaaccttttttaatc,
gagaacctgctttatt, tgtttttcgtagtaac, atacgaggtaaatgac, gttgccttgtccttat,
ggctgcactctaactc, gttaccgtaagctcgg, ctccatggtggtgtca, gagctcctatctgggt,
tctccatctcggtttc, gtactaaggtcttttc, tattatagcagtctta, atgtagtgggaaagtg,
attcttacgaagagca, ccaatgttactgcact, acgtcgaaggcaggtg, gaagctcacccatttc,
gtcgttttctttggtt, gtagaggaatttactg, tgttatatcttacatg, gagatatcttagggag,
cactcaagctttgcac, tgcggactgtgcttta, ggctcactttgagttc, aagagtcacgcaccac,
ctgacacaatctcaat, gccccaccttaactta, gttgaaactaggcaat, aagtggtcctccttag,
aatgtaactcgtgtaa, tcagatggccggagta, ggctactagtctgtta, acccctactctgtgaa,
cctgtcctcttactgt, agcatattggccatgc, cccaacctccgcatgg, ggttcccccccttagt,
tcctaagtcccggatg, tacttatgaaacgcaa, cctctccgtccttaga, aaggttgttcagtaaa,
cgtaggttccagtgag, cgccactccaatcccg, agaagtttgtagtaaa, acaggttatacttatc,
atgcacccgcctgatg, cacccccccgacatgg, gagagccttagataac, attgtaggtgtaagct,
actagcctttgaacca, caagttacaaggaata, cgctggaaccacagtg, cagaggcttactaggt,
ttacccccattccttc, aagaatctcctgttca, ctaattgtcctgcttc, gtgattcgattttgac,
atgaagtcttgtaatg, ccgcgaggcctagggg, gggcttgcaagcttct, ccctacaattcatcta,
acctgtctccttcgaa, gataatcagctgcagt, tagctcaggatcttcg, cggtgggctccaccat,
aagtccctaggaacaa, cctctagtgcgatcac, atgctaagtcccccca, atggacacttcgtcct,
aatgatgtcccgacag, acacctgacctctctt, ggaccgcaggtctcag, tccaaggcataaatca,
aagtaacactcatgtt, agcaccctctccagtg, acgaaatcctctgtgg, ccgattagaatgacaa,
ttatctccttatgtgt, gacatttgtcagcgtg, ctgggtgtgaaataga, cgtctcacaaggctgt,
aacattgactaacagt, gcagcccctacggggc, ccccgttttgagtctg, gactggtgaaggggca,
tcgtataataaaaagt, tttgaggggggggact, cgggcgctctgatcta, agcaaggcttatccca,
acacgtgtgcatccca, ctcaatagaccacttt, cttttacgaaagcgaa, ccattgtgccccatcg,
acgctcccagatgagg, tgagtgaatctaatga, accttcaccctacatg, gtgttacttgccctat,
cttggtcatactaatc, ggactgtctttatctc, gactcggggtgtctag, tagaccccccccatgt,
cttcgtcgggacgtcc, tgcatatatccggcat, tccaaataatctcggt, tgaggaacccccttgc,
ctgtgtactgatgaac, gcttaagcgggaagat, tgcttaagacacccat, ctaactccctgaaaag,
ggataatccttactat, ctctttttttgcctag, gtcgagaaaggaagaa, tgtcccgggacgggtg,
tttttttggctttgcg, ggcgctattttttagc, agaaacgtaggtaata, gcaaggtacttcttct,
cgccctccaccatgcg, gggtcccccccttttt, tatcaaatgctaagtt, tgttacgatgtctata,
aaaactgccgggtgag, gcaaaaaaaacggtag, agcggtggggctaggc, gtgacaccagagctgt,
atgcctcggctggcaa, gagacggagattgcat, ttcctaggggcactaa, tttctaggccgaggcg,
gatgctactatcacca, agagcggtgagtcatc, ggtgaaaacctcatcc, gcgtgttccagcacac,
ttgatacgttggaatc, gcatgtcactgggggc, gatactctaggtccaa, gatcaccgtacccttt,
gagtccggccacgtgt, gcccctgtcgacaccc, ttccatccccgaaaaa, gctgttgcagtaagcc,
cacggggtttcttcct, cttcacatagtagtat, ggctcatactgaattg, tagaggagggggggct,
gaggccttacattcct, tgggtacaatctctcc, gaccatcttagccctt, tacttgacgaataggc,
caaacagtgcatcatc, gtacacagtttgcgat, agaccttccggatggg, ctgcttagcagtagag,
gccggttttttttttg, gacatatatccacact, gaacactctctgtgct, tgggcggtccttccgg,
gagcacgaaattgaaa, cgtaaatattgtatga, ttgctgatccgctgta, gtctaacaaaaccttg,
gggggggggaggttct, tcttctcgtctggatc, ttgcagccccagctcg, gaatcattgctggcgc,
cgctttgcacagcgtg, cagatgacttaacgcc, aggacccacacgggac, tgggccggataaggaa,
tggattagtctaaact, gacgacaagagatcct, gcatcaaaaaaaaggg, gacattccggacgggc,
cgccttgggggccggg, aagggaccagtcggtc, gataaaaggggggggg, tttcactctcgtctgt,
ttagccgtaaatcact, ctttgggttcagcacc, agaacctactgtgatg, catgaggggtgtcaaa,
tgagcccttaggctgt, ggggggggagtttctg, ggcaagtatcccaagc, gcattaggagacctcc,
tggcgggggggtactt, ctagctgagggggggc, ctcctcataaattaac, acggaagtacatttct,
ctacttatgccctata, agaacctgaggagacc, gggcccatagagtgta, gggtttttataaagta,
ttgcgtggtgctccaa, agcctgtacaatcaga, agtttagtaccacccc, cgatggtgtgatatcg,
agctagataggttaca, agatagggaggtaata, gaaaactttacgaaat, gcagatgaggtacggg,
gattagtaagtcctac, tgttgggaggagccga, gaggtgtagattgggg, ttttaggggggggtat,
ccagatccatgtccat, ctttggtacttgtaaa, ccaatgattgaattat, ggaccttcctctccgc,
ccgcattctcctgagt, gcctaaaaagggacca, cgtcctggggaagggg, tacttcgtatacatat,
gaaaagcggaacagag, cccatttagatgagtc, acattctttaccaatg, tagagaggggacaagc,
actaaacgtagattta, tgtctcgaaacacaaa, aaagtggttgccaaac, tacttaaaaaaaacgg,
tcttgtcgaccaggtt, tgttttccttctcggc, aggccaggaggcatac, tcacgttgtagagagg,
tctccccccccctcag, ccgacgcctcccagct, gatgccgaggtcacag, cgaggtctgactgtgt,
gataattagggaccca, tttcacttggtataac, actgtacatgtggctc, gctctcagagccacaa,
ctctccgagagaagct, acatgacaagactatg, atgagcgatgtttgcc, atgcgacaaaaaaagg,
caggcctttagagacg, gcacaaaaaaaacccg, actggggaagatagga, agtgccagtgcgactg,
tcacgcccagcacttg, gaagtacgatacctcc, gggttgtgggaggata, tgcaacccctagtctg,
ctggcgttatttacta, agagttaaccaaagag, ggacttcttaaacggg, tcgtttggtcacttct,
cttggtcttaaaacta, attattataacgatca, cgtaagtcattgaggc, tttaacagctcggtta,
cgggtttccattgcct, atgccataatggctaa, tataaacgcatatcta, ggtagcattacgaatg,
tagggttaaaaatagg, ctggaaaactcgtaag, cggccgggaagagtcg, tgttacctgaaagtcc,
ctcatctcctcgagtt, gtatgggaaaaaaagt, tctgggatttgtaggt, tccctaaactgtatca,
gacccccccacctcat, cagtacgtggttctga, atggggggataattac, ccttatacatgagatt,
gagggaggtacattac, gcgtaaaaaaatacag, atcggttgtcttcagc, tttggcaatcaccaac,
tattaccaatctgcat, ttgtaggggggggggc, cggtgatcctggtcct, cctgctgggaacggat,
tggtactcctactctc, cctccgtgggtgccct, gaggtttttttagaat, ttggttgtcaactatg,
cggaggtgggagtatt, ttcattagctcctggt, gaccttccggatgggc, tcgggcggcggagcta,
tgggcgctatttttta, agaccatcaagtacct, tcgctttttcccccct, ttcttcagtttcgtct,
ctgtgtgccgggcgaa, cagcgcctacttacac, ccctttgttacaatat, aaggggtcaaaaaaag,
tatgacactctcttag, acattgctaaggagac, tacttggaccctttta, taatttttgtcccgtt,
tgaggctcgcacccat, ccccgcacctccctca, ttactcgtgcctgtaa, acccttaggtagtcaa,
gtggcttcttatgtcc, ttaatgacccacattc, gtaggttgccatgatg, tttacacatgccccca,
ctaacaacagccagaa, ctgattatgcaaggcg, ttatgaactgctctcc, ctaatcatcctcttgt,
gcccctgtccattttt, tcttacacgcctgcat, gtaggtcccataatta, caaatagagtacctgt,
ggattcccctaatgta, cccaagcatgttagac, ggcccctaagataagg, acacataggaccaaaa,
aaaatggggggtatca, gcattccacttgtacg, ggtgtctccctgtaaa, cagtgggaaaatcgat,
tttaaaaaaaggcgga, aacgaaaaggacaagt, gggcagaatggcaccg, ttccgtctgcgaaaaa,
gcaacctccaagatag, cccaaggccatgatag, gctaactaggccgccg, agagcttttttaaatc,
aagttgcttaaagccc, caggaagatcgtttta, tcgcccttccgcactg, gtaacaggccactggc,
gtagctgtgagattat, tctcacccccgatacg, tagggggtgaaaggta, agtgtcatacctattc,
acgcagaggaccattg, ctaggaagtaaccgca, taaaagtaacctcatc, gccgtttctctcccct,
taccagttccacgtga, ttatgtgttagactga, ggctcagcgtatccca, gcacgtaaggagtcat,
tgcacccgcctgatgc, aatacagagggatgtt, agtgaccgctgtgaat, cctaagggtctcacac,
cgtcctcgagattcct, gaccgactcatcttct, ttcgttggggggggga, gcccccccccaaagtg,
ttagtgcaacgctgca, gaggaccgcaggtctc, gatccctaaccttgtc, taagaaattgtcctag,
ctcgatttctaaactt, aagcttttctcaagcg, ggggtttaacgtgtta, tgtgaagggggggagt,
cacaattagataagct, gaggggggtatatcca, gcttcactcgccccac, gtcggggggctgacgc,
ctcggatcagcccttg, ctggctgttgcgtgag, caatcgtgtataaatt, cctccttcgtgccttt,
tgaatctcctgttctg, cagacatgcagcttag, tgtacttcttgttggc, caaacctgacctcgga,
taggcttttcttaccc, ccatgagttttttaca, acccatcaccaatcca, atacctatccacccag,
tagctcacggtaaccg, ctctgttttcgcagct, cagttcgcccaagaca, gtagggggggggtcag,
ggaacatctcgaagct, gctttttttgggcgat, ttaagctatctacctg, tcgtggggggtggggc,
cccataagctcccttt, gatgatgtatcctgtt, ctaagctgggtcattg, tccgtggcttaaaact,
tttcttgtaatggttc, aataaacatcagatcc, tctcaattgtttgggc, cacatatgcaagagaa,
tcaaacaactaatgtc, ttagagctgcaaccat, atctgagctcgttgca, tcattcgcgtctgtgt,
aattagttcatagggg, agtgcaaggcatgata, tcccttctacaataaa, aaaaacaatcacggat,
ttgcccaagtccagcg, gcggttgataaaagga, cccaaactgtgctgtc, agcggccagccgtccc,
ccttaaaaaaaacggc, atctcagattaagttt, tttggtatagatctag, gcttcctcgctggcga,
gagagttgccacttgg, gcatactgactcttgc, ttaaaaaaggcttctc, aattgaactagtaggc,
acctgttaggattgcc, ggcggtggtgccagct, cgccccccgctatgcg, ttaggtctgacacgtt,
tagaagcaggaagcta, ataataaccttcacga, aaatactaatctctat, caactgagtggaatct,
ttgtaatgggggggtc, gatctaaaagtagagg, ggtcaacacaggaccc, ccgtcttacacattca,
ggaaatttgcgcagaa, ccaaacgtgtttacat, tgatcatattggagta, ttttacgtagagaaaa,
caagtggacgctaccg, atacgttttttttgta, ctcttacctagtaggc, attttcatacgtctgt,
gaagataaaatgtcgg, acactccacggggggt, gctaaaaactttagct, cataccattgatgcta,
gaaggggtagccgggt, ctccataagattggca, ccttaatagaaaggat, tacttataagtgggtc,
ccgtgcccccccccga, acagtgcatcttctca, acactagagttccttg, ctgcagtggcgcaata,
ttaagggtaaggggta, cgtatgaaaaaaagca, taatccaataacattg, agaactagctgtctgt,
tgtcagcttcacttag, atcttatgggggggtg, tcaccaaccttaatgc, gttccacgtgaaagat,
tttatggtgggggggg, cgtacatggtgacact, ggataaaatgcctcgg, tcctgaaggccttaca,
actttcaatgcagact, ttgcacttgtgcttga, cttcgagtacatcctc, tcttctaatgttcccc,
acacaacatggaatct, aaacggggggttctct, cctggcccccgaacta, gtaggttgtgaagtgg,
tgcatcagctgctcgt, tcatagatggttaaat, tataggtctgcttctt, tataacgtgtctgttt,
actagcagaattgatt, cctgatgtttagagtt, aagtgagctacacctt, gagattacggggatga,
acccaagcatgtgaaa, tgaggggggattgttc, gtttgtagcactgttg, aagggggggggtagct,
ccttgctggcatgttg, ccatcctgtttcgagg, atctgaggagcagtta, gtccacacctggttac,
aaggtcctaatgggat, acagagttagagtatg, cctttttccggctatt, taatagatgcagtctc,
cctgtctggtcaacag, agaatgtcgcaacctt, cgtcctctgtctatag, gctacggggaccgagc,
caagaagcagtggatc, ggacctgagatagggg, ccatgcgcctcccgct, ctggaggcgccgttcc,
ggatttttactatcat, aaagtacccccccttt, atctggaaccgaaaaa, cttagatttagagctg,
ggctgaaaaccgctta, ttagaagatctacatt, agcttgcagtgatgcg, aaaagactaatgaacg,
cggcagggccgcgttg, aaattgtaggggggga, ttccatctatgccacg, gcaagacctctggtct,
aatgcctaccaagttt, gcaaaacttatcggag, ggctgaacagcaatag, atgagagggtagtcac,
tgccgacagagtagat, aggaacttgtggtcaa, cctcacggggagaagc, aggctgaccgtaacag,
gggcctatcaatttgg, gattgtgcaactctgc, cgtgtcagagcctgca, taccatacaggctgaa,
aagaagatgactgcgg, attgcgattgaagcag, gattggcaacaataca, ccaagaagtaagaaga,
tgccgggcgaagctca, tcaaagtcctatgtag, agctcctatagttaac, tgcctgtacacgcatg,
cctcagtttgattatg, gtgatacatccggagt, acccccccccatacct, gatcagaggcacgtcc,
tttagattaactagaa, agcctggcaatgcggt, cttgcaatcctcccgc, ccccatagtgaccccg,
aagcctcaccttacat, agggcacttaatgtca, gggggcggagcgctga, cactctcttcgtttat,
tagttattccccccca, ctggattagttatact, cgccgcccttccaccc, tegccccctggacaca,
accccccccacatccg, ggtaaataatgtctca, gcttttttttaagagc, ttattcatatggaacc,
gccaagaatctctgtt, cccaggggatacgtgg, agagggagttgctgac, atgggtacttcttaaa,
gtccacttagtgtttt, gcttgccggtcgtgcg, ctatatcccgggggga, cccaaattcccgcttt,
ttggcgtgcactatta, tttggcccggtggccc, aaaatgacgtaattta, ctattattcaggcctt,
ccactcacaaccacta, acctcatagtccttaa, gtcatcaaaaacttgc, acgaagtccaaatcac,
tatcccgggggggccc, gacggattctcagttg, acttgtatctaaatct, agcgtgtaaaaaaaat,
acctcatgcatggaca, ctgttcccgcccgtgc, attagttcataggggg, ttgttcggatgatgga,
gtgcgtctgcagttag, caggtttcctagagat, ctggcctcttaggcca, taaaactgttgaaacg,
gatggagtcacttctc, tgggtaatctcaatta, tgtccggttagaggga, agttacggtggaggtg,
atcgttaagtgaaacc, atctcctgacaaataa, taatactgaccatact, gatgaactacgggaat,
taatgtctcatataga, ggacatctttaggtat, gatttttttaaaccga, gacgagctttgtaatt,
tagcttgctccaacct, ctgggagtctgccaat, gcgagtccatcgggga, gcaggggggggtctcc,
tgttcccctagaatta, gcggcagctaggctgc, tctgatgcctaattgt, gctaacatagggaata,
acactgtatcctctgt, aagtgctgcgttgcgc, ggttctattgccatgg, taagctaggggtgggt,
gcccgcccccccactt, ggcgcttggaaccctg, actgctgtagaccagg, ggcttcaaacaagcag,
tctaactaacaaaacc, ccatgagccgaaatcc, ggaactgtacacatgc, ggaccttgtccaccta,
aagtcttgaggactgg, taactcattcactgta, atcagtgagcccatgc, gagttactttttttgc,
gacacggcttctagca, ctgtggtattcataca, gtacctcatggattat, tggcgatgaggattac,
tgattctgcacactaa, ccataatctcatccat, ttgtcctcaactaggt, atcgacccacctgggc,
agggagggtccacgtt, ccgcctgccgccactc, cagggggaaggagtag, tcctgaaggtgatgca,
gccgctagcgcgggtg, agttccaaatccctgg, gtatgctctatggatt, actaatggacaggtgt,
gctcgaaggaagcccc, atcgtaaaaaaaatag, ttttaaagcgtgtacc, actaccttgttttgga,
gacaccataccatggt, taaccctctgtgatat, gtgagttgactcacac, aaaaaaggccgaaaag,
aaggatgctgaattgg, tctacaacaaactatc, tggtgaagctaaagta, ttttgagtctgaaacg,
attcaaaaggtgaaac, gagcgctgagggagaa, tagcccggttcccttc, cctcactgaatccagt,
ctaaatataactctcg, aataacgggggatgga, tgcttgtgctcttcaa, gggtactttttttaag,
gcggccttgccgcgcc, caaggtttttttatca, ttggagttgcatctga, catggctctcatatcc,
cataaggcaagctaac, actttgcccaagagca, ctccatgtctgacata, attttgctgatccgct,
agctgtgcacaatcca, gccaggagagtgcact, gtctgcagttagcttt, cggagcagaactatgt,
gaataagtggggtata, taatttatagacccac, gtgtccggagtttgat, ggccggcccagagaca,
attatgaaggcagact, gtgatggtcacactcc, gcatatggaagccagg, catgagagtggcgtga,
gctgctttcccctacg, gatagagcgggcgcca, acgttactaatggagc, cacaggcccgattttt,
cttgagactataacct, agctaggctccgcgaa, catcttatcggcaaac, gtaatgattaggtgaa,
acgtcaaaatgtctaa, gataggtttgggagtt, tcaaaaaaaaaacggc, tgaatacgatttttaa,
gagcactctaagggca, aaaaatgcacggtgtc, agagtccggccacgtg, ggagacagccctcgcc,
catggacggggcggct, gaatatagacagttgt, tacgctcattttttta, atccggcatgtagaaa,
tccatcagaattgcat, ctctctcgcctggcca, ggataaggacagggta, aatctcccctaagggg,
ccgataaaaatggtta, gacccgtcactgaact, cggtccgcatgcagct, gtccatgcgtgagctt,
agagaacttaccactg, gccttggcagggctaa, ccagtgcatgacatct, cgccttgcgttcgccc,
gtggttgcttcttccg, gctgaacttggaagtc, accatactatgtcgaa, aagtgaaagcgatttg,
accctttatgctaata, gtgacctcggggggcc, actaccccccgaaaaa, gtacccccccagttaa,
ctattgggagcggcaa, gaccgatgtttcatca, aagccaatgagccctt, gaaatggaaatcccgc,
agtgcgctttttttat, actttttttatgatcc, aacctcacacttctat, catagattaacgtaga,
cactgcatatcaaaaa, ctttggaacaccgcgc, tgcatttgggttaacg, tatcccagctatctga,
tataaaaaaaatctcg, cattagcaacctgtga, cctttctgttcgtaag, aagataaaatgtcgga,
gtgccggaacagttat, ctgcccatacacgaag, aaattcagcaaacgta, cccggacagcagccgc,
ttgtatcaggctttat, cccccgccagcgcatt, caaggggaccettagt, catcttcaccccgatt,
cgtatcccatgcccac, gctaaaaaaacctagc, ggcggtctgatcacct, caccctgccgaccttg,
aatggcccattagggc, tagtcttcataacagt, tagtcatagttcttac, tcaccgtagtccatta,
tgcaaaacttatcgga, cacttgcccatgtgga, actgtgacgtgtgcat, tgtaggtctgatttaa,
actatactatcctata, gtgtttgtcgctcaaa, aagaaaatatagttcg, ataaaaaaaagctccc,
attgcttcgctgagcc, gggtgccattgcacag, cagggtaggagggggt, aaggatcaccgtaccc,
cattagcagtcagcac, gcatggggggggaaat, ggaactggcgacccta, caggggggggtctcct,
ggtgaaacaccagcat, ctgggtgacacgagtt, gggcagccgtctatga, catgatgcaaatcact,
tgagcccgataggcgg, tggtgcccctgagtac, acaacgagatgatttt, ggagtctcgtgggtca,
cctacgaggtatttag, ggatatggtaagctca, gtctgccctcaacatt, atccatcataacttca,
tatacctgatagaact, gctgatcatgctcctg, ttaccattcctccact, gacgggcgtgggatta,
tctggtgtcccccttt, tgacatttgtcagcgt, tctggggggggcacta, aacgcgcagtattctg,
agagatgggtcattgt, tcaggccttttccaaa, gatcgctagggagatg, cggactcacactgcac,
tgaactcacccccagg, gtgctcatccccgggt, gggttgtctacaagat, tacaactattagaatt,
tctcctaacgttccct, gagccaagccttagtt, tggaggggggtgtaag, tgaaacaccatttaca,
ccgaggtgggcccttc, aaatgtctagggaggc, cttcacaactgctaga, ttgtcataataccata,
aaattctgagtacctg, ggctcgtaagtcattg, taccctagcatttact, aggtgagggggggggt,
agcagcggcgggagag, ttcacgtgtgttcagg, ttaagaatgtcgcaac, tggatcttaaattgtt,
tgcaaaaaggtctaac, tctgctggcaactgaa, tgcttaacgcatgcaa, gccgataaaaatggtt,
cgtggagccctgtatg, cttgcgaggcctgagc, tcttgtttaagtgctt, ccctggaacttcttct,
tagattcattgtatac, cgggggagcgcgtgag, gatacaggggcgggga, caaatattatggacca,
agacgctgaggagggg, atagggctggtctctg, agtacatggggagaac, gcttactgagagcggc,
atcctctatagtccac, tgcgactgtgtcatag, cagtaggtccataaaa, taacaagagacactgt,
aatgtcccttctccgt, agtctggaggcgccgt, gcaccaaacgtctgcc, cgactgatggcataga,
caatctctgtcactgg, tgacgaagcagttatt, aagtcccggatgtata, ttaaaccccatgtctc,
tctggtaacatgttta, tgtgtataagtcagac, accacagccccacttg, ctcttgatccgcccaa,
atttcacatagtctat, gcagattgtctttaag, tcactcagtctttgcc, cctctattgtagttgt,
gaagtgcacgtgtagt, ctcctgtccaatccgt, cgtgatcccaatatgt, gaaccagagaattgtg,
gctatgatgacatcgc, cacatccatagtatta, tttgaactggtgagag, tgtgtgccgggcgaag,
tgatcgccctcagagc, cacggggtctgatcaa, tttcagttcgcccaag, acgcattttttttagc,
tgttggttccttacaa, acctgtgccacccgtg, tcaccgatgcttagga, ctagtagtggtaaagg,
gggcattatggtcttc, aaccaggccgtggctg, aaattactctatctat, caccgcctccccccgc,
catgtccatggacact, gctaaaccccgtgtct, tttgtcaaactaacta, aatctgaccctgatgc,
gccatgtttatgatat, agagtttcttactaat, ctggggggggggaggt, acggttaaaccccttc,
ggcatgtgaccacgcc, aggagttctcgcgtga, tggctaagagttacag, tatccataatcagtta,
gaatagttacagtgta, atgcaataatcagacc, gttcactagagtataa, ataactcttagaaggg,
cacagacgtgagatga, caccttagtttactga, ggccttttttttgaac, gagagtacattactgt,
caccaccttggcctac, cattattaggagcatg, aaacgttttgtccgga, ggtcacaatgtgcagg,
cttggaaactaagaac, cttcgaggaggcagat, ccgccagcgctcgcgc, ctctctgaaggtctgt,
ggtaagtggctgcttt, tcctgaaccagcttga, attaggtttggaatta, tcagcttagagctatc,
ctgctatttgaccttg, cgccagcgcattaccg, gcttttttttaatcgc, gtttctaccccataca,
ctgtgaggaccatcag, aacaattaacgtgatt, caatgctgactcaaat, ttcttaaatacagact,
gctctctctcggtgcc, cccagctaggcaggtt, aaggtctcatcaggga, tgtgcgcgggcgcctg,
atagtcagaacttacc, aggactggccaatagg, cggggttgtggaatca, gggcctggaatgtatt,
gaagtggtcatttgta, atgtaatggttgtaaa, ccacactacccttatt, catcctctgtgacggc,
ccctgcacccgcgtgc, ggtgtacatgccagtc, cttactgagccctgga, aatgcttgccactatc,
ctagggggggagcaga, gcgttaaccacctcac, aaacagatagagctat, gcagtattcactatga,
tctaggaagtaccacg, catctctgaatggtat, ggtggcatacaactgg, atcatcatctatgctc,
gacccccccacctgtt, actttcgctgcctttt, gccctgcacccgcgtg, ctcttgcaaacactca,
ttacaggttagagaat, gaaatgtgaaaccaat, tgataggcaaaaggat, tagctgtgctatcttt,
tattcgaatttgagac, tttaaggccatacatc, tatcaaggctagaaga, tcttcctaaactgcta,
cttgctcggtgctggg, atgactttatgacacc, atccctgatatgcaac, acccccccctcactgc,
gagtcactatgcccga, tgaggggggtgttctg, tgtgatagtacattca, gctatgttagaccctt,
ttttgtctgttttcgg, gggcagttatgcaatc, tttatgcctggggctc, ttgatccagggtgttt,
gcccattctcttgtct, gtgaaggaggtgacct, tacttaactcattact, tgcataggcatgatca,
gccctggcaagagtgc, tatgttttttttacgc, ccttgacagcacatat, gccacctaacttagag,
ggcagcaaccctgata, catggcgatgaggatt, ccacccgtgagacctg, ggctcctgcactgctc,
ctttactccttcgtct, aacatttgcatctacc, agtgaagtacctctgt, aaaagtaggtgtgccc,
tggagggggggcaagg, agcacttatgtaggtc, gctgcgagggcggggc, ggcagataggaatcag,
cttacagaccgactgt, tggatctccttagttc, cgtcaggctgttctga, aagcttccccccccat,
gtctagagccatattg, aggtgtagatagctgg, agatggctccaactgt, caagaggacatctact,
tctggtcatgtctaat, gcccttagtcaagaaa, agtagctccaagcaac, acagtggctgctctta,
gcctcagtcgtccaag, gttgctcttctctagc, acgcatctgatgaact, caaacccattgcaaat,
ttctctcgatttcctg, cttactccctgctgaa, catataattgatctgc, tttaccatgtgggcga,
agtccggccacgtgtg, cagcggatcctttctc, aatctccaattcaatc, gtgttaagagatgcta,
ctagaaaacgatcttt, gagctctcatggatag, gtattgggggggtgaa, ccattctccttgtgtg,
ctttaccaatgtcctc, gcaaagcttatgtaag, taaccagaagttgaca, ggcatcccccccactc,
cctgaggtgacatcca, gacggcagaatccagg, gtgctgcgctgtcaat, cgcggaggcagaagtt,
caccttactccctggc, gggtattgcttcgctg, atgctatggcttttag, gtgaaagttatctagg,
acatggggggggcaga, ctagcggagatcgtgc, gtggattcttttagtg, accatgtagagtttct,
tagaactccatggcag, tggggtcctcgtgggc, ggccccagtatatcag, catccacacaaagggc,
tgggaccttcgggcag, gaatgctgttggctat, ttaggaggtcttcccg, agtcctactcggggct,
ttaccctagaactgaa, aaatagagcccttagc, ttacactcttgtggat, cctatagacgtataga,
acaacacgtgcttgag, aagagtcgctcctcac, tatcagtcccactcct, ttaagggtgacttcct,
tatagattaccccaga, cttacacgcctgcata, tgctaaggaatgtcat, tgagcagcatccctat,
tggcataaggattgct, taatggtgcggtgtca, tactatgatgaagata, taggatctaaaaaaag,
agcagaagtttggcgt, caactacacgcgagaa, tagaaaacttgactta, agacatggttctatct,
gaccatacggtcaaag, aattgtatgattaact, ctttagcccaggacat, aagttgacgtgcatat,
caagactctatggaac, gctaggtacagactga, ctgaagggaggcctcc, acccaggactcatatg,
cccctttttttggctc, cgaaaaaaaagggaga, gtttcattgataatag, caatggcagtgaacaa,
cagcctcgagaatcat, aaaagtggaccattgt, ctgcttttttttagga, ccggatccctgagtct,
cttactgatccccctt, tcgcgctgagaagctt, tgaggtgtgttagatg, atgccaccgccctggg,
ccggagcagaactatg, gctgtagcatataaca, attatgcggattaaaa, atgtacctaacaagct,
catggattctcctgag, ctgctacgcttgggtt, cggttagagggaaaaa, catttcaggcacagcc,
aggtgatggcagtgtt, aggcaatcatagctga, tggttttgctaaggtg, attagaaaatcaacct,
accttaaggtctattt, ataactggggctgcaa, tgtacaagacatatct, agagtaactatggtct,
cgcgggcgggttgaga, ggctaccactcccagc, tttatctaaggtatgg, gtgtccctgggccagc,
aatagcttgctccaac, gacgttttttaggcag, gatagcacaggctgtt, gtattatgaaacttgc,
tttgcttatgtgtccg, aagcctgatgctgctt, tggtgtgggctgagta, gagcatttgttatact,
gccaaagcgccttggt, gcaaaaaaccttccat, gcacccacggccacta, aaccctttttttgagt,
acaccttgcttcagtg, aatacaacgcgccccc, aactcccccccccttt, gttaaaccccgttcta,
ctgcgcagctgtcggt, atggaatggttgacat, cttcatcttatacaag, ctataaacaagaccga,
tagtagacccccccca, aaccttctcgtttggc, acgtttgtggattggt, ggcaaccttcgctgcc,
attccaaaaaaacggg, cgatggtcggctaata, aagtaccgtgtattaa, ggccctgccaaatgtt,
atcatgtcacgggact, ttttgctgatccgctg, ggaacacaggggtgcg, ggccagaaagcatacg,
atggggaaggtaacac, cctctggccccaccta, cagggcgaagtcacct, tcttttacctgtttac,
tttcattcgcgtctgt, tcgcacccattccaca, gctgaagagccttggg, tataactaaccataaa,
ctgggggggtcatcct, tcgttttactctttct, tgaactgttatctgcc, gccctcgatttggctg,
gtcagaaggtaaggac, cgattcacatgagttt, ttctccccaattagcc, tttcatacattttcgc,
taaaccatgtcacata, atcatagtttgttgag, acagatgaggtaattc, gtgtagcaattatgta,
tttctaacctaagagg, ctgcttgacgagtgcc, atggaaaacggggctt, agcagcgattgtagga,
caatatcctaaagtct, tatacgaaaaaaaagc, gtggtaggactcagtc, ggaattgggtatgcac,
cctcagtgaatcttta, cttgtttgccctgacc, atcgtcttagattatg, ggcaagatgctccccc,
ctgctcaatgtgtatt, tgtatagtattcgttg, attgtttcacacacgt, ctcaatcagagagatg,
tagcattttaccccgc, atctttagtggtgcag, gttcatgggtgcatca, acatatctacaggtga,
gctctaaaaaagatct, ccttgccgcgcgccgg, cgctgctttatgaact, tgtaatgataacctaa,
tgggccttcagcattt, gagaacccttagtggt, ctgtgccaaaaaagga, actgggcagggggggg,
cttagtcttaacggat, aggcggatcgccttag, tctcatagtcatttag, tgcgaaaatataaaat,
agctccgatgaataag, gtcctcaattaaaagg, cgccgcgggacacatg, atggactctcccccgg,
tggtatcctagctaat, taatttatcccccctg, ggcattagaatcgttt, ccttcctttttttaag,
gccctattagccaggc, aagaaacccatctgat, gcttactgtcactata, ctttatgtgctggcaa,
tggccgattggtcctt, ttcgttttgtaattgg, cctgctctaaattacc, ttctccgccctcattc,
tcttacttccaaccct, acgcctttgtgtgaaa, tcggtagttttttttg, actcataagcatcata,
gcgatttgcctgtttg, cttgcgtggcacacag, ctctagcacatattct, cgtgttactctgtgag,
aacaacacgtaatttt, ttgtgtatgatgcctt, tcgggccggtgcaggc, ggtgaaattcgctgta,
ttcgtcaccccccaac, ctaaccttgtctcctg, atggacagggagtgaa, atatactcgaatcaat,
tatggcaatacaccta, acggtgattttaacta, ttttgcctcctccgtc, ttttgtgtctacttag,
gaattagcttttacca, ccattacctgaattgt, actgaccgtattttta, cgctcactttcttccg,
tggcactctcttggcc, actaacaactgtattg, cttgtttgacataccc, tcgctaactaggccgc,
gcggccgtctcaggct, caattacaaaaaaacg, cctcgtccccctatct, gctaaatgcaagaggt,
tctggattagttatac, ttccttaagaattgcc, cacccctaaaaaagct, gaacccctgaatgaac,
gggctgcaacttgttc, acataagaaaagcgaa, cgggcatacaaatctt, cgagtatctttttttg,
tgagtagaccctctgc, ggacatatgccctgag, ctatgaggtccacatg, aacggtttcccctctc,
ggagtctggaggcgcc, cgtgttgggcggctcc, gttcctaacctagagg, atgtccattatgtcag,
ttaggggggaacctaa, aggcggtccgcgcgag, tcttaggagttacccc, tgcgtacgtgtgtgaa,
tcttaggaccatagtt, gaagaacattatcttg, cccggtctccccctta, agggggatcttgatac,
tacattaatggttata, gtgtatcccgggggat, cactttgggtcactgg, gggcatgtgtaagtgt,
tcttattaccatactc, tccacgctggggggca, ctgtgaagcccccccc, gttggaccaagacctg,
ggagcataagacacgt, cacttcccgtctgaga, ttttgcggattatgac, tctatactgctggaac,
ctattattccctggag, aaaattagccccccct, gcacgggggagcgcgt, ttcgtatactgagtcc,
gcacctgctcgtaaaa, tgggctactacaacta, gcaatggattttatag, tacttgggagcgcagg,
ggacataccctatata, tggtgctccaagggct, agactgagctctcgtc, acgcgttcattaggca,
atcccccctttgaaga, tgttattcctgatatg, gactttaaaagatctg, tttcttccgtgactct,
cctccgtgcacacgcc, gctttttgtgggaaac, aacccgactctactca, agcaacgaattatgtg,
ggtacataggtattag, ttctatttacccctct, ctgaggggttgacccg, tcgttgcctttctgaa,
ctttcgaggttttgtg, gatggacctaccccct, aaatccacaagatacg, tccattacatgtgtgc,
tattgcctgcgcagga, tcacgaggacagtaga, gacttttttagctatg, aacgcggttaaacccc,
aggccatttttagcag, cggcgctgagcgccga, ctgattcctttttacg, aggggaaatacccatt,
tacctctctcatccgt, tgcccccccccttcac, agacccggataggggg, gaatggatctgtcttt,
actgtgcacttcgtca, accacaagggttcttg, tgtgacacttagcagt, aaaaggcgaactacga,
aaactctgtggtcacg, ttgaatttagcttggc, tgcaacttttcaagtg, aaatgtcaacaggccc,
ttccgcggccgtctca, gctatagtgatggcac, aaaatacggtcattat, gctgaagatgccttag,
cggtggctgagatctt, ggggagtacttagcag, ctacatagaacagttt, gttctcatagtaacta,
gcttctgccctgctga, ggatctagtctcactg, acagcgaagagaatat, gaggactaagcaatta,
acccagttagacccca, ttcccataggtctata, tattaacgaatgcatt, gaaagagcttgacggt,
ccgcccattgggagcg, caaagactccacttgc, ccatgtcacccccccg, cgttagacataagttt,
tccggaatggtgagga, ttgaaggacggataga, agttggcaaaacatcc, tttatctagtctgacc,
gcggaatggaataaaa, ggctcccgtgggtgcc, gggggggtaggaacca, cgccactcaggggccg,
gcgccgcgggacacat, cttgcagtgatgcgag, ccaagggaggtgctgt, acaggttcccccccaa,
tgtaacactgctgaac, gtgtagaacaggccag, aaccttccctagctca, atacccaacggactgt,
gttgtgttacaactca, cggtatgtactcttaa, tatgctggtgaaaagg, atagacattccggacg,
cctccttcgctggcat, tctttgttaaccaggc, ctggcagcccttgatc, gaacatagcagaaatt,
atcacgaacagtatag, cctactatgcttcaca, aatgtgacccccccac, aatgccggcagtgacc,
acaagcacctgatgtg, ctcagcgccagcctca, ctgagggtgggtgaaa, aaggccatggttcaga,
tttcaccggaaactgt, acgctgaggaggggcc, tgttttttttcgtcct, tgcccgggccccatgg,
tccatcacttagtaaa, gattggatgttcccac, ttccacgagttttttt, gtggcggggatcttca,
actatgctccatcccg, actgcagagcctctaa, agggacttatgagtga, tctcatgattctagtt,
ccagcgaatttaaatt, ctgtactgtgacttaa, ttttcgtataaattat, atcatttatcccaccc,
gcatatagctttttac, aaaagacatttcgtgt, agtagacactgcaaat, tggaaggatatggctt,
tctctcctatctgaac, agtaaacctggtgagg, aaaagcagcaattacc, gaagcgtggtgtcttg,
tgtcttgtatgagact, gggattttttttacac, atacatgggggggata, actaacgcattgttcg,
gggccgggggggctaa, ggtcgggggccgtgga, atacaaggggccagcc, tctagctcctaatgtt,
ccctgtttaatgatcc, cgtgctctcttaaaca, taaaccaacacctggt, ggcgtaggcccccccg,
ggtgtaatttagtaca, aagtgccagctggtgg, tacaatactctctatc, tgcttcgctgctgtgt,
aggtgctttagaaggg, taacatctaacaattc, catcacgtctgcacaa, acatccgccgctgttg,
tgcacctagtccagca, ccacccctggagttag, ggatcagaaattccta, aacattagaacaacct,
ataccgagtgtcgatt, cttctaaaagcgttcc, gataaattcgattaaa, accaaggggtcatatg,
taaacagccaccttgt, agtgggtagaaaatag, atgcattgcattcacc, tgcttgggggagctgg,
tgatcaatggtgagag, aactctctttattacg, gggggcctttggcccg, ttacaatctgacgttt,
acatgtatgagaaccc, ctcgtgatagtgcctc, ttgaccgcgttagcca, actcggggttttttta,
aacttgaaacgtgaaa, cctcaatggggagatt, ccaaagcaggtcaatg, tttaacctttatgacc,
gttaaactctatctca, ctcctaaaagtcagac, gcggtgcgatctagac, aaaagaatcctcctat,
gcaaaggctcggtgag, ataatgggcgtgagct, ggtttacgcatcccct, acattgttccctgcac,
aggttagagaggacta, atatgttggatatacc, ccctgggcgggcacat, atggatcattactcaa,
cagacgacagcccaag, acgttttgtccggaga, ccggcgcgggctaagc, cggttagaccccgtct,
agttatgtcatagatg, aaccccacatgcttgg, tgtcgacaccccaggt, tgaccaagttagcccc,
gggcatatcctatctc, tatactgccagacgta, ttacccccccaacttt, cctggtgttccgcggc,
agtagaccccccccat, aacaacacgtgcttga, actggcctttagttca, atggcctgtaacctct,
agagaaccccccccca, gtccagtggggggcaa, ccaattagacttagat, aggggtctgggggggg,
agctgtcagtccgtgc, tattgtgaccctgaca, attatgtcgatactat, attttttaggggggga,
agccactgaaccttct, cggttttcgggtttgg, cagttccaagtgtaaa, tagtggttcacccagc,
ggggagattgatatga, atgttacctagaactt, tgtcaatgatttctgc, gggatgacgggcgtgg,
atctgatgaactggat, catgtatacttttcgg, ttaatcgtgttctgga, gtcattgtttttttac,
aaacgggtgctcctca, gtaggtactttgaggt, tatatgggggggtctc, ataggatggctgagta,
aatttcgtattaacaa, gggcccgttactgagg, cagcatgaacttagct, tgtagctgaatactgg,
gctcctgcctcggaca, cccttcaacctttatg, gcatgtctcctattgg, acaccgccacccggag,
acggaatctcgctgct, gtgagcctccccccct, tgccgagcctaagctg, gtcccagaaacgtata,
ggaggcttaggaatga, ggccccccttactata, atgacgaggtcctgct, cggctccccacgtctt,
gaagagcttggcccgg, cctacggtatcaccag, tctgtcactccttacc, gcctgtaaacccagac,
gccagtttttttatca, cattgctcctgtgact, ttcaatccttatagaa, ggatcgaagccggacc,
aatataggagattcaa, cccgaacaatgaaaac, gggcaaggctcggaag, gctgacggaagaaaag,
gcattgtaggtgtaag, cctcgcgatacatctc, cggtaaccgattagaa, cttgtcccgaactccc,
aatccttgaagctccc, ggaataagtgcaattt, ggtatgtctacctact, atggtgctcttagcct,
agagggctaaaategg, tcaaaaaaaagctatc, taatgatggccccgtg, gttgctgtagaaaacc,
tagtggtaactgacat, ctccgataccaaaacc, ggcggcggagctaggc, agaacagactctatgg,
gctgtggttaggatgc, agtgggggggggaatt, gttttaaagcgtgtac, taattgtagatctgta,
acactctagatactat, agtgtagtctcatcac, cacctgctggataggc, aaatccccctagcaag,
aagattccattctgca, gcccacgtgatggaaa, taggatgccctggatg, cgagtgtggggcccct,
ttgcagaaaagtaggt, gatggcgtggactctg, gatgattaaacaccag, atgcagcggatccttt,
cccgacagggcgggga, tgtcagcgattggctt, tatacgaattgttaaa, tggatgctttccaagg,
cctttttggccacact, ccttatgaactctaga, ctgtctcgaaacacaa, tgaatagtgctgcctt,
atctgctgagagacct, tcaaaacactcagtgg, atctccaacgtttctg, gccatgggccctgttc,
tccactcggcagtgct, taaggactctcaagtc, aatttttggagcgcta, tatctcgaatggtaac,
ccagccctcgatttgg, actcataaaatccaac, gcctctgatggctatg, gagaagtaccatttgc,
aacagatgatgtacag, tccttccttagggacc, agaaagcctcacctta, aatcgactttttacaa,
ataagatcacttactg, ccaagttagcccctga, cacgtcccataccccc, acctogtttataaaaa,
tgggccgggggggcta, cgggatgaggccatgt, caaccatccggtgatg, cgggatcttggggctg,
gacctgatagcatcct, gctgtaaataggagaa, ggcctaagtaatcttt, cctccccatagtgacc,
tacctatataacccat, ttaacgtgccggtttg, ctataactccgctttc, attaacttatcctttg,
gtaaggcttaatagta, gcttaatccaaccctt, ctcggttaatacggca, cggcctgttggattaa,
ccggttagagggaaaa, tgtggcgatttttttg, gcagggttccgtctta, gctggcatccgtgtca,
gtctagggacaaaaaa, gtccggaacacagggg, cgctccagctctcatt, gctgtgtctgcttaga,
ttgtgggtatctatcc, tgaagcacttgtgagt, cctgcccctgtcatcg, ttctgattaaatcctc,
gaatccttgaagctcc, aaataacgaggaaaag, ctctatagtccacgag, ccctgagagagggttt,
cctggtgtggttgact, tataaggtacagtttt, taatcgaagtagacag, ggtgttccgcggccgt,
tagtaccttactgctg, caggttacagggaggt, gcccattttacccaag, tggacatctgatgatg,
gtccatgaatgtatac, ggtccgtctcgccttg, atccatcgtacagtgg, acttgctctttgcaag,
atacgaagttcagagc, aactacagctctacta, tattgcttaatgacgt, caaggcctagtgcggt,
ggggggcattcatcat, tccgtcttttttttga, aagggagtggaaacgc, ggttccccagacggac,
tggtggggtaactgct, gttaccctgatctttt, cgtccccctatctggg, gcgttttttttctggc,
tcaatttttggagcgc, ataaacagcacccttg, cctcgattttaaaacc, gcttagaataaaaact,
agagggttgattctgt, gcctatacagagctga, ctagtcgggtaaaaaa, cttctgtgcttgccgg,
ccttatcatgctcaat, atttacacatgccccc, ataaaccagacgaaaa, tattggtcgtatatat,
atgtagcaaggctata, tgatctaggtgatgta, gtaaaaaaaagagtgc, taaagcccctatctca,
ctagccaagatctcac, cgggaccccagatata, gtaggacctcccctgt, attttctgtgaaacga,
cctatagtaatgttta, cttaccatcacgattc, ggtgttgttgcttttc, tgtcaggaaaacgaga,
ccctagagtgggatac, cggtgattttaactat, tgttaagggggggaga, tagtttgtatgaccca,
ctgcatatcttcctag, ctctatcttgcacaat, tcttgcaggaagagcg, cacttctctatcagcg,
aacgctcctctgtcct, gacttcactccttaga, ggtgtgtggaactaat, caatgcggtaaaacag,
cataattgtataactg, cacatagggcttcatc, accttgtccacctacc, ggcagtgactattgtt,
tgggcatatcctatct, ttcctagctatttggt, acatttcccgaaaaaa, ccatagggaaggctct,
ttacgttctgacagaa, tattacccacttgctg, ttccgaatcttcacag, ttccccctggagctag,
tagcaaaaaacaagcg, cctctctgttaatgca, tgctaaaatttattcg, ccaccaagaccagata,
caggaaatttgcgcag, cgtgggaggtgccggt, agtgtcacaagaaagg, attcacaaggtacatt,
gttcccctccacagaa, aaacatagggaatgtg, tgttttttttaaggcg, ctggtctttatatgaa,
cggcatccttgaggcg, aacttaaacagttgtc, tttaggttcagacctt, ttgccctcctgctcgc,
aaagtggcgcaaggat, aggtgctaggaggact, tggcccctaagataag, ttaagaggcgtttcag,
tctgatgtgtataact, ctgtgttataccgtct, aattgcgtacatattt, tattctatgtaactta,
ttcaaaaagtcccagc, gggagggggggggtgt, agcgttcggcctgtga, caggtagagcttagag,
gtgtcccttcaagaag, ggcttccgtgtccctc, gccactccagaggctt, tettccccccccatct,
aagttaggacatcagg, tagcagaaatgagact, agtatgaattaagcaa, acggggattgtcacaa,
agtactcctttgtgga, gtacataaaggagctg, gtggcccgaggcagat, ctgtactggctagcag,
aaggccgcggggtact, tgcccccccccatgga, catgggttctcccagt, acagacttcccattgt,
cttggcccggagagga, acagggaatgagctga, acctgtaccctaagac, aaacgatcttacattt,
taggtacctacaatat, ggtgttcgggtcccgg, agacttgaaggtagtt, gcctatagatatatgt,
ggacggcactcacacc, cgagaaaatctggtat, atcttccacctgactg, gatatagaatatggag,
aaggcataccttgatt, acagtctgtataactc, accaatttagccagga, tgcttaggatgcaggg,
gacccctctgagccaa, ttttagagtgagaata, aaatcgaaaccgtctg, ggcaagggtttagggg,
gccaaaattgacactt, agctacagcgttttgt, tatgactcttgcagcc, gtcacttttttggatc,
gcttcacaactgctag, agtttatggtgtttca, cttacttatctcatcc, tgctttggcccaccat,
ttacgatgttggagca, atcgccacagctaagg, gtttcattagattata, gttacaaggatattcc,
cgctgaggggtgcgga, actttttttagccatc, agcagcgtctttgcta, cctctgccagtaatct,
ggatggatcattaggt, ctcacacttctaaacc, cgatgaggattaccca, aaaatataaccgaggt,
cgcaaagtgtcaggta, cgccccggtctccccc, cagactctagtttaga, aacttatcggaggaca,
ggcctgttaaacaggc, tccattaggaggccta, tgccctataagggtga, cacttgagcccgatag,
cacactcactgcatat, tgttgttgccccatgt, gtgatcccccccctcg, ccactaataggatctc,
ccatgttctgaacaca, ctgaggtccacatgaa, acaacgataattaaat, agacgccttgcgttcg,
tcgggggccgtggagg, agaaggtcttgtgaac, accacagtacgtggtt, ggccagtcccagtctt,
gatgacgggcatttta, ctactcctgtaggtct, cttttttcactacccc, ggcgtcagttataagc,
acggattctcagttgc, atctctttgcagagac, gtggtcctccttagtc, ctcgcaaaaaaaatcc,
gggcatcaagctccga, gcacttcccccaacgg, tttttttccgtacttg, cttgtccagccgccgc,
gaagaggcataagttt, ggatttagacataaag, aaagggagccctttgt, ggtccgaacttcctgc,
ccatcttaggtaccag, tacttgctaactggtg, gcggtccttccggggg, cacactatgctaatat,
atagatgattaaacac, ctcacaattgttatat, tttccactataacgct, tgcttgccccatctgc,
gagccccatgtattac, tgcgagatcttgcata, atagacaagttatatg, ctgcgggtttccattg,
ctcttcagccctagct, ggtgtgccgagcctct, atttagattgagccct, tacctagtcactttga,
gtgtgcgtgcgccgcg, gccgggaagagtcgct, agcgaattctttattt, agagatcacatctaag,
gaatagaattagacct, aaggtgaataccatgt, cggcgctgaggggtgc, gcgggtccagcgccta,
caggttgggtgatagg, ccaaaaaacggggcta, ttccctcccctcatta, caccatcctctatagt,
tgactaaattgaacat, gtgcagggtgtaagct, tccgactccggggcaa, gtttattggaaattag,
aaccttaaggtgccag, tgttcagagactatga, acagttgctcaagtgc, gggtttaggggcctag,
tagcatggctcaggtg, ttggtggtaggactca, cgtcgggacgtcccct, tcgtgcggagccgaag,
gttagtcctgtcaaca, gaaacatacgtttgga, atgatgaaaaggggca, gtttgccctttttagg,
agagtcaaccccccac, aactttttttgctgcc, ttataacacagaccca, gtacagatatcacaga,
aatatgtccatacctt, cacatgacgggaagac, gatacttgactcagaa, tcgctcaaataagatt,
acagccggcgctgagc, tgccccaacattagaa, aagatgtgctgtaaac, tacccggataatagtt,
cgcgcaccactgctcc, taatacaacgcgcccc, gacctccggaaactta, tttattatagcctcgc,
ctgcatagcccccccc, gtctaaggataatttg, aatccccccccaggcc, tttggtaatttagccc,
accatgctcccactca, cgtacagtgggctgtc, gggcgtaggccccccc, cttatggcagcatgga,
cgcagctcctctcttc, gcaactcatgatggat, ctgaggggataagacc, aaacctgggggggtca,
atactgacacagtttt, gaggcaggctgaatac, ttgggagctccacctg, atctccatcctaactg,
aatgccaagcattaac, gcttgctccaccagac, attatgatgctaggct, aatgccaaagcgcctt,
cgcgcagtattctggt, gagtagtatattatag, gtgttataccgtctct, gaaattacctacaagc,
gcgcgcaccactgctc, ggtaggccaagcagga, ttaaaggagccaaaca, aacctcaacttctctt,
agcctatattacagcc, cgggagggttagacag, gcgttggtgcatgttg, gcataaggctagcttt,
ccagtaactagtgaat, ccttccccccccaaag, gtatgccctatgaaag, caattgccatttgcac,
ccccatgtagccctca, aggtccaaacaggcat, atgttactcttccgta, agcagatttctcggaa,
ttggggggggaggcaa, ataaaaccatccggtg, caggctaacatggtta, cacctgctcgtaaaag,
aatacctatccaccca, tcagaagttacatcct, gctcaaaaaaaaacta, gaggcggcgcggcctt,
ccagccgtgccttccg, acaggcgcacaatacg, ttgagattagacatac, actgtaacacttatcg,
gtctcgtgggtcatct, actccagcctgtggcg, gtccacccgcccctac, tggtggatagatgtga,
ttagttgcttcacctt, gcactatgtcatttac, gctcccacttattctg, tggaagaagaacgact,
gttagcttacatgaag, ccccaaagtactgcac, cagacgccctgtccgg, gctttaagtgttcgtt,
ccgaggcagatctatg, tgatgatgggagtgag, atatgttaaacgaggg, ctccagcttagggaaa,
agggggggctgctttt, gtctcgcaaagtgtca, tgctgcgttgcgcact, ctccgcacccgccgtg,
atgcataatgtgaagg, aaaacgtctagataaa, ctcatcctcttagcat, tttagatccacattgg,
gatgcagctcaaggtt, ttccttatttgcatca, tgctgactcctctatg, ggtcttgatgaagatg,
cggaaacgcggggtgg, cctgccagctatctaa, gcggggtggacttcgc, aggtgggtatgcctca,
atatgcgcaacacatc, cgcgggcgctttccag, attaaggatggagagc, ggtccaccccccttag,
ccagtgtaggcgacaa, agcttccactgtccgc, gacgggttttttttgg, ccctgttcttgataaa,
aagctaggtcaaatga, acttgatatcaacaag, ggagataaaggcattc, caatttatctatgtat,
actcctgttttagtat, tataccatcgttattt, gcaacgccgaagacag, tttgtccggagaatac,
cagcctttttttaggc, gattccaactactcaa, ccttccggatgggcat, acagcctttcaggtag,
agatcctccattagag, acgataattaaatata, acgcattgttcggatg, tgtcaagtgctgattg,
tgcctgcgcaggacta, atggacctaccccctc, gcctggccctatggcc, ttctatgttttcttcg,
cacgtgatggcaggac, agcaccgtgcatcgta, gagcaggccattctag, gcgctgtattaacttt,
gcttatgtaagcttga, tccatctgccccccca, gttaaaggagttgggt, caacggtttcccctct,
tcaaaaatgatacgct, tattcaaaaagtcaac, gaatgtgggatgacgg, tacacccgctgaagga,
ccctgaggggacagtc, tcgatgagaggcctgg, tccccggaccccccac, cttactgattgctagc,
ctaaccggggcccata, ctttatctagtggtca, acctttgggatactgc, aagtcacagcgctttg,
tctaggcaagacttga, aaggtaacacctgtgc, cagggttccgtcttaa, acttttttggattgat,
ttctacatgtgcaggc, tagaagggtatggggc, atactttggcccagcc, tctaggcacctcagaa,
tctgatgacttttgtc, atccaggtgctggata, agaggagggggggctc, gtactattatcaccaa,
gtcagcgtagggctgt, tttggtagcattaggg, gtcctgacccccccct, atgcttagaatgatat,
atctaccctttgaagt, caggtgttttcgaaat, ctacttcttaacatgg, cacttgagtaacctcc,
ggcatagtaaatcaca, cctgcagtatggggcc, ttaggacaggcatttc, ttccccgcagtgccca,
cagccccacttggatc, tgacggtagttttttg, cgtgttgaattaaatg, cactgcttgttctatc,
ctttcctatagtgtat, gaggccgtagggtcat, ctgatgccgagcctaa, caaaagatataatggt,
ggtgcctaaaaaaagg, cgccgggcgcattgcg, attttagaggccaatg, gctcgtaaaagtcatg,
acagcctggcaatgcg, acttgaatgtatcagg, ttggcctcagtttcgt, ttcataaatgaaggcc,
catagtgctttttatg, aacaccagtctttaat, atagaccttccggatg, gtaaaaaaaggcacac,
ttattaggcacatcca, tgagccaattagcttg, tcagatgatgccggag, tctgccaagatcccgg,
gtcataacctaattgg, gtcaggaaatattagt, ctttgtctcgtgtttc, atccaaccgccttaga,
ggatggtggctaatag, agtctgggtatggggg, agcatttttccccccg, ctcagactgtttatta,
agacctgggatctctg, gcactctctgagacta, gcggtccgcgcgagag, tggaaatgagcgtagt,
gtatacagcacccctc, gctcaaaacactcagt, ggagagcctctcctaa, ttgttatacacaaggc,
tactatggggcaaagc, ggtattttttgccagt, gctcctatgagctcat, atgcggataccttaga,
catttcaggtcatgac, cgaggcaggctgaata, ccaaggagagtatctt, tcatggggggtatact,
tgatattctgttgggt, cttagaggtgctgatt, atcttcaatattaggt, ggtgagcccggggggg,
tgctcctaaaggatta, tccgtgttgtgcagct, acatgctggtttatgc, cagggaacattaatta,
taattggctgatggct, catagtagactgtgcg, ctaacctaagaggtat, atggcagaataggcag,
tatacaatgccatctc, aggcgcccgctgcaat, tccgccactagatggc, atgcacttatactgga,
atcatactggccatca, cttgcccatcgggaag, tccataagaccttaaa, atgagcaacgccgaag,
ggatttatcttacggg, ttcgtttgtacttatg, tgagttagaacacgcg, gctggtggtcgcatga,
gegaccccgccctgca, tgcataagacaaattg, tggatgtgttacccgc, caaatagcactcgtgt,
gcttggttaggtgtgt, cccggtgttcgggtcc, gcgtgtacctcccaca, acctactcagcttttc,
accttgctaattattg, gtgtcacagtcaggca, cacgccgtagtcggcg, aaatgatatgtctgcc,
gcggcgcggccttgcg, ccacgctcccagatga, ctgaggaaggcagccg, ctgtttttaggggggg,
agataccgagtgtcga, attagtttttttttcg, ccgagggcggccttgc, ttttaaagttcgagag,
gttccccccatcaact, aaaccactatatcagc, ccgctcttcgaggtgc, ctctgtaccttagggc,
attcttacgggcttag, ggggcctttggcccgg, gcctagctgttaaaca, atactcaggcatggcg,
gcctgtctttcatgag, gatagtcaaaaaaaat, gcggagcttactgaga, catggtttaagccatt,
gtacccttggaatgca, gtcactgatgtggtaa, ttaaggtctcccaggc, atagcaaaaaaaacgg,
agtaagcttaatgttg, ttgtgcctcagacctc, cataccacagtaaaac, gtgggtatttaacact,
tttggcatcctgtgtg, ccttgtcatcaggcga, gttaaacgaatgcagg, ttaccaagatttcgga,
acgtgtttacctgctt, gtagatccgagttttc, tgccacaaattagtgc, aatcaggggcacagtc,
ccattgctcaactaaa, caaatcttaaagtgtc, ttagagccattccttc, actgccaaaacttagg,
catggacatacatagt, cgattgtaggaactat, aaaatcttagtacgaa, cttggggcgcaacatt,
ttttaggggggttgtt, tgaattgacaatttac, ccaaagcataagaggt, tgacttgaaagtatgg,
acactaaacategaaa, caggcggatcgcctta, atcccccctaaatgta, catcttaagtcccagg,
gtctatgaaaactcat, caattgcagtactctt, gctgatattatcttct, gctggtgatttacatt,
taaggaagtcgtatta, attactggaattgata, ttaggtaagagtgcag, tgcatgggctttcccc,
aaggagatcctgctcg, gaagtcttatccatgt, aaagtgcctttttccg, gatgcctcaactgatt,
ggaggccacaccgcca, gtcagttgtggtgaac, ctcttggagggctgta, tataataggcctattt,
agtatgtgggttgttt, gggatcattttttttg, tgtgaggctgttcctg, tgaaggggcttgtcta,
gcattagcgaacacac, acccggggttcgtctt, taaacgggtattttta, agtaaaggcttgtgtc,
ccgaaaaaaagcataa, cttcttaattgacact, aatttacagatcaggg, caacggctggggggag,
ttagcacaggtatcca, tctcgaagctctcacg, caaggccatgatagcc, cactgtgatcttatct,
gcggactgcagtgtcg, ctgaatctgcgttata, agtgatctatgaaacc, ggcctgccgcccattg,
aagaacggacagaacc, cacggtcacctctgtc, gcagcctagctgcggc, ccaagtgggggggggc,
agtaaactaccgtaag, catttgagcccaaaca, ctttagcggtgaatgc, acattttggcgagtta,
ctggtatagtagtaga, tactctgtcttgatgt, gaatccgatgttctag, cggtccgcgcgagagg,
ctaagatctagacact, aactattagatgctca, ggggtggagaatatgc, caggaaatcgaaaccg,
gactccgtcccccaag, attcctaagtcccgga, caacctggttaaacac, cgtacataaatccaaa,
agttgctcaagtgcta, ccatattgtcaacatg, ttacgctgagaaaagt, acaggctctcatccat,
caggacgctcccatct, agcccttaattgtttc, gagcgaggcgctcgag, atagtccacgagaaca,
ctctgtaatattgatt, ggcggggaaaaatgtt, catctcagaacgtagg, tgagttcgatgtttgg,
aagaacgaggaaaagc, gcaattggcttcccaa, taattgatagctatac, tacctgatgaacttcc,
taggcttatacaaaaa, gaggagcataggctga, aaaccctagcttcctc, ataacaactatgtgga,
acgccagggtggtagt, cggtctccccaactta, cacaatatcggattta, acatggtcagggtgtc,
taccgtctgcccccaa, tcccagaaggtgctct, ttacgccaagtgccct, ttcggggggaggtttg,
tcatgctgcaaagtag, tgcgcctcccgctatg, ccttctaacagtctcc, ctttgagccaggtaag,
cgctctatttctcgct, agagggaacgcaggct, gagcatattactcagc, acataggcttatactg,
gcctgacccaggcatt, ccagtttgggccggat, atataccgtttttatg, aggaaggttatggtag,
tgtgttgaatctcact, tgtgagggggggttgt, atcgtccatggaacta, ctactgaagagggtga,
tgcagggataatccat, acctagttttggggaa, ccaagctatgataatg, aagcttggtctggccg,
tgtattatctcctacc, taagaccttttgtaga, gacgccactccaatcc, ctgcgcgcggggctgt,
ggaggacaataacctc, cttaaaaatcttcgaa, ggagcctttgcaagtg, ttttttccgtacttgt,
ttaactgttccctcca, gaagatggcgccggga, agtaatgattaggtga, cctcagtttactgttc,
ggaagggtgagctaat, tgatcagagaccagac, taggagtgaatgccct, ggaaaaggttagacct,
tcaggttaccactggg, gctctcttagtaccca, ccgaactttttttagc, aagccggggggtagct,
atgcggtaaaacagaa, tgacctatgtaagatg, ccgagcactttgcgtg, gtagagcttgcctgta,
ctctcctcactcggcg, taacgtgccggtttgt, gtagttctgacaaatc, tgtgaggagggggggg,
tgattgcccaccttgt, cttgcttctgcttaac, gctgaacttattgcct, caatgcaggctatgcc,
cgcggggactcggcgc, agagttagcccaggga, ggcggatatttgcatt, gtaaatgtttcctgcc,
ttctcttaattgcgat, aagttattagggggtg, ttatgggggtgagtgt, acattaagcggcataa,
agtggtttactgttta, agtcatactcccctat, cttactgtgtttgcta, ctgttcctcgaaaact,
cacttcccccaacggt, gcgctgccgcccccta, ctggaccccttatatt, tttcctgtgaatatgg,
cgatctgccctcgccc, ctgaggatgctgaccc, tcctttgcctgcaatc, tctgtcccggggacca,
gggtgattcaactgta, tacattactcttttcc, ggaggatttatcttac, agttcgaaatggaatc,
ggcgagaccacacttc, gagagaggactcccga, cttattaatcgattaa, cccaatatgtgctcct,
gggagcttatttgtac, agagagggagcgcgcg, agggcgaagtcacctt, aggtggggggggacat,
atagcatgttctgaac, acgcgccccccccact, tttactaactttagtt, agagaaggctcaatta,
cgggcatctatgtcag, acctccctctgcggaa, attaggtcggggatgg, atctacactttccctg,
ccggaatccccaaggt, attcaatgatcaagct, ggggcatcaagctccg, aacccagtagcagttt,
tttatgatgagggtac, ataacagacctctaca, tcagagccttagttgc, atcgctttttttgcat,
cgtgcggccgcccaca, attgtataacctgata, aaagcagtgctggtat, tcgggtttggtttttt,
aagagttgttgagaac, ccacaggggttttttt, agtgacggctttggca, gatgtgacgagaggtg,
gattaaagttcttcag, aagctattatgggttc, tggtatctcgaatggt, acctcgaacagccaag,
atctatgagagttaga, gatcaggcttttttta, agcagttagttaaaca, gtcagcgtgtggctta,
tgtttttttttccgaa, tgccggagcagaacta, cactggggagcaataa, atcactgaacttgatc,
cccttccaccctgcta, tgcctcagtcgtccaa, tgaggggggtatatcc, ttacatatgtcttcta,
gtaatatgatttgttc, catgttttttttgcca, ttatgctggcggaaag, cggcttagcgccgcgc,
gtttgagtcccatgct, gtgcggatcagcacaa, accaatcagccggatg, gatatagtgaagtacc,
agaagctatgtacagc, tgaaacatggggcggg, atgggggggaagatgg, aatagtttagtaccac,
tttacataggtaggtt, aattgatggctgacta, tcatccgatatgctct, cccttagttgtgtatt,
ggcgctggccagctga, tgtttttttaaagcgc, tacaaacctatatttc, tggataatgggggggg,
gttcagtctcccttcc, gtaaaggaaagcgatt, catgcctgaatctgcg, gaaacgtaatcatcta,
gagggttgcaactgtt, taccctgtttttctat, actaattcaaactggt, ttgcaagacaccatga,
atgtttctatggagtg, ttggcttggcttatta, taagtattagtacgtt, gcacagtgccaaaccc,
aagagtaacgttgaat, gacaagtctgagggag, tggagcatacgaagtt, acaaagacccatgtcc,
gctggtgattatcctt, gactactgggcctgcc, gacatattgttatcat, tatactgctggaacgg,
gaattcttggatagtc, tggactctcccccggg, cttcgacacggcttct, tttggactcagcccga,
tgacttggctccttgt, gtatttgtttaacctt, gtgtgcgtgaggagaa, tcctttctgttcgtaa,
ggtgtttactactcct, gtaaaactccggttca, ttttctcagtagaggc, tgtaacacattgcatg,
ggcttaggccacttgt, agctggattttttagt, tcttcctgcacgggca, cactaatgcagtacat,
gaatgccctcaactca, cgctgagcgccgatct, aaacggggattgtcac, ttatggcgtctgcttc,
gtcccctttttttggg, ctcttaagccacggcc, catatcagcataatga, cccacgtacctttttt,
tatgggcgtaggcccc, attctaataacacgta, tttttacggctcatct, gttgtcttgctctgta,
gcggtgggggggatct, cctaaaccccatatag, agttaaacacagagag, gtcttcgtaattactt,
ccgatttgcttaagat, atgccaaacataatca, gcggtgaggatgcaaa, ctctaccacctgagtc,
acactcattgttatgt, ctagtcctttgtccct, gcccggcacttcccgt, tgctctttattgaagg,
ttcccttagggacccc, gatcctagctgggcta, atgttgtccacaactg, tcaccccccaagtaga,
gagggtccaatttcta, gaatctgcgttataca, aatcccctctactegg, cagtagctctgtctta,
aatgtccacttgtatg, tacaccttgtgttttg, tagacttgctatatca, acacggttagaccccg,
gtagcccagcagtccg, atgcaaaaaaaggacc, agttacagtgtaagca, gactaggtgtgactat,
ttttgcatgagccaaa, catgctgtcgctttcc, tgatggccccgtgtga, gaattaatgttacgtt,
aacgtgtggcctgact, atctattaaacacgct, tgatggggttggcttt, ggcaaaaaatgacagc,
tgagcaggccattcta, caatcaaggtggagca, aggacgggctgggcat, aagcatcataagttca,
aatgcctcgatcagtc, tactcaaaaaaaacat, ggccaaccacccagta, tctgggagaccgagcg,
cagattaagctagaaa, tggggcggggatgttc, acctgaaagttcggaa, tccgtaaagtcaggat,
cccggaaggcttaggc, ctagtcacagactctt, gttttatgtagactta, agtcaggcactaactt,
aactgcaggtgataca, gactcccctgcagtat, agtgcctttttccggc, gggcggcggagctagg,
ttctgcctgtgcggtg, ctttccccatgtagcc, atatgcttactacacc, ctgatctccatggagg,
agttgcttgccatgtg, tccttggacgtgttga, cgattcttgagcaggc, tatcaaactccacctg,
cacttgtgctgagcag, tgggtagaatagacaa, ttttaactccgtctcc, accgcgtcaggctgtt,
agcactttttttagga, catcctgtttcgaggg, gggcttgtagtaacca, cttaacgataaatata,
atccaccgaaaaaaaa, ggggggggaggctttt, acttagaatgtggttt, tggcgctggcagggtt,
ttgttgcctatttacc, ctgtatgacaggatct, acttagtgcttaagag, agagcgctggtaaggg,
ttgggggggggagatt, cgaactttttttagcc, ctaccccaactaatcc, tcaagcattggaaaac,
gcatcttgcgcgagcc, caacctttggtaagcc, gtttagtgaggcaaac, cgatgaagaaacttag,
agtatcccccccccga, gacggagttgggtgaa, ctgccccccccaaatc, gggggggcattatggt,
tgtgtactgatgaaca, ttgccgattttttttg, tgttagctaagttgca, gagggaggtccccata,
gatcttagagaccctg, caggcagacgtgcgtc, ctgcagattgggctga, tatgactccccgcatt,
cacaagctgtctgtat, attcgacattttttta, ctgtctgactggaagc, cctgaccaagttagcc,
tgctgcagtaagaata, ggcaccgtgcccctcc, cgctactctttttttc, ctaagttatacttagg,
ttgagcaataagtttg, tcaggacttaaaagct, tgcgccttccctgaca, cctgcaagagtaccat,
taggggggggagagta, tcaaacgaattaacct, tatgacccctatgcct, cccggactcacactgc,
tcctgtggatccctta, catgatggggggggag, cggataagacgctgag, gccaacagcgcagtag,
aagagacgaaaaaaag, aatgggtactactcca, cgaaaaaaaagtatca, acaactctacactgat,
aacactaggcatgcaa, gccgtgccatgtccta, ggcagtctgacaatta, ggtggttctacgctgt,
ctgtttgttcaattgg, gatttgtaaaaaaagc, ggatgtatacagtaat, tactgagagcggccgc,
agtgtacccccccagt, tgattggtgttcctta, cgacgatgatgaaaac, cattcccgaattcagt,
tcacagatgaatgtat, ttctccccacacatgt, acctactatgcttcac, ttgtcccttttttgac,
gggtgtctagacattg, atctcgtaaaaaaacc, agtgacctcggggggc, aacccattttttttac,
gatgcagtagcacgca, ggagcttaaagggcct, atgtgtccttttatcc, tgtgcggtctgggttt,
ctataaaaccttgcgt, gaagagaatagaagcg, tgcctttagacctcca, gtagtggttcacccag,
ggctaagccctcgctc, ttgaggggggggaaag, gtggaaagagacccct, ggatagggagattcgc,
gacgaagcagttattg, cttttttgtaccccga, gcgagttaatacacac, acataggtgttgttta,
agcttctatttcactc, tatacggtctctcttt, gttaaagggtgcatct, aagggtttaggggcct,
gattactagtaaacta, ctaggcatgcaaaggc, acatgcttgtaacact, gccacggagtaggggg,
cactaatacaccagtt, tctttgcatgtaccca, tgaacaaaacgtatat, cattccttttgactcg,
cttaagactggtgcag, ctacacattaggcttg, tttgattgctacactt, gttttatagcaggggg,
agtcgtccaagtagct, aggccagcccccccag, agctgattgggctagg, cccgaaaaaaggtaaa,
gtagaacgtagactac, cctcatgccaatgggc, gagaactatactggac, ggcgcgcaccactgct,
ttgccgcgcccgcgcc, aactcctgtagagttg, actaatgcacgcagaa, gcgggcagatgacctt,
tttaggggggggagga, tttaggggggggaggg, aggaccagactcactc, gacacgtctactctag,
gatagatgctaaaagc, ggaataagtggggtat, aatcacccccccaact, tgtaatgtagtaggca,
ctaaagtacttgtgca, gagatccttattgtgt, gtttaacagttagcaa, tgacttaacctttagt,
gccagtcctctttaat, ctcacggtgacttcac, tcttgttttgcactac, aatacatgggggggat,
ctcttatgagaaccat, cccgtatttggccatt, tagcagcccgagcaaa, ttgcttcgctgagcct,
gacctgtgccacccgt, gatatttttttggaat, aggtcaagctatagag, cacagaaagaacagtc,
ggagagagtgttttca, ccagacataaacactg, ctcgtggacaaaaaaa, gatatacagaacgtac,
gagtcgatttgttttg, ggtgtttatcatgaat, atgttctcactgaaag, acacaaaaaccctgta,
ttgcaacaggtgtttc, ttcgattgaagaaaac, caaagcttgcccctta, gggactacatgcgttc,
accctcgccttgagtg, cctcctcatgccaatg, ctaggataatgggcgt, ctccaacatccgtatt,
ccctatggcccggggc, ataaccccatccacaa, atatggcagcaactca, caggggcggcggagct,
agatctgccttctcga, gcagtacgttaaagta, cctgtggtatctggta, gaccccaaaatgtagc,
tagattaacgtagagg, ccttgcgaccgccctg, tgtctgttaacaatat, gctttccategttcct,
tggtccatagctgccg, aactcccaaccctact, cgtagcgaggcgcgcg, tgagtaagagccctcg,
tagctgacctcaattt, gtgcaaaatctgtcag, ccggggcccatagaat, cccacaaataacgccg,
tttcgatccactaaca, atttcgttaccttgtt, gcgtggaatcagctgg, ccacgatgaacgaaaa,
tcccctgagtagtcca, catgtcacgggactcc, aggaaccactagcacc, acggctggcccggcgg,
catctactgttcctcg, tatcacaccccaccct, agtcgaaaccctacta, tgagtagctatcacag,
tcttaaacgaatgctg, agattgatagattcac, ttttgtctcgccactg, tgcagcggatcctttc,
gcttaagacacccatt, gggagtaggttctgga, agcacttactgactca, tcagggtgcactgtgc,
tcccatgggggtgcct, ccctgctgtaccatta, gctaaattgaggtggg, tcttccgtacaatgta,
gggagggtccacgttt, cgctttttttggtatt, ctcaccgcatgacgag, gctacttattgggaga,
gtaaataatgtttcac, taatatgagtttgtca, ggcttgagcaataagt, ctctgctgttagcaaa,
ctgaaattagcaacct, acataactttactgac, ctgtaccttaaaagga, tgtaggattagagtcc,
taatgaagtttactct, tacttaatgccctttg, tggcaatgccattcac, ttctattgtaaggagt,
cgtagtttttttggcc, tcaagtggacgctacc, catcaaaaaaaagggc, atctaaccctgatgtt,
caccggttttcagggc, ccaccacgcctcttta, ctgccctcacatgcca, aacagtacatgatctc,
gataatacaacgcgcc, cacagattaagctaga, cccgtttttttattgt, gcccaggtttctatct,
aacagacctacataag, ttaggggggggtataa, tttgtatgtataatgg, aagcgtaaaaaaaatg,
cccttttctatgctcc, cagctagtctacttat, gagtgggggagtgcat, tcgcagttagggatcc,
tagaattattaggtac, cggcgccctcgtcccg, atcgaaaccgtctgaa, gaagagcgagggaaat,
atgcttagagctagga, ccgcaaggcccttggc, ctcaggcatgttgttg, aacttattacatagta,
aagcctgcacagctcg, gctctcatttttgatc, acctagctgttagtca, agctccttattgccgt,
cccgatgtcaagagat, cccgaatgaacccctg, cgtcagtgagttctcc, gggtagggggggggac,
tggggtacatgcactg, atcatgtaggattttc, ctgtgccgcgtcctct, aggcgggatctgtggt,
cttcttctcagttaag, atggttaccgcaaacc, ttaatcgctttaatat, ctgtaaggccttgcca,
tgtatattcccattgg, ataatagcgtacctca, gaatgtaacgctcatt, cggggtctcggcctgg,
agagtaggagcctccg, cagggatcgtctgggt, gagagggggggattgt, ctaccatggccttagt,
ggtgcagattagactc, tgggtgcaccccaatc, gggacatcactaccct, atgggggaatgccccc,
atcaaaagcataagtc, tctagttcaatgtagt, ctttcgatccactaac, catataggattggtga,
tccccaaattcccgct, ctgtgataaaacctct, gagtgcaatgaacctg, ctaatgttgcctgaga,
tcactggagcgttctt, accagtaccttttcct, tatgggaatggtgttt, gaagcattagaagaca,
cgccaagtgccctaca, tccctatgttgtggtg, gcaagggggggggtag, tgctgcttagaggaat,
attagccttcagtaag, caggtgcatgggtgat, tactaattccgttttt, ttttcacatagtgtgg,
catatttaacgtgtca, actatatgcgtaccat, gtgctttccggggcat, gctcatgcatggcggg,
aataataacacgacat, gccagacaagtctgag, cctgccagaagggtta, gatagtgcagccacca,
tocaaaaaaaacttgg, ctgaaacgagctgcca, tgagaagtaaccagca, cttgtaagcctcatct,
gtgggcggtccttccg, accaaacgtctgcctg, tctttcgatccactaa, cttaggatctacaggg,
ctggactttaactctt, atcactgtgtaccgtc, gttccacctctcttgg, cattttggcgagttat,
gagtaactatggtcta, ggcgttattggctcag, tcaagtgggtcactta, atacaggtggggggat,
gccgcgttgcggcgag, ccaagatttcggagtt, agcctggacgagtgac, accttgcctcgcctct,
gacttaacgccacatg, ctaaaatcggaaccac, cgtgtttacctgctta, acgtgattttggcttc,
cgaatacagttagaaa, gatactaaaaaaaacg, gacctcttaggagggg, ttagattaacctggag,
tatcttttttttgtcg, aaatttaatctatgcc, acctaagagataacaa, ctatgaggtaaagccc,
ttgtctgatgtgggtc, taacgatcattacata, atggtaaccccagttc, aaacgtaagtaatagt,
tacacattcccgaatt, gctaaaaaaaccactt, ccatgccccgtgtagt, ctagtctgaatgtctg,
ccgacagattatggcg, ggatgacgggcgtggg, ttaatgaatgcagcta, tgagaacacccgggtg,
catagagtccaaagct, tccacatgtctggata, ggtcttactataaagc, gatatgggttgagagt,
aatactcaacctaggg, gccctgccaaaaaaac, ttctcgcaccatttat, atcaaggatgttagga,
ggcgatgaggattacc, gtgggcagtgcagcac, cgctcaaataagattc, aggagcaggacatctc,
aaatgttgtccacaac, ggcgcttttttttcag, gaccattactgtaaag, gctagtcacagactct,
catgcgtttctactta, tgaggctcacctcttc, aacatggggggggcag, cattgctctactcttg,
ccggtcttgtcggtgg, agcttctaatgcccaa, tatggtaaaaaaagtc, gtctgactctgtgacc,
gcgctataactccgct, atataatcacacttat, ggggaggccacaccgc, ggacagggttagctct,
gagggtccaaagggag, aaaagcggaacagaga, ctgatagaacacgaag, ctcatgttagagttac,
tcaccctcctgcgggt, gttcagtcacaaaatt, ccgctaaaaaaaacgt, aacgtttgactgaatt,
ggactcagcttagagc, tgttttagcccaagct, tgaatatacctaacta, agaactattcccaaca,
ggtggttgtttcgtta, ggtcaccgttagcatc, cttgctggtggtcttc, gagagaggtacgtaag,
ttaagggttctctctt, gtccctgaactgtccc, actcaattgcgtacat, gactgttggacggggg,
cagtaaaagaggctca, acgccaagcaaatgtt, attaaggtgttgttgc, attattgccagtggcc,
ataggtagcctgagtt, gctcccccccccgctt, ggtatttatatttgag, aggtgtatctaatttt,
tctcgatgtcctgaac, taaaaggtttcgagta, tactacttaaccatga, ctgctttcccctacgt,
ccagtgcaccggtttt, tctaacatcccttata, ttaacattgaagactc, ctctattatcttccaa,
gcagagcaaaaaattg, tgattaagataatggg, taaggtagcctgagac, agcgctataactccgc,
taacgcacttcacagg, ctttatccaatctgag, gcatcctctgtgacgg, taacacctactcctta,
cggatagggggaggct, acatgccggattgagt, tatagaaaaaaaacgg, ggcctaggggcctcgc,
ccagcgttatgcctga, gtaaaaaaaacatttg, tttctgacctacgagg, acctaaaaaaaggcag,
gtaattgaataggttt, ggtagggggggggacg, tgttgtaaccccccct, acatccggtttttttc,
cacctattagtaagaa, ttagctgaaaggcttt, gcaacgaattatgtgc, atagtcaaaaaaatgg,
tgcaaaaaccctcata, gggcagtcagcttgtc, aaactcgtcctttgta, ctgaccatctgataat,
attggggggggaataa, tctgaggcaaaaaatt, tcttaaacatgtgctg, tagcctcttgaatgat,
cccctgtattctgctt, ccttgcctgcctctcg, attgttgcccttccaa, tgaagatagagcactc,
ggcgtggttacacacg, ttagggataccagatc, taagatgcatatctgc, tgatgatatcaaatag,
tggttgagagcaacac, ggcttggtggcgctcg, tctaaaaaaaacgtag, gctaaaaaaacttttg,
gccttttttttatcaa, ctgacttccaattact, tgtcccccagaaacgt, gaggcctggatgcggc,
tttcaatttcaccggc, tgttgctggcagctga, tatacgcccatatggc, attgcatagagccgag,
gtcaatatgttctaga, gagcctccccccctgc, ttactacgaggcagtt, acgaccgatgaaagga,
tttaacatcagattca, ggcccttgtgttgtag, gtcgggacccctgagc, gactcatcttctcacg,
ggctgcactgaaaatc, ggtccataaaggcaag, gctcgtgcttaatgtg, ggtagcatactgaaga,
cttaacccttgctgca, gctctgatctaaacgc, ggccctacccagaggt, acaccccccaaatatt,
gaatgatgtcccgaca, gcgttagggcctcaaa, ttatacatgttatgcc, tacctctagaaggata,
cttatatccacatctt, aatgtattttgggacg, ccccataagatgatcc, cgtttgctgtcagtgt,
ttggtatgtgaactgg, tgtagcacttcgcccc, ttgcggctggacatgc, taagaaggccaggggg,
atgctccttttatctc, agctcctttttggata, ctcctgaagcttacta, ggctacggatgacctc,
gtagcccggctatgcc, ttgggccgggggggct, gactccgtctagggac, acaagggccatggaca,
atgccgaggtcacagg, taacaacgagatgatt, ataaacaatgacactc, gccttttttttgagga,
tcaggacatatgcgtg, gatggttgggggggtc, tctaatcacaccatgc, gggtgcaccctcaaac,
gtcccagcccccccct, aatgagatcagggcac, gccccaggtaaaggat, gaaaaaccgtaaataa,
atcaggcaagggttta, ttacctaagctttctt, acttacttagagtgtg, gggctagtgaacaaaa,
ccatccccccccgaaa, cgaaatcatgctttct, attttgtggtggcgaa, tgctgcgcagctgtcg,
ttacagtaaggtgaag, atcaacagctaggcgg, agtggtatcgagaaac, gtaaagctcaaccagg,
ctatcttaggaccaat, acaaaactatcgatgg, gacttatgtaatggga, cacctttcatagggtg,
cgcccgctgcaatgcc, cgctccatcatgcccg, ccccgtgggtatggtc, ggttgtgaagtgttgg,
acaggaacttgtggtc, gaagcggctggtgcct, agcatcactcatacaa, caggcacggcgccacg,
ttaaggtgctgctact, cagcgattgtaggaac, ggttacctttgcaaca, cgaaatcttgaaagat,
ggtatacttcttagta, gaagatgattgggggg, gggtttgttcctcgcg, tgtacagtgacttact,
tgctcagtgtccaccc, ttaccaggcccctctc, gtgacttaagcccact, gccgcgggacacatgt,
gctatagtgggcaaag, attgtgtggaccacta, aacgggggctgagggc, accaacgattcacatg,
aagccacaagacgagt, cacgctgggctctcta, gctggtttatgcaaca, aacatgttgccgtagt,
tgaagactgttctggc, acttatgatttctctc, agaggctcggggctag, atattacagcatcacg,
tgccggatccctgagt, tacctttttgatacca, acctcttacatgaatc, tgacagtccacggctt,
ctaaccgtttttttgg, gctttttgctcgcccg, tggcaactatgctaca, cacatgtgcatacgtt,
tgaagcctgcccggga, cagtcacattactatg, tccctaactctatttc, gctttcactctcgtcc,
ggtttgaccgcgttag, tccacagtttttttag, attgagttacatccct, tcaatagaactctgta,
tacacgccccttgata, acgtaaaaagaagagc, ctatgcgttttcttgg, ctgggtggaactggga,
ccgtggctgcgcagta, cggggtttgaccgcgt, tcttgtgggacaaacc, tgtgggtatcccagac,
tgaactataaaccaag, ggttgtgatattccag, tccgttgcatggaaga, tctttgttctgctagt,
aggccgcgcagaaagc, gctttcacctcagcgc, cgctttattttttatc, acagggtacatctcca,
tttaaaatctttatcg, agatatacccctgatt, cctcgaatgacttctt, atccaagctcagatag,
ggtgaccatagagctt, ggagctaggctccgcg, agtttggcctcgaact, ctcgtcaaaaaaaatg,
cgggcgaagctcaggg, ctgagctttggtaacc, gactgtttaagccttg, taagcgcgcggggact,
gtataagtttgtaatc, cctaattgtcctgctt, atccaaaaatcgccaa, gcgaggcctaggggcc,
aggcagttattctata, ttataccgtctctggt, gatggggggggctgcc, tagctagaactggtgg,
ttagtaagttctatat, tactgaataaagatcc, acacgattttttttgt, cacctcatagccgcta,
tggctttctcccacac, ttcttaatccggagat, acttgatggattggtg, ttaaccacacttatat,
gtcattgcggctggac, ggttacacccgctgaa, tcggcggctggacatt, tgggaggctcatgcat,
gctattaccaatctgc, gtatcttataacacat, tttacatggtcatgta, gtgagcaccgtgcatc,
agcttagaagctaaac, ggccgtgctttccggg, ttccatttccggtaaa, agagtccgggggaaat,
cgggtgaattgcttta, catggctggtccaata, aaagtactgttgttga, aagccgcttttgtaag,
tcgtattagaagtgtt, tgttttaagccgcaaa, aggggaggctacgggg, taacatttagtctggt,
catactgtactcaatt, ctcctgaagaggtttg, ctctcttaagccatcc, ctggtttgagcaaggc,
ccactcctgtaagggc, tcacgtgatccccaag, ggccagtcacggtggg, actcatctggcaagac,
cccatcatgccatttg, acaacgtattgtgcta, aactgtcacttgctat, tggagcctacacaaat,
tttgagtacggaactc, caggcgcgtttcacca, tcaaggctttttttgc, gcattcaccagtagtc,
gcaagaattaagggat, tttcagcaagtacgat, aatatgactgaaaacg, ctttcgttcttgtgta,
cacgtattccttacac, atcactatgtacaact, ggaggcttattgggag, gatgtagatgtggatc,
cctgtaatccgaagca, actttaatcctggggg, ctcagcgtatcccatg, ttacttaatgtaagag,
atggggggggggctat, ccttcccgcttaggct, caatgttcctgcctcg, ccctccgggagggtga,
ctccatctcgggcaaa, tgtggaatgttagtcc, aaagtgaccttcggtc, agaagatactatatct,
agagttatagaacccc, tacgtgaccgtttgga, atgcgatttgcctgtt, acttcactcaagccta,
ttgcccttcatgtgtc, cgccctgctttttttg, tatagcctcgctggca, tagacgctctgaattt,
gattttgccattgcca, ctcggggaaaaaatag, tgcctgccactccata, gagagtagctaatagt,
agggggggtcctggga, ccaccatcaatggaac, tttacttcctggaacc, tttccttttcgatctt,
ctcgtcaagtggcttc, attaggggttttttat, tggacatctttaggta, cagtccactaactcgt,
agagatcgagagcaac, tttgctcgcccgcccc, acgattcacatgagtt, ctaaagtcactgccta,
cctggactgcatcaca, gggtaaaaacgaaaag, tgatgtcccgacaggg, tgttggcgtagctggt,
agccaggttggccctc, gctttactccttcgtc, gagctggaccagatac, cgagtgactgggattt,
gacgataaaaaaaatt, ttaaaaaaaagcatcg, gatatctcctttgcac, tgagggggggttgttt,
agagggctgtgggtac, ttgaagcaaacttagt, tagtcctagcactagt, gcctgataaaaaaaag,
tctcactacaattcct, ctggaatgtattgtgg, tcgcattgacctttcc, ggcttttcccccccct,
tcttgatccgcccaac, cacattagctcattcc, gttggtgctagtttgc, actaaggcagcttagg,
atccatgccaagtgtt, gtgcactacagtagaa, ttggcgagttatataa, ttactaatgcacgcag,
ccaaacgtctgcctgg, tgacacgggctgtttg, gtgtggatgttggcga, tcggtgtgattttttc,
agaagatccaggcttc, ctcaaccagccactgg, atagccacccagatca, gttgttagttgcttca,
actgccaccgaggtgg, aagcttccgcactctt, ctgctacctgcatcat, tcacggccggggtagt,
attgggaaaggcccgg, cagcagcctagacttt, cgggtacaccaatcac, aagtcattgaggctgc,
tgtcccccctagtgaa, gtagcgatgctgtttc, tctacttatgaaacgc, gggagactgtaaacta,
atagatgggttcttgg, gttatgtatttgagct, tggccgtgctttccgg, gactttcccaccacta,
gataggtggttattct, aactacggctgcattg, acactaagtgctgtac, gagctagggccaggcc,
ggtgcagcaagttgtc, agccaagttatacaag, agctcatggctgcaag, ttcacgagtgtcaaat,
ttatcaacaaactcat, accgctgagtgtcttg, ctcattgtaggagtgt, ttggctccgtccttac,
cgccacaaaaaaagtt, tttaccagactcaggt, ctcggacctgttttca, tctgatggataggaag,
aatagccacccagatc, cgcactgctggatggt, ctatatgcaccattag, cgctaattgtatgtta,
gttctagaccttgagc, agcctccccccctgcc, aagatgtccatcattc, cagttgatttgtttta,
tgagcataaccataac, gttaaacaggcctaag, cgggcagatgacctta, aggacgaaaattggtt,
ctggacatccgccgct, actcccaataatgtgg, ctattaatcgtccatg, tttgctttcgttatgc,
caggaatatgatatgg, atgcacatttagtcag, attccgtactgatgct, gtgcctatcattctga,
aatcataagtcggctg, tccgcactgccccaaa, cacttacggagcatat, caggccttagagcgca,
cccagacgcctttgcg, atgataggtgtctctg, tgagtgtaagcttgag, aactttggggacaaac,
agccatgaagttaact, tctattcgtgcacagt, ggcccttccaggagcg, agaaaagtgttcatgc,
accgcgccctgccacg, ttactaagctacaatt, ctgtgcttagtttgtg, ctgcagcccggggaaa,
aggttggggggggaac, attagtggtttactga, gagcccaggaagccta, aaccatttttttagca,
tgtgggagtatgaagc, cccgtcccccctcttt, caattatcgtctgtta, ggactcatgcagtaag,
tggataacagaacagc, acaccaactcatcaaa, aagtaagagacagctt, taagtgttcgttagat,
gtctctcgcacagcaa, caaattttaataacgg, ctcagtgttatctacc, gacagccccccataat,
taaaatccccccccag, taggttagaggtccaa, cttgcgccaactgcat, ttgttgttctccggat,
atcattgaccactaaa, gtcgctttttcccccc, acggggattaatgctg, cgctgggttcttgatc,
ttgaccaacaataaat, tcacagcatagattat, gatctgaggggggcag, tactgtgggtggccat,
agttgggggggaggaa, tcatctgatagaacac, attgcatccccccaca, tggcactcttgtctgt,
cccatcatgccacgtg, cttacctacttccacc, gtcggttttttaccct, cactgagagggatcag,
aacacggcttaaaatc, agagggatgatccccc, agtgagcaggttaaag, gcaaatgcagacacct,
gtgctaggctgcacag, atgccaagacgtgacc, cccgtctgtgacacca, tatacccaacggactg,
tatgcaatcacaaaga, accgtttacttaatgt, tccacagtcattgcgg, gctacgtggccccact,
agtagcctattgcctt, atcatagccttggact, tggtctcttcgtcggg, ctatcgttgtttttag,
ttctttgaacaccctg, gtatgccggcgctttt, tgtgcctccatgaatc, tatggaaagcaacccc,
atgttttacggagatg, ggctacggggaccgag, ctccctttaattatac, atggaccgatttgctt,
ccattgactttgtttg, gttggatttgttatga, attagagcaaccatgc, aatcaaaaaaaaacag,
ggggatcattattgtg, gtaatggtgcggtgtc, gagatcacgtcttaaa, tgttacattctgactc,
ctttgagtcagctttt, gctctacattaaggtg, ttacctcgggatccgc, gctgggctctctacaa,
cactccaatcccgttc, tcgatttaagccagga, agcggactgtacatgt, gtgtaaggctaagtta,
ttcataaaaacgagat, ccccttagactttctt, attcctacccctgttt, acacagtcggtcctct,
ggacaataaatggtcg, ctgaatgttattaccc, aagcatccttattgag, ggtggggcatagaaat,
acaattattccaccga, cagagatgcctaaccc, cacacctgccgacctt, cctcagcgacacgagc,
gacctgaagaggacac, tgcctcactgcgacgt, gatctgatttacctta, ttggatgagataaagt,
ctcggagctccgcgcg, ttatcctaccccccag, tctttttttgtatacg, ttaggcgggtggttca,
ttccttgcaaaaaacc, tgtacttacataaggc, atgacatgcttcggtg, gcggaggaaaagcgta,
gattcttgagcaggcc, cgtgtagctgaggtgg, cacagtacgtggttct, gtccccaactttcttc,
aagccatgtaaatggc, attcacttttaggcta, ccttttttttgcacta, ttagcttcgtggagag,
gcctcctccgtttctc, agagctcgtctcctca, ctcattaaatgggatt, cgtttcccagacgcct,
ccggggaaattctcag, catcctgtttccccgc, agccattaagatttag, acaccttcccattggc,
cccatcaccttgtcat, caccgaggggggggga, tgcacgtaaggagtca, ttcattcgcgtctgtg,
gagcttcgctgttatt, actctcatccaagtta, gaaggccgcggggtac, tttgcccaagagcaaa,
gaagagagacctctag, agtaggttgtgaagtg, ggccccttatcttaaa, agctaccatggcctta,
caattgcgtacatatt, agcgtgtggcttaacc, tttaccttagcttcgt, ccatactatgtcgaaa,
caatgcctcgatcagt, gttaatgggttgggca, gggggccctgtaatgg, cctacaattcatctaa,
gaaaacatccagccgt, attattcccccagtgt, gtaatgcagggagatc, gggctgcatcacgtga,
cttagcagagcaggta, cagtcattgcggctgg, tcaaggggtttattcc, tcaatttgagagatgc,
cctgatgtaatgtgaa, taagacggcattttga, ctaataggggcattag, tcagaggctatccctt,
cagagttgagctcctg, aaaagcatagaagacg, agaccacaaaacttac, gagtggtgattactct,
gctctcccttagcaaa, tcccggcatccttgag, gacgcctttcatatgt, cccatgaggtacagtg,
gcgtgaccagggcagg, agccggggatctctgt, gtgcaccctaatggcc, atgcgcctcccgctat,
atgggcttactgtgca, ttagcctcatgattcg, ttattttacggatgat, caggatgtcctgtctc,
catgtagtcagaatag, gttggcgtagctggtc, atcattttccgaactc, atagtttagtaccacc,
ctggcgagtccaacgt, gcaccgtgagattata, ggcgcactattgctgg, cacggctgtggtgcta,
ttgcgggtgagctgat, tgattcgcccagcttc, gcgttctcacaggatc, atgtacccacctcggt,
ccacccacagtagcag, gtgatggcgtggactc, agtctatgtattcccc, catcctaatagtcacg,
ggacagttttcaatta, gctgagcgccgatctg, tatgttctcgaccaaa, catgagcgacggagaa,
agtccaggcttttgtc, aaagtcatggggggga, gcttgtacttcataac, catggacaagttcacg,
gaaattcgggccctag, cagacgaaaaaaatca, tatgcgtaccattttt, gtaactcgtgtaaccc,
taaagcttaattaggt, tacttatgtactcagt, agatacctggtacatc, ctccgccttactattt,
ttgaagatcaacttcc, gactgcagatagctat, agctcgtggacaaaaa, ggtctcttcgtcggga,
gtaaaaaaaggaggct, gctgatccgctgtacg, atgccggcgcttttca, ggcatatgtaagaaca,
tcgctaacaggatgaa, tggctaattaagggtg, catgttcttggacctg, ggccgggctctgtgag,
atcctattatcctcca, atgtgtgaagcaaatc, accagagatgacaccc, tcataattctccagag,
cacagggccccccgga, ggagaagcttactgat, gcgaggcttccacaca, ccgcaaagcaatagtc,
ctactgttcctcgaaa, tcgctattttttctgt, cccaggacccgccgct, ggaggggttcagaact,
ctttatacagttctgt, ccgaccccaaatatac, cctcgagggtcctegg, gcataaaatgagtaac,
gcagggaataccttat, ttttaaacacgagatt, tagcgtccgggcacgg, gatccccctttttgcc,
cccccacatggctcgg, gacagtggtcctcaat, aggggccggggttaaa, ttgcgattgaagcaga,
acatctttcttcgacc, gaacgcaggaggtgta, gatgcaatgctgtcac, tgctccctcggtcatt,
gttggggggggaacaa, cattagtgccatttgt, ccttcacaatagttta, tgtaagctagttcacc,
ctaccccccccgaaaa, gcgtccccgtgggtat, cccctacgtcatcctc, agaatagacaatctac,
tcatagatggctaact, ctccatgtatcacgat, tcggggtttttttagg, cctgttacttttgatc,
gtctggaaactggtta, tagtgctaatgatcct, tacggagtaaactaca, acctcataagaccact,
aggatctaccttgtag, ctggaatctagaatgc, gacctatccaagttta, ttgtaagactttctgg,
ggtatcctagaggtaa, acgcgcagtattctgg, agaacagtcccctgag, cctactgatgataaga,
cagactgtttgctgca, aaacctagctccttgt, tatgagccgcccccaa, aggccaacaagggcaa,
gaagcttttttcgtgt, tcccccccccatgaat, ccaatataggtaacag, ggagttgccaaataga,
ttccctggagcttgta, aatagatcttactaga, cagtagactgtgggat, tttataagatagagct,
gccacgagacagcggg, gggcgacaggagttaa, cataaggggagagctt, gtgacagagcgttact,
tcaccggatgggctta, agtttagtcatgcagg, ccggtctgaaaaaaaa, tgaagtgtgcgtctgc,
agcatcatatgatgac, atcaggcacactttag, gcagctccagaatccg, tgcatctaaacgagcc,
cattgatgctttggct, ctggttccacctaaat, acaaaaagaacggaca, tcctgtatagcatatg,
cagtggccttcctaca, tgaagaccaagaggcc, gcttccccccccattc, tacagcttctggagtg,
cgatctgcccagctgg, tcgaaattagctgtcc, gatgggcgagtttctc, catgtaccacaggggt,
cagtttgactggcaat, cgaagctgagcccgta, ttggcagggtgcagcg, aaatcgtgtgctgtgg,
tggtattgcaggtgca, cgccccccatggacct, tgcagtaagcaaaatc, acgcctgggggggggg,
tctttacatgtccttc, agggccatggacaagg, tcaatggcatgcaggt, cccataaaaaaatctg,
gggcccctcgacgcct, gcgttcagaattctta, tttttctccgttatgc, cattgagcataaacca,
tcttaagtaaaggcca, accettagaggccttc, ctgaatgacagttata, tgccaccaagtcttta,
cgagcttcgctgttat, tggtacagaagactgc, ctgatctctacacggc, gacgtgaaatttctgt,
aatcatggtaaatgga, gccaaggttgggcaag, ggcgttaaccacctca, aatgtgcagacagcca,
ctctgataactcttag, acgtcaatttttttgt, tttggtagcacaaatc, ggggaactgtgctctg,
tagcctctccaatttt, tcccgacagggcgggg, aggtgtaatatcccga, gatggccatgcgcatc,
ccttattttagtataa, ttttcaatccctaatt, agtatcacaacaaaac, ttctgttggttaggat,
ctgccccccccggggg, tctcgtaatctactct, ccacctacgtcccttc, gaccactttacatcat,
ggaatccctttgcttt, ctcactcggcggcagc, aaacgcaatttaaaaa, tgggcgccagagttag,
cctcgcccggccactg, agatgacttaacgcca, ggtgacaagaggttag, caattttcgtcttaat,
gtggtcgcctctaatc, atttatagcttggtta, tcttatcggcaaacag, ttgatactttagcttc,
tttggatcagaaagta, gaataggaagaaggtc, atagactcaacatcag, ctccccatagtgaccc,
cccggcaggtaccact, ttatttcctcgttctg, acatctttccccccag, atatctggcacgagga,
tcaaaatgcataacag, caccagcgcttcatgc, gtggactctgtatctt, tttcattgtgagggtt,
tacctgaagctcggct, ttctccttacaagctc, gcgcactgccgtgggg, agaggaggttgcgaaa,
atataaagatatccct, agtccctggaccttcg, gcgtctgcagttagct, tagggggggtgaaagt,
tcctgtagcaaaggtt, catcagcggttcaaga, tgactcgtcacccagg, gcggctggtgcctgct,
aaccctcctattcgac, ttatcctggagcataa, accacagacagcacgt, taatgtggtttggagg,
ttccatgtattcgttg, tccgtgactctctcga, tggggggggaccacac, catcagctgctcgtag,
gatgccgagcctaagc, caagaatcccttgaga, ttctcttcacacggga, ttgttagctggaatgg,
tcgcatgtatcttttg, ttacacagcgagactc, ctccgctttacaacat, acacccgggaggagga,
gcggctgggatcgaag, cttccccgcttagcgc, ctcctattgcttcagg, gttgctacccccccaa,
gtgttttatagggtga, tgacgtacataccata, agagagcatgaactta, cgtaaaagtaattgct,
tacccaaaaaaaactg, cttagaagctaaacat, atgcctacccttcctg, aaggtgcaagcctgaa,
tatgatagctactacc, gtgaggggggtgttct, aaacaccctccccccc, tcaggtatgaagtacc,
caaagcgactgtttag, aaatggtgtgacagac, tagtttagtaccaccc, ttgtctaggcaagact,
atgtgcagattagaat, atgttctcgaccaaaa, gcttacacacttttgc, aggttcctatacttct,
tgtctaaagtagctta, cgaaagtatgtacata, tacttagggaatcagg, ggctctcgccccctgg,
tcccatctaggacgtg, gtaaccgcagtgggag, tccatcttggcgacag, gtgaaacacataacct,
tgctaaaaaaagtggc, cactcggcagtgctcc, ggacctgagctgagat, atgcaaggcggaacta,
ccggtgctgacgaacg, agttatttaccaccga, atatacaccgggcctg, gagtctctcatagttc,
tttgcaaaagggggac, gggaggccgcgtagaa, gatgacacctagcttt, tccgtcacacaaagaa,
ggtaattgactggatt, gtcctggtatctggat, gttgttacacccttgg, gactacaggtccccag,
ttgccctcttaaagtc, gtggctgcgcagtagc, ttgaagaagtcgattt, actcatcatgtcagcg,
ccatcagatgatgccg, gtagtagcccagcagt, tcaaaggaacttattc, attcaaacaccctccc,
gcccaccacataagcc, cagaccgggaaagatg, aagcggtgaggatgca, gttgaggccactgatc,
gtgctcaggcacttgc, atgagggggggggggc, tcatgcataatggctt, gtcactgtgctcgccg,
tgaaagatacccttaa, catctctgagtactta, cagcatttttgcaggc, ttagtctgttggtctc,
acggaggggttgggat, ataaaactgtatggca, tgtcggtgagtggagg, aatagtccagttgtaa,
agtatgggggggtgtg, aacctggttcagcgca, ggttgagcaacaatga, acgctggaaccacagt,
agtgggggagtgcatt, ttagcaacagaacagc, cccagcccccccctcc, gaaatttgatctatga,
aacccttttttttcag, ttgagaggggacaggc, gattatgaaacatagt, atgacctagctgttag,
gataatcaagaaggaa, ctaccacatacgtggt, gtaagtttaattatgg, tctcgaaaaaaaggag,
gtacgctaaacttaaa, atcagtgatgggttgt, ggtctctcttagaact, ctatacctcaatgagg,
tgctaggctgcacagt, gcaaattgcgcagtct, ggggtgcatcgtgctg, ggactacaagactgtc,
gtgcactttagcaccg, actttttttaggttct, gaaaaaaaaatcgctg, ggtgtgattttttccc,
aatataacacctgggg, agattgcatctcaata, agcacgtagaaatttg, catgcgtggagagggg,
cgtaaaaaaatacagc, ccacacccaggatgcc, catttgttgccctgtt, tgatatctaactctga,
ctatcccccccatttg, gcttaggctggacgtc, ttattgaaatacgttg, atgatacgctctaaga,
cttccccacgtcagat, tatggggttttttcca, ctgccgtgccggatcc, cctgagcctcgtgagt,
agttgaagactatcct, agttgtggtgtcgggc, aggtgccatccttcac, ggactgcagtgtcgca,
aaattcaggcactaac, gtccacgctccctaga, aagtctttgtctcgct, aaagagactttatcct,
tgggtggcttaggcgg, agcaacctccttcgct, attctctctgtttatg, agattgcccccccccg,
ctcccctagttcacag, ccagttggcaaaacgt, caaatcgtcacagcag, tactgtttcctggcta,
ccctggtaatgcgatt, cacattgaaaatcatc, ttggggggggatgctt, ctattttttttgatct,
tcattattcccccagt, ggactttaacctgata, acccttagctcattca, ggatcgctgagtgcca,
tctgcagattagtgac, ggaggcaattctgagt, ttacacaagttattcg, tttttcacacaggaac,
agcctgattgcctctg, cggccttgccgcgccc, ctggtaacagacacat, aagtgttcgttagatc,
cggcactcacacctcc, aagttctaaactgact, atagtaagggcataga, gatacgagtctccctc,
cactgccattcgtggt, gagccaactaattaac, ggggtcctcgtgggcc, tgggattaagcaatgc,
taagtcaactgaacct, gcttcttcgtctcctg, aaactactgaccgcaa, tattgctcaaccccca,
ggctgtcccgcggggg, ttaaagcagatcgagg, cttgagggtcgcatcc, tgtagttccccagcac,
cgtagctctttttttc, agattagtaagtccta, accacccaataaccca, gggggggggctgacta,
gcctatacaagtttcc, gaccctggtaggctga, atgggctctatgggaa, cttagggcattgcctt,
catagacgtaaaattg, atccatgcccccgaaa, accctttgctactgaa, ggaaaacgtttccact,
gttaggatgctgttca, ttaaattatatcgaga, gtggttcggcaagaga, gtaccatttgcctgta,
ctcccccgaaatttct, tcgcaaactaacaatc, ttcgtaaacctaagcc, gaactgggtttcaccg,
ggagtcacgtaaatgt, ggtcacctccacctct, acttgccaccaatgta, ccaagaattgaatgtg,
gatgcctggtatggta, gggggggtatattctg, ctgggttgtgcaagat, ccccccgccagcgcat,
tcctgaagagaaagtt, ggatgacacaaagtca, tagcatctttgatacc, atggtggttgtttcgt,
gcttacttaatggata, atcaatttaccttagc, tggcatgcggatcact, gtccagtgtattatga,
cctaaaaaagctgccc, gggtagcagtgacctc, ctgcacagctcgcttc, atctcctttgccctac,
gccaccacgcctcttt, gatatgacctggaata, ccttctgtcatgaacg, ccctttttgggcctcg,
acttgacggttcctgg, cctgatgcatagtttt, atttcactgctcacat, actacagatttccacc,
ctttgcggggggctcc, gttaaggtaaggtaaa, aatcaaccgcttttta, acgggactccatattg,
tcggctttttattact, aactactcactcagct, ccgctgtcttcaccca, ccccctggagagattt,
ggaactaatagtattc, atacatgtttttagcg, ataaatgcgtcacaca, cagcaatacacggccc,
cttgtgtaaagcttaa, cacccactggtagaag, ttgtcccccccatgtt, cctcaggtaatgtacc,
cccgaggcaccctccc, taaacgcaaacaagag, tgtagtttacctagtt, tctgagtgtaggtgac,
ccattgctcccccccg, ctctttggagaggata, gtcaacagcggaaatg, ctatagtgggcaaagg,
tggcaggggaaagccc, ggagttttttttgagt, tttccccctcgtgcaa, ctggcaatgcggtaaa,
ggttatgccatcagtc, gctgggcaacggtgcc, gcttgcaggaacaacc, tcaatgtataatttct,
aatacctaactgagct, actctgggcattctag, tcacccccacctgaat, ggcgttatctcagctg,
tggcatagtgccagtc, actcaccatctcatac, gcaaggtatttagtga, tgatgacacaatatga,
gtcatggtcccttcat, ggatgtgacgagaggt, aatggcggtgaagttc, tctatgatgtaagcct,
ggatatggggggggca, aatattggcttaggta, taattggcctgaactg, gcttccagaccactta,
cccacagccttggtaa, agcttgcaccccgaca, ttagggagttgatacc, aacaagaagaagttgt,
gttgttaatccttagg, agtgaccccgcaagga, ttcgtaagcttaggat, ttctgaggctcgcacc,
ttacgtatataaacat, acaactagaacacttg, aaaggaacaccttagg, gccagtcacggtgggt,
aacttaattcccaacc, tgtgtcaccgagacca, ccagcgtcggccccgc, cggggctgagctcaca,
tggctgtctgcttact, caagcgtaaaaaaaat, gcaccagtaagcttaa, gcctgtgcttctttat,
tgccaaaatacctgtt, tcccttatacttcatc, ttaggtactcatagat, gcacacccagtgggca,
gggaggtgtagcttac, gtactgggcagtaaaa, gtgaaccgcaaaagtt, caaacaagagcggtga,
tttgtaaagggcccta, ctcaccctccgcaccc, ctcacccggccgagca, ctggatgcggcagccc,
tctggctgggttgtat, cgttactgaggtgctc, caggtcagattgagga, accaaagttgtgggtg,
gcatgacgaggtcctg, tccccccccccttggg, ctccagtgtctccgcc, atttaatggacaccat,
cgattgtctcctgtgg, ggcttgggggggggag, ttgaagtatacctagc, ggtgtttttactcagc,
cgcgcagtctctgtat, ccttatctgcttatga, tggttagcacttatcc, ctccacgtttttgttc,
aggcccttggctaccg, agatggatataccccc, gaacacttccatatca, ttaaactacgtctcaa,
ctgtgccccccgaaaa, tacacagtctgggtgc, ttgggctgggattagt, ctctatctactggtaa,
ttcacagctaccagcc, gggcgggtctccgact, gatcttagaacgggca, ggtgaagggctgtggt,
caccttgctgcttggc, acgttactagtctgtg, ctgttctctaaacccc, cggggtctgatcaagt,
acacatggagtaaagt, ctttcccaccactata, gatagtgtaaaaaaag, tactaggtgtgactgc,
agcttcaggggttgtg, cggcatgtagaaatca, gtgtatccaattacaa, aaatctgttgttgcga,
caagacttagtacctt, ttcccctcttaggagg, caagtctttgtctcgc, aggtcaggagactaat,
gctgatgaatcaacga, atacagtcatggtgaa, ctccgacgccggctag, cctcttttacacatac,
aaccctctgccgggcc, aagctagctacaacgt, tctgtagggggttctt, ccaggggatacgtggg,
gcctcactaaacatat, tacccaacggactgta, ttacgtaaaaaaaagg, ggtccttccgggggcg,
gccaaaaaaacaagtt, atcccatgattattgt, cagtggacccccccat, attcatccccatagac,
ggctttaaaatcttag, ctattcgggagactta, attttcgggggtggaa, agggggggggtcctgt,
ggccttttttttaagg, gattcaaaaaaagagc, cgtgaaatgtcaggtt, taggatgttataagag,
cacgccccttgatatt, tattgacagccttata, tgaccatacggtcaaa, tgagcttgatgtttga,
gaggttctttcttatt, cggttgccgtgagctg, ggtcgggggggggcat, ttgagtgaagccccga,
tcacggtcacctctgt, acacatcaaggactct, atacgcccatatggcc, gacatagtgtgcccct,
tgtgagagttaccgta, agcagggggagcttag, aatttatcccccctgg, cctcacccggccgagc,
tgtatgtctcttgcga, taagtcctatcacatc, agtcaagatcttctct, atcgccaaggagagaa,
tgtggcacccacttga, tgcagagtgcggattg, caggtcaaaaaaattg, gcgtgtgggaatgctc,
ggaagaggggtagttt, ttatagaccttccgga, caggatgcgcagtgta, tagtgcctttggagtg,
actgttactctagcca, cccacctcagcgacac, tgcccccccccagtct, cttaaaacttcacggt,
gcctgaagcaccaagt, acttttgcactcggag, gttatcagcacttagt, cagctgatagattggt,
acatccataggagccc, cagcccgtccggtgct, ctaggctccgcgaacc, gttttaaggggggggg,
ccgatgggggttgagt, gtgggtcggtaggaat, ccttggcctgcgggaa, tctggagttccacggc,
aggacttagacggggc, gattttttgtcgttat, gactttgttccttctc, attatgaatgctgcat,
tegaaaccgtctgaac, taccactaccaatgac, agtgctgctatatgtg, acgtttttcctccagt,
tatataagcttgtagt, cctggtccatttactg, tccgacgccggctagg, agacgagggcatattt,
ccacgatggtcggcta, gctagtttcccttgtt, tccctatgatgccaga, catggtaaaagctaat,
cgtatatatatctttc, gaatacctgcttggtc, ctctgtcctacccggc, tttgatattttggacg,
atgtgagtcacgaact, tcgttatccgctcgcc, ctactaaaactcatca, gatgtaagggtttttg,
tatttgaacaacccct, tacaccctaaaaaaag, ctaatgctcccccccc, ttccggttcaggacaa,
cgggtcctctgaggtt, gggttgtaatcctatt, gacgagcttagagaag, acgccgtcccccccgc,
ccaattttgtcaccga, ggctcggaagggaaca, ggctgcataaagtagt, ttaagtgttcgttaga,
tccgagccaagtatgg, atgaattgaagattct, tcaaagcccccgaaaa, aaaaacgcattaatca,
cggcaaaaaaaactac, atcaaagtatagcaga, tcctagctgaatggag, ggctacccccccatac,
gttgcttaaagccccc, ttcaagattggagatt, agataaagacggtttt, gaggcttttcaggtca,
tgaggatgtcagcatg, gcttcaacataagcta, ctttaccacagatcag, accccccccatacttt,
cggggccgtttttttc, acgaatacaccatgtc, gtactgacttgccaga, cactgacactccttca,
ggggttgaacaccagc, ttctaatgcttgtaac, tgactctgcgccaggc, cggctgaagagttccg,
gcttatggtctcgctg, tcatacggggtggaag, tacactcttaaagcta, cacatggcattcggcc,
tccatgaaaaatacga, ttccttcgggctccgc, agacctgattttttag, gttagctaagttgcaa,
aggttgagcaacaatg, cccagatgacttaacg, atgtcctactttgagt, attcctccaaatgccg,
ggcgaccgagactcct, gcgcttttttttcagc, gagatgagagtgttct, tctctttttttcgacc,
ggctattggccgacag, gactgcagttatccgg, acaagatcaaagatgc, atggaggtcttagcat,
gcctgagacattggtg, cactccgtctaacaaa, ctctcgtctgaggctt, aagcttttatgaggag,
ctacggatgacctcgt, gttactttgcacttaa, aggcatgtgaccacgc, tcgtaatttattttat,
cgagttattaggggca, gacgagggcatatttc, tctagccttatccatg, ctgggttcaatccaat,
gactgaataggtatgg, gaattaagaatgtcgc, tagggttcccttactt, actcttagaacagaac,
ttatctgtaaggcaga, tatgaaaccaacacgt, aatttcaccctaccta, ggaaatcattttccga,
tttgttgtgcctggaa, tccgtcttctcctctt, ataaaatgcctcggct, cccacaccctgtactc,
actccccttatcgcag, ctgggaatttcgtggc, ggcatgaacatttctg, tttgtgcgccggtctc,
acaccctctccaatgg, tcatgtgctttggctt, ttggagggttaggctg, atgttttttttacgca,
cgctaagttttttgtc, gctggcttattccacc, tcgtgtagctgaggtg, catgggcacccacact,
gggtaaaaaaaaaacg, tgtggtgctaccactc, atgactaagcatagat, tcttgtttgactctcc,
tcacatcgctaatttc, tttacccccccccgaa, ctccatttagatacgc, ttctatctcccccgaa,
tacgccggcggctgag, aagctgaaacgagaat, acaaccttttttgctc, gaatccacaccttact,
cagtgatccaatatcg, ttgggttcacacctta, tttgatatacgtagta, atcccagttaggttct,
tagtgtttctcattcg, tctcttaattgcgatg, taacaagaacacttag, ttccccatagggtgtg,
gagaccttttttttgg, cgtcttctcattcgtc, tccaattctaagttga, catgagagaaacctta,
actcgtaagcactcag, gtaggagccctgttgt, tgcacatataaggatg, aactgggaaacgaaca,
gatcatttccggaatg, gtgatagtgcctctac, gcttcgtaatagtatg, gctgccagtcagattt,
gccattatgttatggg, gggcttcttaggcgat, caccttgagtggtgat, cggccatgccaatgcc,
ctaggagtatctttct, gagaaaaaaaccagtc, cgccatcgcgtggtga, atcacaagcctttacc,
gcgtcctctgtctata, gcttttttttaacgtt, agataaaaaaagctca, gtaagagccctcggtt,
gcttccaactttctta, caagcatgaacacttt, agatcaccttaggtcc, ggctgctgataggtta,
tcacggaaaactccgc, agacgttacatgcatg, aatgaaagccccatgt, ccttggacgtgttgag,
gccccggtctccccct, cactacatcatgctat, caccagagacgcccca, cgcattgttcggatga,
gcgggtgcccctctgt, aggcaggtgggcggtc, cattaataagaatatc, gagacctgtgccgcgt,
ttctttatgactgatg, ttaagatccatctagg, gcatgtagccctcagg, gaatacgcttaacaac,
cactacagatgtggct, tcttggccgcaacctc, tttaagttagttcaaa, taattttgaacggaaa,
tttacgatgtgaatgg, cacagcatagattata, aggctgcactctaact, tacaagtcctcttatg,
gatgagaggattcgtg, cattttcgtaggccca, gatattgtccaaggca, atggacctggtaccgc,
ttgcccatcgggaagg, aaccggggcccataga, atcaattaatgggcca, tcggacctgttttcat,
cagattatggcgtctg, aagtaattagactgtt, agacgctcaggaaatc, acgcctttgcgggggg,
agaggcataagtttat, aggtaccactgacggt, cgtatcactatcagct, cctgttttcagaacca,
ttggttctcaattaga, gctttactgcaagacc, agtgagtagaccctct, actgtgtagtttacca,
atgaacaagtctaatg, acaagagtcaacctaa, agggcttaggataact, gattaagaaacagtct,
cccgatatgaaaattt, ctaggaaaaaacgggg, ggattattgagtgtgt, tgtatgaactaaaaac,
gtgcatgccccttatg, tcctagatccttagtt, gtttattaggtgggtg, ggggatacgtgggaag,
tgtgttataccgtctc, accgaatttttttgag, ggtgtgtccagagatt, gcagtgaaaggttaac,
agccttgtaggttcta, tttgcaaagcacaaga, gagcagggaatgcgcc, caattaggatgcagct,
gattccttcaccaact, gtgcatatctgatttt, tgcattcaggcagtta, agtgataatccataga,
gccacatgtcttaagt, tgtggaacgtccgtca, aaacctgttagggaga, gcatcccaggtttatc,
gtggctcatgtagaaa, tccagagtcttgttga, gggaaagtttagtcat, gcaaaaaaaaggtgta,
atccatgtccatggac, tggtgcttaaatcaaa, ctacaactccgaaaaa, cactaaggcaatacaa,
actcctgaggcactac, cgcctgcgtgtacctc, caaacaagtgggtaat, aggtatgcacctactg,
acaaatcgtcacagca, ggtacaagcggtttcc, tatgcttaacttccct, cggagtagacaaatac,
aagggacttatgagtg, aatctttgttaagaag, acgtcccataccccca, ctacttgacgaatagg,
atgtcacgggactcca, catgtctaatgatgtg, cgccacagctaaggtc, cttcttatttcgtttt,
ccaggcttgtggtgcg, gtcttaaatattttcg, actcatgccagccaac, gggctgactatgtttc,
ccccccccatatgctg, acctcagcgcttctct, tcagtaaactaccgta, cttcttaaggctgtaa,
gctcggtttttttaag, tagcctattgccttaa, cgactgtgtcatagtg, tgtgctatattcctga,
cccctggtagaagcaa, cacttattatttgaac, tgagatacagctgtca, gattgacaccagaaaa,
tcactgacactaggtc, agatttatgtgccatc, ggtcaagggactgctg, aacctactcagctttt,
caatttgtgcttattg, ttgggacttcgaggca, atgtggcctgattatg, ggactctgtagcttag,
gagcaggtccacccgc, caagatagcgtagctc, cctgattcctctgagg, acactgaagcgtgcca,
agcgtatctttttatg, cagacccggatagggg, ttgctcatctcactct, gatactgccgtatttc,
ccaggccatagtgggg, agacgtagggtttaga, tgtcctgtttggtcat, aatgtccagtgtggct,
atcaagctccgatgaa, atcgtttaaaaaaaag, agagagcgagcaactg, tactaaggtcttttca,
gccactcccattgggc, gaaggcaggtcctgac, aacttagctgtaacaa, gactctcccccgggag,
aggtgaaaaaaacgag, gcatccaccttacatt, agtggccagggaacgc, tcacttgattcaccca,
cgcgaggcctaggggc, tgtcttcggggtcccg, tgataccaaaaaagct, cacatctgacacagat,
gttataagaactgtaa, tcagcggtgcggttgg, ataatctaatgcctgg, aggaactgttgcctag,
agtgagacctgtgccg, tgtgccgagcctctga, cactccactatgtttg, gctaccacgtgcttct,
ggaggtggtccatact, ggaagcacactttatc, tagcatggtaagtact, ccccttctagcatgct,
ttaacccatgcagttt, gcagagtaggcggggg, gttaggtcaagggtca, cttggtcaaacccagg,
tatgttggctcatttc, atgcccttataccctt, ttgcaaaaaaatgggg, caggtttttgtagaac,
atgtaaatgacccata, tgcattgtttaagatc, agagaatgattgtcat, tatgcgtgtttaaaaa,
ggtcagttatgtaatt, tgctctcttagtaccc, cctaagtcctgttagg, acttatagctaggcaa,
gccgattggtcctttt, aattcttgcgtcttcg, cctgtttgcccgcagg, tccggttagagggaaa,
gctggaatgggtttat, tcttcattctattgaa, agggcggcaattagat, gaaaaaactcgtgtta,
ccttaaagtcattgat, tattttttaggggggt, agctaatagtacaggt, caacgcccaggctcaa,
ttggcaatgtagggat, actccactaatacacc, agtcccaatgagaacc, caccactttagctagg,
tctcactacttatgaa, cgattttttttccagg, cttttagtgataagcc, ccaatgttatccttgc,
agtactggagagaaag, acgccttgcagctgcg, ttcaagctgagccatc, tcagcgacacgagcga,
ctttacacctgccact, actgcattgccggatg, ttggggctgcggcagt, tcactgtcggctattg,
aaaccttttagcagtt, gctgggggagtggtta, aggtgtctctgcaaca, aggcactgtgccatta,
cccctgtttatccagg, acaagtccaaacaagg, tgaggtccagacctga, acccccgatacgagtc,
cttatcaaaccctgta, aacaaaacaattaccg, ctgtcactccggtgac, caccccaaaaaaacat,
tattaggttctgtggt, cagagggaacgcaggc, ttgtaactatgtggtc, cttcttttttctaggg,
cctgtgagccgaggtc, gtcttaagtgttggac, cggttactgcggcttg, acaactcataagaaat,
ctaaagtagctcacta, atcccagtcgtggtgt, cttctgcttaacaggg, gcagcttgtctagcac,
gcgtggttacacacgc, attcctgacccagtct, tcacttagttaatatc, tgaacactgtccctta,
agtagatggcgctgga, cgaacaaaaaaaagct, gcccagactccagttg, ggaacttttgcactcg,
aagatttagaaacccg, gactgaaccttagcga, ttgttagacagagtac, tccagegcctacttac,
cctagtctcttcgatt, atttagggatcagaga, tttattggggtttgtc, tattgcacacagtgct,
accacttgctcccacg, gcggagttttttttaa, agccgtcgtcctcgag, ctctccagtgtctccg,
catacatggtgtggga, gaaaccgtgcagtgta, ttgtagggggggacgg, gaaaaaacggggattg,
tctatgggcgtaggcc, accggagaatcccttt, gccactgcggcttagc, caagctttacttatca,
acagagttatatcatg, ggtgtgttgaggaggc, gagtgttcagtaggga, cactgaaaatggttgt,
aactggcaaaaaaagc, cgtcagttataagccc, tatcccattggaatgc, atggacactaattctg,
tgctcagcattccatg, cggtgtgaggagaagg, ttatgagccgccccca, tgtataggtagagctt,
tgcaactgtgctctta, aagccatgccccgtgt, atctacagtctattat, ctacttatgaaacgca,
ccattacacaggtcaa, atctcccttggatctg, aaggtgtccccccaga, actccgactccggggc,
ggatagtgctactcca, gtgggctgtcagcaat, tccaatatggaaaatc, tgagacggtctgactc,
cacaatacgcccggct, caagagccccccccac, gcaaaaaaaataggcg, gttcttgacttgattg,
actgcggcttagcgcc, ccagcactttgtccct, ggagaggggggggaag, tggtagcattacgaat,
gctgacacaggacact, agagttccggttcagg, catttgcttgtcgtct, cattatgaatttggat,
tttgtcggagctctca, aacgtggcattttaaa, agcgagctggaagaac, agcaacacaaaaaatc,
cggtgatctcacatgg, ccttcaccccaaaggc, aagtatgatccacaag, ttatctcatacttcct,
gagcagctttgagacc, ttacgtgagtcacaca, caggaacagaagtgca, cccatctgtgagaccc,
tattgagaggcactga, gaaggtataattctaa, agggcttggctgtgac, tcttcatggcctcggt,
atccacaccaagaagg, ggcaaaagctttgtgt, cttaggcacaggaatc, atgaactggtgtaatg,
aactagtaggcaaccg, caattgggggggtgag, cttgctcccaaaccaa, tgaagccccgatagaa,
tgctcattcttagatg, aaacggggggtaaata, tttaaaaggacgctca, gaggtttttttgacaa,
ctcgcagttagggatc, aacataggctggacaa, agccaggttaagtggg, tcacataccgtttgct,
agaccccaaaatgtag, agttatgctcatggcc, atctgtgcggcctttc, tagagggctaaaatcg,
ggtgtaagctactcag, taccctttttttatgg, gcaaacagttggtgaa, ctctttagtggcaggc,
ccggcttagaggaagc, gattggttacacccgc, cttcccccacgaaaaa, cacccccccattttgc,
taaccgtgggcttgtg, atcaatcctatcatag, taaatacccggagaca, gcggttttttttggtt,
gctagccaagatctca, gagcgctgtgttcaga, caggcaacattatgct, ttacccgcacatcttt,
cgctgggctctctaca, acggcacggctggccc, ttcccggagctcctcg, atacgcgttcattagg,
gtcgcatgcctgtgat, ttaccccccttccatt, ataaagctggtcctta, cagacagtcagggtgt,
gagtcttaggtaggaa, gccagcaccttgtata, acatttttttgtcgtt, acataagtatgattct,
atcgtttttttccctt, ctttgccagggggaca, gggtactcctgtaaga, ctcctcctcgctctaa,
ggacattagcctaatg, caaatacgaacaagtt, gtatttagtttctgga, ttttccgcatcaatta,
attggatgagctgggc, tccgaaacctgaaaca, gggccatattctttcc, atgacggtttttttta,
tcctcgttgagcacgg, aggtcgaacatttttt, tgtgaggcttttaact, atcctaggtgaagcag,
acctggacacccaccg, tgttagagaaatgccc, cttgcgttcgcccaac, agtgagcaccgtgcat,
ccactctactgacatc, tggtcaaatacacacg, tgtatatagtcatgcg, gtacctgtgaccagtg,
ttagtagagtcaccac, ttacttttaggccagc, tcccggtactttgcgg, tcattcagataaggat,
gtccattaagtattac, gcatagccccccccaa, ggtgccttacttacag, acacccgctgaaggat,
ctaggttaaataacaa, aggtaggcaggtcaag, aacagtaaccataatg, gaggtgccggtgtgag,
aacaactccagattcg, atactgccagacgtat, ccccgtggataagaga, tagcgctcaatccctc,
tcttatctgaggcagc, agtatgttactatcct, tctgatagaacacgaa, cgtttttttttaccat,
gccgcggcccttggct, atcacaccttctgggc, tctagtagttgttagg, tacgataggctgattg,
cctcgccccggtggca, ttgaggcgattctctt, cggacgtagtggctgg, tgacctatccaagttt,
ctagggaaaaaaaacg, cccattgccttgttcg, gatttaaagcagatcg, ggcgtccgcctccatg,
caagataccaaagtcc, ctagtctgtgagtggt, tgggtatctatcccgt, ttaaaaatcgctttgt,
tgagggcttgtcacca, acagcgcccgccttag, cgctcaggccctcggt, gagcctttaacaaccc,
agagggtgatgggtat, atgacttaacgccaca, tgaggtccaaacaggc, agttgtttttgtaatg,
acgttaacaaacttct, ttcaaagactcccata, aaaagctgctgtccac, cctgtttttccgtcaa,
tggacatccgccgctg, aagatctcgtggcccg, gccgaaaaaaacacaa, ctcacccagactgatt,
gttatccccaatgttt, cccccgagtgacctcg, ctccccccccgctcca, acgtttgccttcaaac,
cagattttttttccgg, gctgatggaagtactg, gtaaaaaaagactatg, actatctattgactta,
tatgacattaatatgc, cccggagctcctcggc, gttttgtccggagaat, tgatataggcaggaga,
tttatattagggcaga, taagtgcctcttgttg, ctgggtcctgaatagg, ggggtccggctgggcc,
ccggttcacttgggct, tcgttgttgttaataa, gcggccgggaagagtc, aaagacaaaaaaagcg,
ccaaaaaaaatcggtc, atcgtgctgattccac, atctgagaagaactcc, ctgtcggctattggcc,
gaagagagtctcgaag, cccgccttttttttat, tattggccgacagaag, ctctaagtgcactcgg,
agtactggtgtgggga, gaggcggcagtagtgg, ctttcggtatgtatat, ggtactgtgttaacat,
ttcgccacttttttaa, ccctttggaacaccgc, acatggcatagtgtgt, ggaggctacggggacc,
gaaagaaaatggcgtg, cctgaagtcccccccc, gttccatctttccaca, atctatgattttttgg,
tcctatcagggccaaa, tctacattaaggtgtt, aaaaacgggggttcag, tccgtttgaaaaaata,
ggggctgactatgttt, cgccggctcctgcaat, ttggtcttctcttagt, gatcttatgggggggt,
atccagcactgtagaa, gtccggtgctgacgaa, ggcttagacgatggga, cagacgtcagtggaat,
aaatccgtggaatgtt, cgtgggtgccattgca, ccccgcttagcgcagc, aatcgtccatggaact,
tcatgttagactacca, gtcctcgtggctcatc, aaatgaggtgggcgtt, ggatgaattcacttgc,
tgaagaaggtaatacc, gtctgggaagcctcga, gctagagtggtcatta, ggtgcagtctgtgatc,
aatttcctagtaactc, ccgggggggcccgaag, tgaattggcaatagct, ttaccatctagagtaa,
taacgactgaattaat, acggtgatctcacatg, acgctaaaacttagag, tatagacgtaaggtag,
agcgcactccaccaca, gcattacagaatccct, tgtattacaagtaggg, ccacgaagtccaaatc,
agttactcgtaaggct, attccttaaccctaaa, tggaaaagctcctatg, tttgaactggtaaagg,
tctattagtttatatc, gggcaacgcaaagcaa, atcagcaaaaccctgt, gtgtgatgacatgact,
atgttacctgagtctt, tgtagttatagtgtcc, atggagcaggggtttc, aggagccctggaccaa,
gtgatcgacccacctg, actctaaaaaaaaggt, ttgtccccgagtctat, tgtgcacactgtgcaa,
caagcaccttgaggta, gaatgacttgtctagc, cccgaaaaaaaagtta, gcccctttttttgcta,
cctgggtaccatatta, gttccctcactgaaac, gggaggcctggatgcg, cccgatagaacatcca,
atataaaaaaaacgtt, gctccgcaggcatgcc, gtgtagatacttctct, tgtccagcagctatat,
ccgtgcggccgcccac, aacgtgaagacaaaca, tcgacctgccaggttt, ctaggctctgtcacaa,
tgatggacttcacagg, ttagtagggccaggtt, tacatgctgtcctcca, tttcacttccagaccc,
gctcgcggccttggaa, gtaaaaacctgggctt, taccacgtgcttctaa, agtccccactttcgtg,
ggtagcaatgacttta, acacatgccatatctg, ataaaaaaaacgttca, gccattcatgacatca,
ggcatcagttgcttac, ccatatgcactcagta, actaacaggcatttaa, cttcgtagatccagcc,
tgtggtcttatgtaat, ggctaaatcatgtgaa, acgtaggataagttcc, tatatgttgatctagg,
cttcccccccagatta, accagttccacgtgaa, agaggcgctcctgaca, tggaccgttttccctt,
gtgagcaagagtgtcc, taagaggcaagagctc, caagggtctgcgggta, ctgcttacagtagact,
acaatctacctatctt, tttgcatagatgactg, gcagcatacgtcagct, tcagcgccagcctcag,
tttgcaaattgcgcag, tctcatgctctggtta, ccggttcaggacaacc, atgtgatgttactgct,
taggcagcatggttaa, cgtaagctcccataag, cccttgagtcaaaata, ttccgcactcttctta,
actacttaagactggt, actcttgggaaaaacc, catgggaggccacgtt, actgacagttaaagag,
gatctaaccctgatgt, tatcccatctaatctt, tcatattgctctattg, aacatgtcattgggct,
acaatggcggctgggc, gatggtctgcctggtg, ctcgtcttttttttga, aaaacgggcatgcctg,
ttgcagtccggatggt, ttatatgaagccagct, aatgacttgcgaggcc, ctggagtgactcccct,
gaccactcctgtaagg, cctaacagtgtgcctc, cgttatccgctcgcct, tgaaagttcggaatat,
tgcattgtctaattgt, taggaagtaaccgcag, cccgaaattcatgcca, tgagttggctcttcat,
ctaatggttaaagtga, cttttccctgtagtgt, cttagtacccaagtcc, tgactccccgcattgc,
accatgcaggatcgtg, ctaatggacaggtgtc, aggggacccttagtgc, ctgtctcttagcctgc,
ccaagagcccccccca, gcgccttggtcaaacc, tgaggggatagaagat, tcttaatcaatgactg,
ccccccatatgctgta, cacgaataaatcaatt, ttgtggtgaactgtat, gatttcaccctttgga,
agcccgtccggtgctg, tctcgttgggtttgtt, tgactaactgatacaa, gttgcgtaaaaagaaa,
tcagcccccgtgccag, aacgtaacttagtagt, agccatattggaatca, tgtcaactgtgagggg,
gttcccaaccccccta, ttcggcctgtgattac, aggcaggggggggtgg, tggactcggggtgtct,
gaccaataagttggtc, cacgcccagggggggg, ggtaatctcatcccta, ttaaattagatactgg,
ataaaaacttcgctac, gtaaaaaagcacagca, gtgcaacactttaacc, taagaccatagaaacc,
gccattgttagatgtg, gcttagcgccgcgcag, aggctaacatggttaa, tttaggggcctaggcc,
taacgccccacatgtc, tggtcgcctctaatcc, ttaaaggcacagctct, ttaagcatgcccccca,
gaggacggcttcagtg, gggcaggatgatgtaa, tctgtgaggcccgggg, gtgcttttttcaatat,
gcgtgaggcggccatg, gtgaatacttaacctt, tccacatagacccaat, tggactgaatgtagat,
tggcattcggccagtc, ggtttaggggcctagg, caatgaatacttataa, tcaagccgctgtgtgg,
ttacaacagcacagag, ggagctaagcggtgag, taatttgttgaccact, gtccggtgagaactgg,
cgtcgaaggcaggtgt, cctggaaaactcgtaa, aatagtacaggtatgc, ctctgttgtctgccgc,
tttattccctacggta, aggcctttagagacga, tatttcgttaccttgt, gaggaaagccttagaa,
atgaggattaggagct, cctgatcaagtccggc, cttgtccttatatggc, aacattgtcctgtaag,
cagggtggtagtagcc, atacacttatttgtga, gattagttcactaata, ttcacaagcacccatg,
tccgccttactatttt, gagttaggtagactct, tcaaccagatactata, tcccagacgcctttgc,
catccgcttgcctcct, atcttattgtttaggc, atagatccttctctta, tgctatgggtggattc,
acagccccccataatc, agccttgttatgtcat, tcgcgtggtgacaggc, gtaaccaatgtgtaat,
cctaggaaaaaacggg, cagggttagaccccat, cttgattccccccccc, gcaagtctgtagaact,
atctcgtctgttttaa, gttagaccctgttgtt, aatggagagcgtcttc, actttttttatcagca,
tatgaatggcattact, ggcgcattgcgccggc, cagtggaggggggggt, cttatggttgatttcc,
ttctctggactcaagc, gaggcgccgttccccg, atagaggcagacacct, ggccgattggtccttt,
ctctgtttgtcactgg, ccccgtgtatcccggg, cttttacttacaacgt, gaagtaaccgcagtgg,
cggccgtctcaggctc, tctgggccactccgct, ctcccatccgttgcat, aacatttcagggtcat,
catagcgtaaaaaaat, ttagcccggttccctt, actatctgtgagttat, gtggtctagtctggaa,
agagataagcaagcgg, tgccatttattacgca, actggattattaggta, tctcttcgtcgggacg,
gggccagacagggctt, tctgaactgccattgc, tcacaaagagcttgcg, ccgtttataaagttga,
gcatgagagtggcgtg, acgttccacctcatcg, tgagagtggggggggc, ggttcttactaggcag,
tagcaagttcactagg, ctcaactattaaatag, tttagggactaagcta, ttacgaacaaaaaaaa,
ataccaaattagctaa, gacgtggcagggaagg, cctggggatgcattct, tctcttacatcccctt,
gaggttccaccctcac, gccaccagggtgatta, taaagaaatacgatga, tcccaatgagaaccat,
agtgaaatcaaccgct, ctatcaatttggccta, ataatacaacgcgccc, acgaacaaaaaaaagc,
ggtaacatttttttgc, tgaataggtcttcact, tgcctgaatctgcgtt, gcaatagatcacaata,
ctgggtagccttttga, gagggggggctcacta, caggcgtggcggccat, accgttgatcttgcga,
ctttgttcctggcttt, tcactgggaacgtcag, tatttacacactcctc, gatatccccagaacat,
ccacttgctaatccca, tgttcagtgatgtagc, gccccccgctatgcgg, ctggtaggactagggg,
gataactttggtggtg, agtagagaggttttac, ttcgagtctcctttat, tgtgcatttagggtga,
catgggttttttcatc, cgtaggcccccccgag, atgaataacattaaag, ccaactgggacattcg,
ggatcagcacggggca, aagactgattatgatt, ccccaaattcccgctt, gtcaaaaaaaagcgat,
ccgtttgttgccttag, ttgtagagaagtatac, tcccaatttcttagga, gctgggcaatttgctt,
cacacactgaagcgtg, aattagccgtaaatca, agttgtcccccctgct, ccatgcagtaactgtt,
gtggtatcctggcgtc, tttatagtcatcagtc, agttgttagggatctg, caaccatggaggtctt,
ataagggggggatgtt, cgatcacctgagtaag, tcgatgtttggctgtt, ttatgtagtcaggagg,
agtaaaagatgggtgg, gagacctcgtttataa, cagttttttttgtgac, cccctagttcacagat,
atgaggacaagctttc, cctgagggtatttaag, tagtctggtgttccct, tccagttgtacacatc,
gagcagcacctggttg, tagtgctgctgagtac, gggcccatagaatgga, ctttttgctcgcccgc,
cgtctgaggcttcacc, atactccaatgacttg, atgccacctaagtgca, tgacatgcttcggtgt,
cagaggttctagcgat, ttaaccaattgatgtt, atgcacgccgtagtcg, gcatcctggtggtgaa,
ttcttccacgtgttcc, cagtcagcaatgccgc, aactaggccgccgaca, tcaataactctaataa,
actccaagacaagacg, aactactggcctggtg, atctggtgagccacac, gcgaagtcaccttgag,
cagttctccgccctca, tacgaaaaaacatgcc, caggatatccttacca, gaccagcctcaaatta,
ttgagcagaaacgtga, gatgcactaaaggaat, cggcgagttaatacac, ccttatagaagcatcc,
tttgactcgaaaggag, gaatctgttattgtac, tggtggagtcaatgtc, cgatgtttggctgttt,
cagttccagtatttta, tatgcagatgaccctg, ccatggtggggggact, tgggactgaggcaccc,
gcacgttactagtctg, gcttttttttcagcac, gacaaacaccatctct, gccaaaaaaaagatcg,
ggcccctggacactta, ggaggggggggtgcct, tcccaatatgtgctcc, agtttggtcatagctt,
gtgcctaaaaaaaggc, tagcgctataactccg, ttatcccgggggggcc, gcctaccccccattac,
tgcactacaagatgga, taccgaaaaaaaagac, acggcttaaagctatt, tcaatagagtaacttc,
acatcactaccctttg, tgcagattagtgactg, gctcactgcacgtcca, ctaaaattcgccaggc,
gaactgcggaggccta, cacattctgcataggt, gaggcggtttacccca, ttactcttcgttctga,
aggtctgactgcggac, ggagtggtgtccatgc, ttttggcgagttatat, aactcggaaaaaaagt,
caaggtggagcacttg, tctatccagtagtgaa, ggtgcccctgagtacg, tactgctgcattattg,
atcctttctgtatggc, cacgtgctctcccatc, gaatatctagtacttt, aattttagggactatg,
catggaaaacggggct, tgagtgggggggggac, ttagaaacatccatga, gcccagtcagtgtcta,
cttttgcaccgcttgt, aggcccagcctccgtc, cttaagctatctacct, atggggggggggagag,
aagtaatgtgattgtc, ttactcccaagctatt, tatccttaccatcacg, cctcccgcctgtgctc,
gtacaccaattagact, tgttttttttgcaacg, cagcgttcggcctgtg, tcccccgcagcgtgac,
ggtgacacgagttaga, atctcgtggcccgagg, ggcaggaaatttgcgc, cacgtgtgcatcccac,
caggacaccctggata, ggagtgatagaaccaa, acgcactgagcccctc, cagcatcttcacgcaa,
atagagctgattggtg, atccgctctgcctggt, tacttgcaatgtttgc, tggccctacgatcatt,
tggcactttgtgggga, cccagttactcgtaag, atgtcttacagattgg, ttaccgcgttagctag,
gatgaggaatgcaaat, gggatgccctgtctga, cgaagctctcacgccc, tgccctaccccaacta,
ccgcggtgccttccac, ttattaggcctccctt, tgtagggggggacggg, gcaaggttggcctagg,
gcagagaaatactcgt, cgttgcgcacttccgg, aatggtcatgaaccta, caccctaaaaaattgt,
taccaactgcaactat, ttagcacctggttaga, ttctgatcccagtcgt, aagacgagggttccag,
tgtagccattgagtaa, cgtctttgctaaaata, gcacaatacgcccggc, catttttttgtcgtta,
catgcctcgggttgta, atccgatatgctctcc, agggtgtccgtgtctt, ttgattacctttttta,
ttacggaaaatattaa, attacataccgtggcc, ggcaatgcctcgatca, attcgggagacttagg,
atcccctaaaagaacc, gagacctccaaaggtg, tatatacttcgtatac, acaactgaaagcttaa,
ttggggacccttttcc, ccaaaaaaaggcgatg, ggttttttttgagtat, ggggggtatcatggag,
aggcaaaaaaaggcta, gtgtgtgcccccactc, ggctgtaggaagaaat, tgtttgtgtcacagtc,
acttttctaggccgag, tcaatgtcacttgtgg, taagtttcctttagtc, gaacatgagcccccga,
agtgggtagaatagac, gatggaaaaaaaagcg, ccagacacttgaaaca, ccatacatctaggtaa,
gacttaggcaattgca, cctctggaactactta, cccggtctcttcagtt, acagttagactcccat,
agcagccacgcaggtt, tccttctaactggcac, tcctataataggccta, tggcaatcttaccaaa,
tcaccactggcagagg, agatcttcccttagat, gcagtcttggtccttc, gcagcggcgggagagc,
ttactaaacgtagatt, atgggcctcggccttt, aacaggtcctttttag, tgagctcgggggcgaa,
cctttggcccggtggc, atgactctcggggttg, atccctttccctgaac, gtgtcaaactcgtggc,
tggttgtgatattcca, ctgctggggggtcaac, tacaatttaattccca, cctttggaacaccgcg,
gctggccccagtatat, tcatttggtgtagata, tctccattggcctatg, tctgacctgatacttg,
gtgcgcgggacttcca, cgttaaccacctcacc, ttcactcattaagctt, gtggaactgactgatg,
taagatgaagatccag, tacactcttgtggatt, ccatggacacttcgtc, tgtagtagtcacatac,
gatgtgttgcgttttt, ctctgtggtctagtac, tcacagttatgctgtt, tgacttgacggttcct,
ataggtccccccccct, ctaaaaggccgtgtgt, gacacgttcaaatgat, caaactctttcacgag,
ctaggtcgggagattg, gaggagttctcgcgtg, gacagcgtgtaacagt, gtgtctgagacactcg,
ggtctttaagctgcat, gggcaacagtgcatta, atgccctggtccttta, gggcagtctggatcct,
ctgtatcctctaaata, aagtgtaggtctgatt, aattactcctcatccc, tctatttctcgctggg,
ccctcctctgtcccgg, atgatgtaaatcaaac, tggctgtttaacaagt, aatgtcaggtgactac,
ctgctgataacaagtg, gtgagctttacccatg, gtatatgaatggggtt, aactcatgtcgctgac,
aaaccaccctaacatg, aattgtcaaagtacgg, gatgggaaaatcatgg, agtgtttgtcgctcaa,
atgaattggttagata, cgtttttgaaggtttg, tccgcgcagtctctgt, tgagcctcctcgcccg,
gcaagctacgccttct, ttgccaaacccaatgc, taccatggccttagtt, gtgctggagatgggta,
gggcttctcttggcac, taatgccggcagtgac, cctctggttcatgccg, aatggtctgacgctgc,
tgggggagacatgaat, gctgtgagtgttttaa, aggcagaatctaagct, aaataaaccagacgaa,
cggatcaggaatgtgt, aatactccatccatgt, ctctataaaaaaagtg, gtgtcggtggggggga,
gaaacgactgttctaa, ggaaagtcccagtgag, tgtcaatagctgaact, tcacgcctgtaagctt,
cacggattgatatggc, cccgataatttaatgt, accccctaagggggtg, ttccctttggaacacc,
ccgatatgctctccac, tttaaaacgaccgatg, gagtatgaattaagca, gtagcgaggcgcgcga,
aatgtgcgctagccaa, gctgcggcttagaagc, taggagagtctgaatg, ctctctaaaaaaaacg,
taaagtactctggggg, gtgacctcccacttgg, ttaggaaaaaaaacgc, gagtggtgtccatgcc,
ccactataatgggagt, tagggggggcaggcat, catgggcaacgcaaag, accttaaaaaaaacga,
gctgattccaaacatc, ggttctgacatttcca, aacgttatccttgttg, gacatatactcaccta,
tctcagaacgtaggag, gttgcttggaaactaa, acctggatcttataag, ccctatgttttttttg,
cagctttgcacaatat, tgaatactggtcagat, ccatccgatttgtcaa, agttaggtagactctg,
cgcgttcattaggcag, cctcgagggcaagaga, gacttgatactaaaga, gcatccctaattgctg,
ccaggcttcccgtggg, atccagacaactaggt, gaagggcgaactctag, gtcgcataaaaacagc,
tacgcgagcccaagta, cggggtgtctagacat, ctccccaattagcctc, cttagggaagttgagg,
ggctaccacttaaacc, gcaggcttagtgatgg, ccatgtatactccagc, ccagtattgaaatgtg,
agagagtctcgaagaa, tgttcctcgaaaactt, cccaagagcccccccc, ttatggcgtgcaggtg,
ggaggcctaaagtaca, tccatctgtgacgaat, ttgaaaacatgggtcc, ccatatggggacgtcg,
gatgagaggcctcaat, tccaaatggcctcctc, cacacctgtgtaatgt, gcatttgctgccttcc,
agcttagagcggttcc, ccgcatgggcttctgg, cgctgtgttgccgaga, ccctgctgggaacgga,
tcttaacaattctaaa, tgggggggggggggaa, atcgtgctgtaatgaa, aacagccaagcagacc,
cagttaaactgacact, ctttaatcctgggggt, gggactcggtttttat, caacagagtcggtttt,
aaacaagttatgctca, taccccccacctccgc, gcaggaagccaaggcg, tgaatctgcgttatac,
cggtagcgtgaagcct, taaaaagtgagagggt, ggatgggttgaggata, gcttgaacccgtggag,
aatgaagcctgattag, cacacgtaaaggggaa, ttactccttgttgtag, ctgtgttatcgaggat,
tctatatctatcagaa, acctaattctttacat, atcctgagcttacgaa, atagcactcgtgtact,
aattttttttgtctcg, cccgttgtcccttaga, cactcaccttaaagtt, gcccgtgccttcttat,
cctctaatgaccaaga, tgtttcggggggaggt, aggccgaaaaggaaac, gctcgcggcgcttgcg,
atgttcatactgtact, ctcccagattcgtcac, actcaccctgcttaat, tgtacgtggatattga,
ggatgcagctgaggat, aatcgtgtgctgtggg, tgcaacttgcctgatg, cttaaactattccagt,
tagacagcaacaggat, cccaccgcagtgtcca, ttttgaagctggacct, tggttattgaattcaa,
gaggatttatcttacg, aatggcctcccaggcg, acacattcccgaattc, tgggggggctttaaca,
atataagttgtcatta, tcgacccagcaaccca, cacccacggccactac, gtgcccccccccgate,
gcctcaaacagagtgg, ctgttcctaattgtgg, gctcgtggacaaaaaa, acctaataataaactt,
cggcacatcagaggat, aacaatgtgagtgaag, aatgatccactgtctt, ccgtgagattatatcc,
tcattaatgtcgcatc, gtgaaaatacaacgga, aggtctgaaccctact, aagacgggctcttagc,
gaaattcagcggtgcg, tagtccttagttcctt, gttggctccgtcctta, cgtgccggatccctga,
gcatgggagcaactag, attacaattgatcaaa, cccttggatcagaggc, gcccaccccactaaac,
ataaaaaaaatagtcg, agacctccacaattgg, gtgaccccactccttc, tgggggccatggtgca,
gtcacgtatgaagcag, atcaaagttaattcca, gccttttttttgaaca, atgtttcttctgaagc,
cggaatggggtaagtt, tcgattgaagaaaaca, caacacgtgcttgaga, tgagctttccctgata,
ttgcctgcgcaggact, gttcgtaagatctggc, cccggtaccttagcct, atgtcacctcaagatt,
cactcacgtgatggca, gaacgaaagggaaagg, agcatttacacggcca, tatccgcattttacca,
caacacgctgccaatg, cttttcaatgggagat, aatcgtgctgtaatga, ttatgcaaggcggaac,
cactttcattagtccg, tgctccccccttgttg, gtgtacccatagccaa, cagcctgaggttaagg,
atactgaagctcctaa, cgaggtatcaagccag, tccttataccttgttg, ttcatccccatagaca,
ttacttagttgtggta, tgatttttttcaacgc, tttgttatgttgagcc, ggagcctgagaccttt,
aggatactgtagtcat, taggcctggcaggcat, agcttccgcactcttc, ttgtgctgttgcactt,
tctcctgtccagctta, tgttgtcacatcatcc, gcaattaaaaaaagtc, ggacttgctgacggaa,
acctcgcccggccacg, aattaggccttaagac, attcattatagaaggt, tgccagtttttttatc,
acaggaacccccccca, ggctcttagcctcttc, cttcagcccccaactt, tgaataggcaatttct,
aaaaatgatacgctct, acaatagcccttgtta, agctgttttggtgaac, gcactttctctttcaa,
tagcctgacgtggttg, acgcttttttttaagc, acaagcgcccatttcc, gcctggacgagtgacc,
ccaaaaaagagtgcgc, ctaagaactcaattta, atacgtggtgcgtggc, ctcctatctgggtctg,
ttaatcggttgtcttc, gggaaggtataagcaa, ggttttcagcagaccc, gccttgctttacgggc,
acggaaaacttctcac, acctcctggttggcgc, aggctcggggctagag, agctcacagaccttag,
aggagtgccaaaccag, gcgcattaccgtgtga, gattttgccacccata, tccccctagggtcccc,
gtaaccaagttgaagg, cagggttgtcaacagg, tacgcagttctaccat, aagaaatagttccctc,
atattaaaaaaagcag, gttctatactgctgga, ttatgttctcgaccaa, gcctacaaaaaaaagc,
atactgtatagaggtg, gcttgggggggggaga, cggacttcgtgatctg, tacgctgagaaaagtc,
ctaggccagtttaccc, taattgtaatcacctg, cgtacctttaaaatca, aaagcgtagcgaggcg,
cctggccctatggccc, ccagtttagcccagat, ttctctattacggata, cgtgactctctcgact,
ggcgatgctttctttc, aaggaaggctgcactt, tatttatcggtaagta, aatgtgtgggggggac,
acatggtggcatcaga, atcacaggtctattca, cgtggctgcgcagtag, cgacttgcaatttttt,
tccttatcttacaggc, ggccccttctagcatg, atactctaggtccaag, aggctactggtgcact,
cagtttcaggtggata, aaagcgagcaagaggt, gactttcagagctctc, tcctcgcgatacatct,
acatgttgccgtagtc, gacaagcttccattaa, tatggccatgtttatg, gtcatttagtacaaat,
caagaattaagggatt, ggaatgaccattacaa, tattcccattaggcta, agactcaatgtgcttt,
gcattgtggcacgagc, aagtaaggctcggagg, aggaccccccccaccg, tacagtgagcctagaa,
aaaagatgacgagttc, tgatagcaccatcatc, gtagtgacggggtatt, gcgctttccagtcctt,
gaggggtgtgcatacc, ggtctatagcttaatg, ctagagtaattacctt, gccaaaatgggggacc,
catcaatgcaagatct, aagacaaagagagcgg, cagtccatttgaattg, ctctttgtgggaccga,
ctgaatatcctctcac, gtgccttccgggaggg, gacaaagatgtactaa, ggcaacagggctatca,
ggggcatttttttagc, actagcagttgatttg, ggcttccccgcttagc, agttagaatgagatcc,
agggccgcgttgcggc, cccagagtccggccac, acttcactccttagat, attagccagcttatcc,
gagtctcaccctccgc, ttcacttagagctacg, agggctgaaaaccgct, cgacctgccaggttta,
taatggtcaaaagcaa, atctttactcgcctca, cgaaaaaaacccccaa, tgtctcaccactccaa,
atacggttaaaccccg, agatgacctaagataa, aaggtagctccattgc, ccgaggccgtagggtc,
gagggggggggtgccg, cacacaatcataatct, ctccttaagttattat, cttcgggagataatta,
gcataaaatcgcagcc, gatgcagattcattga, tcaatgattatttggt, gtacattcttgaatgt,
ccaaatcccagctagg, aggacgaaaatgaggg, agaaccttgctaaatg, acttagatgacaggct,
gttggtactgtgccat, agctacgtggccccac, ccgagatggcatttct, gacatcctaggtgaag,
gttactcgtaaggctg, gactacaggggtattc, ggcgacagttagactg, cgtgctggccattcaa,
tctttggtgtgaacaa, tgcgatttggtggtgg, aaatgggggggactgg, tttgtctcgccactgc,
tctcagaggttcatgt, cccatatatcaggcta, tcggggggctgacgcc, taaccttcccccccca,
ccacaagctagcctca, ctacccagaggtgctt, gggtgatttatgcctt, taatggaatccctaga,
ggccagtgcttgcgca, attagtctattgcaga, ttcaaagagcacgggg, tgtgttgggagactgc,
ggcttgctcacggtgc, actgtataggcactgc, caaaaagaacggacag, gaggcctaggggcctc,
ttgtgatattccagtc, gtctaaatttcttggt, ccctaaatctcaagag, gtaaaaagtcactcag,
tggccccagtatatca, ggctatgaatagagca, agggcccctcggagct, atatcctatctcatta,
ttagttgtgagcccag, gaattacaggcttccg, tggctcagcgtatccc, gcatagtgccagtctc,
aatgtgagtgaagtgc, gtaattttttcggtgg, cgtttttcagtttatg, acgcatatactttgtg,
tgggggggggagaaga, gcttacatgggtcatc, tgtccataagacctta, gtaaccaacccaaatt,
tacctgggaagcttag, acagcatagtcgacag, tgccggaacagttatc, gagctatagagagcct,
gagctgtgcaaagtcg, tgaccacagctagtta, aacttcataaggtctt, ggatggggcaccacga,
ttgcccagacaacagg, acggttttaaatttgt, cactgcattgccggat, aagtcatgggggggag,
cattgacccccccaaa, cctacgcccacacgtt, caaggctttttttgcc, tcagctcgtggacaaa,
ggtgtgtgatattgtc, gcacacagccagacaa, aaactctaactttcac, ctacttagtgccttat,
tataagcatatagcac, atgcagcaacctttgc, caggaccccctaaggg, ttgttctaggagtgac,
tttggcagcggccact, ttaatgcccccctttt, tacttaggctgttgat, agtatatcaggtcacc,
aatgttcctccagcac, ttgaccatttggggag, ttcttactgctagctt, gtcattaagagtgata,
cgtgttgtgcagctgg, aaaccacagtgttgac, acggtgtgtatatatg, tactgagggctgagac,
gttgattacaacctgt, actggtgcgttatgaa, cctccctggctttatg, ataaagtagtgggtcc,
aagcatgttccaaatt, acgggtgcgagtggtg, ggagtgctccactcct, caatccactgtggctg,
gccagcctgcacgagg, tggctgaccacggcta, atacactgtaggtagt, ttaccgtaagctcggc,
ctgcagtaaaactgca, tattgtgctaagatcg, gtcaccgtttttttcc, aataataaacgcagga,
caatcggtgatttgtc, tccatctatgtagttg, atgttccgccacaccc, agccgctaaaaaaatc,
catactttggtgtgga, aggtatacttaatgtg, tccacttgtacgatct, agccagtcattctgtg,
tgttattttgttcccc, ggtcgcagttagccca, atattcatgctgtcgc, agcgctagcgctataa,
attacccacgatgaac, acggctacggatgacc, actctgtggtcacgaa, tgcctgaaggggagat,
gaaattatcggaagca, gcgctccgcgcccacg, actgattgattatatc, atagtagattcaagcg,
atctcgaatggtaact, ataacaattatctgcc, gcagcactgcagtacg, ggcatagctttagtca,
cagctaaaaaaaccta, gcttaggcacaggaat, tgctgatggcgcgatc, ccaagttctgactgcc,
aaccccccacgcacct, tacaatctcccctaag, ccaggggggggccagt, gccgtagtccgggtgc,
atctaaaaaaaggctg, tcctcttgcaaacact, tggggggggtaggagc, aagaatgtcgcaacct,
catttaagcctagtag, catatatcttacataa, gttgaactctgggttg, attacccttggacatt,
ggtggagagccggggg, aatataacgaatagta, agctaaagagtcacgt, cgcgcgagaggaccaa,
caggagataagtgtct, cagatttccggtgctt, agttaccctgatcttt, taaaccccatttgtac,
ctctgcccgatcgcct, gttcttatgtattaga, aaacaggtagttgatc, ttttttggctttgcgt,
gtaaagctgaggtgaa, ctggcatgggccgggg, gcactgtggggggggg, tctgagcttattcaat,
tgataatgtggaccaa, cctcatgatccgtgca, ggtgcctgctgacagc, gcaatgcctcgatcag,
ggctggaactgccatg, tggcgaaacccttctg, gggcggtccttccggg, acttataaatagaagg,
tccttccgcaaacata, cctcccctgatcagga, ccgggcgcattgcgcc, gcaaaaaaacgtttgt,
agcaaaaaaacagtgc, ctaggtctcacgctga, agatgcagtagcacgc, tacatgggggggataa,
tttcgggtgttttttt, tcccagactttgagtg, atcagcacggggcact, acttatgagggcctat,
ttccagtttgcttagc, cctacccccccccagc, gtataagtatgcaaca, gcaattgaatgttaag,
aagggccaatggccca, cattgtcaagtattcc, tatgccattgtggacg, aaaaaacgttagcact,
aatctatctatgacac, aaatatgtgagggggg, ggcatattttggacaa, agtacttactctcacc,
cttatccactatagtg, cccatccggagcagcg, tccctaagtgtttatc, atctgtccaataaaac,
aacttttggaaccatc, gctttgagtgtgtaga, gctggacacagttagc, ccaaagtaccatttgc,
gctcctgagcatcttg, tcaacatccccgaaca, gcaattttcgtgacat, gagaaggctacatctt,
ggatatattctgtgga, agcttctgaagtggag, tgcccccccccgatct, ttctcccctgtgttaa,
aggagtgttcagtagg, caaactattgttactg, gtgatgatgtaaatga, ccgtgatataattgca,
tcctgcactcgcggca, tgtaaggttcaggaaa, tggccttagacggtga, ttcaaccgttgatctt,
cgcttcaaaacatcta, cgccgctcagcattcc, aagggttagaccctgt, taattgccattgccag,
gaccctgcaggttctg, ttgccggaaatctaag, tgtcggctattggccg, gtcattacagaaggta,
cccgtggcatggcaga, tcacttatctgacagg, acctgtgggttcaagt, ctctgtgacggcatcc,
ccccagaatacacaat, cctttcatcagacacg, tctgttcccctgcatc, ggagggggggctcact,
gagtgataagtctatg, taatattcccacctaa, tactaactgggggggt, ccttgagtctacatca,
gtgcccgggccccatg, ccccgaactgaaacta, attggaccttgtccac, gtttagcatagggaat,
cattcaccagtagtcc, cccttgggattaacct, tgttatatggtggcag, tggaggaggataggct,
atacctttagcataga, cactatagcctcactt, ttcaggcataagcctg, gtcacctaggagatta,
ttctgataagcatcac, agagttcctatgtagg, tgcctgccccccccgg, ttcttggataaccctt,
ccatgagatgaacgtg, acgctcggaggcacaa, aaagtatgccttctag, taactaattctggcac,
tcttggcgacagaggg, gcgaagaaattcagcg, cccaagtggggggggg, cttcaccccaaaggcc,
tatcagaaaggggaca, tgcagagctgcttagt, taaatctggaagcctc, tactaatcgacttttt,
tttttaactccgtctc, gtagaccccccccatg, ggatctccttagttca, agaggacagacgtctg,
cttaccaatcacttgc, gaccttagtcctatag, tgatagaacacgaagg, tgaattatgcccctgt,
cacttagagctacgct, tgagaagctattcatg, accccacaaccttccc, ttataaaaaaaaccac,
aaacctaataatttag, ggccagcccccccagg, tagctgctgattatgt, cttaggcagcactgct,
ccaattacggacacaa, gccggggggtagcttt, cccagcgggcagtgga, aggcacttcttgtatg,
aggttagaggatcaat, cccacgcctgggatgc, agctaagcggtgagga, ggttccgccccccgcc,
gattataggcgtgttc, tcacatgggcacctgt, tggtggcagtcgccag, ctataacttactctgg,
tgcaaccccaaatttg, aggcctattatgatct, ctcccccccatatttt, gtagggtcattttttg,
ggatatgaggcatccg, gggtgcatcgtgctga, aaggaacagcatctta, ggggtttgaccgcgtt,
gcttacctgagaacat, gacatgcagcttaggg, gattagccttcagtaa, tcaatatactcgaatc,
gcccatagaatggagc, gttagtgtttgttaga, ggaagctttttgacca, aatacggtagttataa,
acacttatgcatggtt, caattttgtcaccgaa, tcttgttgaataacat, ttttagcacatgcatt,
tacttccctacagacg, atgagaattctaagga, agtgcatacacctggg, tgcagatagatgctaa,
atgtcgtttttttggg, gagtggccaatcaact, gtgccgatagttgtta, aaggtaatcttacctc,
acagctcgcttctgtc, cgctcagcattccagc, agggtcacaatctagg, agctgcttcgggagct,
gcttttttttatctag, ggaaaagatcacagct, gtgtattcctactcat, atcgtgtagaacgtag,
ccatcatccgatatgc, atacagagggatgttc, ttcacatataaggaca, agcttaattgggatgg,
tcggtttcggctgaag, gctacctccagtgtgc, gggtagttgcttcact, ggtcctgaataggaca,
gtatcgtggggggtgg, gctttttttgggctag, gaccttcgggcagcaa, aattttgtagtgcagt,
gaactagtaggcaacc, ttcaccagcgcttcat, gcaaaaaaaggatgcc, agtgtagtaattcaaa,
aaaaggctagtcaact, taccacagtgctatct, taaccacatttggtgg, aaatagttaactacag,
ctgatagtgcagccac, ctgaagagttccggtt, tttgcggtgttttctg, ccacgagggatagtgc,
tttttttcgaattatg, atatgtcgaaaactga, tctaactcgtcaagtg, ttttcggtggaagagg,
ttccccccccatctct, gaacatgttgccgtag, gcgttcattaggcagg, tattagtagattacac,
ccacagtcattgcggc, gtactaaaaaaacccg, cttacccagctcgatt, ctcccaggtcttacag,
gtgtctactgtagtta, cctacgcccacggatt, gcatcagctgctcgta, cgctaaaaaaaatggt,
ctgtggcaccgaggct, gcttttgattatacct, cctctactattctaca, gtctctgcggcaagca,
acgaagcagttattga, ttggaaactaagaacc, gtatcttgacactcta, aggtaaaaaaaaaggg,
gaccctaaggctgcgg, actgtttatggctcta, ttttgatctggactta, ctaaggtgatccacgt,
cgccctgataatggag, atcaagccactaaaca, aatcttcttattatta, taagttatgtcaaaca,
tttatagcttggttaa, atggggggggcacttg, ggtgttgtagtctaga, agaacgtaggagagtg,
cgggggcgaaaaatca, gtaaacctgggaggct, cacctccctctgcgga, caaaaatgggcgctgt,
gaggaggccggcgagt, actctagctactgtag, ttcctttgatggggta, cacagctgtttgtttg,
gtgtaggtctgattta, ttagatcactaatgga, gctcacgtctgcaagg, ctcagtgtttgtcgct,
catctcagcggctctc, gatgacatgcttcggt, accctattagggtaaa, tacattagttacttag,
tccgaaaaaaaaggcc, aacaaaagaggggtgg, cctggcaaaactgaat, ggagccgttggctccg,
gtagactagttctata, agcattgtggtgcagc, acgggaaaataagatt, ttagaacacgcggtgt,
ctttgacgagcttaga, ttttgagctccaaggt, tcgatttggctgggca, tgaaaaaaaactaacg,
tatgggtaaaaaaagt, gcaccttgtgccagct, gaatttataggtctta, gcttgtttgcctccaa,
gcagtagaacttcttg, ttatgtcccacgttcc, ttgtgcagtctgaatt, atgcttgtgacacttt,
caattgttaaaaacgt, agcaggtgtcgcttaa, cacgcctgtaagctta, aaaccagcaaattttg,
tttatgccataagggc, catggcatgctccatt, tgcagagcttacatgg, cattactaatttccac,
gatgtgctgcctgaat, catcaggtggccctgt, ccatcactgacaggat, ttcccgtctgagaagt,
acaggcccgatttttt, ggcttgcgccaactgc, cgtgtggcttaaccta, gctttttttaagtact,
caacctccgcatggtg, gtgtctatccaagggt, tttgggaaggcactta, aggtgtggtgtatcac,
gggggggggtatcatg, cacacggaatccctac, aactcgtgttatttgc, cgggttcaaggagttt,
cattgttcggatgatg, tcccagtttagccttt, agtcccagtctaactg, tgttacctcattacta,
actagcctgcacatgt, gagttgatgaattgta, cccacataggcccttg, acccgccccccgtcaa,
ggaaaaggtcccacca, ggataagacctttcta, ccaccaccgtgtctct, tgcctattcttgccag,
gaaactattgcgattg, cttatagatatcagtg, cggctatgccggccag, atctccaggtttgaag,
caggagtgcagcgtag, agcatgagcaacgccg, cgactccatgtcttac, cccatagagggggaaa,
gtcagcccccgtgcca, aaggagcttgcaaccg, ggatgttcttagggat, ccccccgagtgacctc,
accgtgcggccgccca, cccaccaacgattcac, aattcaccttcttcat, aggccgtagggtcatt,
cataccctaaaaagat, ttgatcaaaaaaagcg, ggaggccagaatcctt, aggggggcatttatca,
tcagtggtgcatacac, attgttaaggacacta, aaggtgaactgtctaa, agcaacttatagctag,
aaacacataggtcaga, atcaccgtaccctttt, gggagtatttgaacat, atcaacgacttgtgac,
ttcccccatctgatct, attttggtgttcgtga, tccttcagtaagatgg, aaacgttttaaagtag,
ctccgttccaactgat, ctaagacccaaattac, cgttagcccgaggcac, atcaagtcctccccac,
gttggtgcataagaaa, attaatatatcttacg, gactaatagtgaaagt, gcgtgtggaacgtccg,
cataggtggctgtatc, ccaagggcagataacc, gcagcagctaacccct, tgctttaagcctcgct,
tacaattatggatgtc, tgtagtcagccctatc, gaagcctttcttatgt, aacaccctggcacttc,
taacccccccatgcta, ctgaagtggtcttctc, ctccttctatacttat, actggtgaagtcctgt,
cactcaggggccgcgc, cgcacaggcaacatca, gtatgacttaaatccc, cgtgagattatatccc,
tgcaaaaaaaagctgc, gccctagagtgggata, tacctactacgtcctt, cttaggcctgaacatc,
tataaagaactaccac, acattttgggtagtgc, gggaggccaaaagccg, gatccatttgccatta,
ccaaatagagtacctg, ctcccctacttccccc, ttcttccgtgactctc, toggctattggccgac,
gaccctgatgcccgtc, gaacagtaattagctt, gtcccaacacatttgg, ggaaaactccatcagg,
ttaactttatgggtgt, taagccaaaagcaagc, cagcacttagaggcat, atattacactcccatt,
acctcccggatgggtc, acaattgtattgtgac, cacagaacaccttagc, gcacaatgtatgccct,
ctaagccctcgctccc, ccctgtgtgacctgtg, cacctgcatgcggatg, gcacgtagaaatttgg,
cttattatcctgtcag, cattagttgttctact, ttgaggctcactagtt, cgaggtgggcccttca,
acagaactggggggga, acttatgactctcggg, agactgtgcacttcgt, aagggtgacttcctta,
tggttttggggggggt, ccgtcgtcctcgagat, ttagagatgacagagc, ggtggaatgggtgtcg,
tacccatcctgtttcg, cggtgcctgtcctctg, gggaatctataagaaa, taaggaagcttcttag,
gagatattcctgctct, tcagaactgaaaacgg, acactaagtgacacag, cgaaaaaaaaacacag,
ctacattttggcgagt, gttcctaacagttttc, gaggccgcgtagaact, tgaggtaaaaaaacta,
ctgattacatgagaac, ggcgctgagcgccgat, gtcaagactcatgtgt, aggacagtctaagagc,
ataagcaggatattca, gtaaagattggtatat, tccgcttcttcttaga, gattgtgtggcaacaa,
aagggtaatggctttg, caccacatctaggtta, ctcatcacaacccagc, ttcttccaccggccaa,
atgtattcagagaggc, gaccccaggtaagtaa, agacgatgcagcggat, atactttgaaatccgc,
aacctgaaagttcgga, tcttgctgagcaaagt, tcataaaaacgagatc, gtgcatgggtgatgca,
ggccattatgttatgg, atgctccaaatgtcat, ccttaatagtgtacat, cagctctagttggttg,
ttataaattctctatc, agttgacgtgcatatc, ggtccccatagagggg, tcgtgtgctgtgggag,
gacatttagagggcta, ggcaggatggaataaa, gtgtttgtttaggtcc, atgcctatggtggtct,
aatgtgcgcgggcgcc, aactgtacttatacta, acaaaaaaaagggcgg, tgtgttacccgcacat,
tttaggtaggtggaac, ctttttttgctatagg, accgctgaaaatgtca, agtatcagctttgtac,
ctggaggggggtgtaa, gtagactagaaaagag, tctgaagtcttactac, agtggtgggcggtgct,
ttaacgaaaaaaaaca, atcataagtcggctgg, tttggcgagttatata, actatactaaagattg,
cttccgcgcagtctct, tggaacatggcatgaa, ggctccaataggggga, tccatgcccccgaaaa,
actcgggaggattagg, tagaatggagctggtg, ctgtaactgcctgacc, tttttaacgtgccggt,
gatccatgtccatgga, cggctggggggagaat, ggaggtgatcgccctc, ccacgtgtctgtgagc,
gcactttgcttagcta, aagtgccttgaatgtc, tggctgtaggggggtg, ggtgggaggtggctta,
ctgaaagggcaaatct, tttgtctgtgctgcct, aagtgtgcccccccaa, acccacaactccacgt,
ttagtgcctggaggag, cagattcctaagatta, gtacgaaaaaacatgc, tccgttttttttagct,
atacatacaacaagag, acattgacgttttttt, tcactatcattaagtt, gacttgtaagctcctg,
cgcggtccccgtctgt, gcctcatgattcgact, ctggctaacagagtta, gagttggttgttagag,
tctaaaaaaaatgtcg, ctttcccctacgtcat, tgccaaagctcttagc, caccgcaggggggggc,
gaacaggctggcgctg, tcgcgcacggtgctct, gctgggaatttcgtgg, ggggtaggggggggga,
gcagagtaagttgtgg, tgacgttatttgtaaa, tcaacttattttcgtt, aatgttttttttcgtc,
gttcagtcttacccta, ggctgttgcatatcag, acgaaactgagttttg, agaatacttgtaaccc,
agtctgaggcggggca, ttattcaggccaacat, gagaaactagggaacg, cactgcggcttagcgc,
ttgcattaagacccta, tgcggaggaaaagcgt, ttcgtaagatggaact, gtagcctactcctttc,
gaatgtgtggggggga, gagttagactccgtcc, ttagaatacttagctg, caagagagaattcgtg,
tacgaaaaaaaccccc, gcgatatatatataca, agaataaaaaagcgac, cctaggggaggctctc,
agaacgttcagtgttt, agccgtaaatcacttg, attggtcgtatatata, gtgatcgccctcagag,
gcagggcattggagaa, aaaaattagccccccc, caacggaatttggggg, ttttagatgactacac,
tcattaaacatcttcg, tagcatatggaatgat, gagatctggtacataa, aggtgaatcatactac,
gcagcccagtattcct, gtgtgcccccactect, acaatgtgagccatgg, tcacctatggaagatg,
ggctgggactccatgc, ggctggtgtgccgagc, ccgcgcgagaggacca, gagcaccactcttaga,
ggcaagagagaattcg, cttcgggcagcaatac, acaatgtatgccctat, ctgttttttagagacg,
ggttagaccaacagat, cgcttttttttggcgc, ttcccattgctgcggt, aactttacgatggaaa,
ctgcgatgcatcttct, ttagaccattacttta, gtctaagacaagctac, tcaatctggggggggg,
attatccttgaacctt, ctcaaactctttcacg, ctaacccccactttaa, cctttatccagataac,
gtatgcgcatccctag, ggcgctgtggggctcc, gcagagctcggtggct, aagtcagcgtaaaaga,
gcggtggtgtgttcct, gctatctattcggttc, gatactggtttgttat, agtaagcagtgcacta,
ctaccaacttagcctc, taaggcgggtggctct, ggttaaaccccaaagg, ctagacttgaacaaac,
agcgttttttttccct, tgcgcgcggggctgtc, cgtgttttttttattg, tccggtctgtcattct,
cccacaataccaagcg, ttaacccacactgtgc, agcatgttttaagctc, ggagactgtatgtttg,
ttctcagcaatacggt, actagtgatctgggtt, agccgactctaacatt, ctgcttacatgggtca,
acaataggacttagaa, cctgttgtttatgcaa, ggacgagtgacctgtg, tgatcctcttacctaa,
catacaggctgaagca, aatgttaagggtatcc, aattcgggccattctg, cgagccaagtatggga,
aattgggtaaagcacg, tataaattgtctctac, gaatagcttttttagg, atcgaagccggacctt,
gagcctttttttaccc, gttagaagctgatgat, tctatgcaaccttcgg, tagcgctcccccccat,
aactgggcaggggggg, gaatgtgccaacacgc, cggggcatttttttga, cacgccccccatggac,
gacttgagggtcgcat, tactgatgcttgcgag, ataataaatacacggc, cagcttacccacatgc,
aggcaaaaaaaagcta, tttgttgcttcgaaat, aaagactaatgaacgt, gaggggaggctacggg,
actccggagaggggct, gggataataactatcg, aaaaacaaaaggcggt, taggggggttaggggt,
atacaatcgagcaaat, gttcatacaaccacta, ttattcccccccatta, gggcgctttccagtcc,
aatctccattctgaca, ctggttgctcacaatg, gtgtgtaagataaatg, ttttaggggggggtag,
ttgtgggactgatctt, agtttggatctaggct, ctcacatggggactgc, ccagagcaatgccgcc,
caaatgagagctcaac, gtcacccccccagtaa, tgttgttaatccttag, aactgttcccaaaaga,
taaccgccccggtctc, cttaactgttcctgcc, ggaagcgagccacttg, cggaggggttgggata,
actatgggcgcccggc, agtaccttggtaggcc, agcataagacacgtgc, atatggaaagcaaccc,
actggtgtgagaacca, agctgtgatgttggac, gtgtcctattattcta, catccgatatgctctc,
gcctttactgggttat, tattgagttagggtgc, attactcaatgtacaa, acgccctgtccgggag,
tttgagcgattttttc, atatccttttgttggt, gtgtttacatcagagt, cggttttagaagtacc,
agacgaaagtgagtat, gcgctattttttttcc, acctcacggggagaag, tcctgtccattagcat,
ccccctccccatgtag, ccatggcgatgaggat, atttgttagtgacatg, tgcccccccccaggag,
ccttgagcatctcccg, tttaaacggtgaaata, gatgttatgttaactg, ctctaggtctcatgaa,
gtcatagatggtcttc, gctcgtctggagtctg, acacatatttagccag, tcaccagcgcttcatg,
agtaaattatgacagc, gttcattaacagggca, ataccagctacttatg, gtcttaggccaggggc,
cccctgaatctgtggc, tcctaggtggaggcct, tgtctcgtcctcccga, cagtgccagtgcgact,
cttgaccttgattaga, ggcagagcctgacgct, ctgtatcccgaacact, tggtccctatgctctg,
gacccattatccagat, gcttaaattgactgac, cgtgcatcttgcgcga, ttagattactactatt,
cctagagtcctgataa, cgtatttcaacattag, gggctgtgccagcgtg, ccccgctaggcaccgc,
tgggcgttttttttta, ctcaacagagttaaac, cgtgcatgatattact, agcactctgcgtcctg,
tccggaacacaggggt, tcctgcttttaaactc, agtttcggagatggtg, tagatgctcaattcac,
caaaatctccttcagg, ccagcaatatcatgac, accgtgagattatatc, cgactgttggacgggg,
gctcaagcaaggaacg, tatcccataatcatca, ttttggctggggcgac, tttaattggggggggt,
aaaatggtcccccccg, ggagggtggtcacaga, ctctgattaagaattg, cagtgcatcatcaata,
gactcaacagaaacct, gcctgattcctctgag, tgcttcccgaatgctt, tataactgtcagaatt,
ctgctataaaaaaagc, ctactcacttttgaga, gattttttttgagtcc, atccagaggtgaagcc,
acagtttagttgtatg, tcagtttgactggcaa, ggagtcttactgattg, ccaatgactacttcat,
ctaaccagtgcaccgg, actgtgatccccacat, tgaagcagtgcgaggc, actggcgaccctaagg,
atgggggggttagagt, gagtaagagtccatta, tctgctttatcttagg, ctaaattcctggtgga,
cgcagaataatttttc, tctcctcactcggcgg, ttacataggctggatt, gcttaggcgggtgtat,
ccccataaataatttc, agcttgggtacactta, aagcccgggggtcccc, acatagtttcttgtac,
agcagggtcttagcca, attgttacctgtttaa, gaattgtaatgatatt, gcaaaattggtgagat,
gcagtcctattagcca, gtaagactagagtttt, tctcctcttttcgtca, ggtacaccacgggata,
ctcggggtgtctagac, acatttccatacctaa, gatgcactcagcttag, ctatgctaagacatat,
gggttccccagacgga, tagtagtaagcagtgc, ttggggggggtaggag, acagctaggcggggct,
acgttatccttgttga, tcttaaactcagcctg, gcgcgcctccacacct, tgttcatctaaacatc,
caagccagccacatac, agttagactctatcac, actcagcttagagcta, gagagcgagctggaag,
ccctaattatgcagga, gaaacctgttagggag, acggtagttttttggc, cgccttttttttatct,
caactaagctgtcaca, tgacctttctatcatc, tcagcacttgtctgat, ctagatggtgggtccg,
tacacagtttgcgata, tagacattccggacgg, agccaagttgggtgaa, acgaaaagtacacatt,
tgtcctctgaacagta, ttcatccaacggatgt, tactaacccagttgaa, gacaaacctcacctct,
caagacaaccctccag, tttcatggacagcgtg, gtatcatactaagggt, aaaatacgaggtaaat,
gtggggaaccactagg, ttacacatgcctccct, aggtgtgatcccctcg, ttctcagtgtttgtcg,
gcatgtagcttgtgaa, agaccttaagttaatg, gttagactggcccttc, tgaagaagcttaggcc,
cagcgtgtgtgtgccc, acgagcttcgctgtta, tggcccagagccgccg, gttggctaaggatagt,
atggtactctgagctt, ccaaggtcccttagcc, ccgggcgaagctcagg, accgtgagcattggga,
gctagggtatatctgt, ccttcgcattgacctt, acggataatttgccac, accggggcccatagaa,
acagatctgaggccat, ggtaccccaaagttgt, cgtctttttctggtaa, ccccgagccttgcgag,
ctgccaccgtggtggc, ccttgcagctgcggtc, aatcgcagcctcaaga, gattactaacacagtt,
cagcttagagctatca, actgtatcggttttag, cacctoggaaacgcgg, aattatgcactgccca,
atgtgctggcaccttt, acaattaaatctcacg, cataacctggatctta, cgacggagttagactc,
acgttatataaactct, agtatgttccaagggc, ccggtgggcgaaggcc, ataacaaactgttctg,
tggaagcgagccactt, cgcatgacgaggtcct, gatgctcttaacaggg, acaagcaaatatcata,
tctgtgtcagggtgca, atccatggcgctgcaa, cttcctgagcccgtcc, tcaagactcatgtgtt,
tgtgaggggggtacta, gtccccatgaagctac, ttagacaccgcgcctg, cgcttttttttcagca,
cgatgcagcggatcct, gggggttttcaacctc, aaatagctcaatttgt, tgtgtgcgttgcaaat,
cgtctgtctcttggtg, ctccagacgctccgcc, ctgcatgtattactca, tcactggtgtgtaaga,
gcggccttggaacgag, tttaactgttagaacc, tttatgttgtaccacg, agctgttctctatacc,
tttaggggtatggggc, aattgggggggcaatg, tgcaaattgcgcagtc, tccatataaaacagtg,
tgtgtctcttgagtag, ccacacgagaagagag, atcgtagtgtttctca, ttgccgtagtccgggt,
cccgaaaaaaagcata, gcggaaaaaaaatcag, atacaagcggattctc, tccttgttcctggttg,
gaggattacccatggt, tatatagacacgagag, ctggctcagcgtatcc, tttttagcgatgtagt,
attcactgtcagcgta, tagaccgtcagggaga, ttctaatcggccattt, tgcctgcgccccccct,
gtgttgaaggtgtgat, toccaggacgctccca, cctgatgtcagaagat, tctgaaattagcaacc,
aactcacttagagtaa, gtaaaggcttgtgtcc, ctgaaattatacaggg, ctgcgtagcccggcta,
tcacccccccaactgg, ggcggagaaccttgtt, ttgatagcctttagcc, cgttatgcctgagcct,
gaaatcataagtcggc, gagattggactcagag, tattgcctcgtccatt, gatccccgtgtatccc,
ccacttcgccccctta, tccaaagaccaaattg, gccggtttgtactgat, gtgatgctgcgtaatt,
ccgctgaaggattggc, cagtatgctgaccact, tacatgcatgtaaaga, atgctagccagaatgt,
gctaatgtctcatata, tagacacacttgggcc, caacggactgtaaatc, tcctctaagctagcta,
tcagtcaataaaacta, ctgttgtaagacacct, gattatgagctcaggg, ggcattggggaagcgt,
gctttgatattcaaag, gttggggggggatgct, actccccccacttgtt, ctactttaatgctgct,
tgccttagcactgccc, gcaatacacggccctg, tttatgttccagtacg, tatogttgagaaaatt,
atgaatcttttttgag, ggcttcaatgttagga, aagtaaaggaacttga, catcagaacgccctgg,
tgctctcacagctcaa, cttagtaatctgaaca, aacacctactccttag, gtttagcccagatgcc,
cccttccaggagcgcg, tcctgggggagtaggt, tatagtctgataacaa, gttaaactcaaggtta,
attcctgcctggatac, accattttttccatcg, aagcccaggtaaatct, ctactgtgtttatgtg,
aaaggcttgtgctggc, agtaccaagattttaa, gtgtgttaaactgaaa, cgtataaattataaag,
ttataggctcctagga, ctgagtattaactgat, ctaccagtatctcggc, cgggggggggcattca,
ttcgagggctcagagc, tagttgggggggggag, ttagaacatcttattg, tttgggatcactaggt,
tgtttccatccaatca, taatgggggggtcagc, accatccatgcccccg, ttaaccettacttcta,
gtggggggtgtatact, tgatttacccaactcg, aagataactttcagtc, tgagacagagcgagtc,
ctattataagctctaa, ggtttccctttctaat, agtagagtgtttcccc, ttgaaaaacatcgttt,
ctgagctctcgtcttc, tggcatttgtatttcg, attttagcattaagcc, tcaattctggtagctt,
gggggttcagattcct, ctgacaatttgcccac, caagccttgatggtct, gggtgcctttcttgta,
aagaggttgttaaggg, tgagccggcaggtgta, cgtgagccacaatgtg, tgggatttcggtgtgg,
gtactcgcagttaggg, cacccaacaacgaaag, taatttattacgctga, agcaattctccccaat,
taaattgtggtctaca, caagactgctgttccc, gcccttcacagtgggt, aatttttagggggaac,
ttttataacgttgata, ttacactatgagaact, acctccttcgctggca, accattactaatttcc,
gtcgttgaatttacct, gcagtagctggtaatg, gacgagtgacctgtga, aacagacgtgatccca,
cattctctgtggacat, caaacaaagaacttgg, tacgcccaaaactttt, ctgacagacccccttg,
aattggatggttctat, cagtaacaaggttcct, ttccccagcttattaa, taaggagatacccatg,
ttgctctcactataac, gtccccgagtctattt, actcgtcaagtggctt, aaccatacactgttgt,
ttaatggggggggagg, ctttccttgcactgct, tctctattacggataa, cgttaaaaatctttta,
gttcaaaaatttcgag, gatagaactgatcaag, cccaatgtaaatgggt, caacaaagcatactgc,
tcgtggcccgaggcag, cggtagctgaagcctg, gtgctcaaccaacaaa, aataacggttgcagtt,
atagacagctttgcac, cttccaggagcgcggc, tgtgatgactgtcaga, cttctgcctaatatgg,
ttggaactaacgcatt, ttgtagaggtttgaag, acttttactgtgcttg, ttatcctcccgcttta,
aagacgtttgagagga, ttatgggtccctgaat, gcatagtaattgatca, ggtgtagatagctgga,
gtccacctcttccccg, gggcctgcccgaggcg, cgtccagccttaaatt, atgtaagtaatggtct,
gtcgtcctcgagattc, cgtccctcccaccagt, cccattagaaccttcc, gaagaacttagcactc,
gccacaccgccacccg, taatgcagcaagtccc, ttttgtcctcctatag, gggcgctattttttag,
aaaaaactcgtgttat, tggcaattgcttacct, aaatcattatctgtct, tggagttgcatctgac,
ggcaaccttctcgttt, cgtagaacattacttg, gagactcttaggcaga, ccatgattgaactacc,
gagacataagtgtccc, tgtacacgccccttga, agttctaatcctgcgg, gtgcgactgtgtcata,
tatcatcatttcgagt, attctgtcccttaaag, agtggtgccttactta, ctagtgaaacctcgtg,
ttgactcgaaaggagt, ataggacgaaaattgg, aatttggtcgttaaga, ttaccaactgcaacta,
gatggtgtaaatcgtg, agaaaccgtgcagtgt, cttgggtaacctgttc, tatgcaaggcggaact,
gggcaacccagttaga, ttgaacccccccacca, ttccgcactgccccaa, ccttagccacccgtgt,
gcttgccctctgaggc, tttctaatttacctag, gatcaaaaaaaaccgt, agagattgtgacgtta,
actgtggctggataat, cttcccaatgtttgac, ctgtatgatattattg, ggcacaaacatgcacc,
gtccattcttcaaact, gcgtgtaaaaaaaatc, attgaatagataggtt, attggccccccccccc,
cgggaagagtcgctcc, gatcatttggttttgg, gcctaatccttaaaag, tgttttaacagctcgg,
atggtaaaaaaagtgg, acccaagtactcctca, gcagcttagtgggcac, gaaacgcgggcgggac,
tatgatctattgacca, ctcggtgacaatccag, ccgaaaaaatggtgaa, atagagagagagttcc,
ccttaagaatattctc, tgcattttgggagccg, cacttcgtttttttgt, aaaaaactagtggttg,
tctgacagcgtgtaac, gtgtcaaaaaacgaag, agagctaaggggggac, ccgtctttttctggta,
ccttggggctgcgggg, ggtcaagctgggcgag, cttttctcaagcgtct, aggggttgaacaccag,
gaggggcaagtcctgt, aaaactagatggcacg, gcgttttttttctata, gtgatgtaagaatatt,
tgcttgccggtcgtgc, cattcctcgccgcccc, gcgtgggcgtgtgcgt, aaatccatcatattcc,
tccttgtgagcctaga, cccatgtagccctcat, ccctaaaccccatata, gagtaaactacagcaa,
agtttaacgaatgcta, tctgcgtgaaaaagcg, cgtaaaaaaaagggtt, aaattttttcacgagc,
cgttggcccgactggt, ggaagggagtaagcct, aacggacccaggctct, ataactgtcgtttata,
caggtgcagatatgag, agaagcagtggatagt, aattacttgcagacct, gggctgtttgtagtgg,
ggttcttgatctccat, gttatgtagtcccttt, cctcactcggcggcag, taggacaagcacctga,
ctcctatgtactgtca, gcacttgactgatttc, tacgcttatttccctc, ctgtttcgagggctca,
ctttctgtactactca, actatgagcattcatt, cccatgtagtaccact, ggacgtttggacttta,
agcagacggcactgtg, agaactgcaaaccttt, atacttagtgtcaatt, ggtgaagacaacttag,
aaatttcctgcgtaga, cgtgaccgtttggaca, gtcccttttttagtac, aaatttcgagtctcct,
catttttcccccgtat, cacctgcacaaattga, tgtcgctggatctcct, tatcagtatgcaatat,
gacagattctcgccta, ttgggttatcaattca, ggtgtctggggtcctt, gattgaactacctccc,
ctagcaattaatcaat, tccatctcagcggctc, ggcaacctattaaaga, gtaggagaacttcgtg,
aagatggcgccgggat, tccgctcgcggccttg, gcctccgcaggagagc, gttagggagttttaag,
gctctaaagttacctg, cgactccggggcaaaa, gcggactgtgctttac, cgtccccgcacgccct,
tgtgcccggtctacta, cctaggcagtggcagc, gtaagcctgtcttctg, gctgcgtttttttctc,
cgggatccccgtgtat, ggttcttctttagtga, ttaatatggaataacc, tagagtgggtgccttt,
catatatgatgtctcc, tgtgccagcaacccta, gtggattagtctaaac, actacggtatcctaag,
gctagcgctataactc, cgcctacttacacaga, gtaaaccttatacatg, gacctgaagcttaaag,
gaacgtgaggagcgtc, tgtacagggaatatca, gatctcacccactggt, attcgattttgacata,
ctcgcgatacatctca, gatcctaaatgatgcc, ggtggcggtcacctct, ggactacatacacctg,
aagcaggactatatca, tgtcctacaggttgtg, cgcttgtctaagcctt, tatcatttacgccttc,
gttaaaaacgttacta, aggggctgtgccagcg, cataggcttttggggg, cgggggtcccctaaca,
acgtcattttttggtt, acaaatgaggtgtccc, acggcaaggggggata, atagcaggttatgtca,
aagggcggcaattaga, tttaggggggggtata, tcttactaggtcgata, agtccggcaggcgccg,
acagtgcatcatcaat, gttcgttctctgtaga, cataagctcggtcaca, tgtaaggcacccccca,
tgttagacaagatgta, tccacgtttacaatgc, actgcagctttggctc, ggcaggtaccactgac,
attctgtcttcctaag, ttcatcccgcaccagt, actcaggggccgcgcg, agatagtgtagctccc,
tatcgtagagaaaaaa, cgttcattaggcaggc, agcttggtctggccgg, cataaaggggtggatc,
gcactctccaccatac, atcagcttgtactgac, atgttaccactttttt, tatccggtagaggtga,
aatttcgtggcctccc, gctagatatcctgtag, tcctatttcaggcagg, cctcaagcttttctca,
cgttttttttattaac, tgcaattgcaaagagt, ctgggcaaaaaagtta, tacagtttaaaggctg,
ccacgtatcagtgata, ccgggagagtccgggg, ggcacttgccgcagtc, acgaaaaaaaaactta,
agctaagagaccttcc, gtggtaaacacacatt, gttgggatactcaggc, agctgggctgtttcga,
aggcctaggcttgggt, ggtggagaatatgcat, gtgccagtgttcttag, gactatgtccagctaa,
actcaggcccccgggc, actcgacccaaatctt, cctattcctatatgta, cgggatgctgcggcag,
ggttcttatagatgag, tatctgttgaaaaagt, ggcagggctaaatgac, ccagaattcttacggg,
tgggtgtgtcttagag, gccacttgaggacact, ctactaacccagttga, gtacaggcctttgaaa,
cgccagattttttttg, gactacatacacctgc, cttgcggacaacccct, actagcagttcacaac,
agtcgttgcttatgat, tttcgagggctcagag, ctcggcaaacttaggc, tcttctactacaaagt,
cgctcgcgccagcagc, ggtggtctgagcctaa, agcaaacctatttcga, ggtgcccggcgctgag,
ttagggtcttcttagg, tttacccatagagagc, aacacacacaagcgtt, tgccttaagggacctt,
gcaatactccactcat, taggagagattgcaac, ggcagattgccttagg, aaacgcgggggggact,
gatgggcctgtcagta, ggctgctggtaatggc, agcttaatccaaccct, agtaactaacaggcat,
ttcaaaaaaaagtggg, tgtgatcccccccctc, tcgtccccagggttcc, actatatagtatgaca,
tactgctgaatagacc, atatgagctacatctg, agtttacccccccccg, cttccccccccattcc,
tcaccttgagtggtga, tttttaactaaggatc, ccggaacagttatctg, tcccctacgtcatcct,
cacgtgagcatacagg, actgaatgccattacc, gagtccataacactgt, cctgagtacgaatgct,
gatcttggggttggac, gatctgagcatgcttt, atgtggaatgttagtc, accacttacaagggca,
tgtcacggaaaaaatt, cctatatgcattctgt, ttggacagtacggtga, ccctcgtctgagatgt,
ggttgatacaacagcc, atttcaccttagatgc, ttaactggacccaggt, gttccacttaactgga,
gtatcatgttattgtg, ggcgcggccttgcgac, gcctgttcccggccgc, tcctaaagagcttgat,
gtccgggggaaatgcc, ccttctaactggcact, agacagcacatatgag, gagtcccctttttttg,
accaaaccaccttgtt, tagttttgaggacaat, agcatgagtaacttag, acccggataatagttt,
tgctgagacgatgtag, accagacgaaaaaaaa, tccagtgtatgattct, aaaacctattgtggcg,
aagctgaagcactagc, cgggttgtatagtact, gccttcaatacacact, caggaacttgtggtca,
accgtgctcgagctga, tgtgaagtcttcctgc, ctcagcttaggcagga, tgcaagatccttaggc,
tgtaacagcatatctt, aatactccttgttgta, gaagtgtttgagtatt, agaccttagtagtgca,
ctaccctccagagaac, aatgtcgtatttccat, agggtttaggggccta, tctgtcttgtcggcag,
gtggccccccttacta, ttaaagcaccagctgc, tgaagggcccaatact, gtaaaagcctgacatc,
tgtctgaaacagttac, gtgatcatctattaga, tggatcccttatttat, aacttcccaggatgta,
gctctaactcgtcaag, cttcggtgtggtttgt, aacggcgcaccgtgag, caggcttgtggtgcga,
cagagtccagggaccg, caaacctgaaagttcg, ggcctttttttgcccc, ttactccccttatcgc,
tgtctccataaattga, ctcgtctggagtctgt, gcttcacttaggaggg, ccatttagtaccatga,
caacctccttcgctgg, ggacactaaagtagct, accactgcattgccgg, agaggacttctctact,
tccatgcagtgatggg, tatctgtataactcca, ctttacacagacttcc, agctcgcttctgtcct,
ggcttacttttcctgg, cccatactttagctgg, gtatggggtttaggga, tatacagagtattcac,
ctccccggggcacctt, atggcgatatttataa, aatcttagacagtttc, gcgacagttagactgt,
gagatgtgtgcatagt, aagtacagttaacaaa, ggtccaacaggagatg, ggacggcttcagtggt,
tcttgtcttacttgta, aaccccttaggtcaag, tgcccggcccgtcgcc, gegcccgccttagctg,
ccacctcagcgacacg, caacttagcttttagc, gtcaaatacacacgta, agcctgcatatggcca,
gattggtacactataa, attatggatcagtgcc, caagtattcacttagt, ttgcacttcctctcga,
tgacgatacgcgagcc, atggggggtaataata, atgcttcggtgtggtt, tctttactcgcctcaa,
agagacgagcctcatt, taattgaactagtagg, ctgaagtcttgacatt, cagtgtctagtctaag,
gtctcttgaccactag, tgtgtccagcagctat, ttgcgtttgccctggg, gcttaggtgaaggccc,
gtatcgacctcccaaa, gaataacataactgca, tgaaaaaaaacatcta, aggggggcatagtagc,
tttgggtgtgtcctag, aggcagaaatcgggag, ttacccaactagcgtg, tagcatcctctcacac,
caaagggcatcacaaa, atgccccagcttggca, tcactggttatgtctc, gactctcttacatatt,
ttgatatgaagtgttg, cgggtgtgcctaagga, ggcttaagcagagcaa, gagagcaaacggccaa,
acacttgaaggctgct, taagtctataaggact, gatactccccccactt, cgaaaaaaagcataaa,
catagctgccgacccc, ccatgtttagtgaggc, gatacatccggagtga, gcttgcggacaacccc,
gggaatcattggaagt, cccatcatccgatatg, cgatgagaggcctggt, cctctgacccccccca,
tttccggtaaactagt, gatgttaaatccattc, gaaggggcatgatctt, atattctggagcgaac,
aacaaaacgcagacca, aatacaagcggattct, actgtcctactaggtg, tttctagccatgaacc,
ttcaaaaaaaatccga, agaagccggggggtag, aaatataaatgcgaat, ctaaggggggacaagc,
gcaacagggagctccg, gtccccgtgggtatgg, aaatacgaggtaaatg, aggacagctgttatgc,
gcccaatgaaagctta, tttgggctaaacagtt, tgccatttgtgtaccc, cagtgggcagaacgtc,
tttttgttgatccaac, agatgactgaagccgt, ggggttagcctttgca, aggagctaagacatca,
aggtcttggccaatga, taccggccttgcctgc, gcatagggcaaagtca, ttcccaacctgattga,
tctgctaaagagctat, cctcactaaacatatg, cacacaaccatacttc, tttgacataaagggtc,
tctattgtaaagacta, tatgttagtttaacag, ccctagatggtgggtc, agactagtgagcacta,
ccctcagaattagtaa, atggtcccccccgaaa, aatatactcgaatcaa, gtaccctaaaaaagag,
tccacgttaaatcaac, aaaaaacggggggttc, gagtgttatattcccc, tccctctcgtgcggag,
tggggttagattcctg, ctctttacagattcag, cacaaagaaaattccg, cagccagtcagttgtc,
cactttgctgggccga, gggcggcttcctcgct, taccataaataggtta, actaggccgccgacaa,
gcgggcttagtccaaa, ttgtgcctcgtcctcc, gatgagataatcacta, aagcctaccccccatt,
gaaccccccacgcacc, ctagtaccttgtaaga, aacgatcattacatac, actcatagttgacctt,
ttaggcaggcatgcat, gtatgggccacacatc, cttctatcactgatta, caggactaagtaaggt,
gctcccccccggccaa, ctttgtatgcatagta, ctgcaaggagagttag, tgctgtatagtgtgtc,
ccggggttcgtcttct, ttcctatcagggccaa, ttctaaaaaaaccaag, tatctctacaccctct,
agctgggaatttcgtg, catactctagtcagat, aggatgggccaaggta, cgactcaacacatata,
ggtgtaggggaattga, ccctgtgacaactaca, attaggtgggggaacc, tggtaaaaaaagtgga,
acttacctgattaaac, aacaggtatgggggga, gtgggggggacggtag, gccccaaccgaacttt,
ctcctgaggcactacc, gtggccagtcaagacg, gatggttcaaacataa, taaaaagtcgcaaaat,
taaatgccccccctca, ctcgtaaaagtcatgc, gcaatctaactattag, gaactgtccacgttaa,
atgatgacgggcattt, tttaccttagctgcag, caagaagttcatcccg, taaccccaaaagattc,
tcatttttgatacacg, agggcaatggaagtcc, tgtcagtaagtaatat, gctccattacatgtgt,
aaaacatatctaaccg, aaggatacgttattgg, aacacaaagcgacatt, aacctacgtgaaagta,
ggggaggtaggttttt, gatgcacgggctcttt, gagttaccctgatctt, cacttaataggctgat,
tgtccatgtgatccag, gcctaagcatcccgag, gtgtccttatttaaca, tgtttgcgtttccctg,
ctaggggcagggctcg, tgtgctggttgaaatc, caaggaatatttgccc, cgagggctgcctctaa,
gctactaaggcagctt, atagtttactctactg, aagttgaacgttgcag, ggattcatgctgatga,
ctccctttatataacc, tcctaacaagttcgta, agttttccctcactgc, taccttccctacattg,
ggtgggctgataatga, tgtagattggggtgag, cgttcccggctaactt, cagttgtggtgtcggg,
gctttcccctacgtca, cttggttcccccagga, cgctttctttgagtca, cccccgaaaaaattgc,
ggtgccgggcggggtt, gcgtagcgaggcgcgc, tagaggaaccgcccac, gatggggcaccacgag,
atctgaacactgagtt, atatagattaccccag, tcaatgggggcaggcg, gaaaaaaatttccgtg,
ccacacttcgaccacc, cctggacctgaagtcg, aggggggggtcacaca, cagcgttatgcctgag,
tcatgcacgccttttt, ctcccagcggcaggtc, aagccctgctgtagtt, ccttactccctggcta,
cataagatccctttca, gcttagtcttattagg, tggcttatgaaaacag, caggcaacagatacat,
atatgcctttgactga, gcctggacccagtagc, tttctttatctcggaa, gtatgtcctttttttg,
gtaggcatatcttttt, acagcatctgctatcc, gtcaccatttggccag, agcaatcctttagtga,
ccacctggcccccgaa, gtaagaggccattcag, acctggttcagcgcat, cagtcgctttttcccc,
gaaacgagaaatttca, gcgtgtggcttaacct, atagccccccctgtga, tcattatgttcaagca,
aaaacgaagcccattt, ttattgaaactaccta, tccggcctggcgaggt, aacggggggttctctt,
ttcatagggtgggtca, caatcgtggtgttttt, ttcgacacggcttcta, ggccttaatcttttat,
tactccatggaaatct, gttgtgagttacaccc, ccagggctcagtaaaa, acgcatgcaaagcaag,
tgttcaactcctggta, atgccggagcagaact, gtcgtccccagggttc, caactatgagcattca,
cttccattggaacaat, ggtgaggagcattgga, agaagcatgtaattac, tgcaaggggggtggta,
gaacgtaggagagtgg, acaatacacttagata, ctgaatagttacagtg, tcttcgttactgcttt,
tgaacctggttcagcg, gctgttcctaaaatgg, tccttcgactcttacc, gttggcaagactctgg,
cccacggccggcccag, actccagggtccgagg, ttcagttgtttgggta, cgcatgcaaagcaagg,
tagctactaccaaccc, taacgagggagttagt, ccaccaacgattcaca, gggtcttgatgaagat,
caaagctatgtttatc, atgcatttctcggatg, gagtgagccaccggac, ctcagaaccagcctta,
tgacacttctacctgc, tctaccttcaattccc, ttgggatcactaggtc, tgggtgccattgcaca,
aggtattcttatcata, atcagcggttcaagac, tggactctaacattag, tccctattagtattga,
agcagcctctcctata, tgagacgtgtggggaa, gcttcaaatacgtgtt, cccaggtttatctcag,
agagcagcattgatat, acatatctaccttgca, cactgagttagttgtg, ttcaccggaaactgta,
ccaatagcatacaatc, ctccgacagattatgg, gagacaaaaaaaaacg, aggtatgtctaggcta,
tatctgtattgaaggt, aagtgattctccacgc, agataaacctagtacc, tctttacttagtggca,
cccccgatacgagtct, ccatccgtgcccataa, gaagcccacaccatcc, ttcctgccccgggctc,
cacttttttttcgtca, tataccaggcttttac, ttgaaccccccccaaa, gtgccacctgttcatg,
ttatataggtgaaagg, catggaaagagtacca, gtacactacaggagac, catataaagttatgca,
ttattaatactggtgg, cccttaggggctctga, aatgcacctgctcgta, gtccaccaaacgacaa,
ctggaaatgagcgtag, gttgcttcttccgcca, ttagtacgtttttgtg, gtctagttcatcctcc,
gtgactgctcccatat, tctctgagaccaatag, aggtacgttaggtaaa, tgctccactgtgcctc,
gaggcctgttggtctg, attaattgtccttagg, acccatacagcttacc, tgtatattgcagctct,
ttcagctgaagagcct, agtcagcttagatcac, atcgaccataaatgtt, gaactctcctgagtga,
tgatttctcaaaggta, gcctttcatgaagttc, aaagtctcaagccttc, tgaatacttccgacaa,
ggcttgtgctggctcg, tacccttagctcattc, aagacaccgatgatgc, gcgcagggagggagaa,
gggtcctcgcacccct, acgttaaatcaacaga, gaagccggggggtagc, ctaacaacgagatgat,
gcccacaaataacgcc, taacttttgggattcc, tacccccccttttgtt, tgtatcttataacaca,
atctcggtttcggctg, ccttagggcattgcct, tttgtaggtccagtac, gccacttagccccagc,
agatccaaatttacga, tgctgcaacatgagga, tccctcaacattgtta, taaacggaaagcagag,
ggactgctagacatca, gcgcaccgtgagatta, gtgtcccccccccttt, aaagaagaagcagccc,
ctcttcccccgggggg, aagagactaagccaac, caaaaaaacgagagta, tctcttagtgaaattc,
atttttggcccggtgg, ggttactttttttgca, catcgaggtctagttc, gtcatacatggttaaa,
ccacgtgctctcccat, gtaccctttttcctgg, acaacttcagcaatac, ccgaacaatgaaaacg,
actccagacgctccgc, tatgatccttaggaag, catgtacattatgtca, ggggtgtttgcagagt,
gtttttttttccgaat, cagttacgaaaaaaac, aaggggtaaggacctg, actgtaaggtccagga,
attagcccttagctct, ggtgcgtgcatcttgc, cttaaggggctccctg, tcctacaatgaggtaa,
aattagtgcctttggg, cccactcgtcacttca, gccttacattcctgtc, gaaattaggccttaag,
ccgtcccccccccgaa, ttcccctccgtgttgt, ggacttgaaatcaggc, atctcaaatggtgtcc,
aggcccggtgggcgaa, gatacagtgtctgtat, gtggcaggtatttacc, aaattagctaggactg,
ataacttcatcagtga, ctcctctaggtggcta, tgcccctgagtacgaa, atacctcattgccaaa,
ggaaaattaatcgacc, gatccatgctgaaaac, cacttctctcagtggg, gctattttttaagggt,
ccggtgtatttgtgct, tattgaggacttggtt, agccctttttttaacc, cctaataatgatgggc,
gcaccagggtgaaacc, gtgcaccggttttcag, ttttgatgaacttgta, cttcaatgctaagtaa,
aggattatcccttttg, ccaatgcacctgctcg, gagttaacgaaagaaa, acccttttagatggcc,
accatacaggctgaag, gctcccaaaccaatgg, gatatagaattttcgg, ctccaagaccgactca,
catcgtgctgattcca, ttttcccttagttaga, ggtgctaccactcacc, tcagcaagtccctcgg,
ccacgacaggaggatt, aaccatggaggtctta, tgcgtctgcagttagc, gtaggcaggtcaaggc,
cgggaactggagaagc, atcctacatatgttta, cctcctggttggcgct, acttagacaggaggat,
gggtgacacgagttag, ctagaagacagaggta, aaacgatgtaaaaagg, cgaggctggggcttcc,
taatatgcatttctcg, aaatggcccattaggg, gccagcttcaactcca, gcgggggggattctct,
gtcccttgtatttaaa, tcatagtaagggcata, agatgtgagaggacaa, cattggccttaagttt,
ctcacatgggagggta, cctcgtttcccctggg, gggccatttatgcttc, ttaactaatggcacta,
ctttcatcggtctaaa, actcacctccttaagg, cagaaacaagttatgc, cctaaattaccctagg,
tcacagatttaattcg, caatttaaaaccgaat, tttagagatcccagtg, tggttaggatgctgtt,
aggagctcaaggtgaa, aaaacgagagtataca, cctcacatctaacata, aggcagtgggctagta,
ttgatcctccctcttt, agcattagagtcactt, ctagctcatggatgtc, gaattatgcccctgtt,
cggctctcttccctga, cggttttttttatccc, cttccattgcccggat, ctcgatgagaggcctg,
cgtgatagtgcctcta, atctgtgtgaattgag, gagaattttcttgcta, acaacttgtcctagga,
gctttttttcggccac, ctcatttgaggtcact, ccatgtgcttatatac, cgtgccagagggcgga,
ccttttaaagattcga, cacgcatctgatgaac, acatacccagggtcag, gtgtgactcttaggtg,
ctctatccttgaggcc, gtgtgagctgagttaa, cacgttggcccgactg, cgatctgaggatgctg,
ttaaaaacgttactaa, acatctggactctttg, ctacagtaaggctggg, caacttgtatgtctca,
gaacaggatagttgga, atcatcaccgtatatt, ttaccacggtcacctc, ttccctctggacctaa,
caaattagatgacagt, aggcgccggtgtccgc, gttacaccctcttaag, tatctaaccctgttta,
taatagggtcttctgt, gaggcagagaccctta, gtgtctgccatcttgc, cagacccactgcaaga,
gcactaggctgccgct, ctccagttagaactta, tttaatcggttgtctt, ctctagtaaccaagtt,
agcgggaggcctggat, acacggaatccctact, agtctccgcctccgca, ccctggcgctgtgtgt,
ttcaaggactagttga, gaatgtattgtggtgg, ctggatggtgctctta, gtcccccgcagcgtga,
aagatatgttacctat, ttataacgatcattac, ccaggatgtgtattgc, gtacagatcatctaga,
acggtgtctaacacct, ttaggttatggcttcc, aaattccgtactgatg, gacggttcctggctgt,
aggcttttcaggtcaa, ggtgaatatggaaaca, tacactatgagaacta, atcaacaactaaagca,
agggaatagtagactt, aggtctagttcatcct, ttgcagaaaaaacgca, gtgtcctatcccttca,
actgataccaagtgta, ttgtttataatgcact, taacaaaccctataat, tggaggggatgaccat,
cttgtaatgaatcacc, ttctctcccccccctc, tcttttacgaaagcga, ttttctcacccccgat,
ctcgaaaaaaaggaga, agtgagcatagggatg, gagacttttatctctc, gtactttgtcggcaat,
cctgagggcctatggt, aacgggggggttgttt, ggggaaccaactgagg, gttgctcaagtgctac,
ctgtacattggtcctt, ttcaagcaattgcacc, ttggcaatcagtagtc, cttcagcctgttttcg,
tctagaccagctcggc, agccagcagtaatcta, agcttttaccctcgct, aagtgcttgataagac,
tggcctaaatggctaa, cattccacttgtacga, aacgtagttagacctc, tagtagttgttaggga,
tccaacgttttctatt, gttgagcagctgaggc, ctagccaatatgtaga, ttgacattaacccccc,
aatcgtgtataaattt, attagtgtctgtagga, ctccttagatatgtct, cttcccctcttaggag,
gatgtgccaactttgg, gtccccctatctggga, tgtttcatagatgtgg, cttgttgttgccactt,
caccacccaataaccc, ttaatgtcatgattcc, cgtgcccctctcgctc, gagacattttcgctct,
gatatcctaccttagc, ctacaactgtattgta, catgtcagcgattggc, aaggtgcttaattcta,
gtgcacgttactagtc, acgccactccaatccc, ccaccccccccacttt, aactagctaagagcca,
gccctgctgtagttga, cgatgctgggctcggc, ttttgctcgcccgccc, ccatggaagatattta,
ggcacagtagtcctgg, ttgagaaaaaaaagcg, tactctatgaacttgg, cacagtcggccttttt,
actaatggcactaatt, tttttacctcgaatga, acaggaactccgtatt, acatccggagtgagac,
gaataggtcttcactt, gcacccccttattagg, ggcccctcgacgcctt, ggaggtagaatataga,
cacaaaaaaaaggagc, cgaaaaaaaagaggtg, cctcagtgttatctac, gatagaatgtcttaag,
tatgctgccccacaat, atctggatgcactcca, taggactctgcgatgc, aaccgtatattataat,
tcctatagttccactc, gcacttgaaggtgaca, gtcatacaaagtgcca, gcaaatgttgggtctc,
cagcaatacggtagtt, taaaagaaatttgcgc, cagatatagccccccc, tccctaagaggggcgg,
gccacgatggtcggct, gttccccagacggact, atgccgagcctaagct, tcttactgattgctag,
tccccccccacactct, acatagctcccagaga, tcatgctaaaaaaagc, cagcaggtgtcgctta,
cttccccccccatatg, ttgttagagtcgggtt, attgaaattattttcg, ggggtcttaggcaaca,
tctgaccagtcaatga, attctccagcgcaact, cgccaactgcatgtgg, gtgtgccgcaccaatt,
atcggggatcctcaga, gatccactttccggtt, acaaaaaaaggcgtat, agttcgatgtttggct,
gcgcccccccccagcc, gctctaccctgcttgt, ggtgtatgatgacact, ggcttaagtctgaaag,
ttgggtggatcacctt, cttcctacaagcaagc, tagacctgagaacccc, ctcgtctgaggcttca,
tagcgttttttttggt, tggcccccgaactatt, gagggattagtatatg, ctttagacacattgct,
tatgatgctagctgcg, attcatacacgtatta, attaggtcagcttccc, ctagggatttgagaat,
acaagaaatagtgggt, tgtgtcccggtgattc, gacctaccacatacgt, ttataggcatagggaa,
gctagggtatgatgag, taacgtcacgcttttt, acagaaaaaaaagacg, ttcatgactatagcat,
tgtcccccgcagcgtg, aggttttttttaacgg, ctgctcgtaaaagtca, gggttggctcctttta,
catgcagactagagct, ccggacgtagtggctg, acaccctctgtaggct, tgcccggcggcatttt,
gtacctgagctgttcc, gcgcttttttttggtt, cactattatatattgg, gagctcgcgattagcc,
ccccctcttttttagt, ttccagagtcttgttg, agtcccagcccccccc, attagggataccagat,
tccccaggcaacttta, ttgaactggcaacagt, ctaattctggcacgtt, cttcgcaaaaaaattg,
accggaacattctgca, cactttcccccacagg, aaactagatggcacga, acacattaggctgtgg,
ctttagtgtagacttc, cgggacgggtgcgagt, cgcggcagcgggaggc, accttcaagggagacc,
cgaacttcctgctgct, ccccgcacacacctga, tacactccacacccct, cccgcccctaaacccc,
accttagtctctgaag, gaattgatcatgtgct, atgattgaactacctc, atatctaagggaatag,
tgcgtgcgccgcggga, tcgttatctgtggtta, aagcactctaggatga, agcactctatgctccc,
gctccccagtggcgcg, tttaggaggccttcct, ctagctaaccttagct, aactcactgagatgag,
ggtaattgtacaacct, gtggcctgcttggtgc, gagcccttatgttcag, tgaagtgcttgataag,
cactttaatgcccccc, gtagcctattgcctta, gtgtaaacaggcaaac, gctctgcacttttgaa,
agcgcctacttacaca, ggtatgacacactcat, gagcctaagctggact, taagcctcccgagttg,
cgggcgggttgagagc, cggggacctccatatg, cccaacaaaccctggt, gctttttttagcattg,
tgcccctaggggcacc, acagtccaggctgttc, ggcggtggcggggatc, gtgtacatgccagtcc,
gactccctggttttag, tgatatctatcacttc, cccggtagcgtgaagc, aattaggctcccaagt,
cgctgagaggtaatgc, tccctgtattctacac, gtggcacattttggca, ttaaaaggcctagtgt,
atggcttaaatagata, gtgaggggggtactat, gaacgtgggggtctcc, agagcacgttaggctg,
ttctaaaaaaactgcc, ggttcagtctcccttc, gtgattgtgcaactct, ccactcctgtatgtac,
ggagtccttatccatt, tagtttgggaatcacc, ccttcacggtaatgac, ttgaacatactaacta,
ctttgttgtacaagcc, aaggcaggccacttaa, attaatgtcgcatcta, gcgtctacacagcgca,
gatgtgaaaaagtcac, ttgggacagctaaaat, gcatgtgccttaggtg, tggtgtcccgggacgg,
cttttccattgatcgt, cttaattcaggagttt, ccagtgtgagcgactc, gatgtacttgtcttct,
atgtgaagcctattgg, tttacacttattgctt, ggcttagaggaagcga, cgcacagctccaatat,
tataaaaggcccatca, gggctacccccccata, aaccctttatgctaat, tttttttcccgaggga,
gtattacagagaacct, cacctggcaagaagaa, aaacaggtatacattc, tgatcctcctaaagga,
gtacttcttgttggct, cccattatctccaacg, tgataagtctatggaa, aacggcggagaacctt,
tgagtctgaaacgagc, tatgtatggtccattt, aattattctcagatgg, tccgtcccccccagaa,
ctatatccctatcagc, ctctggtggctttagt, ttacccagtatcaaat, tttccaacgcccaggc,
gagacagatttccggt, gagttagactctatca, tatgtgaggggggggg, ttgtccggagaatacc,
tgaattaggaggatag, atgaggcctaaaaagg, gatgaggtacgggcgg, atgccgttttttttac,
agccctcttccgccca, tttctcgaatggaaat, cggggagggacagtat, cctgagggtcccttgg,
acccttcatctctccg, taattcactgtcagcg, gatacagtcttaccct, gggacccccccagcag,
tgatatcgtttattta, tcagtttttaccggta, cttaggtatgctcatg, tatggtagcctaaaca,
cttcatcaaccttttt, ggtccatagctgccga, gggcttaagctatgct, agttaccaagatttcg,
gtttgaaaacatgggt, aatggtctaaaaatcc, cacaaagcgagcaaga, gagccccgctgtcttc,
ctaagatttgagacta, gttaccaccatccgaa, aaagcccgggggtccc, cgccctgcacccgcgt,
gctctggccttggcat, cccaggtgtttcagta, aaaccgaatttttttg, ctattagttaactttg,
aacccccaaacaagag, cccctgctgggaacgg, ggggtgtggaagtctt, gttggggggggttcac,
gagttaagtacttcag, agcgtagtgactaagc, tttcggctgaagtcag, acgcggggtggttgag,
tagatcacgacccccc, acgaaaaaaaaaacga, gcttgcagtgatgcga, tggtccgaacttcctg,
agacaacatctacgtt, ccgagtgaccgaggat, acttgaaacaaaacgc, taaggtacaggttatg,
atttcctagcttcaag, tctcaccctccgcacc, ctaatagtacaggtat, tgtacactctgtgtgg,
tacacacgtaaagggg, gcaaactgtgcccccc, ggatttgttcctttac, cccaattgtaaatgca,
actactccggaggctt, agataaattcgattaa, attgcgccctgcactg, ctcaagcatgaacact,
gtttgttggctcactg, tgtgtgttcttaacga, gaggagctttattatg, gcaggccaaccaccca,
gggggttccgcccccc, ttacataggcctaccc, tttaacgaaaaaaaat, gcccccccccagtcta,
ttgtcatcaggcgaag, accgaactttttttag, gggatgaagcggagtt, gcatagagagtgaact,
cttaagaatggggctc, tagcagcactaggtta, acagtgtaacaaatcc, aatgtgaccccctctt,
acaccacgggatatgc, cttgcacgtcttatta, ggtatgtactcttaaa, tatctttacagaccgt,
cccccgcacacacaga, caagcatcaacagcta, atgccgcccatgcagt, cagcggtaaaccactt,
atagcagactttgaat, caattttcgtgacatt, taaggcagcttaggca, tcagtaaagttctgaa,
cggtgctgacgaacgc, agctggaccagatacc, gcgctaggcaggatcc, taggggggggtataat,
tgccatcccgagcatg, atggggcaccacgagt, gtgtggcttagagcca, ctctgtcctgcttgcg,
acgagagagagaaaac, tgaggtactaagccaa, aattttgtacatgcga, gattttcattgatctg,
cagtatggggcctggg, gcttcttctttattag, gggggtatattctgag, gcccactaacaagtga,
cttgtgtggggggtag, tatgccggcgcttttc, cttagaaaaaccaact, ggtgtaacagtagaac,
cagatgtgtccttgtc, agctgacactatttat, actcttttccatagac, ggtgatccttttttcc,
cttttctaggccgagg, gctacccccccatacc, gccatttagcccggtt, cagtttgaaggcacat,
ctcggcggcagctgtg, tttgtggagtattctt, aacccccatgggtacg, ggcagatagggggagc,
ctcattagtagcgtag, ctgttcccaaacacgt, aaggctactggtgcac, cttaataggctgattc,
ggtgatcagagaccag, gtgaggttgcgtcttg, gggggcattcatcata, gaggcaggtcaaccta,
cagaggcccagtggat, gtttttcactgggtgc, ggagatgcaaacatca, cttggtggcacgtgac,
gagagcatgccggaca, tgtaggtaatcataag, ttccccccccccggtt, cattccgcagacacgc,
cgaaaaaaaaccatgg, cacctcatacataaga, cttgcagctgcggtca, atactcatgggcaagg,
atgacagtaaggctta, ccggttcccttcaggt, aactcgtaagcactca, gcttactaggtgtgac,
gaaatctgaccctgat, tcaagatggttaaacc, gcaccgaggggggggg, ccgcactcttcttaac,
catgaggtatcctctc, tatctccaagccacat, tttcccctacgtcatc, attggttacacccgct,
ggaaattattgtggag, cttcaggatgattcta, acccctccagccacgc, tttggcgtgcactatt,
cgtggctaaacatctt, aggaccgcaggtctca, atagtttaaagcgtta, tcttagtgtgatgctt,
tcttacaggcccagca, gtgttcaagttgtatt, tctaattatcttgttg, cgtggcatatacaaaa,
tgtagtaaatccttag, gacttgacggttcctg, ttttttaactccgtct, tcccgcgaggcctagg,
tccccttagctcattc, ctccagcgttcggcct, cccagaggttctagcg, catcctgagcttacga,
ttctcccagcggcagg, aacttcggattcatga, gcccttgtgttgtagg, ggtgtgcattgatttt,
tcagctttaatagtcc, taccaactaaactccc, tgctagagagctaaac, tgccagtaactgggga,
tgttcttatagtagga, gtctttatgaggagcg, ctccctggtgcttaat, caatgccgcctgccag,
tctggacgactggaga, atccgttctgagctcc, acggggccacaggtgt, gagggggggggaagca,
tacgttctatatatat, actgtcagacgaagga, gtcttgtatgagactt, gtgtagatagctggag,
ggtggcttaggcgggt, aatccgatgttctagg, cctagaagctgataat, ggggaagatacaacac,
atccttgtggagagtc, aatgctttctccgtgt, ggttatttgtgggttg, tagtgaggcaaaccta,
catatggggacgtcga, gcccgccacaagtatg, aatggctctcctaaga, ctggcttacgtgcgcg,
gtgcctcctgactctc, aggcggtggcggggat, ccgtttagggcaaagg, gtcctcctgtagctct,
gtcaaaataatagctc, gggtttaacgtgttag, agcttacacaagcaca, aaattacacagtgaac,
tagccacccagatcag, ggaggcgtaggttcca, cccggggggagacgat, tttatgctggcggaaa,
tgcgataatgttagat, ggcaccaccatactat, ctaagactctctggag, gctccacaaggtttga,
tcgggaatataattca, tcttgtcggtggaaag, cagcttttttttgcta, tcgggggcttaccccc,
gtgctatgacttaaga, aacgcaaagcaagcaa, tccggagcagcgtctt, atttattttatccgct,
tcacccttgaaaggac, tccgacattttttttg, ctatacatatagtaag, tctcttcacacgggac,
ttctaaaagcgttcct, gttgtggtgaactgta, acactataaccagtaa, aaaaacctcgaacagc,
ccggctaggccgccgc, gcaaattgagcagtct, ggctcaataggaccct, ggggggggggattcaa,
ctcgggctgaagggat, tgtatcggttttagaa, attatggcgtctgctt, tttgttactagacagg,
cgtctacacagcgcag, gccagagcattactga, taggacctcccctgtc, taccatagacttaata,
atgggcaaaaaaaagt, gtgccactgtatcggt, cgcggccttgcgaccg, ctgatacaaccatttc,
ctactgtttttttggg, tcagaagctttaccga, taacttattcaccccc, gggcttagtggcgagc,
cctttatatagtttaa, agctagcctaatgtag, ccaccttaggctctcc, atgcttcgcttcggca,
ataataattacatcgt, tgtaggggggggggct, atccagacacgtaaat, tcagtgcatgttgcat,
gagcgtagtgactaag, atgcttaggagccatg, caatcaccgtcttttt, gccaagtgcctacaat,
agaagctatcgattaa, tcgaaaaaaaaaaacg, tctgaccagacacttc, gttttattccctacgg,
cattcatgtctagatc, tagcatgttaagtgga, tcacccccccgacatg, acacaattagataagc,
atagaaaaaaaacggc, cgcggccttggaacga, atgttaagctcatgat, atcaaaagggtgacct,
ttatgagtcaggcgca, gcgcagaagcctggga, gtcctgcttgcgagtt, ttcgctcaggccctcg,
ggcacaaacaactatg, tcacccgctctagaag, tcccctcgcaccctat, gtgagtgtctacaccg,
ctttgtatgttgttag, tggagctagagcgatg, aatctgaggggataag, acgaaatgtactggcc,
taaaggcaaatcttcc, tacgctaaaaaaaatg, gaccccactttgtctc, taggggggggaggaga,
gaaatctgggtggaac, ttttaggggggaacct, ctgcatcattttctga, ccccacatggctcgga,
gatggggcacattaag, tggcttaggcctaacg, aagagggtttgaccct, gcttactaaagcagat,
tgtaccacttagcttt, actgggaggcgtaggc, gctcgtgatagtgcct, cgttgtttttagatga,
tagatgggttcttggg, ttaacacaaggccttg, gcagtcccaacttcaa, gaagctctcacgccca,
ggggcattcatcatag, tctcattagtagcgta, taagagatgcagtgcc, gtcctgggaggagtcc,
tctatgcctgaactga, actgccttctgggtag, ctttgtagccatcctc, ctatcaccagtgcatg,
tgtccatgtattactc, ctaactctatgtataa, acactaaagtagctca, cccaccgtaccctgct,
gggatttacacttgtg, agcagcgctgggttct, ctggtggtcgcatgaa, ggctattagcaattcc,
aagggcagtgtgacac, taggactttcggtatg, cagattagccttcagt, cttaggcttgagaatc,
atagaactagtattta, actttgtctgggatta, ggaaagcttagggagt, tcaaggttggggtaac,
tgtctagacattgagg, ccacgttattctgttt, gagtgcttcactcgcc, cccgcaggatctggcc,
aaagccactgcgccct, ttcaatggcatgcagg, tagagtctgttctgta, gctaacacatgactgt,
tccccgaactgaaact, cctgaacctggttcag, gagaacggggggggaa, gcattcagcaggatca,
caacgacttgtgacaa, ccagatgacttaacgc, gtcgaggtctcgccac, gtttccaattcaagac,
ggatatagtcacaggc, ctcattggtatctcta, ttctgtttccgaaccc, gccggcttagaggaag,
ttaccttgtgaatcta, cgccaagcaaatgttt, ggtcctccttagtcat, aagtaaccgcagtggg,
tagatgggtctttacc, atggtgtggatactgc, gctaaaatcggaacca, tatgttgatggcagta,
agctcgggggcgggcc, actgggtaacccagtc, tccaatcaaattatgc, cttctgacaatagact,
tagaggtgataacaca, cttctgggctacagcc, agatccatctaggccg, agtgggggggggctct,
gctaaagcttttatag, ggtgaaaagagcaacc, gagctctcgctgctat, aaatcttagtacgaat,
taattttttcggtgga, gggggggggtgcggtt, aggactcacacaacat, gcagttagctgagacg,
acctatattgtgggga, ttggcaagtagctaca, cactactcctggttgc, gggcatagagcccagc,
tacggataatttgcca, gaactcagccagactg, gaagagtcgctcctca, tttagatacgccccat,
gcaaacacattaggca, tttccaagcgcagatg, tctcgtaaaaaaaccc, atgcggttgataaaag,
ttatgcaaatattcga, tgaatatgaatagcac, aagctgctactaatgt, cctcttacaagggaga,
ctcctttagaatgcct, gcgtttttttctccaa, ctgccggtgccccttg, tagtaaatttgcaccc,
tagtagccatgccaat, tgaagtgactaaaaag, atttcatactgcgttt, aagacgctgaggaggg,
ccatttagtcagactt, cattcacttaaaccgg, ccggctaaaacggtta, caattacactaccaag,
tgcggacttaacccct, aataccctatgcctta, gagtggccattttcac, taagggggggagtatt,
ttatctccaacgtttc, ctgcaatgaaggggct, gcgtgcacgttactag, ttggacccccaaaccg,
gtgatagatagctcta, tctctttttaggttca, acttcctagtgtggtc, tcactttcttccgtac,
attgagcactatctac, cggataagatgcttag, ataattctagtgccat, ttcacacctagagcag,
cattaatgtcgcatct, gcatcttcttgcgtgg, acctcaatttatagga, acctacgtgaaagtaa,
ggggtggttatagttc, agagaattccatgtta, tgcacttatactggag, gaataaactatctgat,
agcgctcccccccata, tgggcggcttcctcgc, aaaccataacatgtag, gctcctggcctataac,
ccctgaaataacgccg, attgcccggatcagga, cggtgttcgggtcccg, gctgttagaagacagc,
cacctgggggggcgtc, cagcatcatacagata, ttctcaccgatgctta, gcaaaggttgtcatgc,
agactaaaaccctgta, gtgcgttatgaagtag, tccagattcgccacct, cagtttgggccggata,
gaagatcatttgatac, cttcactcgccccact, gatttttggaattgcc, ggtctgccattgctgc,
cgtgccggtttgtact, ttgcgcacttccgggt, ttcggccagtcctctc, cggtacaccacgggat,
gggcataagcagcttt, gtgcgttttttttctg, ggtgttcccgaggaga, atagcgtaaaaaaata,
gctaaactttcttacc, tttagtagcgatgctg, cacagccctcagctta, catattgtctgtctca,
ccaccggaaaaaaaag, aagactgctgttccca, ccggggcaccttgtgc, cctgtcccccgcagcg,
aggggagtgtttttta, catacaacaattatca, ccatctttgatctaga, aacaccttagctgttc,
cttttgagtagtaagc, ccttactggggatgtg, cataagtgggagtcta, acttaacgactatacc,
tctcattagacatatt, ccggccagaggtgcta, cgggaagcttaggcaa, tattgatgtcttgacc,
cgaaagtgagtatatg, ggtacaacatatcttt, ctaccttccctcagtt, aaaagccttagtgggg,
tgtcccccccatcatt, atagaggttagataac, agcgtctgcggaaggc, tgttatgatcttagct,
gcctctaatgaccaag, ccgacttgatcgtatt, gtgagcttccaagtgt, tcgtaaaaaaaaatca,
gtgggtgcttgcacca, cctccaaaaatcaacg, tcgatgtcctgaactc, cttgtttatcatcaag,
atatgcaactgttctc, ggttgagagcaacacc, ggataggtacatgtca, cagagttgcacccgtg,
attatacctcctggca, accatctgtttgaaac, cttctgaagattatct, ggtttccaacgcccag,
aaatttaggactattg, acagcttacccatacc, atattcccacctaaaa, cccgcagcctgtccag,
ttggagagcctcatta, gacccccccatatata, gacttaaaaacaggac, ggacctgagtctgaaa,
gaaatcgaaaccgtct, gactctcggggttgtt, gcctattaagttgcaa, ttaaggcaatgttcct,
aattcactgtcagcgt, ccgtagtcggcgtgcc, ggataagtattcatta, tggggggggcagaatt,
tctaattgctataaag, tagcttccccaaatgt, agtctaagccagctgt, gtaacggtcagaggtt,
gtgcatcttgcgcgag, tccaagttggcaccat, ttgcggcgaggggcag, ccaggtttttttacat,
gtcctcgagattcctg, atgttgtgtaccaggg, gctgagggccgggcat, actaaatttcacacca,
ggggggggacaggacg, tgattaagtgaataga, tcttgtttcgttgctt, cactgtgggggtctcc,
ccgtgcatcgtattac, ggattgagcaaccctc, cacgaaaaaaagagac, gcaaatatgtccagat,
ctgaaacctcaaccca, gccgctaaaaaaatct, attccttcccccacga, tacttagtatggtcct,
tgaaaccacgcacttt, ggatgcgcagtgtaac, actcgggatgctgcgg, gggtactcagattacc,
ggttatggatctgttc, atttactcccaagcta, ctttccggttctgaca, actttaagactgacac,
cagtccacatgaggac, catgcttcgcttcggc, cctgccgggcttaggg, gattagcctagcaaac,
tgaatagactcaggtg, ggtatataagtcttaa, tgcgttatttgtttta, tctcgtgcggagccga,
gtatcatgaatatcac, tgtttaactacaggct, gtaacccagtctaaat, gggctacttcttatac,
gccatcggcaatgcct, aaaggaacagggacgg, acctcaaattctcatt, ttgtgtgcctcttggt,
gagctgaacgttcata, aacaaacgttatcctt, cggtccctgtttttgt, gacagtagatgattag,
gttgcaggagaagagt, aaattctaagcactac, actgtatcccccccca, aattggccggtcgctg,
gtaactatgtggtcaa, gaatgctcttaacagg, ttaggccggagaatcg, tgttagactctttgta,
toccaccccgaaaaaa, cttagatgcaggaaga, actgtggacgggatgg, tcctccttagtgaagt,
cttagagctacgctgc, gtcaggtttgacctct, agcatggtaagtactg, acaccttacactctgt,
gctgtacaatgtagct, ctctcaaagtgggaaa, ggtgctttagaagggt, tgcagcggttgtaaaa,
cttgccgcagcactgg, tttactccttcgtcta, tcacagatacccctga, agggcctgtctgtaag,
cggcggttttttttgg, ggaggtctagtggcca, gactacggtatcctaa, ctgcccgaggcgcctt,
cctcttggggagatac, gattgtgttggtgaaa, gcgtctttgctaggct, ggggaacagcattgca,
aagtgttatccattag, ctctttataaggtcct, taatcagaactatgac, tattgtaccaaggaga,
cggcaatgcctcgatc, tgacgccactccaatc, ttgggggcggcacaca, gcaaccacaaaaggtg,
gcggaagccgcatccc, gcaatatctactctga, tatcttatgcatccaa, ccggtagaggtgagat,
ctcaggcgattgtcct, tgcccggatcaggaat, gttgaattcaaactaa, ttagagataaacaact,
ccagttgcgttatttt, cttttgcactcggaga, atgcctaatcagcctg, ggacagtgggtgtaac,
gcaaaagcctgtaaat, gaaggtgcgtctgccc, ctctctgtacaactag, gtggagctagagcgat,
agctgaagcactagcg, tgggatgcctgtcttc, ttgtgtccccccccct, taaatatgctatagct,
gcatgcggaatggggt, atattagattggatct, ctcttccagttgtatg, gtcagttataagccct,
tcctatccctaactga, gtagttttttgtcttg, ccgcctcggtctccat, gtagctcgaaggaagc,
gacattaggatgaaga, gattctcaggttagcc, actaagatcttaaact, gtgcgatcacctgagt,
ggtgtgtgtttgatta, tcttatagaccttccg, caatgacctaaacgct, gttgctgtcgacctgt,
ctgtaagcataagcat, cacttgtacgatctcg, gcctaatatggattcc, gagccctgaaataacg,
atatagagtttcccac, tccatgtactatgctg, attcttgaaactgtgc, caggacagagtacata,
tgatcttcttagacac, tctaatgggtaaaacc, ggggtctgatcaagtc, gtacggtgattttaac,
tcgtcttgcgggtgag, ataacacctactcctt, ctacctaacataccac, acccaggtttttttta,
ggcttttgagctttca, tagtgtgggagtgttg, ttgttagacgagatct, atgggctgtgagatat,
aaaaaacctcgaacag, aataccagctgataat, cgttgggaataattct, catgtccatatcctgt,
actgggggaagaagta, gccattgtggacggaa, tgcctaatcagcctgt, taaaacctcctggttg,
gcgttttcaccggaaa, gggactacaagactgt, agacgtatagacatac, ccattaggctaccatt,
gggaagcgtttgaagg, tcacgtaaatgtggac, acagttactggactct, tcgtttagtatcttaa,
aagcgcactccaccac, ataaatcttaagccgt, cgtgtgtttacatgcc, aaaggggggcatagta,
gcttctgcctaatatg, tgcgtagaagtgaaaa, gagcgcactgtgcgtg, taggcaataatgggag,
cccttataatatactt, ccgtccggtgctgacg, gactgtgggatacttg, gactcgagaggtccag,
gccaggttctctgtaa, actctttaggtacagg, ccatactttatagata, tgccgttttttttgtg,
cttcttatactaaggg, ggactttacttagggg, aggacatctacttcaa, cctgtgaaaaaatgac,
tgatgaaggttctggc, ggtgtttgaataaagg, gtttatggcacctgtt, tttatgattaaatcgc,
cgcaaagttgtttatg, aggttccaacagaaat, accctctgcccggcac, tgcaccggttttcagg,
ctctgcagaatagtgc, aagtccagtgtatgat, tatcagatagttgact, ggctactggtgcactt,
aaggaaatcacttatg, gagatcatgtcacggg, agtagcccagcagtcc, gttaaatagcaagata,
aggtcactgtctagat, acttagctgattactc, ttaccctcgcttctgc, taaccgcagtgggagc,
tctccagttgcgttat, gcgtcatgttcaggga, ctcgtttccccccccc, gcatcacgttacctta,
tcctgcgaggaggcat, ctgggcttataattag, tttagactgtgacgtg, ggtggagccctgtagt,
cgttggtgcatgttgt, ttagataatccaggcc, cttctactggctcatc, aggtccaacaggagat,
agcaaaaaaaagtcga, gcttcgctgctgtgtc, attacaaacccaccgc, gcggggagggacagta,
atgcagcttctcaatg, tcctttcgcttcccga, tgcttatagctttctg, accttgtgtctagctt,
gatctgagcgttgaga, aaacattacagccatg, atcataacatgattag, gctattctgtgagcac,
tcaaaaaaatgatcta, agactagggcggtttt, tcacttagtgcaacgc, ttccttccattgcccg,
tcttgtgtatcctcct, ggcagggcatagctta, ttttctaggccgaggc, catatgtcgaaaactg,
tagctaatgtctcata, tggtgcaggatcagat, aagtagtattactaaa, cctgtgatgcccaaag,
ggcggcgcggccttgc, attctgacccaataag, cagattccctgaatag, agcctgtgtacaggat,
tgattaaaggtccttt, ttcctgcgtagagata, gcggaagtgctgcgtt, actctggacactacat,
ctgaaagttcggaata, ccaccatgcaatgatc, cacgatgaacgaaaat, gggggcattatggtct,
tagcaccacaaacatc, gtcatatgaagtcctg, tcccaaatacaaagag, ccctctggactgatgc,
gtccccccacccgaaa, tgtctgaaaatccaga, tgggattgctaagaat, tgcacttagtcagttc,
atccccccccgaaaaa, agtgatcatgggcatg, cagaccccgagaacta, gagacgcgggcgggtt,
tgcctccacttgttca, atgcttgcaactaata, caaggtctggtttaag, ctagggaacatggtta,
actatttgctcttgtc, ctgaccaccacttgat, gtgatacagatctgca, atggaaaatagcgaaa,
ctgaagcaatcgtccc, catttccggtaaacta, atggcccaatcttgtc, ccatgcccccgaaaaa,
ttactttacgtgttta, ggccggccttgcttta, gtggtcttacttaggt, tagcactcgtgtacta,
gtgagagtcactgtgt, aactgattctcattcc, gcttagaggtaatgag, gaactgtcaaacattg,
gcgggggggcccctgc, tgattgtgggacgttg, gctacgaatgactttt, ttactggtgtaaggta,
ttagctatgataccac, acccgtgatcttttta, tttctggctgtatgcg, tatttcatcgtattct,
gtctcactacccttag, ggccgcgttgcggcga, cttagtagctggtgct, gtcctttacactagac,
agaccctgtgtagggg, agctgaaactcatacc, gcgtgtcagagcctgc, ggtgaccatgtggcat,
gcccccccccatcacc, cccgaggcagatctat, cacacggcacccattt, atattgggtaaatcat,
tggagtactaatcctg, caaggacatatgagca, cacttccagaaagacg, tctatgaggtccacat,
aaaaaactggtctcca, gttgtaaggaggcagg, tcaatagaccactttt, cagcccagtggtaaga,
caagatgaaccttagc, atgtagattcattgta, ggtaatggtgcggtgt, cccactagaaccaaaa,
cgcattctcctgagta, taaggagtcctgttca, tatgctagaagggtta, accgatgatgcagaaa,
gtttttttaggggggc, gggggggattcaaata, tgcccccaactcttac, agctccctgtatgcgc,
ggaagtgctgcgttgc, ttttctggcaccaacc, aggcttaacaatttgc, taaggtgatccacgtg,
tctgagatggatatct, ctcgtgcggagccgaa, gggggggaggcttttt, tcaatcttgggggggg,
gacgtcgaaggcaggt, cttggtctggccgggg, accaaggacaatggcc, aaaatcatgtaccagc,
aagaagctatcgatta, gtgccgagcctctgaa, tagtggatttggattc, gtgccgcaccaattaa,
cggtctcccccttatc, gtgacgttaagtcctc, agccatatgtcgaaaa, ttcttaggcctggcag,
ggttagaggctatctt, ccctatataagtgaga, caccgtgagattatat, aaggtaatgtcttaac,
tcctgacattagctgc, cagccgtcgtcctcga, cgagcagtttggaagg, attggtttcctgttct,
tctttttttcgatgcc, gagcatttacctagtc, ttcctaccaggatgca, cataatttagtgttgg,
gttatcctgggaggcg, agttacttaagactat, tgacagaggattaagc, gtgggttattttgagt,
ggtagccttactctgg, ctagcctggaaaaggt, tgctctcatacccttc, cagccccccataatca,
attgttcagagagtga, gacctgggagatggtc, gctcaccagtgcactg, ccctcttttcccgctt,
aaagataaatctgcta, agctcggttaatacgg, catatttatggaggag, aagtgtgggggggtca,
gcaaacctatttcgat, atcaggaccacttgct, gataacactatgtgat, ttatcattacgaatat,
atatttacgtattaac, tccagcaaatggcggt, agactcctctcggggg, aatcaaaaaaaagggc,
cctgatgaaggagtgc, tacttatgactctcgg, ccgcttagcgcagctg, aaaataaagcggactg,
ggtacaataactcagc, tcagccggatgtgggt, tacagaacagggtaaa, ggtggctcccttccgt,
aggtagagttgtctca, aaacgttatttagagt, caatcacgtattttct, gagttccgctcttgta,
tagtttactctactga, tacgattcttgagcag, agcccagtattcctgg, ggtttttcccccaatg,
cctacccctgagttcc, actctgtgccccccga, gtacggaactcatttt, aggaaaagcgtagcga,
gcctataccctttttt, ctagccctgttccttt, tgacttgtttatgtgt, agaggggtaaggtagc,
tgaccctgatgcccgt, atatatacgatgattt, ggcttgttagttaatc, cacccttggtggcaca,
ccagtgccggaacagt, cgttgttgttaataat, aaacaaatttaggggt, cagctatgtgccctcc,
tcggaagggaacagct, cagttccctgtagaga, caaaacagttagactc, ctctgctcccacgtta,
tgacgaggtcctgctt, ggaaacgcggggtgga, agctgcggcctgcgca, cagacctgagctttat,
tttccctgtagtgtag, cttccctacattgctg, agcccggaggcccttc, acccttttttatgatt,
ttcgggggggggactt, tgtttcaaagagcacg, tagcagcattagaact, ctttttgacactggcc,
tcttctgcttcgtgta, tggagtatcttcgtgg, ggaaaccttgcatcaa, atcctaggctccctat,
aagtgaccttggttct, cagggagggtaacata, tgtgcccttgccttgc, aaggagatacccatgt,
agtcttaacagtgtgt, ccatagccagttaact, aggcaatactagatca, ctcttctattctagct,
caatgggggggggaga, tcaggacccacatgac, aaacgggggaaaagga, aaaaagtcgtacctat,
cacatatcaatcagga, tggccgtagcttagtg, gtgggcacgtttgaca, catcatactggccatc,
actctccaacatccgt, atgaaggattaatcac, gccctccaggtccgtc, gctgtacacattcccg,
tgtctgcatgcgtgtt, cctaaacgcttcacag, tataatgataacgcta, cacgtttacctcttgt,
caataacatgtactta, tggggggggacttttg, cgaggtgggagcttca, agtcattctagtcttt,
aggtttttccaggtct, catcagatgatgccgg, aaaaagtgcttacgca, atcctagtatatgtct,
atgcagatggcacttg, tgagagttaccgtaag, catgccctccaactag, gcccttttttttagta,
ggacgctcccatctcc, gataagagagtatgat, caggttaagccattta, tcctgtttaggatcga,
gatgcccccccccagt, aagatgattgggggga, gacatggttctatctg, gccttttgattatcta,
aaaccatttttccccg, acataggacacaatag, aggaagatcgttttaa, cgtgttgtacacagtg,
ctgccagaagggttaa, ttaatccagttaactc, ccctttaaatctgaga, atccgccgctgttgag,
aaattatgctagatgc, acgacttgtgacaact, aaacggggaataaaag, aatggtcccccccgaa,
aaaattgattgagaga, caggcgttaaccacct, atgcctcattatactg, gaagggggggggtcct,
gcaagttattaggggg, gtagtcctacgttagt, gttgccttcccgctta, ttaatgctccttattc,
agcggtgagtcatcta, gaattgataaagcgga, tcttctttttcacgac, ggagggtgttgtttgc,
ttattccctacggtat, ggcaaacattcacaag, tggatgccgaggtcac, ttttagggggggtgaa,
ggataaatttgtaggg, tgctagcattgtatct, gtcttatctgttaacc, aagactgtgcacattg,
gacgaatctctgagct, gttgctgagtgcggtg, ccaattatatggggta, gcattccgcgctccgc,
tgctctcccttagcaa, ttagttgagtgattgg, gtgcgtctgccccaca, acccagtgttatcctc,
aacaggttgattactc, cctcccaccagtaaac, gttggtaacttacaat, acatctggatgcactc,
gaggtgctaagtcctc, gatgaagaacttatgt, actgcttacattccta, catttcccatatgggg,
tagacaataactgatc, gcaaatggacaagaac, ttgctgttcctgctct, tcgtttccccccccca,
tttcgagacttctttg, aaaagttgctctgccg, tctgtaactgtgagtg, cataagtcggctggcc,
gttgctttcactctcg, attagacagatcagaa, ttactaacgtaatggg, ttcgtcctctctattt,
acagaaggtcatggtc, agataccatactagat, cgttccaccacccctg, cacccccccattcctt,
gagcctcccacggccg, cctgtaagatcatccc, gtcctctgaacagtat, cttttgtggtcttggc,
gtatatgccttgtcag, catcccaacagtgctc, agattgactctgagac, ctattgcgattgaagc,
ggtgtttcttatccaa, ggagcattagccagga, tattccataacaacag, gccgcatgggcttctg,
cttccgtgctgtggtg, tatccgccccccccca, ctaatcgactttttac, tcccgcattcatgctc,
tgcagacagtgactgg, aagctactcaataggc, ttagatcctgttccag, gcattcctatcataaa,
gtataaactcacattg, ggaaaaaatctccaat, tcgtccttcaccttct, tgtacagatatcacag,
aagttaagttaacctg, gtgtgaggggggtact, cgattgaagcagacat, gtggccctccaaggca,
ctttgagatgaggggt, accaccgggaaggtca, tctatgagcagattaa, gatcattaacacatct,
agctagtgaaatctct, ctgcctctttatcaca, caaatggcggtgaagt, atgggggggggagaag,
cttccgctcgcggcct, cgtgccacgatggtcg, tcttcgtcgggacgtc, acgcatctgatgaaac,
agtgcttgataagaca, tgtatggctaaggctt, ggctggatccagtaaa, ggtgagttccacccat,
agcatgcccttaatat, atccaagccctgtgag, agtgcctaaataaaca, ttgggtaacctgttca,
tgggcccgcaggaatg, accaggatgtgtattg, tgcgtgtacctcccac, ggggggtcagaggttg,
aataatgatgggctgt, agctgcgtgtgggtgg, cagatacctttggtct, cctagtagttggaata,
atctgccctcgccccg, tatgatgacgggcatt, ggcgtaggataaaatg, tgattgaactacctcc,
tgtcgcaggctccata, ctcattcagatattag, cactatccaattatga, agtccatgggcaacgc,
acagccccacttggat, gcggaacgtgggggtc, aattggtacgaaggtg, gagtcttgttttgcaa,
ttgtctatgtggttga, ctgtaccacgatttct, ttgtcggtggaaagtc, atgtgaaaaaatgcgc,
actcgtcttgcgggtg, atggatatacatatga, gcttaaattgaataca, atctcctcgagtttaa,
gtaactgttttttaca, tgcgccaactgcatgt, agatagcaagttcact, agcaggtccacccgcc,
tcaattagactgccaa, tggggaggacgaaaat, aaaagtaccccccctt, actaggtgtgctcatc,
gcgagggctgcctcta, ggcatggggtgagtgc, aggcgtgctccagcac, tgtataactctctctt,
gttttttagggggggg, ggttgtaatcctattc, gcccttttttttgtca, gccttgcgagggcagc,
tacctgttcaactaat, ggaagagtgcaactcc, aggttggggggggatg, gttataaaagaatcac,
ttagtttgcctttctg, actaaaaaaaacgtcc, gtgtcaaacagagagt, cacaatagttaagagt,
aggtggggggtaagat, gcactcgtgtactatc, atgaggtccctatgag, aatgatacgctctaag,
ctgaaacgccacaagc, ctaattccaattactt, atgcatttgcagctgg, tgactggactcgagag,
tccacccgcccctaca, tgctgggctagcaatt, attgtggtggcatcat, catccatgcccccgaa,
tggacgccccccccca, cttggcgacagagtta, gcggccatgcttcgct, agaaaccctttttgtc,
tccagcagcggcggga, ccaaaggcactctggc, ccctcctttttagacc, tatcgaaccccccccc,
atggttctacacagtt, caatatacaccgggcc, tgggttactggctgcc, gtctccttcgaatgtc,
acagacgtgatcccaa, agcctgggttgcttaa, ggaatgaacatgggtc, ttatctaccctaactt,
aattgaagtcgtttta, gtcagcggaggtgctc, atcacgggggggggga, gagctcttaagtttgg,
gagcacgtttcatcta, tccttgaaagtcttgt, aatagctattcacaca, tagaaatccataagta,
tacaactccccaaagg, ggaagatcgttttaaa, gtatgtcaactttagg, ttggtgttctcctata,
agaatcaagtccctga, ggattacttgaattcc, cgacacagttgctgaa, cagggtcgggggccgt,
tcgcaaccttcccctg, tcttaattgcgatgtt, actgaagatggtgtgt, aaccgtttgagacctt,
tgcggctgtatgtaga, ccgcggcacctggccc, aggcaccaccatttat, ctataataggcctatt,
cctgacgccctctccg, ccagcacacattgggt, aaactatcgatggata, caagcgttaaactctg,
gcgggcctgacttatg, atcccgggagagtccg, caggttgtaatttggt, tcatgattcgactccg,
ccctcgctctctttcg, agcagtattgtgccta, acaaggcataatatcc, cctgttcccgcccgtg,
ctcatagggaaggcct, acactggtcattggag, ccttaagacatggaat, gcttcctggcagatac,
cttgaccaggtgccgt, atgtggaacaggtgca, gtgtttcacccattaa, ttagtctccatacccg,
ctctggtgggggggaa, tgaagacaccttagtt, acgaaaaaaaaagaac, tgtcgggacccctgag,
tctgctggaggccgcg, attcactaaactccca, ggtgaaaaaaacgaga, atcttatgctgattaa,
cgccattaaaaaaact, catcgattttcttttc, ttctaacccccaagtc, cctgatccttaatgct,
gactacagagcctggt, aggctgttggactacc, aattgttcaacagagt, tcaatgcttagagcta,
agaacctatctagcct, ttatactgggcattaa, cacctgccgtgccgga, tagtataaatccttta,
gttgattatgtgcttc, ggactacatgcgttca, ggaggccgcgcagaaa, tgactccgtgtctgac,
aattagcttctgtgag, gacctgagatagggga, tgaacatccttagggc, ctcggtttttttaagg,
ttcatgacgatgaaaa, aggcaccctccacgac, ggctggtatgtaactc, gattacacccccccgc,
attatctaagagagtt, ttatgggggggggcga, ttaaaacctcatgcag, tgcattgtcagtaagt,
ccaccgggaaggtcag, gcaccctccacgacgc, ttcttgcgtcttcgta, gccgcttgctagcaag,
tagggaaatgaggacg, tagactagttctatac, gtcctggagtagtcta, gactgtcgtcatggct,
gggcctgtcagtagat, agtatgaggggtaaaa, ctcagattgcctctag, cctccagcttgcatat,
ctttaccccaatacga, agagctcctttgtttg, caagagttatgttact, ttgtaggcatgtagtg,
tgcgctttttttatta, tgctctcattatcaag, tgccattgtggacgga, cggctggacattgacg,
gattatgcaaggcgga, tccatttagatacgcc, ggcattttactggatg, agtgatgcgagattgc,
caaccttctcgtttgg, tctgatactctcagga, aggcgttaaccacctc, gttttcacacccattt,
ttactagactactttt, ctcggtcacacatgat, ctgtactgatattaac, atacacatgggggttc,
ttagcttcaccctgtc, ctagaaaccgtgcagt, ataatatctacccaag, ggctgactaatgtgtc,
cactcaagtctactac, cctgatattcttagta, ggctgctcgcagcgct, gttcatctggaaccga,
cctcgtttataaaaaa, tttcttccgtacaatg, agcccgggggggggga, ctcagaaattccttac,
tgttagccaatgcacc, atcttcccggtctttg, tttagtccctgtattg, ggctccgcaggcatgc,
ccacggacctttgcgt, cccgggtcttaccccc, atataagacgctcaaa, acataggagggatttt,
ctgtatacattgctag, aaccttagcattcaac, atcttggggggggttg, cagtgagattatatct,
ttactctgaagggttg, atgcatggcgggctgc, aagcctatttgatatc, gactgtgcacttcgtc,
gcaacctacgtgaaag, aaggttgtttgagcca, caatttacttttaggc, tcccctcatgtaccca,
agatggggtttaacgt, gggaagtgcaacacca, cagcccggaggccctt, ccccagtttttttgac,
acctactacgtccttt, tcagcgactcaacaca, ctgcagccgtgaggag, cctgaccagcatggtt,
ttgtaatctgagatcc, ggcggccttgccgcgc, agagtttaaaatcgaa, ccttgtaggaagggac,
attaatccacctttac, gtggaacgtccgtcag, cgtgagacctgtgcca, tgagcaccactccaag,
agggaagcgtttgaag, accttaggggtatatt, ctcctcctatgggtct, cactgttgcatgatga,
ggatcatcagaatgat, ggagggaaccgaagtc, ggactgggtgagtaag, tcccatccgttgcatg,
aaattggagaatcggg, tacagagagattaggg, agataagaagatttag, tcccgccttcctgttc,
tgagcgcactgtgcgt, gatagcaagttcacta, attgctcccccccggc, aatcagttatgtggtt,
gatagatccttctctt, gataacagtgactaga, gatattttttttacta, gggggtatcatggagg,
tttctccccccctgta, tattgcccctgtcctg, gcatcagttgcttaca, gttcttactaggcagc,
ggatttcagactgaac, actggtgatgtgtcca, aggagatcctgctcgc, gttttcccccccaatt,
tgtactgctctcacca, tagtccaaaaaaccct, gcaagtgctacccctg, gcccgtcttctgtcct,
gattttctgcttagtc, aaggcagattagcctt, tgtaaggcttaatagt, ggcttttgaatagtag,
ggaatagtctgacctg, ggcttaagcgggaaga, accttctcgtttggcc, atgttagccacaattg,
cttgagcccgataggc, aaccccccctggcctc, tcggtggaagaggagt, tgggaagagttatgct,
actcattttcaagctc, caagtctaaaacttac, gaccgcaggtctcagg, ccagcatagttaaacc,
ctaaaaaagctgccct, cactctgagatgcttg, atgcattagcgaacac, ccaattagaccaagcc,
aacctccgcatggtgc, ggcggtccttccgggg, taaggaatcaccgtaa, tccacgtgaaagataa,
ggaggtagaggtttaa, aaagtgtgggcttcaa, cctggtatggtagtga, ggcgcaccgtgagatt,
ttataaccacaaactt, ctagacttccttgagc, taacctcaatactgag, tttatgcaggtgaaca,
ttggagccttcagtga, gaaatgtcattgcggg, agcaagagtggcataa, cgcgggggggcctttg,
caatccctatgtgtcg, gggagctaagcggtga, atacaacgcgcccccc, cctactccccccccgg,
agccccgatgcagggg, aaatacggtcattatg, aaatcggttttcaaat, ggtgggaccttcgggc,
tcatgctccaatgttc, gcgcttgtctaagcct, gagcagcgtctttgct, gctttacccatcacag,
ggctaacccctgcttc, ctcctaggtggtgtgt, ccgttactgaggtgct, ttcttcgttccctgac,
ccggatattgcattat, gggggggggtaggaac, ggaaccacagcttata, atagaatgtcttaagg,
gagaggcagagtccgt, cacgagcttaagcagg, ccctgtagactgggac, ccgtcagcgtgtggaa,
ttaacctagtcattgc, gtactcccttattagt, atcctaataatcttcc, cctgaaggccttacaa,
tcgtgtctccatggga, cgccagcgctcgcgcc, cacatcttctagtcct, taagcacatcagctta,
gcaaggggggatatct, gacgcggggtggttga, gtgggagtattacaga, ctggcagaggcattac,
aacacacactgaagcg, ctgccccgaaaacaaa, tgatgagttcagggca, tcttttctgcttaccg,
tctaaccttccaggct, tcttacacacagggcc, gagcccccccctcccg, gtgtgctaccaatttg,
acacatcagtttactg, gtttgaatgatagatc, agctaaaaaaagctta, gggactacatacacct,
tgtgcgtgcgccgcgg, gtgggtgcgtgcatct, gcctttggcccggtgg, aaaagggtccccccct,
cgtttcctacaggagc, tgtgaaagctctcatg, cgtggggcctgtcagg, acctgaaataacaccc,
cgcactgagcccctca, agcccaacataaatct, gggctgataatgaggt, tgaaattcccgacctc,
gttctctcctttggat, gcatgaactgagtggc, tctcagggacttttgg, ccaagaccgactcatc,
cagggacaccccccca, aggcccggccttttaa, ccatcgccttagactt, tacttagggaagagga,
ggtacgttaggtaaaa, gttagcaccattcaac, aggaccaccaaattga, tggtgattcgattttg,
ggaaccccccacgcac, gcgcacaatacgcccg, tgttctcactgaaagg, atacctatgtattgat,
gggttggtcactaact, caatgacgaagttttc, gacgttacatgcatga, gcgtgtggatgggctg,
gctcagcttaagcagg, atgacctccccatagt, cactgtcggctattgg, tcatttgcatagacct,
tagctccctgtatgcg, acgtgtcaaaattaag, agatggtattgtactt, ccccattgccttgttc,
gaaccccaattgtttc, aggtccgtcactgact, gggaaatcttagagcc, taatcgactttttaca,
ccatgccacatgttag, acttaacgccacatga, atggggataacggaac, ttgctgacggaagaaa,
tgatttcacggattag, ttccgatgacaatttt, ccctaacttgctcatc, tcccccccaaaaggct,
ggttagggtctccaaa, cagccaagagtggact, ttcagtaggaaaaccg, atcaagcacagtcccc,
gccgagttattagggg, gtgtcccgggacgggt, ggagatagaaacgtcc, gagagggcgctagggc,
tgatgtttgtactgtc, gacagacgtctggaac, accagtgcactggtct, tctgtagcttagagcg,
gggggtggcaaaaaag, ataggtcaaggatggt, ttgattggtgttcctt, ctgtatcacccattaa,
ccacttggatccaggg, caacccctgtgacact, ttacaatgcggatacc, tctgggctttaagttc,
cactgaagatgctcgt, gccgaggccgtagggt, acggcaaaaaaaacta, ggatctaccttgtagg,
ttattttatattgcgg, cagtcgttgcttatga, tggctggtgtgccgag, gccatcacccagcatg,
gtctgtccctagagga, cccctttgtcagggca, gcgttatctcagctga, ttagttgcaacttgcc,
gggctcaaaacgtagc, gcagtccttttttaac, tccaggtccgtcactg, taacattggttgtggt,
ttgtaacttagatctt, ctacaccgcagtaacg, ggggggcatttatcat, gtctttcaagggtttg,
ccttgttgttgccact, catggagattatcatg, taccgtaagctcggcg, attgcctcctgtggct,
ccagcctgcacgaggg, tgcttacaagctggat, aggtccgtctcgcctt, tggtaagtgtctgatt,
gatcatgataatacca, ccactaacaggatctg, aacacgaggttttgcc, aatgtctagggaggcc,
ccgtgttacataggct, gaataaagcaaccaca, gaatcttaagctgaaa, gatctaaacgcgctgc,
aaaacggggggttcag, ctctttgtgtatactg, caatcttggggggggt, actcattaatgtcgca,
atagcgtagctcgaag, gtgtttatggctattg, ccccgagaactatgct, tgaagcttagagctag,
atgcccccccctccct, gagactgagctctcgt, cccattaggctaccat, cattgtaggtgtaagc,
cctaggggcaccacga, ttatctcattagccac, gcgagatcttgcataa, agattggccgggctcg,
cacaaagagcttgcgg, ggctccagtgttttta, cagtcttttttacagg, gctcagtgacgttgag,
ccagtcgctttttccc, cggaaaaaaaataagt, ctttagttgctccatc, tgttattgggaagctg,
gcggtggtacctgttc, ccgcttggctgttcct, cgttactttttcctgt, cagggcatagcttaac,
cggacctttgcgttga, ctggttgtgatattcc, ctctgaccgatttaag, gcccggctgttcttta,
aacgttttctattata, gtggttagccctgatc, gtcgggaggggttcag, atactccaatgtgtgg,
ctgcctgttatactga, ctccgtcacacaaaga, gaacgtgactagtgtt, cggcattttgaccttt,
ttaggggggggagggg, aaattgcggcacaaag, ggcccacaggcaacgt, aggctacccttatggt,
gtcctcccaaattgta, ccccagttctactaac, agggcggctgtttatg, gattttccacgccctg,
ctcgcacagcaataca, cactgtgttatcgagg, caggtttaaccaaccg, ccgcccctaaacccca,
tggctcaggggtagca, cttcgaatgtccattt, aaattccatccccgaa, tccggaaaaatacaag,
ccgtctgtgacaccac, ggaagctggagatatc, tataggtagagcttag, tgtcctcttgtaattt,
aaagaccctctaccct, cctaaaaaagagaact, acgtcaaaagtagaca, cttgacagtccacggc,
catagggggggtcagt, ccaagccttgatggtc, ggcctatcaatttggc, ttaatcaggtatatgg,
gaagaaggagaacgca, agcccttctgggctac, agaagcaggctctgtt, ggtcttgaaggttgat,
gctatggtctagctat, ctgcttgcgagttttg, tctgagagtggtttca, tctacactcgagagac,
gaggccgcgcagaaag, attggccgacagaaga, gcattagcaatgcaga, agcacagcccctgtcg,
tgagggggggcagggt, gcaagtatcccaagcc, cttcctcatctatgtc, aggcctaagtaatctt,
ttgcgagggcagccgg, ccatgcagagacttgc, atggcccattagggca, tctaaccccccggtgt,
aataccaacactgcta, aagcaaacctatttcg, cttgagaaatcgagca, actaggccgggcttgt,
ccaccctgccgacctt, gggtttaaccttggtc, ttgcaaaaaaatagcg, aatgggggggaagacc,
gttagttgcttcacct, cgtccccgtgggtatg, cggtccgatctgccct, cacaccaaacttgtct,
gaattgaatactgcta, ggactacataggtaaa, tctgcagcttaggcat, tacactacaggagact,
ggagacgcggggtggt, agtaaaacttctcgtt, ctcgcccttccgcact, aatccagcacatatgg,
ttttaaaacgaccgat, catagatgcaagataa, tctaatcggccatttt, cctgctatcttacata,
gtgatactttagaatg, tggatgtcatattaca, cataccttaataagga, acaggatctcgaaaat,
attagcgtaatggcaa, gacgctaccggccttg, caataacttggtgggg, acaaagagcttgcgga,
aagcgccttggtcaaa, tgttgtgtcgttttct, gtgagttccacccatt, ctaatatgggggggat,
ctagttcacagatacc, ggcaagtatgtcccca, gattattgtaccaagg, caggtaccactgacgg,
catccgccgctgttga, ggttgcttcttccgcc, acccacttacttaaac, agtggtagctatacat,
ggccaaacttaggact, atgttccttctattag, ggatgcactaaaggaa, cttagtggaagtaaac,
tacaacttttgcattc, taatagactttctgtc, acggtaataaatagta, tgatactgtagcaatc,
ctaatatcctgagggg, taacacgtgagaatgg, atccaataccccactc, cctattcagccatgtg,
tacttagctcccacat, ctcatcctcacagcgc, tgctgtcgacctgtac, cattagagcaaccatg,
aaaaaatgtaatagcg, aagacaagctacagcg, cactgatcgataatat, tgactatgtccagcta,
atgaaggagcatcatg, ctttcagacgccttgc, gaaggagtttgcatgt, ccagagttgcacccgt,
gtaggtaaaaaagatt, gccttcccgcttaggc, cgttgtttgacatttg, ctgcattcttaactaa,
tttgatgcctgtttgg, ccacaataccaagcga, gtactcttttaattcc, tgggaaaggcccggcg,
cccgggggggcagaag, cttgctagttcattag, cattaaccactgacct, taatactcaggttaga,
ttatgaactgaactcc, gttggtcccagaagct, ctctggaaatagccag, gctggccaaggaacct,
gtggcgatttttttgg, ccaatttaagtgaacc, aatttgcgcagaaatt, cttgtcggtggaaagt,
atctgatgcaccagag, ttcctcacctttatca, tgcataaagatctcct, actagggcggttttat,
ttagtgtgttctgacc, ccctcgctggctctta, aacctcgaacagccaa, ggcattaccagttata,
catggatcattactca, gaatttcgttgggggg, ctacataagtctgcca, agttgtgagttacacc,
acgattcttgagcagg, gcaggcggatcgcctt, taagtcacccaatcct, agaactcatgaaccac,
aatatgatagaagggg, gcggtggtcgagtcct, gaagccccgatagaac, accgcatgacgaggtc,
caacattttgacatag, ttgccacttgcaacta, ttctgagttcgtaatt, tgcttgaacgtcctca,
gtgattattgtgggat, tgcccgaggcgccttc, aaagacctccccccga, gactttggtccaatct,
aacaagaaaacgccat, gatttattctaatgct, aggtatcagagtgaaa, gaaaggaggcatatca,
gatgcggcctggcgcg, tgtcatgctctcaaag, gcaaaaaagtgcatgc, ggtcaaatacacacgt,
cacctattttattagg, acagggttactgggtt, tttattggatcgtctt, atgtatcctcgaaata,
ggggcatgtcctgata, acttgttggcttaggc, tgcagtgtcatgtaca, tcccgcccttgcgccc,
ccgaggtcccagagtc, ttcgccataattaagt, ctgaggcgaggtgatt, gatcaggcacacttta,
tgctcccccccaacaa, tcgttatacttaagtt, tccaccegcggtgcct, cctggctcagcgtatc,
gtggtcaagctgggcg, tgaagtattagagacc, ccatgacaagatgaac, cggggatgaccgtagt,
gccctcatcatcagca, aatacaccagttcacc, ctgatcccagtcgtgg, ggaaggattgagccgg,
ccaccacattaccaga, ccgatgcttaggagca, tacttgtaacattcag, acagctacgttgacct,
acctgttctccagttg, ctctatacctgttttt, agtctcgaacaaagaa, tcctgtaagatcatcc,
gcgtacatatttgggc, gactacaagactgtcc, gggcatttacatgccc, tgaagcgttttttttg,
tgggagtccttatcca, tcagttataagccctg, cacccaagtgtgaagt, ccactccgttccaact,
gggctgtatgtacagg, gtcctttattagttaa, gttgcttcccccccca, caagttatacccttcc,
gcccatttggaacagc, acaagctaaaaaaagc, agttaatgtgggggtg, acaaacggccaaattt,
ggtggtgaagctaagc, cttgtgctggctcgcg, gactctgatgtgtata, accaacagatcttgtt,
gcgttttttttttgta, tgctgcccctaagggg, acccgagcactttgcg, tctgaactgaagttag,
cacggagagcatgccg, aggctccgaagtttct, aaaaatggggggggac, atgtgtcatggctgga,
ttgcgatcaatttagt, gtaaaaccgaactttt, gcctcatacatgtctt, tttaacgtttgtaccc,
ccgttttgagtctgaa, tatgttgactttagga, ttcaaatacaagcgga, tacgtaccatgatgtt,
tcagggatcgtctggg, gaattaacctcatact, ggaaaagtgcattaga, cagcccttaggtgatg,
tagaatcttatgagtt, acaagcttaaatcagt, agtacattgatactgc, ggggcagagctagatc,
ccccgttgtcccttag, gggcctcggcctttct, cttctaggtacccttt, ctgagtacgaatgcta,
agacaaaaaaagcgta, agtgaattcactctac, ataaatttgtgcttac, aaagtgtgccccccca,
cgagtggaaaaaaaat, ccttagacaaaattgc, ttactcaatgtacaag, atgaccccccttcctc,
gatgtaagcctgtctt, ggcgagtccaacgttt, atgccccccccccatg, tcctactattacccat,
gcaggccttgcttgta, cctctcctccgtttgc, gcttgaatctccccga, gaaggaatgttcccta,
gcggggatcttcagag, tacacaatattatccc, gcggtgagtcatctag, agtaacttgcagggag,
ggagtggtgattactc, caggccttgatggaac, gtatcactatcagctt, ttggtcatactaatcc,
gggcttaaagcccttc, tttactggcagatatc, gaccccctaagggggt, cctgtgttgaagttcc,
gtaaatgactcgttaa, gtttgttcctcgcgat, ggcttagaagccctaa, tgccggcttagaggaa,
cacccttttacagtgg, agtgccataaccaata, gcttagaagctaaaca, gacattaactaatcat,
actccagttgtacaca, gtcctgtagcacttgg, acatgacaacctgccc, cccgctcagcgccagc,
caccaactttataggc, tattccctacggtatc, tccggcatgtagaaat, ggggctaggctggaga,
ccacatccctctggta, gggcgcattgcgccgg, gccccacatgtcgctg, acgtgagcatacagga,
tcaaattaatatacga, tataccagttccacgt, gaaggctccccccccc, atcccatgagtcctca,
gccctgaaagtccaaa, caatgcggatacctta, gttcctcgcgatacat, cccggtgggcgaaggc,
gtgtgcctaaggatgg, tcatttccggaatggt, ccaaatggcctcctca, tcatataggattggtg,
ttcccactgacactcc, gctcagacagaatagg, aatggcatctgaacca, ctcgaagcagtcaaga,
ccgccccccgccagcg, gctgttagtcacagct, cccctatctgggaaga, gaaagattgctctgag,
tgcttgcgagttttgg, cctacctaaagtttca, gaagggcactgttgta, gccactggccaggtta,
gtagaagtcaatctgc, tctgtctgtttatggc, ctcccacgctcccaga, ggcaacccagttagac,
acagtgggcctatcaa, tgtgaggggggagcgg, acagtcttgaaattag, tccttagtatgcttaa,
gggggctttgccccca, aagaagaaatcctcgg, cttgttgaagatatag, cctggcaatgcggtaa,
gcctagtatgctgtca, acttgaacccccccca, ggtgggctctcccctt, tatgctggaaagggta,
tataaagcgcttggct, tgatgcctgactccta, attcacttagagctac, gagttattgtccactc,
cccccggccttttccc, caaaccccagtgtatt, ctacccaactcctttc, tctatcttagtcttaa,
ctatctgacaagatat, attcaccagcgcttca, gccgctcggaaatcat, ggccctttaaatgccc,
tagcactaaatgtaag, gaaggattgagccggc, aaatcttttgaaccag, ctcacattggcctcgc,
tcattttttttcccgc, ccaatacctatccacc, gagccttgggctgcgg, gacaccttacactctg,
acaatttcatatagat, tgcaatgacctaaacg, acactcttgtggattt, taaagaggggacactc,
gcactctaaaacatcc, tcttcaccaacctaat, agctaccacgtgcttc, ataagacacgtgcatc,
tcatccccctgtgaag, cctggcaactatgcta, cctagacaggtgcatg, tggattggatgttccc,
agcaaattgagcagtc, gtgatcccaatatgtg, gcacctccaaactgct, tcaaaaaataaggacc,
ccacatcagtttcagc, ttatgactctcggggt, agatagctaatgtctc, ggtgccgttgcctgtg,
acccttcatcccatac, tcttcacacgggacct, tgttttttttgcctac, gctgaaaaccgcttaa,
ggccaggtggtaggat, actctcactgtgaaga, gatgaaggttccataa, ggcttagtagaattcc,
tgctgtttcaatcctg, ttgcttcagtgacagt, taccctgctccataac, ggtgccggaaaattct,
acataggtgagtctta, cttttaccctggttac, ggaacccccccttcac, aacgttactaatggag,
agaccagtaaatatta, gcaggttatgtcacgc, ccgggcgcactattgc, gaggtccagacctgaa,
ttttttctagcacgat, ggcccttttttttatg, tcccactttgggtcac, aacacttcccccccac,
ttgtagaagtgttaac, aatagaaggacagggt, tacgtttgtggattgg, tggtgaagctttacct,
caattagcgcctggga, gtaaataaaccagacg, tgagtgagctccaccc, tgtccccttgcttctc,
aggagagatgccgggt, atccttcacccatggc, cacaaccttccccggt, tatattcatgctgtcg,
acctgggggggtcatc, caagctataggggcct, agtttgagtccggaag, caaggttccccccctt,
aattaggccgggagct, acatggagattgggat, aagattaaccttacac, tcccaagattagatca,
ctgcacttaggtggaa, gtttgctccttatcca, accttagctgttctaa, cggtcttcagaacctc,
tcatcatgtcagcgat, taaagggctttccact, taaagctgccctctcc, cgtgctgcgctgtcaa,
cggttgaaaaaaaaag, agataagagcccccag, catggacacttcgtcc, tcggctctcacacggt,
gacttatcagactcta, gggcgccagagttaga, gtgaagccccgataga, tgaccgcgttagccag,
ggctgtgagccagata, gattcttaagtgacta, cctctgtctggtactc, gcgatcacctgagtaa,
ctacttactgaaagtg, gagcagatccttagat, taaattatacaatacg, aatgcccacccctaat,
gccagtgtcgccgctc, gggttcttgtgtgtat, gacgcaacaacaattt, tagtttctgctccatg,
ccctaggtttgtgaga, acccttctgttacatt, ctagcttccaaagtgg, agagcttaaaaccagt,
cctaaccgtttttttg, tctcccagcggcaggt, tgtttatggcacctgt, tacttttaggccagca,
ggtagcagtgaacgaa, gaccgtttggacagaa, cacccaaagaggctat, atgccgcctgccagtt,
cctcacaaagtggtct, agctgaaacgagaatg, tttaataggggttagt, acttgtcttattgtta,
tcaccttttttagccg, agtacttatgactctc, aaaaaaagcgtagatg, aatgagataggtctcc,
agaccttttttttagg, aaccaactgtgggaaa, attttggctggggcga, attttcttgttagggt,
gagactgtgtaatcag, tgattgacaccccccg, ctagagccacctgcac, agatgagagtgttctc,
tggccccttatcttaa, agaatacgtttgtgga, gtccataagaccttaa, tgctgtaacattcttc,
atctagtttttttgtc, ggagttctcgcgtgat, ctgtttctgctaagca, tcttgcgtggtgctcc,
ttcccggggggtgcac, ccaaatttcatgctcg, actcaacccattgtgc, tttccagtgagtgaga,
cggttcctggctgttc, tttgctgcgcagctgt, gaggctcttagttgga, ctcgaaggaagccccc,
ctctacgctctcatcc, tattgtgccttaaagg, ttatgtgcagtcaggc, agatctgaggccatct,
agataagggggggatg, ctcagctgaaggcata, aaattcgattaaagta, cacttctttgtcatgc,
agaagtaaggcaccac, ccgactccggggcaaa, atcttttacgaaagcg, aaacttcctctatcca,
aactgaattatatccc, tttacgcccaaaactt, ggagctgtgtacctga, agttaggtctttctca,
caatgagacaccttaa, tggggtacccttttga, aaattcagcggtgcgg, aaaatgtctgcggcct,
tctccttcgaatgtcc, atgtctggcccgtttt, ttgtagtaaatcctaa, ggcacagtggcgcgct,
gtgtttactgtcctaa, gacccacatgacggga, gatgtcctgtatcacc, agggcataaaggcatg,
gctcaaaaaaatcccc, cgctattttttaatca, acctcagegacacgag, atcttctccggcccag,
acatggttaaaccgtg, attagccgtaaatcac, tcaagaatcccttgag, ttaatcaagaagctat,
ttagctataatgaagt, tcctgaatgtggcggt, atccctggggatatgc, gtttttagccagcttg,
tccattacgttttttg, gcttggcccggagagg, tgcacatctgcatcac, ctcgttaaaaaattac,
cattttggggggggac, aactcaattcctctcc, attacctttagattag, ctgcagtcgttctttc,
cttaatgtgtgtattt, caatttcctacagggt, ctttcattagtccggt, gcgcggggactcggcg,
taatggtatctcgaat, cccaaatacgtgaccg, tttaccgcgttagcta, ctattcagccatgtgg,
tacaaaaaaaagggcg, gctgtgctgatatcag, gatgctaagaacgaaa, tgcctgttatactgag,
gtcacaggttttagtg, attccccagtcaggag, ttcacctcagcgcttc, agattaggtcatcccc,
tccatgcagaattcag, gtggctcacgccaaac, ctgggaagttctaggc, tagtgagactcctcag,
cgggggttttttttag, accactcccaacgtca, gcttctccgacggccc, ggcgatcaaaataagc,
gctggagttgcgatgg, ccatgagtaaagtgaa, ctcccatcagcatgaa, ctggggggctattatc,
tgcggtgtcattcccc, cctggtaatgcgatta, tataaatagcgaacat, aacgtcacgctttttt,
catttacgccttcctt, agctgtagggcactcc, tttcggggatggagtt, gaccaggttcctatac,
aaacctatttcgattg, aaacctattgtggcgt, gtgtaatgccataact, ggcccccgaactattt,
actacatctcatgtga, aacacattgctgcttc, agtgcttgcgcagggg, gaattctctatatgag,
ctttttccggctattt, aaagcttagtactggt, atttgtgagtgtaatt, ggacctgcaagtcatc,
cctctttgctaggtgt, tcaaccagaaggatat, atctggggggggcact, ctcaactgaaggggct,
ggtcgtatatatatct, aatcgacttgaaacat, aaacatagagccagat, tagctagtttatggga,
ggattgtttgtgttat, cacctccgcgtcccaa, ctctatgggaaatttg, atggagtcacgtaaat,
gaagtttggcgtgcac, ggtgctaagtcctcac, agcaataagggcaggg, atgattaaacaccagt,
cacgataaaaaaaaca, tccgtttggcttttct, actcaggtcgatcaca, tgcctaagcatcccga,
tctccaggatgagctt, actggagcacattgtt, agataaaatgtcggaa, ctgtctaacatggtta,
cacccacttttcagcg, aaggaaacttcaccaa, gctacattgtccagct, cactctccaacatccg,
agtgtgtgaggggggg, tcctttttcccccccc, tctgggtagagccgtg, cccgttactgaggtgc,
gcagttgggatgcctg, acttaaagtgaaggcc, taatctcaatatcgca, agcttatgctttgcca,
ggggcagttgtgctct, cggtttcggctgaagt, gagggcagttatgcaa, gccaaccttgttgtta,
cggtgtgattttttcc, ccaccagcacattgta, atttaaagcagatcga, ccggtgcgcgggactt,
ctgagcgccgatctgg, ggtggtcgcctctaat, gatacaggctgtagga, agtattgggggggtga,
gctgggcaaccattca, ccaggcatctaggtgg, ttacagtccttttgat, aagacatcgtctgtac,
gggcttgttgtcttta, cagtttttaccggtaa, ttctgtatcagctttc, gctccctgtatgcgca,
agttagaacctataac, ccacagactaaactat, gaagatgctcgtgata, cggtgttttctgccgt,
agggggtgaaaggtag, aaaatggggggggaca, tgccagttagatagaa, agttttacaatatcgt,
tccaatagggggaggc, acacccaatctgccat, gttcacttttttttcg, acagactctcacacta,
gctcaccaggagattg, atagtgattgccatat, cgtaaaaaaaatgtag, tatgttaaacgagggg,
tacaaaaaaaggcgta, ttttaatagttagagc, ttagaactcagcctgt, gtatctatcccgtaga,
gggagtccatgagtcc, gagaggggtaaggtag, gcttacctacttaaag, gcctacattgaaccac,
cgtggactctggtcat, gcagcttatgcatttc, ccctaatcttgctcga, gggagggttgtggtat,
aacttttgcactcgga, aggcgttttttttaag, aggataatgggcgtga, agctaatctggactca,
ggttacaagagacttt, gcatcccaccctagac, ggctttgtgggccccg, gccaatattgctttgc,
tgaggcagtgagctta, agatacaattaaggga, caccacttttattgtg, agacgcaaaactaaat,
ttcggcaggctaaatt, gcaactttggggtacc, ttaggcaagcggatcg, attgtcagtagctggg,
attgtctattatcacc, gtaagagcaacctttt, cagaactgggggggaa, ttgagctgcctccagg,
agggtggccaaccagg, tcgtcctcgagattcc, tcggggggaggtttga, gccgccgccagcgctc,
aatcacacccagcatt, tcgtgggaaggcctgg, gttgcaacaggtgttt, tggggttgaaaccagc,
tggcgtgcactattat, actccagattcgccac, tggattcagccagcgt, taggggtattgcttcg,
agttctgaggctcgca, ctggggaagctcctac, cgacccctccaaagcc, gatattccagtcaacc,
cgtacagaaagtttga, gacctagctgttagtc, tgactttcagcgccat, tcactagctactggaa,
tgttatccacagatac, tttttagggggggaga, attgagggtaatacta, ccaagcttactgttta,
ttaggggggggggtca, atccaaggtagagcat, gggtagagccccaact, aagccctcacctgagt,
ggcttacactccatgc, gccccacttggatcca, tcattccggggctggt, ctcaggttcataactg,
atctatttagcctgtg, ttgccgcagcactggc, agttccaaccctgaaa, tagagtcctcttatga,
gcctgcgtgtacctcc, ttagggggattaggaa, agctgatgagttgccc, gtacgctaaaacttag,
cacctcccggatgggt, tggaagtcagttgggg, acgaacagtatagcta, atatggggacgtcgaa,
tagccatcctggtctg, ccagtgaatcccctgt, gggcactctttcacac, aacccggggcactggt,
caagctaggcttaaaa, cagcgtcaatgccacc, ataagatttttcatgc, acttgccctggattgt,
tctgtcttcgcggatg, gttactgataatatga, tttggggggggtggaa, accttgggcctgtcta,
attcccttgtatctat, ttaggatgtactgaaa, gtagagctgtgtgcaa, aatctattcgtgcaca,
atgtccggcgcgggct, tctgatacaagttggc, gctataactccgcttt, tgtgcgtgaggagaac,
acctctaataatacag, aaaaaaaccatcctat, gctctctattaatgaa, ttagagcggttcctgg,
agaaacttggcaagcg, cttgaaatgaagccaa, tatagcaataaaaccc, ctctattgtcacaggt,
ggactcctggctgcgc, cgggcctgacttatgt, acagggttagacccca, caagggggggggtagc,
gtatagacatacgtct, ttactctgttgagatt, aatgagtgccttgggt, gcactttttttaggaa,
gtaatctgagatcctc, ctttcacaccttaaca, tggtgaaaagagcaac, tatcttacgggctgaa,
gactggtgtgtgggac, cccgcaggaatgtaaa, gcataatcagatgaga, ccagaccacttattgc,
aacacttatagattac, ggttgggccggggggg, caacaacttgttacta, ccttttatgtgggaag,
ttaaggttccaggaac, gtgcccggtctactat, acggggtttgaccgcg, ggacatatagcccatt,
gtgagttacaccctca, cccacattgtgctatt, aaatgattaaacagcg, agcaagttcatagtct,
taggaatagtctgacc, gccgtaaatcacttga, ttgtctgttttcggtg, ttaagtcagcttatta,
ggggcttgcttaggct, aaaacctcgaacagcc, ccctttttttgagtag, gccaggttaagtgggt,
cacgacacgtgcacat, attgtgtcccccccgc, cctctgcgaaaaaaaa, ccaaattgttgcaagc,
ggactccttttagtca, gtctccatacccgggg, ttattgaggcagctgt, cgtaacaacagttata,
tgggtgacagagcgtt, gccatgcttcgcttcg, ggtaaacatgtaattg, catagcagtactaatg,
acctgagttgacacgg, gctgtgttctacccag, ggaattttgaggtccc, cccgagcactttgcgt,
actttgacgagcttag, acctaagactggcaac, taaaagacttagcagc, acggagttagactccg,
gggagaactgcacatt, tttgaccaggacagac, gggacagttgcaggac, tcacgcaaaaaaagta,
ttttttcgttacatat, tgttagcgaattaata, ctgaaacaggaattgg, gcatctaaaaatggac,
agccaccaaggttacc, agcataaaatcgcagc, ctataaactcttgcct, gggcgtctacacagcg,
taaaaagccataatcg, tgcttaatgacgtctt, ggggggtaggaaccag, aacttaaaaaaaacgt,
taatacgtgaatatgt, tctggtatcttggatt, cataggaaaatttctc, tacctaactgagctta,
acccccgcctttatta, actcgtgttatttgca, tagctgccgacccctc, aactctttgggctcgg,
gctttggttggaggtg, ttgttctaacactgct, tacgcataagactatt, ttgtcttggaccagaa,
ccgtccacaatttact, tcccattccgcagaca, ttggctattggccagt, aaggacattagcctaa,
cccccacctgaatact, tactctgagcttctgc, atttagtgtttgcaac, gacatggggggcacgg,
cacgtctactctagtc, tacctgaattgtgcca, caagaagctgtagcat, atagacgccccccccc,
tcctttgtgcgccggt, ggctaaaatcggaacc, ccagcgctcgcgccag, ggtgtctgatttatcc,
ctgttttagtcttact, cggagtaaactacagc, atgggtgcaccccaat, gaccccccccatactt,
acggggtctcggcctg, agttgtactgaggtcc, gaatgtatgagcaagc, tagtattcttgttcct,
taacacttccccccca, agctacgccggcggct, gtggatgtaccagcgt, aggcactggagccttt,
ggcttgtctaggcaag, gagtcatatcctctac, cgacttgatcgtattg, gcacatctgcaagtat,
tatgtgggggggggag, acccatcctgtttcga, cctggagctgctacat, gtatgttactatcctt,
agacggggggggttac, atttgggcatcctttt, cgccccacatgtcgct, gtctgcctcattgtca,
ttttagaacagcatag, gaggctcccgtgggtg, gaatagtctgacctga, tctactaaggtgtcct,
atgcgccatgtaaggc, gacctgtcccctgtct, ctgcgagtccatcggg, ctccaaggggggggtc,
cagtggtctacacttg, ttccaggatgcgcagt, ttgctgaaaatcgaga, tcaaggttccccccct,
aggggttaaaaactgc, gcgtgtaacagtaccc, aaggtgcgtctgcccc, cttctctatcagcgtc,
ttagatggcccctcct, gggatacgtgggaagt, ccctggggggattatt, ctcatgtataactgac,
ctcgcacccattccac, aataaagcggactgta, agctcgtctcctcatc, gtcgctcaaataagat,
cacgcttttttttaac, ccacagaggtcatggc, gatgctgaccccctaa, gattaggggttggtgg,
ccactccaatcccgtt, tatattgaaccttatc, gatctaaaaaaaggct, catctcgaagctctca,
agtatgtttttttggt, gctcctatcctctcct, ttcaccaggctggcta, tgtggagtttaggatg,
ggccaatcaactcatt, gtcaccaacatactga, aagtatatcacttgct, ctgacgaagagaagga,
tccctgtatgcgcatc, tttatccactcaagct, agtaagtcctatcaca, tgaatacattttgacc,
cctacagctgcgtaag, gggctacaaatattta, tgcagcctagctgcgg, atcccactacagatag,
cagtatatcaggtcac, gcccgttactgaggtg, atgtatttgcttagtc, ttagggggggttaatt,
ttgcagtgcttttgta, gaggcccggtgggcga, atcaaaaaaagcgaat, ggggggggtaagctgt,
gaggaatgcccttagt, ctgatgaatcaacgac, aggtaattgttactag, gataatccagaatatc,
gtgcccggaccccgag, cacggcttctagcagc, tgtaagtattagtacg, ttgtatgagcccataa,
ttgcttcgggattcaa, cggcgggcggggggga, cacgtgatccccaagt, aaggggttccgatatt,
gtaacagtaggaattg, atgggggggggcgagg, ccgtaaatattgtatg, agtggaagcactaaaa,
agttagaccccccatc, gggtcaaaactgcctg, aagccgcatcccgctc, gataaccagctttaag,
gtcggttttttttagt, tcaactttataagtgt, catgaattactgaagt, acacgccccccatgga,
ttaggggggggtgagg, ctatgactggaaatac, ttctctgcaagataca, cccatttctatattgt,
ctctgtgccccccgaa, atctttcaatagtgat, gcatcccatgaagatg, aaaattaatcgaccat,
tagcccaccccctgct, attcattctgtatgac, ccatgagctttgagcc, cttagcaatcttggca,
cacgttttcttaaata, tgagggaaaccgtctt, tgtgttagtcagaggc, catctggaaccgaaaa,
agggatttcctatggg, ctataatggatatgga, ccatttaggaagttcc, tccttttatcgtgacg,
aacctcgaggttttaa, tgtggaacttttaacc, aggcggcgcggccttg, tccgcaacaaaaaaaa,
tattttagctactggc, cccacagtgctgtcga, tccagtcgctttttcc, tcttgattcccccccc,
tagggttcaccttgga, agatgagacgattgat, acatgaaccacctctc, actgtactgtcaatca,
acatgcagcttagggt, tcccccctcagctgat, ttaaggattggcctgt, gacaattaatttcctg,
acctaaccacagcact, ggatatgaatgggtac, ggcttaggcagcagat, ctgatgaaatgtattc,
cctgaaataacgccgc, aggaaaagtgcattag, tagtctcaacattgac, acaatgtgcgctagcc,
ggaggtggtgcatagt, tcccagcttacttcgg, tcttaatatatagtcc, tccaatgacttgcgag,
tgggcgctcttagtcc, ccctagtaacaaatgt, gccatacagtgggcac, ggggaggctacgggga,
aagaagtatgaatggt, atctgacagcgtgtaa, accctcgcttctgcct, gttctcactgtttaga,
acctctataagacagt, ataaagtgctggtgac, attttggcccctaagg, ctatacaaattgtctc,
tgaaccagagtatctt, atgcagactagagcta, gattaaaaaatcaccc, gcccttagacttctcc,
ttcgtatacatatagg, ttatgccccgcccctg, taagcctctacataaa, aggcaaccatccggtg,
gagttagacgtcccag, ctgtatgtctcttgcg, aggataaaatgcctcg, tgggatcgaagccgga,
gactatactgagaatt, gactggcatcggatca, gtcatatgctaatcgt, gtaaaaaaagagagtt,
tagaaatggggggtag, cggcttctagcagctt, cgtccttaaagtttga, accaatcagaggtata,
ggaattaagaatgtcg, caatacagtgcatatc, aggatcactgacttat, tttaatggttaccgtg,
ataatgatggccccgt, acatcctaggtgaagc, ctgacccacattgcct, aacaacgagatgattt,
caagtcaaagcctcat, gcctgcctcctctgta, cttgagagtgcataaa, agcaagcaagtaggtc,
gaccctgcaaagaatt, gcaggcatgccggggg, acggcaggcagattat, ctgcccccccgaaaaa,
ccatccaggatagatt, atagggttaaatggtg, ggaaaaaaaacgttat, catgcatggcgggctg,
ttacatgctgtcctcc, aacgcttttttttact, acgaaaaaaaaaagtc, tttaacattgggtgtg,
ccacaaataacgccgc, ccctgatgcccgtcag, ctggtcttggccttgt, tactagtgtttcatct,
aggcactagaaaccac, ttagacccagaattat, ttggggggggcaggta, ataaagcggactgtac,
ggattagtaaactgtt, aaagtgtcaaaaaacg, gtacctccattctgga, cgtgtttttttattta,
cattgcattgcaccac, ccccccatggacctgg, agggcataggcccagt, gcccatagcctgacag,
agcctatgaggtaaag, tcatcaggatctaaag, ccccggggtgcatcgt, ctcactgggttcccca,
gtttatccagtcacac, tattgttcaaagtgta, cagatttcgaaacctg, gtccacagtcattgcg,
tacgtcttctccattt, gcgctctggaacactc, agtgctatctaatctc, aagtttttggggggga,
gttacacttacaaaga, gaccccccccatgttt, gcttcaatgttaggat, tctattgtagttgtct,
tggcagtgagcaccgt, acaggcaacgtaggat, caagaaatggcagtac, tgtgcagttggtattg,
gcgaaattccgtctac, gcacagcaatacacgg, tcacaaaccgtcaaag, gtgcatcgtgctgatt,
gtcagctgatttattg, tgttagacccttgttt, gaaagtcataggggac, ttgccagaatttgagt,
gccaataaggttaaac, atcgccttttttaaat, cgggctgatcacgagc, agagaaatccattacc,
cctctaaaatgactgc, ggttcctgatgcacta, agatattaagtcaatt, cccagtagcccattcc,
agaatgagggggatgc, ggttatcctaagaaat, gggtccagcgcctact, gttttcggctgggctc,
aagtcccagccccccc, taggggctttttttat, ggggctaggcagttca, gataagcctagtttaa,
cttatggaatgatgca, gacacccaccgatgtg, tctcccccgaaatttc, acggtgtataaatctt,
acactcctgccttagt, ttgtattgtgaccctg, cattgtcaccccccaa, tatcacaaaaagaacg,
gaggccttttttgggc, ctatgctccatcccgg, acaaagatcatttccg, attccggacgggcatg,
tccctgtcttatggcc, catgttgaattcttct, ggaaggaccacatacc, ctaggcaataatggga,
gttttcaccggaaact, gttgcaaagtattgcc, tttttactagcgactc, gtcttaacagtgtgtg,
ccgattttttttacat, cacaagacgggccagc, gtgctaaactcatagg, atcgggaagagtgggc,
gcaggtgtcgcttaaa, cttcattggttagttt, tatacaaatcgtcaca, ggaggtcaaggcataa,
agccctcggttggttt, gtagcgtgaagcctgc, tgtggcagagagcgga, tactgatctataccta,
gtttgttatggtgacg, gagtgtaaggaatttt, cgtaggttacagagag, gaagaaattaggcctt,
actcgtgtttttaaat, gctggtgctctttgat, cccggtactttgcggg, gctctgtgttataccg,
taatgtaccccagctg, cgggcccacacttctc, agcccctgtcgacacc, tgaagggtagcctgga,
gtattacccatatgcc, cccagtctaactggcg, taaatggagaactctc, caaaataagcgggggt,
tcagaatcttatagac, aattttggctggggcg, ctttgcactccttaag, gcgattgcttttggtc,
aaagtcacagcgcttt, tctacttccgcagaat, gtaattctactgcatc, ctacagccttacccta,
aggatagtgccacaat, cagtgcatgacatctg, aacccacaattggtat, ctcgggttgtatagta,
aacttaaagaaagggt, agcttactgagagcgg, ttaagcagctgatgtg, tgaaatatgctgccta,
aggaatgggagtattc, agcagtgtagttttat, ccacaggtttatatct, tcacggtagtccagta,
caaattccgtactgat, gagtgtctacaccgca, tttcagatacctagct, gacaacatttttttat,
gcgggaccgggcgcta, aaggtatacaacctcc, aagaatggtcttgcta, agcacaagaggcggag,
tagggacagtatcttg, atccccccccatggtg, gtcagatcagaccttt, aacattgaatcactaa,
tgcttggctgcacaga, cgacaggagttaaact, ctgtactcccccaatc, ttgccccccccgccaa,
ctatttctcgctgggc, atgatgcaaggagagc, gcaacacaaagcgagc, cctctgttctttacac,
gatagtcaagacgcag, gatggttaatagacct, acatttccccatctta, tattgaagcctagcta,
gtctagatcaattatg, tttaactccgtctccc, aaaacaggcgttattc, aggatttaagactaca,
aaagtccttatccata, acggtatgtactctta, cagagttgtactccct, tcctcccctagcagtt,
gagagtgcactaggtg, cagtttcagttcgccc, ctatacttctccagca, ccaaatgtattcctac,
gacgagtaggttatga, ggtttaacgtgttagc, cttggggctgcggggc, cctctgttttcgcagc,
ggcacgtgttatgtaa, atacctccttcctaag, ccgcaaggtgctctct, ccattagcatggaacg,
cggcgcttttcagttt, ggtatcaagccaggta, ctcgaatggtaactcc, cactctagaatcttat,
gcagtatgctgggtga, tcctaggctccctatg, gataccctccccccca, ccttgaccacgaactg,
caatttcacctcccca, caccagtgccggaaca, tgctggcatccgtgtc, agcatctcctgatcac,
tatccactcaagcttg, gctatcagttttccta, acaagtctttgtctcg, cactgcaaatgttggg,
cctggtaaatcaagat, ccaattgaggtttcta, aggccgcggggtactc, tactgctatactcaat,
ctcttcacaaggtctc, ttgatggggttggctt, tggatttccagactac, atcgttgaaacagcgt,
agtgagtgagttcgat, ttcttccacctggtgt, actagggaatatacag, cctctatgtaaatact,
gtcccttcatcttcgt, ccgtagggtcatttga, cgggaaagatgggggg, ggcgtcttctgtcact,
caacgaaaaaaaattc, tagatagctggagggg, ttctcggaagaaaccc, ggttgtcaaagttccc,
tattggaactaccacc, cggtgttttttttaag, taaaacagtatggcga, cacaggcaacgtagga,
attcaggacatatgcg, ggggatgaccgtagtt, acaggtggggggatgg, catcagtggaccttag,
gagcaaaaatctgatc, gcaggtccacttctta, attgcaagcaggacat, ccgtttggacagaaat,
tggacgagtgacctgt, tgatgctattcaagac, aggatcagcacggggc, ccgtgctctcttaaac,
catgaaccaccaagta, ccaccttcttggcttc, agcaatacggtagtta, tcgcatcaaaaaaaag,
aagtagacttagtata, agccagtgtcgccgct, actgaagatgctcgtg, catcaggaaggaagcc,
ttgtgttgcaactagg, ccgcaccagtcagacc, gactatggccccactt, atatgtagtagctcaa,
acacgaaggtggagga, ccggaaaaaaaaggca, ggcccttagcagcaac, atcaacaagttttacc,
tggccatattggattc, gtttctctttagatcg, atagtagactgtgcgg, atgtattcctttggga,
cttacttccactgctc, aactgcagtttgctat, ataacagcctgcttgc, tttgaaatgacgaata,
taagatcaagaaacgc, acaatttatgttttcg, acttgcacgtcttatt, gaggtccaaacaggca,
tgagggctgtatgcag, tccgccgtagatgccg, tgagtaagcttaccca, ttacatagaccaacag,
ttgcacctctgagacc, gggggggataataact, tgccaccgaggtgggc, tgatctaaacgcgctg,
aggccagaatccttta, ccctcttgtaatgagc, caggtacaggactgtg, tgtctcccaagtccat,
gggactattatggggt, tattgaacccccccac, ggccccaaccttatat, tagagcaaattctata,
tttgcgaggtggcagg, ctagactatttgtatc, gccgggcgctatggcg, acctagaggttaggaa,
ctggcacagttaagct, tgtgataatgccctta, tcatacctcactctca, tgcacattctgcatag,
gacaggtttagaaaca, gctgtgaagggatggc, tactctgtgggggcat, cacttttgctgagagc,
aaagtaatacgtaaag, gcaatcaccgtctttt, gagatgtcccagtctt, attcaccggatgggct,
acaggctgatgctgtg, gctatgacttaagaga, ccttagatgccacttg, tatctaccccccccac,
tcatccggctgcacat, ttgccttattatgcaa, cctggtggccgctcag, tcaatgaatccatgtg,
gccttcctcttgtcaa, cctgggaacactagac, gagcgtctgcggaagg, tgcacatgttacctag,
ggggctgtcccgcggg, cggctaggccgccgca, ttatatcataacaggg, gaggtaatgcttgttt,
acttaataggctgatt, gttgaagggagccttt, actgaagctaagtagc, caaatccaacctccaa,
tcgttaactctttttg, gggcttttagtctata, gacgcaattaaaatac, tttagcctggcatttt,
aacacagacccactgc, ggcttaggcgggtgta, tttttttaaccgtatt, aaacattggagtcatg,
gtacctcactgggttc, tgatattaacaaccta, aggatgcgcagtgtaa, aaaacggggattgtca,
aatgacagagtgcatc, atgcgtcatgttcagg, tctgacgtttttgcag, gatacgctctaagaat,
ccaaaagtcgaagttc, gcacttctgtataaca, gcctgcgcaggactat, tagtgcctaaataaac,
cgcgcacggtgctctg, ctcctggcaccatact, gatttaaccacgtcct, gttcgggttatgagtg,
tccctaagtatacteg, atagggaccctggaat, ggatggcctgttcccc, ctcagcattccatgca,
gtacttgttatatgct, tacttcttggtaaacc, tagcttcactccgaca, tggtggtcgcatgaat,
aatagtttaaagcgtt, cctcattttttcacac, cgcctcccgctatgca, gttagggtggctcctg,
gcgtcccctccactcc, atcttaggagagctca, atccggagtgagactc, aggcttgtgctggctc,
cctgaatatgcttgtc, aaaaaaacggggacat, gtgggggcgagagcat, cctgccccccccgaaa,
gctctactccttcttt, caaaaaacggggctac, accctgccgaccttgg, gtggtgtcccgggacg,
gattccttatatgcca, taggtcacttcatgta, gtctcgatgtcctgaa, ctggcatgacttatgt,
cttagttgtgtattct, agggtgcagatatatg, gcatcatgccatgtaa, tcaacacaggacccac,
aagggtctgcgggtag, gctgcactatttgacg, gggcctcccctgccgt, acctgtcaataacgtg,
tgttgatcacaagcta, gttccaggaacaagca, cgcagaggaccattgt, gggaaaggcccggcgg,
ctatcctcgctgatgt, cagctgtatgaacata, atagcttgctccaacc, aaaccttaaacaggca,
ttaaagtctggaccca, catgatgcagcatacc, ctgtgtaatgtgtata, gccattgtacttctgt,
tcttcccggtctttgc, ctagaccttgagcatc, agcgtcaatgccaccg, cagaccgcagggccct,
acataactggcctaag, atgcccccccccagtc, gaaaatctggcaagta, cagggccgcgttgcgg,
tgcgggtggacaatta, tgcttgtggtgacgga, cgtcttacacattcat, ccaaccgcccccccca,
cctctttgcatgtgta, gtgagcctaaattctt, ccacatgtcgctggat, tttttcgtatgttgct,
ccaagtatgggatata, tttacatagggtgcag, tccttagagaagtgag, ttcggtatgtatattt,
gtactgatgcttgcga, gccacacaccacttaa, ttgcgtacagtaaaat, aggatatccccactat,
agtacaatggctcgac, aactgtatctagtatg, tagtgttttttgcacc, agttagggatccccaa,
aatctcaatatcgcag, atatcattcgattact, tgacctagataattgc, cccttatgtttgtcct,
gtacccagtgtgaggt, gtcccataggggagag, ggcccacaggttcatt, cctttggccttgtccg,
ctgatagcatcctctc, ctctgcttaacgcatg, ttatctacaaaagcta, catataaaacccctta,
ccctgattacaggcgt, cctctaacctgttagc, aggatggttgtgctac, tcagcggggcgattcc,
ccatgttttgccaccg, gcgcggccttgcgacc, tgtgacccaactgtaa, cgataaaaaaaacatg,
agggtgggccgtgaag, atacgagaatggcata, ctcgagggtcctcggc, cttttaccctcgcttc,
acgagttagactgtgt, atgcatggttggaaat, tgaggatgggcctctg, atttacccaactcggc,
caaatacaagcggatt, cttaacgcatgcaaag, gtcttttacactaaac, aagaagaataccactc,
tagctgaacagtcact, tcggcaatgcctcgat, aactgttaccactgtg, gttagccacaattgtt,
catccgttgcatggaa, tccctatgtgaacaag, tttttctcgtgacagt, tcaactggtgcgttat,
caccatagtgggggag, aaaccgtgcagtgtac, tggctttaaggttccc, attttatcgaaagaca,
acacaccagtgtacag, ccccataatagcgttt, gtgatgcaatgatagc, ttagagacagagttag,
gatgcaggtctcttct, cgtggttaaacctcat, atgctccatcccgggc, tcttgccccaatgcat,
accatttttagtgcaa, ttcgggcttgggctca, cactctgcactccctt, cattcaagaggacgca,
gtagggggggacgggt, aagtttggcgtgcact, ggctcggtggtttacg, actgaccaaaaaaggc,
ccttgtggatggatcc, cagctaggggacccaa, gaaatggctgggcttg, ttttttacaccatatc,
tgggacgatggctctg, gtgtctagacattgag, tattggggccccccag, ccgactcccctgcagt,
gtgtcccccccatcat, tagtatgaacacaagt, aggagccccgctgtct, cttgtcatcaggcgaa,
gacaagggccgcgagc, ctagggggtgaaaggt, attcgatatatgtaga, tattatgcactcttgt,
gggccggactctgcca, cccctaaggggccaag, tcaaggttattacatg, tggtgccaggtctttg,
cggaatctcgctgctt, ggccacaggcccgatt, tcggaggcacaagaaa, tcgttgaatttacctg,
tgattagctcatcata, ctgacactaggtctcg, atccccgtgtatcccg, ttaatgtgattgggca,
cctcttactgggattt, gctattggccgacaga, ctagtttaaggattgt, gcttcattggggtgat,
gtagcagtgaacgaag, gtgcaccacttggaga, tgcgctattttttttc, ctaaaatccaaccgca,
ctcagaggcagtctac, tcggggttttgatatt, agcgggactacatgca, gggaataagcttccag,
cggtcttgtcggtgga, gttggtcatagccagc, ggactgtggacgggat, ccggtccatttctttt,
acggcttcagtggtgc, aaataaagtctcacgt, agctaatctactatca, tgttgacattctccca,
gggcttggggggggga, attcacttaaaccggt, ataacttgatgttggg, aattttttagaactgg,
gagagttaccgtaagc, cctaggtaatgttagt, ttgttgtccatttccg, ccagtgtcgccgctca,
ccagctgtacgaaaaa, cgtgggcgtgtgcgtg, ctttagtatgttcaac, tgtgcaagctggggat,
aaataagaacgttatg, acgcacaacttataac, tegaaccccccccctt, cctcaaaggacaaaca,
aataggtgaatacacc, cgatgaacgaaaatga, cttcgcaattatctga, gatagagtgacttctc,
aggtcttttcagcact, gaagagttccggttca, agttgaaaaagcttgt, tttgtagacatttgag,
ggtgggcagtataggg, cttgttaagtattcac, tcagtggcattgactc, ttaggagcaccgagac,
accccttggaacaaac, cttgctaaaaaaagcc, ctaaccccccggtgtt, agaggggtagtttgag,
ttcgggttatgagtgc, tagcaactggttaaaa, aatgtagatctttgct, gccttcaaggttccag,
cacgctgggaaccact, tatacccactatcttt, tgactctaagctgcac, gaagtgctgcgttgcg,
tgaaaacatccagccg, ggtgggggggacggta, cagggcaaggctcgga, cgtggaccctctcggt,
agttattgtccactcc, aaatgtaactcgtgta, agcgtttgtttaacca, gtagtgcttaccacta,
atttccgtgaaacgca, tactcataatctatgt, ttcctgttgcagatga, acagcctttactgggt,
ttcccccccacattga, cttacgggctgaaggc, tgcgaggctggggctt, gtctacaccgcagtaa,
gttgcactttagtaac, tgaatagttacagtgt, ggccgaggccgtaggg, tagagtgatgagttgt,
ttcttgcgctaatctg, gatatgcacgcagaaa, ccatccatcgtacagt, ctatagagttgtctgt,
atactaaaaaaaggcg, tgtaccactaaatgac, tctcttccctaagttg, cgtaatagaacatctt,
taacctggatcttata, gttctgacgccactcc, actcggcaaacttagg, tgtcgcacatttcttt,
ggcctactgtctttgt, tactgcaaggattcta, tgttcctgggtgattc, aggtactgtgttaaca,
aaatcgtgttttttgc, cctgttatgaacctgc, tttaccaactgcaact, ccagggccagcgtggc,
tccattctactggtcc, ctttcattaaggaagt, tgatcaataacgtttc, ccttaggttgtcagcc,
atggccggagtaggga, ggaatactaccaaaag, acggttcctggctgtt, ctgcccggcccgtcgc,
aagggcttcgcatccc, gctaaggggggacaag, accagtgcaccggttt, tattcaccgagtgaga,
gcttgcaaaaaaaggg, ggcgggtcattttgca, tatagacgtatagaca, catgattcgactccga,
ctggaactgcagcccg, gctggtgtgccgagcc, gttgcttgtactcagg, ctaaggggggggatgt,
gtccatatttggttaa, gcttggcttattacca, gtctattgcttctaac, catcgcgtggtgacag,
tgggtctgtaaccttt, acttctctatcagcgt, taccttatgagcaaag, gtcctgtatgacataa,
atcccgagcatggaga, acgatactgttattat, tcaggtattgagcaat, caatggactgtcaaaa,
atccgcctgcatcagt, agcattttaccccgcc, atggccccccccccag, ctgctgatggggggga,
tcatatagttcatggc, ttaccttagcttcgtg, gggtttttaccctgct, gaggagtctgagtttt,
cgtgagcagtggttta, tgttccagccctcgat, ctttcgagagtctgac, accettaacccccaat,
agagctctgaccgatt, tgttgaacccttccct, cgctaggcggctgcgg, gtcttactgtgtccca,
tcagggcatcacctaa, tccaaagtgttggtcc, gctactggtgcacttt, tttcccaagagcttat,
aggttatgtcacgcat, gagtccaacgttttct, ggattggtgttgactt, catctgatagaacacg,
ttctgtaagaaggacg, gatagtggaagtgtag, aggttaagctagcctt, cggcttcagtggtgca,
acgtgtgcatcccacc, ggaccagtggtagagc, gtgggggggaccagtc, ttcccctaaattagag,
tgaagtccctaggaac, taggctctttttttcg, gcattcaattatggtt, gctacaagtcttaaat,
ggatacatgcacataa, gaagactaggggggtt, ccaccagtgccggaac, gtaagtattagtacgt,
cactaagccagcttct, atagccaacaaagttt, ctaaaaaaaacgtcct, tcctccaagggggggg,
aaatgaggacggggcc, aatgcactactataat, atttttgggaatgtcc, tgaggcctccgctcct,
aatctaaggataccag, ctatgatgtaagcctg, cgggtccagcgcctac, acgatgcagcggatcc,
atccacgtttacaatg, cagggtttgctgttaa, ggaatcttaggaccaa, cgcgcacggtgcgtac,
ctegaagctctcacgc, aacgtgtagaaaccct, cctcgttgagcacggg, tgggagggggggacac,
ataggagacgtgcttg, gatccccccccatggt, gtgatattatgatgga, ggtgggatttggttcg,
tagcgctagcgctata, acaaaataggttgctc, cttcatgtgtggccct, agtgctgctgagtact,
tctgagttaccttagc, ttgcaaagcacaagaa, aattatagtaacggtt, gacgtgcaggtgagga,
tgcaggcgggctttca, atcctcataagttttc, tccgcaggcatgccgg, tgttaaaaacgttact,
gcctggtctaccaatg, acatcagaggataatc, gagctgctacataagt, gtgaggagaaccgcct,
attggaggacatatgc, agcgccttggtcaaac, ctaagacaagctacag, gttggttgaccctgct,
ttactggtctgtgtca, tacaatgcggatacct, tggtgtgagtgtcaaa, ctaggctgccgctagc,
gtcattcccccctatt, aaaaacgtcatttaat, cgtagactacaattcc, tattctagcaggacca,
ttgctagtgtcattta, tgacgaagttttcagg, atgccagagaacttag, ttttaccattagccac,
ggcgatgctgagatgc, tttggcaaccaaccac, aaggcaatacatccat, tttacgtactatgtgt,
gtcctcctgttttgta, tggggtgcattaggtg, tggtgtgccgagcctc, gtgttatagctttttg,
cttcaaaaaaatggtc, tcatatctggatacca, aaaaacgtctagataa, tgttagactagtatta,
acgaaatttttaactt, gctccaccttgcccgg, cacgtctgcacaagac, atatgggggggttaga,
tttggtctttacgagt, atgccattgtggacgg, tcaaaggtcgataaat, ggactttagtgcaatc,
gccgggcgcattgcgc, aaccccgtgctctctt, tatgtggcaatgatga, gagcaaaaaaggaggt,
tagaccgggtggtggc, aacacaatctgaggtc, aatgcccctttgttct, aaagggaggagcggag,
ttggaacaccgcgcaa, gcttctcccccgaaag, aatggcaccccaggat, agctaacgcaacagag,
ctacaggcagcggatg, attaagccagtgtcgc, tgatgtgggattagta, gaggatttcaacatcg,
tacacattaggctgtg, aagaatattccctata, gtattgcttaatgacg, cctactggacggggcg,
tttcccccgatggatg, agaagcccatcctgag, gatagcaggttatgtc, gccctttctggttaga,
gcacacaccactcact, caccaacgattcacat, ggctgctttatattca, catatccttgtctgcc,
ggtgagcagtgttttc, gaacgttaaatacata, tgaagtgtgaggatcc, ttgcgagttttggtgc,
tttgttagggaaaact, ggggcagagcaaaaaa, agtcgccagcaagaca, ggtcccattctatgct,
tgggtgacacgagtta, gagttacttctcctgc, atcatttccggaatgg, cccactcaaccactgg,
ctggcttaaaaaaggg, tcctctatgaaggagt, ccactgtcgtcatctc, gtctgcatgcacccct,
tatctcattagtagcg, cgacgccggctaggcc, gctctaaaaaaatatg, agccacgcaggtttcc,
agcaccactcttagag, tagcctggccactgcc, ttgctctcccttagca, tgattctgggatgact,
aaaacggggggtaaat, ctgaaaaatggacacc, gaccctgcccttagac, cagcggctctccacct,
taactaagtgaatggt, aaggcttagtcaccat, tattttogtaaaaaca, cctgactgtcccaccc,
tgatagtgcagccacc, ttactatgaagggtct, ccgctgctttatgaac, gtgataacatctcact,
gactgatcatcaggaa, ttaatcgaccataaat, ccccatcacattaaac, cctggagttagaggac,
agtctggaccatttgg, gcgatgctgtttcacc, ctttgtaactaattcg, ccggtttgtactgatg,
gccatgtattactaac, tgacactctatcttgc, gcggcagggccgcgtt, gctgtggccatccctt,
ttaacagtccaggcac, gtcttatctgctgtaa, gacttcctgaattgta, gaggtgatcgccctca,
cgccccacacctgtta, tccatgatgccttaat, gttctctgacacagtg, agctgtcccccccttt,
ttgcccccgccgcctc, gcaatagagtcttcaa, caagagacctctattg, acataggacgttcctt,
tgccgctgctttatga, ggcgtgtgcgtgagga, agttagtgtcatgggg, agccttgcccatcggg,
ccagcaatattgctgt, tcccagcaacttagta, ctgatttagctttcca, ttgaaacgaatataat,
tggcggggggggcgtt, tgtgaataacaggcat, tcactgttgcatgatg, ccacgattacagaacc,
gatggctagagcaata, ttggggggtcctgaaa, catatctaacacggag, ctcagctctctctagt,
accatttttttttggg, gtttagaagtcacacc, accacatagcctctta, gggtgatgcaatatct,
tatcgccattttacat, acaccaacccccccag, actagttctatactgc, tttaccacggtcacct,
agttgggggggggagt, gcggagacagtatgtc, gtggtggcagtcgcca, tccaaattgtaacagt,
gttctgcctctactgc, tctcgatctgatctct, gtcccagagtccggcc, tggtaggactaggggg,
aaggacgcggtggctg, gccggatccctgagtc, attcctattcacccat, gctgtacattggtcct,
ggccgcgttttcaccg, gttgcatgattgtacc, catcatgtcagcgatt, aagtaggagcgaacag,
cctgaacttagatgct, tggtgttacttctcac, ggagtcccgaggcact, tctttatgtggtcctt,
ataaagctaggatggt, ttgagtaagatgctag, aagcctctaattgtgt, caaagagcacggggtt,
tttccccccgccaccg, tagtaccttgtaagag, agacgtgatcccaata, tcccccccgaaaaaat,
gattatggcgtctgct, ccaaatacgtgaccgt, aaggttacatctgtgg, tagaggaggctctata,
gctccagaccgtgcgg, gggcgttcagggactg, taggcctagtaatctc, aagtattcttccatca,
accatagtgggggagt, cccggttccagttcaa, gggtttgaccctgaat, actacccaatttacaa,
ccctcgccccggtggc, aacccacatgctgaat, ttcccccggggggagg, ataggcctagtaatct,
ctacttaatggtcagg, ctacttttttattgcc, cctttgcggggggctc, ggtctccgactgttgg,
ccagaggttccttgat, caagccgggcagctcg, gggctaatgactaaat, gggtaggaaacagtac,
cggtgtcattccccta, agtggacgctaccggc, tcttcaatctattcgt, tgtgtcacaatcacag,
ctctccctctcgtgcg, tgaccagataaagttg, catggacctggtaccg, acgtgccggtttgtac,
tcaatgtcaggtgcgg, caactagaacacttgg, cgacacagtgatattc, ccaagcctcatatgtt,
gcccccgaactatttt, ttattttatccggcca, gcattgaagcaagctg, aagcctgctttggtgg,
ttttgcccgtttttgg, ttccctacggtatcac, attctaaaaaacacgt, gagaggccttaaatta,
taaaatcgcagcctca, atccaaaagtagccag, tggttcagcgcattca, actccgtgtaatttca,
cattcaccgcactgga, ctcgatttggctgggc, ggagagcatgccggac, taagttacgacttttt,
ttggtctttacgagta, gactacgtgccttggg, ccttttatcgtgacgt, ccctcaaggctgaaaa,
ggaatccccaaggtcc, tcaccacgaaggtatg, acgagattcacattgt, cgtgcacttttttttg,
atagacctaattacct, ggtgggcgttctggtg, actaggaactgatgga, gtagtagtgttttttg,
gcacttgatgttatat, tcaaaaaaaatccgaa, tggccaggagaaatgc, gcattcacacgaaagt,
ttccgcgctccgcgcc, atggggccagggggac, atgctatgttagaccc, tttcacagcccgtgtg,
atccctgtagtttggt, cccagatgatatcagg, gacaatgatagcataa, ctatgggcgcccggct,
ccgcctgcatcagtct, tattccttctaatgcc, ctttagacttgcaagc, tccaatactataaccg,
tgcctaatatggattc, tgaagcacaaattgtc, gatctgttgggtctaa, agtgcatactcagatt,
aggtcgcaggagtagc, ccgtggcctggtggtc, ggccaaactcaaaaaa, tagagttatcacctga,
atatgcactacacttt, tggtggtgggcgctct, aactaataggcttcac, tttgggatgaagaggc,
ctcgcctggcctctca, gcgggcacattgtaat, tatcgattttcttatg, ggaagctctaacccat,
ccggagaagaatgtgt, ttggataagtgcctgt, gatgcacaccatttgt, caagaacaacggcctc,
ccatacaagagttatg, gtaagggcagttacag, ttgagtaatacataca, gggacaaaagagtctc,
aatttgtttgttcccc, tgctcccagcccttac, ataaaaaaactatacg, taggaattaagagctc,
ttctaacctaagaggt, tcaggatatccttacc, aaagtggcaactggca, aagaccggaatataaa,
ttaggaattgtggtga, atcccttggagttagg, ttggatatgggggggt, tttgtgccccttttga,
acccaacggactgtaa, ctttaacgtttagtga, aaggtcctagctctca, ctgcctgagcctcgtg,
gaaccgctccagtctg, gctaagcggtgaggat, cttaagatccccatgc, gatctatgtacaaaag,
acaaatgctgttccta, gatgggacctagaaac, ctccgcgcggggctca, caacatagtaaactgg,
gtcttaatagccatgc, ctcccctgctagttgg, tggtatgataattaag, catcgctaactaggcc,
ccacgcttgcttgatt, cacagctggggggcta, aaatgtaccgtaaatg, gggtaacccagtctaa,
atagtataccctacat, tgtaatcttcactact, gtcacacatgatagca, gcaaaagtacagccag,
agagaggctcttcccc, ccatggaggtcttagc, ttggggggggaagata, ccttgttatgtcatat,
ttttactcacaatcta, ccaactgcacagttaa, aagatatcctacctta, aaatggaaatcccgca,
gcatactgagtgttct, gcaacgtagttagacc, catgcaaatccaagtt, ccgctatgcagctcac,
tccttgtacagtgact, gagaagacaatttgcc, atgggggggtcagcaa, agccgtgccttccggg,
tccttagttcatgtat, tgctgcgtttggtttc, caggtaatcagcccat, gagactcattaggctc,
tttacagggtctcctt, cggtgtatttgtgctg, gcttaagtcatgactg, cctctgcggaaggacc,
agcccccccctcccgc, cctgggcgggcacatt, gatcacatctaagtaa, caaaggtggaagatac,
cgtgcccgtgcacacc, gtttgatattagcttc, tctaacctgttagctc, tctccctctcgtgcgg,
attcctcttttgctgg, cttagcacttagacta, tgcgggtcctgattgt, tccttccattgcccgg,
gtaagtctattgaaag, aggtcagggatcgtct, atcagtatcataagtg, tgaagcatagactagc,
atatcaatcttgataa, caacctatgtgattgt, aggacctcttcagacc, gtcagttatttacctc,
gattataagatttgct, cacgaatttttgattt, caatgtgtccataata, gagttgacgtgtggta,
gtaggtcacaagaatc, taggatacataaagcc, ggggatactttacaga, tccattgtcctactga,
cttgtgggtatctatc, ctttaattgtctcggc, gaggctacccttatgg, ctgcacttcaacctac,
ctggcatgcatgttct, cttagtgtttaaccac, gattacatgaaagatc, agtttaaggcagacca,
atgtgttaggaatccc, ggcacggaaatgatga, aaactctgccatccat, gtgttttgacaggcaa,
tcagcgtatcccatgc, ggatttagtaaaactc, cttggcctttgatagt, ttaagccagtgtcgcc,
ccgtgttgttccctga, cacagtttgcgatatt, gcaacaaacgctgtta, gactctagagagtcag,
ataaggagtcagcaac, aataaaccagatcgct, cagtaagcttaatgtt, ccctcgatttggctgg,
acgcaaatcagcaaac, ctggtttaggaggaac, ttactggaattgataa, ctgataattgggaatt,
cgtgggtgccatgaat, aaccccccccctcctg, cgcggccgtctcaggc, acgtaaaaaaaagcca,
ctggctgatcactcac, tctcacatcatagttg, ataccctgtttttcta, tggtaaaaaaaccaat,
aggtgtctaattaaca, ttaatccatacaaagc, cacccgctctagaagg, attgtggtagggacca,
catgcaaaaaaaggac, ccctttaatgcagatc, atggggacctatgtgt, gagcagtatcaactgc,
gtcagatttaaacaac, gtcggggaaaatagca, acccttagctgaggac, acccgggggggcagaa,
ctcttaattgcgatgt, atgttagacccttgtt, tgagatagaaatctac, gctgacgtccgtaatc,
aatcagccaactaatc, ctggtattacaagtac, tctcaatcagagagat, ccttctattagacagc,
aaagtatttatgagtc, gagagttgcaaaaaga, tcccctaaggggccaa, agttcgtaagatctgg,
ggtgtcccttaccaag, tccatacccggggcca, ttagctcaggatcttc, ttcacgctggaaccac,
gcatctcaatagacca, aagcagtaccaatatt, cttagggtttgttatt, ggtgccactccttatc,
tcccgaattcagtgtc, acgaactgaatagaca, ggaaccttgtggccat, tatatcccggggggag,
accgtttagatgttta, aggtccacttcttatt, cccccatggacctggt, gttgtaagcccttaca,
gccgaaaaggaaacta, ctactccccagtactc, tttggaacaccgcgca, gctaatgcgtacctca,
ctttctaaccccccgg, caaatttgctctaaac, atcctgtacctacatg, catccaacttggtctc,
atcctcgctcacttta, actccaactggagtag, atggttaaacccctgt, agtcagtgtaagttga,
gccaccgctcttcttt, cctcggtgcctgtggc, caccggaaaattaaat, taaggataacgttcca,
atagaggccttcaggc, gtgtgaggtgttcaag, ctttttgtgccggctt, acgccttcctttgtct,
tgaaaaccatagcaga, accagaaggaacagac, gacaatcccttaaggt, ttgcaggcttagtgat,
tggcccgcaagcgctc, atctttgtgccccgac, agtcatggtgcacatt, gcattggggaagcgta,
ggcctttagtagcttt, cattgagagcatttgt, ttaaagcgttagaggt, gcagggactataagcg,
ttgcttggtgagtctg, acatcctcacatttag, ttttaacgtgccggtt, ttcccctatgaaacta,
atgtttcgtattagaa, ttataggtgctctccc, cgtgcactattatgga, aagttgtcccccccca,
gtaacattgcacctta, gggctgagtaatgtca, cgttggtcttcccaat, ggcatagtaacttccc,
caggagtggggggggt, ttgggggcttggggcg, tcccccctttgaagag, tcgggggggggacttt,
tgtgggggcaatgttt, aatgttgtcatctaac, ggcatcacgttacctt, ggggggggcattatgg,
acttgtctctaatcca, gactcagtattgcatg, gccatcgcgtggtgac, gaacccctttgaagtt,
tggggttgagggcgta, gacggccggcccctca, ctgtgtcaggcatcta, gttgagtgcttcttcc,
actggaaacacaaacc, gactcctggctgcgct, ccttcgggcagcaata, tgtctgtacttggggc,
agctctctgtttatga, caatgctgagagaagc, gaggaaaagcgtagcg, tattgtgtaattttcg,
gacactaaagtagctc, tgtagtagaacttgct, ttgcctgaacgtccat, tccgagcaacgagaaa,
gattggcttaagaaat, atacgcgagcccaagt, aactcagcattacata, cggttaaaccccttct,
tgccaaagcgccttgg, tttaaggtctaccctg, ctctttttttgaaacg, taccatacaaggcaca,
tcccacaaaatttacc, atctaccccccccaca, ctctgctccactaagt, cctgttaaaactgtac,
tgatacaagttggctg, gttaatgattgtgtaa, tattttttttcggaaa, ctggggtctcccttcc,
gctattgcagctggcc, tattgatataaggttg, ttaagctacagtgtag, gatatacatttcattc,
agccttcaacctccaa, agtgtgttagccacca, accttccctacattgc, tctttcttatggttca,
ccatgcaggatcgtgt, cgctccgcgcccacgg, attacctttgcctata, tgctacgaagaaaaag,
tgtgcttgccggtcgt, tctgcgttatacaatt, gaccttaggtaagggg, atgacagcacttacaa,
cagagacatgataatc, cagtgggcctatcaat, ctattagtaagaacat, gagatgctggtaattg,
gtagctaccacgtgct, cgagttagactgtgtg, aaagcgtagcttgcag, aaacaacacctttgac,
aggggggggccagttc, cggcccgtcgccccgt, toccaagtgatagacc, aggtaaaaaaaatgcg,
aggctcttaaggcaga, tcctgtctggtcaaca, gttataccgtctctgg, agttattagggggtga,
atgccttactcaaata, aggtcacagctacact, gcaatcggtgatttgt, gagtcacatattgcag,
tgcactcttgactatt, gccccttacccccatt, acgatcattacataca, tacaaatcgtcacagc,
caggatgtatactgat, actaatacaagtaaac, acacttcctagtagaa, aaaataggactggact,
cttcttcagtttcgtc, cctgttaacgaagtca, ttagtactatgcttac, ggtggtgagcgggcct,
aactattgcgattgaa, tgtggctaatttttcg, gagagtctcgaagaat, taaggagatacttacc,
gctattagacaaaaag, gatggtaaaataaacg, ttgtgaggctccccac, gcgggactacatgcac,
tcttcatgatttagac, cagccaacatagtatg, ggtatcttggattaac, ttccgccactagatgg,
acgcccggccccgacg, cttatataacccattg, ctgactgaggtatgtt, atggttaggtcttagt,
tacaaatgtaacgtat, acagagttcgagctca, ttctaaccccagcctg, tgccacccgtgagacc,
cattgcgccggcgcag, ccctaaaaaaggtagt, gtaggcatgtagtgct, agtgaggggtttaaat,
agtagttgttagggat, aaaataacgaggatga, accgttcccggctaac, tttccatttcccgatg,
agttcagcctcagatg, tgttcgactgtctgtt, cccacctttcataggg, catcattgcttaattc,
aaaaggtgtactcagt, tgtactcaccgacaca, cgacagactctggtgt, gtacgtatttgtgcaa,
tcctcacaatcacaat, tagtcctagctccttt, acctgggggggggtct, ttcctacaagcaagca,
taacaacattaggtat, ggtggcggggatcttc, aagcagacggcactgt, tattatcttgactaat,
ctcccttcagacttgc, cgcgcgcgcggctggc, gaagggtgattcaact, acacggaggcacgggc,
tggttacacacgcctg, gagaggactcccgaga, ccacacggttagctcc, gcatggtataaagggc,
ggagacatagataaag, ttttagcatcgcagtg, gacacatgcataagcc, aaggttcccatatgga,
gcttgtctaggcaaga, gtcctgcctctttatc, tttgacagagtgtgac, gtgtcccccctagtga,
gagtatcagagtatat, gagtttgatggcctta, tctagccggggatctc, gatatctaatatttgg,
gttagctgtgtgtatc, agggattagacaataa, gtttgacaaaacattg, tgaaagttgcaccact,
tcaacccccgaaaaat, tcttaccctccataaa, aacatccagccgtaaa, ggggtaatacactcca,
gccatgaggtttaggt, ttatgagtggtgaatc, gtttttacctcacaag, gttgaggcgattctct,
tggtacctgaaagagt, ctgctaaagagctatt, gtctcactgtgctgcg, catttgcttcagtctg,
ttgtcccccctgctgc, aggggtagtttgagaa, taacgccgataaaaat, gtgcagcccgtccggt,
agatgttaatcccaga, tccttgttaacttgtg, acattcccgaattcag, gggatcagaggcacgt,
tgcttactattctcat, acggagaagtatttta, gttagcatgagaaaat, cttccaatacaacgta,
aacccacgtctccaaa, attttttcggtggaag, gcaagactggaatgtg, tgcttccccccctctt,
cacagtcctgttgtgg, tccgtacaatgtaccc, ttctcacccccgatac, ctctacaatccgaaat,
ccgaatttctgcctcc, aaatagcaaaagggtt, caaatgccggagtaga, gtaatctcacagggta,
tcaggaatttgattga, tgcctttttttaggtg, tgaggtcatgagtgcg, tccagttactcgcctg,
aacactgtagtcactg, tagaacacgaaggtgg, agccacgaatcttgtc, atagtgccaactcctt,
gggtttgcgttgctaa, gatattcagaaaagcg, gagaacacccgggtgg, cttcaatctattcgtg,
taataaccttcacgat, gaataccatcaaatgt, tgtgtttcacccatta, acgagtaggttatgaa,
agcgtcactgatctta, gtgcgctttttttatt, gatggtaccaagtctt, ggggggggcattcatc,
tactaaaaaaaggcgg, acgccgtagtcggcgt, aactagactatgacaa, gtgctgcgttgcgcac,
tctatctagacccctc, atgaaaactttcgtag, cagttatcgacaaggg, gatatcttaaacagac,
agcactgcagtacgtt, gaacataaagagacga, accttagaggtgctga, cagctgtacgaaaaat,
gcggagagaaattaca, atctgagctagcacag, acgttggcccgactgg, agtcaacactgatcct,
cacgtgcttgagagga, aatcgctaaaaaaatc, ctgggatcgaagccgg, atgatatgtaccttta,
acctggcgtagtggtg, ggaggacctgatattc, cagggggttttttatt, tgaactgaattggcta,
tcccagtacccggggc, accccctcccacgctc, aggaaatttgcgcaga, ctctgagtgaatcccc,
tgtaaatcgtgtgctg, caggcggtccgcgcga, ctgatcaagtccggca, ctcaggcccccgggct,
ggggggattgagctgt, ctctattagggactaa, gatattggcacctcta, aggcttaatgctgagg,
ggggttcttactaggc, agaagaatcttttacg, gcaccccatgcccata, taaaacttcacggtga,
accacattggtattag, ggattcttgtctgaca, cgatggcctttacttt, ctcatgaataccccga,
acgctgggctctctac, aaaacaaagtcgcata, agttaaactacgtctc, tcccccgaaatttctc,
tataatttattacgct, gtttcgctccgcagcc, agcgtatactaccacg, tgagccaggggccgga,
tacacgttaggactct, agcttaagactagcca, acccacacccctgatc, gatttacattgagcct,
gtgcagccttctgggt, tccgtctgaaaagaca, tctctatctccccccc, cactgtgtaccgtctg,
cggccttggaacgagg, cgccacaagtatgtcc, gtgtgtggcttgaagt, gtctgacattgtgcat,
tctacatatagctctc, attcccaagtccaggc, ctagttctatactgct, gacagtggctcatgta,
atgactccccgcattg, ttagatcaaggtttac, gtcttttttttagact, gccacagaaggtgcct,
ttacttgtaggtgtcc, ccctaagggggcagat, gagctcattcaagatt, cgttaatagtgctcag,
gctgtatcatcaacaa, gcacgaagagccagtc, agagtgggggtttttt, gtatcgtaaaaaaaat,
gggacttgaacaatgg, actgagctctcgtctt, ggcagttttttttggt, agttgcttttatcagg,
cgtggcccgaggcaga, catttagatacgcccc, ggcgtgcacgttacta, attacttcactgtggt,
cacgaaaaaaaccccc, gggttaaaaactgcaa, tgcagtttatgtgctt, gttttgtatccttgta,
agacttgagggtcgca, gttaagggtatccaga, gttcgtactcagatct, atctgatgaaacggat,
atagttacagtgtaag, tctgagccaagtatta, tgacctacgaggtatt, tctgtactccccaata,
tcacacctgctaactc, gtagttataggttaac, tgctggcacaagttga, acccctatgagatgtg,
tcgatttaaagctggg, agacaggatcagcacg, cctccgggagggtgag, gtcatgcgccatcaca,
cctgaaatatctgagg, actctgagcttccgag, gtccaacgttttctat, aagtgccatgtaggag,
gcacttcgtcagggtt, aggtgctctgtaaccg, gcgttcagaatctctc, gattaaaaacgacaaa,
cacccgcggtgccttc, gggttagaccccatct, gaactcatgaggattc, ctgctaggccagttta,
cttcgggtttgttcct, gcctttagagacgaca, taccccctoccacgct, ggagcgctgagggaga,
tcaattcttgcgtctt, gcccctatctcaaaag, gataatgtacttgatc, cgctgaaggattggcc,
tccccattcagggggc, aagacaagagtcatcc, cagtcttggtccttct, cattaggctgcctggg,
aacaatttgcataggg, acacctgctacggcaa, tcgtgatagacttagc, aatggaaaaaaacggg,
ggcttagagggagcgt, gttacggggcagatgt, gcccgcgctaggcagg, gaaacttgttccctga,
gtcatcgccacagcta, cttccccagcggccgc, cataagtctgccactt, ggcattcggccagtcc,
gtgtgatcccctcgct, tgatgacctcaagatg, aacgtgaaattagtta, cattctaaccgatatg,
ctgtcgcaggctccat, atctttatctatgctg, agcaatcctatcccct, ttattgaaggacagcg,
gggacggcactcacac, gccatgtaatgttacc, ctgtaagggaagccaa, aaccttccccattaga,
acgacacgtgcacata, taaatcctoggacccc, tgaagcagttaatcat, atagtgaagtacctct,
gctgacactatttatt, tctatatgacacagca, ctaccaggtttctttt, atcttttagaacctga,
actccaatgacttgcg, tatttctaagagggat, gcgagaaggcagaaca, tcctggagcatatagg,
gtcttggtttcttagg, accatacaaggcacag, caaactgagtattaca, gcttatcagctgcctc,
gaaggtgcaagcctga, tgagagatacttctag, catgtgctgccaattt, tgggtatctgttgctc,
ctgttatacattactc, aggtacttcttaactc, ttagagacactattgg, gggtctaatgacatgg,
gaatgaagcctgccct, cagacgccttgcgttc, gaaaggagaccttttg, aaatggactctccccc,
agcttgcctcttaaat, cactccctttttttgc, tggcccacatgattcc, agtcactgtgcttacc,
cttgtaagcagttttt, aattcgaagaataatt, ggctgggatcgaagcc, taaagcggactgtaca,
gacaagcacctgatgt, acggtaaccgattaga, ttttacggattttgct, tccaacggaacacagt,
aagaaaatcggaaata, agttgcaatagtaaga, atgtgattgtaagcca, cactccacggggggtg,
aagaactagttaaaac, ctaataatggtctgac, gcgggcatctatgtca, ctgcagtgtcgcaatc,
cctgagctcgggggcg, gataaaatgtcggaaa, cagtacagatggagtc, aatatgttctgtacta,
gacgcaaaactaaata, tatattctccagcgca, gctcgggggggggccc, cttcgtttttctggat,
acgccctgagatcgtg, gcagtctcgaacaaag, ccactaatacaccagt, tgagcacttcaatcat,
atatttttacaccgtt, agttgctgaatgaact, agaggctcccgtgggt, catcttatgcaactac,
cgggggcatggaggtg, catagaggttagataa, tcgatattctgatagc, taaaaatggttaccgc,
gatgttcagaggaatc, ctcccgcctgtgctcc, ggacagctgatacttg, ctagatggtatgttgt,
cttgggcatcatagct, gcgctaggcggctgcg, acaataacttggtggg, gccatcgtttgctgag,
gcgcattgcgccggcg, aatggaccactttctg, acagatttccggtgct, tgttggggggggaatg,
cacgagacagcgggag, tctacaaagagcccag, gaaatgccaactttca, aggaccataactggga,
ttcacggtgaaggaca, ataaacgaaatactgt, ttgcctgaaaagggat, tatgtagtcaggagga,
aagtctataaggactt, agctggatgcttattt, acccatgcaacctgca, ggggttccgccccccg,
tcaactaatttatggt, aagtgggctgagtcta, gtacgtggttctgaat, caggtgctgttgtctt,
ccgattggtccttttg, gattatacgtgtctct, tctaaccatctagtga, acagtaacctgcctgg,
aagacttatggttaga, ctattgccagaatgca, tcactacccttagctc, gggggggaaatgtgcg,
cctggtttccggatgc, tgtttatgttcttcta, atccctacattggttt, gtgcactggtcttcag,
tggatggcaaagcacc, ttatccctggtgtggt, ctagcacaatgtatgc, ccgctcagcattccag,
ataccccactcttccc, gggtgcgagtggtgtc, gtgccacgatggtcgg, ctctcataattccaga,
gtgcgactgtacttct, cttaaccacaccctga, acagcgtgtaacagta, tgttttttttacaccc,
caaacccaagtcttaa, cccttctactccggtc, tgggtgatgcaatatc, ggagacagatttccgg,
ctggatggtggttctt, actgtgcttatctata, ttaacccccccatgct, aaatttccgtgaaacg,
ttttttcggtggaaga, agcttatcttcccctt, agttctacattgtcaa, cggggtggacttcgct,
tgcaggcaatacccta, acttgagcccgatagg, cttccctcactgatgg, agcctctaagtcagag,
aaatgccccccctcaa, ttatggggggggcatg, tgtctggctgatgcct, gagcctgcttcacatc,
aagctgaattcattgt, tcgagtctcctttatt, cggagaaccttgttct, taacagtgagatactg,
ccggagcagcgtcttt, caaaatagttagactc, agtccggtgagaactg, cagttcgccaagatcg,
aattgagattagtgca, tgtaaagtttaggtac, agctacgccttctggg, cgggttatgagtgcat,
tggcctacattcatga, accaaacgacaagtcc, cactcctgtaagggct, cttcttaccaccctcg,
ccgggaccccagatat, aggacattagcctaat, cctcctctaagattca, gatggtccacgctccc,
tgatccttgatgaact, attacttaactgtggt, ggtttttttatcactt, gcttatgaatgtcata,
actgccaccatgcagt, catgccgtttaaaaac, ctgagctgttgcaacc, accagagcaatgccgc,
actttaatgcccccct, tacactaatcctacag, gagaccaataagttgg, caatttttgggatctc,
agcgatgctgtttcac, taccactctcagtgaa, ggtgatcgccctcaga, atctcgttgggtttgt,
cacagtggcgcgctcc, cacagcgttttttttg, tgtgcccagtagagtg, gatagatgcagagcat,
acttcttgggattagt, aggtcaaccttgccaa, tcaggcaacctattaa, aacgaagtggatatac,
cagcaacgaattatgt, cttatgtagtcaggag, ttgggaacgaggggtc, tgccacctgaagcagg,
caaaccgaaccagaaa, ccaggttaagtgggtc, ctttatgcaaactagt, agctaggcggggcttc,
tatatcaggtgtttca, gctgaggaaggattga, ctgaagatgctcgtga, acaactccccaaaggc,
gtcccagcattttctg, cttatgggtccacagg, tagatcagatcctttg, gttgtaaccccccctc,
gatgactggagtctat, acaactactctaggtg, tctttatttccccccg, gtgtaggacctcccct,
ccaccatactatgtcg, ttttaacactccttgc, tttggggggggagtgc, ctgtgcttagctttga,
caactcaggccaacat, aattgggggggtgagg, ggtggattcttttagt, attggtgttgcttgat,
cttgtgaataacctat, aatgactttaagaact, tggccagcaagtggtt, atccagtacatagtga,
gcgcaattttatttct, ctatatctatcagaac, cccagctttatgcccc, gtatccattcctgggt,
ttgaattttgcagagt, gttaacccccccatgc, aggatgtcctgtctcg, gatgcatatcatggtg,
gtggataacgaaattg, agccaagctaggctta, acttgaggcaccgcgc, ggatagtggaagtgta,
cgatttgaaattttat, ctctgatctaaacgcg, attcactagccactgc, ttgagacggtctgact,
agcccggttcccttca, acatcataagatacaa, ccaggcggtccgcgcg, tgagacctccaaaggt,
cttgtcttgggttatc, cgcgttgcggcgaggg, aacggtaacaaacaaa, tgtatccattcctggg,
tctaaatattcctaac, gaatggcgttatcctg, ttgagcttaaaaccac, tttgagatcagcatac,
ctgttttaaatgagcg, tccccactctaaactt, gcttttagcctcagac, agccccccccactttg,
tcccccatatgaagaa, gtgcttgcgcaggggc, ccgctagcgcgggtgg, cagaggctgactaatt,
cagatttctagtggaa, ttattctaggttaccc, caaccctctgccgggc, ctgtctggcattaaca,
agaagtgtttgagtat, tacttgagagtgtgag, aagcaaaacgaaagac, agggggggctcactat,
agctgccgacccctcc, gcccggcgctgagggg, aacgtgacatcttatg, ctaacccatccctgta,
tccccccccccagaaa, ggccggagtagggaca, caacgttgtttaaaat, aattccaggcacaccg,
atgtgactttcagctt, ccctaagggggtgcag, cagtgcttgcgcaggg, ggaggcaggggggggt,
ttttgtccggagaata, gttcaggtgtatctaa, gggtcctctgaggtta, cctttgtgcgccggtc,
acgactggtgctgaag, tggtatgaagttgagc, atcactgcataatgac, ttgcttactaaagcag,
tttgactagggtcacc, tgaggtacgggcggat, gggttcttactaggca, catagtattacactga,
tatacaaaaaaaggcg, accatctcagcagaga, gtctatttcgagaaag, acgccccttgatattt,
tttagtcctgtactcc, caggggggagctgaga, aaagatctgtgcggct, ggttatttctatctgc,
tctcgctgttctcacc, gtcacacataagctcg, gggaagagtcgctcct, gggttgtattctgtac,
tactagtcaggatatt, acatctaacataacac, catgattggaggcacg, ggctgtggtgctacca,
gtccagtgattcggga, caatacgcgttcatta, ggtctagaagtgattc, ttggtagcataaatgg,
gacaggtctttactgg, cgcgtcctctgtctat, tgggggggggctatgg, cagatggccggagtag,
tgataaaaaaatagcc, ggttcattctatggag, gcactgcagtacgtta, taacgtactttaattt,
tctcaaaccctggaag, aggaggagtagctacc, tttctaaccccccggt, gattacattcactagc,
caacactcacgtgatg, cgtggtttttttgtat, ctggtgttgggtctta, ccgagcctaagctgga,
gttcgggtcccggtgt, ctgtgcttgccggtcg, ctttggtcagcgactc, cttaattgtaagggac,
gtgaacgcaaagacag, tcccggtgttcgggtc, cagaaccactaaaatg, ctaaaagggttaaagg,
caattgcaaagagtgc, tgtctacagcctatat, cccacttctgaggggc, ccatatgtcgaaaact,
ccgtgactctctcgac, accgctgtttctaatg, gtaagcctacccccca, catctagagtaattac,
ttactccttcgtctaa, cccctgatctttatct, atcactatgtggaaac, tctacattttggcgag,
tgactaaaaaaagtgc, cttgaaatttaaccca, tacaaaaaaaacggtg, gattttggcccctaag,
agccccttacccactt, attgcaaccccagaga, tcccctacttccccct, ccacggctcagtgccc,
gtccgtcacccatttc, aaacctcgcactctgg, tctaggttatagcaag, ctggggcgcccgagcc,
gctgaagtggtcttct, ctgctccaggcaaagc, ctgagtactccttact, tgggaaaaggttatcc,
aagaaattcagcggtg, caggtccgtcactgac, tacccaagggttagga, ttcgagtgttattata,
gaggcccctggctatg, acaaaagatcgttaaa, ttcctccgggtcttga, acgagtgacctgtgac,
gaactgttatctgcct, tcgttaaaaaattacc, attgatcatatgaata, cgcagatgaggtacgg,
gcggtttccccctgtt, gcaggaataagtcatg, tgttgctcctagggaa, gctattctcctgcttg,
gcctgaaaaacacggt, ggaatgagtcttttta, gtccccccccaccttc, acaggtatggggggaa,
cagccctttttttaac, ctaggacaggattgag, cggtggggctaggctg, ggcttatgaagaagca,
gagatggtccaaatac, aacgccccacatgtcg, tactgcccagttatta, gtgtccataacttacc,
aggtacgggcggatga, accagtgccggaacag, caactacggctgcatt, ttgaggggggggacta,
ttgtagatacaatgtc, ctccagtttgggccgg, ggagggttgtggtatg, ggatggaaatatgcag,
ctctatggtctttgct, tagctacagcttaata, gtactgtttctggact, ttaagcgctctttcct,
attgtacagggctaat, ctggtgacccgagcac, tgaccaccacttgatc, tcgcacagcaatacac,
ggcctataaaaaaagt, tacgtctggaataatt, tttactcacaatctac, aacgcaggaggtgtag,
tatatcccactgtgcc, cggtgccttccacctg, catgccaaacagtgca, ggacccaaagaatatg,
ataatggtctgacgct, tgaaagtctaccactt, aggtagtctgagtgtg, ttctccagttgcgtta,
gcctcactgcgacgtc, tagtctttgtgcttac, tatctaaactgattag, tcctgttatctacttc,
ctttccctgaaaagtt, gagcgagcaactgtag, cttgcccagccgacag, aacacagttagactgt,
ctcaacctttcttagc, gactcccctcgaactg, ctgagctcgggggcga, tgtgaataatggtgtc,
cctgactcctttcgct, ctgtaaacgtcttttc, catgtggcccctaagg, acttacggagcatatg,
cccctgagaaccctct, agccatgccccgtgta, ttttcatcctgggagg, gctcacctgtgggcta,
cagttgtagtatacaa, gctctggtctttagaa, gggttcattcatgtcc, gccgtctcaggctctg,
tcgtttgtacttatgt, tggcgaccctaaggct, tagctcgaaggaagcc, aggcctttatcaaaag,
gacgcccccccccatc, gagctcctcggctccg, cagggtgtactgggcc, cttctcatactagtgc,
cacggtgatctcacat, tacttggtaaagcata, cttactgagagcggcc, tattgtttcacacacg,
ccagcgcattaccgtg, acgtgggaagtgcccc, atttcgatcatatttt, gtgactctctcgactg,
tggcactgctttcaca, tggttactaaaggcag, ttcagcgcattcagaa, ccgtctttgctaaaat,
ctccgtctagggacaa, attgcccccccccgac, gcgattgtaggaacta, aattagcgcctgggag,
tagacatgcccccccc, gatgtttgtgagatct, taccaaatccacattc, attctgataatacgat,
cgtggttttttttcca, agtgatgggcaagcaa, gaacttctgacgttag, cacaacctacatttga,
ccctgaagtcttgaca, tcccccccccatgctt, gttccgcggccgtctc, ccattgtagcccacaa,
tttacccttgtatgct, cctttagagtgagact, aacccacgaaaaaaac, gccgcggtccccgtct,
tccaagaacaatctta, ggccagaatcctttac, acttacagaagttcat, tctatttcactcaggg,
aagaatacgtttgtgg, agatgtcctgtgttgt, ctatgccggccagttc, ttatgattaaatcgcc,
ccaacgattcacatga, ataatgcaaactgaga, agatgccctagggtac, tgtgttagatgtaggt,
gcacataatgtgcagg, ctatagcccagactga, ccgtctcagagacaaa, cagatgcctacagttc,
actgtgcggtctgggt, cagctattgatttgcg, tcctatactttaacta, agtggtggatagtctc,
ctattggccgacagaa, ggcggccgggaagagt, atgccgggggctccgc, tgtccgtcctattgaa,
caactgtcactccggt, ctcacccccgatacga, ggccctatggcccggg, atacgcttaacaacat,
gctctgaccgatttaa, gaattacgtgtgtccg, cttctttttgctttcg, agccgctttttttaaa,
gctagtctacttatga, ccttgaaatagtaacc, taatatgttcaaaacg, tgtcttcgtaattact,
gttgcgatctttcaac, tcaggtctgcacagac, tcctacccacagatga, taaggtgttgttgctt,
tatgagattaacctta, acgctgagaaaagtcg, gctgcagttgggatgc, cctcctctagttcaga,
ttagccacccgtgtag, aactatgagcattcat, tcttttttgtaccccg, ttagcgacaaaaaaac,
aggaataatagtgtcc, ggcagtgagcaccgtg, cttaatccggagatct, gcgcactattgctggt,
ttgttattctgatgtc, acaagtacacattctg, cctcgttatccgctcg, ctccgtggctgcgcag,
tggagcttggagcata, gtatgtctcttgcgat, gtcagtaaattgttag, ataactttaatcctgg,
ttgccagcgtccgcca, caggtattcagctcac, acacctgccaccttaa, agtttgcatgttgcct,
tgggcattagtgattg, cagtcgtggtgtctct, tagtagactgtgcggt, acgttctcatcttctt,
cgggcgcattgcgccg, ttattgacttcgtcaa, ccctcggacctgtttt, cttcattggtcactta,
gtcattgaggctgcca, ctgtcagatggccgga, catgcaccaaaagtgt, atgctgtgctatcagg,
gttaaaaaaaagctgc, tggaatcaacatggac, ggcctgtgtgaacttt, aatgctacttattaga,
gaccgagggcactagc, ctttgttacttagtgt, caacacttctttactc, attttctggacgctta,
agatgtgataatcact, aaaagcttgctgattg, agtgcatacagagatg, ccacctgctggatagg,
ggggggggggagtttc, atgtgttaacgcatgt, ctgctcgcggcgcttg, gtagactgtgggatac,
tggactcctggtgatc, ggcggggggggcgtta, aacctcaatactgagg, cttaacattatgaaca,
actcataagggggaga, ggtgtgatgacatgac, acaacatggctgaggg, caagatcccggcatcc,
tgccacgatggtcggc, gcccatattctaagtt, tcagcgcttctctgaa, ttgcttcattggtcac,
ctcccttgcccccctg, gtctctctgtgagcat, ttttgtggtcaaacca, atggctggtccaatat,
tccttaatctcacacc, gaaaacggggaaataa, tgcggctgggctcggt, cacaagggggggagag,
gtgtaactagtttcca, caccacattacaagtt, cacccctatgagatgt, atgctgagacgatgta,
ttcccccgatggatgc, atctatggtgacatgc, aaccagtttgctagag, gcaggattcctcttga,
atttaaaacggagagt, accagttttttttggg, ttacacctgtattgcc, gagctaggctccgcga,
ggaaccactagcacct, aacaaaccaagccatt, ttttatagcaggggga, catcggggatcctcag,
cctgacgtggttgtgg, gcatgggtcaaaactt, tacaggtgagagtttg, ggaatatgctttcaca,
tttctccggtattatc, gttggttccttacaaa, gttgagaggggacagg, atagactaactgtctg,
ataacgtcattaataa, ttggtactgtgccatc, gatacgtgataaaaaa, cttcctgttgattagc,
agcatagggatccttc, acattcggaccctgaa, ttggccttgtgaattc, tagcccggctatgccg,
aattaaacgcctctaa, atcaatgatgacctga, tagatcttactagaag, cagactgtgccaacag,
ggccgcggggtactct, gcctccctcgccttta, ccttatggcttacgaa, gagctacgctgcacag,
acggtagtccagtaga, atagcgctccccccca, aatctcaccccacaat, ccacgcatcttaaacc,
actcgagaggtccaga, gcagttgtccatgtcg, ggaactgttgcctagt, tcaaaacacaacgatg,
caatctgggggggggc, aactggcgaccctaag, gtacacattcccgaat, gggtgacatagttaga,
gttaaccaccattcct, aacaaatcccaccgga, gacatgcttcggtgtg, acagttgcatgcctgt,
ggtgagttgactcaca, tcagtcccctgcttac, ggacccccccagcagg, cttaccctagaactga,
ctcattcctcgccgcc, caccttgtgccccgtc, ttcagctccttgacaa, gttggatcctgccggt,
atcaatattcgtttgt, ggtgttaatatgtttc, cgtgttttttttcaat, gtggggggggggcgta,
attagtacgtttttgt, ctggcaactatgctac, gattagggcggaacaa, ttgcgtctttatagca,
agcgtaaaaaaataca, attctatagctgatgg, taccagtatctcggca, gacggggctgagctca,
cctgagaggccgccgg, gccatccagtcagagg, ttacatctctagagct, ggtgccggtgtgaggc,
ttcaggcaacctatta, gacctcgtttataaaa, tgacacaggcgtgtgt, ccgcctgctcacactt,
ttaagccacggcccaa, gcgttatacaattttt, aaatctgggacctggg, acttacaaaacggaag,
agcgtgccagagggcg, ttgaattgtcatacaa, agtgtggtgaatcctg, gcatccccccacaggg,
ctgggggagataatga, gtgactgatgcctatg, gacatagctcccagag, tctatactggagaatc,
gggcgctaggcggctg, aatgaggtgtggcttc, taaatggcggggggga, tctgagcatgcattag,
ggatggttagtttaga, gaactccctattggct, ttcctctgtgcgcctg, aaatgaatccccccat,
tccatctaggccgggc, atgagtcagagtgcct, tctttacgggggaaat, agggatatttccttgt,
ctccctgaggtagacc, acccgcctgatgctgg, tagtctccatacccgg, cctaaatgcttattga,
atggcatacacacctt, gcaccatgacggctta, aggcctggcttttttc, tccctgactcctttcg,
gtcttatgtagtcagg, tttaccacagatcagc, ggcggccatgcttcgc, cgcggtgccttccacc,
caggtaatgtacccac, ggattgttagagtaga, cgcagggtggagtaca, tgagagatgttgtatc,
cttctgcctgtgcggt, tcatcgccacagctaa, gcttcttttttacact, gggtgcgagctgcttc,
aacgctttcattctgt, cacgtataaaaaaaag, actaggctgccgctag, cgcattcatgctctgg,
cctaaaaaaggtagtc, tcaatgtatcaatgga, gtgggtatctatcccg, gcactcttatgacacc,
cccagctgtacgaaaa, caccttttgcaatgat, tccccccccatatgct, cgagccttgcccatcg,
ctaaaatagaggagac, gggtagttttgtctat, gctccaatatacaccg, aagttttctttggtcg,
catagtgccagtctcc, gggcatggatctgaga, agagcttctcagtgtc, gtctgagccacaatga,
gatactttttttgtac, ggggttgggatactca, ttctaggccgaggcgg, tcgttttttttactag,
gcggagcgaggcgctc, cctgggggggcgtcta, gaccacgtctccgtgg, ccgcgcattccgcgct,
ctcgggcagcttaggc, ttaaatctcactactg, taatataaaaaaaacg, tccgaacttcctgctg,
tgtcaatagctagatg, ggaagtgacgatacgc, ggtagcagaggagtta, taccttaaagtgactg,
gtcggtgggggggatc, tacaattttgatggta, caacttcattagctga, ataccctctcagtacc,
atagatctaacagttt, cacgtccttttttttc, ccagtgagcatattga, atgttttttttgtgac,
ggggggaaatgtgcgc, tcccaccctgtataga, tgtgacctccactttc, tccccggaggtgaggc,
ctcacttcgtgggggt, tgtaaataaagatcca, aatgtttcgtattaga, aaagaacagtcgtttt,
cgcttaacaacatatt, tgccgctagcgcgggt, gcccttagtttaccac, cttccctcaacagtgg,
tgctgtgtcttgatag, ggtctctacaattatt, tcactgtctgactact, ctatgccccaactaag,
cctgtccattagcatg, tgagcccggggggggg, ctgggctacagccttg, gctttttttggggatc,
gtggggggggcatatc, ttgcgtctggagttcc, agatattctgactaag, atgacgggaagacaag,
cagcaaaaaaaaggcc, aagccagtcccattct, gagttcgatgtttggc, taccaccaaaaaaacg,
catagagctaagggga, ccctcaaggagaacat, tctgagatcctcctac, gccccagcctactaag,
cctaaaaaaacacgta, gtagccctcaggctgc, tgtatcccgggggatc, aatttaatctatgccc,
caactgactataaaga, gtcaaggtttttttga, tgtcacaactttgttt, ataggactgacactac,
atgtaactcgtgtaac, tgcctgatcttcttag, ttttgtaccaaatttg, aatgcttgtcctcaac,
taccaccagtgccgga, actctgtccctggtat, cagcatagttaaaccc, cttggcgaggcgcggt,
accaactcacttagct, atcctttggttccata, ttcgatcatagcaagt, gcaggccttcaattat,
tgccgcttactgtatt, atctggataccatgct, tgtcctactaggtgaa, ggaaaaaaaacgccag,
ctcaaacctgtataga, atgtcgacagccttaa, atgcgcaacacatcat, ctttttaaagggtagc,
gtaaaaaatgccacaa, ccgcgtcctctgtcta, catggccatggttagg, cttcagtttcgtctta,
ctcacgctgtgaggtt, cccggccctgacattt, acttccgcgcagtctc, ttgggagctcccagtt,
ttcttaaaaaaaacgc, gacctccccccgaaaa, caccatgacggcttac, ttataatgataacgct,
cgttatctgtggttaa, tcatcacacacttccc, actccgttccaactga, ttaatattaaccttgc,
ccttagctcagacaat, gcggggaaaaatgttt, gttgtcccccccccag, atgactccattaaaag,
accggggatgaccgta, tcgaccataaatgttt, aataatctacagctga, atgtgccaactttggc,
ttgatagctccagtgt, cccgaaggcaagatgg, acttaagggaaccttg, ataaacgcccttattt,
tgacggcatcctctgt, agtatgagcccataaa, gcttctcccagaattg, acttgaaatgttccag,
gaatgtcgcaaccttc, tatgtcttattggtgt, aacaccctaatctgcc, gttgttcatctgcccc,
ttattgcccatggata, ttagtagatcatcctg, atggtcaactactcat, attttogtaaaaacaa,
ccccgcagcgtgacca, gacatacaacaggggt, actacatatgccactt, ttagccttcaacctcc,
acatctcgaagctctc, cgcagcagctaacccc, ttgagttacgaaaatg, cattaggagacctccc,
tatttaaacggaaagc, gccctcttagcgctgc, aatctttgtgcagatt, tggagtgaaaacatac,
acatagttgagtggct, agtaatcgtaggaaac, ggtatttctgacaata, attacacagctgaggc,
ggggcattatggtctt, agttcaaaaatttcga, gaagatctcgtggccc, cttcgaacatgctgat,
cttaagggttcccatg, atcatggggggtatac, acaccggggatgaccg, catcaactggggatgc,
taccactgagaggtat, gctgcgttttttttat, ggtttggccatcccag, ctcgtctcctcatcca,
cattactgagtgagct, ggtagggggggggtca, caaatggccttggaaa, ggtccttttttttcat,
gaaaatcgagctgata, tatcctttcgcttaat, tgcttatgttatgaga, ggcggtggctgagatc,
cccctctaattttgtt, accaattcacttatta, atctcagaacgtagga, gatacttatgtggcta,
gtttatagtattgctg, ggactatacttaaagt, gttaaacccactgttg, ggaaagattaactaat,
tcagcagtaacacata, agggcctcgtgacttc, ctctcttaggctcagc, caagtttttccccctg,
cgaagttttttttgaa, caacgattcacatgag, aagatgctcgtgatag, ttatgcaggtgaacaa,
gtatgagtgcatcaga, cctcccccccccagtc, caaaagggtccccccc, catggaagataaggcc,
ccccacaccatgtggt, ggggccccccgaggta, tctgtaagccattggg, ggcggcttcctcgctg,
cccattattctacagg, tgtaggagtaacacct, tgcacgccgtagtcgg, tggtagcggccgcttc,
taaaacgtaacagaat, atcttaacgtcacgct, gacacctttgtaaagg, atcggttttagaagta,
ccggggccgggattgc, gtaatgagctgttaaa, tcttaccatattatga, ttccccgcttagcgca,
cgtcatcagacaagtt, aaaacatcctgttgag, attgcttaatgacgtc, tgcctttttctagacg,
actgacgtatatagaa, ttatgaacttagctgt, tttccccaccaacgat, tcacttgagcccgata,
cttactaaagcagata, tttcaaagagcacggg, ataatcgttggaatta, gcgcgagaggaccaac,
ctttcagtatatttgc, ttcatgggggggaagc, ttggttatgcggatga, gccacttgtttgggct,
gacctagaacttgatg, acccacatgacgggaa, cagggtaggccagacc, cttctatgtgagcagg,
tataattcttgagagc, gtattatgactgcagg, ttgtttcctacgtttt, ctccttacccaagcct,
acataagttccaaatc, accaaaaattgtctgt, agattctcccccccca, cacttgacatctattt,
cccgggggggcccgaa, gatggctccaactgtt, cagcccctgtcgacac, gctttaaaataggtgt,
gagacagcaagcagca, accaaactggtgttgg, tactgtgcagacgctc, gggagtgatttcgctt,
ctaatctactctgtaa, gggtatggtccccgcc, tctgtgtggacaaagc, acgagcttaagcaggg,
cccggatgtatacagt, gactagtctagacaac, ttcagtgtgtcagctt, agggagatcttaaggt,
aaagcccctatctcaa, aactttcgggtatttc, tggacatgccccaggt, tgagatgctccctatt,
gttcaaaaaaaaacga, ggcttcctcgctggcg, cccagttccaatcttc, tcccaaaaaaaatgct,
gaaaacgcaaataaag, tccttgccccccatta, gaatatccaagcctat, gaacacccgggggag ,
acagcgacagaagcaa, ctcgccttgagtgtta, acaccaccttccgcag, gagtaggttctggagg,
tacaatgatgggaacg, ggaaaagcgtagcgag, ccctacaagaatcact, tccccctggagctaga,
gatggaccgatttgct, aatacggctgaatagg, taggtctgccttgtgt, cagaagccggggggta,
gacactgtcaaacgta, gcagttgaaggctgat, atccctaccctaggga, ctttcccccccccatg,
tcaaatacgtgtttaa, ccgctaaaaaaatctg, gtgttcctatttggat, gtcgctttttttggta,
agatcctgctcgcctc, cctggatcttataagt, gtggcttagccatgat, tgttcctcgcgataca,
acccttgctactgcaa, aggtcaggtaaacaac, ttccttttgactcgaa, acgtgtgttcaggcgc,
gaaagacacttattac, ttaggcgcagcacctg, gacatgaaccacctct, cgtgcatcgtattaca,
caggccccttctagca, acaacggaatttgggg, ctgcatttgtgcaagc, aaaaacggacccaggc,
cagcatgagcaacgcc, gtggtgggcgctctta, ttcttcgccactatcc, ctgagtttagccttcc,
gcaagtttctaactgc, ccggcgcttttcagtt, gcttaggcacgagagg, tattataatggagtgt,
cccgcagcgtgaccag, aactgcttttttagga, ttttaactgggaatag, ggcatcctctgtgacg,
gaccaagacataagtg, atgagtgtcaggcgca, atgtgcgctagccaaa, gtcacaggggctactt,
ctcctctgatgattca, gacaattagcgcctgg, gcacctcttacacaac, ggaccgatttgcttaa,
ctgtgggggtctccgg, gttactgtgacagtac, tggccccccttactat, agatgggattccaccg,
gtcagtgtaagttgaa, gatggggtttaacgtg, tttcctcctgcgactg, aaccgctccagtctgc,
tgtaagcacttatctt, tacacagcatacggac, tgaccctgaagatata, cggactgtgctttaca,
ccccgaataaaccaat, ctgccatcccgagcat, aggaggtgategccct, cgcagattgttcctgc,
tttgatctggacttag, cggtccccagagctgg, gttaacccattaattt, gcggcttcctcgctgg,
acggcactcacacctc, gtaaagtcaatctact, ataggtaggaatgctc, tcccagttactcgtaa,
gaaaaaaatgcgcata, tgggagggggggggta, gagaatattaggtgtt, ttgtacgtggatattg,
attgttgttgtaagtc, gatcgttaaaatgtaa, ggttgtggtggcattc, catgttgcactttccc,
atttacttgtagccac, agacgagttatgaatt, cggagtagggacattg, aagtagttaattgaag,
gatcagggggagaaca, ctaatctcccactatg, gagatggggggggtgc, gtggctaatttttcgg,
ccaacgtcacatgaca, gcgcgggacttccagc, ccctagctccctgtat, caacttcaacaaggga,
ttagtcataggttccg, tcatacataagagctc, gagtttaccaagacta, aatttattacgctgag,
gtccggttcacttggg, gggggggtatcatgga, gatgggctgtggttac, gccacctcccattccg,
agtagtgaatggaact, caggacctcacgggga, gccggtgccccttggc, gagtagaagacactgg,
tcggtgggggggatct, actaaacttgggcaca, ggttttttttaacggg, tggcgcttgtctaagc,
accatttttagctaga, gacgtaacagatacac, caaattgagcagtctc, acaccgtcttatctgt,
gggacaaaaaagctag, ccggcacatcagagga, ggttaaactttgtctc, gggctacatagcagat,
aataagacggcatttt, agtctcctcttttaga, gcgcacggtgcgtaca, ttctagtccttcctgg,
ctgaatgaatctcatc, tacgctcaaaacatat, gtgtgcccctgcatta, cataggtgagtcttac,
actcctattgataacc, aagcctcgctgatcca, ctcggtttcggctgaa, gccatatgtcgaaaac,
ctaagttttagaacct, tgtttcacccattaac, gttgggatcataagca, acaaccttccccggtg,
ccactaatccattatg, cctcccaattccatgc, ccttgtttgagatttg, accaacaccaaagact,
taacacattggctgaa, ccttgttacccgaatg, aggacgtgaggagcgt, ccttcgaatgtccatt,
taaatatgtgaggggg, gtcttctttacccctg, cagtttgcgatattgg, caacgtagttagacct,
gtgcctgttgtcaaaa, acgaggtatttagagc, gtgtaaaaaaagtgct, atgggaacaccattat,
cagtgccggaacagtt, gttctgtagccacctc, ttggggggggtacatt, tgtttacgctgggcca,
ctccctggactccata, tctggtgaagtgtaat, caccaacgacaaacta, caatgtctgcatgctc,
tggaggtcttagcatg, agggatagtgtgtggt, ttcaaaaatttcgagt, tacataagtctgccac,
taaggttccaggaaca, accaccatactatgtc, tggccggctgtgggtc, tcatgctgtcgctttc,
taggcccccccgagcc, aacgattcacatgagt, cggtagttttttttgg, ctgctgacgtcagaca,
cagtgacacattgaag, ggaatggtgcattcct, ggtgagtgttattctg, cggcttagaggaagcg,
cccacttcaggagatc, acctccttaattcccg, attccccttagcagtt, atataacaggcgaagg,
cttggcctcctaggac, tattcactgacactag, ggtattgcttcgctga, atgtgaccacgcccac,
ctagatggtccacgct, ctaactcgtcaagtgg, tagcaaaaaaaaccct, ggacccccccatatat,
ggtgctggattacagt, acgcctgcgtgtacct, taaactaccccccccg, ttttcattatcgccag,
ttcgcttttttttatg, gaacccaaactatggg, tagccaaaaaaagtct, atcatccgatatgctc,
tgccgttttaaagaaa, tacataggtattagga, gggactgtaaacttta, cgcccgccttagctgg,
cccgaaaaaaaaccac, ctatctcccccgaaat, ctaaaaaaactagcat, ttacagaatctacctg,
agggtgtatatgtctc, cgcactgctgatgatg, tagattaactaaacag, gctgttaccacatctt,
cacattaaaacgtttt, gggagtgcagcattaa, tatacccagcacttcg, cgatggaaaaaaaaca,
cgatttgccttttttg, gactgagctctcgtct, tgcagtcgttctttcc, ttgcaagtttttttgc,
tgttagtactgccctc, gcgttcaaaagctgaa, gctcctattcatccca, agaggttctagcgatt,
aaaatctgttgttgcg, ttaaagtgggggtaag, ttaaattctaggatct, ttatcgtaaaaatcac,
aatacagacagccatt, attgctcatctcactc, attccggtgctctggg, agatatagccccccct,
gtcgtggtgtctctgc, gctcactttcttccgt, ttccaacttcctagaa, tgcatcgtgctgattc,
actctgttttttggac, gaacacaacctgtgac, acgtccgtcagcgtgt, tggacatttaaaccag,
tcctaggtaatgttag, catgcgcctcccgcta, gccggcgagttaatac, acggggcagatgtgag,
gaggaaagttagcctg, gaagggctagtgaaca, aactacaactccgaaa, ccgacagggcggggaa,
tgtgccgggcgaagct, agcgctcaatccctca

TABLE 8
BRCA predictive nullomers.

ttatgcataaacgtca, aaggtgaatggttgtt, cattttgttaaggacc, aacaagtagaccaagt, ggaaggccgctgaaaa,
ctagccagcccgagcc, tggtgtcctggtaggg, caacattttttccgta, caatgtcatattatag, cactgtaagaatcccc,
tttggtaatgaagttc, gggggagaacatattg, agatatgcgcagaaca, gcgggcgattcgcccg, tattcaacctaagcat,
ccggtagagacgctct, gcagtgcatcttagca, ggctaggacgtaatgt, ccttacttggctctgt, gcatcacatttttaag,
ccggaggcttaggtag, cccatatgcagaactt, tctttcgtttttgtgt, aacaagtagctgtagg, ctaccccatcagtcag,
ttttttccggttgttt, gcttattcatgatcag, tgaggcaactcaatag, tacaggggaccaccct, tttccgttgcttttgt,
ccaaggttaaaaatgg, atgcccttagcaacat, tcacaagagtaaagta, agtcgcaatcatggct, aaacgtcagcttgcaa,
atcacttcttgccatt, cagtggctctatgtcg, gcatggtatccatgct, atcctgtagctacata, ccgggtttcgagagaa,
catattaagggtcttt, acaagtgccagaacac, gggtgaggtgcccaga, tggagtccattgagag, ttccttcctgacgcag,
aggtgatagccactgc, ccagaacctatatccg, taacgcaaatacagag, tatagcttaggtattg, ggggctgcagttagac,
ttgaaggattcctaca, gaatcctaagcccgcc, tctgcaggtttaccca, gctatgattttagtag, tggccccttagaggat,
ttagcgagtctaccat, gtgtagggtcaggccc, ctatgagtgctgttaa, ctgtctgaactgtggg, cgaaaggggtttgccc,
gaggccaggtttattg, gatagttagcttagaa, gggctgcagttagaca, gcagagctgcgaggtc, agttcccaataggatg,
tggagaacaaggtctg, actccagcgttgacaa, ctggggtggttgcaaa, tgaacaatccttctca, cggaccctaatttagc,
ggtagcagtatctatt, tgaggcctttattacc, tgcacattcgtctcaa, cattttcgttaataag, agggaagcggagcccg,
ctagggatcccacaat, ctcaccgttgtaatcc, cattgatgtttatccg, tacgaacaccaaaata, ggacagactgcagtgt,
tatgcataaacgtcag, aatcccagcaccttta, gaatttcctggtatga, tgtgccctgtaaagga, agtgcaatctgcttgg,
gtaatttaccacttat, aaaattgaatatggcc, gtactttagagatccc, cgcaggggatggggtc, ataaccaaaatcagcg,
acgtaactctttagag, cagaaatgcttaggca, aatgcttccggtttct, tagtgacacagggatc, aaggccattgactgaa,
atattagctgctggga, ttgggcagcaggaccc, attcaatggcatgcat, ccaacattaactatct, ggtaatgcttgttgtt,
gagggaaactacacag, agctcttttgtgcacc, ggctccttagcactgc, ggatttgtacaagttt, ttgactttaggcagaa,
aaggaactctcgcatc, atagtagagtggtaca, attttctttagcgagt, acatataatctgcgtg, tataagttagagcatg,
attgatgtttatccgg, cactctagtatgttag, ggcccacctcacatat, taaacgtctcaaagaa, caaatggaccctaatt,
aattttttatagcgaa, gcacagtcggtaaggt, aggcttaaaatggggc, agacacttaaacaagg, cctttaagcggttttg,
ggcccacgtggggacg, gaggaatctcgttttc, caaaagcggaatctga, ctgtcagggtaatttc, cacagtttagaaaggt,
gaataccaatttgtca, aaacattgaagtgttg, accctttgaatccaca, atttatgcataaacgt, cctgaactcgattttt,
caaaaggtctgacggc, ttcggctcataatctg, tgtgaaaggttggtta, aatcctgaatgtattg, gtctttgtgtccaaac,
tgtctggttgccctta, taacttgtcattaagc, cactgcgagctccaca, tcagaacgtttgccta, gggcaagtaatacatg,
gacttgttaatatctg, gtgaggcaggtcattt, gccaacccggttaaac, gtccctgggctagccc, acctcagtttattagg,
agcacttgtgggggaa, taaaccacaaccaaca, ccatcttaggacttga, atcccctccgtaattt, ttttaggtacaatcaa,
gagggtctgtatattt, caaacatctgtggggt, gacaaggtttaaggca, atcaaaaggtctgacg, attcgtggacatagta,
cctggttgaggctgaa, ttagtacaggaattta, atagggacttgacagt, atgagcttagagaaga, ttcaacattttttccg,
ttttaaatagcgtttt, gagtaagacctgtcct, ccagattgcggcagta, cttttctttgagacgt, tgttgaagttttgagt,
ccacgtggggacgggg, acagtttgttcctcta, ttcaagcttacacaat, tttaattgaacgcaga, agggatcccacaatgt,
ggcaatgaacttagag, tatttcagtacccctt, tgaaataccataatgg, atgtgcacttaggcca, aaaagtaagccccagt,
catatatttgaccgtt, tgtgacaatagcttag, agcatctattaatcct, gccacatcttcagtat, aagagtgcagttcaca,
gcacctacccacagga, tttaggttctccctaa, tcgctcatctccatgt, ctatatccgcctctct, gctggccatttgatat,
actgggtgagtatggt, agtggaaggctcatac, agctgtgcgaccaaag, gtctggattggacctg, ccttctgggagatgtt,
ttcccaaactccttgg, aggctaggacgtaatg, attggtaatgtgtccc, taatcgaaaaaaacct, gcttccctaaaaccta,
tgtgctgcagtgtgtt, ttccggtttctgccca, tcgatgagcgctagag, cagttaaggtagggag, ttatgccagaccttct,
gaggtgagtcagaata, acaaacggcttatgtt, gttccagggaggctac, gactttctggagtacc, aaactgccatgtatga,
tgtggattatatgctt, cattgctgtacagtct, gattagatcttactat, taagtaagacacaaca, agcctagcaggtttta,
gatgaccttagtgtcc, caaggataatggtctg, aaccctttacaagaat, gttcttagacatcata, aagccgccaggaggtg,
ctgggaggtcagccac, gcttccggtttctgcc, gtgtcattatctctct, ggttaggtatttgaat, ctgttagtttggtaca,
cttaggtcgggagttg, ttgtatccaatccaag, cctttatcccgatttt, cgttatatttgtttgt, aggagtctgcctcacc,
gataagcttaatcaag, gtcccttcatcatagg, gggaagaggaatagac, gaagaacttaccgttt, gacgtaatgtctaaaa,
gaacaaggacacaact, ggcagcgtaagcctat, gagggagtgcgtgaat, ctttatgtgagaccct, cgtgcccagctctatg,
agagggaaactacaca, cagcaagcagtctggc, agcatagcttgagctg, gggatggggtccctag, ggttattttatgatca,
gacgagctgagataag, gtagattacttaagta, ttaccacagatgtcaa, agtacacttacacaac, gaagatataggactac,
gatagtagagtggtac, aaccggtagagacgct, tgagctggtcaccttt, tttatgttatctcgtt, gcaaaagctggggtgt,
ccgaattcatgtcatt, tctcagttacctcaac, cgtctacccgccagct, tgtccagtaatcaact, actgggcgatgcagat,
gggagttaataacata, tcatttctcaattgac, aaataagccccccccg, atcacttgtccatgag, ccgccagctcccagga,
gccctataagttgccc, ccatgttaagtacctg, gtattgtactaggagt, ggcttcacgactctga, ccactatcaataccca,
cttcaacctgcaactc, taccaaactacataca, tgttttttaccaccct, gaggccagcggtatga, ttaatccccaatcttt,
ggttgctcaacttaac, gaaggtgcatctcaga, gaggcgggcttatgcc, ggcaaagattgaatct, gtctgtactgtataaa,
aggcgggcttatgcct, aagggttagggaaggc, aagcctatttgtgtga, cccaggttggcgggga, gttagttccccatatg,
agaacttaccgtttct, gggctgtaacggagac, ggagatattgtgtatt, cacaagccttgggaat, gtagtgctcactgctg,
taaggagtagaattac, ggagccctatgtgttc, cccctaaccttatgta, ctgacaaggtttaagg, tcccctccctcgcatg,
agtcttcactgcattc, gagtaccctgcatgat, gaatctcagttacatg, ccattaaaagcctgat, aacttaaagtgtggat,
agaggttaactcaaga, gaccagaggagtgagt, cgttttttttgtgcca, cgtatctgtgatgaaa, caacccggttaaaccc,
aaaagcggaatctgaa, ggcccaggttggcggg, gcaggcgctattctga, agttagactccgttca, tatatcaagagctgta,
tgccaataatctgttt, acaccgcatcatgaag, ccactagagatggtaa, cttcccatgcgcccag, atagcttaggtattgt,
tgtgacaatcatggat, cctaaaaccatcccgc, cttaggaacagtgtcc, cagggtttgtgcaacc, ctatgtctatacatgg,
tgcataacattaacca, cctactgggagtgttg, ctcaaatcagtgtctg, gtgtaccaggctggct, gggctttttgctatgg,
ttgtctgttatgattc, ctaccttagagttgtg, gagctggtaatagata, ggggattagagctagg, ttaatgctccaccact,
taatcctttttcttgc, gcaaaaaaagctcctg, agattccccatttcaa, agatggcgtatttcac, tatatgttacgagttc,
ctagtatgggagaaag, tcgatttttgcaaaac, gttagactccgttcaa, aaatcctcatgttccc, gccttacatagaatag,
aaaccacgtgttacct, ttgattttcactagaa, tagctgctgggatctg, ctagtcttacaatgtt, attactctgtcatgca,
gatgtatcctttcccc, aggcacctcagaacgt, acattgaacttatatt, tgagagggtcacaatc, atactgtggaacttaa,
cgctattctgagccct, ggacctgactattaca, agaacgtttgcctatt, gggcctgctaatgttg, cagccccggtgcggta,
ttgtcagtgcacaccc, gttgcaagccacttaa, ctgtaggccaaaacaa, cccaaggcattatagg, tggccttatggttcct,
aacggaatgttagcaa, gcttaaccactttata, ctattattgaggtcct, tggaagctcaaacttg, accagaaggttaatac,
ggctgaagcggtgcag, cgtctgtgcccggctg, ttttggactcgtgaca, tcaaagggcttttagc, ttagagcgcttacatt,
gtcggtaaggttcaga, gtgtaaaatgatctgg, aacctagttcaagatc, ccagcatggttaaaac, aatggtacctaactaa,
tagatcttcgatccct, ccaattcgttttacag, ctcgaactgttaaact, ccctgcccatattggg, ctctaatgcaacttat,
cggctccacttttaat, cattccggagctggag, tcttggatctataaaa, tgactgggacttgact, ttctcgtgatagagac,
acggagcaatccaaca, tttctcttttaactcg, cgtcaaattcattccc, ttatctatgttcgtgg, cacttgcaaccacgcc,
ccagggggcaaaaact, ggaaaacgagagcaat, gggggtcatacttatt, tgtatggttagagagg, aatatgttggatacag,
taccctttgaatccac, tgcatataggcaaaag, ccctgtttgggaaggg, gaagctcacttagact, ggtcaagcaatttacc,
tgcgatattttttttg, gcactagaccaatagt, taccgggtttcgagag, ctccatgtctttaggc, agatttatggccttgc,
agctgccaacttatgc, tgaataaacctcgcta, gctacttgtatgcaag, gacaatgcttgagcca, gcacctcagaacgttt,
gtcttctggagtgagg, atgcatgttataccgt, ccaataagccggtcca, gcttactgtagcccta, agaggagtggtagctt,
gagcaggtatgcaaca, aatcgtaagcactgag, agtgggtaagttagca, agctattgcctgagag, gatgggtttcatgtgc,
cttccggtttctgccc, ccaattgcagtctctg, gttatgtcccatcttt, ccatgtggtggctgac, gagagtaatttggaac,
gaaatagctacacata, ttggtacctgaacaag, ctaagcagacttgtac, ccagacccgcagttaa, catgcagagggacacg,
ataactgatcggcagg, gcttccccgttccctc, cttcctgtttgaatta, gtccattgctgaactc, agtcggtaaggttcag,
cttctgacttcgttag, ggttaaaccccgtcgc, tcggtaaggttcagat, catgagttatacatag, gagccttgggatcttt,
acataatagactgtag, cccgaattcatgtcat, cagagtcaatgtataa, gcattccctgtttagt, tccacaaggtgctgag,
ggtaatagcaccgaac, actccctgcctcggta, ctgaatccttgcaatt, cccagtatggtccacg, gtacgtctttcataga,
ctactttttgaatcct, cgtacgatcatcctgg, ccgaagacctagctca, tagtaagtttgcacca, accgctcccggctata,
cagaaaacccgaaaca, aggatatttgcagatc, ctgatagcttggaagc, gccatgctgaagatta, ataaccagatgtccat,
ctgcttaactccgtaa, aatactggctgtgcgt, cacatctctataatgt, ttgccttaaaaaccca, gaactctcgcatcctt,
aattttatgcgagaaa, gccctaggcattatgc, catcgtaaggaggact, aaacatcgctaattct, ctgaactcgatttttg,
tgtgggctgtaacgga, tgaacatctgcaagtc, atccctaatacatagt, ggaggcttaggccagc, tggcttaggtccttgg,
gggcaccatagacaag, gcgcgtgtgtctttat, gaggtgtggaggtata, accacctgatcatatg, tgatgactataaggta,
ttccccatgctttttg, taagctgtctcttggt, gtagtatattaaggtc, ggttgagcgtcagtgg, agcgaaattttacttt,
tgggggaataatgcta, agagccccagccttga, atttaagctgtaatct, gaaaagaacgataagc, tgacctagattaccaa,
agtcgccctgtgtcac, cttatgggtctttgaa, cctggaggcgtagctt, atcaggtatgtgcatt, tcgaactcgtggactg,
atgtttcgtgtcatgt, atgcggcaccaaaacc, ttttttcgttcagttg, accccacccaccgtgt, tatcactcctctatgc,
taaaacccccccccac, ttttgtgcaccctcgc, aaggacatgagtgttc, gacgggtgcatgtaat, tgacgagctgagataa,
cggtcttctggagtga, cagattctacacaaca, ggcttctttagtaaaa, gagctatgtctgtgtg, tacttcttcttgtcac,
gtatttttgactccac, catgatatttgcagtc, aggtgcaaggaactac, ttaattatctgccgac, cagcccactggcacat,
agccatcgtgcgaggc, gaacaagggagcttta, agtgttgtactgttgc, ctcggtgcctggataa, gttcggctcataatct,
gttggcgtgtgttaca, acactgcaactgaagg, gattatgctttgtagc, gagcttatgcacagaa, ccgagagagaaatctc,
tgaaaaactgaaccga, gtgcagttaacgcact, gagaagcatgatgtca, tatttccgttttgaac, cagggacattaagatc,
ctgttccttaccccag, ggcagaggatgtggca, atgcgtaaatatagta, cggctggaagttgggg, aatacaaaggtttttc,
aaaccggtagagacgc, ttgaactctcacgtct, aacaatggccgttgaa, cccccgccttccaaac, ggaccctaatttagca,
tcgtggaggtagttac, tcttaatgctccacca, taatagcatcaacgtc, taaatcctacatgtta, ttttggggtagtttcg,
cacctcagaacgtttg, tcttctgccaactggt, ctactcaggaacctta, gaaaatggaaccttac, gcatgtattatttcag,
aaatattctgtgtacg, caatattactctcaca, gccagtagacagacct, acatatggtaaacctg, gtataccactctcttt,
ggcacacgacttaaga, aaccatatcttgtagc, aaaatttgtggctgcc, ccctgccttctaagtc, cattgtcatggtagat,
tgcaggaatgcactct, tattagtaaggagttg, cagtatcaggcttagt, tctgagtactccacct, tcagcgattttaaaat,
atatcccttagggatc, gatgtttatccgggat, tcttaccccttaaatc, cttcagagtatgatgt, ccctagtaaaatagat,
tctaaatttagtgtcc, aatccactttcgttcc, atattcaggcaacacg, tggaatgcgttattta, agtagctaaaaaacga,
acgttagtgaaatatg, taggaggaggacatag, ttttttgttggtacgc, aggtgctaaaagtagt, gtgttgtactgttgcc,
ataatgattgtcgtaa, ttgcagagtgttacat, gcctggcatggcgaac, gtccattaacatacac, cgtggccaaaaacatg,
tatgttatctctatgc, ggagagaaatgcgagg, aacttagggtgcagca, cccatcctcaaaagta, cgcctcccgctttcac,
aaaagttcctggtatt, cttatcaacgtagcta, tagatatgatgcttag, aatagcgttttcttta, ggacttctaactgttg,
gcgggcagcaatactt, gcaaacgtacgatcat, ggaccatatgaagtgt, tcaaacaacaacctca, gaggttgccgttagac,
tagactgataatgtga, tccccacctactggga, aacctttaggtggtaa, aaagggttctgattat, cgtgggtcatggagga,
ggctcctttgaaagtc, cctaagcccgcccccc, tgtaacggagacttac, caaacgtacgatcatc, agtgctcttacgtata,
cttcctagggtaaact, gtgaggctggtagcaa, attctgtggcccattg, cctatatccgcctctc, aatgcgaaactgctga,
tgaaaaccggtagaga, tgacggctccggaggg, tacaccaggctcccca, tttctgtactcaccct, gtctgaacaaatgatt,
ggagccccatagaagc, gatgcattttagataa, gtctgggtccagactg, agcttaggtattgtac, cggtagagacgctctt,
ggttaaagggaacagc, ccttagccagctgatg, tcagagaccgaggaca, tataaggaactctcgc, tgctggaaatgcggac,
aacgttgctcagtaac, gctacctcaccttcca, cgaaaaaaaagtttgg, ggccactaaattttct, ctcttcaatagttagg,
gctggggctacagtcc, tctcgcatccttgaat, cttagggctgtgtaga, agaggtgtagagtaca, cctggccaacccggtt,
ttctagggtgtgaaat, ctaatccaataagccg, caaactgttgacctcg, tggccaacccggttaa, aaacaagtagctgtag,
ctaccgggtttcgaga, ttagacccagtgctat, ttccagtgaatagact, ttgtaaccaacgtatt, agaattctactggtga,
gagggatgaggtatta, gccagtgcagcttgcc, ccactgtcagacccct, gatatgctggcttagc, attagcccatgaggat,
gatgctgacagccttc, attccagtgaatagac, attggagtccattgag, ttaatggttacagcgt, ttgcaaatgaaaaccg,
ttttagttgtaacgtt, tctaccgggtttcgag, aaggtgcatctcagag, ctagcacttgtggggg, gcaggacttgtggttg,
ggctagtctgaacctc, ggagtccattgagagc, tgatagtagagtggta, cagagaccgaggacag, ggagaacattctgctc,
tcaggttccgagtcct, cttcgatccctttatt, actatgacattgctgg, aatactccctctttgt, cttgagaatgatatgt,
cagcctagcaggtttt, cggtttctgcccattc, cccgggtcgccgctgg, aagtgagtaccctgca, tgagagcggctttccc,
tcagtgacaacgaata, tgcctgttattgtctg, ttgtgcttcccataga, gccggtccataattat, gttctttctgcatgtc,
acatccgccgcccagg, aatttagcatccgagt, atccacatccctgaag, accaagcaaaccgaaa, tgtataaaggagaccc,
gcaatacaactgtatg, ggctgttgaagcttaa, aggtgcacctagagta, gtaaaatatcttaggc, atccatgctagtgata,
tgtagcagtctgcaaa, ttatgaatagttcact, tctgctctgtttccgt, ctaaaaacgctaattt, atatatttgaccgttt,
aaaaaagtcccagtgt, gattggtgggacaaat, cggatgcagcccctgt, tgaacagctacttgtg, atagaatggtgcatct,
ccgtaaaaattctgat, tagactctaggattaa, cagctcatagttgtct, tatctaccaataacac, gtgggtactccaagag,
gggcgattcgcccgcc, acatgtaagtggatgt, cctttatttatggtaa, tggttttataccctga, gcttaggtgggtttca,
aacaatgttagcttgc, gttcgggcccaggttg, cgctttcacgccattc, actgtactgtgagata, gaacaaatgattcaac,
agcaatacctttttaa, gggattactattgttt, ggtttgtgttacccag, gtaactatgatatctg, ctgtggtgtaatgtcc,
tgcttagcgtcccaaa, acgcagtcacctctca, ccggttatcccagtac, tataacaggtaagtcc, tcggctcataatctgt,
aagagaggatagttcc, ggtaaaaacaagaggc, tttactagctatctgt, ctataatattgtaaac, ggccccagagacctta,
tcccgtgcccacctct, tggctgaacttatttc, tgtctggtgtctaagg, ctgacggctccggagg, tgtgtcatagatggac,
atggaaaagtaccctc, catgcacagagcgcta, cataaagaccaacatg, tcgggaccctcctctg, ggcaattcctccggct,
tagacacaggcgcctg, ataaggaactctcgca, acggggtacattagat, tatctttcctgcactc, cccaggacgtgatgga,
ctgggtagagtgcagc, aagaacttaccgtttc, agatcccaaagcttca, ataaacgtcagcttgc, agcagaacaactactc,
tcaggcgggcgattcg, gagagttaagtaaagg, ggccaacccggttaaa, aggcttgtctgacaga, ggagtaaggcagaagg,
gaatattattgttgat, aatggagggggtgcta, aaaacggaacacaaaa, agttaggtagcagtat, ttcccttatcatatcc,
tgtgattgtacgtttc, taaacgaatctccaac, ttaaaccccgtcgcta, ttgaccctcgacgctg, atgacgaatgggaatc,
cagtttgattgccacg, tcactgtacgttaatg, agatgtgcaaacgtac, ggttgggttggatatt, ctaggatatagagttt,
ctctaccttaggttta, gaacgtttgcctattt, atgtgtgtcgagttcc, ttcacacaaacccctt, tatctactgttgacca,
ggttcctgagcagagt, tcactctgtatggccc, gcatagttttcagctc, cagaacgttttatccc, acgaagtcataaaatg,
gacaggggttctgttc, atgtcatgatcccccc, ttatgttatctcgttt, tttaacgaaaatcagc, catttttactaccgca,
cccagaacctatatcc, cattcttcttcctaag, agatgcccacattagc, tggcacgtggcaccag, ccttaaaagtgcaaat,
cggtgcctggataagc, gcactggatgcattga, cgttccgtttttattt, gtatcttctctaccgg, tttactcagcatgaat,
tcccacactattagtt, ccacatgtgcaatgag, attagacgtaaaagca, tacctttatgctcaat, agtctctactagagag,
gtttgtatatccaatc, gatttgagggaagtaa, cttttgtgcaccctcg, aaacctctatgcactt, tagtgcaaaggccctt,
aaggtgacttccaaac, acattatatgtcatcg, attaggcatccagccc, aagatgacaccgaaat, ttagcatccgagtgga,
tacagtggtgggttcc, agcaacaactcctctc, cttgggcggcttaggc, cacatcaatacgtaat, attaagcttgaaaagg,
aaagaacgtctgccct, ctaatctctatgccat, ttcatgagaccgaaaa, atacgtgcgactattc, acataggctaggacgt,
tccagactgtctgcca, catagggtcaggacag, actgaccgttgtgaaa, ccttttttttggggta, ctctaccgggtttcga,
ttcatggtagggtttc, cggggtacattagatg, gccaaacctgccaggt, agaatcctaagcccgc, attgccacattgagag,
tcataaccgaaggaat, ttctggcaactgatat, tttctttagcgagtct, tagtccacacagccag, ccggaacttttgtttt,
tgttcgggcccaggtt, tagccaactgtcttcc, actgaagattatcgat, cagaggctcatcattt, ctccaaccctattaga,
ggagtataagggttgg, tcctgtccatatgtgg, ccaagccttaggtatc, tatgatttcctgtggt, atattcgtatgtttgt,
ttatggataacattgt, gccccaaaatctttaa, tctggtggattagaac, ttttttctttgtgcgc, tacaatctgatgtctc,
ggtcaggccggtctga, aatgaaaaccggtaga, gtaagcataagaggac, tcccaattcctatcac, tagcaaaattcgtatg,
agggcatcataaaact, atgcccaacagttgga, agactacagtggtggg, gaaggcttcagctatt, catgctatgaggtgtt,
tctcttacgtaattgc, caatggccgttgaatc, ctataactctatgacg, atgttcctaagtattg, tatcacttcacacaac,
ttcgttatctcttaag, tttgggctctacaata, ctgagagcggctttcc, ttttctttagcgagtc, tgaaagataccgtaat,
tactagcataaagaag, ttgtagctaaagtcta, ggcacagtcggtaagg, ggcgggcgattcgccc, gtctttgcacatcccc,
acaacattgtagtcag, gttaagtgtatagtat, gtgggctgtaacggag, ctcaagtagctgcacc, ttgacttactgaaaag,
gtataaggcccaaatc, tcgataatgctaacgt, accctacggaatacag, ccaggttccttgcgaa, aacatataatctgcgt,
atctatgtggagctag, aagagattgactgagc, atctttaccctacgca, tggatagccttgtgaa, tctctcacattaataa,
caatgtcatgatccca, attctcatcttcgctc, gtcccctccctcgcat, gaagagaaaacgagag, ccagttataccaactg,
atgttatgtatggcat, acaatggccgttgaat, ttttctctcccagcgc, ttctttagcgagtcta, agttgccagctacttg,
acatgctagttcttaa, gtggcgcagtgatgga, taccctgcatgattga, gggatgaggtattacc, actgaggcgggcttat,
acgtacgatcatcctg, tttttctcgtgaaccc, ccttctaccccgaatt, caaactccttctgcta, aagagctacaacagca,
gcaaggatcctaccct, cttctgtactttagca, tgccccccccaaagtt, ctggccaacccggtta, catgtggcaatcaggg,
caagactgactcttgg, aacttaacctcccatc, acctcattatgcaacc, ttttccataggggaac, cgtccctccagggcca,
tcctgtagctacatag, acttataaagatctaa, gtagctgtaggcacta, aaccctccagttactg, ccaggagtgtcacccc,
agtgagtaccctgcat, ggctggcgaattggat, tcatgagaccgaaaat, gtgttccaacgctatt, gcaatcactctaccac,
gtttttatgcaactga, cattaggttgcatccc, agcagccaacgtgtcc, ccattttcgttaataa, ttaggattgggagata,
gcagcacacttttttc, cactgtgatgccaaag, ctgctggaaatgcgga, aggaacgaggggtaaa, gagtattgaaagttac,
gtgggcctttccttaa, aactcagatttaaacc, gttaaaccccgtcgct, tcagggtttgtgcaac, atgattggaaggattg,
ttgcagttagacgagg, gttaacgttcctcctt, tgccacgtttcagtct, ctgcccacatacttga, cctaattgagcattgc,
ggaaccaacaccaatg, gaaagtctatccttgt, tcgtttctgaaatttc, tacattagctggggga, acttccttcctgacgc,
cccttattagtagtat, gatcctgaggcaaggg, agacgacttgtccaca, ctcttgaacatctcgc, tctccgataaaaaata,
atgtctatgagcgctc, catatcctttctacag, ctgtctctggtttgaa, acctcacccctcgggt, ggccgcgcagtggcgg,
cctggatgtgcactta, atagattgaatatcga, cctaattggtcagatg, gataagagggttaggg, agagtaagagttgtgg,
tctgacggctccggag, gtcttttaattggcga, atttctaattatcggt, gagaacatagggatag, agaaaacaaatgcacg,
tgagttacagcgctgc, taagccggtccataat, atgagacgtaaaccac, gctaggatttaagttc, caactataatttgaag,
atggaatagtttcccc, agtgcgttggcgttat, tacctctatttatgtt, aggatttacacgtgtt, gcgaaaggggtttgcc,
acttggttatggggaa, acaacaatggccgttg, tagagaggcataagca, ttgcatgttgttatca, acttatggccaaactg,
gactaggttataatgt, gcttagggaggcagat, ccttacactggcgtgg, tggcctttggctaagg, agtaactcaggcagta,
ctcgtttctgaaattt, tggaaaaaagcctagg, atgtgaccctttaaga, caataacggtgtgaac, tgagtcccccatgaac,
aaaacgtctgcatctg, agagattggtactcac, cagtagtcacctttgt, gcactgtactgtttca, aatcaatgctgcatga,
cactgtaggtgtaagg, atctcatgtactgccc, ctttatgtctatgtgg, cggcaccaaatttcag, tgagatgagcggatta,
tgtttagtagttaaca, acggaagacaaactgt, tttagttcttacccct, tgaaacacgaatttat, caactttgtatggcag,
ccgtgtccccagacta, gtctccttcgtcctgt, tgcatattcacgagca, catataatctgcgtgg, tgcggttttagagaaa,
gcagggggtccaggaa, atcctgaggttacctc, ccatataaggtaactg, taatccaataagccgg, ccctggaggcgtagct,
agcacctctatgcgtc, ataagttctctagggt, gactgaacactaagca, gcactcaacctgtttc, acttattattttgccc,
gccattggtaggaagg, tggggattagagctag, gatagttaaggatatc, ctgtgatgccaaagaa, ggatcacacacagttg,
gagattgactgagcag, ggcgatcactgaaggt, tgcttcggataaaatt, attactcgcctgtctg, agatcaccatcttagt,
ctttcccccactccgg, tattggagtccattga, cagtcccctccctcgc, tctctccacaagaatt, ggtggtttcaatatat,
ggtccacactcacatt, gagcagcagaagcgca, ccctgagcatccagat, aaaaggtgaagtgggt, tataactctatgacgc,
cctagggatcccacaa, cttgagtctgttccaa, cgtcagcttgcaaggc, atccgcctctctttct, ttcgtggacatagtaa,
gttaggtcaggctgca, ataacccctcaaagtc, tgatagcttggaagcc, acgtagagggagcagc, agaggacggaaaggtg,
agctgaatgctgatat, ccgctatagctatgat, gaacgcttctctttta, tactaggtctgatatt, gcacttgcaaccacgc,
ggttgggtcctttggc, aacgaaggtggcttat, ctgagccacgaaaact, gccttgagaatggacg, ctagactgtactgcct,
gttcctctggttggat, cgaattcatgtcattc, tccgcctctctttcta, gcctgcctatattttc, ttgggctgtcctattc,
aacacgattaaaccct, tcctatgctgtacttc, acttagtgtgcttgct, acttaaggatgtgaga, tccataaccacagaaa,
ctcctaaaacagcggt, tggggtagtttcgttg, tctctgtacgtctttc, gattaagaggaagcga, ggctcggtcgcctccc,
atgttcggctcataat, aattgactattatagg, tcccctccgtaattta, tagcattccatgtcac, ccgtttctttctgttg,
aaaccatatctactgc, tgtcggtgcttggcct, agttccatcccgtttt, ctgtagttaaggccct, ccgtcaaacttaacag,
cttaggtattgtacta, caacatccgccgccca, gcttaggtattgtact, tataagaacggaagtt, ttttcacagtgccgag,
acatttgaccctcgac, cccgaaaacattttcc, actgagcctgggtaag, tcgtggcagcaagcca, gaatttttcagcgatg,
acccaaatcatgagcg, aacaatccatctctaa, tttatgatattggcca, tgggtatgcacaggac, ctagagcctgtaggtt,
agtatttcctaagcga, tccttataatatgttc, taatctgttgacctcg, tttttgttggtacgct, ttatgtagcaacgaga,
ctccctatgaactttt, ccaaggcacggcccag, agcttgaagcgtaggg, cgtaagcctatttgtg, ctacaacctactcatc,
gaacctccttcaataa, taatctatcccttcat, ctaccttaggtttatg, gcatcttaaggcatat, tgtctcctttcgtgat,
gagagatagagctgtt, agccccggtgcggtat, tgttggcaatcactta, atgaggggttatcttg, ttaggtcctgattgct,
agcacctaatgtttcc, cataggctaggacgta, agctacttacccctgt, ttcgtaatgacatcta, gcctttattaccctat,
cctacaagcttaagga, acaagtagctgtaggc, atattgtctgaggaac, ttttctcgtgaaccca, ccattaggcaattata,
cctgcctatagaaaga, taatctaatccagggt, ttctctgtacgtcttt, tgttaacgttcctcct, taactctctatgttct,
ggagacaactgttagc, gtgtagtaattaaaga, tattaggctaaaccta, tgtgctcagcttgtac, agaagaaggttccctt,
cattgttcgggcccag, cgggtaacaggtacta, cactagaaggtgccta, agccatgggctttatg, agttggactcctacgt,
cttaggctagaggatc, aggtagcagtatctat, ttgtccagacaaaaca, atcagcatgcctttct, aagacgaaatgaattc,
agacacaggcgcctgg, ataaacgcttgaacct, aggctagattgcatga, cacagaggtaatcccc, ccccaaatgtagacct,
aagaggtgccctagct, aaagatctcgcttcaa, ttaactctctatgttc, gaggtctagcaaagag, taatgggggggtaggt,
cagaaaatttaagatc, ctagatccagaataga, gcagcgtaagcctatt, cgtgggatgttgagat, gagtttttagagacta,
aggctcaggctggtat, caaggcacggcccagg, aacgttgaagacattg, aatgttcggctcataa, tacctgagacatgtta,
ttgcccatcattacag, attgtgtccccgcact, gctaggctctgcctta, tcaatgtgctgagtta, gctggcaatgtcgaga,
agtgacccattaggag, catatccccccccaga, tcatgctcataaggaa, caatcttcggttatta, caagcgaattgcttta,
tagctgtcatattata, gatcctcgcaccttgt, gggatagtgaggctat, atttctcaacgaattg, aacatcctggtaatta,
caagtgtcagtgcctc, tgctccttacatatgg, ggtgggcgatcactga, gttaccacccatttca, ctaggaaggaaaacgt,
ggtctataagttttgt, tagagcattggtgtta, gtagatgaggaagacg, caggggttgtgccagt, ttggggcaggcggcag,
ttgttaaacgttagat, tcagtaccagaccttc, tgaccggccgccccta, agatctactgttctgt, acacagttgggcagca,
agggaaatacccttct, gcttatgtataattct, gcgtaagcctatttgt, acacctggtgaatgtg, aaatctttgttcagcc,
gccttaacacatggtt, cctgcaagaatactta, ataagtgtggttgtct, tccgagagagaaatct, tctgttagtttggtac,
aggtctggtggggcct, agagcagcacctgttt, agatgaccttagtgtc, cgtgcactggaatccc, tccttgcgggggggga,
aagtggcgcagtgatg, cagattcagtttaggt, tgttcggctcataatc, taacgttttaggtaaa, gcgcctccagttctaa,
gaccgagagaggtgga, tggccgttgaatccca, gctagtcacatgggtt, caataacgtttgcacc, gtataggaagacctta,
caaataagcaatgcat, ctttgggagggtatta, ggatcccacaatgtag, ccttataggctgtgtc, gagagaaatgcgaggc,
ttatatggaactcctt, taatctgcgtggagca, gacacaaactcggggg, gccataggtggcaaca, tcttatttgttcctag,
tgcaatcactctacca, tccagttgtttggtga, tgatgcactatattgt, ctgcagatgcaggtac, atgtactacttatatc,
ttggcattttgacgca, tgaacttattgtgtga, agcaggcgctattctg, tgagcgctagagaaaa, gggcccactgtaagaa,
attaccgtcaaactta, cactgatttgagctcc, ccattgaaaagtatta, aaacatcagctacgaa, gcaacttatgtaatac,
tatgtcccttgccctt, tgtggccctgacatta, ttggtgtatcttttgc, tgcaaacgtacgatca, tgtctctggtttgaag,
tttgtactcacacgat, attggagacctggggg, tcccttattagtagta, tatagtaccataatca, gtgtctcctttcgtga,
ggaaggcacatgtaag, agggcccactgtaaga, ttgcccctctccagaa, ttctatagctacctgt, tcttattctgagttag,
agctgtgagagtgttt, ttagttgtagtttgat, tgtaaagtgattgctg, ctatgtgttgctgggc, ctttgaactctcacgt,
tcccgaattcatgtca, cttcttgagtgcttag, gtcattaagctgacta, cctcggtgcctggata, tataattaagctgacg,
ctaggacgtaatgtct, atacttataggcattc, aggtctgacggctccg, tgaggccaggtttatt, gcaagcgaattgcttt,
aaattacgcagtttta, gttgaataaacaccgt, gctcttccttatcatg, cttgggtctttatgct, agctttttcatacgat,
gccagatggactgggt, aaattaccgtcaaact, gggtaagtaattcagt, gtagtaacccctctga, tgggatgtctatgagc,
ttacgtggccaaaaac, agcaaagttagggtcc, tgtggaagtacaggag, tgagctgcgaccctgg, atatccgcctctcttt,
tagttaaacatcattg, gtacacttacacaacc, ttgaaaaggatgctca, gacattgtgggtaaat, gatgggcactgttgca,
ctgtatggctttgcaa, aggacgtaatgtctaa, tctttgaactctcacg, agggtttgtgcaacca, tgtgagacacggagtg,
ggttacagaataagcc, tccatgcctttgggag, catgctccaagcctag, taatttagcatccgag, ctgtttccgttataaa,
attgttgtgtacacct, acgaagcatagaattt, cgttgttgacagctta, gtaggcaaattagtat, ggcactgtgcaatact,
ggattagagctaggct, aacggaaagaaggcag, gccctgaaaaacgcag, aaggtctgacggctcc, gtattaagttgtggaa,
gcgcctctctttctgt, aaagcccttaaattag, caattacataaggtgt, taggtttcttgtctgg, ctgcatattcacgagc,
tagggcttactgggct, cccccacttcgtcacg, cagtaccccttggaga, atcgataaatcttact, gctggtcgcgctggtg,
acgttttgctcaaaat, accgagtgtttacatt, tgggcacctgagttat, tcgggtcaataggctg, cgcctggggactctgc,
tccaacgattccactc, ctgaacaatccttctc, aaccttatgtagcaac, ttccacaaggtgctga, catttgaccctcgacg,
tacttatctctcctgt, cttttggccttactca, tagcaggccacgttat, cttagggacatgagtt, tattgtctgaggaact,
tatgtagcaacgagag, ttccatgtgtcataac, cgtatggcataaatat, ttcctgtgtcaccgac, ttatgtaagagcgaat,
cacgtggcaccagatg, tgaatttttcagcgat, ctgtataaaggagacc, ttagacctttgattac, cctgaaatgacccacc,
gatttccgtttcaact, ataaatggtacctaac, ggcaaaaaaagctcct, catgcctaccttggtc, ttaggctagaggatca,
cctcttccaatactta, atgctggatcaggtca, aggtggcttatgccct, ttgctggcaatgtcga, tgttaactactgttta,
ctgatttgtttcaagc, agcgtcattttttttg, aaacgtctttggtgct, gctaagcttcctccat, attgcaatctacacct,
cttttagttgtaacgt, ggccgtagagggcagg, acgatcattgttctca, ggagggacgtggctgg, gttcctcttctgatag,
tagtctgtaatacttg, gccttaaagaagaacg, aacgtcagcttgcaag, gtccattgagagctgg, gagttgcttaagacaa,
cttggtcgtttctgct, ccaccgtgtccccaga, ctgtgggctgtaacgg, gacttactcttattta, tctaaaaagtggttca,
gtccagttgtttggtg, tactttgtagaaacgg, gcgctattctgagccc, cttaacaactcctgag, tcgttataaaagcaaa,
tgcattgagctgtgtc, ctctcgcatccttgaa, attgtcatctttcgtt, agcacacgtgagcctc, tacatcatattgtagg,
cgggatggtctgaatc, agactcaaagtttggc, ggatgcttttgttata, gtagatctctgtggaa, ctcacacacttaatag,
agtcataagagtctgc, tagcttaggtattgta, ggtgccttagggacaa, attcagaccgggtctc, aaagcgaaaccaccat,
caccgttgtaatccca, ataagtgtttgaaacc, gtttcacctttttgga, gacaattgcatgcaag, ctttacccttacgtaa,
ggtgagggtctgtata, tgaaagcccaccctgt, tgagtttgcttaagtc, cacacagagtaggaat, gcataaggcagttaaa,
gtaacggagacttaca, ccatacctatgtcctt, ggcatatcacgaggta, acccaggacgtgatgg, accatctgtcattctg,
ctggttataataatcc, gttctcctttcaggag, tatggagggctaaaga, gtctcgatcctcctgt, ggggtccatttcaagt,
cccgcaaagaattctt, tgtcttttttacaagt, aacgtctggttttcct, acggtttctaatagtt, tctgtataagatgtgc,
ctcctgggcacagtcg, acgacaggtgttactg, tgtgtattctatccag, tcggtgcctggataag, gttagactctgtatta,
gcagacaatctgaaac, atacctcactgaaatg, caatttataggcagac, ggtgttcgttaggttc, ctgcttagcgtcccaa,
gcaaacgaaagcaatg, taaactaccttaagtt, tcccaattaagttcaa, gagggagttctccctg, ttagtgccctgttttc,
ttaccgtcaaacttaa, taggctaggacgtaat, atcgtggcagcaagcc, ataatttgtatgttcg, caccgtgtccccagac,
ccttctttttcgttta, acagtcggtaaggttc, cacagcaccactcctc, cattaggcattagtgg, gaacaagagagcctgc,
gagcattcttctctta, cctgcccatattgggc, attcaattattatctc, gtagtcagttcttgtt, aatgatcaaaacgatg,
agactgattcactatt, gcaagtagggtaagag, attaatccaccatcta, ggttaagtttatgttc, ttagagaacagagacg,
tacaggcgttagacat, ctcacctaaagatttg, tttccagggtattagt, ccacatgaatggtgct, caggcacctcagaacg,
cgtgtaaacatactgt, gatcccagagaggggt, ggaggattggtcctgt, accgtgtccccagact, tgctcttaaaggtatt,
cctcctggattgaaac, aggaaagtggcgcagt, tttagggggggagctt, cagttgtcctacattc, agcaatacaacgaaaa,
gaacacttacaggttc, gtatttgaactactag, tcttctgaacagcccc, ttctcaagcgaatgaa, tgacgggtgcatgtaa,
gatggtgttagccact, gcagtaaagcagctgg, ggcctgtaagttttta, tgctattactcaacct, acaaaaacgctatgat,
gtcactcttccaacac, tcatgggaatgagaat, ccatcccaaacgtgaa, acttagagtagggagg, cagaacgtttgcctat,
agtgggttagagatat, ggctcaccgttgtaat, gacaggggttaagatg, aactgcttccttaagg, atattaggttagatgt,
ataaattgcccactga, tgcagctaaccttgga, ttactttgtagaaacg, ctcaggcgggcgattc, cgaactcgtggactga,
attatgtattcgaagc, tcctggttaatcaacc, gtgcaaccaagttggg, ttgacgagctgagata, agaattgttagggcag,
gtcaagcgttgtgttc, atgattgacatatcaa, gggagtagggagcgct, gggaaattaatacggg, ccaacaggccaatgct,
ccttcttggaggtgtg, cgggcccaggttggcg, agctaaactgtctaca, actgtaggcttgagag, gttgtcaaggtgaatt,
taactgaatttgaagt, aagtgtccatctacta, acaatcctgtagctac, gcaaagttagggtcct, gtaataaaaagcatcg,
tatggaacttagtaca, atcaaattaccgtcaa, ttaggcatttatactc, ttaaactagcagcctt, catccatagcaatgtt,
ggatcatttctgtgca, tccatgtgtcataact, tactgctcccatttac, tcaatggagggggtgc, gcttaggagacttcac,
atcttcccttatcata, aggcgggcgattcgcc, ggcactagaccaatag, gtgtctacgacagaat, tgcccaaacacatttt,
ggcactggatgcattg, aaaatttataaggctc, acagatcactcttggg, acatataagagaatgg, atacattgagttacag,
gttaaacctctgtgca, taattttatgcgagaa, cattctttgaagtatg, gtgcaaacgtacgatc, atggccgttgaatccc,
cattccctgtttagtg, caggggtaaggcatca, agcacttatggagttg, catgctttaaagacac, actaccacacagagct,
cgatttttgcaaaact, tggatataaagctctc, caataagccggtccat, ctgagatgagcggatt, ttggggtagtttcgtt,
ggggatttgacagaga, agcgagtctaccattg, attgacaaatggaccc, ttcacacatgaggtag, agattagatcttacta,
tgttacctcaccaaaa, ttatcccatgagttca, caccctccgagggcca, tgacaacaatggccgt, ccttgtctttacccct,
accctaatttagcatc, atttgaccctcgacgc, aaagtcacctgatccc, gactattatagggact, ttgatgtttatccggg,
atgaaaaccggtagag, atgccgtgcttcttat, atatacgaataaattg, acagtcttactgcatc, tattagctgctgggat,
taaaggagtaggccat, acttgggcggcttagg, gtgcatatgacctagt, acagcatgagggatgg, gacataggctaggacg,
ggtggggggggagtat, caacaatggccgttga, aacctctatgcacttt, ggtttgttgttgcaga, acctaattggtcagat,
gcactccagcgttgac, tttcgtacaaaactaa, cccaggagtgtagtta, gggatgccaaaaattg, taagctcttaagtgct,
gtgctgcatgtaatct, ccgcctgctgaccatg, ctgtgtcacccagccc, tgtactctaggtaaag, cacatccggtgccctg,
cgatcaaaaaaaaggg, gagttaggtagcagta, tgaactctcacgtcta, gcacattcgtctcaag, gtgagaaaggaaaacg,
cctgggcacagtcggt, ttgtgggtaaatttca, ttacatcactcattcc, gagagtccttttagct, ttcccccctgcaaagt,
ttctagtaaaatgggc, cacgattaaaccctgt, agttgaaagggatatt, ttcctaaacaccacac, tgtgcgcagtaaattt,
ggtcacagtgtaaggt, atagagaaaggtcgga, caaaagacactgatgg, attccgtttgaaaaac, gatcccacaatgtagg,
tgtgcgcccatttctg, ctctgtacgtctttca, taggacgtaatgtcta, gtggctcaccgttgta, cttgcccatcattaca,
atatctaagcttaaga, gtgcgttggcgttatc, cacagtcggtaaggtt, gtagtattgtcttgat, gccagggggcaaaaac,
ctttaaccctgagaca, agggggcctcattatt, attgtcctctcagtat, gattggatattcttcc, aatattttcttgaccg,
gaagtggttccatggc, cccacgtggggacggg, ccccgttggggaggga, tttttatcgtaagaat, ctctcccgactcttag,
tgagcaccacactgtt, tgttcccttccagatc, aacctatatccgcctc, cttcacgactctgaaa, aaggtttttgggctcc,
ctttagcgagtctacc, tatagctagtgccatg, tggcagaattctatgt, gtccctggccggtgtg, acctgggaatgttgct,
tatcaggtctgtgctt, aaaaacgtggggggac, ctgccactgttggtac, acatggcatagttatg, ccctctcatgcttcat,
atttcagtaccccttg, cgtttaaaaaatgaag, aaactaagtggtcatt, acggagacttacaggg, gccaataactgtagaa,
gtgagtaccctgcatg, taactgacattgtggg, gtgtccagcctttatg, agaacatagggatagg, gggcagaaacccactt,
acttatagtcgtctgt, tgagtaccctgcatga, tttctcaacgaattga, gtaggggtaggggctc, gttgtgaaagagtgtg,
ctttcatggtagggtt, gtctgggaccacgttg, ttcgggcccaggttgg, gaagcagacgccagag, atggttcccatgttgc,
gcctgccatcttatag, gtgcaaacatcaaagg, aatgcgtaatacaatg, gactctggacacctcc, cccttgatttgctttg,
accaaattaggatatc, ttatggttccaggggc, ctcgtgccaccactat, ccgttgaatcccattg, ccttaaagaagaacga,
cccttaatacacaact, agcgctagagaaaatt, ttagttttagaaggca, gctcaccgttgtaatc, atccttaggctctcaa,
ggacctcacaatgggt, actttttcttttggac, aaacatataatctgcg, agcgtaagcctatttg, ctagaaccgcaccccg,
cagtgtcccatatgat, cttgttgtctcaactc, atatcacttcacacaa, tctatgtgttgctggg, atagattccttacctg,
ggtttgaatctcttga, agtaacctgttaaact, ccaagtatgtttgtta, gaccctaatttagcat, caagcaccttttttaa,
aatcgtagattaaatg, tataggactaactgaa, tagacaggagagtaat, tctccatcacgactat, aggtcttactgctata,
tctctaccgggtttcg, tattgtgtaacaagca, atgcagctcttttgtg, agttggcgtgtgttac, aaagacctttccactc,
gttaggtagcagtatc, ttaagggtggggccat, acgattgaagatgaga, gtattggagtccattg, ttcaggcggccagtcc,
cccaaaacttgctaga, ggttttgcaactataa, gtctgtaaggaaatag, gagtgaggtgactgta, aggtttattacacacg,
ctgaccttatccataa, tgagctatgagctcag, atctaaacttagtcta, gaccgataaggcacag, gtgggcgatcactgaa,
agtattggttctgctg, tgaggaatctcgtttt, ggcgcagtgatggagt, gagaggagtggtagct, gatagagtctcccaaa,
gcctccccaaatacca, ataatgatcctctgtg, ttatctgacgggtttc, tgctgacccttagcct, accgtggacactgtgt,
gagatgatttgcttag, acctgttatcagtaaa, cccacgtggttaaacc, ccctagtatctgacaa, ccattggttaaactct,
gactacagtggtgggt, gagtgcgttggcgtta, gtccccttaggagccc, gttagaatgcggtcct, acaacctaccaacccc,
ctggaattcctgccta, tatgtcattggaagtg, tatcgtctcatattct, tggttaaaccccgtcg, gtagagcttcttcagc,
tgtactcacacgattg, tctgaatgcacagtta, atgatgctgtacatcc, tatctagaggtaaaag, aacagctacttgtgtg,
aagatataggactaca, tgctagtcacatgggt, ctattgaatgttattc, tgtgtgtcgagttccc, gtgtttcgtaattttc,
ggacgtaatgtctaaa, cgggcgattcgcccgc, aaccttgttgaatgat, gcacagcccaactcag, agctatgtccccttag,
tgcgcctccagttcta, tgcttaaggatgttaa, tccagggaaccgggca, tcttttagttgtaacg, acgtaatgtctaaaac,
tgcgcctctctttctg, acgtggccaaaaacat, acgattaaaccctgtc, ggaagcttagctacat, ctgaggcgggcttatg,
gtgaagtaataggcac, atatctgctaggaatt, tggtctgaatttcagt, actgttgtcatcaacc, ttagcacgctcagctt,
cctgaattaccagggg, atgttgcagcatttag, agttacaatgctatac, aacctcacctttagtg, aagcttattgtgaaag,
cacaggcgcctgggga, tgattacccctgacat, gagggttaggacagaa, taataggttagtgaca, tatctaacgtaggaag,
tagagccaggcagtta, cccttctaccccgaat, caaaaacgctatgata, agtctatatagtgtaa, ccgggatggtctgaat,
gtaggggccactggtc, aatgcctttttcagtc, gcttcacgactctgaa, tattgaaagttaccac, attagagctaggctct,
aagtgacccattagga, tgaccctcgacgctgg, gctttagaccatgaca, ctaggctttttttatg, caccacctaatacctt,
gttcccttccagatcc, cagtgaatggcgattc, aggttggcggggaagc, actgccactgttggta, tgcgtagagtgtgtac,
agccggtccataatta, ctacccgccagctccc, gtttgaaaaaaagcga, cctattaatgttgttg, atataccatgcccaga,
agttttacagattccg, tggcccacgtggttaa, gtgaactttctccaag, acaatatgatgtatca, agggacattaagatca,
tcgccagtggtgtttg, gtctcagcgctcctca, tccaataagccggtcc, gtggtgaccattcgtt, cagtgcggggacagac,
ctactgttgataggag, ccccttatcttaggtc, tgcaacattccacatc, tccctataaatctatg, cctcccgccttagtgg,
taggtccattatttgg, gaggtagatcatacaa, gctgaaacgatctgcc, tattctctgtacgtct, ctgaagcttatctata,
tcagattaagaaggcc, aagagattcttatgac, tcatatcccccatgtt, aagttggactcctacg, ttaacctggccattta,
aaaaccggtagagacg, attatgtacagtgtcc, cgatattggacttaac, tatgtgcgcgtgtgtc, tagcgagtctaccatt,
attataggtcagttaa, aagttattcttgtgtc, acgttgctcagtaact, gggtaaattgattgaa, ggtaaaattgattcca,
gttaggcatgttgctt, tggaaaacgtaacagt, gtagatgtgcaaacgt, gatcttaagtgtaccc, gcgttttgttaatttc,
gcaattcctaagcagt, aagcgttagccacagc, attatggagcataatt, gctatatcatgtgtgc, aaatactcatggtata,
atcctaagcccgcccc, atgatggacacaaacc, ttgggaggtcaataga, taaactagcagccttc, aacgtctgcaattaca,
actgctgggaataaga, tcactcttccaacact, accgttgtaatcccag, gatgaatggacctcaa, ttaacaactgtctctg,
tctgtacatgctgtgg, gtaagctgcatagttt, tatgtctatgtggacc, tttcatggtagggttt, tgagggtctgtatatt,
ttgatagtagagtggt, tcctgggcacagtcgg, cataaatttttgggtt, gtgagggtctgtatat, aaccctgctcagatat,
ctttagtatagtgtga, tttgcaaaacttcccg, cagttcatctcatttg, ccggaggctgagaggg, ggacacagccaaattt,
ctaaccttatgtagca, ctggcccacgtggtta, gtagcttttgaaacac, ttaagcttccagagct, aatgcccataaaccct,
acaggcaagtctgtgt, gcagtcctcctcagta, ccagggagctcaactt, actgtctgccattaat, ggacactgttaagcaa,
tgtctagctttccagt, agaagtaggttctggg, cccagcgattctgtta, tttaacgcacatttat, ctgccttaaggcccct,
ttatctcggctcgcta, tataaaagttctcctc, aaccagttctgaagtc, gggatcccacaatgta, taagcagacttgtacc,
ctgtaatttaatatcg, tgcatgttccaccatt, caaattaccgtcaaac, ctagagggcgacatta, gttatgtctaaatatc,
gatagataagttacgg, gtgtcgatgagcgcta, agaggggattactgca, aaaatactcacccttg, tgtcggactgtctgac,
accgtcaaacttaaca, ttagaaacttttgaac, aaccatgctgtgctgg, tgcttgcgcacctcaa, atcataatgcgattaa,
cacaaatcactccccc, aaccgccttggtttcc, ggtacattactgtaaa, tgtataccactctctt, cctggttaatcaacct,
aatatgtgttgtaaga, aggagtataagggttg, tcccctaaccttatgt, ctcatcctaggataaa, acttggggaggcatca,
cttagacccagctttc, tttagtgtaagttcat, accctggaggcgtagc, ctacctagtttaaatt, ccttagctgtacttct,
ccgctttcacgccatt, atgcctcttagctctc, gcccgggcaaagactc, cctaatttagcatccg, aagggtgagatcagat,
aatgattacccctgac, gacctttgattaccag, cctaaaacagcggtct, tttgaccctcgacgct, tgcaattgtactgagt,
gtgagacacggagtga, acgtatggcataaata, ataggtattttagagt, gtataaaggagaccca, gattctagtactaggt,
gcagaggtgtagagta, agcctgatcccacctc, ttgtctaatctcatac, atacattctgacacct, acagcaatcataaagt,
ggaaattaatacgggc, gcgtgcactggaatcc, tcactattattcggtg, taaggaactctcgcat, gttggggagtccacag,
acgtttaggcttttgt, cgttttgctcaaaata, ggtcttctggagtgag, taaccgaaggaataat, gagacggctcctagaa,
tcctctctcttttcgt, gcatcatcttgtgtgc, catgctgtgtagagac, gcaaacttcatgacca, tccatcccaaacgtga,
tctacttaggctgaga, ctcttcacataattgg, atttgaaaagattcga, gcacagggattggtac, aaccatgaaatgatga,
ccctactgggaagtta, gtgctcttacgtatac, ctagtagtgaagtctt, ggatgctggttccttc, acatcgtaaggaggac,
catatgtgtatgatct, ggatagaccaaaggtg, taatcatgacagggga, tttaagcatctaacgt, attgttcgggcccagg,
cttactattagttacc, atagattatttggagg, acacgattaaaccctg, cattaacggacagaga, ctaagtctgaacaaat,
ccaaacttctcactga, ctgaccgttgtgaaaa, ggatagccttgtgaac, gctttccattaggtta, cctgccttaaggcccc,
aggtgagggtctgtat, gccgttgaatcccatt, tatagattgaatatcg, tacgtagggataaact, tgacattggggtaggt,
acacccttttaagttt, gaagtggccagaataa, ccaggcccgcagagct, cttaatgctccaccac, gtgagttgcataatga,
cccccagtttcacggg, aaaaatatcccagact, aggctgttgaagctta, taaacgggagaaaaga, ccacttacaagggatt,
ccagatctggcgagac, ccggttctggccctgt, gccccgttggggaggg, caaattctttaggtac, ccctctcttatttatc,
aagaaccccccccctt, cattagctgggggagt, tagaccgtatatttga, cgatgagcgctagaga, tatactgggctaaatt,
tacccctctgctactt, tcttctctaccgggtt, tccggtttctgcccat, ataagccggtccataa, aaacttgacaacgaac,
ctctttcaggggttat, gagaatcctaagcccg, gtgcaatgagtgtaag, agctgggggagtcatt, gcagctggctaaaact,
ccttagcctggaggca, caggatgatatcagtt, taaccaaattacccat, tctcaagcgaatgaaa, tgcagctgcgcgacct,
attctctgtacgtctt, agtgtcgatgagcgct, agtgaaaagtatgagc, agtatcttctctaccg, aataacggtgtgaact,
cagatgtaattcacac, gaaaaccggtagagac, ccctttacataacctt, caactagctgatattt, gatgtttttcgtttta,
ttctcaacgaattgaa, cagccaaactgtttgt, acaggcgcctggggac, catagtgctgggccaa, atggtgttagccactg,
agtctcacagattaag, actaagcagacttgta, tgtgtctacgacagaa, tctgtactccacagtt, tgcaacttatgtaata,
atggggtccctagggg, aggaactctcgcatcc, tttgccatgttaaggt, ggctcataatctgtac, gcctcccgctttcacg,
gcttgcagtgaggtta, aataaagaaccatggt, aagctatcgaatattt, tgattaggaattttgc, gaaggcacatgtaagg,
ctgccaaacagagggg, ggcaaccttcttagtg, atagaatggctgatca, ttagctttttcatacg, cccctttttcatggga,
attgctcctagagagt, cagatggcgctgcttg, cagtgctcttacgtat, ggcttaggcagtggaa, acccgaaaacattttc,
taaaggaacgaggggt, gattggacaaagaatc, tgtgcaaacgtacgat, caggtcttactgctat, aacgacattcatttaa,
tcaccgttgtaatccc, aatcagccatgtaact, agttaggggactaggt, tttgtatgtaatccaa, tgaagaacttaccgtt,
tgcggtgattaagcaa, taccctctatatgtta, gtagagcattggtgtt, agttagactctgtatt, tctgggcaccaggcta,
cactgttagggaagga, ggttaaaccctgtcac, tgactaggtagaggtc, gttagacccagtgcta, gcgcaggggatggggt,
ccggtccataattatc, aataagccggtccata, ggtgcaggactaggtg, tgcagtgtgacaacgt, aactcctatcataagt,
ttcctctctcttttcg, gtctccatgaattaag, gcgattttaaaatgtc, ttagactgtaaactgg, ccgaagaatctttgct,
accctggagacccccg, tcagtctaccagactg, ccgaattagccaggac, ggcacttgcaaccacg, cctcctgaagcttatc,
tgttgaataaacaccg, aagaagttatggctta, aactgggccatgtgag, tagagctaggctctgc, tgtaaagcctctgaag,
ttcagagtatagtaac, gctacacatatttagt, tcgtctacccgccagc, catcttaggacttgac, atttttaatattgcgg,
atcgtaagcactgagc, gtggcaccagatggcg, ggatctgtcaatgaga, gacttcctgaggcttt, tatttatgcataaacg,
acatcatgatccctga, cctcgctgactctggg, tttggcgtgattattt, gcggtggagagagacc, gggcatccccccccaa,
ggatagtgaggctatc, cttagccttagattag, tgcccataccttaata, tttggggtagtttcgt, gggtagttccgcccat,
cgggcagcaatacttc, aagtctagccttgtgc, tggatcccgacagctg, aatcctaagcccgccc, tgccactgttggtacc,
tcacatcaatacgtaa, atgattacccctgaca, ggcatccccccccaaa, caaaagcctgtggtgt, ttctccgttttcacac,
agcttgttgacaccac, ttcttatacagtagtc, ggaaaaccgaaattta, gagtgtgcctagtttc, tcctacaccttttatc,
gggctccaagagctcg, tgcagttagacgaggt, actatagtgaggtgaa, gggttaatctacaaag, tgacacttatgcttga,
ctactgggaagttagg, gatgaagctcagcatc, acctcagaacgtttgc, tagaggggattactgc, ccggggtgcctccggc,
ccatccaaatgcacat, ctacagtcccctaaac, tctctcttaatggctt, cttgaaggatagtaga, gattacctggatggag,
tcgtatgataattgta, tgctgatttaaaatgg, tgagacacggagtgaa, tcctgaagcttatcta, ccctcggtgcctggat,
tgtagattacttaagt, aacttggtctcattga, atgtcgtacattctat, tttgtgcaccctcgca, acttagtttcaggtag,
agtacatgacctagtg, tgcataaaccttaaca, attgtgatgtgggtac, gccaagattaataaac, ccgtgttagtatactt,
gggtatggatttaaac, agtagaaggggctaaa, gaaaacgtcctctaaa, caattttgcgcagtca, tctgttattcactgca,
ctaactattttaatct, gggtggcttaggtgga, aatggccgttgaatcc, ttttagggggggagct, ctctgaatcgagagat,
ttggtgcaggaaagtt, cataggagggaatgct, cataatagactgtagg, attgttctgggcacta, aaatatcttgtcttgc,
caatcttattgcagct, aaggaattgagccagt, actctcgcatccttga, gtgtgctcacaaacta, cctgtaagttggattt,
ggtcgactgtagtccc, ggtttaaggcactggt, tgtttgtactcacacg, catctgcaacctaacc, tgtctgaactgtggga,
acctggtgctctttgt, tcaaaaggtctgacgg, cggaacttttgttttt, tggtagatcttcgatc, gttgcaaagggctgca,
cctagccagcccgagc, cagtcggtaaggttca, catctaaagagtgaaa, cacaggggatatgctg, ttgaataaacaccgtt,
tagcctgttagaccct, gcagttagacgaggtc, tatgtacacttactca, ttttagagtacaaacc, cgtgtccccagactag,
tgtgcagttaacgcac, gtataactgtgctgaa, tggggcataaagaagc, cgagtttatgattttt, gcgcaatccctcctct,
ttttgcggggggggga, aaaggggatttacaga, cggggtgcctccggcc, tgcatgtaatctgctg, ctttgttcaggcccgg,
cgccttgcggtctcca, gagttagccaccgttc, tttagacccttgtctc, tgaggtagaacactga, tgtgaactccaagcta,
gcgggcttatgcctat, tactgggctgttcaaa, ccaatctcagcataat, taacggagacttacag, gatgtcaaactcttga,
gtaggcactgcaaatg, tgggcggcttaggcag, gttaaggggaaggtag, gtgtaagaacattctc, actaagaaacaccttt,
cccgcctacctccagt, tataatggttgtttgt, tacgtggccaaaaaca, gctagatccagaatag, cccgctttcacgccat,
cttagcacgctcagct, acttcttcttgtcacg, aaagctatcgaatatt, aacatttgctttgccc, gtatgagcacaaatga,
tagggatcccacaatg, caagtaactaagttac, aaattgctaacgttta, catttggaccagggag, ggcttctggtcatagc,
cagaacctatatccgc, ggtagatcttcgatcc, tcgtatcccctagagt, caaggggcgttcacac, tatcttctctaccggg,
tcccttggtctaacag, tatgtttttgggatgg, catatgcccacctgta, gacttatctcccccaa, accacaaccctatctc,
tcttaccctacccccg, atgtccggggttcaag, ctattcagtgaagctg, aattctgtctatacta, gcgtcattttttttgt,
catccgccgcccaggt, gtcccagcgattctgt, ggatgccaataacacc, gaacctggagaacgtg, ctggccaagaaggtta,
aagagactgggtagtc, gcagtaaatttactat, ttggggctcaggtatg, ccatacctatagatac, tgcagagctgtaattg,
atttagcatccgagtg, ctgggggcaggttaca, aatccaataagccggt, actgtgcatcccactg, gacttctacctttcta,
ggcgggcttatgccta, tgacattgtgggtaaa, gaggttagtatcacac, accttatgtagcaacg, tgcgcgtgtgtcttta,
taaaagtaagccccag, atgtttgaagcaacag, ttcagcggaagacagc, tccctgagttattatg, cacacccttttaagtt,
ggaactctcgcatcct, tgagcaaaaagtcctc, aggtcccttggtctaa, caaggctccttactgc, cataaacgtcagcttg,
attaagagctataaag, cttggacattaatata, ttgggcttctgctgcg, attagccaagtcaaac, ctaagtttccaaatgg,
ttaggtattgtactag, tgagaggcacaccctc, aaagcctagtgtgttg, cgaacctccccactgc, tctgacttcgttagca,
cttttgccctttcgca, tctgattcggatcaca, actgttagacagacct, gtgtattctgccaact, gccaaaaggttgcaac,
ctcactactgggaaga, caagaaaaggatgcga, tgtagttaaggccctg, ccaagaggtcagttag, gtagatcttcgatccc,
cggccagcctccccgc, tcaagacatgctttgc, gggtccagactgtctg, atactgtcaagtggca, taaacctctatgcact,
tgagtttagtcctgat, cttgtaaatactgcct, tcacccttgacctcag, cagcacctctatgcgt, ttagggggggagctta,
tcttgggtctttatgc, tctccctatgaacttt, cctagaacacttacag, cgattaaaccctgtct, gcttaggcagtggaat,
ggtgcaacagctaagc, agtgggccaagagtgt, acggaatgttagcaaa, gcaacaactcctctcc, atgcaatattcctgtt,
aagcaagtggtggaac, cgcggggtcggtgagg, ctctatgcattgcttc, atccggaccctaattt, gcagcggtgccatgtt,
tgcaaactgaccctga, attgattaagggagga, acttttcactaaggac, aagatggagtgttcaa, gctctcgcatggtgca,
atagcaaaattcgtat, attataggactaactg, attaggcattagtgga, tcaacattttttccgt, aaagaagggcttatcc,
agtaaacctttcctag, ataggctaggacgtaa, ttggctacttaacatg, tgcctatgagcctgcc, atgccataagcataag,
tagaacattcgtaaga, ttcgggaaaaaaaact, aacagtttcacattga, ggagtcagcatagcct, ctagtttttgcatggg,
ctgaaatgacccaccc, attgtcacaactctag, cccttttgtgtatccc, ggaacgaggggtaaaa, cagctactaagaagtc,
ggagcttagtttcaaa, ttacaggtaacctttc, gaacagaaataacgcc, agattctctgagaggg, gagttttggctgcagc,
ggattggtgggacaaa, ccttacatagaatagt, tccccataaacgttta, cttcactcaacacaac, ttgggatggcctttgc,
aaaaacgaaggagtta, tttgctggcaatgtcg, ctccccgcccgggagg, tcgctttttttatagt, aggccatgtctattta,
tgggctgtaacggaga, ttccaaagctgctagg, cccacccttgatttca, tagtagacaaagaccc, gctgatgttaagtggt,
tcaagtagctgcacca, ccaccggaacagaaaa, cgaaaacataacttct, acagaaatgcttaggc, ttatggcacacacccg,
cttatgtataattcta, gatagatacaggacac, gaaaatggcaaggtga, tacggggtacattaga, ggattttgccattgtc,
catagaggcttaagta, cttcttatttttaacg, gtcttctcggtgtcct, agtgacagttagaccc, gctaggacgtaatgtc,
tcaaattaccgtcaaa, cccatacccttttgcc, ttaagggtcctagatt, tcgacaatttagaaga, atctgaaagatagatc,
gcagcagaggctcatc, tatcaatggagggggt, tttggttaggtaaaca, aatcttcggttattac, cccgcctgaagcccct,
cttgatgggatagcac, aaaatatacaggtcga, cccgttggggagggag, agaaaacctcgaaact, cagataaaccttagca,
cctggagtagaacgtg, acctttagtgtgcagg, tgagcaggggaaatcg, ggctagaaagtattca, aagtattattgatgac,
cttctctaccgggttt, cttactgcggtctcga, tattcaccctaacata, ctcttccttatcatga, ctctctactgattgaa,
gcaactgcactctgcg, tcccagcgattctgtt, atcaacggaatataac, ttggtagatcttcgat, cctggactgaattcct,
taggtagcagtatcta, caagaaacgttcaaag, tctcatcctaggataa, actcgatttttgcaaa, accttagccagctgat,
tgtgatactgcatcca, tccataggggaagcca, atgctgtgtagagaca, gacaaatggaccctaa, cctacaacatgtcagt,
accttaaatgatctat, ggcagtcattacttct, gaaggctgacgcaggg, agaacctatatccgcc, cgcactccctctcatc,
aggaagtgaaggtgta, agttatgtcccatctt, agggttagaccctgtt, ggttaatttacacagg, ataaccgaaggaataa,
tcaattttgtcgtcta, tagaggaaagtggcgc, cattttctttagcgag, ggaccccaactctatc, gcaatcgcggctcatg,
gcatgctgtgtagaga, tttctggaaaagcgat, aaaggaacgaggggta, gtgcatgctccctcgg, aaggtgcaggactgcc,
gccatgggctttatgc, atcgtatgataattgt, ccaaggttctgagcga, tagataggacacacag, ccagcctccccgtctt,
tgacattggggaagtc, tgatttgtcacttcaa, gttgatgttgggtgaa, atacaagcgaagtgtt, ataacactccctatac,
ggaaagtgaaatcgag, cgtagttcatcttccc, cttctatagctacctg, cgtatggcctagactg, ttggcaattttggatc,
cagggttcgggaaaaa, tcacaataagctagga, tcagtgtgactctacc, gagattgtgccatcga, tacctcatctcttcag,
ccctccccctaagtgg, acaagaaaaggatgcg, ctttgtacgtgtaaac, gtttgtactcacacga, acctcccaaagcttgg,
tttcgatatttgacat, ttgtgtccccgcacta, cttagacaggtctcaa, atcccttatatgttgg, ttattcttatcctagc,
ccagctttttcaagga, gacttccttcctgacg, agaaggactgctcttt, atgtagcaacgagaga, tgaccagaacctaatc,
ctacagtggtgggttc, agtttatgcataaagc, agagagtccttttagc, acgatatattaaccca, ggtcttactgctatag,
actaggtctgatatta, tgccctgtacggggct, gatctcctactaacat, aagatagagtcactta, actagtcactaaaaca,
aatttttcagcgatgt, tgatctaaataggagc, gcatctggcttctggt, gatgcctataacataa, actacagtggtgggtt,
cccttagcctggaggc, gcttcttgccgtgaaa, gcaggatgatatcagt, ttagaaccaccgattt, taatggttacagcgtt,
ccttcaaaccatcatc, tgcccactctaggtat, ttccagggtattagta, ccgctcccggctatag, acactgtaggtgtaag,
ttttgcgaaaagaagg, catgcttaaaccccgt, gggctccttagcactg, aatgcacactctttgg, actgagatgactaact,
ccacttagatttggag, ttagggaggagtaagc, tccatctcagacatac, tcatacctatagcttt, cttccacctgtaccac,
aaagtggcgcagtgat, cgattgaagatgagat, tcttttgccctttcgc, cttaatcctctatttg, tataatctgcgtggag,
aacctctgatttgtgt, aatagattccttacct, cacagaacgttttatc, ctgatctgtagaaggc, cttttagaggctctcg,
agagccaggcagttac, gggcacagtcggtaag, tgtcatctttcgtttc, acttttaggccctcag, ctcaaagatttggttc,
cgtcagatctgatgag, actttagtgtatcatt, gagcttgaagcgtagg, caactaagcagttcaa, gatatatcggaattaa,
tcgtatctgtgatgaa, gttttttgtgcaccca, agggatgaggtattac, ccttcctgacgcagat, ttgttcgggcccaggt,
caggcgttgtgcttaa, agcacgcagtcacctc, tgacaaggtttaaggc, aggcttgtggaacccc, tcatttgcttaccaac,
tgatcacacccttatg, ctaatttagcatccga, ggtatatgcactcaca, tacccttctccttcgt, acgatcaaaaaaaagg,
acctacaacctactca, gaactggcttaggtaa, ctaagcccgccccccc, caattacgtaattatt, tagcaataccttttta,
caaccatatcttgtag, gaaaagttttacagcc, attcatgggaaccctt, actcgggaagactggg, ctggaggcgtagcttg,
atctgcaaatgacctg, cccctctaccttaggt, cgaaatgtgggttttt, tcctaaaacagcggtc, attcgttgttattaca,
ccacccttcacctaca, ttaggttctccctaac, aataaactccaatagc, ttctgacttcgttagc, tgtatccaatccaagt,
cttctctcttatcaac, ttaaacaggctatagc, ggctggctcttaatcc, gttagacatttcaatg, agaccaaaactctaac,
atcagcgattttaaaa, atcaggtttctgtgcc, aacgtacgatcatcct, tcatgcgctgaggagt, ttattaaactctggga,
ttaggaacagtgtcca, accgagcagagaccct, gtcacatcaatacgta, aaggaaagcgattcca, tgtcgatgagcgctag,
agctctatctcattgc, taccgtcaaacttaac, tcatagagatgcagta, taaatacctaagcgat, gggcagcagcttagaa,
caattcgttttacaga, tgcaagtgcttaaagc, tagggttttcccttac, gcccaggttggcgggg, agggcatcccccccca,
gggaattggactagtt, accataagtgacaaca, ggtgatcttttgcttg, acttcagggtttatac, ggttaaggggaaggta,
acgccatggcaggccc, attttggtccggctgg, aactctcgcatccttg, cggctgccccttaccc, tattctagtggtatat,
ggagatttagtagagt, ttggaactgcaaactt, gagcaagttggagagc, tgataacatgtgtggc, aaacttatgtttgata,
aagccggtccataatt, gacaacaatggccgtt, agctcctcctcttagc, tgagctgagtcatttc, aaggagcctttaaatc,
acattttttccgtaat, tccggaccctaattta, tgagagaaattcctca, aacataactgtcagaa, aactccccgttgggcc,
aagcaactcaaagatc, cgttccacagaaaatc, tgttcagattcctgca, catgctgactcaacat, tctctggtactcagga,
gtcatctccggccagg, atgccaataactgtag, aacagaactgtactga, gttgcctccagaccct, ttatacgttctcttag,
aagattactggattga, caaaagcccaatctcc, tgggcgatcactgaag, tgtttgggctaagaag, tcggagttgaagacca,
accgaaggaataatag, cacatctattccctgt, agggtgagatcagata, gggccgctgcgagtgc, cattatatgtaggcgt,
acgtcagcttgcaagg, cttttaggccctcaga, acacaatatggctcga, tattatctctctttgg, agctggtttcctgcgt,
atgatgagcaagaggc, cctcctggattgaaag, aagacactggcaccac, gtgctgttcactctat, ggacaacaggttgaaa,
agttaggcatgttgct, cccccagcaacagtac, tttaggtactcacata, aaatgatttgcgtgaa, gcaataatggattaaa,
tccagggagctcaact, cttacttgttctattg, cttggtagatcttcga, cctataggttcagtgg, ccctaatttagcatcc,
tgtagatgtgcaaacg, ggccgttgaatcccat, gcgttggcgttatctc, tcaccacaatttccat, agggtggatctcctct,
atgcaagaggtgccca, tcagtccttggctacc, caggactcctcaacag, gggtgtacgtgacccc, tctgtggagcaattcc,
gcttggtgtgccagac, ttcacgactctgaaaa, gagccccagccttgag, gctttgctacccaggt, agtcatgaggttcagt,
gagctggtcaccttta, tcctaagcccgccccc, gttgttccaaaggtta, gtctgacggctccgga, acgtctttcatagata,
tgggtaaactcaatca, aaaacttatcttgttt, cgttgaatcccattgc, gaaagttgcattatga, caggcgggcgattcgc,
gcccacgtggttaaac, aagtctccagtagcca, taattgaacctggaca, cacattcgtctcaagt, gttcacgctcattttg,
ttaccatctgattgtc, gtcgatgagcgctaga, agtaaatttttgtagc, tccaccagatttgcag, ccctcgacgctggcgt,
tttgaggacacatctc, cttatgtgtaaattgc, accggtagagacgctc, gcaatgatgtgttcgg, gtctccatcacgacta,
tggcgtctctgtacct, ttgccctgcaagtatg, aggacaggtgatgtag, caacctcgcactccta, ttcctttgaccaaggt,
acttatactccagggg, taggatttgttaaagt, aaccagatgtccatgc, ccatgacacctaacat, acttgcccatcattac,
taagcccgcccccccc, ccaccttgaaaatcgt, ccggaccctaatttag, ccctgtcttcctcgtc, gataaacgcttgaacc,
gatgtgcaaacgtacg, tcccttgcccttagtt, atgtaataatcccaat, tgtctaatctcataca, tctttagcgagtctac,
ttaggcatccagcccc, atcacgacctttaaaa, tgccaataactgtaga, ggtatatgtctaaaac, tttgaagaggatagat,
attgttgtacaagcag, acatgttggccctgta, agacggataagacagc, agaagctcagtgaccg, ttattttgagccctgt,
ttatgggtctttgaac, aacattatatgtaggc, ggcccacgtggttaaa, atcttctctaccgggt, cacaaaagaatctggt,
ggctacttaacatgat, tttaccccaggagatt, tggctacttaacatga, atgccgacacacttta, tctaatttgttaggac,
cattaatgattattgg, tagctttttcatacga, tcacatcgggtcagat, gtgtgtactgatctct, taaagagtgcctacat,
tgcgccaagccccagg, acccttctccttcgtc, cactccagcgttgaca, gttagatgatgtcaca, agctttgtaggtgaat,
gaacagctacttgtgt, actccccgttgggccc, cccagattgcggcagt, ttaaccaaccaaatac, ccctgcatgattgagg,
tttgacgagctgagat, ggcacctcagaacgtt, ttggcgtgtgttacag, gtggtgggattcagac, caccgaaaacaattta,
tagtgggccaagagtg, cagagcccaaacctgt, ataaccagttctgaag, attagctgctgggatc, atgattttggtcctaa,
ctatgtaacaacgaat, gtagaccgtatatttg, agaaccaaccaataaa, cagctccacgtctttc, ctttagtatttctgag,
tagtaagtttgtacaa, atggtaacaaccctta, gcataaacgtcagctt, tttctctaagagcgaa, ccaggcatgtaaggtg,
gtcggtaatccaagct, tgagtttgtaccagac, ctgacagcatgatctg, ttccctaagttgcact, gtgtctccttgttgca,
tttggtctgagctggg, tacacatactaaatag, ctgtggatcaataaca, tccaaccctattagat, ggcgcctggggactct,
atgatagacgaggaat, cttggatgaatcaaca, acaaggagtctaaggt, ctaaatcttagaagta, ggtggatgggagcatc,
attgtgtcactttcta, tcaagctcagtgttgc, ctccttgcgggggggg, tgtctatgagcgctcc, ttcagagaccgaggac,
tcgataaatcttactt, gaaagtggcgcagtga, ctattatacgcacaag, gtgggttagagatatt, gcttggagcttaagaa,
cttatgtagcaacgag, attctattggcactct, actgatgccatctccc, taacaccagtcctggc, cgtcaaacttaacagc,
tggtatcagcagcaaa, acaaagcacagcttgc, gtaattaacaataaga, cagttagttcatctcc, tacccacacccatagg,
ggcgctattctgagcc, aggttaacatactatt, tacttcacgttcttcc, aggcgcctggggactc, ctttaggtactcacat,
ggttggtgagcactgg, taggacatcagatttt, gcaattgtactgagtt, ccaacccggttaaacc, aaacgtacgatcatcc,
actgaaagcgatacca, ttaccaagtcttgtaa, ctttgcttctttccgt, aacatccgccgcccag, tattgctgagaatccc,
ggcaatacaactgtat, agacggctcctagaag, aagatttgaggacctc, tggtaacaacccttaa, tattatattcgaagaa,
gcaattttggatctta, aatgagctccccttta, ggtgagttaaaggggt, acagctccacgtcttt, cggctcataatctgta,
cctcagaacgtttgcc, aatggggggggaaact, cagtacccaacactgc, ttaaaaaagagagcgt, taatccactattgtgt,
gagtgagtcttgctat, ctatgagaatatgcga, ggctatatcatgtgtg, gaccccaactctatca, tctaggactcaacttc,
aaaccatgtctaaaag, atctgaaagggttgtc, agcggcagaatggcat, aacacgttatacacat, aagctctatggctttg,
ggagtgcgttggcgtt, tcggtaatccaagcta, tgtagttcttggaatc, tcagtgctcggaaaag, gaaatatgttggatac,
cactagaccaatagta, taatacgagtccactg, acacttaggatgtatc, ggtctgacggctccgg, cctcccgctttcacgc,
ttctacagctctgtct, ttccctttatgatata, tctcttatgacctttg, ttgaaaatctagttgt, gcaatccccaggttca,
aacataacccaatgaa, ctggttacagagccag, ctggtacttattacat, ttacatgaggcctggc, caaatttcccaggcta,
cgccagctcccaggaa, tagtaaaaattcacgg, cagttcttagtttagg, taacttaacaatttac, gccatcgtgcgaggcc,
taatacatctgagtgc, gactgtacttggatca, tggctcggtcgcctcc, acgtggcaccagatgg, cgttagtgaaatatga,
atactgtaatgtctga, tttacatgaatagtcg, cgaagacctagctcag, tcaaaaggagggcacc, ataggcggattacttg,
tttcccgcaagaacag, gctttcatggtagggt, tagggctcctggacgt, agccatcattccttag, cagtgtgtcatagatg,
gcctaggtgcaaaagg, ctaaggggggcacagc, tgctggcaatgtcgag, cacttgcccatcatta, caacatgtcaacctat,
tttgaagggcaaaacg, gcaaatggtagaagac, ccttgttgtctcaact, ctctatttcgttttat, catatctccttccacc,
tggttgcttaggagac, tcttggtcgtttctgc, gctgatttaaaatggt, aggcgctattctgagc, accagatctggcgaga,
ggatgaggtattacca, gtaggtgtatacccat, atccatccgataaaag, tcccacaatgtaggag, acatgcttaaaccccg,
cataccctggaagaat, agctccctcagaaggt, acattatatgtaggcg, gagtcctggcacaagt, gattacccctgacatc,
tatgaggttcaagttt, ctcagaacgtttgcct, gttgagcacctttgat, attgatgttagagaac, cttaggtctatgtgcc,
tccatccgccttgcct, ccatagtaattctcag, ttgtactcacacgatt, aatagggatgaactac, ctggtgtaggtctgtt,
aagtctgattcttgta, tttgcgcttcctcctt, atataatctgcgtgga, aggaggaaagcgtgtt, tggtctgtgtaggaat,
ttagacacaggcgcct, cctccatctaggcact, gatactatgaggcagc, tgtccattaacataca, gacctagattaccaag,
gaattgactattatag, cagtgacaacgaataa, cgaaagacatgagaac, acctatatccgcctct, tggtgaggctggtagc,
ctttcagggtttgtgc, ccagtgtcctccctac, attttcgtaaaagttg, acacacccttttaagt, tttgtgttttttgccg,
ccttctaaaaaaagcc, gatggttattctttgc, gctgcatgtaatctgc, aagctggggtgtgaaa, caacatgacctggtaa,
tgttaggcgaactatt, tgtgctgcatgtaatc, agcctgtctgagagga, ttccttatgattaatc, ccttatcaactgaatt,
ggcttatgtaaacatc, aagtaaacctttccta, ctgaggggtggttgag, cgtctgaaaaaaagag, acaatgctttaatagg,
ctttttttttcgttgc, tattctgtgtacgcta, tccacatgaaaagacc, gtctatatagtgtaat, cttttcataatctcac,
caccacaataggcccc, tttccccctcctccga, ccctgccagattgtca, tccaggcattaaagtg, cctacaacctactcat,
gtagatacgagtttta, tatgccaataactgta, aactgagaaatatgac, aattaccgtcaaactt, ttatatcttatgactg,
ttaagtttttaatcgt, ggtcctcctccctgta, atgggattggtgggac, atcgaaattatattca, taacactaggttatta,
tcttaggacttgactt, tgggattccgtctgtg, tctcttacaactgttt, tttagcgagtctacca, tctttccctgagtcac,
tccaatacaattgacc, gaacgaggggtaaaaa, tgtttaagttagcaag, aaccccttatgatggg, gaacctatatccgcct,
tgcgttggcgttatct, gaccctcgacgctggc, gtgactcatcctttag, gcatctaagtatgaac, gaaccagttgtgacta,
gcctgaactcgatttt, aattgcttttattggc, ctttaagcggttttgt, ggagttagccaccgtt, tacgtctttcatagat,
acattgttgggtcact, tatatccgcctctctt, acttcttgtatccgct, gagctatgtcccctta, gcgatcactgaaggtc,
tttgaactctcacgtc, agatgttcattagtgg, ttaacttctgcagtaa, ccgccttgcggtctcc, gagagagatctgaggc,
tctcttcagtctcgtg, accctcgacgctggcg, aagggttactgacatt, tacccgccagctccca, ggtgcttagccatggg,
tttaaagccctaaggt, aagagttaggtaacaa, tgaggcgggcttatgc, tctctttattcgtgca, atgccattctcctttg,
tcatctgcaacctaac, cagtactaggtcctgg, tggccaagaaggttaa, ccaacaacatagctcc, gaagacctggcctgat,
acgggtgcatgtaatc, ttgcatcttagacaat, ttaccagaggtcttgc, cacaatgtaggagggg, gtagaggaaagtggcg,
atttctttgcacgcag, gtttcaggtaattctg, tgtactaggaaggaca, ctcgcatccttgaatt, cctcgacgctggcgtt,
ataatctgcgtggagc, tttatgcataaacgtc, gtttggttaggtaaac, gtcaccttcaactttc, gcctgtattggagtcc,
tcttggtagatcttcg, tagtggtaatgttatg, gttccctccattatgt, tttgtagaaggatggt, actaaggatagtttcg,
ttatcattccacacag, acaagtaaggaaggta, tatttaaacgcaaatt, ttacggggtacattag, attgaatagatttcct,
taaacgtcagcttgca, gcacgtggcaccagat, gtctcctttcgtgata, atcattgcccatagac, tgtttccgttataaat,
gccattttatggagaa, gttaacgtatttttca, gttaactactgtttag, gacggagtcactatga, acatgagaggatagag,
gatgggtaggaggtac, tgtgcagataaaaccc, aatcgatttgagaaat, tcatctgtccctgggt, atccattatcttgggt,
aacgagatataatctg, ttgggcggcttaggca, aatctaaacccctccc, aaaaacagcatgctga, atgtcttttttacaag,
cttaacatgatgaagc, gcttagtctctgaaag, agtatgccaataactg, aaccacgtgttacctg, atctataaaaggacgt,
tccagcataaaaaatg, cctttagtgcctctgc, ccaccgccatacttca, atcaatggagggggtg, ccagtcctttaaagct,
cggtaaggttcagatt, cctaaccttatgtagc, tctcccttatttgaca

### REFERENCES

1. Ackerman et al. 2020. “Massively Multiplexed Nucleic Acid Detection with Cas13.” Nature 582 (7811): 277-82.
2. Alileche et al. 2012. “Nullomer Derived Anticancer Peptides (NulloPs): Differential Lethal Effects on Normal and Cancer Cells in Vitro.” Peptides 38 (2): 302-11.
3. Alileche et al. 2017. “The Effect of Nullomer-Derived Peptides 9R, 9S1R and 124R on the NCI-60 Panel and Normal Cell Lines.” BMC Cancer 17 (1): 533.
4. Augustus et al. 2020. “The Art of Obtaining a High Yield of Cell-Free DNA from Urine.” PloS One 15 (4): e0231058.
5. Barbany et al. 2019. “Cell-Free Tumour DNA Testing for Early Detection of Cancer—a Potential Future Tool.” Journal of Internal Medicine 286 (2): 118-36.
6. Barry, Michael J. 2001. “Prostate-Specific-Antigen Testing for Early Diagnosis of Prostate Cancer.” New England Journal of Medicine. https://doi.org/10.1056/nejm200105033441806.
7. Battaglin et al. 2018. “Microsatellite Instability in Colorectal Cancer: Overview of Its Clinical Significance and Novel Perspectives.” Clinical Advances in Hematology & Oncology: H&O 16 (11): 735-45.
8. Bell et al. 2015. “Cancer. The Transcription Factor GABP Selectively Binds and Activates the Mutant TERT Promoter in Cancer.” Science 348 (6238): 1036-39.
9. Bowler et al. 2018. “Hypoxia Leads to Significant Changes in Alternative Splicing and Elevated Expression of CLK Splice Factor Kinases in PC3 Prostate Cancer Cells.” BMC Cancer 18 (1): 355.
10. Bronkhorst et al. 2019. “The Emerging Role of Cell-Free DNA as a Molecular Marker for Cancer Management.” Biomolecular Detection and Quantification 17 (March): 100087.
11. Cackowski et al. 2018. “Minimal Residual Disease in Prostate Cancer.” Advances in Experimental Medicine and Biology. https://doi.org/10.1007/978-3-319-97746-1 3
12. “Cancer.” n.d. Accessed Feb. 6, 2021a. https://www.who.int/news-room/fact-sheets/detail/cancer. n.d. Accessed Dec. 2, 2020b. https://www.who.int/cancer/detection/en/.
13. Chen et al. 2021. “Cell-Free DNA Concentration and Fragment Size as a Biomarker for Prostate Cancer.” Scientific Reports 11 (1): 5040.
14. Consortium et al. 2012. “An Integrated Encyclopedia of DNA Elements in the Human Genome.” Nature 489 (7414): 57-74.
15. Ding et al. 2019. “Saliva-Derived cfDNA Is Applicable for EGFR Mutation Detection but Not for Quantitation Analysis in Non-Small Cell Lung Cancer.” Thoracic Cancer 10 (10): 1973-83.
16. El-Haibi et al. 2013. “Differential G Protein Subunit Expression by Prostate Cancer Cells and Their Interaction with CXCR5.” Molecular Cancer 12 (June): 64.
17. Etzioni et al. 2003. “The Case for Early Detection.” Nature Reviews. Cancer 3 (4): 243-52.
18. Georgakopoulos-Soares et al. 2020. “Absent from DNA and Protein: Genomic Characterization of Nullomers and Nullpeptides across Functional Categories and Evolution.” Cold Spring Harbor Laboratory. https://doi.org/10.1101/2020.03.02.972422.
19. Hampikian et al. 2006. “ABSENT SEQUENCES: NULLOMERS AND PRIMES.” In Biocomputing 2007, 355-66. WORLD SCIENTIFIC.
20 Hawkes, Nigel. 2019. “Cancer Survival Data Emphasise Importance of Early Diagnosis.” BMJ 364 (January). https://doi.org/10.1136/bmj.1408.
21. Heidenreich et al. 2014. “TERT Promoter Mutations in Cancer Development.” Current Opinion in Genetics & Development 24 (February): 30-37.
22. Heitzer et al. 2020. “Cell-Free DNA and Apoptosis: How Dead Cells Inform About the Living.” Trends in Molecular Medicine 26 (5): 519-28.
23. ICGC/TCGA Pan-Cancer Analysis of Whole Genomes Consortium. 2020. “Pan-Cancer Analysis of Whole Genomes.” Nature 578 (7793): 82-93.
24. Inoue et al. 2015. “Decoding Enhancers Using Massively Parallel Reporter Assays.” Genomics 106 (3): 159-64.
25. Jiao et al. PCAWG Tumor Subtypes and Clinical Translation Working Group, Alexandra Danyi, et al. 2020. “A Deep Learning System Accurately Classifies Primary and Metastatic Cancers Using Passenger Mutation Patterns.” Nature Communications 11 (1): 728.
26. Ji et al. 2014. “Methylated DNA Is over-Represented in Whole-Genome Bisulfite Sequencing Data.” Frontiers in Genetics 5 (October): 341.
27. Karczewski et al. 2020. “The Mutational Constraint Spectrum Quantified from Variation in 141,456 Humans.” Nature 581 (7809): 434-43.
28. Kellner et al. 2019. “SHERLOCK: Nucleic Acid Detection with CRISPR Nucleases.” Nature Protocols 14 (10): 2986-3012.
29 Koulouras et al. 2021. “Significant Non-Existence of Sequences in Genomes and Proteomes.” Nucleic Acids Research, March. https://doi.org/10.1093/nar/gkab 139.
30. Lee et al. 2020. “BRCA1/BRCA2 Pathogenic Variant Breast Cancer: Treatment and Prevention Strategies.” Annals of Laboratory Medicine 40 (2): 114-21.
31. Lennon et al. 2020. “Feasibility of Blood Testing Combined with PET-CT to Screen for Cancer and Guide Intervention.” Science 369 (6499). https://doi.org/10.1126/science.abb9601.
32 Muñoz-Maldonado et al. 2019. “A Comparative Analysis of Individual RAS Mutations in Cancer Biology.” Frontiers in Oncology 9 (October): 1088.
33. Murray, Nigel P. 2018. “Minimal Residual Disease in Prostate Cancer Patients after Primary Treatment: Theoretical Considerations, Evidence and Possible Use in Clinical Management.” Biological Research 51 (1): 32.
34 Nik-Zainal et al. 2016. “Landscape of Somatic Mutations in 560 Breast Cancer Whole-Genome Sequences.” Nature 534 (7605): 47-54.
35. Ohkia et al. 2004. “Evidence for Prostate Cancer-Associated Diagnostic Marker-1: Immunohistochemistry and in Situ Hybridization Studies.” Clinical Cancer Research: An Official Journal of the American Association for Cancer Research 10 (7): 2452-58.
36. Poulos et al. 2015. “The Search for Cis-Regulatory Driver Mutations in Cancer Genomes.” Oncotarget 6 (32): 32509-25.
37 Powter et al. 2021. “Human TERT Promoter Mutations as a Prognostic Biomarker in Glioma.” Journal of Cancer Research and Clinical Oncology 147 (4): 1007-17.
38. Prior et al. 2012. “A Comprehensive Survey of Ras Mutations in Cancer.” Cancer Research 72 (10): 2457-67.
39. Qin et al. 2014. “The Tumor Susceptibility Gene TMEM127 Is Mutated in Renal Cell Carcinomas and Modulates Endolysosomal Function.” Human Molecular Genetics 23 (9): 2428-39.
40. Razavi et al. 2019. “High-Intensity Sequencing Reveals the Sources of Plasma Circulating Cell-Free DNA Variants.” Nature Medicine 25 (12): 1928-37.
41. Sadeh et al. 2021. “ChIP-Seq of Plasma Cell-Free Nucleosomes Identifies Gene Expression Programs of the Cells of Origin.” Nature Biotechnology, January. https://doi.org/10.1038/s41587-020-00775-6.
42. Saghafinia et al. 2018. “Pan-Cancer Landscape of Aberrant DNA Methylation across Human Tumors.” Cell Reports 25 (4): 1066-80.e8.
43. Santoni et al. 2020. “In the Search of Potential Epitopes for Wuhan Seafood Market Pneumonia Virus Using High Order Nullomers.” Journal of Immunological Methods 481-482 (June): 112787.
44. Song et al. 2019. “Small-Molecule-Targeting Hairpin Loop of hTERT Promoter G-Quadruplex Induces Cancer Cell Death.” Cell Chemical Biology 26 (8): 1110-21.e4.
45. “The Cancer Genome Atlas Program.” 2018. 2018. https://www.cancer.gov/tcga.
46. Tung et al. 2018. “BRCA1/2 Testing: Therapeutic Implications for Breast Cancer Management.” British Journal of Cancer 119 (2): 141-52.
47. Ulz et al. 2019. “Inference of Transcription Factor Binding from Cell-Free DNA Enables Tumor Subtype Prediction and Early Detection.” Nature Communications 10 (1): 4666.
48. Valencia et al. 2011. “Role and Expression of FRS2 and FRS3 in Prostate Cancer.” BMC Cancer 11 (November): 484.
49. Vergni et al. 2020. “The Farther the Better: Investigating How Distance from Human Self Affects the Propensity of a Peptide to Be Presented on Cell Surface by MHC Class I Molecules, the Case of Trypanosoma Cruzi.” PloS One 15 (12): e0243285.
50 Vergni et al. 2016. “Nullomers and High Order Nullomers in Genomic Sequences.” PloS One 11 (12): e0164540.
51. Vinagre et al. 2013. “Frequency of TERT Promoter Mutations in Human Cancers.” Nature Communications 4: 2185.
52. Vita et al. 2019. “The Immune Epitope Database (IEDB): 2018 Update.” Nucleic Acids Research 47 (D1): D339-43.
53. Warton et al. 2015. “Methylation of Cell-Free Circulating DNA in the Diagnosis of Cancer.” Frontiers in Molecular Biosciences 2 (April): 13.
54. Worm et al. 2018. “Review of Blood-Based Colorectal Cancer Screening: How Far Are Circulating Cell-Free DNA Methylation Markers From Clinical Implementation?” Clinical Colorectal Cancer 17 (2): e415-33.
55. Zill et al. 2018. “The Landscape of Actionable Genomic Alterations in Cell-Free Circulating Tumor DNA from 21,807 Advanced Cancer Patients.” Clinical Cancer Research: An Official Journal of the American Association for Cancer Research.

LENGTHY TABLES
The patent application contains a lengthy table section. A copy of the table is available in electronic form from the USPTO web site (https://seqdata.uspto.gov/?pageRequest=docDetail&DocID=US20240229157A1). An electronic copy of the table will also be available from the USPTO upon request and payment of the fee set forth in 37 CFR 1.19(b)(3).

1. A method of identifying one or a plurality of nullomers in a sample comprising:
(a) isolating a plurality of nucleic acids from the sample;
(b) contacting the nucleic acids to one or a plurality of probes specific for one or a plurality of nullomers;
(c) detecting the presence of the probes associated with the one or plurality of nullomers; and
(d) correlating the presence or quantity of probes with the likelihood of the presence or quantity of nullomers in the sample.

2. The method of claim 1 further comprises, prior to step (b), disassociating a plurality of double stranded nucleic acid sequences comprising at least one nullomer by exposing the double-stranded nucleic acid sequences to a predetermined melting temperature for a period of time sufficient to create single stranded nullomer, annealing at least one primer to the nullomer, and allowing a sufficient period of time to extend the primer in the presence of dNTPs and DNA polymerase.

3. The method of claim 2, wherein the steps of disassociating a plurality of double stranded nucleic acid sequences comprising at least one nullomer by exposing the double-stranded nucleic acid sequences to a predetermined melting temperature for a period of time sufficient to create single stranded nullomer, annealing at least one primer to the nullomer, and allowing a sufficient period of time to extend the primer in the presence of dNTPs and polymerase are repeated multiple times such that copies of the at least one nullomer are produced.

4. The method of claim 1, wherein the probe or plurality of probes comprise a complementary nucleic acid sequence bound to or associated with a fluorescent molecule, radioactive isotope or chemiluminescent molecule.

5. The method of claim 1, wherein the step of detecting is performed by mass spectrometry.

6.-30. (canceled)

31. A method of preparing a sample from a subject free of clinically presented cancer symptoms comprising:
a) isolating nucleic acids from the sample; and
b) analyzing the nucleic acids with a probe specific for at least one nullomer chosen from Table 1.

32.-33. (canceled)

34. The method of claim 31, wherein:
i) step (b) further comprises calculating one or more scores based upon the presence, absence, or quantity of the at least one nullomer; and
ii) step (b) further comprises correlating the one or more scores to the presence, absence, or quantity of the at least one nullomer such that, if the amount of the at least one nullomer is greater than the quantity of the at least one nullomer in a control sample; or, if the amount of the at least one nullomer is substantially equal to the quantity of the at least one nullomer in a sample taken from a subject known to have a hyperproliferative disorder, then the subject is diagnosed as having a hyperproliferative disorder.

35. The method of claim 31, wherein step (b) comprises detecting at least one nullomer by DNA sequencing, quantitative real-time reverse transcription-PCR (qRT-PCR), isothermal amplification, microarray, multiplex nullomer profiling assay, RNA-ish, or northern blotting.

36.-41. (canceled)

42. The method of claim 31, wherein the sample is free of cells.

43. A method of diagnosing a subject with cancer comprising:
(a) contacting a plurality of nucleic acids from a sample to a system comprising a probe specific for one or a plurality of nullomers; and
(b) detecting the presence of or quantifying the amount of one or more nucleic acids from the sample.

44. The method of claim 43, wherein the method comprises detecting the presence, absence or quantity of one or a plurality of the nullomers provided in Table 1.

45. The method of claim 43, wherein the method comprises detecting the presence, absence or quantity of nullomers that comprise at least 93% sequence identify to one or a plurality of the nullomers provided in Table 1.

46. The method of claim 43, wherein the at least one nullomer is detected by qRT-PCR or CRISPR diagnosis.

47.-48. (canceled)

49. The method of claim 43 further comprising, after the step of detecting:
(i) normalizing the quantity of the probe as compared to a quantity of signal from a negative control; and
(ii) correlating the one or more scores to the presence, absence, or quantity of the at least one nullomer such that, if the amount of the at least one nullomer is greater than the quantity of the at least one nullomer in a control sample; or, if the amount of the at least one nullomer is substantially equal to the quantity of the at least one nullomer in a sample taken from a subject known to have a hyperproliferative disorder, then the subject is diagnosed as having a hyperproliferative disorder.

50.-51. (canceled)

52. A kit comprising one or more probes or primers for detecting the presence, absence or quantity of one or a plurality of the nullomers provided in Table 1 or nullomers that comprise at least 93% sequence identify to one or a plurality of the nullomers provided in Table 1.

53. The kit of claim 52, wherein the one or more probes comprise one or a combination of the nullomer sequences of Table 1 or complementary thereof.

54. A computer program product encoded on a computer-readable storage medium, wherein the computer program product comprises instructions for:
a) detecting the presence, absence or quantity of at least one nullomer in a sample of a subject;
b) normalizing the presence, absence, or quantity of the at least one nullomer in the sample against the presence, absence or quantity of the at least one nullomer in a control sample; and
c) correlating the presence, absence, or quantity of the at least one nullomer in the sample to a likelihood that the subject having a hyperproliferative disorder.

55. The computer program product of claim 54 further comprising instructions for calculating a score associated with the presence, absence or quantity of the at least one nullomer in the sample and correlating the score to a likelihood that the subject has a hyperproliferative disorder.

56. The computer program product of claim 54 further comprising instructions for:
a) detecting and normalizing the presence, absence or quantity of a second nullomer in the sample;
b) calculating a combined score associated with the presence, absence or quantity of the at least one nullomer and the second nullomer in the sample; and
c) correlating the combined score to a likelihood that the subject having a hyperproliferative disorder.

57. The computer program product of claim 54, wherein at least 2 different nullomers in the sample are detected, normalized and correlated.

58. The computer program product of claim 54, wherein the presence, absence, or quantity of the at least one nullomer is detected by qRT-PCR amplification.

59. The computer program product of claim 54, wherein the control sample is obtained from a subject free of a hyperproliferative disorder.

60.-63. (canceled)
